(19) United States
(12) Patent Application Publication
(10) Pub. No.: US 2024/0083861 A1
(43) Pub. Date: Mar. 14, 2024

(54) HSD17B13 INHIBITORS AND USES THEREOF

(71) Applicant: FL2022-001, Inc., San Francisco, CA (US)

(72) Inventors: Andrew R. Hudson, San Diego, CA (US); Steven P. Govek, San Diego, CA (US); Johnny Y. Nagasawa, San Diego, CA (US); Iriny Botrous, San Diego, CA (US); Nicholas D. Smith, San Diego, CA (US); Karensa L. Fasanya, San Diego, CA (US)

(21) Appl. No.: 18/247,139

(22) PCT Filed: Sep. 29, 2021
(86) PCT No.: PCT/US2021/052679

§ 371 Date: Mar. 29, 2023

Related U.S. Application Data
(60) Provisional application No. 63/085,849, filed on Sep. 30, 2020.

(51) Int. Cl. C07D 263/57; C07D 413/06; C07D 493/08; C07D 471/04; C07D 519/00; C07D 487/04

(52) U.S. Cl. CPC C07D 263/57; C07D 413/06; C07D 493/08; C07D 471/04; C07D 519/00; C07D 487/04

(57) ABSTRACT

Described herein are compounds that are HSD17B13 inhibitors, methods of making such compounds, pharmaceutical compositions and medicaments comprising such compounds, and methods of using such compounds in the treatment of conditions, diseases, or disorders associated with HSD17B13 activity.

### CROSS-REFERENCE

This application claims benefit of U.S. Provisional Patent Application No. 63/085,849, filed on Sep. 30, 2020 which is incorporated herein by reference in its entirety.

### FIELD OF THE INVENTION

Described herein are compounds that are hydroxysteroid 17β-dehydrogenase 13 (HSD17B13) inhibitors, methods of making such compounds, pharmaceutical compositions and medicaments comprising such compounds, and methods of using such compounds in the treatment of conditions, diseases, or disorders associated with HSD17B13 activity.

### BACKGROUND OF THE INVENTION

Hydroxysteroid dehydrogenase 17β13 (HSD17b13) is a member of the short-chain dehydrogenase/reductase enzymes highly expressed in the liver on lipid droplets. It has been shown to oxidize retinol, steroids such as estradiol, and bio-active lipids like leukotriene B4. Loss of HSD17b13 expression and enzymatic activity is associated with decreased incidence of liver disease. Inhibition of HSD17b13 enzymatic activity can be used for the treatment of liver diseases that result in hepatic inflammation, fibrosis, cirrhosis, and development of hepatocellular carcinoma.

### SUMMARY OF THE INVENTION

In one aspect, described herein is a compound of Formula (I″), or a pharmaceutically acceptable salt or solvate thereof:

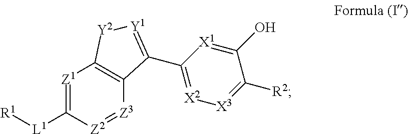

wherein:
X¹, X², and X³ are each independently CR³ or N;
Y¹ is CR⁴ or N;
Y² is N(R⁹), O, or C(R⁴)₂;
Z¹, Z², and Z³ are each independently CR⁵ or N;
L¹ is selected from a bond, —O—, —N(R¹⁰)—, —C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₁₀cycloalkyl and C₂₋₉heterocycloalkyl, wherein C₃₋₁₀cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R³, each R⁴, and each R⁵ are each independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R⁶ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁷ are each independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹⁴, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
R⁹ is selected from H, C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
each R¹⁴ is independently selected C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, C₁₋₆alkyl, C₁₋₆ haloalkyl, C₁₋₆alkoxy, C₃₋₆cycloalkyl, C₂₋₉heterocycloalkyl, and C₁₋₉heteroaryl.

In another aspect, described herein is a compound of Formula (II″), or a pharmaceutically acceptable salt or solvate thereof:

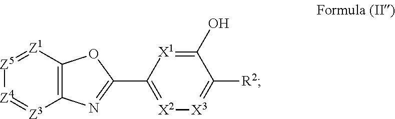

wherein:
X¹, X², and X³ are each independently CR³ or N;
Z¹ and Z³ are each independently CR⁵ or N;
Z⁴ and Z⁵ are each independently CR⁵, CR⁸, or N, wherein one of Z⁴ and Z⁵ is CR⁸;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₁₀cycloalkyl and C₂₋₉heterocycloalkyl, wherein C₃₋₁₀cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R³ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁵ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁷ are each independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
R⁸ is -L¹-R¹;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In another aspect, described herein is a compound of Formula (I′) or Formula (II′), or a pharmaceutically acceptable salt or solvate thereof:

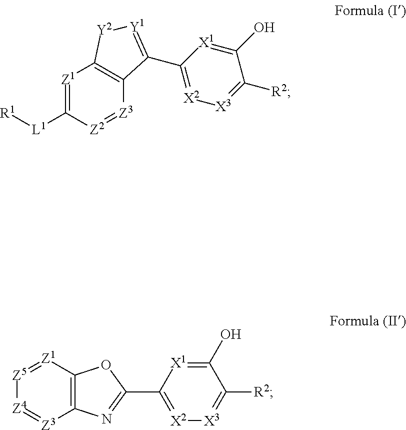

wherein:
X¹, X², and X³ are each independently CR³ or N;
Y¹ is CR⁴ or N;
Y² is N(R⁹), O, or C(R⁴)₂;
Z¹, Z², and Z³ are each independently CR⁵ or N;
Z⁴ and Z⁵ are each independently CR⁵, CR⁸, or N, wherein one of Z⁴ and Z⁵ is CR⁸;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R³, each R⁴, and each R⁵ are each independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R⁶ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁷ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹⁴, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁸ is -L¹-R¹;
R⁹ is selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆ alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
each R¹⁴ is independently selected C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, C₁₋₆alkyl, C₁₋₆ haloalkyl, C₁₋₆alkoxy, C₃₋₆cycloalkyl, C₂₋₉heterocycloalkyl, and C₁₋₉heteroaryl.

In some embodiments is a compound of Formula (I′), or a pharmaceutically acceptable salt or solvate thereof:

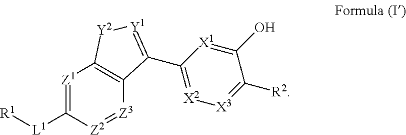

In some embodiments is a compound of Formula (I″) or (I′), or a pharmaceutically acceptable salt or solvate thereof, wherein Y¹ is N. In some embodiments is a compound of Formula (I′) or (I), or a pharmaceutically acceptable salt or solvate thereof, wherein Y² is N(R⁹). In some embodiments is a compound of Formula (I″) or (I′), or a pharmaceutically acceptable salt or solvate thereof, wherein R⁹ is selected from H and C₁₋₆alkyl. In some embodiments is a compound of Formula (I″) or (I′), or a pharmaceutically acceptable salt or solvate thereof, wherein R⁹ is H. In some embodiments is a compound of Formula (I″) or (I′), or a pharmaceutically acceptable salt or solvate thereof, wherein R⁹ is C₁₋₆alkyl. In some embodiments is a compound of Formula (I″) or (I′), or a pharmaceutically acceptable salt or solvate thereof, wherein X¹, X², and X³ are CR³.

In some embodiments is a compound of Formula (I″) or (I′), or a pharmaceutically acceptable salt or solvate thereof, having the structure of Formula (Ia′):

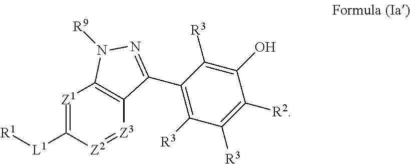

In some embodiments is a compound of Formula (I″), (I′), or (Ia′), or a pharmaceutically acceptable salt or solvate thereof, wherein Z² is CR⁵. In some embodiments is a compound of Formula (I″), (I′), or (Ia′), or a pharmaceutically acceptable salt or solvate thereof, wherein Z² is N.

In some embodiments is a compound of Formula (II′), or a pharmaceutically acceptable salt or solvate thereof:

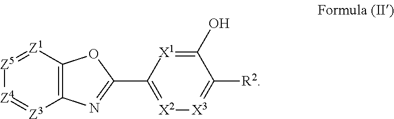

In some embodiments is a compound of Formula (II′) or (II), or a pharmaceutically acceptable salt or solvate thereof, wherein X¹, X², and X³ are CR³. In some embodiments is a compound of Formula (II′) or (II), or a pharmaceutically acceptable salt or solvate thereof, wherein Z⁵ is CR⁸; and Z⁴ is CR⁵ or N.

In some embodiments is a compound of Formula (II″) or (II′), or a pharmaceutically acceptable salt or solvate thereof, having the structure of Formula (IIa′):

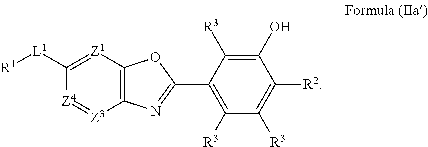

In some embodiments is a compound of Formula (II″), (II′), or (IIa′), or a pharmaceutically acceptable salt or solvate thereof, wherein Z⁴ is CR⁵. In some embodiments is a compound of Formula (II″), (II′), or (IIa′), or a pharmaceutically acceptable salt or solvate thereof, wherein Z⁴ is N. In some embodiments is a compound of Formula (II″), (II′), or (IIa′), or a pharmaceutically acceptable salt or solvate thereof, wherein Z⁴ is CR⁸; and Z⁵ is CR⁵ or N.

In some embodiments is a compound of Formula (II″) or (II′), or a pharmaceutically acceptable salt or solvate thereof, having the structure of Formula (IIb′):

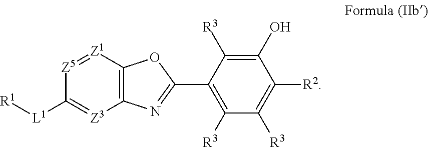

In some embodiments is a compound of Formula (II″), (II′), or (IIb′), or a pharmaceutically acceptable salt or solvate thereof, wherein Z⁵ is CR⁵. In some embodiments is a compound of Formula (II″), (II′), or (IIb′), or a pharmaceutically acceptable salt or solvate thereof, wherein Z⁵ is N. In some embodiments is a compound of Formula (I″), (I′), (Ia′), (II″), (II′), (IIa′), or (IIb′), or a pharmaceutically acceptable salt or solvate thereof, wherein L¹ is a bond. In some embodiments is a compound of Formula (I″), (I′), (Ia′), (II″), (II′), (IIa′), or (IIb′), or a pharmaceutically acceptable salt or solvate thereof, wherein L¹ is —N(R¹⁰)C(O)—. In some embodiments is a compound of Formula (I″), (I′), (Ia′), (II″), (II′), (IIa′), or (IIb′), or a pharmaceutically acceptable salt or solvate thereof, wherein L¹ is —C(O)N(R¹⁰)—. In some embodiments is a compound of Formula (I″), (I′), (Ia′), (II″), (II′), (IIa′), or (IIb′), or a pharmaceutically acceptable salt or solvate thereof, wherein L¹ is —N(R¹⁰)—. In some embodiments is a compound of Formula (I″), (I′), (Ia′), (II″), (II′), (IIa′), or (IIb′), or a pharmaceutically acceptable salt or solvate thereof, wherein Z¹ and Z³ are CR⁵. In some embodiments is a compound of Formula (I″), (I′), (Ia′), (II″), (II′), (IIa′), or (IIb′), or a pharmaceutically acceptable salt or solvate thereof, wherein Z¹ is N; and Z³ are CR⁵. In some embodiments is a compound of Formula (I″), (I′), (Ia′), (II″), (II′), (IIa′), or (IIb′), or a pharmaceutically acceptable salt or solvate thereof, wherein Z³ is N; and Z¹ is CR⁵. In some embodiments is a compound of Formula (I″), (I′), (Ia′), (II″), (II′), (IIa′), or (IIb′), or a pharmaceutically acceptable salt or solvate thereof, wherein R¹ is C₂₋₉heterocycloalkyl optionally substituted with one, two, or three R⁶. In some embodiments is a compound of Formula (I″), (I′), (Ia′), (II″), (II′), (IIa′), or (IIb′), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl, wherein piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl are optionally substituted with one, two, or three R⁶. In some embodiments is a compound of Formula (I″), (I′), (Ia′), (II″), (II′), (IIa′), or (IIb′), or a pharmaceutically acceptable salt or solvate thereof, wherein R¹ is

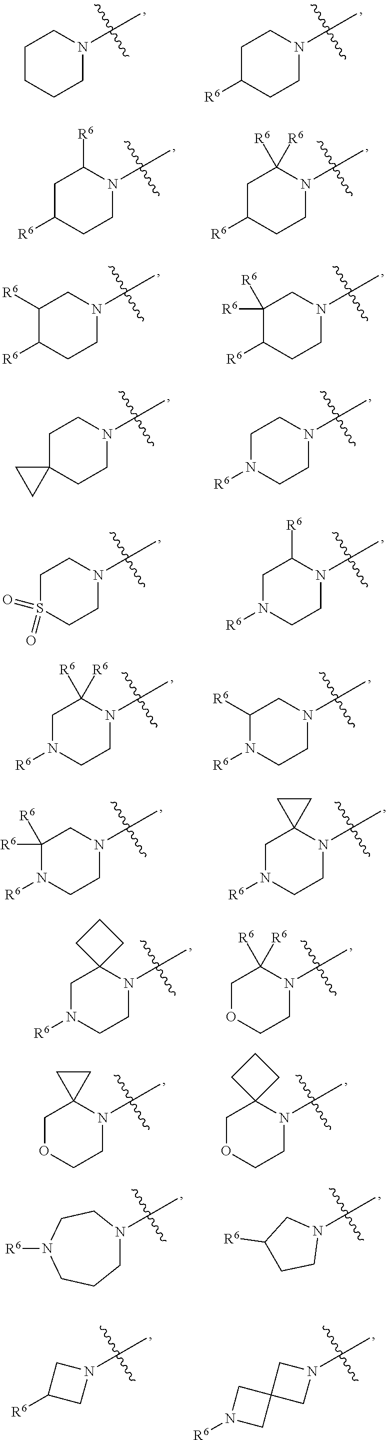

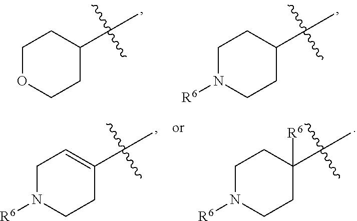

In some embodiments is a compound of Formula (I″), (I′), (Ia′), (II″), (II′), (IIa′), or (IIb′), or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—.

In some embodiments is a compound of Formula (I″), (I′), (Ia′), (II″), (II′), (IIa′), or (IIb′), or a pharmaceutically acceptable salt or solvate thereof, wherein R¹ is

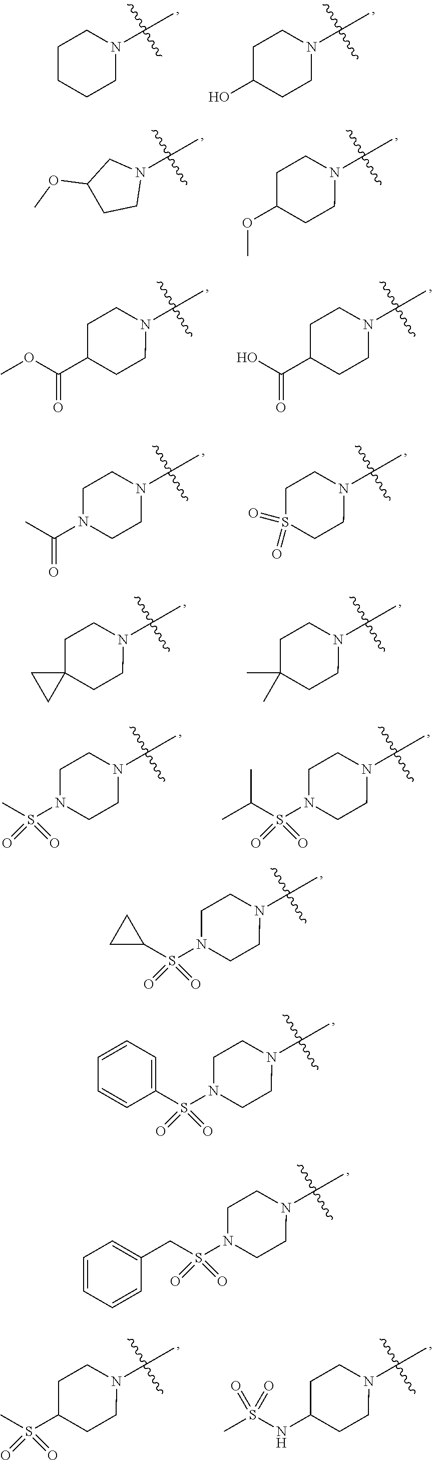

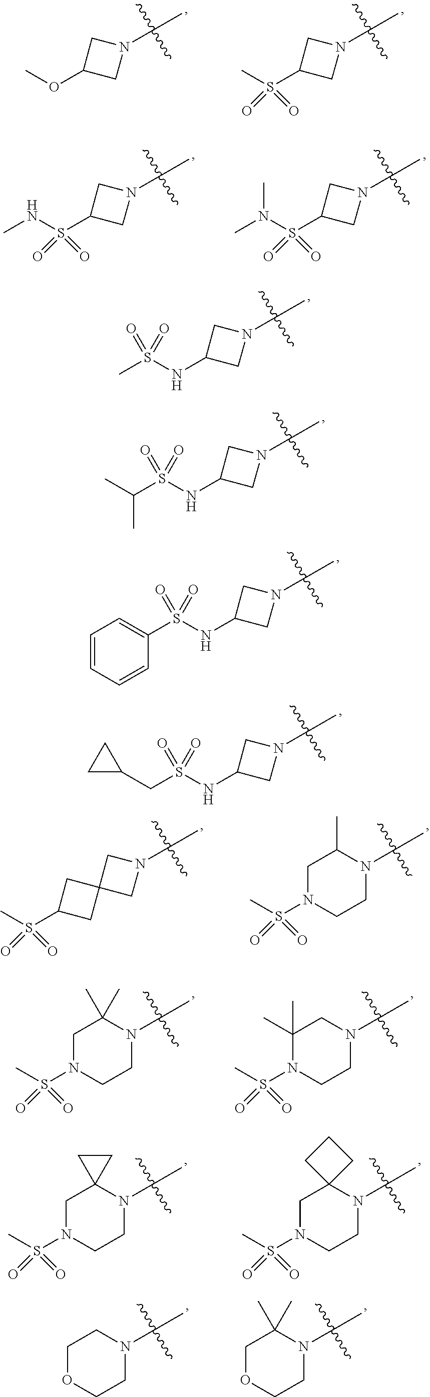

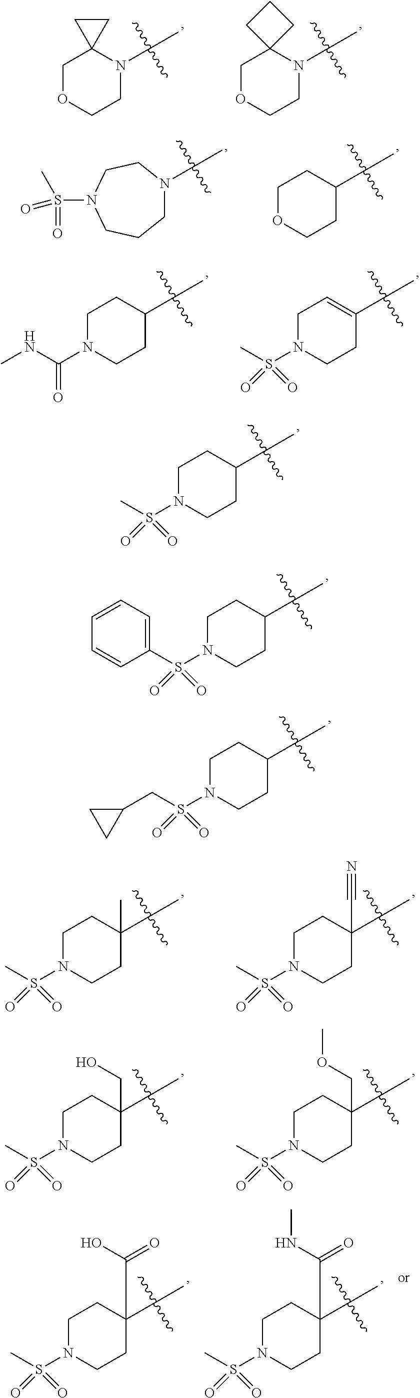

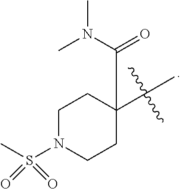 [SMILES: *C1(C(=O)N(C)C)CCN(S(C)(=O)=O)CC1]

In some embodiments is a compound of Formula (I″), (I′), (Ia′), (II″), (II′), (IIa′), or (IIb′), or a pharmaceutically acceptable salt or solvate thereof, wherein R¹ is

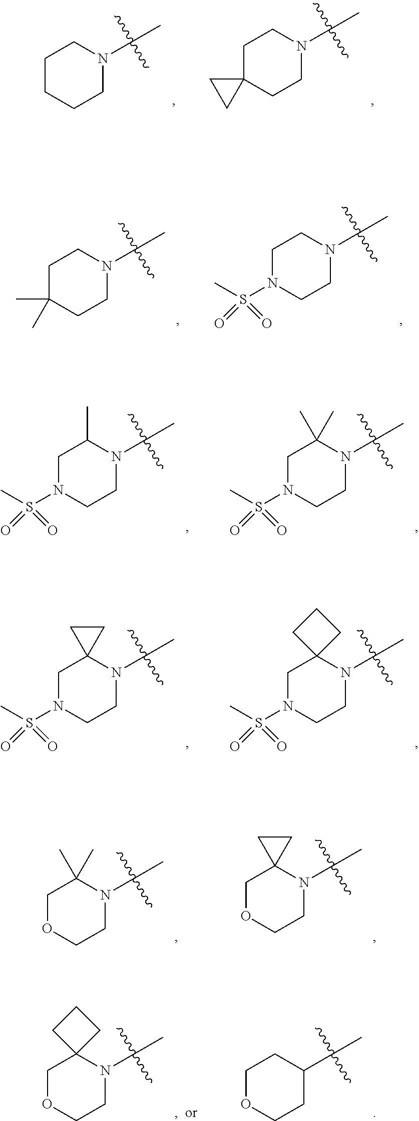

In some embodiments is a compound of Formula (I″), (I′), (Ia′), (II″), (II′), (IIa′), or (IIb′), or a pharmaceutically acceptable salt or solvate thereof, wherein R¹ is C₃₋₈cycloalkyl optionally substituted with one, two, or three R⁶. In some embodiments is a compound of Formula (I″), (I′), (Ia′), (II″), (II′), (IIa′), or (IIb′), or a pharmaceutically acceptable salt or solvate thereof, wherein R¹ is

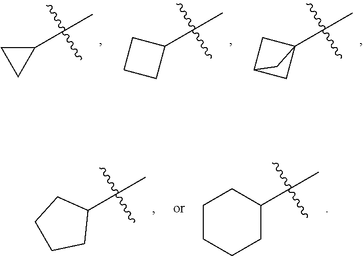

In some embodiments is a compound of Formula (I″), (I′), (Ia′), (II″), (II′), (IIa′), or (IIb′), or a pharmaceutically acceptable salt or solvate thereof, wherein R¹ is C₁₋₉heteroaryl substituted with one, two, or three R⁷. In some embodiments is a compound of Formula (I″), (I′), (Ia′), (II″), (II′), (IIa′), or (IIb′), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷. In some embodiments is a compound of Formula (I″), (I′), (Ia′), (II″), (II′), (IIa′), or (IIb′), or a pharmaceutically acceptable salt or solvate thereof, wherein R¹ is

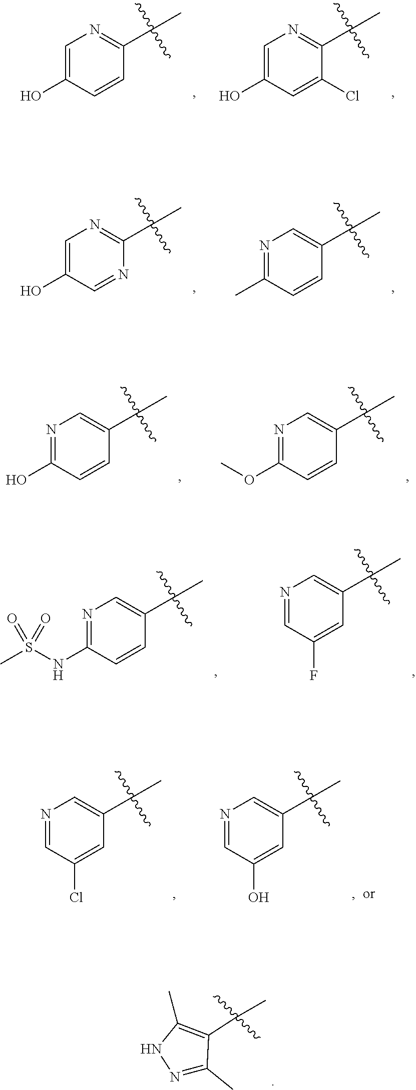

In some embodiments is a compound of Formula (I″), (I′), (Ia′), (II″), (II′), (IIa′), or (IIb′), or a pharmaceutically acceptable salt or solvate thereof, wherein each R⁵ is independently selected from H, halogen, C₁₋₆alkyl, and —OR¹⁰. In some embodiments is a compound of Formula (I″), (I′), (Ia′), (II″), (II′), (IIa′), or (IIb′), or a pharmaceutically acceptable salt or solvate thereof, wherein each R⁵ is H. In some embodiments is a compound of Formula (I″), (I′), (Ia′), (II″), (II′), (IIa′), or (IIb′), or a pharmaceutically acceptable salt or solvate thereof, wherein each R³ is independently selected from H, halogen, C₁₋₆alkyl, C₁₋₆haloalkyl, and —OR¹⁰. In some embodiments is a compound of Formula (I″), (I′), (Ia′), (II″), (II′), (IIa′), or (IIb′), or a pharmaceutically acceptable salt or solvate thereof, wherein R² is H. In some embodiments is a compound of Formula (I″), (I′), (Ia′), (II″), (II′), (IIa′), or (IIb′), or a pharmaceutically acceptable salt or solvate thereof, wherein R² is halogen.

Any combination of the groups described above for the various variables is contemplated herein. Throughout the specification, groups and substituents thereof are chosen by one skilled in the field to provide stable moieties and compounds.

In one aspect, described herein is a pharmaceutical composition comprising a compound described herein, or a pharmaceutically acceptable salt or solvate thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by intravenous administration, subcutaneous administration, or oral administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In another aspect, described herein is a method of treating or preventing a liver disease or condition in a mammal, comprising administering to the mammal a compound of Formula (I″), (I′), (Ia′), (II″), (II′), (IIa′), or (IIb′), or a pharmaceutically acceptable salt or solvate thereof. In some embodiments, the liver disease or condition is an alcoholic liver disease or condition. In some embodiments, the liver disease or condition is a nonalcoholic liver disease or condition. In some embodiments, the liver disease or condition is liver inflammation, fatty liver (steatosis), liver fibrosis, hepatitis, cirrhosis, hepatocellular carcinoma, or combinations thereof. In some embodiments, the liver disease or condition is primary biliary cirrhosis, primary sclerosing cholangitis, cholestasis, nonalcoholic steatohepatitis (NASH), nonalcoholic fatty liver disease (NAFLD), or combinations thereof.

In another aspect, described herein is a method of treating a disease or condition in a mammal that would benefit from hydroxysteroid 17β-dehydrogenase 13 (HSD17B13) inhibition comprising administering a compound as described herein, or pharmaceutically acceptable salt or solvate thereof, to the mammal in need thereof. In some embodiments, the disease or condition in a mammal that would benefit from HSD17B13 inhibition is liver inflammation, fatty liver (steatosis), liver fibrosis, hepatitis, cirrhosis, hepatocellular carcinoma, or combinations thereof. In some embodiments, the disease or condition in a mammal that would benefit from HSD17B13 inhibition is primary biliary cirrhosis, primary sclerosing cholangitis, cholestasis, nonalcoholic steatohepatitis (NASH), nonalcoholic fatty liver disease (NAFLD), or combinations thereof.

In another aspect, described herein is a method of modulating hydroxysteroid 17β-dehydrogenase 13 (HSD17B13) activity in a mammal, comprising administering to the mammal a compound of Formula (I″), (I′), (Ia′), (II″), (II′), (IIa′), or (IIb′), or a pharmaceutically acceptable salt or solvate thereof. In some embodiments, modulating comprises inhibiting HSD17B13 activity. In some embodiments of a method of modulating HSD17B13 activity in a mammal, the mammal has a liver disease or condition selected from liver inflammation, fatty liver (steatosis), liver fibrosis, hepatitis, cirrhosis, hepatocellular carcinoma, and combinations thereof. In some embodiments of a method of modulating HSD17B13 activity in a mammal, the mammal has a liver disease or condition selected from primary biliary cirrhosis, primary sclerosing cholangitis, cholestasis, nonalcoholic steatohepatitis (NASH), nonalcoholic fatty liver disease (NAFLD), and combinations thereof.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by inhalation; and/or (e) administered by nasal administration; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the embodiments disclosed herein, the mammal or subject is a human.

In some embodiments, compounds provided herein are administered to a human.

In some embodiments, compounds provided herein are orally administered.

Articles of manufacture, which include packaging material, a compound described herein, or a pharmaceutically acceptable salt thereof, within the packaging material, and a label that indicates that the compound or composition, or pharmaceutically acceptable salt, pharmaceutically active metabolite, pharmaceutically acceptable prodrug, or pharmaceutically acceptable solvate thereof, is used for the treatment, prevention or amelioration of one or more symptoms of a disease or condition that would benefit from HSD17B13 inhibition, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Hydroxysteroid dehydrogenase 17β13 (HSD17b13) is a member of the short-chain dehydrogenase/reductase enzymes highly expressed in the liver on lipid droplets (Horiguchi et al Biochem BiophyslRes Comm, 2008, 370, 235). It has been shown to oxidize retinol, steroids such as estradiol, and bio-active lipids like leukotriene B4 (Abul-Husn et al NEJM, 2018, 378, 1096 and Ma et al Hepatology, 2019, 69 1504). Exosome sequencing analysis of a large patient population identified a minor allele of HSD17b13 (rs72613567:TA) that was associated with reduced odds of developing liver disease (Abul-Husn et al NEJM, 2018, 378, 1096). Relative to subjects with the common HSD17b13 allele (rs72613567:T), subjects with the TA variant have lower serum ALT and AST and lower odds of alcoholic liver disease with or without cirrhosis, nonalcoholic liver disease with or without cirrhosis, and lower odds of hepatocellular carcinoma. Liver pathology analysis reveals that the subjects with the rs72613567:TA allele have decreased odds of having liver pathology analysis classified as NASH vs normal, NASH vs simple steatosis or NASH with fibrosis vs simple steatosis. Liver injury associated with the PNPLA3 rs738409 (p.I148M) is mitigated by the presence of the rs72613567:TA allele of HSD17b13. Additionally hepatic PNPLA3 mRNA expression is decreased in subjects with the rs72613567:TA allele. The rs72613567:TA allele was found to produce a truncated protein which is unable to metabolize substrates such as estradiol, suggesting the hepatic protective effects of the rs72613567:TA allele is due to loss of enzymatic activity.

Patients with NASH have shown elevated expression of hepatic of HSD17b13 mRNA relative to control subject. Further exploration of the role of HSD17b13 in NASH development identified a minor allele rs62305723 that encodes a P260S mutation of HSD17b13 that leads to loss of retinol metabolism and is associated with decreased hepatic ballooning and inflammation (Ma et al Hepatology, 2019, 69 1504).

HSD17b13 rs72613567:TA minor allele is associated with loss of HSD17b13 protein expression in the liver and protection from nonalcoholic steatohepatitis, ballooning degeneration, lobular inflammation and fibrosis. Transcription analysis shows changes in immune-responsive pathways in subjects with rs72613567:TA relative to the major allele (Pirolat et al JLR, 2019, 60, 176).

Subjects with the rs72613567:TA allele of HSD17b13 are not only found to have lower histological evidence of fibrosis, but decreased hepatic expression of fibrotic genes like TGFb2 and Col3a1. In addition loss of HSD17b13 due to the rs72613567:TA allele has been shown to significantly change the expression of inflammatory gene ALOX5 and decreased plasma IL1b, IL6 and IL-10 (Luukkonen et al, JCI, 2020, 5 e132158). HSD17b13 rs72613567:TA carriers also show increased hepatic phospholipids PC(p16:0/16:0), PE(p16:0/18:1), PC(44:5e), PC(36:2e), PE(34:0), PE(36:3) and PC(34:3) possibly due to decreased phospholipid degradation from a decreased hepatic expression of PLD4.

The HSD17b13 rs72613567:TA allele, that has been shown to lack HSD17b13 enzymatic activity, is associated with decreased odds of developing severe fibrosis in patients with chronic HCV infection (About& Abel, NEJM, 2018, 379, 1875). Conversely the major allele rs72613567:T is associated with increasing the risk of development of fibrosis, cirrhosis and HCC in HCV infected patients with the PNPLA3 rs738409:G allele (De Benedittis et al. Gastroenterol Res Pract, 2020, 2020, 4216451).

The loss of function minor allele HSD17b13 rs72613567:TA reduces the risk of developing cirrhosis and hepatocellular carcinoma, is associated with a lower risk of liver-related mortality in the general population and further in patients with cirrhosis (Gellbert-Kristensen et al, Hepatology, 2020, 71, 56). Loss of HSD17b13 function also protects against development of HCC in subjects with alcoholic liver disease (Yang et al, Hepatology, 2019, 70, 231 and Stickel et al, Hepatology, 2020, 72, 88).

PNPLA3 rs738409:G is associated with increased fibrosis in patients with NAFLD. The minor HSD17b13 rs72613567:TA allele has been shown to counteract the PNPLA3 rs738409:G allele and decrease the prevalence of severe inflammation, ballooning and fibrosis (Seko et al, Liver Int, 2020, 40, 1686).

Loss of HSD17b13 enzymatic activity due to carrying the rs72613567:TA allele may delay the onset of autoimmune hepatitis (Mederacke et al, Aliment Pharmacol Ther, 2020, 00, 1).

HSD17b13 rs72613567:TA allele is associated with decreased fibrosis and cirrhosis in patents with copper induced liver injury from Wilson's disease (Ferenci et al, 2019, JHEP, 1, 2).

### Compounds

Compounds described herein, including pharmaceutically acceptable salts, prodrugs, active metabolites and pharmaceutically acceptable solvates thereof, are HSD17B13 inhibitors.

In some embodiments is a compound of Formula (I″), or a pharmaceutically acceptable salt or solvate thereof:

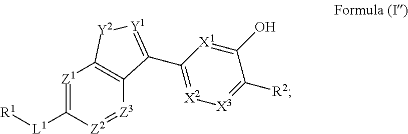

wherein:
X¹, X², and X³ are each independently CR³ or N;
Y¹ is CR⁴ or N;
Y² is N(R⁹), O, or C(R⁴)₂;
Z¹, Z², and Z³ are each independently CR⁵ or N;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₁₀cycloalkyl and C₂₋₉heterocycloalkyl, wherein C₃₋₁₀cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉ heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R³, each R⁴, and each R⁵ are each independently selected from H, halogen, —CN, C₁₋₆ alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁷ are each independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹⁴, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
R⁹ is selected from H, C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆ cycloalkyl, C₂₋₉heterocycloalkyl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl; and
each R¹⁴ is independently selected C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆ cycloalkyl, C₂₋₉heterocycloalkyl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, and C₁₋₉heteroaryl.

In some embodiments is a compound of Formula (I′), or a pharmaceutically acceptable salt or solvate thereof:

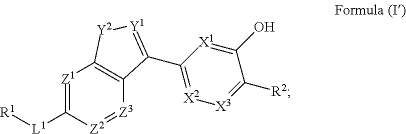

wherein:
X¹, X², and X³ are each independently CR³ or N;
Y¹ is CR⁴ or N;
Y² is N(R⁹), O, or C(R⁴)₂;
Z¹, Z², and Z³ are each independently CR⁵ or N;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R³, each R⁴, and each R⁵ are each independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁷ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹⁴, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁹ is selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl; and
each R¹⁴ is independently selected C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆ cycloalkyl, C₂₋₉heterocycloalkyl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, and C₁₋₉heteroaryl.

In some embodiments is a compound of Formula (I), or a pharmaceutically acceptable salt or solvate thereof:

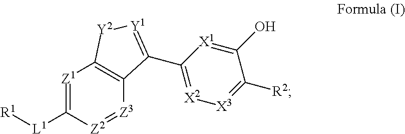

wherein:
X¹, X², and X³ are each independently CR³ or N;
Y¹ is CR⁴ or N;
Y² is N(R⁹), O, or C(R⁴)₂;
Z¹, Z², and Z³ are each independently CR⁵ or N;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R³, each R⁴, and each R⁵ are each independently selected from H, halogen, —CN, C₁₋₆ alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ and each R⁷ are each independently selected from halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁹ is selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl.

In some embodiments is a compound of Formula (I″), (I′), or (I), or a pharmaceutically acceptable salt or solvate thereof, wherein X¹, X², and X³ are each CR³. In some embodiments is a compound of Formula (I″), (I′), or (I), or a pharmaceutically acceptable salt or solvate thereof, wherein X¹, X², and X³ are each CR³ and each R³ is independently selected from H, halogen, C₁₋₆alkyl, C₁₋₆haloalkyl, and —OR¹⁰. In some embodiments is a compound of Formula (I″), (I′), or (I), or a pharmaceutically acceptable salt or solvate thereof, wherein X¹, X², and X³ are each CR³ and each R³ is independently selected from H, halogen, C₁₋₆alkyl, and C₁₋₆haloalkyl. In some embodiments is a compound of Formula (I″), (I′), or (I), or a pharmaceutically acceptable salt or solvate thereof, wherein X¹, X², and X³ are each CR³ and each R³ is independently selected from H, halogen, and C₁₋₆ haloalkyl. In some embodiments is a compound of Formula (I″), (I′), or (I), or a pharmaceutically acceptable salt or solvate thereof, wherein X¹ is C(H), X² is C(H), and X³ is C(CF₃). In some embodiments is a compound of Formula (I″), (I′), or (I), or a pharmaceutically acceptable salt or solvate thereof, wherein X¹ is C(F), X² is C(H), and X³ is C(CF₃). In some embodiments is a compound of Formula (I″), (I′), or (I), or a pharmaceutically acceptable salt or solvate thereof, wherein X¹ is C(Cl), X² is C(H), and X³ is C(CF₃). In some embodiments is a compound of Formula (I″), (I′), or (I), or a pharmaceutically acceptable salt or solvate thereof, wherein X¹ is C(H), X² is C(H), and X³ is C(F). In some embodiments is a compound of Formula (I″), (I′), or (I), or a pharmaceutically acceptable salt or solvate thereof, wherein X¹ is C(H), X² is C(H), and X³ is C(Cl).

In some embodiments is a compound of Formula (I″), (I′), or (I), or a pharmaceutically acceptable salt or solvate thereof, wherein R² is selected from H, halogen, C₁₋₆alkyl, C₁₋₆haloalkyl, and —OR¹⁰. In some embodiments is a compound of Formula (I″), (I′), or (I), or a pharmaceutically acceptable salt or solvate thereof, wherein R² is H. In some embodiments is a compound of Formula (I″), (I′), or (I), or a pharmaceutically acceptable salt or solvate thereof, wherein R² is halogen. In some embodiments is a compound of Formula (I″), (I′), or (I), or a pharmaceutically acceptable salt or solvate thereof, wherein R² is F. In some embodiments is a compound of Formula (I″), (I′), or (I), or a pharmaceutically acceptable salt or solvate thereof, wherein R² is Cl. In some embodiments is a compound of Formula (I″), (I′), or (I), or a pharmaceutically acceptable salt or solvate thereof, wherein R² is C₁₋₆alkyl. In some embodiments is a compound of Formula (I″), (I′), or (I), or a pharmaceutically acceptable salt or solvate thereof, wherein R² is C₁₋₆haloalkyl. In some embodiments is a compound of Formula (I″), (I′), or (I), or a pharmaceutically acceptable salt or solvate thereof, wherein R² is —OR¹⁰. In some embodiments is a compound of Formula (I″), (I′), or (I), or a pharmaceutically acceptable salt or solvate thereof, wherein R² is —OH. In some embodiments is a compound of Formula (I″), (I′), or (I), or a pharmaceutically acceptable salt or solvate thereof, wherein R² is —OCH₃.

In some embodiments is a compound of Formula (I″), (I′), or (I), or a pharmaceutically acceptable salt or solvate thereof, wherein Y¹ is N and Y² is CR⁴. In some embodiments is a compound of Formula (I″), (I′), or (I), or a pharmaceutically acceptable salt or solvate thereof, wherein Y¹ is CR⁴ and Y² is CR⁴. In some embodiments is a compound of Formula (I″), (I′), or (I), or a pharmaceutically acceptable salt or solvate thereof, wherein Y¹ is CR⁴ and Y² is N. In some embodiments is a compound of Formula (I″), (I′), or (I), or a pharmaceutically acceptable salt or solvate thereof, wherein each R⁴ is independently selected from H, halogen, C₁₋₆alkyl, and C₃₋₆cycloalkyl. In some embodiments is a compound of Formula (I″), (I′), or (I), or a pharmaceutically acceptable salt or solvate thereof, wherein Y¹ is N and Y² is C(H). In some embodiments is a compound of Formula (I″), (I′), or (I), or a pharmaceutically acceptable salt or solvate thereof, wherein Y¹ is C(H) and Y² is C(H). In some embodiments is a compound of Formula (I″), (I′), or (I), or a pharmaceutically acceptable salt or solvate thereof, wherein Y¹ is C(H) and Y² is N. In some embodiments is a compound of Formula (I″), (I′), or (I), or a pharmaceutically acceptable salt or solvate thereof, wherein Y¹ is N and Y² is N.

In some embodiments is a compound of Formula (I″), (I′), or (I), or a pharmaceutically acceptable salt or solvate thereof, wherein Z¹, Z², and Z³ are CR⁵. In some embodiments is a compound of Formula (I″), (I′), or (I), or a pharmaceutically acceptable salt or solvate thereof, wherein Z¹ is N; and Z² and Z³ are CR⁵. In some embodiments is a compound of Formula (I″), (I′), or (I), or a pharmaceutically acceptable salt or solvate thereof, wherein Z² is N; and Z¹ and Z³ are CR⁵. In some embodiments is a compound of Formula (I″), (I′), or (I), or a pharmaceutically acceptable salt or solvate thereof, wherein Z³ is N; and Z¹ and Z² are CR⁵. In some embodiments is a compound of Formula (I″), (I′), or (I), or a pharmaceutically acceptable salt or solvate thereof, wherein Z¹ is CR⁵; and Z² and Z³ are N. In some embodiments is a compound of Formula (I″), (I′), or (I), or a pharmaceutically acceptable salt or solvate thereof, wherein Z² is CR⁵; and Z¹ and Z³ are N. In some embodiments is a compound of Formula (I″), (I′), or (I), or a pharmaceutically acceptable salt or solvate thereof, wherein Z³ is CR⁵; and Z¹ and Z² are N. In some embodiments is a compound of Formula (I″), (I′), or (I), or a pharmaceutically acceptable salt or solvate thereof, wherein each R⁵ is independently selected from H, halogen, C₁₋₆alkyl, and —OR¹⁰. In some embodiments is a compound of Formula (I″), (I′), or (I), or a pharmaceutically acceptable salt or solvate thereof, wherein each R⁵ is H. In some embodiments is a compound of Formula (I″), (I′), or (I), or a pharmaceutically acceptable salt or solvate thereof, wherein Z¹, Z², and Z³ are C(H). In some embodiments is a compound of Formula (I″), (I′), or (I), or a pharmaceutically acceptable salt or solvate thereof, wherein Z¹ is N; and Z² and Z³ are C(H). In some embodiments is a compound of Formula (I″), (I′), or (I), or a pharmaceutically acceptable salt or solvate thereof, wherein Z² is N; and Z¹ and Z³ are C(H). In some embodiments is a compound of Formula (I″), (I′), or (I), or a pharmaceutically acceptable salt or solvate thereof, wherein Z³ is N; and Z¹ and Z² are C(H). In some embodiments is a compound of Formula (I″), (I′), or (I), or a pharmaceutically acceptable salt or solvate thereof, wherein Z¹ is C(H); and Z² and Z³ are N. In some embodiments is a compound of Formula (I″), (I′), or (I), or a pharmaceutically acceptable salt or solvate thereof, wherein Z² is C(H); and Z¹ and Z³ are N. In some embodiments is a compound of Formula (I″), (I′), or (I), or a pharmaceutically acceptable salt or solvate thereof, wherein Z³ is C(H); and Z¹ and Z² are N.

In some embodiments is a compound of Formula (I″), (I′), or (I), or a pharmaceutically acceptable salt or solvate thereof, wherein L¹ is a bond. In some embodiments is a compound of Formula (I″), (I′), or (I), or a pharmaceutically acceptable salt or solvate thereof, wherein L¹ is —O—. In some embodiments is a compound of Formula (I″), (I′), or (I), or a pharmaceutically acceptable salt or solvate thereof, wherein L¹ is —N(R¹⁰)—. In some embodiments is a compound of Formula (I″), (I′), or (I), or a pharmaceutically acceptable salt or solvate thereof, wherein L¹ is —N(H)—. In some embodiments is a compound of Formula (I″), (I′), or (I), or a pharmaceutically acceptable salt or solvate thereof, wherein L¹ is —C(R¹⁰)(R¹¹)N(R¹⁰)—. In some embodiments is a compound of Formula (I″), (I′), or (I), or a pharmaceutically acceptable salt or solvate thereof, wherein L¹ is —CH₂N(H)—. In some embodiments is a compound of Formula (I″), (I′), or (I), or a pharmaceutically acceptable salt or solvate thereof, wherein L¹ is —N(R¹⁰)C(R¹⁰)(R¹¹)—. In some embodiments is a compound of Formula (I″), (I′), or (I), or a pharmaceutically acceptable salt or solvate thereof, wherein L¹ is —N(H)CH₂—.

In some embodiments is a compound of Formula (I″), (I′), or (I), or a pharmaceutically acceptable salt or solvate thereof, wherein R¹ is selected from C₃₋₈ cycloalkyl and C₂₋₉heterocycloalkyl, wherein C₃₋₈cycloalkyl and C₂₋₉heterocycloalkyl are optionally substituted with one, two, or three R⁶. In some embodiments is a compound of Formula (I″), (I′), or (I), or a pharmaceutically acceptable salt or solvate thereof, wherein R¹ is C₂₋₉heterocycloalkyl optionally substituted with one, two, or three R⁶. In some embodiments is a compound of Formula (I″), (I′), or (I), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl, wherein piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl are optionally substituted with one, two, or three R⁶. In some embodiments is a compound of Formula (I″), (I′), or (I), or a pharmaceutically acceptable

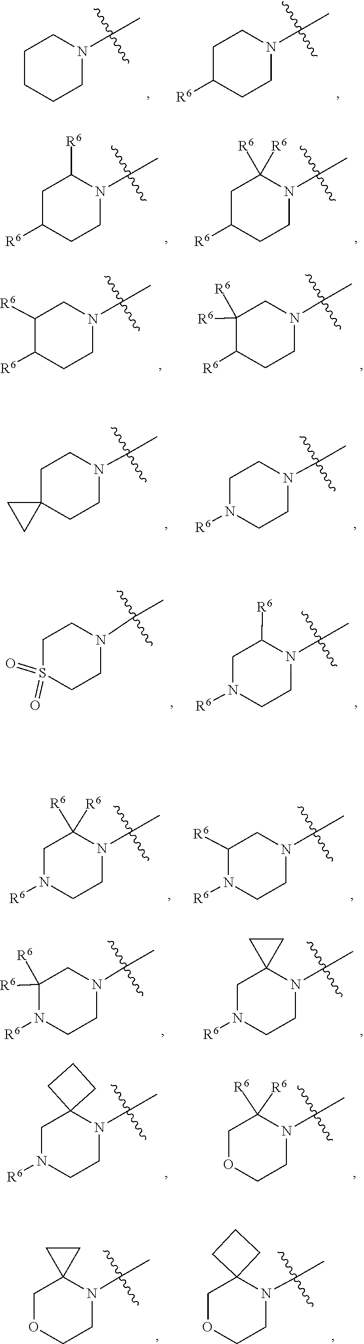

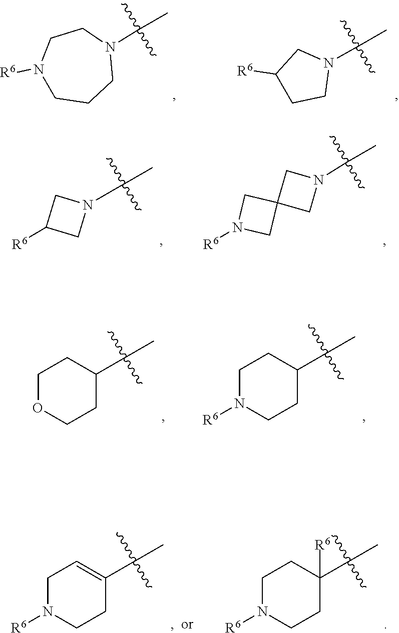

In some embodiments is a compound of Formula (I″), (I′), or (I), or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (I″), (I′), or (I), or a pharmaceutically acceptable salt or solvate thereof, wherein R¹ is

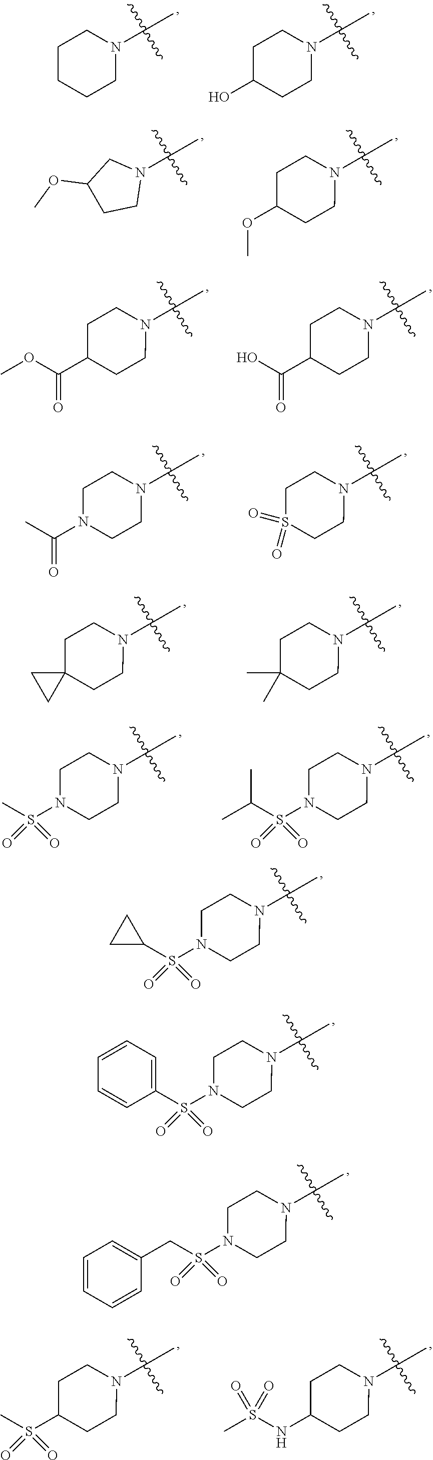

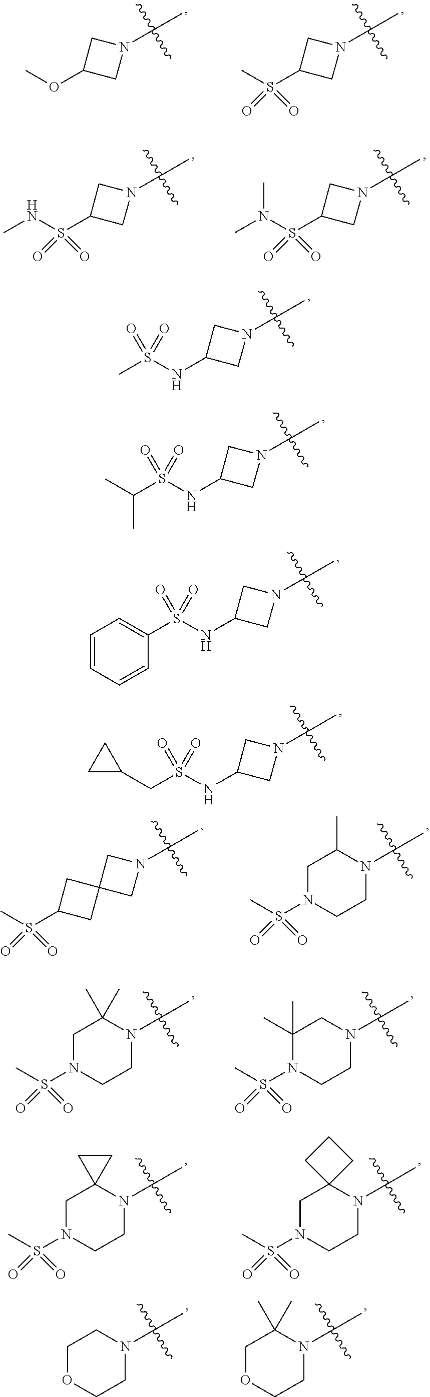

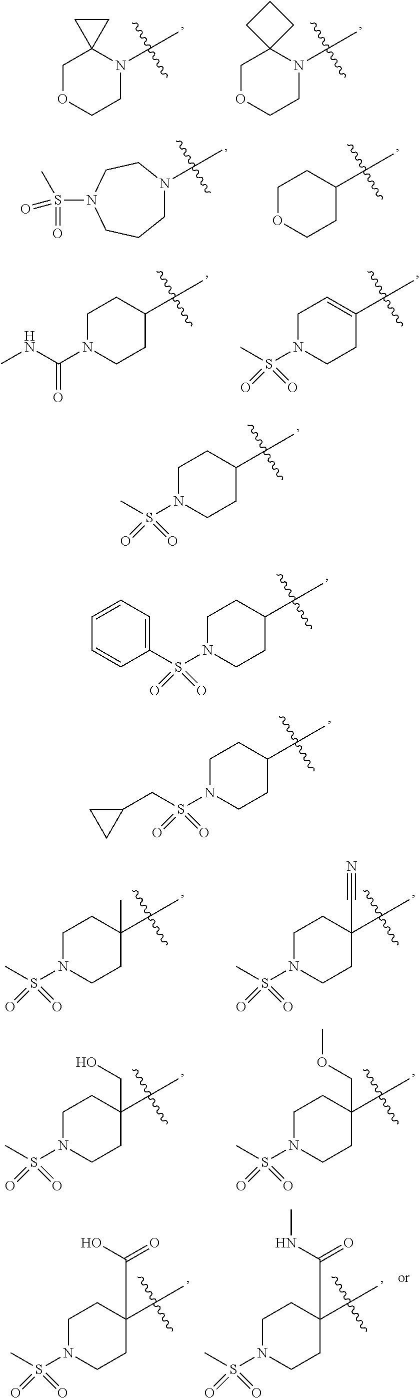

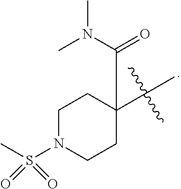 [SMILES: *C1(C(=O)N(C)C)CCN(S(C)(=O)=O)CC1]

In some embodiments is a compound of Formula (I″), (I′), or (I), or a pharmaceutically acceptable salt or solvate thereof, wherein R¹ is

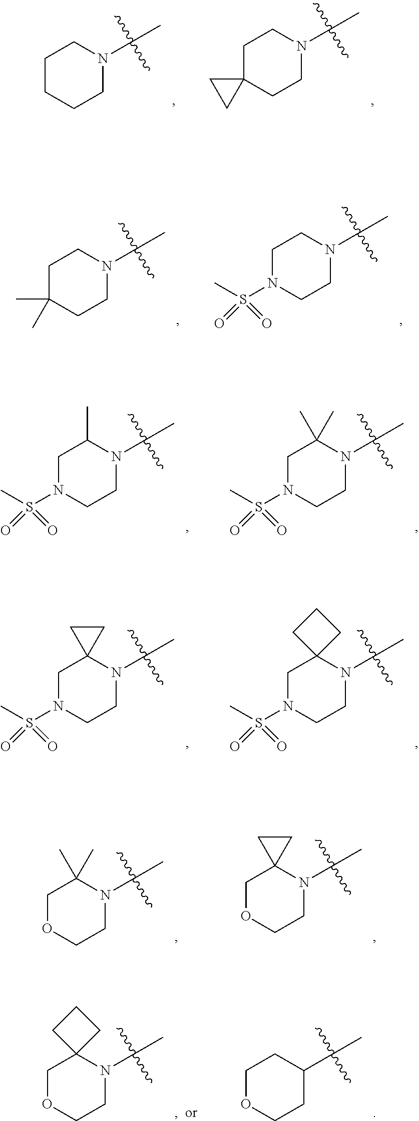

In some embodiments is a compound of Formula (I″), (I′), or (I), or a pharmaceutically acceptable salt or solvate thereof, wherein R¹ is

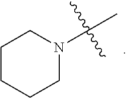 [SMILES: *N1CCCCC1]

In some embodiments is a compound of Formula (I″), (I′), or (I), or a pharmaceutically acceptable salt or solvate thereof, wherein R¹ is

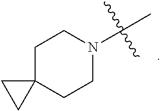 [SMILES: *N1CCC2(CC1)CC2]

In some embodiments is a compound of Formula (I″), (I′), or (I), or a pharmaceutically acceptable salt or solvate thereof, wherein R¹ is

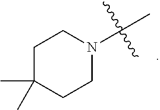 [SMILES: *N1CCC(C)(C)CC1]

In some embodiments is a compound of Formula (I″), (I′), or (I), or a pharmaceutically acceptable salt or solvate thereof, wherein R¹ is

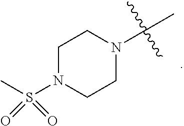 [SMILES: *N1CCN(S(C)(=O)=O)CC1]

In some embodiments is a compound of Formula (I″), (I′), or (I), or a pharmaceutically acceptable salt or solvate thereof, wherein R¹ is

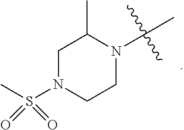 [SMILES: *N1CCN(S(C)(=O)=O)CC1C]

In some embodiments is a compound of Formula (I″), (I′), or (I), or a pharmaceutically acceptable salt or solvate thereof, wherein R¹ is

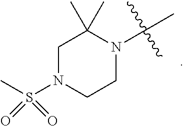 [SMILES: *N1CCN(S(C)(=O)=O)CC1(C)C]

In some embodiments is a compound of Formula (I″), (I′), or (I), or a pharmaceutically acceptable salt or solvate thereof, wherein R¹ is

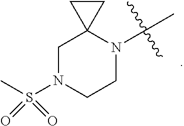 [SMILES: *N1CCN(S(C)(=O)=O)CC12CC2]

In some embodiments is a compound of Formula (I″), (I′), or (I), or a pharmaceutically acceptable salt or solvate thereof, wherein R¹ is

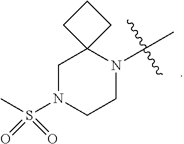 [SMILES: *N1CCN(S(C)(=O)=O)CC12CCC2]

In some embodiments is a compound of Formula (I″), (I′), or (I), or a pharmaceutically acceptable salt or solvate thereof, wherein R¹ is

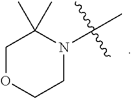 [SMILES: *N1CCOCC1(C)C]

In some embodiments is a compound of Formula (I″), (I′), or (I), or a pharmaceutically acceptable salt or solvate thereof, wherein R¹ is

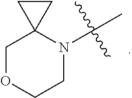 [SMILES: *N1CCOCC12CC2]

In some embodiments is a compound of Formula (I″), (I′), or (I), or a pharmaceutically acceptable salt or solvate thereof, wherein R¹ is

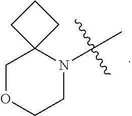 [SMILES: *N1CCOCC12CCC2]

In some embodiments is a compound of Formula (I″), (I′), or (I), or a pharmaceutically acceptable salt or solvate thereof, wherein R¹ is

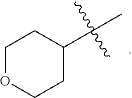 [SMILES: *C1CCOCC1]

In some embodiments is a compound of Formula (I″), (I′), or (I), or a pharmaceutically acceptable salt or solvate thereof, wherein R¹ is C₃₋₈cycloalkyl optionally substituted with one, two, or three R⁶.

In some embodiments is a compound of Formula (I″), (I′), or (I), or a pharmaceutically acceptable salt or solvate thereof, wherein R¹ is C₁₋₉heteroaryl substituted with one, two, or three R⁷. In some embodiments is a compound of Formula (I″), (I′), or (I), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷. In some embodiments is a compound of Formula (I″), (I′), or (I), or a pharmaceutically acceptable salt or solvate thereof, wherein R¹ is

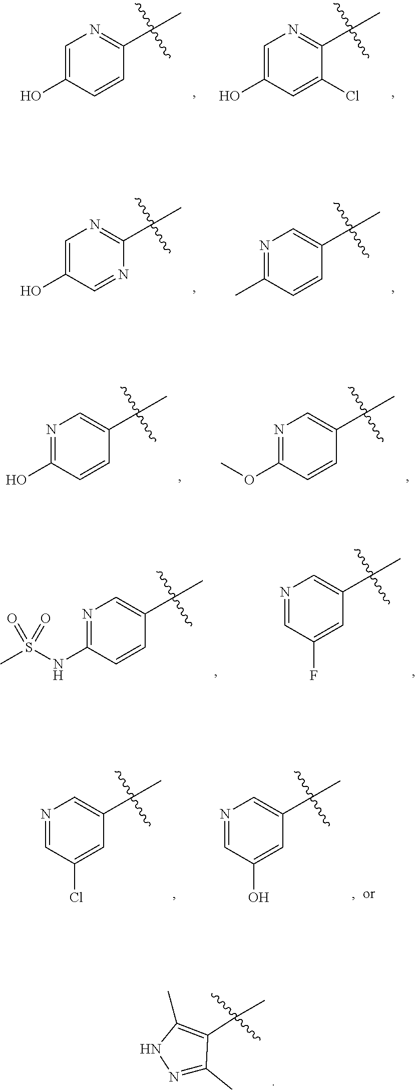

In some embodiments is a compound of Formula (Ia′), or a pharmaceutically acceptable salt or solvate thereof:

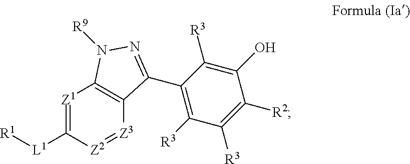

wherein:
Z¹, Z², and Z³ are each independently CR⁵ or N;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R³ and each R⁵ are each independently selected from H, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁷ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹⁴, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁹ is selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl; and
each R¹⁴ is independently selected C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆ cycloalkyl, C₂₋₉heterocycloalkyl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, and C₁₋₉heteroaryl.

In some embodiments is a compound of Formula (Ia′), or a pharmaceutically acceptable salt or solvate thereof, wherein each R³ is independently selected from H, halogen, C₁₋₆alkyl, C₁₋₆haloalkyl, and —OR¹⁰. In some embodiments is a compound of Formula (Ia′), or a pharmaceutically acceptable salt or solvate thereof, wherein each R³ is independently selected from H, halogen, C₁₋₆alkyl, and C₁₋₆haloalkyl. In some embodiments is a compound of Formula (Ia′), or a pharmaceutically acceptable salt or solvate thereof, wherein each R³ is independently selected from H, halogen, and C₁₋₆haloalkyl. In some embodiments is a compound of Formula (Ia′), or a pharmaceutically acceptable salt or solvate thereof, wherein each R³ is independently selected from H and C₁₋₆haloalkyl.

In some embodiments is a compound of Formula (Ia′), or a pharmaceutically acceptable salt or solvate thereof, wherein R² is selected from H, halogen, C₁₋₆alkyl, C₁₋₆ haloalkyl, and —OR¹⁰. In some embodiments is a compound of Formula (Ia′), or a pharmaceutically acceptable salt or solvate thereof, wherein R² is H. In some embodiments is a compound of Formula (Ia′), or a pharmaceutically acceptable salt or solvate thereof, wherein R² is halogen. In some embodiments is a compound of Formula (Ia′), or a pharmaceutically acceptable salt or solvate thereof, wherein R² is F. In some embodiments is a compound of Formula (Ia′), or a pharmaceutically acceptable salt or solvate thereof, wherein R² is Cl. In some embodiments is a compound of Formula (Ia′), or a pharmaceutically acceptable salt or solvate thereof, wherein R² is C₁₋₆alkyl. In some embodiments is a compound of Formula (Ia′), or a pharmaceutically acceptable salt or solvate thereof, wherein R² is C₁₋₆haloalkyl. In some embodiments is a compound of Formula (Ia′), or a pharmaceutically acceptable salt or solvate thereof, wherein R² is —OR¹⁰. In some embodiments is a compound of Formula (Ia′), or a pharmaceutically acceptable salt or solvate thereof, wherein R² is —OH. In some embodiments is a compound of Formula (Ia′), or a pharmaceutically acceptable salt or solvate thereof, wherein R² is —OCH₃.

In some embodiments is a compound of Formula (Ia′), or a pharmaceutically acceptable salt or solvate thereof, wherein Z¹, Z², and Z³ are CR⁵. In some embodiments is a compound of Formula (Ia′), or a pharmaceutically acceptable salt or solvate thereof, wherein Z¹ is N; and Z² and Z³ are CR⁵. In some embodiments is a compound of Formula (Ia′), or a pharmaceutically acceptable salt or solvate thereof, wherein Z² is N; and Z¹ and Z³ are CR⁵. In some embodiments is a compound of Formula (Ia′), or a pharmaceutically acceptable salt or solvate thereof, wherein Z³ is N; and Z¹ and Z² are CR⁵. In some embodiments is a compound of Formula (Ia′), or a pharmaceutically acceptable salt or solvate thereof, wherein Z¹ is CR⁵; and Z² and Z³ are N. In some embodiments is a compound of Formula (Ia′), or a pharmaceutically acceptable salt or solvate thereof, wherein Z² is CR⁵; and Z¹ and Z³ are N. In some embodiments is a compound of Formula (Ia′), or a pharmaceutically acceptable salt or solvate thereof, wherein Z³ is CR⁵; and Z¹ and Z² are N. In some embodiments is a compound of Formula (Ia′), or a pharmaceutically acceptable salt or solvate thereof, wherein each R⁵ is independently selected from H, halogen, C₁₋₆alkyl, and —OR¹⁰. In some embodiments is a compound of Formula (Ia′), or a pharmaceutically acceptable salt or solvate thereof, wherein each R⁵ is H. In some embodiments is a compound of Formula (Ia′), or a pharmaceutically acceptable salt or solvate thereof, wherein Z¹, Z², and Z³ are C(H). In some embodiments is a compound of Formula (Ia′), or a pharmaceutically acceptable salt or solvate thereof, wherein Z¹ is N; and Z² and Z³ are C(H). In some embodiments is a compound of Formula (Ia′), or a pharmaceutically acceptable salt or solvate thereof, wherein Z² is N; and Z¹ and Z³ are C(H). In some embodiments is a compound of Formula (Ia′), or a pharmaceutically acceptable salt or solvate thereof, wherein Z³ is N; and Z¹ and Z² are C(H). In some embodiments is a compound of Formula (Ia′), or a pharmaceutically acceptable salt or solvate thereof, wherein Z¹ is C(H); and Z² and Z³ are N. In some embodiments is a compound of Formula (Ia′), or a pharmaceutically acceptable salt or solvate thereof, wherein Z² is C(H); and Z¹ and Z³ are N. In some embodiments is a compound of Formula (Ia′), or a pharmaceutically acceptable salt or solvate thereof, wherein Z³ is C(H); and Z¹ and Z² are N.

In some embodiments is a compound of Formula (Ia′), or a pharmaceutically acceptable salt or solvate thereof, wherein L¹ is a bond. In some embodiments is a compound of Formula (Ia′), or a pharmaceutically acceptable salt or solvate thereof, wherein L¹ is —O—. In some embodiments is a compound of Formula (Ia′), or a pharmaceutically acceptable salt or solvate thereof, wherein L¹ is —N(R¹⁰)—. In some embodiments is a compound of Formula (Ia′), or a pharmaceutically acceptable salt or solvate thereof, wherein L¹ is —N(H)—. In some embodiments is a compound of Formula (Ia′), or a pharmaceutically acceptable salt or solvate thereof, wherein L¹ is —C(R¹⁰)(R¹¹)N(R¹⁰)—. In some embodiments is a compound of Formula (Ia′), or a pharmaceutically acceptable salt or solvate thereof, wherein L¹ is —CH₂N(H)—. In some embodiments is a compound of Formula (Ia′), or a pharmaceutically acceptable salt or solvate thereof, wherein L¹ is —N(R¹⁰)C(R¹⁰)(R¹¹)—. In some embodiments is a compound of Formula (Ia′), or a pharmaceutically acceptable salt or solvate thereof, wherein L¹ is —N(H)CH₂—.

In some embodiments is a compound of Formula (Ia′), or a pharmaceutically acceptable salt or solvate thereof, wherein R¹ is selected from C₃₋₈cycloalkyl and C₂₋₉heterocycloalkyl, wherein C₃₋₈cycloalkyl and C₂₋₉heterocycloalkyl are optionally substituted with one, two, or three R⁶. In some embodiments is a compound of Formula (Ia′), or a pharmaceutically acceptable salt or solvate thereof, wherein R¹ is C₂₋₉heterocycloalkyl optionally substituted with one, two, or three R⁶. In some embodiments is a compound of Formula (Ia′), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl, wherein piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl are optionally substituted with one, two, or three R⁶. In some embodiments is a compound of Formula (Ia′), or a pharmaceutically acceptable salt or solvate thereof, wherein R¹ is

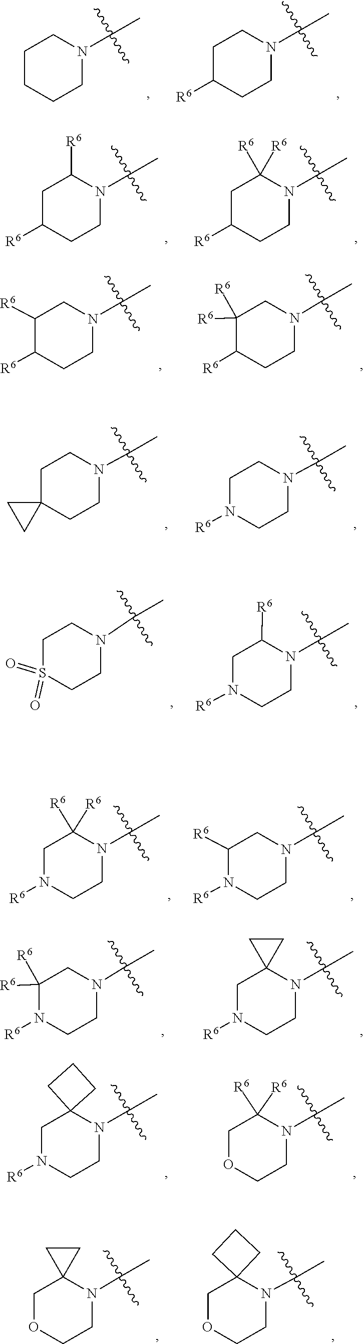

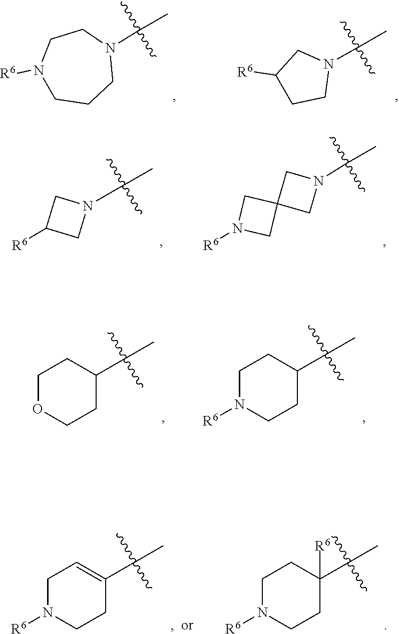

In some embodiments is a compound of Formula (Ia′), or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (Ia′), or a pharmaceutically acceptable salt or solvate thereof, wherein R¹ is

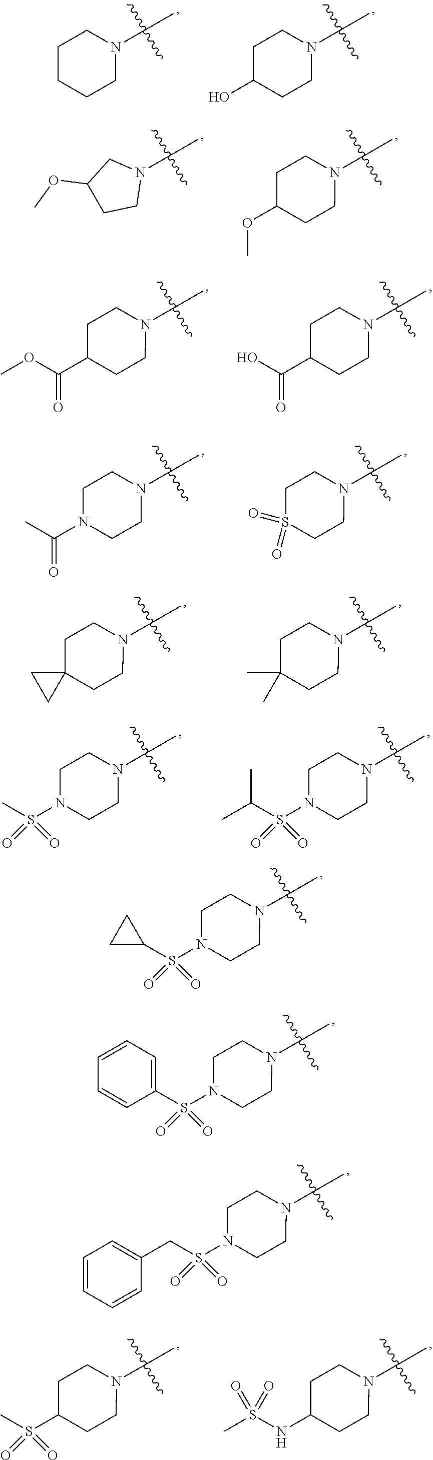

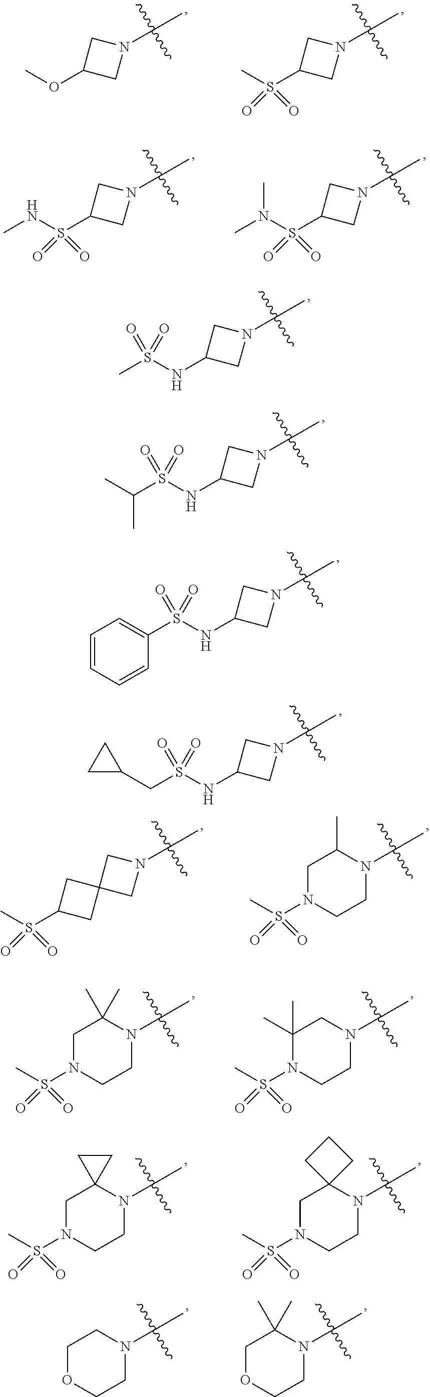

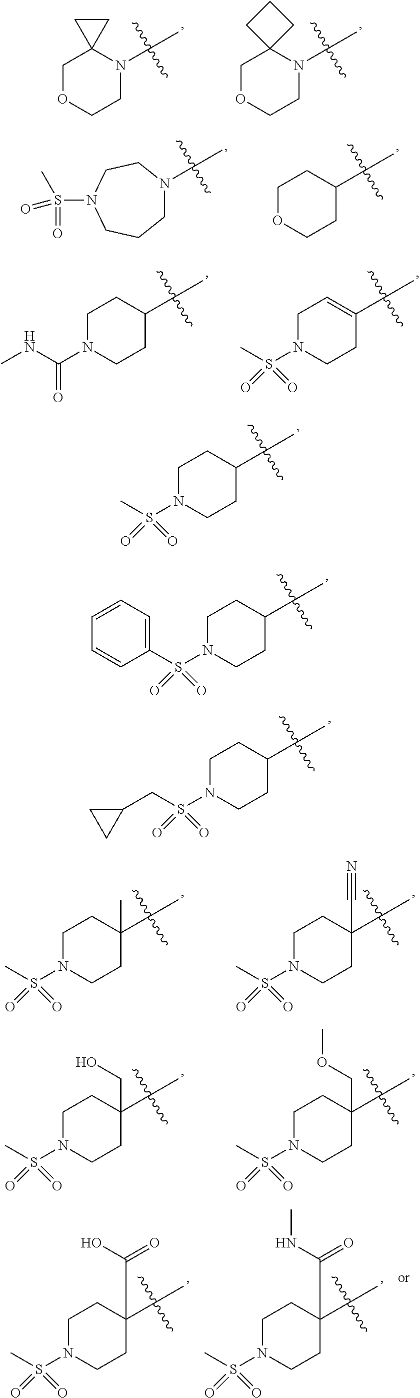

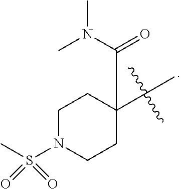 [SMILES: *C1(C(=O)N(C)C)CCN(S(C)(=O)=O)CC1]

In some embodiments is a compound of Formula (Ia′), or a pharmaceutically acceptable salt or solvate thereof, wherein R¹ is

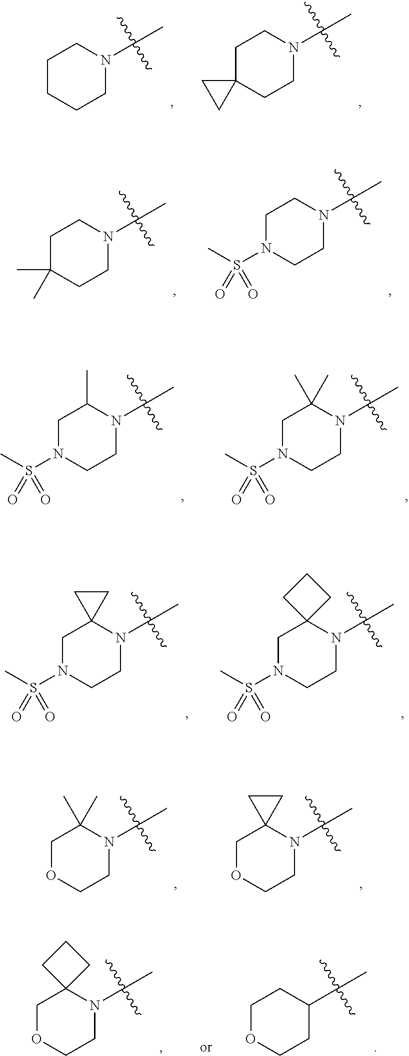

In some embodiments is a compound of Formula (Ia′), or a pharmaceutically acceptable salt or solvate thereof, wherein R¹ is

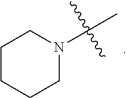 [SMILES: *N1CCCCC1]

In some embodiments is a compound of Formula (Ia′), or a pharmaceutically acceptable salt or solvate thereof, wherein R¹ is

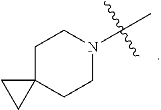 [SMILES: *N1CCC2(CC1)CC2]

In some embodiments is a compound of Formula (Ia′), or a pharmaceutically acceptable salt or solvate thereof, wherein R¹ is

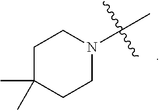 [SMILES: *N1CCC(C)(C)CC1]

In some embodiments is a compound of Formula (Ia′) or a pharmaceutically acceptable salt or solvate thereof, wherein R¹ is

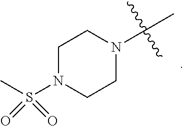 [SMILES: *N1CCN(S(C)(=O)=O)CC1]

In some embodiments is a compound of Formula (Ia′), or a pharmaceutically acceptable salt or solvate thereof, wherein R¹ is

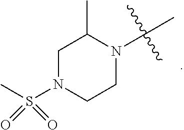 [SMILES: *N1CCN(S(C)(=O)=O)CC1C]

In some embodiments is a compound of Formula (Ia′), or a pharmaceutically acceptable salt or solvate thereof, wherein R¹ is

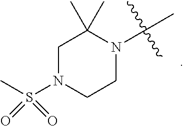 [SMILES: *N1CCN(S(C)(=O)=O)CC1(C)C]

In some embodiments is a compound of Formula (Ia′), or a pharmaceutically acceptable salt or solvate thereof, wherein R¹ is

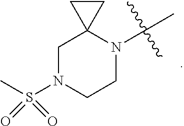 [SMILES: *N1CCN(S(C)(=O)=O)CC12CC2]

In some embodiments is a compound of Formula (Ia′), or a pharmaceutically acceptable salt or solvate thereof, wherein R¹ is

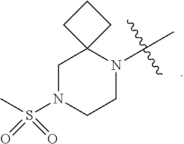 [SMILES: *N1CCN(S(C)(=O)=O)CC12CCC2]

In some embodiments is a compound of Formula (Ia′), or a pharmaceutically acceptable salt or solvate thereof, wherein R¹ is

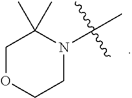 [SMILES: *N1CCOCC1(C)C]

In some embodiments is a compound of Formula (Ia′), or a pharmaceutically acceptable salt or solvate thereof, wherein R¹ is

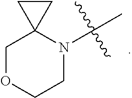 [SMILES: *N1CCOCC12CC2]

In some embodiments is a compound of Formula (Ia′), or a pharmaceutically acceptable salt or solvate thereof, wherein R¹ is

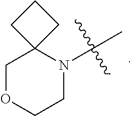 [SMILES: *N1CCOCC12CCC2]

In some embodiments is a compound of Formula (Ia′), or a pharmaceutically acceptable salt or solvate thereof, wherein R¹ is

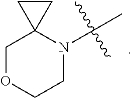 [SMILES: *N1CCOCC12CC2]

In some embodiments is a compound of Formula (Ia′), or a pharmaceutically acceptable salt or solvate thereof, wherein R¹ is

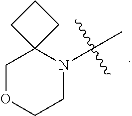 [SMILES: *N1CCOCC12CCC2]

In some embodiments is a compound of Formula (Ia′), or a pharmaceutically acceptable salt or solvate thereof, wherein R¹ is

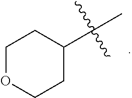 [SMILES: *C1CCOCC1]

In some embodiments is a compound of Formula (Ia′), or a pharmaceutically acceptable salt or solvate thereof, wherein R¹ is C₃₋₈cycloalkyl optionally substituted with one, two, or three R⁶.

In some embodiments is a compound of Formula (Ia′), or a pharmaceutically acceptable salt or solvate thereof, wherein R¹ is C₁₋₉heteroaryl substituted with one, two, or three R⁷. In some embodiments is a compound of Formula (Ia′), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷. In some embodiments is a compound of Formula (Ia′), or a pharmaceutically acceptable salt or solvate thereof, wherein R¹ is

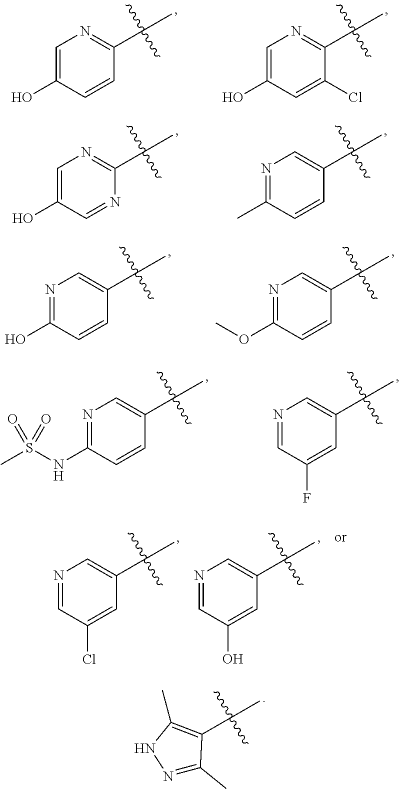

In some embodiments is a compound of Formula (II″), or a pharmaceutically acceptable salt or solvate thereof:

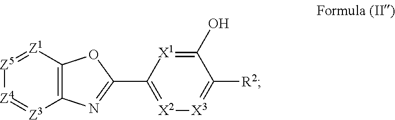

wherein:
X¹, X², and X³ are each independently CR³ or N;
Z¹ and Z³ are each independently CR⁵ or N;
Z⁴ and Z⁵ are each independently CR⁵, CR⁸, or N, wherein one of Z⁴ and Z⁵ is CR⁸;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₁₀cycloalkyl and C₂₋₉heterocycloalkyl, wherein C₃₋₁₀cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R³ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁵ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁷ are each independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
R⁸ is -L¹-R¹;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl.

In some embodiments is a compound of Formula (II′), or a pharmaceutically acceptable salt or solvate thereof:

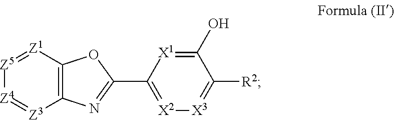

wherein:
X¹, X², and X³ are each independently CR³ or N;
Z¹ and Z³ are each independently CR⁵ or N;
Z⁴ and Z⁵ are each independently CR⁵, CR⁸, or N, wherein one of Z⁴ and Z⁵ is CR⁸;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R³ and each R⁵ are each independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁷ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹⁴, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁸ is -L¹-R¹;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
each R¹⁴ is independently selected C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, C₁₋₆alkyl, C₁₋₆ haloalkyl, C₁₋₆alkoxy, C₃₋₆cycloalkyl, C₂₋₉heterocycloalkyl, and C₁₋₉heteroaryl.

In some embodiments is a compound of Formula (II), or a pharmaceutically acceptable salt or solvate thereof:

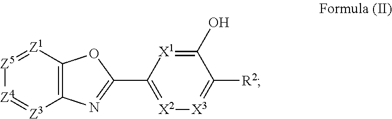

wherein:
X¹, X², and X³ are each independently CR³ or N;
Z¹ and Z³ are each independently CR⁵ or N;
Z⁴ and Z⁵ are each independently CR⁵, CR⁸, or N, wherein one of Z⁴ and Z⁵ is CR⁸;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R³ and each R⁵ are each independently selected from H, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ and each R⁷ are each independently selected from halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁸ is -L¹-R¹;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl.

In some embodiments is a compound of Formula (II″), (II′), or (II), or a pharmaceutically acceptable salt or solvate thereof, wherein X¹, X², and X³ are each CR³. In some embodiments is a compound of Formula (II″), (II′), or (II), or a pharmaceutically acceptable salt or solvate thereof, wherein X¹, X², and X³ are each CR³ and each R³ is independently selected from H, halogen, C₁₋₆alkyl, C₁₋₆haloalkyl, and —OR¹⁰. In some embodiments is a compound of Formula (II″), (II′), or (II), or a pharmaceutically acceptable salt or solvate thereof, wherein X¹, X², and X³ are each CR³ and each R³ is independently selected from H, halogen, C₁₋₆alkyl, and C₁₋₆haloalkyl. In some embodiments is a compound of Formula (II″), (II′), or (II), or a pharmaceutically acceptable salt or solvate thereof, wherein X¹, X², and X³ are each CR³ and each R³ is independently selected from H, halogen, and C₁₋₆haloalkyl. In some embodiments is a compound of Formula (II″), (II′), or (II), or a pharmaceutically acceptable salt or solvate thereof, wherein X¹ is C(H), X² is C(H), and X³ is C(CF₃). In some embodiments is a compound of Formula (II″), (II′), or (II), or a pharmaceutically acceptable salt or solvate thereof, wherein X¹ is C(F), X² is C(H), and X³ is C(CF₃). In some embodiments is a compound of Formula (II″), (II′), or (II), or a pharmaceutically acceptable salt or solvate thereof, wherein X¹ is C(Cl), X² is C(H), and X³ is C(CF₃). In some embodiments is a compound of Formula (II″), (II′), or (II), or a pharmaceutically acceptable salt or solvate thereof, wherein X¹ is C(H), X² is C(H), and X³ is C(F). In some embodiments is a compound of Formula (II″), (II′), or (II), or a pharmaceutically acceptable salt or solvate thereof, wherein X¹ is C(H), X² is C(H), and X³ is C(Cl).

In some embodiments is a compound of Formula (II″), (II′), or (II), or a pharmaceutically acceptable salt or solvate thereof, wherein R² is selected from H, halogen, C₁₋₆alkyl, C₁₋₆haloalkyl, and —OR¹⁰. In some embodiments is a compound of Formula (II″), (II′), or (II), or a pharmaceutically acceptable salt or solvate thereof, wherein R² is H. In some embodiments is a compound of Formula (II″), (II′), or (II), or a pharmaceutically acceptable salt or solvate thereof, wherein R² is halogen. In some embodiments is a compound of Formula (II″), (II′), or (II), or a pharmaceutically acceptable salt or solvate thereof, wherein R² is F. In some embodiments is a compound of Formula (II″), (II′), or (II), or a pharmaceutically acceptable salt or solvate thereof, wherein R² is Cl. In some embodiments is a compound of Formula (II″), (II′), or (II), or a pharmaceutically acceptable salt or solvate thereof, wherein R² is C₁₋₆alkyl. In some embodiments is a compound of Formula (II″), (II′), or (II), or a pharmaceutically acceptable salt or solvate thereof, wherein R² is C₁₋₆haloalkyl. In some embodiments is a compound of Formula (II″), (II′), or (II), or a pharmaceutically acceptable salt or solvate thereof, wherein R² is —OR¹⁰. In some embodiments is a compound of Formula (II″), (II′), or (II), or a pharmaceutically acceptable salt or solvate thereof, wherein R² is —OH. In some embodiments is a compound of Formula (II″), (II′), or (II), or a pharmaceutically acceptable salt or solvate thereof, wherein R² is —OCH₃.

In some embodiments is a compound of Formula (II″), (II′), or (II), or a pharmaceutically acceptable salt or solvate thereof, wherein Z¹ and Z³ are CR⁵. In some embodiments is a compound of Formula (II″), (II′), or (II), or a pharmaceutically acceptable salt or solvate thereof, wherein Z¹ is N and Z³ is CR⁵. In some embodiments is a compound of Formula (II″), (II′), or (II), or a pharmaceutically acceptable salt or solvate thereof, wherein Z¹ is CR⁵ and Z³ is N. In some embodiments is a compound of Formula (II″), (II′), or (II), or a pharmaceutically acceptable salt or solvate thereof, wherein Z¹ and Z³ are N.

In some embodiments is a compound of Formula (II″), (II′), or (II), or a pharmaceutically acceptable salt or solvate thereof, wherein Z⁴ is CR⁸ and Z⁵ is CR⁵ or N. In some embodiments is a compound of Formula (II″), (II′), or (II), or a pharmaceutically acceptable salt or solvate thereof, wherein Z⁴ is CR⁸ and Z⁵ is CR⁵. In some embodiments is a compound of Formula (II″), (II′), or (II), or a pharmaceutically acceptable salt or solvate thereof, wherein Z⁴ is CR⁸ and Z⁵ is N. In some embodiments is a compound of Formula (II″), (II′), or (II), or a pharmaceutically acceptable salt or solvate thereof, wherein Z⁵ is CR⁸ and Z⁴ is CR⁵ or N. In some embodiments is a compound of Formula (II″), (II′), or (II), or a pharmaceutically acceptable salt or solvate thereof, wherein Z⁵ is CR⁸ and Z⁴ is CR⁵. In some embodiments is a compound of Formula (II″), (II′), or (II), or a pharmaceutically acceptable salt or solvate thereof, wherein Z⁵ is CR⁸ and Z⁴ is N.

In some embodiments is a compound of Formula (II″), (II′), or (II), or a pharmaceutically acceptable salt or solvate thereof, wherein each R⁵ is independently selected from H, halogen, C₁₋₆alkyl, and —OR¹⁰. In some embodiments is a compound of Formula (II″), (II′), or (II), or a pharmaceutically acceptable salt or solvate thereof, wherein each R⁵ is H. In some embodiments is a compound of Formula (II″), (II′), or (II), or a pharmaceutically acceptable salt or solvate thereof, wherein Z¹ and Z³ are C(H). In some embodiments is a compound of Formula (II″), (II′), or (II), or a pharmaceutically acceptable salt or solvate thereof, wherein Z¹ is N and Z³ are C(H). In some embodiments is a compound of Formula (II″), (II′), or (II), or a pharmaceutically acceptable salt or solvate thereof, wherein Z³ is N and Z¹ is C(H).

In some embodiments is a compound of Formula (II″), (II′), or (II), or a pharmaceutically acceptable salt or solvate thereof, wherein L¹ is a bond. In some embodiments is a compound of Formula (II″), (II′), or (II), or a pharmaceutically acceptable salt or solvate thereof, wherein L¹ is —O—. In some embodiments is a compound of Formula (II″), (II′), or (II), or a pharmaceutically acceptable salt or solvate thereof, wherein L¹ is —N(R¹⁰)—. In some embodiments is a compound of Formula (II″), (II′), or (II), or a pharmaceutically acceptable salt or solvate thereof, wherein L¹ is —N(H)—. In some embodiments is a compound of Formula (II″), (II′), or (II), or a pharmaceutically acceptable salt or solvate thereof, wherein L¹ is —C(R¹⁰)(R¹¹)N(R¹⁰)—. In some embodiments is a compound of Formula (II″), (II′), or (II), or a pharmaceutically acceptable salt or solvate thereof, wherein L¹ is —CH₂N(H)—. In some embodiments is a compound of Formula (II″), (II′), or (II), or a pharmaceutically acceptable salt or solvate thereof, wherein L¹ is —N(R¹⁰)C(R¹⁰)(R¹¹)—. In some embodiments is a compound of Formula (II″), (II′), or (II), or a pharmaceutically acceptable salt or solvate thereof, wherein L¹ is —N(H)CH₂—.

In some embodiments is a compound of Formula (II″), (II′), or (II), or a pharmaceutically acceptable salt or solvate thereof, wherein R¹ is selected from C₃₋₈ cycloalkyl and C₂₋₉heterocycloalkyl, wherein C₃₋₈cycloalkyl and C₂₋₉heterocycloalkyl are optionally substituted with one, two, or three R⁶. In some embodiments is a compound of Formula (II″), (II′), or (II), or a pharmaceutically acceptable salt or solvate thereof, wherein R¹ is C₂₋₉heterocycloalkyl optionally substituted with one, two, or three R⁶. In some embodiments is a compound of Formula (II″), (II′), or (II), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl, wherein piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl are optionally substituted with one, two, or three R⁶. In some embodiments is a compound of Formula (II″), (II′), or (II), or a pharmaceutically acceptable salt or solvate thereof, wherein R¹ is

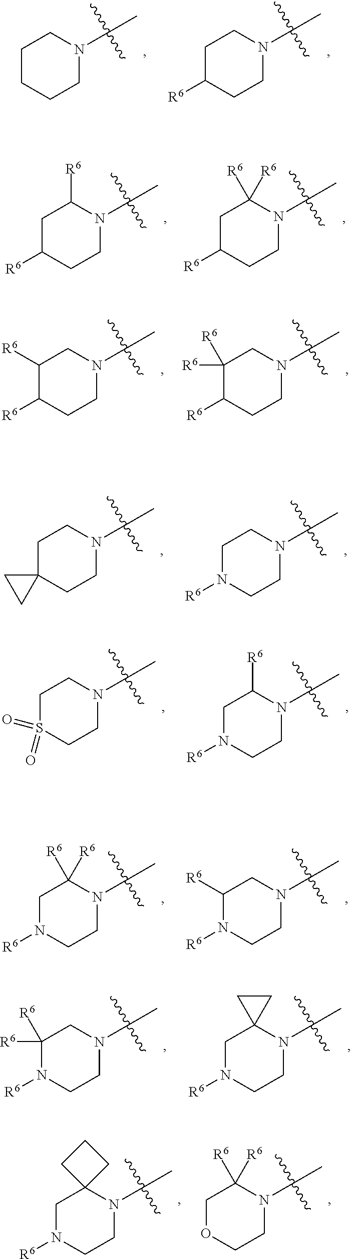

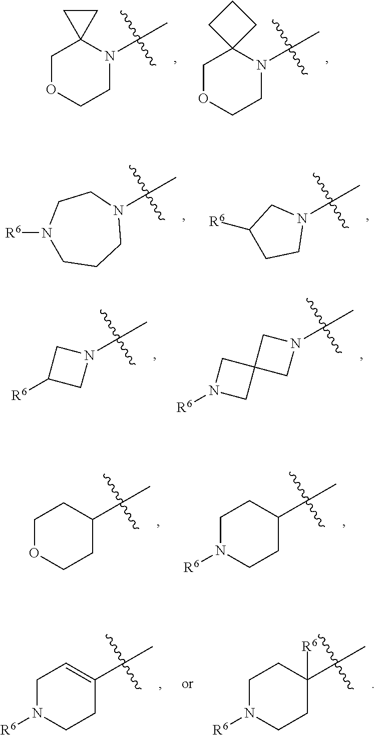

In some embodiments is a compound of Formula (II″), (II′), or (II), or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (II″), (II′), or (II), or a pharmaceutically acceptable salt or solvate thereof, wherein R¹ is

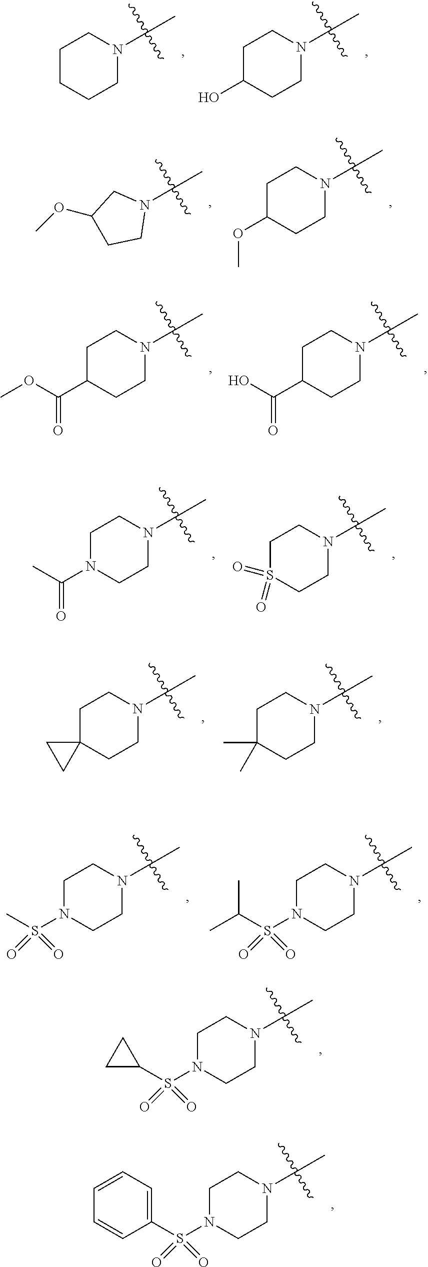

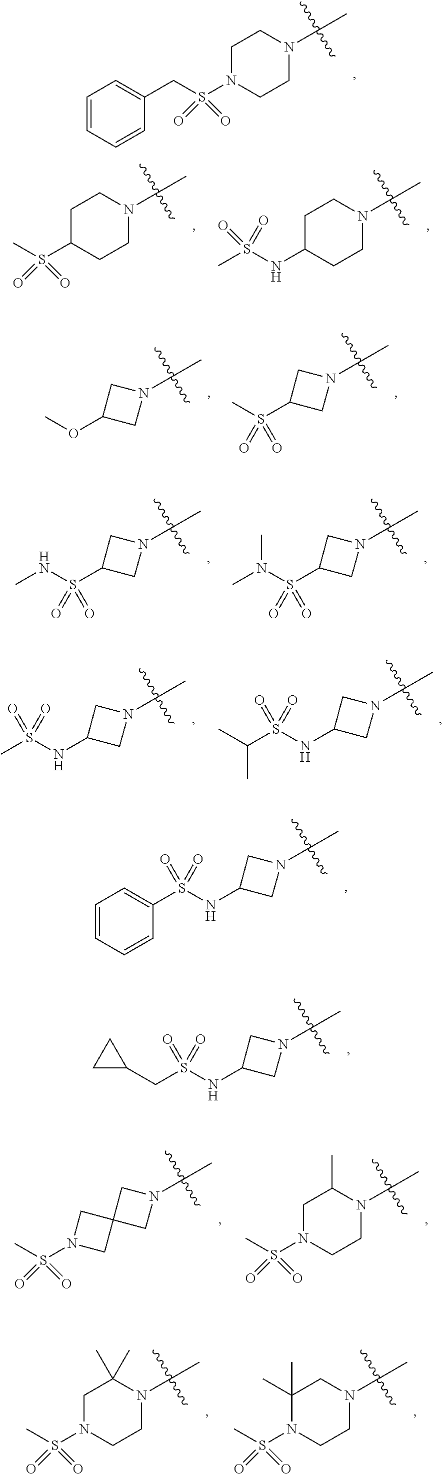

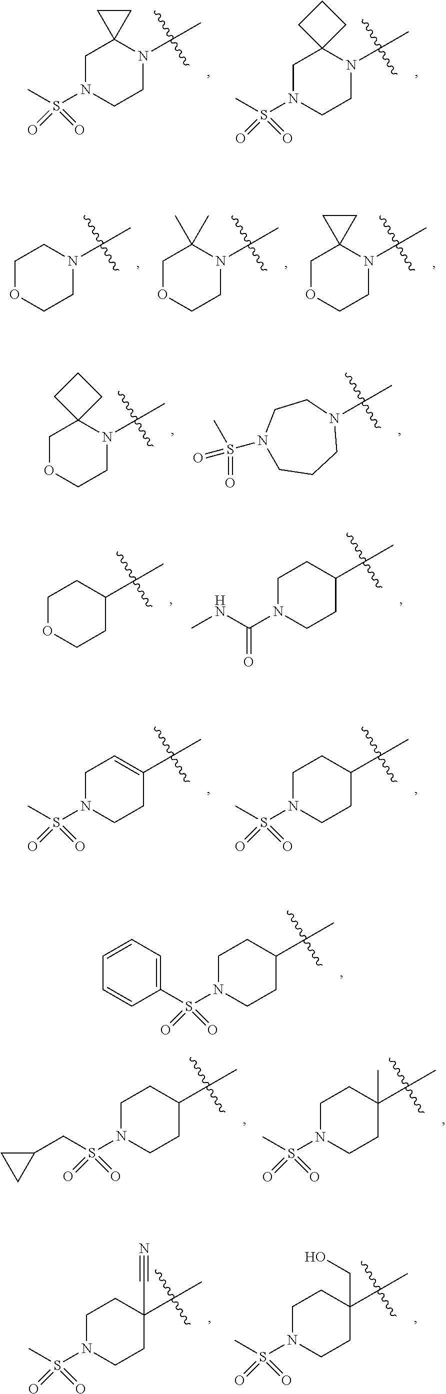

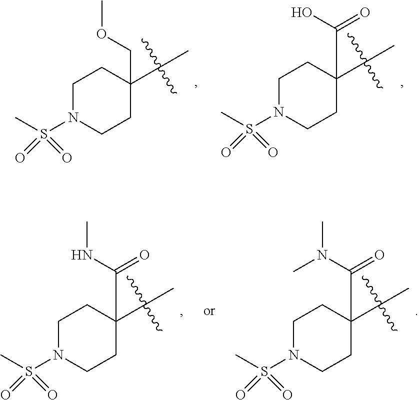 [SMILES: *C1(COC)CCN(S(C)(=O)=O)CC1 ; *C1(C(=O)O)CCN(S(C)(=O)=O)CC1 ; *C1(C(=O)NC)CCN(S(C)(=O)=O)CC1 ; *C1(C(=O)N(C)C)CCN(S(C)(=O)=O)CC1]

In some embodiments is a compound of Formula (II″), (II′), or (II), or a pharmaceutically acceptable salt or solvate thereof, wherein R¹ is

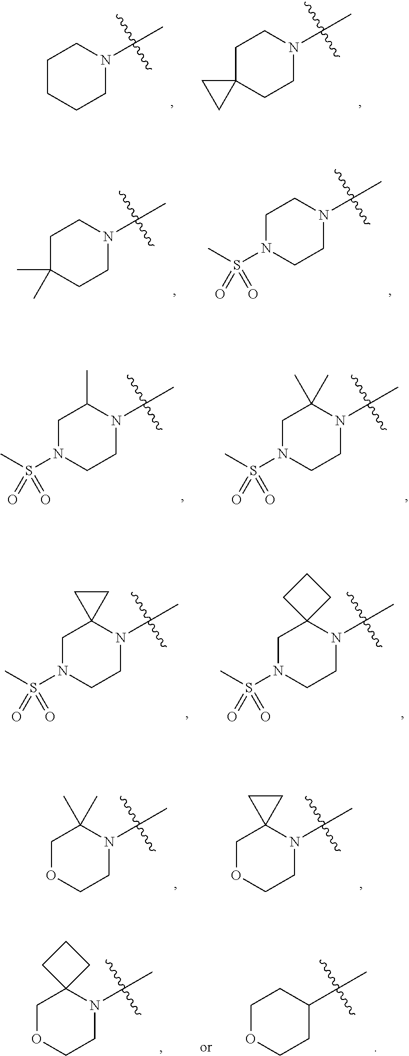

In some embodiments is a compound of Formula (II″), (II′), or (II), or a pharmaceutically acceptable salt or solvate thereof, wherein R¹ is

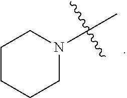 [SMILES: *N1CCCCC1]

In some embodiments is a compound of Formula (II″), (II′), or (II), or a pharmaceutically acceptable salt or solvate thereof, wherein R¹ is

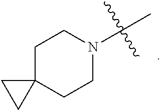 [SMILES: *N1CCC2(CC1)CC2]

In some embodiments is a compound of Formula (II″), (II′), or (II), or a pharmaceutically acceptable salt or solvate thereof, wherein R¹ is

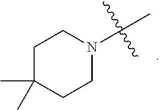 [SMILES: *N1CCC(C)(C)CC1]

In some embodiments is a compound of Formula (II″), (II′), or (II), or a pharmaceutically acceptable salt or solvate thereof, wherein R¹ is

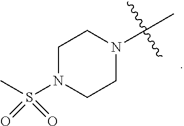 [SMILES: *N1CCN(S(C)(=O)=O)CC1]

In some embodiments is a compound of Formula (II″), (II′), or (II), or a pharmaceutically acceptable salt or solvate thereof, wherein R¹ is

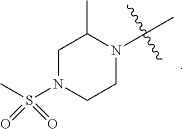 [SMILES: *N1CCN(S(C)(=O)=O)CC1C]

In some embodiments is a compound of Formula (II″), (II′), or (II), or a pharmaceutically acceptable salt or solvate thereof, wherein R¹ is

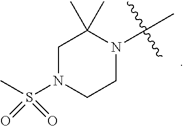 [SMILES: *N1CCN(S(C)(=O)=O)CC1(C)C]

In some embodiments is a compound of Formula (II″), (II′), or (II), or a pharmaceutically acceptable salt or solvate thereof, wherein R¹ is

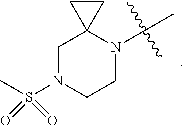 [SMILES: *N1CCN(S(C)(=O)=O)CC12CC2]

In some embodiments is a compound of Formula (II″), (II′), or (II), or a pharmaceutically acceptable salt or solvate thereof, wherein R¹ is

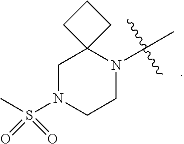 [SMILES: *N1CCN(S(C)(=O)=O)CC12CCC2]

In some embodiments is a compound of Formula (II″), (II′), or (II), or a pharmaceutically acceptable salt or solvate thereof, wherein R¹ is

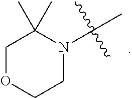 [SMILES: *N1CCOCC1(C)C]

In some embodiments is a compound of Formula (II″), (II′), or (II), or a pharmaceutically acceptable salt or solvate thereof, wherein R¹ is

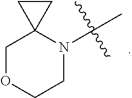 [SMILES: *N1CCOCC12CC2]

In some embodiments is a compound of Formula (II″), (II′), or (II), or a pharmaceutically acceptable salt or solvate thereof, wherein R¹ is

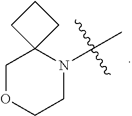 [SMILES: *N1CCOCC12CCC2]

In some embodiments is a compound of Formula (II″), (II′), or (II), or a pharmaceutically acceptable salt or solvate thereof, wherein R¹ is

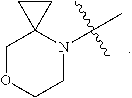 [SMILES: *N1CCOCC12CC2]

In some embodiments is a compound of Formula (II″), (II′), or (II), or a pharmaceutically acceptable salt or solvate thereof, wherein R¹ is

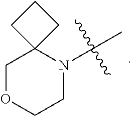 [SMILES: *N1CCOCC12CCC2]

In some embodiments is a compound of Formula (II″), (II′), or (II), or a pharmaceutically acceptable salt or solvate thereof, wherein R¹ is

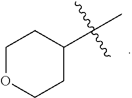 [SMILES: *C1CCOCC1]

In some embodiments is a compound of Formula (II″), (II′), or (II), or a pharmaceutically acceptable salt or solvate thereof, wherein R¹ is C₃₋₈cycloalkyl optionally substituted with one, two, or three R⁶.

In some embodiments is a compound of Formula (II″), (II′), or (II), or a pharmaceutically acceptable salt or solvate thereof, wherein R¹ is C₁₋₉heteroaryl substituted with one, two, or three R⁷. In some embodiments is a compound of Formula (II″), (II′), or (II), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷. In some embodiments is a compound of Formula (II″), (II′), or (II), or a pharmaceutically acceptable salt or solvate thereof, wherein R¹ is

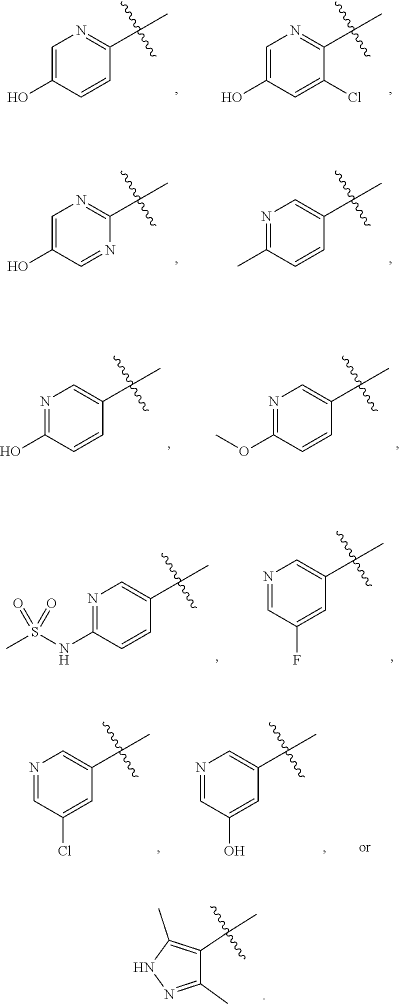

In some embodiments is a compound of Formula (IIa′), or a pharmaceutically acceptable salt or solvate thereof:

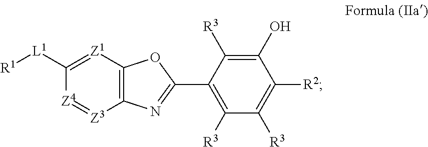

wherein:
Z¹ and Z³ are each independently CR⁵ or N;
Z⁴ is CR⁵ or N;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R³ and each R⁵ are each independently selected from H, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁷ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹⁴, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl; and
each R¹⁴ is independently selected C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆ cycloalkyl, C₂₋₉heterocycloalkyl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, and C₁₋₉heteroaryl.

In some embodiments is a compound of Formula (IIa′), or a pharmaceutically acceptable salt or solvate thereof, wherein each R³ is independently selected from H, halogen, C₁₋₆alkyl, C₁₋₆haloalkyl, and —OR¹⁰. In some embodiments is a compound of Formula (IIa′), or a pharmaceutically acceptable salt or solvate thereof, wherein each R³ is independently selected from H, halogen, C₁₋₆alkyl, and C₁₋₆haloalkyl. In some embodiments is a compound of Formula (IIa′), or a pharmaceutically acceptable salt or solvate thereof, wherein each R³ is independently selected from H, halogen, and C₁₋₆haloalkyl. In some embodiments is a compound of Formula (IIa′), or a pharmaceutically acceptable salt or solvate thereof, wherein each R³ is independently selected from H and C₁₋₆haloalkyl.

In some embodiments is a compound of Formula (IIa′), or a pharmaceutically acceptable salt or solvate thereof, wherein R² is selected from H, halogen, C₁₋₆alkyl, C₁₋₆ haloalkyl, and —OR¹⁰. In some embodiments is a compound of Formula (IIa′), or a pharmaceutically acceptable salt or solvate thereof, wherein R² is H. In some embodiments is a compound of Formula (IIa′), or a pharmaceutically acceptable salt or solvate thereof, wherein R² is halogen. In some embodiments is a compound of Formula (IIa′), or a pharmaceutically acceptable salt or solvate thereof, wherein R² is F. In some embodiments is a compound of Formula (IIa′), or a pharmaceutically acceptable salt or solvate thereof, wherein R² is Cl. In some embodiments is a compound of Formula (IIa′), or a pharmaceutically acceptable salt or solvate thereof, wherein R² is C₁₋₆alkyl. In some embodiments is a compound of Formula (IIa′), or a pharmaceutically acceptable salt or solvate thereof, wherein R² is C₁₋₆haloalkyl. In some embodiments is a compound of Formula (IIa′), or a pharmaceutically acceptable salt or solvate thereof, wherein R² is —OR¹⁰. In some embodiments is a compound of Formula (IIa′), or a pharmaceutically acceptable salt or solvate thereof, wherein R² is —OH. In some embodiments is a compound of Formula (IIa′), or a pharmaceutically acceptable salt or solvate thereof, wherein R² is —OCH₃.

In some embodiments is a compound of Formula (IIa′), or a pharmaceutically acceptable salt or solvate thereof, wherein Z¹ and Z³ are CR⁵. In some embodiments is a compound of Formula (IIa′), or a pharmaceutically acceptable salt or solvate thereof, wherein Z¹ is N and Z³ is CR⁵. In some embodiments is a compound of Formula (IIa′), or a pharmaceutically acceptable salt or solvate thereof, wherein Z¹ is CR⁵ and Z³ is N. In some embodiments is a compound of Formula (IIa′), or a pharmaceutically acceptable salt or solvate thereof, wherein Z¹ and Z³ are N.

In some embodiments is a compound of Formula (IIa′), or a pharmaceutically acceptable salt or solvate thereof, wherein Z⁴ is CR⁵. In some embodiments is a compound of Formula (IIa′), or a pharmaceutically acceptable salt or solvate thereof, wherein Z⁴ is N.

In some embodiments is a compound of Formula (IIa′), or a pharmaceutically acceptable salt or solvate thereof, wherein each R⁵ is independently selected from H, halogen, C₁₋₆alkyl, and —OR¹⁰. In some embodiments is a compound of Formula (IIa′), or a pharmaceutically acceptable salt or solvate thereof, wherein each R⁵ is H. In some embodiments is a compound of Formula (IIa′), or a pharmaceutically acceptable salt or solvate thereof, wherein Z¹, Z², and Z³ are C(H). In some embodiments is a compound of Formula (IIa′), or a pharmaceutically acceptable salt or solvate thereof, wherein Z¹ is N; and Z² and Z³ are C(H). In some embodiments is a compound of Formula (IIa′), or a pharmaceutically acceptable salt or solvate thereof, wherein Z² is N; and Z¹ and Z³ are C(H). In some embodiments is a compound of Formula (IIa′), or a pharmaceutically acceptable salt or solvate thereof, wherein Z³ is N; and Z¹ and Z² are C(H). In some embodiments is a compound of Formula (IIa′), or a pharmaceutically acceptable salt or solvate thereof, wherein Z¹ is C(H); and Z² and Z³ are N. In some embodiments is a compound of Formula (IIa′), or a pharmaceutically acceptable salt or solvate thereof, wherein Z² is C(H); and Z¹ and Z³ are N. In some embodiments is a compound of Formula (IIa′), or a pharmaceutically acceptable salt or solvate thereof, wherein Z³ is C(H); and Z¹ and Z² are N.

In some embodiments is a compound of Formula (IIa′), or a pharmaceutically acceptable salt or solvate thereof, wherein L¹ is a bond. In some embodiments is a compound of Formula (IIa′), or a pharmaceutically acceptable salt or solvate thereof, wherein L¹ is —O—. In some embodiments is a compound of Formula (IIa′), or a pharmaceutically acceptable salt or solvate thereof, wherein L¹ is —N(R¹⁰)—. In some embodiments is a compound of Formula (IIa′), or a pharmaceutically acceptable salt or solvate thereof, wherein L¹ is —N(H)—. In some embodiments is a compound of Formula (IIa′), or a pharmaceutically acceptable salt or solvate thereof, wherein L¹ is —C(R¹⁰)(R¹¹)N(R¹⁰)—. In some embodiments is a compound of Formula (IIa′), or a pharmaceutically acceptable salt or solvate thereof, wherein L¹ is —CH₂N(H)—. In some embodiments is a compound of Formula (IIa′), or a pharmaceutically acceptable salt or solvate thereof, wherein L¹ is —N(R¹⁰)C(R¹⁰)(R¹¹)—. In some embodiments is a compound of Formula (IIa′), or a pharmaceutically acceptable salt or solvate thereof, wherein L¹ is —N(H)CH₂—.

In some embodiments is a compound of Formula (IIa′), or a pharmaceutically acceptable salt or solvate thereof, wherein R¹ is selected from C₃₋₈cycloalkyl and C₂₋₉heterocycloalkyl, wherein C₃₋₈cycloalkyl and C₂₋₉heterocycloalkyl are optionally substituted with one, two, or three R⁶. In some embodiments is a compound of Formula (IIa′), or a pharmaceutically acceptable salt or solvate thereof, wherein R¹ is C₂₋₉heterocycloalkyl optionally substituted with one, two, or three R⁶. In some embodiments is a compound of Formula (IIa′), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl, wherein piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl are optionally substituted with one, two, or three R⁶. In some embodiments is a compound of Formula (IIa′), or a pharmaceutically acceptable salt or solvate thereof, wherein R¹ is

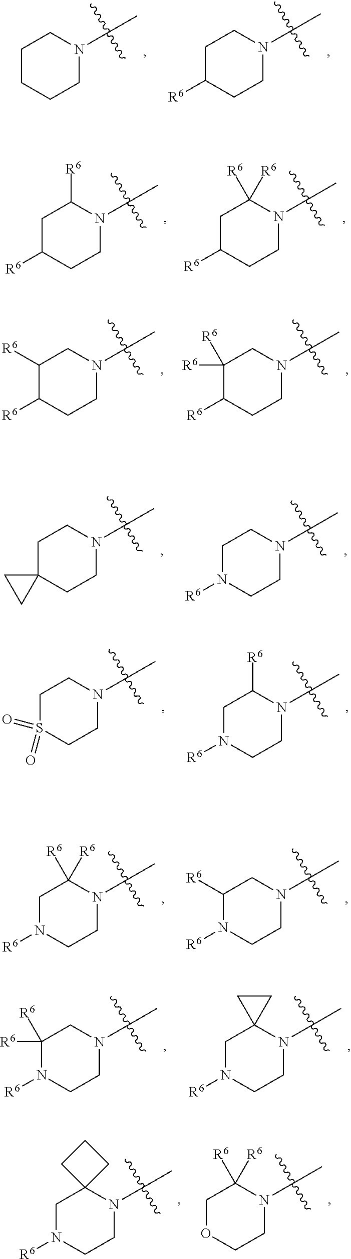

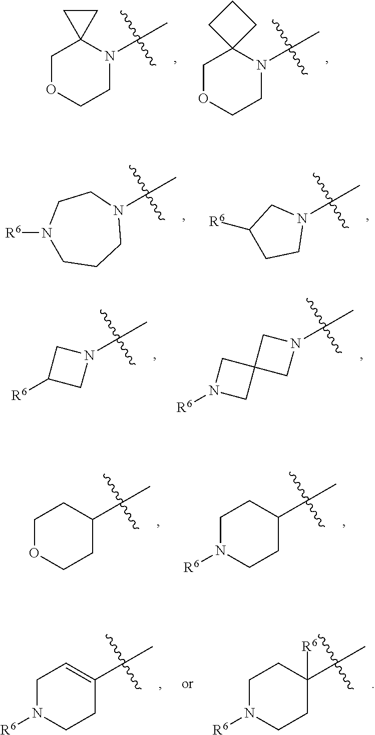

In some embodiments is a compound of Formula (IIa′), or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (IIa′), or a pharmaceutically acceptable salt or solvate thereof, wherein R¹ is

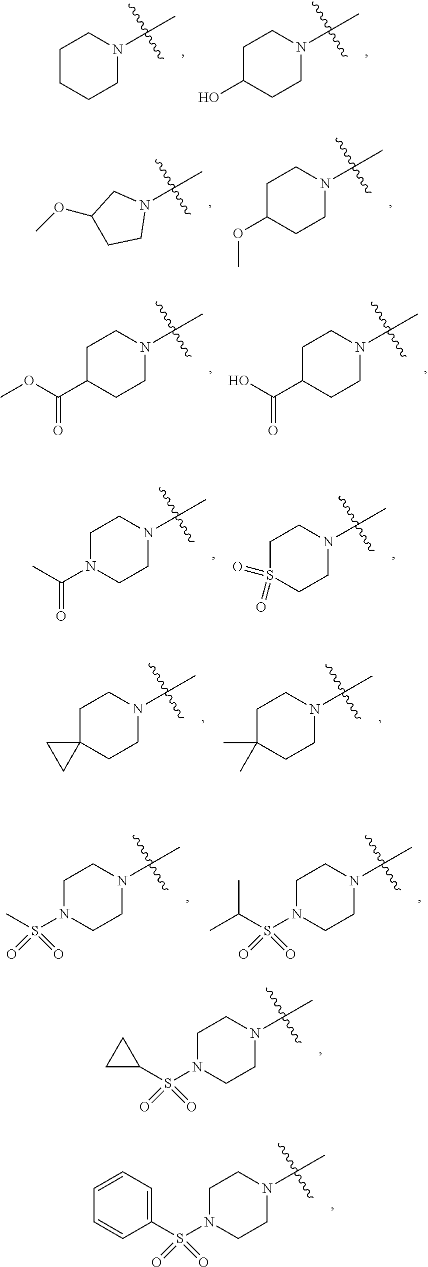

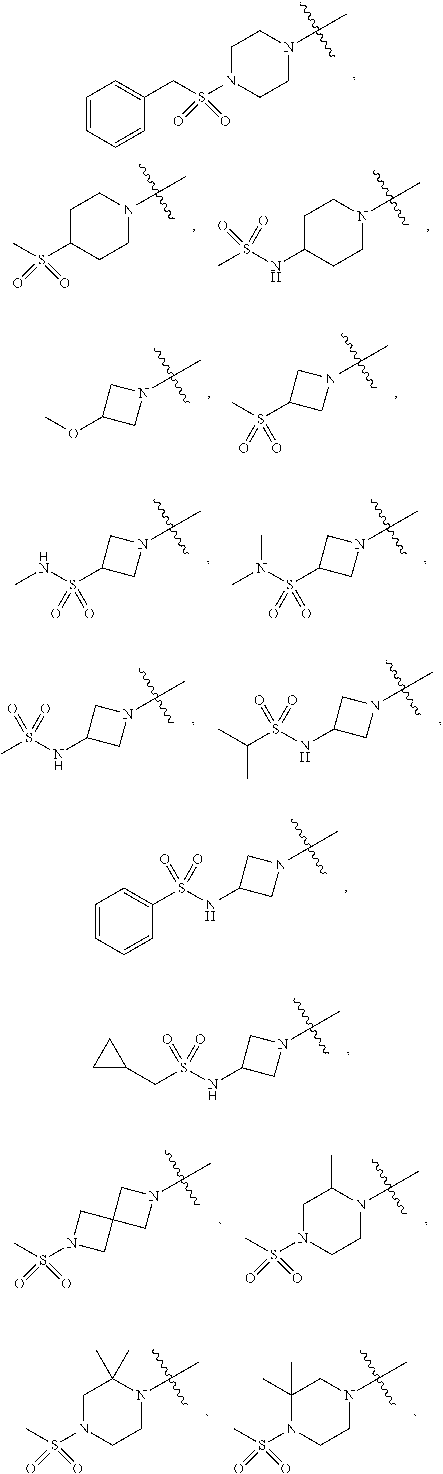

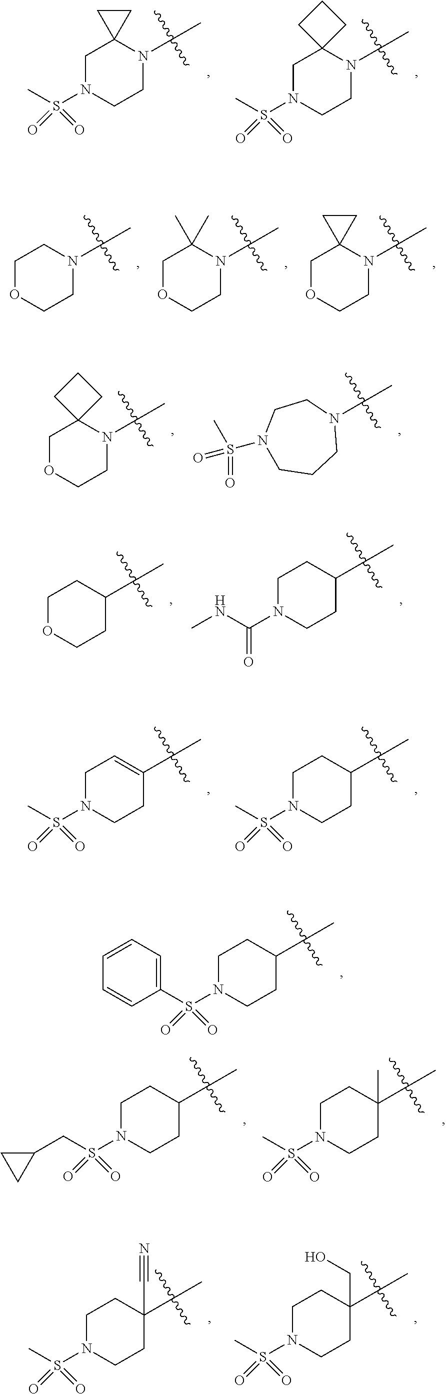

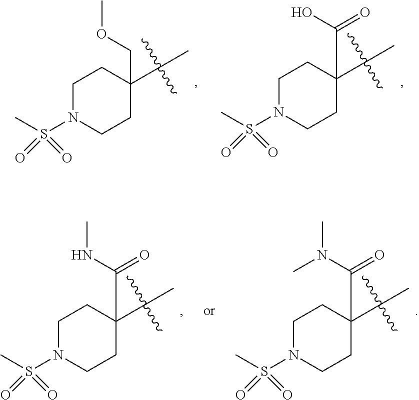 [SMILES: *C1(COC)CCN(S(C)(=O)=O)CC1 ; *C1(C(=O)O)CCN(S(C)(=O)=O)CC1 ; *C1(C(=O)NC)CCN(S(C)(=O)=O)CC1 ; *C1(C(=O)N(C)C)CCN(S(C)(=O)=O)CC1]

In some embodiments is a compound of Formula (IIa′), or a pharmaceutically acceptable salt or solvate thereof, wherein R¹ is

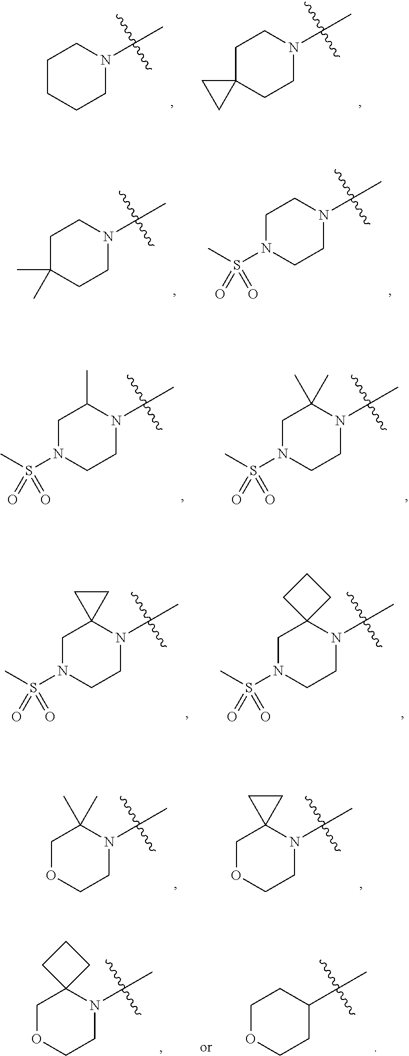

In some embodiments is a compound of Formula (IIa′), or a pharmaceutically acceptable salt or solvate thereof, wherein R¹ is

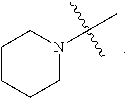 [SMILES: *N1CCCCC1]

In some embodiments is a compound of Formula (IIa′), or a pharmaceutically acceptable salt or solvate thereof, wherein R¹ is

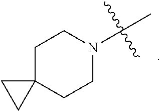 [SMILES: *N1CCC2(CC1)CC2]

In some embodiments is a compound of Formula (IIa′), or a pharmaceutically acceptable salt or solvate thereof, wherein R¹ is

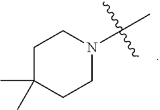 [SMILES: *N1CCC(C)(C)CC1]

In some embodiments is a compound of Formula (IIa′), or a pharmaceutically acceptable salt or solvate thereof, wherein R¹ is

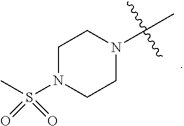 [SMILES: *N1CCN(S(C)(=O)=O)CC1]

In some embodiments is a compound of Formula (IIa′), or a pharmaceutically acceptable salt or solvate thereof, wherein R¹ is

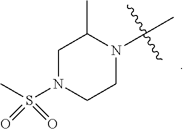 [SMILES: *N1CCN(S(C)(=O)=O)CC1C]

In some embodiments is a compound of Formula (IIa′), or a pharmaceutically acceptable salt or solvate thereof, wherein R¹ is

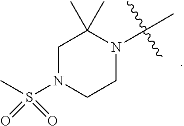 [SMILES: *N1CCN(S(C)(=O)=O)CC1(C)C]

In some embodiments is a compound of Formula (IIa′), or a pharmaceutically acceptable salt or solvate thereof, wherein R¹ is

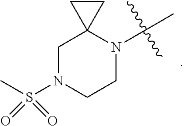 [SMILES: *N1CCN(S(C)(=O)=O)CC12CC2]

In some embodiments is a compound of Formula (IIa′), or a pharmaceutically acceptable salt or solvate thereof, wherein R¹ is

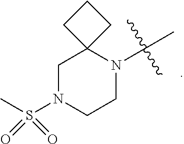 [SMILES: *N1CCN(S(C)(=O)=O)CC12CCC2]

In some embodiments is a compound of Formula (IIa′), or a pharmaceutically acceptable salt or solvate thereof, wherein R¹ is

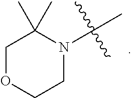 [SMILES: *N1CCOCC1(C)C]

In some embodiments is a compound of Formula (IIa′), or a pharmaceutically acceptable salt or solvate thereof, wherein R¹ is

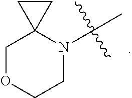 [SMILES: *N1CCOCC12CC2]

In some embodiments is a compound of Formula (IIa′), or a pharmaceutically acceptable salt or solvate thereof, wherein R¹ is

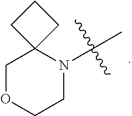 [SMILES: *N1CCOCC12CCC2]

In some embodiments is a compound of Formula (IIa′), or a pharmaceutically acceptable salt or solvate thereof, wherein R¹ is

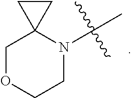 [SMILES: *N1CCOCC12CC2]

In some embodiments is a compound of Formula (IIa′), or a pharmaceutically acceptable salt or solvate thereof, wherein R¹ is

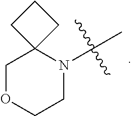 [SMILES: *N1CCOCC12CCC2]

In some embodiments is a compound of Formula (IIa′), or a pharmaceutically acceptable salt or solvate thereof, wherein R¹ is

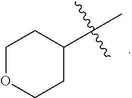 [SMILES: *C1CCOCC1]

In some embodiments is a compound of Formula (IIa′), or a pharmaceutically acceptable salt or solvate thereof, wherein R¹ is C₃₋₈cycloalkyl optionally substituted with one, two, or three R⁶.

In some embodiments is a compound of Formula (IIa′), or a pharmaceutically acceptable salt or solvate thereof, wherein R¹ is C₁₋₉heteroaryl substituted with one, two, or three R⁷. In some embodiments is a compound of Formula (IIa′), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷. In some embodiments is a compound of Formula (IIa′), or a pharmaceutically acceptable salt or solvate thereof, wherein R¹ is

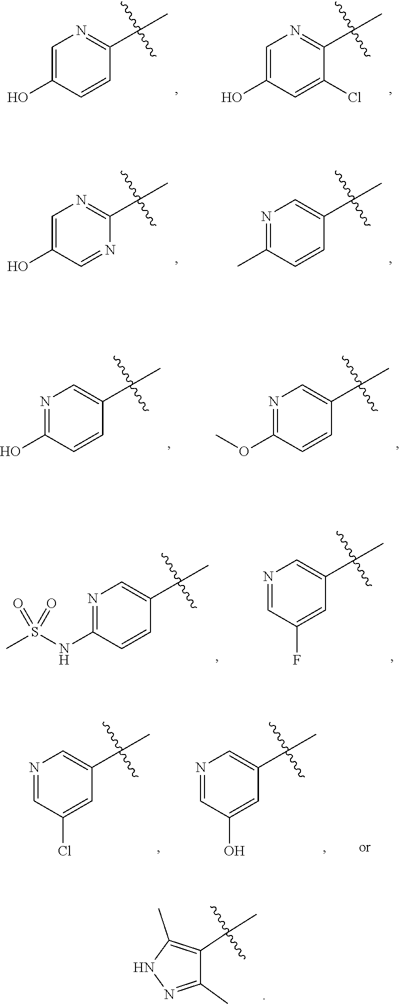

In some embodiments is a compound of Formula (IIb′), or a pharmaceutically acceptable salt or solvate thereof:

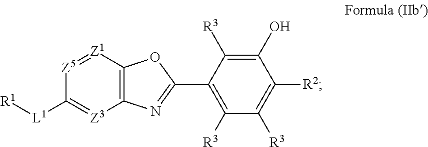

wherein:
Z¹ and Z³ are each independently CR⁵ or N;
Z⁵ is CR⁵ or N;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; and
   b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R³ and each R⁵ are each independently selected from H, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁷ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹⁴, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl; and
each R¹⁴ is independently selected C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆ cycloalkyl, C₂₋₉heterocycloalkyl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, and C₁₋₉heteroaryl.

In some embodiments is a compound of Formula (IIb′), or a pharmaceutically acceptable salt or solvate thereof, wherein each R³ is independently selected from H, halogen, C₁₋₆alkyl, C₁₋₆haloalkyl, and —OR¹⁰. In some embodiments is a compound of Formula (IIb′), or a pharmaceutically acceptable salt or solvate thereof, wherein each R³ is independently selected from H, halogen, C₁₋₆alkyl, and C₁₋₆haloalkyl. In some embodiments is a compound of Formula (IIb′), or a pharmaceutically acceptable salt or solvate thereof, wherein each R³ is independently selected from H, halogen, and C₁₋₆haloalkyl. In some embodiments is a compound of Formula (IIb′), or a pharmaceutically acceptable salt or solvate thereof, wherein each R³ is independently selected from H and C₁₋₆haloalkyl.

In some embodiments is a compound of Formula (IIb′), or a pharmaceutically acceptable salt or solvate thereof, wherein R² is selected from H, halogen, C₁₋₆alkyl, C₁₋₆ haloalkyl, and —OR¹⁰. In some embodiments is a compound of Formula (IIb′), or a pharmaceutically acceptable salt or solvate thereof, wherein R² is H. In some embodiments is a compound of Formula (IIb′), or a pharmaceutically acceptable salt or solvate thereof, wherein R² is halogen. In some embodiments is a compound of Formula (IIb′), or a pharmaceutically acceptable salt or solvate thereof, wherein R² is F. In some embodiments is a compound of Formula (IIb′), or a pharmaceutically acceptable salt or solvate thereof, wherein R² is Cl. In some embodiments is a compound of Formula (IIb′), or a pharmaceutically acceptable salt or solvate thereof, wherein R² is C₁₋₆alkyl. In some embodiments is a compound of Formula (IIb′), or a pharmaceutically acceptable salt or solvate thereof, wherein R² is C₁₋₆haloalkyl. In some embodiments is a compound of Formula (IIb′), or a pharmaceutically acceptable salt or solvate thereof, wherein R² is —OR¹⁰. In some embodiments is a compound of Formula (IIb′), or a pharmaceutically acceptable salt or solvate thereof, wherein R² is —OH. In some embodiments is a compound of Formula (IIb′), or a pharmaceutically acceptable salt or solvate thereof, wherein R² is —OCH₃.

In some embodiments is a compound of Formula (IIb′), or a pharmaceutically acceptable salt or solvate thereof, wherein Z¹ and Z³ are CR⁵. In some embodiments is a compound of Formula (IIb′), or a pharmaceutically acceptable salt or solvate thereof, wherein Z¹ is N and Z³ is CR⁵. In some embodiments is a compound of Formula (IIb′), or a pharmaceutically acceptable salt or solvate thereof, wherein Z¹ is CR⁵ and Z³ is N. In some embodiments is a compound of Formula (IIb′), or a pharmaceutically acceptable salt or solvate thereof, wherein Z¹ and Z³ are N.

In some embodiments is a compound of Formula (IIb′), or a pharmaceutically acceptable salt or solvate thereof, wherein Z⁵ is CR⁵. In some embodiments is a compound of Formula (IIb′), or a pharmaceutically acceptable salt or solvate thereof, wherein Z⁵ is N.

In some embodiments is a compound of Formula (IIb′), or a pharmaceutically acceptable salt or solvate thereof, wherein each R⁵ is independently selected from H, halogen, C₁₋₆alkyl, and —OR¹⁰. In some embodiments is a compound of Formula (IIb′), or a pharmaceutically acceptable salt or solvate thereof, wherein each R⁵ is H. In some embodiments is a compound of Formula (IIb′), or a pharmaceutically acceptable salt or solvate thereof, wherein Z¹, Z², and Z³ are C(H). In some embodiments is a compound of Formula (IIb′), or a pharmaceutically acceptable salt or solvate thereof, wherein Z¹ is N; and Z² and Z³ are C(H). In some embodiments is a compound of Formula (IIb′), or a pharmaceutically acceptable salt or solvate thereof, wherein Z² is N; and Z¹ and Z³ are C(H). In some embodiments is a compound of Formula (IIb′), or a pharmaceutically acceptable salt or solvate thereof, wherein Z³ is N; and Z¹ and Z² are C(H). In some embodiments is a compound of Formula (IIb′), or a pharmaceutically acceptable salt or solvate thereof, wherein Z¹ is C(H); and Z² and Z³ are N. In some embodiments is a compound of Formula (IIb′), or a pharmaceutically acceptable salt or solvate thereof, wherein Z² is C(H); and Z¹ and Z³ are N. In some embodiments is a compound of Formula (IIb′), or a pharmaceutically acceptable salt or solvate thereof, wherein Z³ is C(H); and Z¹ and Z² are N.

In some embodiments is a compound of Formula (IIb′), or a pharmaceutically acceptable salt or solvate thereof, wherein L¹ is a bond. In some embodiments is a compound of Formula (IIb′), or a pharmaceutically acceptable salt or solvate thereof, wherein L¹ is —O—. In some embodiments is a compound of Formula (IIb′), or a pharmaceutically acceptable salt or solvate thereof, wherein L¹ is —N(R¹⁰)—. In some embodiments is a compound of Formula (IIb′), or a pharmaceutically acceptable salt or solvate thereof, wherein L¹ is —N(H)—. In some embodiments is a compound of Formula (IIb′), or a pharmaceutically acceptable salt or solvate thereof, wherein L¹ is —C(R¹⁰)(R¹¹)N(R¹⁰)—. In some embodiments is a compound of Formula (IIb′), or a pharmaceutically acceptable salt or solvate thereof, wherein L¹ is —CH₂N(H)—. In some embodiments is a compound of Formula (IIb′), or a pharmaceutically acceptable salt or solvate thereof, wherein L¹ is —N(R¹⁰)C(R¹⁰)(R¹¹)—. In some embodiments is a compound of Formula (IIb′), or a pharmaceutically acceptable salt or solvate thereof, wherein L¹ is —N(H)CH₂—.

In some embodiments is a compound of Formula (IIb′), or a pharmaceutically acceptable salt or solvate thereof, wherein R¹ is selected from C₃₋₈cycloalkyl and C₂₋₉heterocycloalkyl, wherein C₃₋₈cycloalkyl and C₂₋₉heterocycloalkyl are optionally substituted with one, two, or three R⁶. In some embodiments is a compound of Formula (IIb′), or a pharmaceutically acceptable salt or solvate thereof, wherein R¹ is C₂₋₉heterocycloalkyl optionally substituted with one, two, or three R⁶. In some embodiments is a compound of Formula (IIb′), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, and aziridinyl, wherein piperidinyl, piperazinyl, morpholinyl, tetrahydropyranyl, tetrahydrofuranyl, pyrrolidinyl, oxetanyl, azetidinyl, aziridinyl, azepanyl, and diazepanyl are optionally substituted with one, two, or three R⁶. In some embodiments is a compound of Formula (IIb′), or a pharmaceutically acceptable salt or solvate thereof, wherein R¹ is

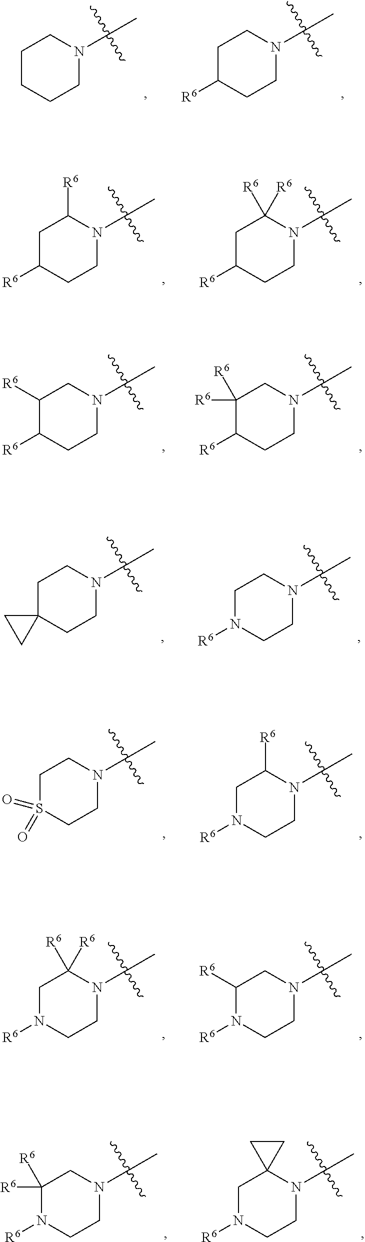

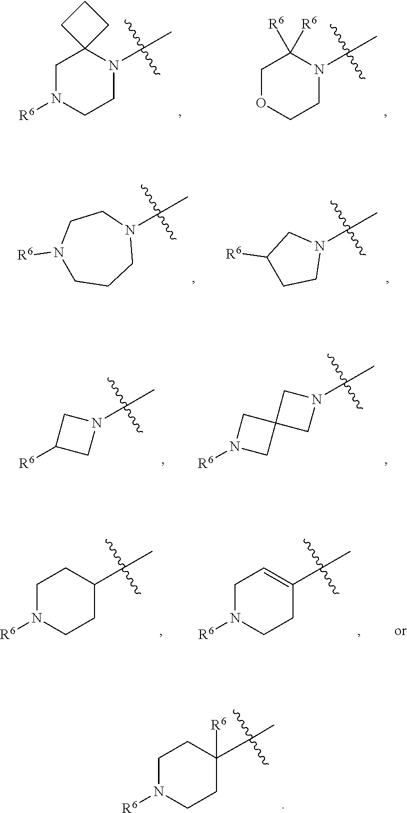

In some embodiments is a compound of Formula (IIb′), or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (IIb′) or a pharmaceutically acceptable salt or solvate thereof, wherein R¹ is

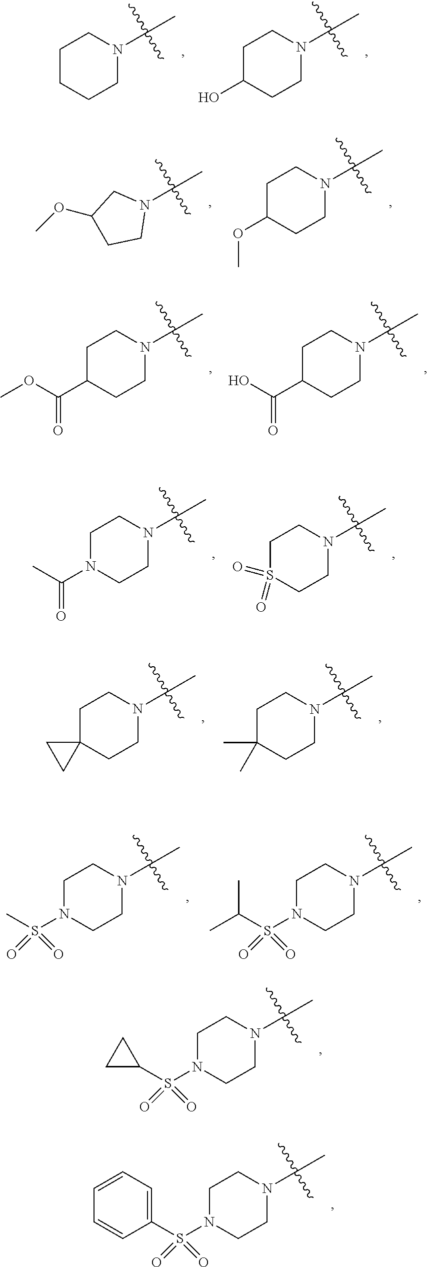

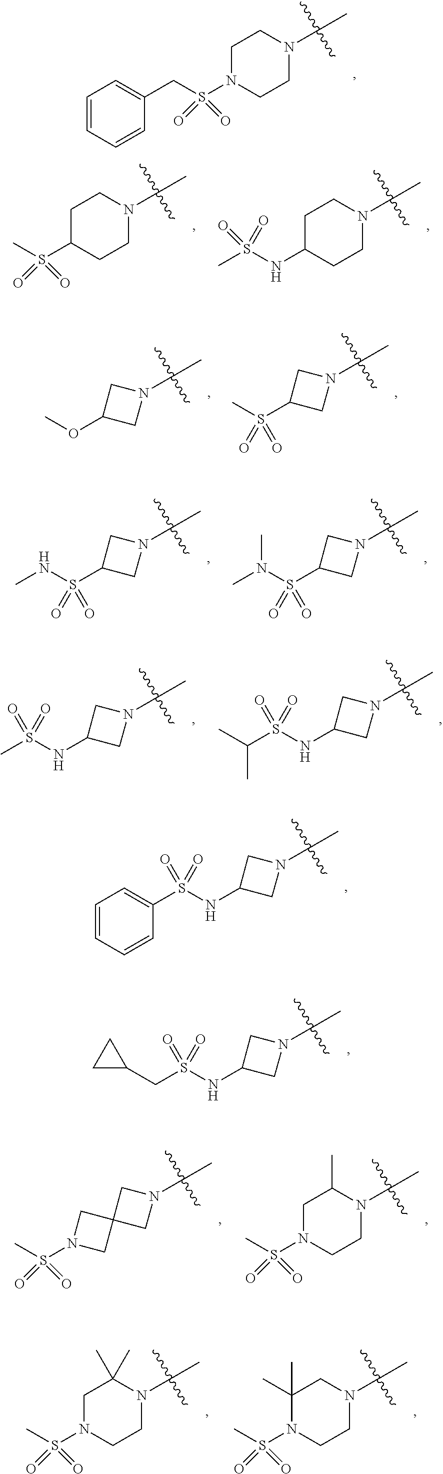

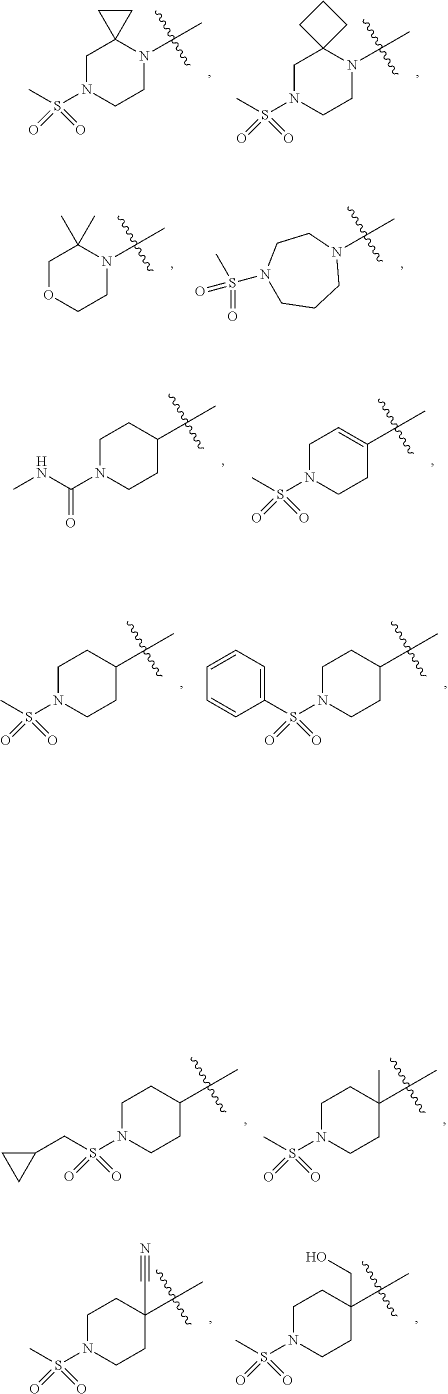

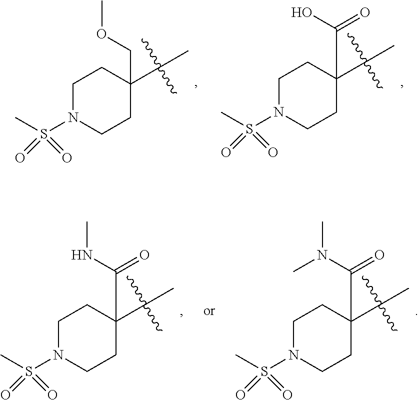 [SMILES: *C1(COC)CCN(S(C)(=O)=O)CC1 ; *C1(C(=O)O)CCN(S(C)(=O)=O)CC1 ; *C1(C(=O)NC)CCN(S(C)(=O)=O)CC1 ; *C1(C(=O)N(C)C)CCN(S(C)(=O)=O)CC1]

In some embodiments is a compound of Formula (IIb′), or a pharmaceutically acceptable salt or solvate thereof, wherein R¹ is

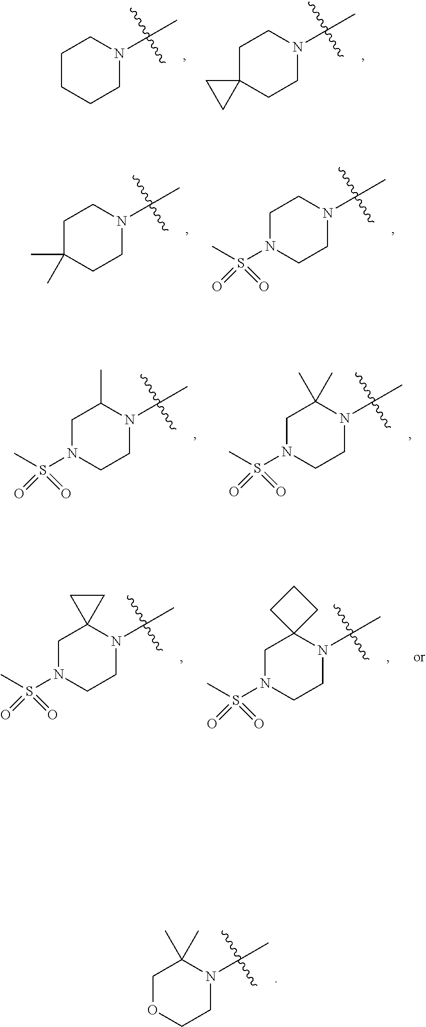

In some embodiments is a compound of Formula (IIb′), or a pharmaceutically acceptable salt or solvate thereof, wherein R¹ is

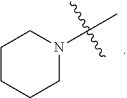 [SMILES: *N1CCCCC1]

In some embodiments is a compound of Formula (IIb′), or a pharmaceutically acceptable salt or solvate thereof, wherein R¹ is

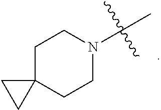 [SMILES: *N1CCC2(CC1)CC2]

In some embodiments is a compound of Formula (IIb′), or a pharmaceutically acceptable salt or solvate thereof, wherein R¹ is

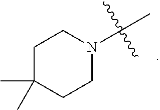 [SMILES: *N1CCC(C)(C)CC1]

In some embodiments is a compound of Formula (IIb′), or a pharmaceutically acceptable salt or solvate thereof, wherein R¹ is

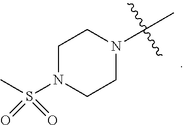 [SMILES: *N1CCN(S(C)(=O)=O)CC1]

In some embodiments is a compound of Formula (IIb′), or a pharmaceutically acceptable salt or solvate thereof, wherein R¹ is

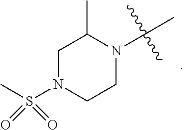 [SMILES: *N1CCN(S(C)(=O)=O)CC1C]

In some embodiments is a compound of Formula (IIb′), or a pharmaceutically acceptable salt or solvate thereof, wherein R¹ is

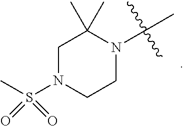 [SMILES: *N1CCN(S(C)(=O)=O)CC1(C)C]

In some embodiments is a compound of Formula (IIb′), or a pharmaceutically acceptable salt or solvate thereof, wherein R¹ is

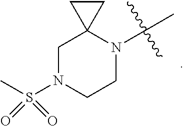 [SMILES: *N1CCN(S(C)(=O)=O)CC12CC2]

In some embodiments is a compound of Formula (IIb′), or a pharmaceutically acceptable salt or solvate thereof, wherein R¹ is

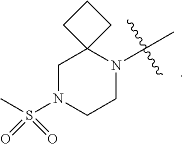 [SMILES: *N1CCN(S(C)(=O)=O)CC12CCC2]

In some embodiments is a compound of Formula (IIb′), or a pharmaceutically acceptable salt or solvate thereof, wherein R¹ is

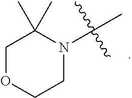 [SMILES: *N1CCOCC1(C)C]

In some embodiments is a compound of Formula (IIb′), or a pharmaceutically acceptable salt or solvate thereof, wherein R¹ is C₃₋₈cycloalkyl optionally substituted with one, two, or three R⁶.

In some embodiments is a compound of Formula (IIb′), or a pharmaceutically acceptable salt or solvate thereof, wherein R¹ is C₁₋₉heteroaryl substituted with one, two, or three R⁷. In some embodiments is a compound of Formula (IIb′), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷. In some embodiments is a compound of Formula (IIb′), or a pharmaceutically acceptable salt or solvate thereof, wherein R¹ is

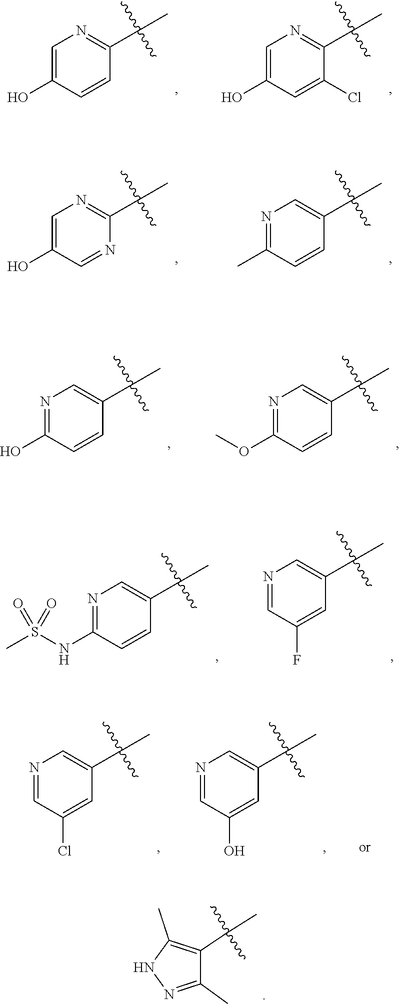

Any combination of the groups described above for the various variables is contemplated herein. Throughout the specification, groups and substituents thereof are chosen by one skilled in the field to provide stable moieties and compounds.

In some embodiments, compounds described herein include, but are not limited to, those described in Table 1.

TABLE 1

| Compound | Structure | Chemical Name |
|---|---|---|
| 1 | 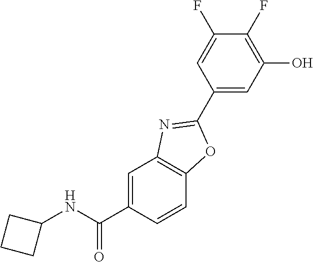 [SMILES: O=C(NC1CCC1)c1ccc2oc(-c3cc(O)c(F)c(F)c3)nc2c1] | N-Cyclobutyl-2-(3,4-difluoro-5- hydroxyphenyl)benzo[d]oxazole-5-carboxamide |
| 1.01 | 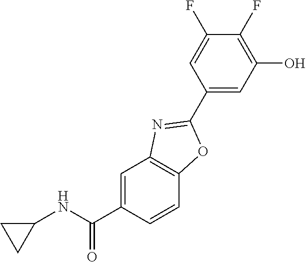 [SMILES: O=C(NC1CC1)c1ccc2oc(-c3cc(O)c(F)c(F)c3)nc2c1] | N-Cyclopropyl-2-(3,4-difluoro-5- hydroxyphenyl)benzo[d]oxazole-5-carboxamide |
| 1.02 | 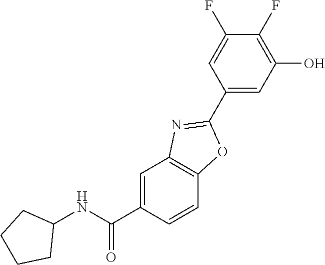 [SMILES: O=C(NC1CCCC1)c1ccc2oc(-c3cc(O)c(F)c(F)c3)nc2c1] | N-Cyclopentyl-2-(3,4-difluoro-5- hydroxyphenyl)benzo[d]oxazole-5-carboxamide |
| 1.03 | 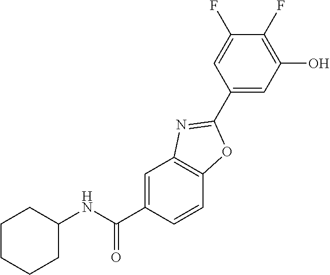 [SMILES: O=C(NC1CCCCC1)c1ccc2oc(-c3cc(O)c(F)c(F)c3)nc2c1] | N-Cyclohexyl-2-(3,4-difluoro-5- hydroxyphenyl)benzo[d]oxazole-5-carboxamide |
| 1.04 | 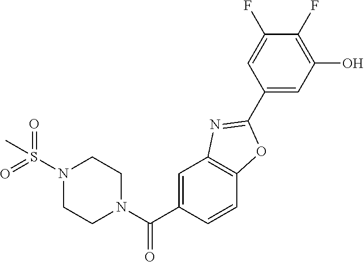 [SMILES: CS(=O)(=O)N1CCN(C(=O)c2ccc3oc(-c4cc(O)c(F)c(F)c4)nc3c2)CC1] | (2-(3,4-Difluoro-5- hydroxyphenyl)benzo[d]oxazol-5-yl)(4- (methylsulfonyl)piperazin-1-yl)methanone |
| 1.05 | 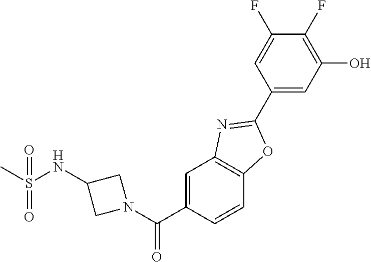 [SMILES: CS(=O)(=O)NC1CN(C(=O)c2ccc3oc(-c4cc(O)c(F)c(F)c4)nc3c2)C1] | N-(1-(2-(3,4-Difluoro-5- hydroxyphenyl)benzo[d]oxazole-5- carbonyl)azetidin-3-yl)methanesulfonamide |
| 1.06 | 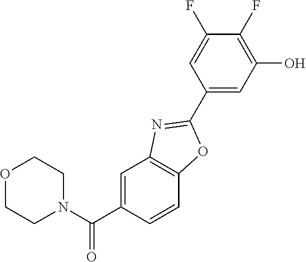 [SMILES: O=C(c1ccc2oc(-c3cc(O)c(F)c(F)c3)nc2c1)N1CCOCC1] | (2-(3,4-Difluoro-5- hydroxyphenyl)benzo[d]oxazol-5- yl)(morpholino)methanone |
| 1.07 | 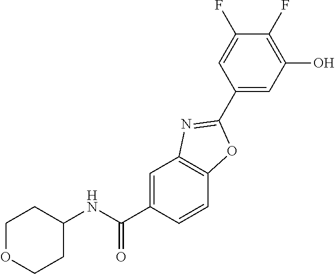 [SMILES: O=C(NC1CCOCC1)c1ccc2oc(-c3cc(O)c(F)c(F)c3)nc2c1] | 2-(3,4-Difluoro-5-hydroxyphenyl)-N- (tetrahydro-2H-pyran-4-yl)benzo[d]oxazole-5- carboxamide |
| 1.08 | 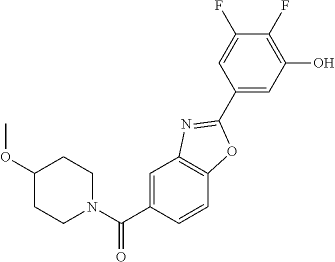 [SMILES: COC1CCN(C(=O)c2ccc3oc(-c4cc(O)c(F)c(F)c4)nc3c2)CC1] | (2-(3,4-Difluoro-5- hydroxyphenyl)benzo[d]oxazol-5-yl)(4- methoxypiperidin-1-yl)methanone |
| 1.09 | 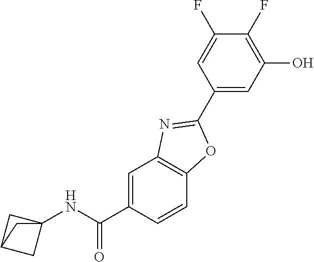 [SMILES: O=C(NC12CC(C1)C2)c1ccc2oc(-c3cc(O)c(F)c(F)c3)nc2c1] | N-(Bicyclo[1.1.1]pentan-1-yl)-2-(3,4-difluoro- 5-hydroxyphenyl)benzo[d]oxazole-5- carboxamide |
| 1.10 | 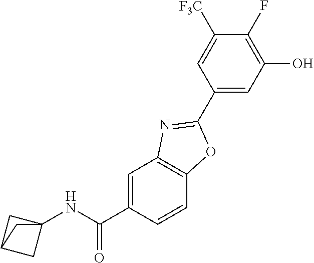 [SMILES: O=C(NC12CC(C1)C2)c1ccc2oc(-c3cc(O)c(F)c(C(F)(F)F)c3)nc2c1] | N-(Bicyclo[1.1.1]pentan-1-yl)-2-(4-fluoro-3- hydroxy-5- (trifluoromethyl)phenyl)benzo[d]oxazole-5- carboxamide |
| 1.11 | 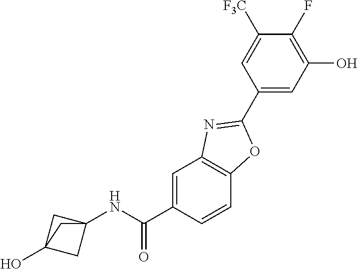 [SMILES: O=C(NC12CC(O)(C1)C2)c1ccc2oc(-c3cc(O)c(F)c(C(F)(F)F)c3)nc2c1] | 2-(4-Fluoro-3-hydroxy-5- (trifluoromethyl)phenyl)-N-(3- hydroxybicyclo[1.1.1]pentan-1- yl)benzo[d]oxazole-5-carboxamide |
| 1.12 | 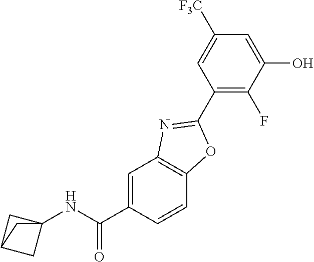 [SMILES: O=C(NC12CC(C1)C2)c1ccc2oc(-c3cc(C(F)(F)F)cc(O)c3F)nc2c1] | N-(Bicyclo[1.1.1]pentan-1-yl)-2-(2-fluoro-3- hydroxy-5- (trifluoromethyl)phenyl)benzo[d]oxazole-5- carboxamide |
| 1.13 | 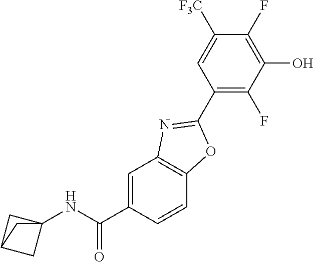 [SMILES: O=C(NC12CC(C1)C2)c1ccc2oc(-c3cc(C(F)(F)F)c(F)c(O)c3F)nc2c1] | N-(Bicyclo[1.1.1]pentan-1-yl)-2-(2,4-difluoro- 3-hydroxy-5- (trifluoromethyl)phenyl)benzo[d]oxazole-5- carboxamide |
| 1.14 | 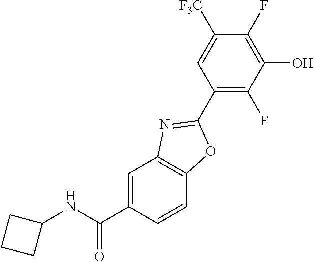 [SMILES: O=C(NC1CCC1)c1ccc2oc(-c3cc(C(F)(F)F)c(F)c(O)c3F)nc2c1] | N-Cyclobutyl-2-(2,4-difluoro-3-hydroxy-5- (trifluoromethyl)phenyl)benzo[d]oxazole-5- carboxamide |
| 1.15 | 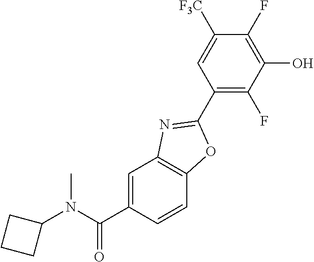 [SMILES: CN(C(=O)c1ccc2oc(-c3cc(C(F)(F)F)c(F)c(O)c3F)nc2c1)C1CCC1] | N-Cyclobutyl-2-(2,4-difluoro-3-hydroxy-5- (trifluoromethyl)phenyl)-N- methylbenzo[d]oxazole-5-carboxamide |
| 1.16 | 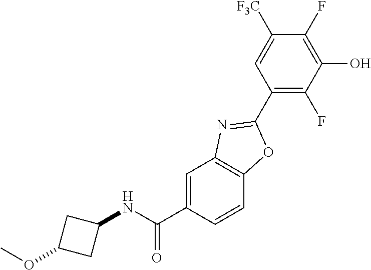 | 2-(2,4-Difluoro-3-hydroxy-5- (trifluoromethyl)phenyl)-N-(trans-3- methoxycyclobutyl)benzo[d]oxazole-5- carboxamide |
| 1.17 | 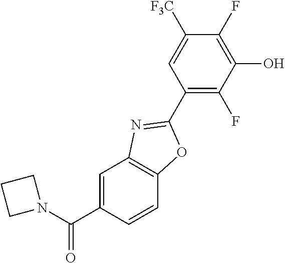 [SMILES: O=C(c1ccc2oc(-c3cc(C(F)(F)F)c(F)c(O)c3F)nc2c1)N1CCC1] | Azetidin-1-yl)(2-(2,4-difluoro-3-hydroxy-5- (trifluoromethyl)phenyl)benzo[d]oxazol-5- yl)methanone |
| 1.18 | 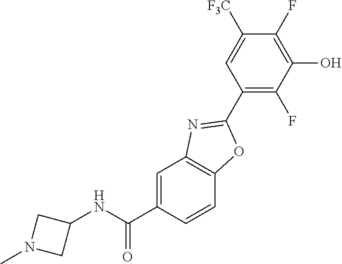 [SMILES: CN1CC(NC(=O)c2ccc3oc(-c4cc(C(F)(F)F)c(F)c(O)c4F)nc3c2)C1] | 2-(2,4-Difluoro-3-hydroxy-5- (trifluoromethyl)phenyl)-N-(1-methylazetidin-3- yl)benzo[d]oxazole-5-carboxamide |
| 1.19 | 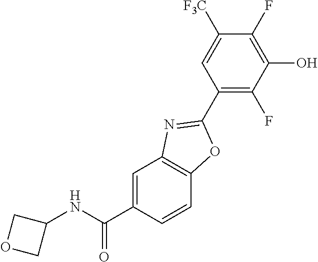 [SMILES: O=C(NC1COC1)c1ccc2oc(-c3cc(C(F)(F)F)c(F)c(O)c3F)nc2c1] | 2-(2,4-Difluoro-3-hydroxy-5- (trifluoromethyl)phenyl)-N-(oxetan-3- yl)benzo[d]oxazole-5-carboxamide |
| 1.20 | 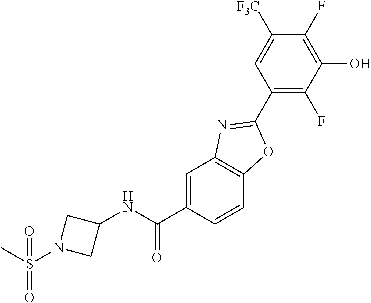 [SMILES: CS(=O)(=O)N1CC(NC(=O)c2ccc3oc(-c4cc(C(F)(F)F)c(F)c(O)c4F)nc3c2)C1] | 2-(2,4-Difluoro-3-hydroxy-5- (trifluoromethyl)phenyl)-N-(1- (methylsulfonyl)azetidin-3-yl)benzo[d]oxazole- 5-carboxamide |
| 1.21 | 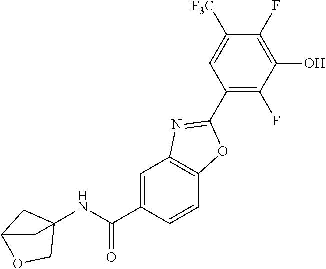 [SMILES: O=C(NC12COC(C1)C2)c1ccc2oc(-c3cc(C(F)(F)F)c(F)c(O)c3F)nc2c1] | N-(2-Oxabicyclo[2.1.1]hexan-4-yl)-2-(2,4- difluoro-3-hydroxy-5- (trifluoromethyl)phenyl)benzo[d]oxazole-5- carboxamide |
| 1.22 | 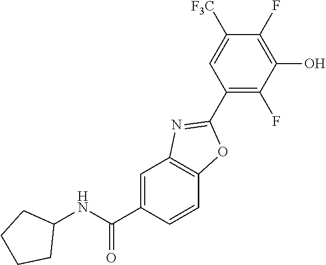 [SMILES: O=C(NC1CCCC1)c1ccc2oc(-c3cc(C(F)(F)F)c(F)c(O)c3F)nc2c1] | N-Cyclopentyl-2-(2,4-difluoro-3-hydroxy-5- (trifluoromethyl)phenyl)benzo[d]oxazole-5- carboxamide |
| 1.23 | 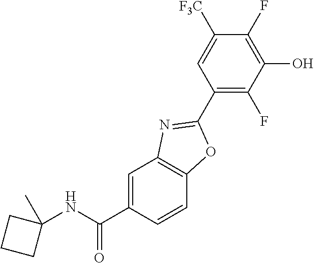 [SMILES: CC1(NC(=O)c2ccc3oc(-c4cc(C(F)(F)F)c(F)c(O)c4F)nc3c2)CCC1] | 2-(2,4-Difluoro-3-hydroxy-5- (trifluoromethyl)phenyl)-N-(1- methylcyclobutyl)benzo[d]oxazole-5- carboxamide |
| 1.24 | 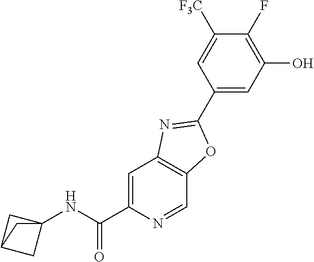 [SMILES: O=C(NC12CC(C1)C2)c1cc2nc(-c3cc(O)c(F)c(C(F)(F)F)c3)oc2cn1] | N-(Bicyclo[1.1.1]pentan-1-yl)-2-(4-fluoro-3- hydroxy-5-(trifluoromethyl)phenyl)oxazolo[5,4- c]pyridine-6-carboxamide |
| 1.25 | 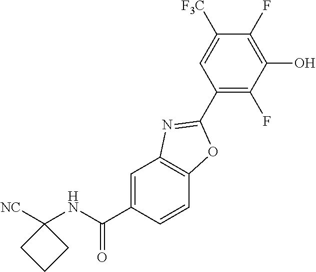 [SMILES: N#CC1(NC(=O)c2ccc3oc(-c4cc(C(F)(F)F)c(F)c(O)c4F)nc3c2)CCC1] | N-(1-Cyanocyclobutyl)-2-(2,4-difluoro-3- hydroxy-5- (trifluoromethyl)phenyl)benzo[d]oxazole-5- carboxamide |
| 1.26 | 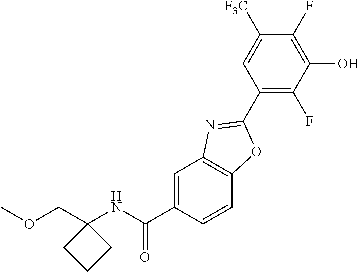 [SMILES: COCC1(NC(=O)c2ccc3oc(-c4cc(C(F)(F)F)c(F)c(O)c4F)nc3c2)CCC1] | 2-(2,4-Difluoro-3-hydroxy-5- (trifluoromethyl)phenyl)-N-(1- (methoxymethyl)cyclobutyl)benzo[d]oxazole-5- carboxamide |
| 1.27 | 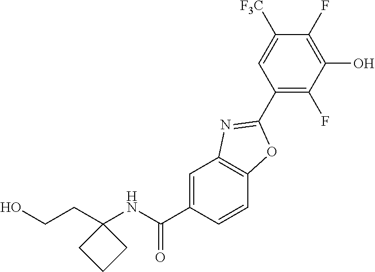 [SMILES: O=C(NC1(CCO)CCC1)c1ccc2oc(-c3cc(C(F)(F)F)c(F)c(O)c3F)nc2c1] | 2-(2,4-Difluoro-3-hydroxy-5- (trifluoromethyl)phenyl)-N-(1-(2- hydroxyethyl)cyclobutyl)benzo[d]oxazole-5- carboxamide |
| 1.28 | 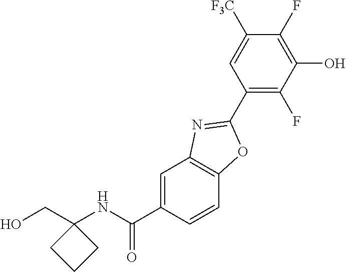 [SMILES: O=C(NC1(CO)CCC1)c1ccc2oc(-c3cc(C(F)(F)F)c(F)c(O)c3F)nc2c1] | 2-(2,4-Difluoro-3-hydroxy-5- (trifluoromethyl)phenyl)-N-(1- (hydroxymethyl)cyclobutyl)benzo[d]oxazole-5- carboxamide |
| 1.29 | 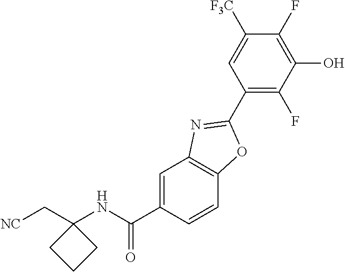 [SMILES: N#CCC1(NC(=O)c2ccc3oc(-c4cc(C(F)(F)F)c(F)c(O)c4F)nc3c2)CCC1] | N-(1-(Cyanomethyl)cyclobutyl)-2-(2,4-difluoro- 3-hydroxy-5- (trifluoromethyl)phenyl)benzo[d]oxazole-5- carboxamide |
| 1.30 | 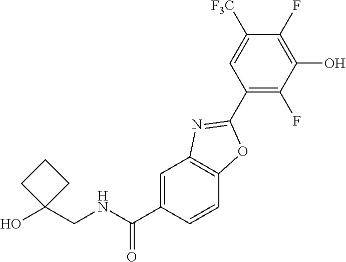 [SMILES: O=C(NCC1(O)CCC1)c1ccc2oc(-c3cc(C(F)(F)F)c(F)c(O)c3F)nc2c1] | 2-(2,4-Difluoro-3-hydroxy-5- (trifluoromethyl)phenyl)-N-((1- hydroxycyclobutyl)methyl)benzo[d]oxazole-5- carboxamide |
| 1.31 | 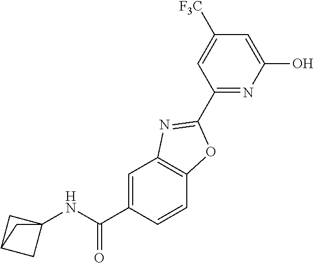 [SMILES: O=C(NC12CC(C1)C2)c1ccc2oc(-c3cc(C(F)(F)F)cc(O)n3)nc2c1] | N-(Bicyclo[1.1.1]pentan-1-yl)-2-(6-hydroxy-4- (trifluoromethyl)pyridin-2-yl)benzo[d]oxazole- 5-carboxamide |
| 2 | 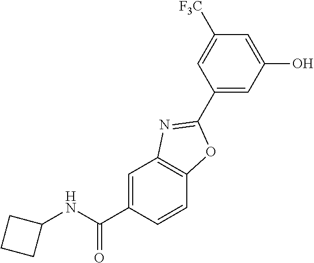 [SMILES: O=C(NC1CCC1)c1ccc2oc(-c3cc(O)cc(C(F)(F)F)c3)nc2c1] | N-Cyclobutyl-2-(3-hydroxy-5- (trifluoromethyl)phenyl)benzo[d]oxazole-5- carboxamide |
| 2.01 | 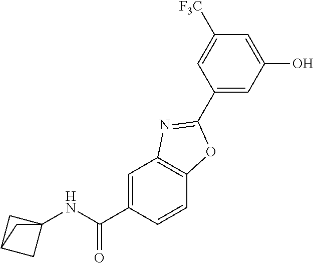 [SMILES: O=C(NC12CC(C1)C2)c1ccc2oc(-c3cc(O)cc(C(F)(F)F)c3)nc2c1] | N-(Bicyclo[1.1.1]pentan-1-yl)-2-(3-hydroxy-5- (trifluoromethyl)phenyl)benzo[d]oxazole-5- carboxamide |
| 2.02 | 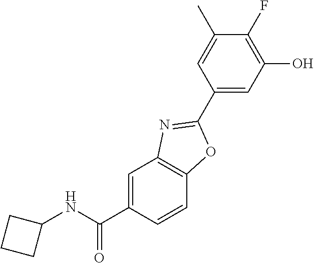 [SMILES: Cc1cc(-c2nc3cc(C(=O)NC4CCC4)ccc3o2)cc(O)c1F] | N-Cyclobutyl-2-(4-fluoro-3-hydroxy-5- methylphenyl)benzo[d]oxazole-5-carboxamide |
| 3 | 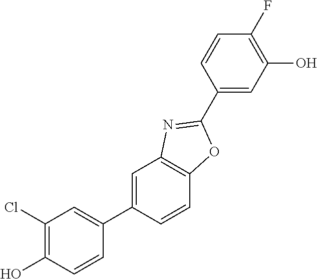 [SMILES: Oc1cc(-c2nc3cc(-c4ccc(O)c(Cl)c4)ccc3o2)ccc1F] | 2-Chloro-4-(2-(4-fluoro-3- hydroxyphenyl)benzo[d]oxazol-5-yl)phenol |
| 3.01 | 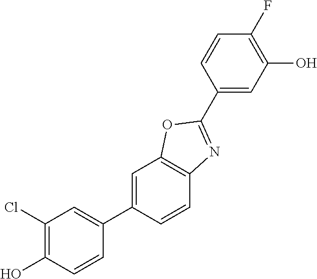 [SMILES: Oc1cc(-c2nc3ccc(-c4ccc(O)c(Cl)c4)cc3o2)ccc1F] | 2-Chloro-4-(2-(4-fluoro-3- hydroxyphenyl)benzo[d]oxazol-6-yl)phenol |
| 3.02 | 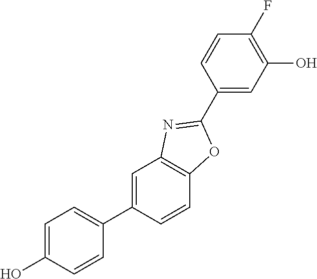 [SMILES: Oc1ccc(-c2ccc3oc(-c4ccc(F)c(O)c4)nc3c2)cc1] | 2-Fluoro-5-(5-(4- hydroxyphenyl)benzo[d]oxazol-2-yl)phenol |
| 3.03 | 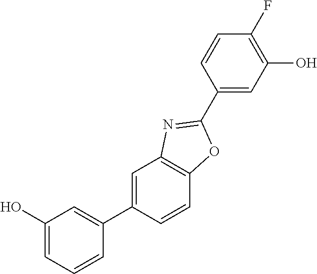 [SMILES: Oc1cccc(-c2ccc3oc(-c4ccc(F)c(O)c4)nc3c2)c1] | 2-Fluoro-5-(5-(3- hydroxyphenyl)benzo[d]oxazol-2-yl)phenol |
| 4 | 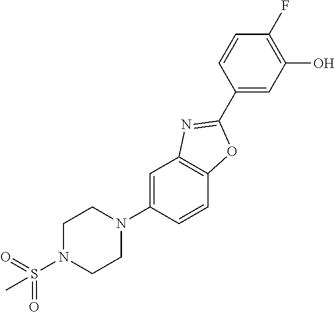 [SMILES: CS(=O)(=O)N1CCN(c2ccc3oc(-c4ccc(F)c(O)c4)nc3c2)CC1] | 2-Fluoro-5-(5-(4-(methylsulfonyl)piperazin-1- yl)benzo[d]oxazol-2-yl)phenol |
| 4.01 | 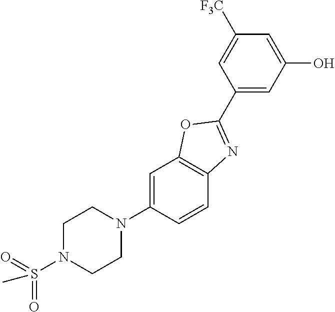 [SMILES: CS(=O)(=O)N1CCN(c2ccc3nc(-c4cc(O)cc(C(F)(F)F)c4)oc3c2)CC1] | 3-(6-(4-(Methylsulfonyl)piperazin-1- yl)benzo[d]oxazol-2-yl)-5- (trifluoromethyl)phenol |
| 4.02 | 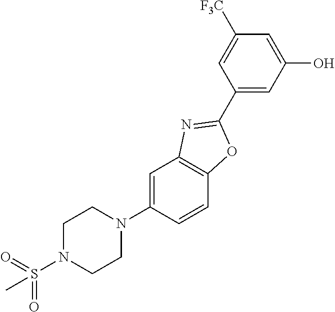 [SMILES: CS(=O)(=O)N1CCN(c2ccc3oc(-c4cc(O)cc(C(F)(F)F)c4)nc3c2)CC1] | 3-(5-(4-(Methylsulfonyl)piperazin-1- yl)benzo[d]oxazol-2-yl)-5- (trifluoromethyl)phenol |
| 5 | 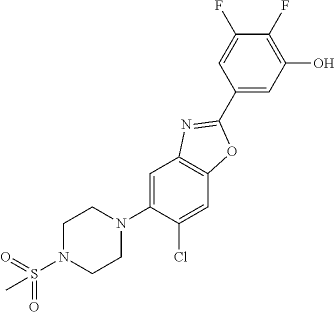 [SMILES: CS(=O)(=O)N1CCN(c2cc3nc(-c4cc(O)c(F)c(F)c4)oc3cc2Cl)CC1] | 5-(6-Chloro-5-(4-(methylsulfonyl)piperazin-1- yl)benzo[d]oxazol-2-yl)-2,3-difluorophenol |
| 5.01 | 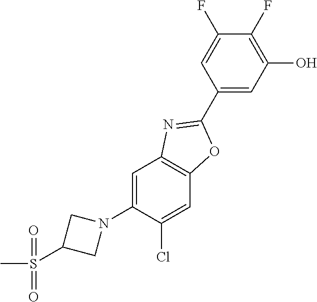 [SMILES: CS(=O)(=O)C1CN(c2cc3nc(-c4cc(O)c(F)c(F)c4)oc3cc2Cl)C1] | 5-(6-Chloro-5-(3-(methysulfonyl)azetidine-1- yl)benzo[d]oxazol-2-yl)-2,3-difluorophenol |
| 5.02 | 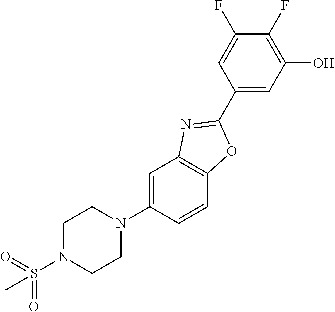 [SMILES: CS(=O)(=O)N1CCN(c2ccc3oc(-c4cc(O)c(F)c(F)c4)nc3c2)CC1] | 2,3-Difluoro-5-(5-(4-(methylsulfonyl)piperazin- 1-yl)benzo[d]oxazol-2-yl)phenol |
| 6 | 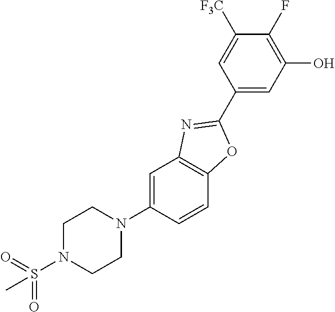 [SMILES: CS(=O)(=O)N1CCN(c2ccc3oc(-c4cc(O)c(F)c(C(F)(F)F)c4)nc3c2)CC1] | 2-Fluoro-5-(5-(4-(methylsulfonyl)piperazin-1- yl)benzo[d]oxazol-2-yl)-3- (trifluoromethyl)phenol |
| 6.01 | 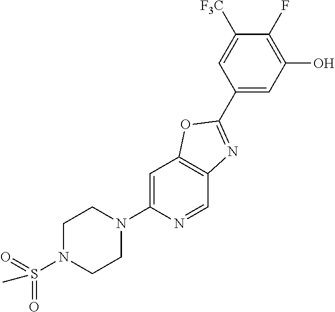 [SMILES: CS(=O)(=O)N1CCN(c2cc3oc(-c4cc(O)c(F)c(C(F)(F)F)c4)nc3cn2)CC1] | 3 2-Fluoro-5-(6-(4-(methylsulfonyl)piperazin-1- yl)oxazolo[4,5-c]pyridin-2-yl)-3- (trifluoromethyl)phenol |
| 6.02 | 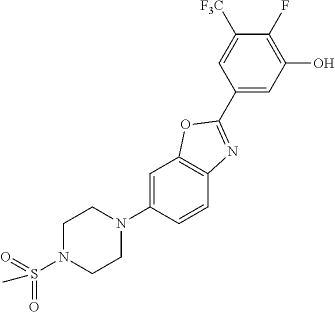 [SMILES: CS(=O)(=O)N1CCN(c2ccc3nc(-c4cc(O)c(F)c(C(F)(F)F)c4)oc3c2)CC1] | 2-Fluoro-5-(6-(4-(methylsulfonyl)piperazin-1- yl)benzo[d]oxazol-2-yl)-3- (trifluoromethyl)phenol |
| 7 | 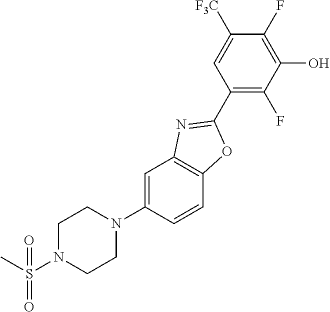 [SMILES: CS(=O)(=O)N1CCN(c2ccc3oc(-c4cc(C(F)(F)F)c(F)c(O)c4F)nc3c2)CC1] | 2,6-Difluoro-3-(5-(4-(methylsulfonyl)piperazin- 1-yl)benzo[d]oxazol-2-yl)-5- (trifluoromethyl)phenol |
| 7.01 | 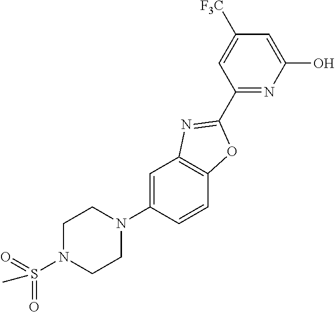 [SMILES: CS(=O)(=O)N1CCN(c2ccc3oc(-c4cc(C(F)(F)F)cc(O)n4)nc3c2)CC1] | 6-(5-(4-(Methylsulfonyl)piperazin-1- yl)benzo[d]oxazol-2-yl)-4- (trifluoromethyl)pyridin-2-ol |
| 7.02 | 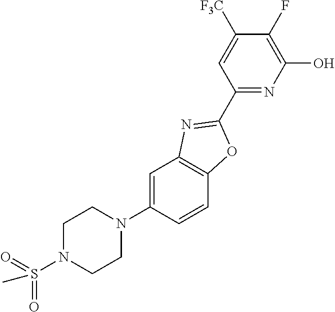 [SMILES: CS(=O)(=O)N1CCN(c2ccc3oc(-c4cc(C(F)(F)F)c(F)c(O)n4)nc3c2)CC1] | 3-Fluoro-6-(5-(4-(methylsulfonyl)piperazin-1- yl)benzo[d]oxazol-2-yl)-4- (trifluoromethyl)pyridin-2-ol |
| 8 | 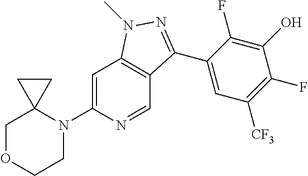 [SMILES: Cn1nc(-c2cc(C(F)(F)F)c(F)c(O)c2F)c2cnc(N3CCOCC34CC4)cc21] | 2,6-Difluoro-3-(1-methyl-6-(7-oxa-4- azaspiro[2.5]octan-4-yl)-1H-pyrazolo[4,3- c]pyridin-3-yl)-5-(trifluoromethyl)phenol |
| 9 | 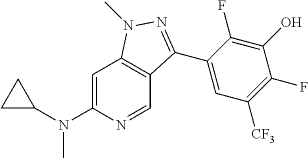 [SMILES: CN(c1cc2c(cn1)c(-c1cc(C(F)(F)F)c(F)c(O)c1F)nn2C)C1CC1] | 3-(6-(Cyclopropyl(methyl)amino)-1-methyl-1H- pyrazolo[4,3-c]pyridin-3-yl)-2,6-difluoro-5- (trifluoromethyl)phenol |
| 9.01 | 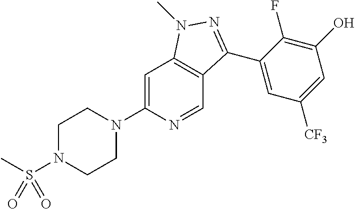 [SMILES: Cn1nc(-c2cc(C(F)(F)F)cc(O)c2F)c2cnc(N3CCN(S(C)(=O)=O)CC3)cc21] | 2-Fluoro-3-(1-methyl-6-(4- (methylsulfonyl)piperazin-1-yl)-1H- pyrazolo[4,3-c]pyridin-3-yl)-5- (trifluoromethyl)phenol |
| 9.02 | 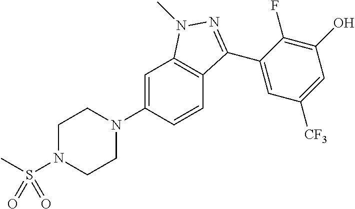 [SMILES: Cn1nc(-c2cc(C(F)(F)F)cc(O)c2F)c2ccc(N3CCN(S(C)(=O)=O)CC3)cc21] | 2-Fluoro-3-(1-methyl-6-(4- (methylsulfonyl)piperazin-1-yl)-1H-indazol-3- yl)-5-(trifluoromethyl)phenol |
| 9.03 | 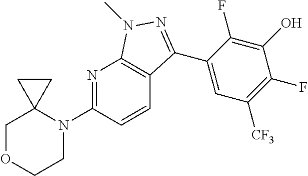 [SMILES: Cn1nc(-c2cc(C(F)(F)F)c(F)c(O)c2F)c2ccc(N3CCOCC34CC4)nc21] | 2,6-Difluoro-3-(1-methyl-6-(7-oxa-4- azaspiro[2.5]octan-4-yl)-1H-pyrazolo[3,4- b]pyridin-3-yl)-5-(trifluoromethyl)phenol |
| 9.04 | 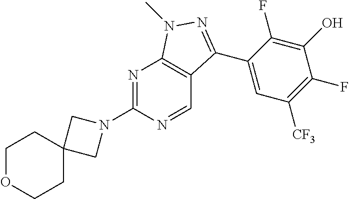 [SMILES: Cn1nc(-c2cc(C(F)(F)F)c(F)c(O)c2F)c2cnc(N3CC4(CCOCC4)C3)nc21] | 2,6-Difluoro-3-(1-methyl-6-(7-oxa-2- azaspiro[3.5]nonan-2-yl)-1H-pyrazolo[3,4- d]pyrimidin-3-yl)-5-(trifluoromethyl)phenol |
| 9.05 | 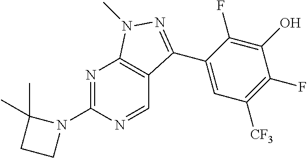 [SMILES: Cn1nc(-c2cc(C(F)(F)F)c(F)c(O)c2F)c2cnc(N3CCC3(C)C)nc21] | 3-(6-(2,2-Dimethylazetidin-1-yl)-1-methyl-1H- pyrazolo[3,4-d]pyrimidin-3-yl)-2,6-difluoro-5- (trifluoromethyl)phenol |
| 9.06 | 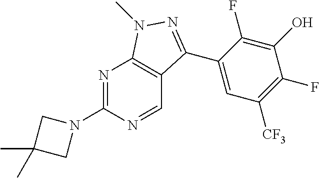 [SMILES: Cn1nc(-c2cc(C(F)(F)F)c(F)c(O)c2F)c2cnc(N3CC(C)(C)C3)nc21] | 3-(6-(3,3-Dimethylazetidin-1-yl)-1-methyl-1H- pyrazolo[3,4-d]pyrimidin-3-yl)-2,6-difluoro-5- (trifluoromethyl)phenol |
| 9.07 | 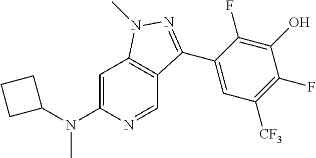 [SMILES: CN(c1cc2c(cn1)c(-c1cc(C(F)(F)F)c(F)c(O)c1F)nn2C)C1CCC1] | 3-(6-(Cyclobutyl(methyl)amino)-1-methyl-1H- pyrazolo[4,3-c]pyridin-3-yl)-2,6-difluoro-5- (trifluoromethyl)phenol |
| 9.08 | 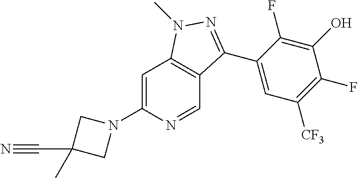 [SMILES: Cn1nc(-c2cc(C(F)(F)F)c(F)c(O)c2F)c2cnc(N3CC(C)(C#N)C3)cc21] | 1-(3-(2,4-Difluoro-3-hydroxy-5- (trifluoromethyl)phenyl)-1-methyl-1H- pyrazolo[4,3-c]pyridin-6-yl)-3-methylazetidine- 3-carbonitrile |
| 9.09 | 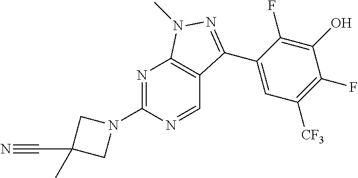 [SMILES: Cn1nc(-c2cc(C(F)(F)F)c(F)c(O)c2F)c2cnc(N3CC(C)(C#N)C3)nc21] | 1-(3-(2,4-Difluoro-3-hydroxy-5- (trifluoromethyl)phenyl)-1-methyl-1H- pyrazolo[3,4-d]pyrimidin-6-yl)-3- methylazetidine-3-carbonitrile |
| 9.10 | 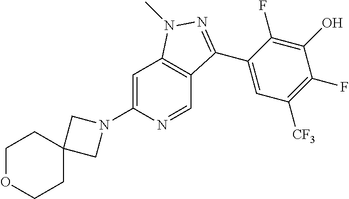 [SMILES: Cn1nc(-c2cc(C(F)(F)F)c(F)c(O)c2F)c2cnc(N3CC4(CCOCC4)C3)cc21] | 2,6-Difluoro-3-(1-methyl-6-(7-oxa-2- azaspiro[3.5]nonan-2-yl)-1H-pyrazolo[4,3- c]pyridin-3-yl)-5-(trifluoromethyl)phenol |
| 9.11 | 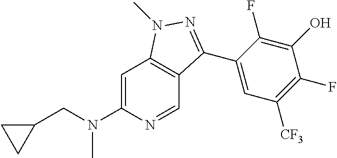 [SMILES: CN(CC1CC1)c1cc2c(cn1)c(-c1cc(C(F)(F)F)c(F)c(O)c1F)nn2C] | 3-(6-((Cyclopropylmethyl)(methyl)amino)-1- methyl-1H-pyrazolo[4,3-c]pyridin-3-yl)-2,6- difluoro-5-(trifluoromethyl)phenol |
| 9.12 | 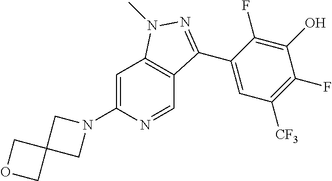 [SMILES: Cn1nc(-c2cc(C(F)(F)F)c(F)c(O)c2F)c2cnc(N3CC4(COC4)C3)cc21] | 2,6-Difluoro-3-(1-methyl-6-(2-oxa-6- azaspiro[3.3]heptan-6-yl)-1H-pyrazolo[4,3- c]pyridin-3-yl)-5-(trifluoromethyl)phenol |
| 9.13 | 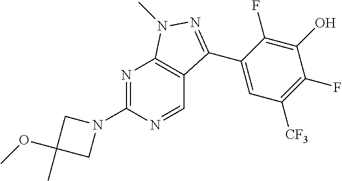 [SMILES: COC1(C)CN(c2ncc3c(-c4cc(C(F)(F)F)c(F)c(O)c4F)nn(C)c3n2)C1] | 2,6-Difluoro-3-(6-(3-methoxy-3- methylazetidin-1-yl)-1-methyl-1H- pyrazolo[3,4-d]pyrimidin-3-yl)-5- (trifluoromethyl)phenol |
| 9.14 | 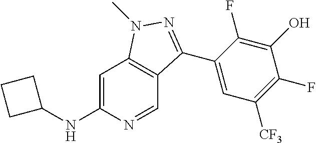 [SMILES: Cn1nc(-c2cc(C(F)(F)F)c(F)c(O)c2F)c2cnc(NC3CCC3)cc21] | 3-(6-(Cyclobutylamino)-1-methyl-1H- pyrazolo[4,3-c]pyridin-3-yl)-2,6-difluoro-5- (trifluoromethyl)phenol |
| 9.15 | 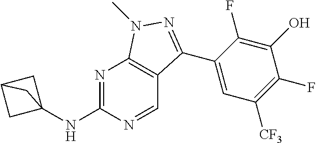 [SMILES: Cn1nc(-c2cc(C(F)(F)F)c(F)c(O)c2F)c2cnc(NC34CC(C3)C4)nc21] | 3-(6-(Bicyclo[1.1.1]pentan-1-ylamino)-1- methyl-1H-pyrazolo[3,4-d]pyrimidin-3-yl)-2,6- difluoro-5-(trifluoromethyl)phenol |
| 9.16 | 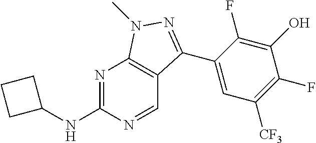 [SMILES: Cn1nc(-c2cc(C(F)(F)F)c(F)c(O)c2F)c2cnc(NC3CCC3)nc21] | 3-(6-(Cyclobutylamino)-1-methyl-1H- pyrazolo[3,4-d]pyrimidin-3-yl)-2,6-difluoro-5- (trifluoromethyl)phenol |
| 9.17 | 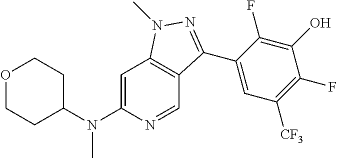 [SMILES: CN(c1cc2c(cn1)c(-c1cc(C(F)(F)F)c(F)c(O)c1F)nn2C)C1CCOCC1] | 2,6-Difluoro-3-(1-methyl-6-(methyl(tetrahydro- 2H-pyran-4-yl)amino)-1H-pyrazolo[4,3- c]pyridin-3-yl)-5-(trifluoromethyl)phenol |
| 9.18 | 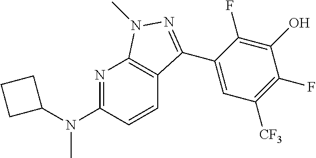 [SMILES: CN(c1ccc2c(-c3cc(C(F)(F)F)c(F)c(O)c3F)nn(C)c2n1)C1CCC1] | 3-(6-(Cyclobutyl(methyl)amino)-1-methyl-1H- pyrazolo[3,4-b]pyridin-3-yl)-2,6-difluoro-5- (trifluroomethyl)phenol |
| 9.19 | 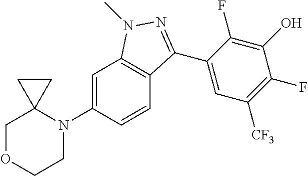 [SMILES: Cn1nc(-c2cc(C(F)(F)F)c(F)c(O)c2F)c2ccc(N3CCOCC34CC4)cc21] | 2,6-Difluoro-3-(1-methyl-6-(7-oxa-4- azaspiro[2.5]octan-4-yl)-1H-indazol-3-yl)-5- (trifluoromethyl)phenol |
| 9.20 | 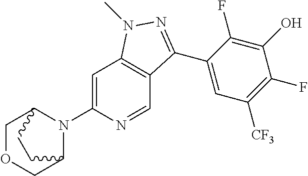 [SMILES: Cn1nc(-c2cc(C(F)(F)F)c(F)c(O)c2F)c2cnc(N3C4CCC3COC4)cc21] | 3-(6-(3-Oxa-8-azabicyclo[3.2.1]octan-8-yl)-1- methyl-1H-pyrazolo[4,3-c]pyridin-3-yl)-2,6- difluoro-5-(trifluoromethyl)phenol |
| 9.21 | 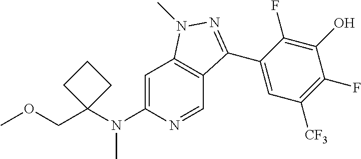 [SMILES: COCC1(N(C)c2cc3c(cn2)c(-c2cc(C(F)(F)F)c(F)c(O)c2F)nn3C)CCC1] | 2,6-Difluoro-3-(6-((1- (methoxymethyl)cyclobutyl)(methyl)amino)-1- methyl-1H-pyrazolo[4,3-c]pyridin-3-yl)-5- (trifluoromethyl)phenol |
| 9.22 | 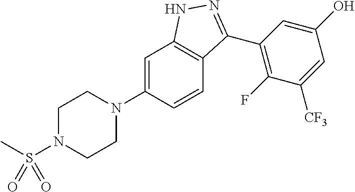 [SMILES: CS(=O)(=O)N1CCN(c2ccc3c(-c4cc(O)cc(C(F)(F)F)c4F)n[nH]c3c2)CC1] | 4-Fluoro-3-(6-(4-(methylsulfonyl)piperazin-1- yl)-1H-indazol-3-yl)-5-(trifluoromethyl)phenol |
| 9.23 | 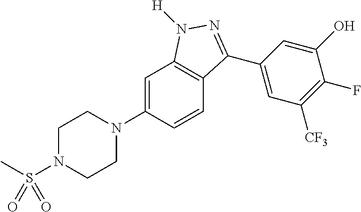 [SMILES: CS(=O)(=O)N1CCN(c2ccc3c(-c4cc(O)c(F)c(C(F)(F)F)c4)n[nH]c3c2)CC1] | 2-Fluoro-5-(6-(4-(methylsulfonyl)piperazin-1- yl)-1H-indazol-3-yl)-3-(trifluoromethyl)phenol |
| 9.24 | 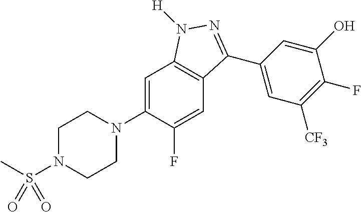 [SMILES: CS(=O)(=O)N1CCN(c2cc3[nH]nc(-c4cc(O)c(F)c(C(F)(F)F)c4)c3cc2F)CC1] | 2-Fluoro-5-(5-fluoro-6-(4- (methylsulfonyl)piperazin-1-yl)-1H-indazol-3- yl)-3-(trifluoromethyl)phenol |
| 9.25 | 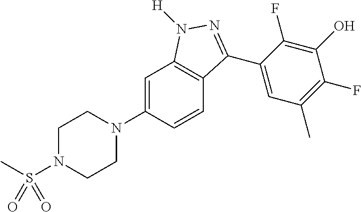 [SMILES: Cc1cc(-c2n[nH]c3cc(N4CCN(S(C)(=O)=O)CC4)ccc23)c(F)c(O)c1F] | 2,6-Difluoro-3-methyl-5-(6-(4- (methylsulfonyl)piperazin-1-yl)-1H-indazol-3- yl)phenol |
| 9.26 | 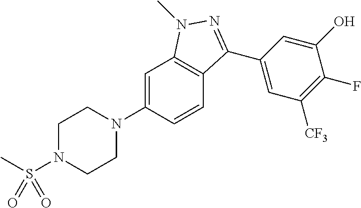 [SMILES: Cn1nc(-c2cc(O)c(F)c(C(F)(F)F)c2)c2ccc(N3CCN(S(C)(=O)=O)CC3)cc21] | 2-Fluoro-5-(1-methyl-6-(4- (methylsulfonyl)piperazin-1-yl)-1H-indazol-3- yl)-3-(trifluoromethyl)phenol |
| 9.27 | 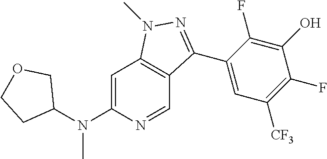 [SMILES: CN(c1cc2c(cn1)c(-c1cc(C(F)(F)F)c(F)c(O)c1F)nn2C)C1CCOC1] | 2,6-Difluoro-3-(1-methyl-6- (methyl(tetrahydrofuran-3-yl)amino)-1H- pyrazolo[4,3-c]pyridin-3-yl)-5- (trifluoromethyl)phenol |
| 9.28 | 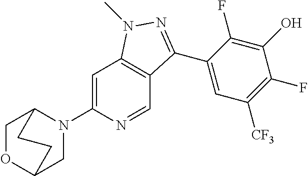 [SMILES: Cn1nc(-c2cc(C(F)(F)F)c(F)c(O)c2F)c2cnc(N3CC4CCC3CO4)cc21] | 3-(6-(2-Oxa-5-azabicylo[2.2.2]octan-5-yl)-1- methyl-1H-pyrazolo[4,3-c]pyridin-3-yl)-2,6- difluoro-5-(trifluoromethyl)phenol |
| 9.29 | 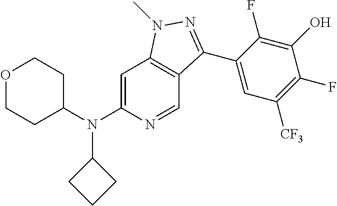 [SMILES: Cn1nc(-c2cc(C(F)(F)F)c(F)c(O)c2F)c2cnc(N(C3CCC3)C3CCOCC3)cc21] | 3-(6-(Cyclobutyl(tetrahydro-2H-pyran-4- yl)amino)-1-methyl-1H-pyrazolo[4,3-c]pyridin- 3-yl)-2,6-difluoro-5-(trifluoromethyl)phenol |
| 9.30 | 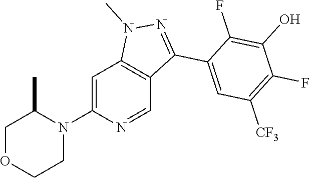 [SMILES: C[C@@H]1COCCN1c1cc2c(cn1)c(-c1cc(C(F)(F)F)c(F)c(O)c1F)nn2C] | (R)-2,6-Difluoro-3-(1-methyl-6-(3- methylmorpholino)-1H-pyrazolo[4,3-c]pyridin- 3-yl)-5-(trifluoromethyl)phenol |
| 9.31 | 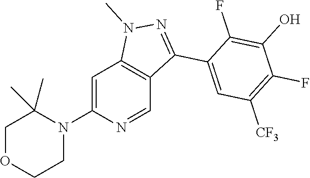 [SMILES: Cn1nc(-c2cc(C(F)(F)F)c(F)c(O)c2F)c2cnc(N3CCOCC3(C)C)cc21] | 3-(6-(3,3-Dimethylmorpholino)-1-methyl-1H- pyrazolo[4,3-c]pyridine-3-yl)-2,6-difluoro-5- (trifluoromethyl)phenol |
| 9.32 | 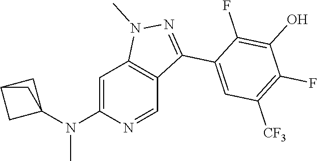 [SMILES: CN(c1cc2c(cn1)c(-c1cc(C(F)(F)F)c(F)c(O)c1F)nn2C)C12CC(C1)C2] | 3-(6-(Bicyclo[1.1.1]pentan-1-yl(methyl)amino)- 1-methyl-1H-pyrazolo[4,3-c]pyridin-3-yl)-2,6- difluoro-5-(trifluoromethyl)phenol |
| 9.33 | 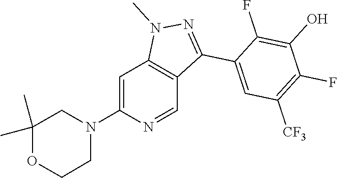 [SMILES: Cn1nc(-c2cc(C(F)(F)F)c(F)c(O)c2F)c2cnc(N3CCOC(C)(C)C3)cc21] | 3-(6-(2,2-Dimethylmorpholino)-1-methyl-1H- pyrazolo[4,3-c]pyridin-3-yl)-2,6-difluoro-5- (trifluoromethyl)phenol |
| 9.34 | 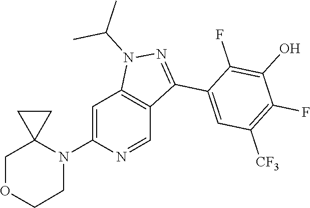 [SMILES: CC(C)n1nc(-c2cc(C(F)(F)F)c(F)c(O)c2F)c2cnc(N3CCOCC34CC4)cc21] | 2,6-Difluoro-3-(1-isopropyl-6-(7-oxa-4- azaspiro[2.5]octan-4-yl)-1H-pyrazolo[4,3- c]pyridin-3-yl)-5-(trifluoromethyl)phenol |
| 9.35 | 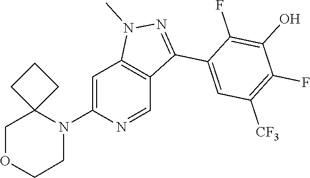 [SMILES: Cn1nc(-c2cc(C(F)(F)F)c(F)c(O)c2F)c2cnc(N3CCOCC34CCC4)cc21] | 2,6-Difluoro-3-(1-methyl-6-(8-oxa-5- azaspiro[3.5]nonan-5-yl)-1H-pyrazolo[4,3- c]pyridin-3-yl)-5-(trifluoromethyl)phenol |
| 9.36 | 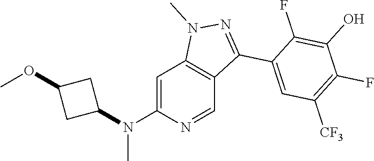 | 2,6-Difluoro-3-(6-(((1s,3s)-3- methoxycyclobutyl)(methyl)amino)-1-methyl- 1H-pyrazolo[4,3-c]pyridin-3-yl)-5- (trifluoromethyl)phenol |
| 9.37 | 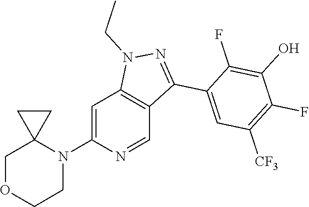 [SMILES: CCn1nc(-c2cc(C(F)(F)F)c(F)c(O)c2F)c2cnc(N3CCOCC34CC4)cc21] | 3-(1-Ethyl-6-(7-oxa-4-azaspiro[2.5]octan-4-yl)- 1H-pyrazolo[4,3-c]pyridin-3-yl)-2,6-difluoro-5- (trifluoromethyl)phenol |
| 9.38 | 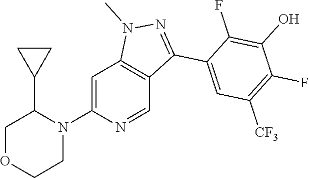 [SMILES: Cn1nc(-c2cc(C(F)(F)F)c(F)c(O)c2F)c2cnc(N3CCOCC3C3CC3)cc21] | 3-(6-(3-Cyclopropylmorpholino)-1-methyl-1H- pyrazolo[4,3-c]pyridin-3-yl)-2,6-difluoro-5- (trifluoromethyl)phenol |
| 9.39 | 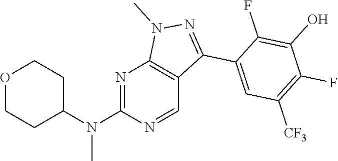 [SMILES: CN(c1ncc2c(-c3cc(C(F)(F)F)c(F)c(O)c3F)nn(C)c2n1)C1CCOCC1] | 2,6-Difluoro-3-(1-methyl-6-(methyl(tetrahydro- 2H-pyran-4-yl)amino)-1H-pyrazolo[3,4- d]pyrimidin-3-yl)-5-(trifluoromethyl)phenol |
| 9.40 | 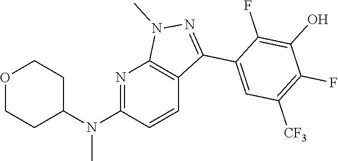 [SMILES: CN(c1ccc2c(-c3cc(C(F)(F)F)c(F)c(O)c3F)nn(C)c2n1)C1CCOCC1] | 2,6-Difluoro-3-(1-methyl-6-(methyl(tetrahydro- 2H-pyran-4-yl)amino)-1H-pyrazolo[3,4- b]pyridin-3-yl)-5-(trifluoromethyl)phenol |
| 9.41 | 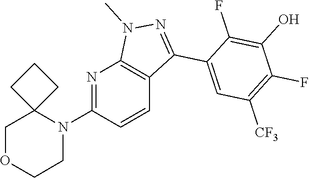 [SMILES: Cn1nc(-c2cc(C(F)(F)F)c(F)c(O)c2F)c2ccc(N3CCOCC34CCC4)nc21] | 2,6-Difluoro-3-(1-methyl-6-(8-oxa-5- azaspiro[3.5]nonan-5-yl)-1H-pyrazolo[3,4- b]pyridin-3-yl)-5-(trifluoromethyl)phenol |
| 9.42 | 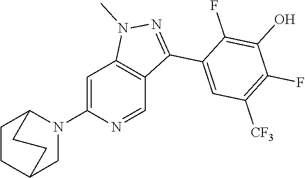 [SMILES: Cn1nc(-c2cc(C(F)(F)F)c(F)c(O)c2F)c2cnc(N3CC4CCC3CC4)cc21] | 3-(6-(2-Azabicyclo[2.2.2]octan-2-yl)-1-methyl- 1H-pyrazolo[4,3-c]pyridin-3-yl)-2,6-difluoro-5- (trifluoromethyl)phenol |
| 9.43 | 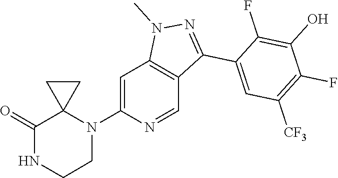 [SMILES: Cn1nc(-c2cc(C(F)(F)F)c(F)c(O)c2F)c2cnc(N3CCNC(=O)C34CC4)cc21] | 4-(3-(2,4-Difluoro-3-hydroxy-5- (trifluoromethyl)phenyl)-1-methyl-1H- pyrazolo[4,3-c]pyridin-6-yl)-4,7- diazaspiro[2.5]octan-8-one |
| 9.44 | 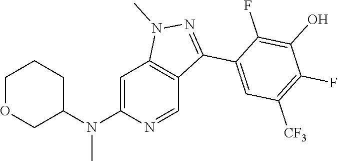 [SMILES: CN(c1cc2c(cn1)c(-c1cc(C(F)(F)F)c(F)c(O)c1F)nn2C)C1CCCOC1] | 2,6-Difluoro-3-(1-methyl-6-(methyl(tetrahydro- 2H-pyran-3-yl)amino)-1H-pyrazolo[4,3- c]pyridin-3-yl)-5-(trifluoromethyl)phenol |
| 9.45 | 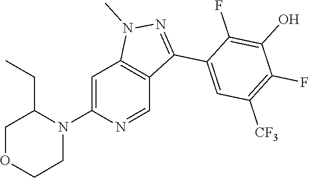 [SMILES: CCC1COCCN1c1cc2c(cn1)c(-c1cc(C(F)(F)F)c(F)c(O)c1F)nn2C] | 3-(6-(3-Ethylmorpholino)-1-methyl-1H- pyrazolo[4,3-c]pyridin-3-yl)-2,6-difluoro-5- (trifluoromethyl)phenol |
| 9.46 | 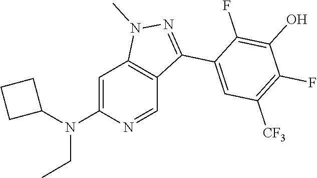 [SMILES: CCN(c1cc2c(cn1)c(-c1cc(C(F)(F)F)c(F)c(O)c1F)nn2C)C1CCC1] | 3-(6-(Cyclobutyl(ethyl)amino)-1-methyl-1H- pyrazolo[4,3-c]pyridin-3-yl)-2,6-difluoro-5- (trifluoromethyl)phenol |
| 9.47 | 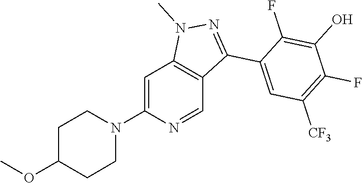 [SMILES: COC1CCN(c2cc3c(cn2)c(-c2cc(C(F)(F)F)c(F)c(O)c2F)nn3C)CC1] | 2,6-Difluoro-3-(6-(4-methoxypiperidin-1-yl)-1- methyl-1H-pyrazolo[4,3-c]pyridin-3-yl)-5- (trifluoromethyl)phenol |
| 9.48 | 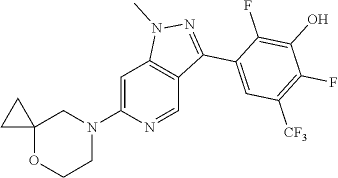 [SMILES: Cn1nc(-c2cc(C(F)(F)F)c(F)c(O)c2F)c2cnc(N3CCOC4(CC4)C3)cc21] | 2,6-Difluoro-3-(1-methyl-6-(4-oxa-7- azaspiro[2.5]octan-7-yl)-1H-pyrazolo[4,3- c]pyridin-3-yl)-5-(trifluoromethyl)phenol |
| 9.49 | 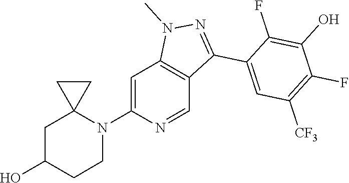 [SMILES: Cn1nc(-c2cc(C(F)(F)F)c(F)c(O)c2F)c2cnc(N3CCC(O)CC34CC4)cc21] | 4-(3-(2,4-Difluoro-3-hydroxy-5- (trifluoromethyl)phenyl)-1-methyl-1H- pyrazolo[4,3-c]pyridin-6-yl)-4- azaspiro[2.5]octan-7-ol |
| 9.50 | 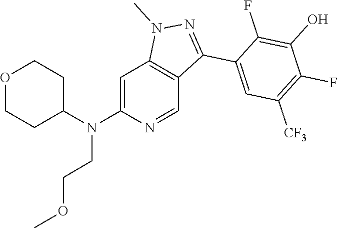 [SMILES: COCCN(c1cc2c(cn1)c(-c1cc(C(F)(F)F)c(F)c(O)c1F)nn2C)C1CCOCC1] | 2,6-Difluoro-3-(6-((2- methoxyethyl)(tetrahydro-2H-pyran-4- yl)amino)-1-methyl-1H-pyrazolo[4,3-c]pyridin- 3-yl)-5-(trifluoromethyl)phenol |
| 9.51 | 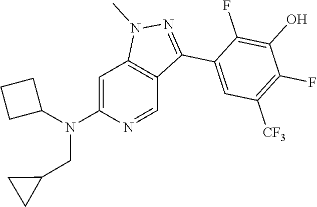 [SMILES: Cn1nc(-c2cc(C(F)(F)F)c(F)c(O)c2F)c2cnc(N(CC3CC3)C3CCC3)cc21] | 3-(6-(Cyclobutyl(cyclopropylmethyl)amino)-1- methyl-1H-pyrazolo[4,3-c]pyridin-3-yl)-2,6- difluoro-5-(trifluoromethyl)phenol |
| 9.52 | 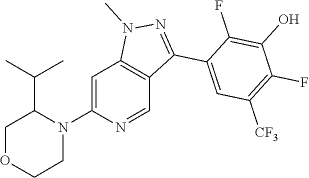 [SMILES: CC(C)C1COCCN1c1cc2c(cn1)c(-c1cc(C(F)(F)F)c(F)c(O)c1F)nn2C] | 2,6-Difluoro-3-(6-(3-isopropylmorpholino)-1- methyl-1H-pyrazolo[4,3-c]pyridin-3-yl)-5- (trifluoromethyl)phenol |
| 9.53 | 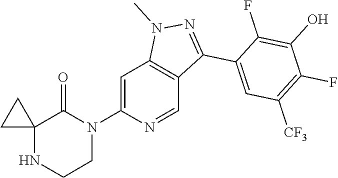 [SMILES: Cn1nc(-c2cc(C(F)(F)F)c(F)c(O)c2F)c2cnc(N3CCNC4(CC4)C3=O)cc21] | 7-(3-(2,4-Difluoro-3-hydroxy-5- (trifluoromethyl)phenyl)-1-methyl-1H- pyrazolo[4,3-c]pyridin-6-yl)-4,7- diazaspiro[2.5]octan-8-one |
| 9.54 | 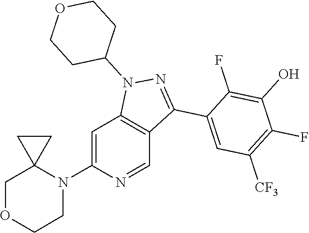 [SMILES: Oc1c(F)c(-c2nn(C3CCOCC3)c3cc(N4CCOCC45CC5)ncc23)cc(C(F)(F)F)c1F] | 3-(6-(7-Oxa-4-azaspiro[2.5]octan-4-yl)-1- (tetrahydro-2H-pyran-4-yl)-1H-pyrazolo[4,3- c]pyridin-3-yl)-2,6-difluoro-5- (trifluoromethyl)phenol |
| 9.55 | 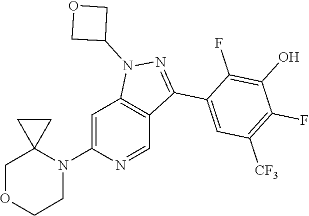 [SMILES: Oc1c(F)c(-c2nn(C3COC3)c3cc(N4CCOCC45CC5)ncc23)cc(C(F)(F)F)c1F] | 2,6-Difluoro-3-(1-(oxetan-3-yl)-6-(7-oxa-4- azaspiro[2.5]octan-4-yl)-1H-pyrazolo[4,3- c]pyridin-3-yl)-5-(trifluoromethyl)phenol |
| 9.56 | 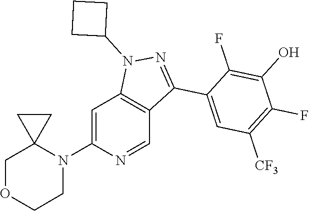 [SMILES: Oc1c(F)c(-c2nn(C3CCC3)c3cc(N4CCOCC45CC5)ncc23)cc(C(F)(F)F)c1F] | 3-(1-Cyclobutyl-6-(7-oxa-4-azaspiro[2.5]octan- 4-yl)-1H-pyrazolo[4,3-c]pyridin-3-yl)-2,6- difluoro-5-(trifluoromethyl)phenol |
| 9.57 | 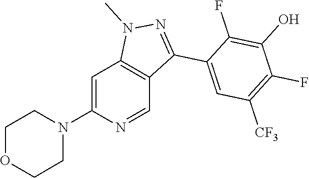 [SMILES: Cn1nc(-c2cc(C(F)(F)F)c(F)c(O)c2F)c2cnc(N3CCOCC3)cc21] | 2,6-Difluoro-3-(1-methyl-6-morpholino-1H- pyrazolo[4,3-c]pyridin-3-yl)-5- (trifluoromethyl)phenol |
| 9.58 | 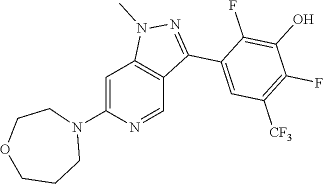 [SMILES: Cn1nc(-c2cc(C(F)(F)F)c(F)c(O)c2F)c2cnc(N3CCCOCC3)cc21] | 2,6-Difluoro-3-(1-methyl-6-(1,4-oxazepan-4- yl)-1H-pyrazolo[4,3-c]pyridin-3-yl)-5- (trifluoromethyl)phenol |
| 9.59 | 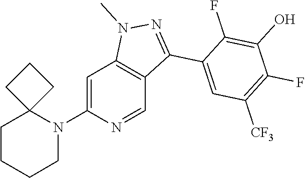 [SMILES: Cn1nc(-c2cc(C(F)(F)F)c(F)c(O)c2F)c2cnc(N3CCCCC34CCC4)cc21] | 2,6-Difluoro-3-(1-methyl-6-(5- azaspiro[3.5]nonan-5-yl)-1H-pyrazolo[4,3- c]pyridin-3-yl)-5-(trifluoromethyl)phenol |
| 9.60 | 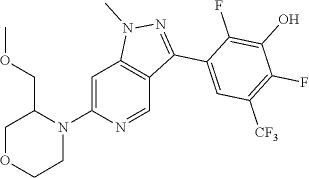 [SMILES: COCC1COCCN1c1cc2c(cn1)c(-c1cc(C(F)(F)F)c(F)c(O)c1F)nn2C] | 2,6-Difluoro-3-(6-(3- (methoxymethyl)morpholino)-1-methyl-1H- pyrazolo[4,3-c]pyridin-3-yl)-5- (trifluoromethyl)phenol |
| 9.61 | 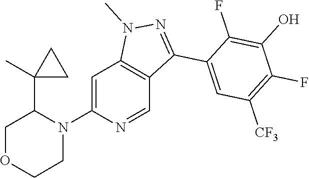 [SMILES: Cn1nc(-c2cc(C(F)(F)F)c(F)c(O)c2F)c2cnc(N3CCOCC3C3(C)CC3)cc21] | 2,6-Difluoro-3-(1-methyl-6-(3-(1- methylcyclopropyl)morpholino)-1H- pyrazolo[4,3-c]pyridin-3-yl)-5- (trifluoromethyl)phenol |
| 9.62 | 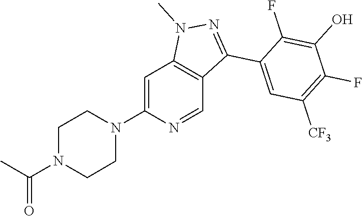 [SMILES: CC(=O)N1CCN(c2cc3c(cn2)c(-c2cc(C(F)(F)F)c(F)c(O)c2F)nn3C)CC1] | 1-(4-(3-(2,4-Difluoro-3-hydroxy-5- (trifluoromethyl)phenyl)-1-methyl-1H- pyrazolo[4,3-c]pyridin-6-yl)piperazin-1- yl)ethanone |
| 9.63 | 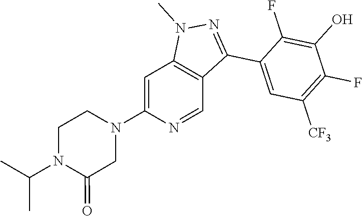 [SMILES: CC(C)N1CCN(c2cc3c(cn2)c(-c2cc(C(F)(F)F)c(F)c(O)c2F)nn3C)CC1=O] | 4-(3-(2,4-Difluoro-3-hydroxy-5- (trifluoromethyl)phenyl)-1-methyl-1H- pyrazolo[4,3-c]pyridin-6-yl)-1- isopropylpiperazin-2-one |
| 9.64 | 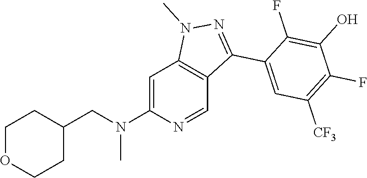 [SMILES: CN(CC1CCOCC1)c1cc2c(cn1)c(-c1cc(C(F)(F)F)c(F)c(O)c1F)nn2C] | 2,6-Difluoro-3-(1-methyl-6- (methyl((tetrahydro-2H-pyran-4- yl)methyl)amino)-1H-pyrazolo[4,3-c]pyridin-3- yl)-5-(trifluoromethyl)phenol |
| 9.65 | 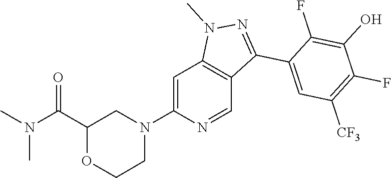 [SMILES: CN(C)C(=O)C1CN(c2cc3c(cn2)c(-c2cc(C(F)(F)F)c(F)c(O)c2F)nn3C)CCO1] | 4-(3-(2,4-Difluoro-3-hydroxy-5- (trifluoromethyl)phenyl)-1-methyl-1H- pyrazolo[4,3-c]pyridin-6-yl)-N,N- dimethylmorpholine-2-carboxamide |
| 9.66 | 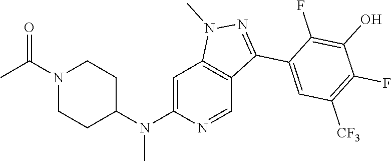 [SMILES: CC(=O)N1CCC(N(C)c2cc3c(cn2)c(-c2cc(C(F)(F)F)c(F)c(O)c2F)nn3C)CC1] | 1-(4-((3-(2,4-Difluoro-3-hydroxy-5- (trifluoromethyl)phenyl)-1-methyl-1H- pyrazolo[4,3-c]pyridin-6- yl)(methyl)amino)piperidin-1-yl)ethanone |
| 9.67 | 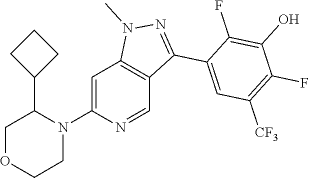 [SMILES: Cn1nc(-c2cc(C(F)(F)F)c(F)c(O)c2F)c2cnc(N3CCOCC3C3CCC3)cc21] | 3-(6-(3-Cyclobutylmorpholino)-1-methyl-1H- pyrazolo[4,3-c]pyridin-3-yl)-2,6-difluoro-5- (trifluoromethyl)phenol |
| 9.68 | 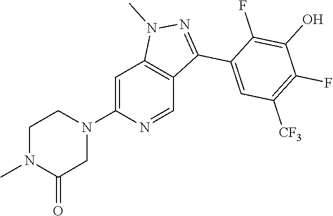 [SMILES: CN1CCN(c2cc3c(cn2)c(-c2cc(C(F)(F)F)c(F)c(O)c2F)nn3C)CC1=O] | 4-(3-(2,4-Difluoro-3-hydroxy-5- (trifluoromethyl)phenyl)-1-methyl-1H- pyrazolo[4,3-c]pyridin-6-yl)-1-methylpiperazin- 2-one |
| 9.69 | 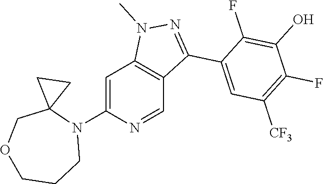 [SMILES: Cn1nc(-c2cc(C(F)(F)F)c(F)c(O)c2F)c2cnc(N3CCCOCC34CC4)cc21] | 2,6-Difluoro-3-(1-methyl-6-(8-oxa-4- azaspiro[2.6]nonan-4-yl)-1H-pyrazolo[4,3- c]pyridin-3-yl)-5-(trifluoromethyl)phenol |
| 9.70 | 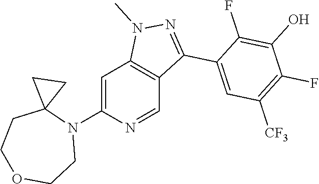 [SMILES: Cn1nc(-c2cc(C(F)(F)F)c(F)c(O)c2F)c2cnc(N3CCOCCC34CC4)cc21] | 2,6-Difluoro-3-(1-methyl-6-(7-oxa-4- azaspiro[2.6]nonan-4-yl)-1H-pyrazolo[4,3- c]pyridin-3-yl)-5-(trifluoromethyl)phenol |
| 9.71 | 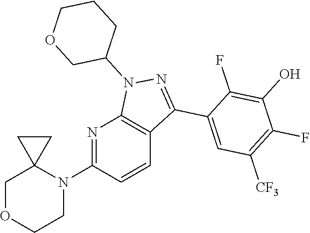 [SMILES: Oc1c(F)c(-c2nn(C3CCCOC3)c3nc(N4CCOCC45CC5)ccc23)cc(C(F)(F)F)c1F] | 3-(6-(7-Oxa-4-azaspiro[2.5]octan-4-yl)-1- (tetrahydro-2H-pyran-3-yl)-1H-pyrazolo[3,4- b]pyridin-3-yl)-2,6-difluoro-5- (trifluoromethyl)phenol |
| 9.72 | 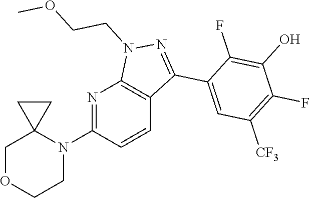 [SMILES: COCCn1nc(-c2cc(C(F)(F)F)c(F)c(O)c2F)c2ccc(N3CCOCC34CC4)nc21] | 2,6-Difluoro-3-(1-(2-methoxyethyl)-6-(7-oxa-4- azaspiro[2.5]octan-4-yl)-1H-pyrazolo[3,4- b]pyridin-3-yl)-5-(trifluoromethyl)phenol |
| 9.73 | 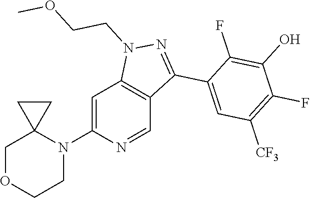 [SMILES: COCCn1nc(-c2cc(C(F)(F)F)c(F)c(O)c2F)c2cnc(N3CCOCC34CC4)cc21] | 2,6-Difluoro-3-(1-(2-methoxyethyl)-6-(7-oxa-4- azaspiro[2.5]octan-4-yl)-1H-pyrazolo[4,3- c]pyridin-3-yl)-5-(trifluoromethyl)phenol |
| 9.74 | 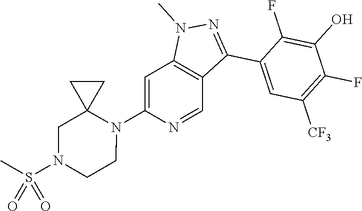 [SMILES: Cn1nc(-c2cc(C(F)(F)F)c(F)c(O)c2F)c2cnc(N3CCN(S(C)(=O)=O)CC34CC4)cc21] | 2,6-Difluoro-3-(1-methyl-6-(7- (methylsulfonyl)-4,7-diazaspiro[2.5]octan-4-yl)- 1H-pyrazolo[4,3-c]pyridin-3-yl)-5- (trifluoromethyl)phenol |
| 9.75 | 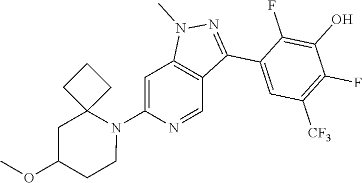 [SMILES: COC1CCN(c2cc3c(cn2)c(-c2cc(C(F)(F)F)c(F)c(O)c2F)nn3C)C2(CCC2)C1] | 2,6-Difluoro-3-(6-(8-methoxy-5- azaspiro[3.5]nonan-5-yl)-1-methyl-1H- pyrazolo[4,3-c]pyridin-3-yl)-5- (trifluoromethyl)phenol |
| 9.76 | 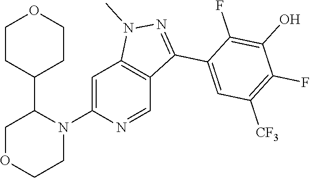 [SMILES: Cn1nc(-c2cc(C(F)(F)F)c(F)c(O)c2F)c2cnc(N3CCOCC3C3CCOCC3)cc21] | 2,6-Difluoro-3-(1-methyl-6-(3-(tetrahydro-2H- pyran-4-yl)morpholino)-1H-pyrazolo[4,3- c]pyridin-3-yl)-5-(trifluoromethyl)phenol |
| 9.77 | 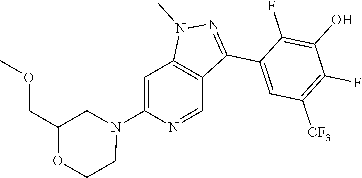 [SMILES: COCC1CN(c2cc3c(cn2)c(-c2cc(C(F)(F)F)c(F)c(O)c2F)nn3C)CCO1] | 2,6-Difluoro-3-(6-(2- (methoxymethyl)morpholino)-1-methyl-1H- pyrazolo[4,3-c]pyridin-3-yl)-5- (trifluoromethyl)phenol |
| 9.78 | 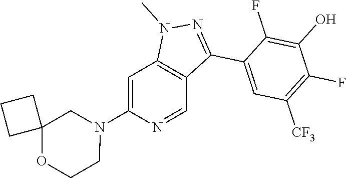 [SMILES: Cn1nc(-c2cc(C(F)(F)F)c(F)c(O)c2F)c2cnc(N3CCOC4(CCC4)C3)cc21] | 2,6-Difluoro-3-(1-methyl-6-(5-oxa-8- azaspiro[3.5]nonan-8-yl)-1H-pyrazolo[4,3- c]pyridin-3-yl)-5-(trifluoromethyl)phenol |
| 9.79 | 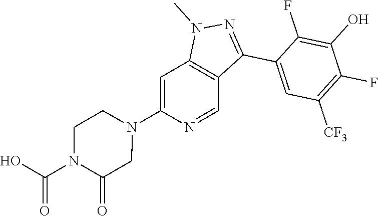 [SMILES: Cn1nc(-c2cc(C(F)(F)F)c(F)c(O)c2F)c2cnc(N3CCN(C(=O)O)C(=O)C3)cc21] | 4-(3-(2,4-Difluoro-3-hydroxy-5- (trifluoromethyl)phenyl)-1-methyl-1H- pyrazolo[4,3-c]pyridin-6-yl)-2-oxopiperazine-1- carboxylic acid |
| 9.80 | 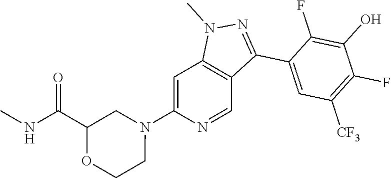 [SMILES: CNC(=O)C1CN(c2cc3c(cn2)c(-c2cc(C(F)(F)F)c(F)c(O)c2F)nn3C)CCO1] | 4-(3-(2,4-Difluoro-3-hydroxy-5- (trifluoromethyl)phenyl)-1-methyl-1H- pyrazolo[4,3-c]pyridin-6-yl)-N- methylmorpholine-2-carboxamide |
| 9.81 | 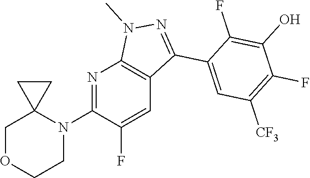 [SMILES: Cn1nc(-c2cc(C(F)(F)F)c(F)c(O)c2F)c2cc(F)c(N3CCOCC34CC4)nc21] | 2,6-Difluoro-3-(5-fluoro-1-methyl-6-(7-oxa-4- azaspiro[2.5]octan-4-yl)-1H-pyrazolo[3,4- b]pyridin-3-yl)-5-(trifluoromethyl)phenol |
| 9.82 | 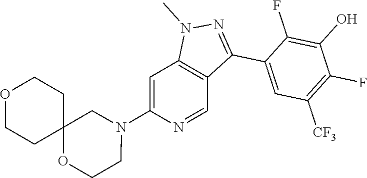 [SMILES: Cn1nc(-c2cc(C(F)(F)F)c(F)c(O)c2F)c2cnc(N3CCOC4(CCOCC4)C3)cc21] | 2,6-Difluoro-3-(1-methyl-6-(1,9-dioxa-4- azaspiro[5.5]undecan-4-yl)-1H-pyrazolo[4,3- c]pyridin-3-yl)-5-(trifluoromethyl)phenol |
| 9.83 | 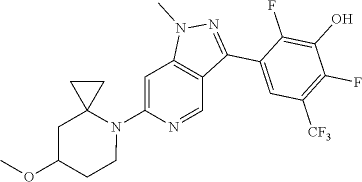 [SMILES: COC1CCN(c2cc3c(cn2)c(-c2cc(C(F)(F)F)c(F)c(O)c2F)nn3C)C2(CC2)C1] | 2,6-Difluoro-3-(6-(7-methoxy-4- azaspiro[2.5]octan-4-yl)-1-methyl-1H- pyrazolo[4,3-c]pyridin-3-yl)-5- (trifluoromethyl)phenol |
| 9.84 | 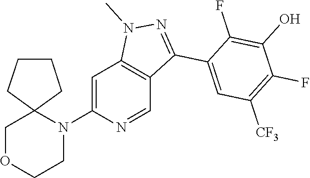 [SMILES: Cn1nc(-c2cc(C(F)(F)F)c(F)c(O)c2F)c2cnc(N3CCOCC34CCCC4)cc21] | 2,6-Difluoro-3-(1-methyl-6-(9-oxa-6- azaspiro[4.5]decan-6-yl)-1H-pyrazolo[4,3- c]pyridin-3-yl)-5-(trifluoromethyl)phenol |
| 9.85 | 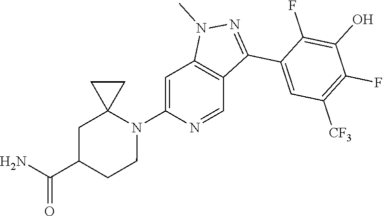 [SMILES: Cn1nc(-c2cc(C(F)(F)F)c(F)c(O)c2F)c2cnc(N3CCC(C(N)=O)CC34CC4)cc21] | 4-(3-(2,4-Difluoro-3-hydroxy-5- (trifluoromethyl)phenyl)-1-methyl-1H- pyrazolo[4,3-c]pyridin-6-yl)-4- azaspiro[2.5]octane-7-carboxamide |
| 9.86 | 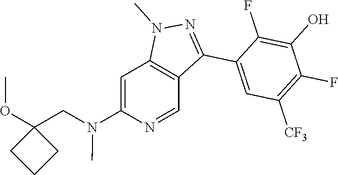 [SMILES: COC1(CN(C)c2cc3c(cn2)c(-c2cc(C(F)(F)F)c(F)c(O)c2F)nn3C)CCC1] | 2,6-Difluoro-3-(6-(((1- methoxycyclobutyl)methyl)(methyl)amino)-1- methyl-1H-pyrazolo[4,3-c]pyridin-3-yl)-5- (trifluoromethyl)phenol |
| 9.87 | 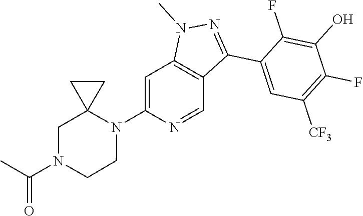 [SMILES: CC(=O)N1CCN(c2cc3c(cn2)c(-c2cc(C(F)(F)F)c(F)c(O)c2F)nn3C)C2(CC2)C1] | 1-(4-(3-(2,4-Difluoro-3-hydroxy-5- (trifluoromethyl)phenyl)-1-methyl-1H- pyrazolo[4,3-c]pyridin-6-yl)-4,7- diazaspiro[2.5]octan-7-yl)ethanone |
| 9.88 | 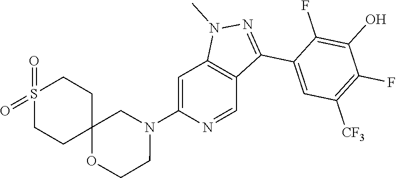 [SMILES: Cn1nc(-c2cc(C(F)(F)F)c(F)c(O)c2F)c2cnc(N3CCOC4(CCS(=O)(=O)CC4)C3)cc21] | 4-(3-(2,4-Difluoro-3-hydroxy-5- (trifluoromethyl)phenyl)-1-methyl-1H- pyrazolo[4,3-c]pyridin-6-yl)-1-oxa-9-thia-4- azaspiro[5.5]undecane-9,9-dioxide |
| 9.89 | 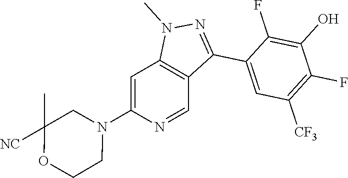 [SMILES: Cn1nc(-c2cc(C(F)(F)F)c(F)c(O)c2F)c2cnc(N3CCOC(C)(C#N)C3)cc21] | 4-(3-(2,4-Difluoro-3-hydroxy-5- (trifluoromethyl)phenyl)-1-methyl-1H- pyrazolo[4,3-c]pyridin-6-yl)-2- methylmorpholine-2-carbonitrile |
| 9.90 | 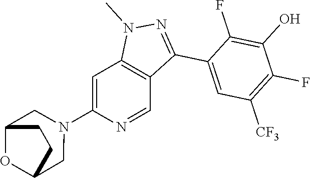 [SMILES: Cn1nc(-c2cc(C(F)(F)F)c(F)c(O)c2F)c2cnc(N3C[C@H]4CC[C@@H](C3)O4)cc21] | 3-(6-(cis-8-Oxa-3-azabicyclo[3.2.1]octan-3-yl)- 1-methyl-1H-pyrazolo[4,3-c]pyridin-3-yl)-2,6- difluoro-5-(trifluoromethyl)phenol |
| 9.91 | 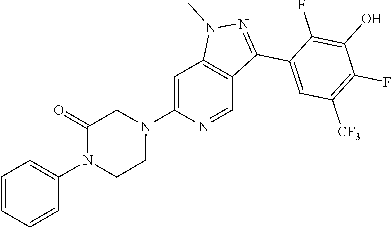 [SMILES: Cn1nc(-c2cc(C(F)(F)F)c(F)c(O)c2F)c2cnc(N3CCN(c4ccccc4)C(=O)C3)cc21] | 4-(3-(2,4-Difluoro-3-hydroxy-5- (trifluoromethyl)phenyl)-1-methyl-1H- pyrazolo[4,3-c]pyridin-6-yl)-1-phenylpiperazin- 2-one |
| 9.92 | 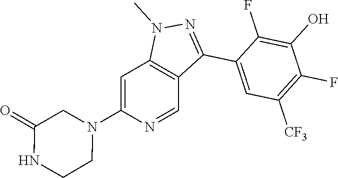 [SMILES: Cn1nc(-c2cc(C(F)(F)F)c(F)c(O)c2F)c2cnc(N3CCNC(=O)C3)cc21] | 4-(3-(2,4-Difluoro-3-hydroxy-5- (trifluoromethyl)phenyl)-1-methyl-1H- pyrazolo[4,3-c]pyridin-6-yl)piperazin-2-one |
| 9.93 | 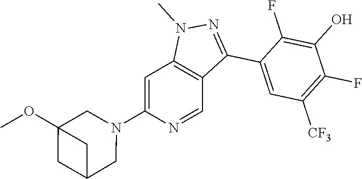 [SMILES: COC12CC(CN(c3cc4c(cn3)c(-c3cc(C(F)(F)F)c(F)c(O)c3F)nn4C)C1)C2] | 2,6-Difluoro-3-(6-(1-methoxy-3- azabicyclo[3.1.1]heptan-3-yl)-1-methyl-1H- pyrazolo[4,3-c]pyridin-3-yl)-5- (trifluoromethyl)phenol |
| 9.94 | 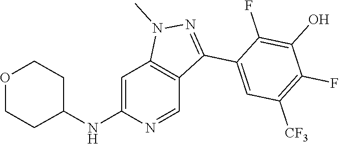 [SMILES: Cn1nc(-c2cc(C(F)(F)F)c(F)c(O)c2F)c2cnc(NC3CCOCC3)cc21] | 2,6-Difluoro-3-(1-methyl-6-((tetrahydro-2H- pyran-4-yl)amino)-1H-pyrazolo[4,3-c]pyridin- 3-yl)-5-(trifluoromethyl)phenol |
| 9.95 | 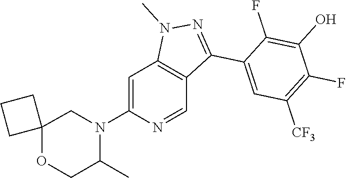 [SMILES: CC1COC2(CCC2)CN1c1cc2c(cn1)c(-c1cc(C(F)(F)F)c(F)c(O)c1F)nn2C] | 2,6-Difluoro-3-(1-methyl-6-(7-methyl-5-oxa-8- azaspiro[3.5]nonan-8-yl)-1H-pyrazolo[4,3- c]pyridin-3-yl)-5-(trifluoromethyl)phenol |
| 9.96 | 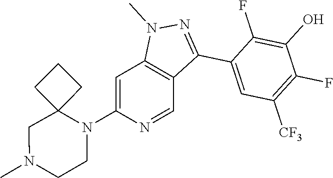 [SMILES: CN1CCN(c2cc3c(cn2)c(-c2cc(C(F)(F)F)c(F)c(O)c2F)nn3C)C2(CCC2)C1] | 2,6-Difluoro-3-(1-methyl-6-(8-methyl-5,8- diazaspiro[3.5]nonan-5-yl)-1H-pyrazolo[4,3- c]pyridin-3-yl)-5-(trifluoromethyl)phenol |
| 9.97 | 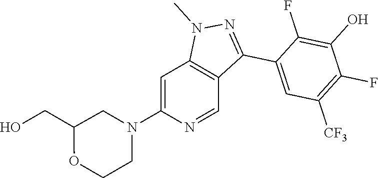 [SMILES: Cn1nc(-c2cc(C(F)(F)F)c(F)c(O)c2F)c2cnc(N3CCOC(CO)C3)cc21] | 2,6-Difluoro-3-(6-(2- (hydroxymethyl)morpholino)-1-methyl-1H- pyrazolo[4,3-c]pyridin-3-yl)-5- (trifluoromethyl)phenol |
| 9.98 | 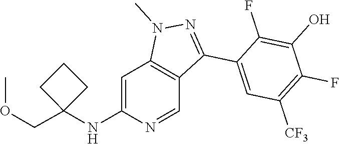 [SMILES: COCC1(Nc2cc3c(cn2)c(-c2cc(C(F)(F)F)c(F)c(O)c2F)nn3C)CCC1] | 2,6-Difluoro-3-(6-((1- (methoxymethyl)cyclobutyl)amino)-1-methyl- 1H-pyrazolo[4,3-c]pyridin-3-yl)-5- (trifluoromethyl)phenol |
| 9.99 | 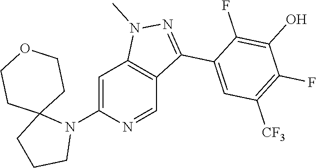 [SMILES: Cn1nc(-c2cc(C(F)(F)F)c(F)c(O)c2F)c2cnc(N3CCCC34CCOCC4)cc21] | 2,6-Difluoro-3-(1-methyl-6-(8-oxa-1- azaspiro[4.5]decan-1-yl)-1H-pyrazolo[4,3- c]pyridin-3-yl)-5-(trifluoromethyl)phenol |
| 9.100 | 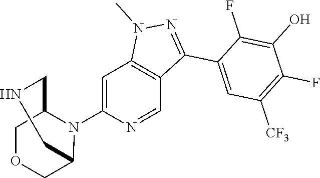 [SMILES: Cn1nc(-c2cc(C(F)(F)F)c(F)c(O)c2F)c2cnc(N3[C@@H]4CNC[C@H]3COC4)cc21] | 3-(6-(cis-3-Oxa-7,9-diazabicyclo[3.3.1]nonan- 9-yl)-1-methyl-1H-pyrazolo[4,3-c]pyridin-3- yl)-2,6-difluoro-5-(trifluoromethyl)phenol |
| 9.101 | 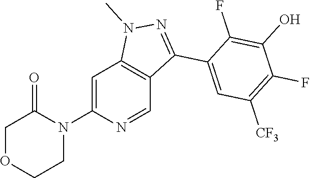 [SMILES: Cn1nc(-c2cc(C(F)(F)F)c(F)c(O)c2F)c2cnc(N3CCOCC3=O)cc21] | 4-(3-(2,4-Difluoro-3-hydroxy-5- (trifluoromethyl)phenyl)-1-methyl-1H- pyrazolo[4,3-c]pyridin-6-yl)morpholin-3-one |
| 9.102 | 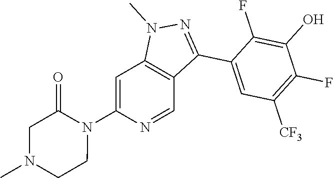 [SMILES: CN1CCN(c2cc3c(cn2)c(-c2cc(C(F)(F)F)c(F)c(O)c2F)nn3C)C(=O)C1] | 1-(3-(2,4-Difluoro-3-hydroxy-5- (trifluoromethyl)phenyl)-1-methyl-1H- pyrazolo[4,3-c]pyridin-6-yl)-4-methylpiperazin- 2-one |
| 9.103 | 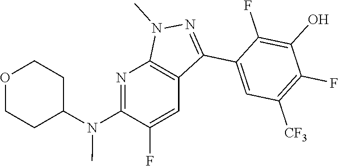 [SMILES: CN(c1nc2c(cc1F)c(-c1cc(C(F)(F)F)c(F)c(O)c1F)nn2C)C1CCOCC1] | 2,6-Difluoro-3-(5-fluoro-1-methyl-6- (methyl(tetrahydro-2H-pyran-4-yl)amino)-1H- pyrazolo[3,4-b]pyridin-3-yl)-5- (trifluoromethyl)phenol |
| 9.104 | 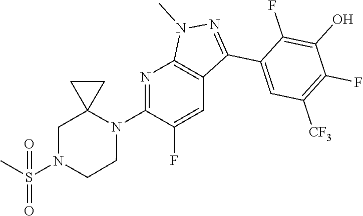 [SMILES: Cn1nc(-c2cc(C(F)(F)F)c(F)c(O)c2F)c2cc(F)c(N3CCN(S(C)(=O)=O)CC34CC4)nc21] | 2,6-Difluoro-3-(5-fluoro-1-methyl-6-(7- (methylsulfonyl)-4,7-diazaspiro[2.5]octan-4-yl)- 1H-pyrazolo[3,4-b]pyridin-3-yl)-5- (trifluoromethyl)phenol |
| 9.105 | 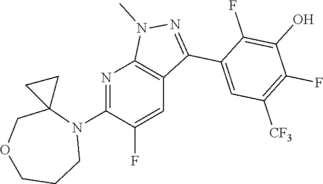 [SMILES: Cn1nc(-c2cc(C(F)(F)F)c(F)c(O)c2F)c2cc(F)c(N3CCCOCC34CC4)nc21] | 2,6-Difluoro-3-(5-fluoro-1-methyl-6-(8-oxa-4- azaspiro[2.6]nonan-4-yl)-1H-pyrazolo[3,4- b]pyridin-3-yl)-5-(trifluoromethyl)phenol |
| 9.106 | 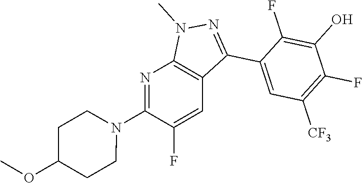 [SMILES: COC1CCN(c2nc3c(cc2F)c(-c2cc(C(F)(F)F)c(F)c(O)c2F)nn3C)CC1] | 2,6-Difluoro-3-(5-fluoro-6-(4- methoxypiperidin-1-yl)-1-methyl-1H- pyrazolo[3,4-b]pyridin-3-yl)-5- (trifluoromethyl)phenol |
| 9.107 | 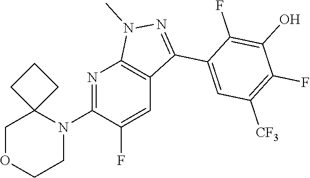 [SMILES: Cn1nc(-c2cc(C(F)(F)F)c(F)c(O)c2F)c2cc(F)c(N3CCOCC34CCC4)nc21] | 2,6-Difluoro-3-(5-fluoro-1-methyl-6-(8-oxa-5- azaspiro[3.5]nonan-5-yl)-1H-pyrazolo[3,4- b]pyridin-3-yl)-5-(trifluoromethyl)phenol |
| 9.108 | 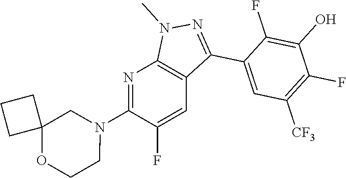 [SMILES: Cn1nc(-c2cc(C(F)(F)F)c(F)c(O)c2F)c2cc(F)c(N3CCOC4(CCC4)C3)nc21] | 2,6-Difluoro-3-(5-fluoro-1-methyl-6-(5-oxa-8- azaspiro[3.5]nonan-8-yl)-1H-pyrazolo[3,4- b]pyridin-3-yl)-5-(trifluoromethyl)phenol |
| 9.109 | 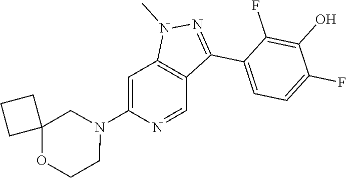 [SMILES: Cn1nc(-c2ccc(F)c(O)c2F)c2cnc(N3CCOC4(CCC4)C3)cc21] | 2,6-Difluoro-3-(1-methyl-6-(5-oxa-8- azaspiro[3.5]nonan-8-yl)-1H-pyrazolo[4,3- c]pyridin-3-yl)phenol |
| 9.110 | 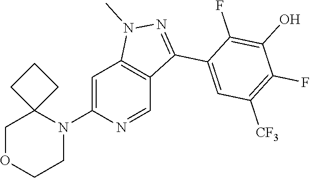 [SMILES: Cn1nc(-c2cc(C(F)(F)F)c(F)c(O)c2F)c2cnc(N3CCOCC34CCC4)cc21] | 2,6-Difluoro-3-(1-methyl-6-(8-oxa-5- azaspiro[3.5]nonan-5-yl)-1H-pyrazolo[3,4- d]pyrimidin-3-yl)-5-(trifluoromethyl)phenol |
| 9.111 | 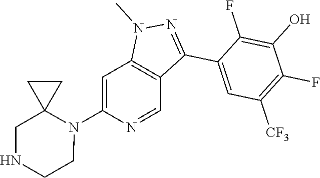 [SMILES: Cn1nc(-c2cc(C(F)(F)F)c(F)c(O)c2F)c2cnc(N3CCNCC34CC4)cc21] | 2,6-Difluoro-3-(1-methyl-6-(4,7- diazaspiro[2.5]octan-4-yl)-1H-pyrazolo[4,3- c]pyridin-3-yl)-5-(trifluoromethyl)phenol |
| 9.112 | 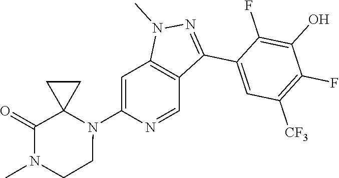 [SMILES: CN1CCN(c2cc3c(cn2)c(-c2cc(C(F)(F)F)c(F)c(O)c2F)nn3C)C2(CC2)C1=O] | 4-(3-(2,4-Difluoro-3-hydroxy-5- (trifluoromethyl)phenyl)-1-methyl-1H- pyrazolo[4,3-c]pyridin-6-yl)-7-methyl-4,7- diazaspiro[2.5]octan-8-one |
| 9.113 | 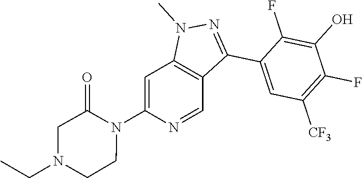 [SMILES: CCN1CCN(c2cc3c(cn2)c(-c2cc(C(F)(F)F)c(F)c(O)c2F)nn3C)C(=O)C1] | 1-(3-(2,4-Difluoro-3-hydroxy-5- (trifluoromethyl)phenyl)-1-methyl-1H- pyrazolo[4,3-c]pyridin-6-yl)-4-ethylpiperazin-2- one |
| 9.114 | 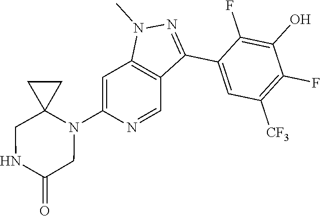 [SMILES: Cn1nc(-c2cc(C(F)(F)F)c(F)c(O)c2F)c2cnc(N3CC(=O)NCC34CC4)cc21] | 4-(3-(2,4-Difluoro-3-hydroxy-5- (trifluoromethyl)phenyl)-1-methyl-1H- pyrazolo[4,3-c]pyridin-6-yl)-4,7- diazaspiro[2.5]octan-6-one |
| 9.115 | 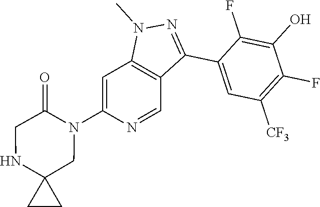 [SMILES: Cn1nc(-c2cc(C(F)(F)F)c(F)c(O)c2F)c2cnc(N3CC4(CC4)NCC3=O)cc21] | 7-(3-(2,4-Difluoro-3-hydroxy-5- (trifluoromethyl)phenyl)-1-methyl-1H- pyrazolo[4,3-c]pyridin-6-yl)-4,7- diazaspiro[2.5]octan-6-one |
| 9.116 | 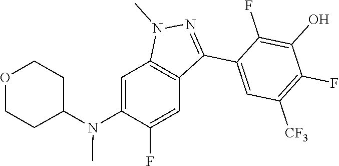 [SMILES: CN(c1cc2c(cc1F)c(-c1cc(C(F)(F)F)c(F)c(O)c1F)nn2C)C1CCOCC1] | 2,6-Difluoro-3-(5-fluoro-1-methyl-6- (methyl(tetrahydro-2H-pyran-4-yl)amino)-1H- indazol-3-yl)-5-(trifluoromethyl)phenol |
| 9.117 | 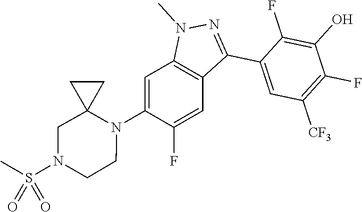 [SMILES: Cn1nc(-c2cc(C(F)(F)F)c(F)c(O)c2F)c2cc(F)c(N3CCN(S(C)(=O)=O)CC34CC4)cc21] | 2,6-Difluoro-3-(5-fluoro-1-methyl-6-(7- (methylsulfonyl)-4,7-diazaspiro[2.5]octan-4-yl)- 1H-indazol-3-yl)-5-(trifluoromethyl)phenol |
| 9.118 | 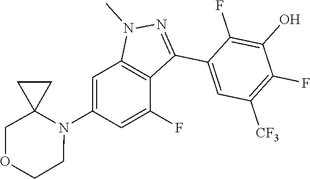 [SMILES: Cn1nc(-c2cc(C(F)(F)F)c(F)c(O)c2F)c2c(F)cc(N3CCOCC34CC4)cc21] | 2,6-Difluoro-3-(4-fluoro-1-methyl-6-(7-oxa-4- azaspiro[2.5]octan-4-yl)-1H-indazol-3-yl)-5- (trifluoromethyl)phenol |
| 9.119 | 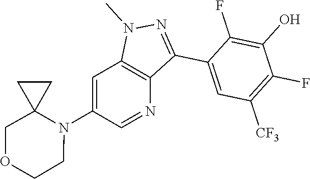 [SMILES: Cn1nc(-c2cc(C(F)(F)F)c(F)c(O)c2F)c2ncc(N3CCOCC34CC4)cc21] | 2,6-Difluoro-3-(1-methyl-6-(7-oxa-4- azaspiro[2.5]octan-4-yl)-1H-pyrazolo[4,3- b]pyridin-3-yl)-5-(trifluoromethyl)phenol |
| 9.120 | 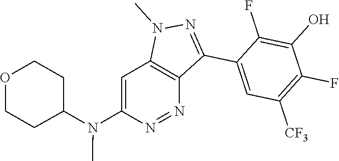 [SMILES: CN(c1cc2c(nn1)c(-c1cc(C(F)(F)F)c(F)c(O)c1F)nn2C)C1CCOCC1] | 2,6-Difluoro-3-(1-methyl-6-(methyl(tetrahydro- 2H-pyran-4-yl)amino)-1H-pyrazolo[4,3- c]pyridazin-3-yl)-5-(trifluoromethyl)phenol |
| 9.121 | 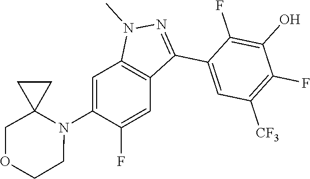 [SMILES: Cn1nc(-c2cc(C(F)(F)F)c(F)c(O)c2F)c2cc(F)c(N3CCOCC34CC4)cc21] | 2,6-Difluoro-3-(5-fluoro-1-methyl-6-(7-oxa-4- azaspiro[2.5]octan-4-yl)-1H-indazol-3-yl)-5- (trifluoromethyl)phenol |
| 9.122 | 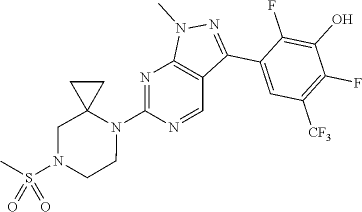 [SMILES: Cn1nc(-c2cc(C(F)(F)F)c(F)c(O)c2F)c2cnc(N3CCN(S(C)(=O)=O)CC34CC4)nc21] | 2,6-Difluoro-3-(1-methyl-6-(7- (methylsulfonyl)-4,7-diazaspiro[2.5]octan-4-yl)- 1H-pyrazolo[3,4-d]pyrimidin-3-yl)-5- (trifluoromethyl)phenol |
| 9.123 | 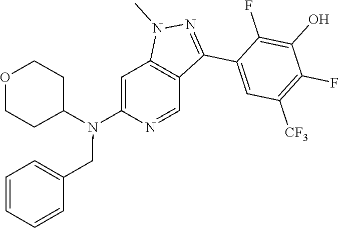 [SMILES: Cn1nc(-c2cc(C(F)(F)F)c(F)c(O)c2F)c2cnc(N(Cc3ccccc3)C3CCOCC3)cc21] | 3-(6-(Benzyl(tetrahydro-2H-pyran-4-yl)amino)- 1-methyl-1H-pyrazolo[4,3-c]pyridin-3-yl)-2,6- difluoro-5-(trifluoromethyl)phenol |
| 9.124 | 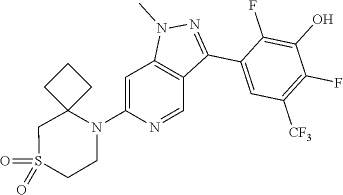 [SMILES: Cn1nc(-c2cc(C(F)(F)F)c(F)c(O)c2F)c2cnc(N3CCS(=O)(=O)CC34CCC4)cc21] | 5-(3-(2,4-Difluoro-3-hydroxy-5- (trifluoromethyl)phenyl)-1-methyl-1H- pyrazolo[4,3-c]pyridin-6-yl)-8-thia-5- azaspiro[3.5]nonane 8,8-dioxide |
| 9.125 | 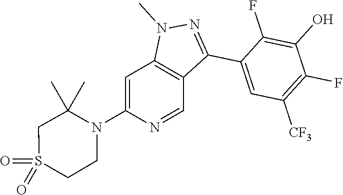 [SMILES: Cn1nc(-c2cc(C(F)(F)F)c(F)c(O)c2F)c2cnc(N3CCS(=O)(=O)CC3(C)C)cc21] | 4-(3-(2,4-Difluoro-3-hydroxy-5- (trifluoromethyl)phenyl)-1-methyl-1H- pyrazolo[4,3-c]pyridin-6-yl)-3,3- dimethylthiomorpholine 1,1-dioxide |
| 9.126 | 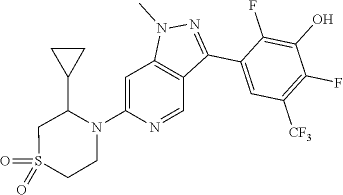 [SMILES: Cn1nc(-c2cc(C(F)(F)F)c(F)c(O)c2F)c2cnc(N3CCS(=O)(=O)CC3C3CC3)cc21] | 3-Cyclopropyl-4-(3-(2,4-difluoro-3-hydroxy-5- (trifluoromethyl)phenyl)-1-methyl-1H- pyrazolo[4,3-c]pyridin-6-yl)thiomorpholine 1,1- dioxide |
| 9.127 | 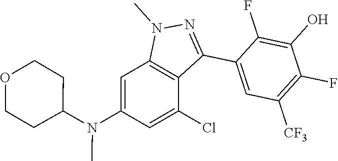 [SMILES: CN(c1cc(Cl)c2c(-c3cc(C(F)(F)F)c(F)c(O)c3F)nn(C)c2c1)C1CCOCC1] | 3-(4-Chloro-1-methyl-6-(methyl(tetrahydro-2H- pyran-4-yl)amino)-1H-indazol-3-yl)-2,6- difluoro-5-(trifluoromethyl)phenol |
| 9.128 | 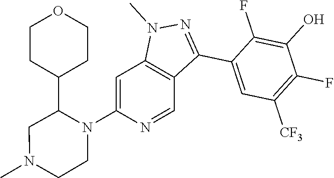 [SMILES: CN1CCN(c2cc3c(cn2)c(-c2cc(C(F)(F)F)c(F)c(O)c2F)nn3C)C(C2CCOCC2)C1] | 2,6-Difluroo-3-(1-methyl-6-(4-methyl-2- (tetrahydro-2H-pyran-4-yl)piperazin-1-yl)-1H- pyrazolo[4,3-c]pyridin-3-yl)-5- (trifluoromethyl)phenol |
| 9.129 | 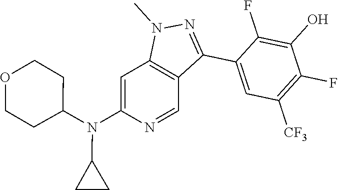 [SMILES: Cn1nc(-c2cc(C(F)(F)F)c(F)c(O)c2F)c2cnc(N(C3CCOCC3)C3CC3)cc21] | 3-(6-(Cyclopropyl(tetrahydro-2H-pyran-4- yl)amino)-1-methyl-1H-pyrazolo[4,3-c]pyridin- 3-yl)-2,6-difluoro-5-(trifluoromethyl)phenol |
| 9.130 | 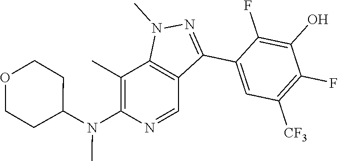 [SMILES: Cc1c(N(C)C2CCOCC2)ncc2c(-c3cc(C(F)(F)F)c(F)c(O)c3F)nn(C)c12] | 3-(1,7-Dimethyl-6-(methyl(tetrahydro-2H- pyran-4-yl)amino)-1H-pyrazolo[4,3-c]pyridin- 3-yl)-2,6-difluoro-5-(trifluoromethyl)phenol |
| 9.131 | 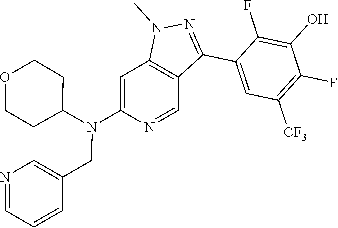 [SMILES: Cn1nc(-c2cc(C(F)(F)F)c(F)c(O)c2F)c2cnc(N(Cc3cccnc3)C3CCOCC3)cc21] | 2,6-Difluoro-3-(1-methyl-6-((pyridin-3- ylmethyl)(tetrahydro-2H-pyran-4-yl)amino)- 1H-pyrazolo[4,3-c]pyridin-3-yl)-5- (trifluoromethyl)phenol |
| 9.132 | 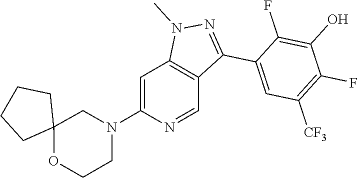 [SMILES: Cn1nc(-c2cc(C(F)(F)F)c(F)c(O)c2F)c2cnc(N3CCOC4(CCCC4)C3)cc21] | 2,6-Difluoro-3-(1-methyl-6-(6-oxa-9- azaspiro[4.5]decan-9-yl)-1H-pyrazolo[4,3- c]pyridin-3-yl)-5-(trifluoromethyl)phenol |
| 9-133 | 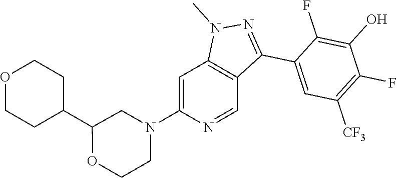 [SMILES: Cn1nc(-c2cc(C(F)(F)F)c(F)c(O)c2F)c2cnc(N3CCOC(C4CCOCC4)C3)cc21] | 2,6-Difluoro-3-(1-methyl-6-(2-(tetrahydro-2H- pyran-4-yl)morpholino)-1H-pyrazolo[4,3- c]pyridin-3-yl)-5-(trifluoromethyl)phenol |
| 9.134 | 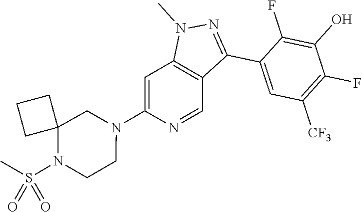 [SMILES: Cn1nc(-c2cc(C(F)(F)F)c(F)c(O)c2F)c2cnc(N3CCN(S(C)(=O)=O)C4(CCC4)C3)cc21] | 2,6-Difluoro-3-(1-methyl-6-(5- (methylsulfonyl)-5,8-diazaspiro[3.5]nonan-8- yl)-1H-pyrazolo[4,3-c]pyridin-3-yl)-5- (trifluoromethyl)phenol |
| 9.135 | 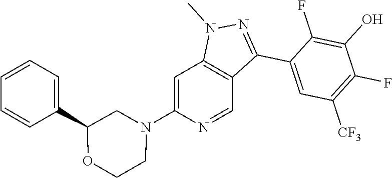 [SMILES: Cn1nc(-c2cc(C(F)(F)F)c(F)c(O)c2F)c2cnc(N3CCO[C@@H](c4ccccc4)C3)cc21] | (S)-2,6-Difluoro-3-(1-methyl-6-(2- phenylmorpholino)-1H-pyrazolo[4,3-c]pyridin- 3-yl)-5-(trifluoromethyl)phenol |
| 9.136 | 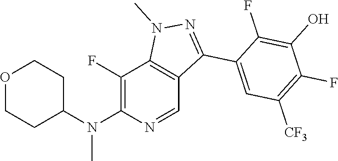 [SMILES: CN(c1ncc2c(-c3cc(C(F)(F)F)c(F)c(O)c3F)nn(C)c2c1F)C1CCOCC1] | 2,6-Difluoro-3-(7-fluoro-1-methyl-6- (methyl(tetrahydro-2H-pyran-4-yl)amino)-1H- pyrazolo[4,3-c]pyridin-3-yl)-5- (trifluoromethyl)phenol |
| 9.137 | 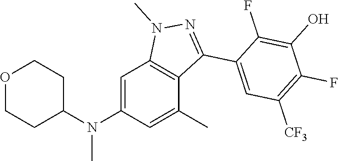 [SMILES: Cc1cc(N(C)C2CCOCC2)cc2c1c(-c1cc(C(F)(F)F)c(F)c(O)c1F)nn2C] | 3-(1,4-Dimethyl-6-(methyl(tetrahydro-2H- pyran-4-yl)amino)-1H-indazol-3-yl)-2,6- difluoro-5-(trifluoromethyl)phenol |
| 9.138 | 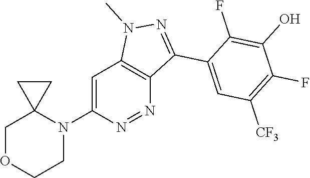 [SMILES: Cn1nc(-c2cc(C(F)(F)F)c(F)c(O)c2F)c2nnc(N3CCOCC34CC4)cc21] | 2,6-Difluoro-3-(1-methyl-6-(7-oxa-4- azaspiro[2.5]octan-4-yl)-1H-pyrazolo[4,3- c]pyridazin-3-yl)-5-(trifluoromethyl)phenol |
| 9.139 | 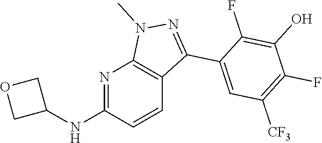 [SMILES: Cn1nc(-c2cc(C(F)(F)F)c(F)c(O)c2F)c2ccc(NC3COC3)nc21] | 2,6-Difluoro-3-(1-methyl-6-(oxetan-3-ylamino)- 1H-pyrazolo[3,4-b]pyridin-3-yl)-5- (trifluoromethyl)phenol |
| 9.140 | 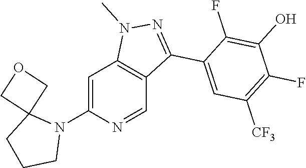 [SMILES: Cn1nc(-c2cc(C(F)(F)F)c(F)c(O)c2F)c2cnc(N3CCCC34COC4)cc21] | 2,6-Difluoro-3-(1-methyl-6-(2-oxa-5- azaspiro[3.4]octan-5-yl)-1H-pyrazolo[4,3- c]pyridin-3-yl)-5-(trifluoromethyl)phenol |
| 9.141 | 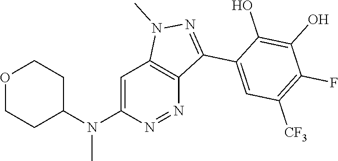 [SMILES: CN(c1cc2c(nn1)c(-c1cc(C(F)(F)F)c(F)c(O)c1O)nn2C)C1CCOCC1] | 3-Fluoro-6-(1-methyl-6-(methyl(tetrahydro-2H- pyran-4-yl)amino)-1H-pyrazolo[4,3- c]pyridazin-3-yl)-4-(trifluoromethyl)benzene- 1,2-diol |
| 10 | 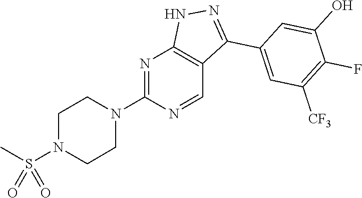 [SMILES: CS(=O)(=O)N1CCN(c2ncc3c(-c4cc(O)c(F)c(C(F)(F)F)c4)n[nH]c3n2)CC1] | 2-Fluoro-5-(6-(4-(methylsulfonyl)piperazin-1- yl)-1H-pyrazolo[3,4-d]pyrimidin-3-yl)-3- (trifluoromethyl)phenol |
| 10.01 | 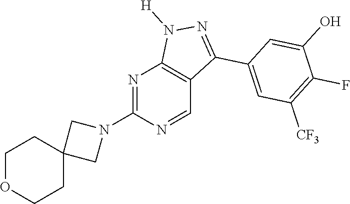 [SMILES: Oc1cc(-c2n[nH]c3nc(N4CC5(CCOCC5)C4)ncc23)cc(C(F)(F)F)c1F] | 5-(6-(7-Oxa-2-azaspiro[3.5]nonan-2-yl)-1H- pyrazolo[3,4-d]pyrimidin-3-yl)-2-fluoro-3- (trifluoromethyl)phenol |
| 10.02 | 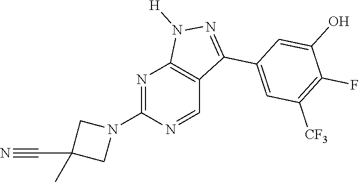 [SMILES: CC1(C#N)CN(c2ncc3c(-c4cc(O)c(F)c(C(F)(F)F)c4)n[nH]c3n2)C1] | 1-(3-(4-Fluoro-3-hydroxy-5- (trifluoromethyl)phenyl)-1H-pyrazolo[3,4- d]pyrimidin-6-yl)-3-methylazetidine-3- carbonitrile |
| 10.03 | 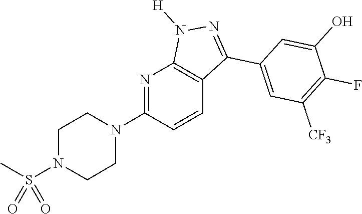 [SMILES: CS(=O)(=O)N1CCN(c2ccc3c(-c4cc(O)c(F)c(C(F)(F)F)c4)n[nH]c3n2)CC1] | 2-Fluoro-5-(6-(4-methylsulfonyl)piperazin-1- yl)-1H-pyrazolo[3,4-b]pyridin-3-yl)-3- (trifluoromethyl)phenol |
| 10.04 | 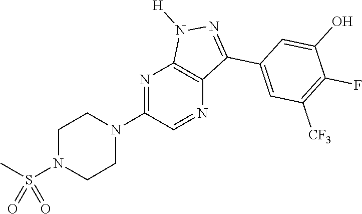 [SMILES: CS(=O)(=O)N1CCN(c2cnc3c(-c4cc(O)c(F)c(C(F)(F)F)c4)n[nH]c3n2)CC1] | 2-Fluoro-5-(6-(4-(methylsulfonyl)piperazin-1- yl)-1H-pyrazolo[3,4-b]pyrazin-3-yl)-3- ((trifluoromethyl)phenol |
| 10.05 | 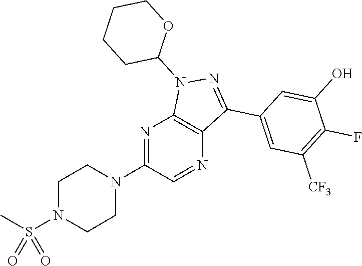 [SMILES: CS(=O)(=O)N1CCN(c2cnc3c(-c4cc(O)c(F)c(C(F)(F)F)c4)nn(C4CCCCO4)c3n2)CC1] | 2-Fluoro-5-(6-(4-(methylsulfonyl)piperazin-1- yl)-1-(tetrahydro-2H-pyran-2-yl)-1H- pyrazolo[3,4-b]pyrazin-3-yl)-3- (trifluoromethyl)phenol |
| 10.06 | 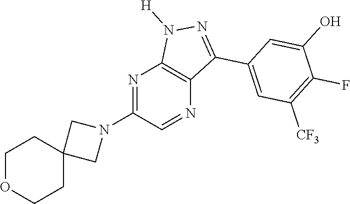 [SMILES: Oc1cc(-c2n[nH]c3nc(N4CC5(CCOCC5)C4)cnc23)cc(C(F)(F)F)c1F] | 5-(6-(7-Oxa-2-azapiro[3.5]nonan-2-yl)-1H- pyrazolo[3,4-b]pyrazin-3-yl)-2-fluoro-3- (trifluoromethyl)phenol |
| 11 | 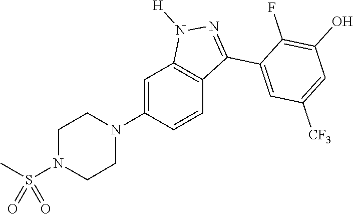 [SMILES: CS(=O)(=O)N1CCN(c2ccc3c(-c4cc(C(F)(F)F)cc(O)c4F)n[nH]c3c2)CC1] | 2-Fluoro-3-(6-(4-(methylsulfonyl)piperazin-1- yl)-1H-indazol-3-yl)-5-(trifluoromethyl)phenol |
| 11.01 | 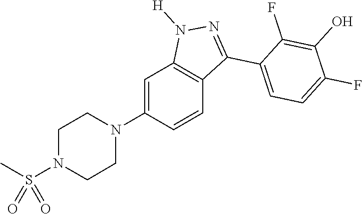 [SMILES: CS(=O)(=O)N1CCN(c2ccc3c(-c4ccc(F)c(O)c4F)n[nH]c3c2)CC1] | 2,6-Difluoro-3-(6-(4-(methylsulfonyl)piperazin- 1-yl)-1H-indazol-3-yl)phenol |
| 11.02 | 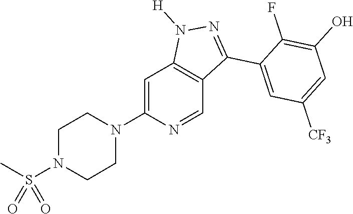 [SMILES: CS(=O)(=O)N1CCN(c2cc3[nH]nc(-c4cc(C(F)(F)F)cc(O)c4F)c3cn2)CC1] | 2-Fluoro-3-(6-(4-(methylsulfonyl)piperazin-1- yl)-1H-pyrazolo[4,3-c]pyridin-3-yl)-5- (trifluoromethyl)phenol |
| 11.03 | 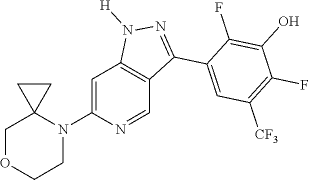 [SMILES: Oc1c(F)c(-c2n[nH]c3cc(N4CCOCC45CC5)ncc23)cc(C(F)(F)F)c1F] | 3-(6-(7-Oxa-4-azaspiro[2.5]octan-4-yl)-1H- pyrazolo[4,3-c]pyridin-3-yl)-2,6-difluoro-5- (trifluoromethyl)phenol |
| 11.04 | 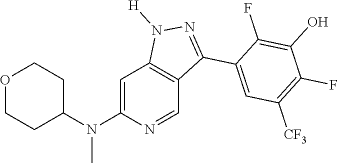 [SMILES: CN(c1cc2[nH]nc(-c3cc(C(F)(F)F)c(F)c(O)c3F)c2cn1)C1CCOCC1] | 2,6-Difluoro-3-(6-(methyl(tetrahydro-2H-pyran- 4-yl)amino)-1H-pyrazolo[4,3-c]pyridin-3-yl)-5- (trifluoromethyl)phenol |
| 12 | 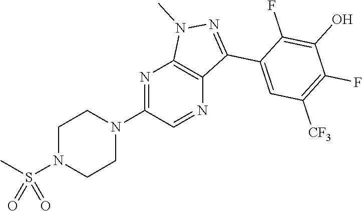 [SMILES: Cn1nc(-c2cc(C(F)(F)F)c(F)c(O)c2F)c2ncc(N3CCN(S(C)(=O)=O)CC3)nc21] | 2,6-Difluoro-3-(1-methyl-6-(4- (methylsulfonyl)piperazin-1-yl)-1H- pyrazolo[3,4-b]pyrazin-3-yl)-5- (trifluoromethyl)phenol |
| 12.01 | 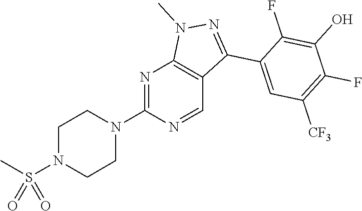 [SMILES: Cn1nc(-c2cc(C(F)(F)F)c(F)c(O)c2F)c2cnc(N3CCN(S(C)(=O)=O)CC3)nc21] | 2,6-Difluoro-3-(1-methyl-6-(4- (methylsulfonyl)piperazin-1-yl)-1H- pyrazolo[3,4-d]pyrimidin-3-yl)-5- (trifluoromethyl)phenol |
| 12.02 | 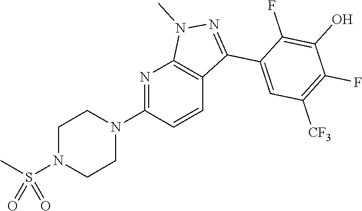 [SMILES: Cn1nc(-c2cc(C(F)(F)F)c(F)c(O)c2F)c2ccc(N3CCN(S(C)(=O)=O)CC3)nc21] | 2,6-Difluoro-3-(1-methyl-6-(4- (methylsulfonyl)piperazin-1-yl)-1H- pyrazolo[3,4-b]pyridin-3-yl)-5- (trifluoromethyl)phenol |
| 12.03 | 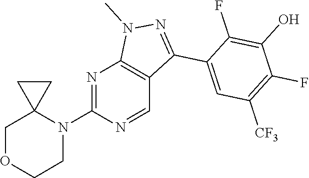 [SMILES: Cn1nc(-c2cc(C(F)(F)F)c(F)c(O)c2F)c2cnc(N3CCOCC34CC4)nc21] | 2,6-Difluoro-3-(1-methyl-6-(7-oxa-4- azaspiro[2.5]octan-4-yl)-1H-pyrazolo[3,4- d]pyrimidin-3-yl)-5-(trifluoromethyl)phenol |
| 12.04 | 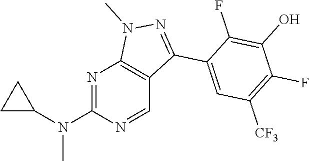 [SMILES: CN(c1ncc2c(-c3cc(C(F)(F)F)c(F)c(O)c3F)nn(C)c2n1)C1CC1] | 3-(6-(Cyclopropyl(methyl)amino)-1-methyl-1H- pyrazolo[3,4-d]pyrimidin-3-yl)-2,6-difluoro-5- (trifluoromethyl)phenol |
| 12.05 | 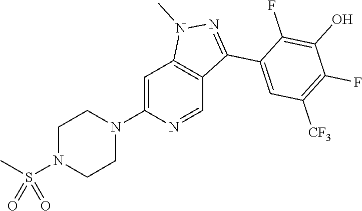 [SMILES: Cn1nc(-c2cc(C(F)(F)F)c(F)c(O)c2F)c2cnc(N3CCN(S(C)(=O)=O)CC3)cc21] | 2,6-Difluoro-3-(1-methyl-6-(4- (methylsulfonyl)piperazin-1-yl)-1H- pyrazolo[4,3-c]pyridin-3-yl)-5- (trifluoromethyl)phenol |
| 12.06 | 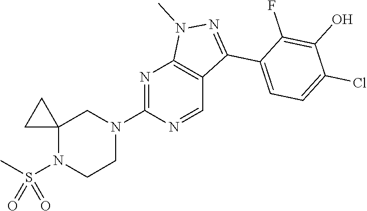 [SMILES: Cn1nc(-c2ccc(Cl)c(O)c2F)c2cnc(N3CCN(S(C)(=O)=O)C4(CC4)C3)nc21] | 6-Chloro-2-fluoro-3-(1-methyl-6-(4- (methylsulfonyl)-4,7-diazaspiro[2.5]octan-7-yl)- 1H-pyrazolo[3,4-d]pyrimidin-3-yl)phenol |
| 12.07 | 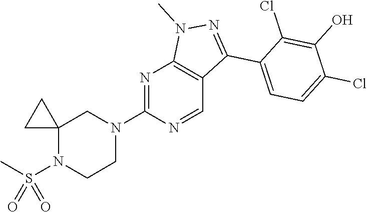 [SMILES: Cn1nc(-c2ccc(Cl)c(O)c2Cl)c2cnc(N3CCN(S(C)(=O)=O)C4(CC4)C3)nc21] | 2,6-Dichloro-3-(1-methyl-6-(4- (methylsulfonyl)-4,7-diazaspiro[2.5]octan-7-yl)- 1H-pyrazolo[3,4-d]pyrimidin-3-yl)phenol |
| 12.08 | 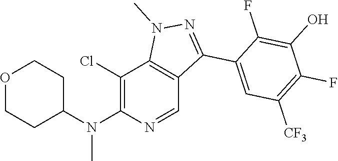 [SMILES: CN(c1ncc2c(-c3cc(C(F)(F)F)c(F)c(O)c3F)nn(C)c2c1Cl)C1CCOCC1] | 3-(7-Chloro-1-methyl-6-(methyl(tetrahydro-2H- pyran-4-yl)amino)-1H-pyrazolo[4,3-c]pyridin- 3-yl)-2,6-difluoro-5-(trifluoromethyl)phenol |
| 12.09 | 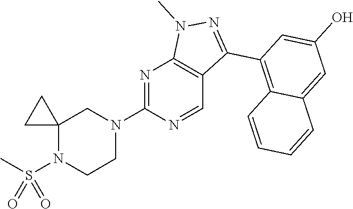 [SMILES: Cn1nc(-c2cc(O)cc3ccccc23)c2cnc(N3CCN(S(C)(=O)=O)C4(CC4)C3)nc21] | 4-(1-Methyl-6-(4-(methylsulfonyl)-4,7- diazaspiro[2.5]octan-7-yl)-1H-pyrazolo[3,4- d]pyrimidin-3-yl)naphthalen-2-ol |
| 12.10 | 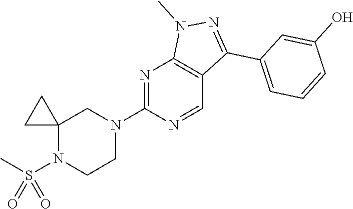 [SMILES: Cn1nc(-c2cccc(O)c2)c2cnc(N3CCN(S(C)(=O)=O)C4(CC4)C3)nc21] | 3-(1-Methyl-6-(4-(methylsulfonyl)-4,7- diazaspiro[2.5]octan-7-yl)-1H-pyrazolo[3,4- d]pyrimidin-3-yl)phenol |
| 13 | 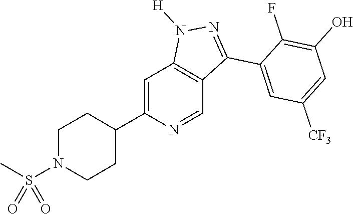 [SMILES: CS(=O)(=O)N1CCC(c2cc3[nH]nc(-c4cc(C(F)(F)F)cc(O)c4F)c3cn2)CC1] | 2-Fluoro-3-(6-(1-(methylsulfonyl)piperidin-4- yl)-1H-pyrazolo[4,3-c]pyridin-3-yl)-5- (trifluoromethyl)phenol |
| 13.01 | 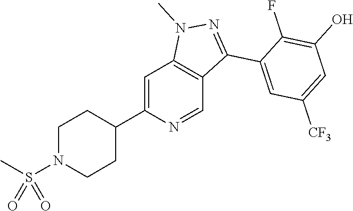 [SMILES: Cn1nc(-c2cc(C(F)(F)F)cc(O)c2F)c2cnc(C3CCN(S(C)(=O)=O)CC3)cc21] | 2-Fluoro-3-(1-methyl-6-(1- (methylsulfonyl)piperidin-4-yl)-1H- pyrazolo[4,3-c]pyridin-3-yl)-5- (trifluoromethyl)phenol |
| 14 | 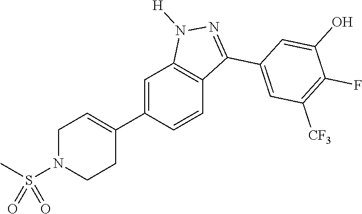 [SMILES: CS(=O)(=O)N1CC=C(c2ccc3c(-c4cc(O)c(F)c(C(F)(F)F)c4)n[nH]c3c2)CC1] | 2-Fluoro-5-(6-(1-(methyslulfonyl)-1,2,3,6- tetrahydropyridin-4-yl)-1H-indazol-3-yl)-3- (trifluoromethyl)phenol |
| 14.01 | 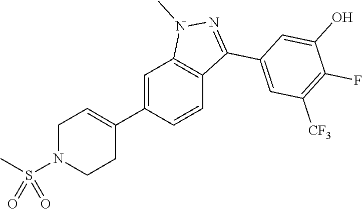 [SMILES: Cn1nc(-c2cc(O)c(F)c(C(F)(F)F)c2)c2ccc(C3=CCN(S(C)(=O)=O)CC3)cc21] | 2-Fluoro-5-(1-methyl-6-(1-(methylsulfonyl)- 1,2,3,6-tetrahydropyridin-4-yl)-1H-indazol-3- yl)-3-(trifluoromethyl)phenol |
| 14.02 | 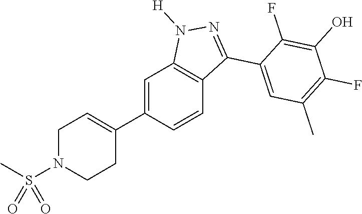 [SMILES: Cc1cc(-c2n[nH]c3cc(C4=CCN(S(C)(=O)=O)CC4)ccc23)c(F)c(O)c1F] | 2,6-Difluoro-3-methyl-5-(6-(1- (methylsulfonyl)-1,2,3,6-tetrahydropyridin-4- yl)-1H-indazol-3-yl-phenol |
| 14.03 | 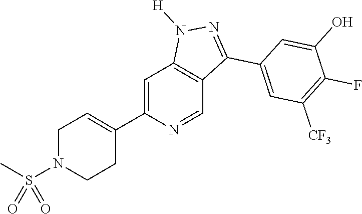 [SMILES: CS(=O)(=O)N1CC=C(c2cc3[nH]nc(-c4cc(O)c(F)c(C(F)(F)F)c4)c3cn2)CC1] | 2-Fluoro-5-(6-(1-(methylsulfonyl)-1,2,3,6- tetrahydropyridin-4-yl)-1H-pyrazolo[4,3- c]pyridin-3-yl)-3-(trifluoromethyl)phenol |
| 15 | 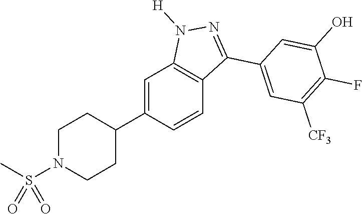 [SMILES: CS(=O)(=O)N1CCC(c2ccc3c(-c4cc(O)c(F)c(C(F)(F)F)c4)n[nH]c3c2)CC1] | 2-Fluoro-5-(6-(1-(methylsulfonyl)piperidin-4- yl)-1H-indazolo-3-yl)-3-(trifluoromethyl)phenol |
| 15.01 | 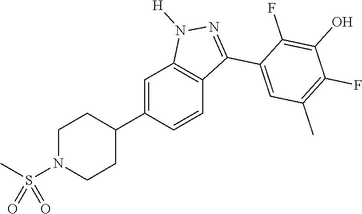 [SMILES: Cc1cc(-c2n[nH]c3cc(C4CCN(S(C)(=O)=O)CC4)ccc23)c(F)c(O)c1F] | 2,6-Difluoro-3-methyl-5-(6-(1- (methylsulfonyl)piperidin-4-yl)-1H-indazol-3- yl)phenol |
| 15.02 | 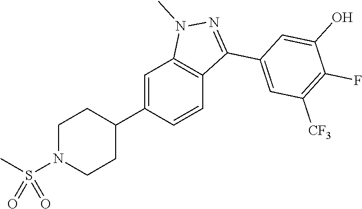 [SMILES: Cn1nc(-c2cc(O)c(F)c(C(F)(F)F)c2)c2ccc(C3CCN(S(C)(=O)=O)CC3)cc21] | 2-Fluoro-5-(1-methyl-6-(1- (methylsulfonyl)piperidin-4-yl)-1H-indazol-3- yl)-3-(trifluoromethyl)phenol |
| 15.03 | 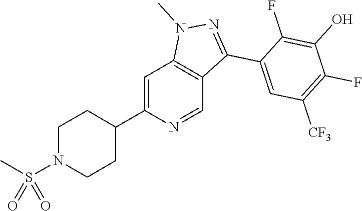 [SMILES: Cn1nc(-c2cc(C(F)(F)F)c(F)c(O)c2F)c2cnc(C3CCN(S(C)(=O)=O)CC3)cc21] | 2,6-Difluoro-3-(1-methyl-6-(1- (methylsulfonyl)piperidin-4-yl)-1H- pyrazolo[4,3-c]pyridin-3-yl)-5- (trifluoromethyl)phenol |
| 15.04 | 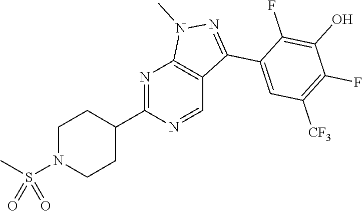 [SMILES: Cn1nc(-c2cc(C(F)(F)F)c(F)c(O)c2F)c2cnc(C3CCN(S(C)(=O)=O)CC3)nc21] | 2,6-Difluoro-3-(1-methyl-6-(1- (methylsulfonyl)piperidin-4-yl)-1H- pyrazolo[3,4-d]pyrimidin-3-yl)-5- (trifluoromethyl)phenol |
| 16 | 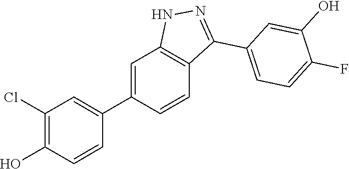 [SMILES: Oc1cc(-c2n[nH]c3cc(-c4ccc(O)c(Cl)c4)ccc23)ccc1F] | 2-Chloro-4-(3-(4-fluoro-3-hydroxyphenyl)-1H- indazol-6-yl)phenol |
| 17 | 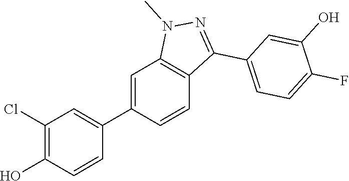 [SMILES: Cn1nc(-c2ccc(F)c(O)c2)c2ccc(-c3ccc(O)c(Cl)c3)cc21] | 2-Chloro-4-(3-(4-fluoro-3-hydroxyphenyl)-1- methyl-1H-indazol-6-yl)phenol |
| 18 | 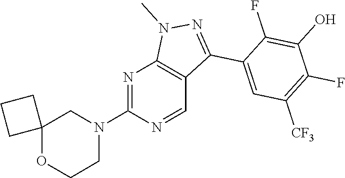 [SMILES: Cn1nc(-c2cc(C(F)(F)F)c(F)c(O)c2F)c2cnc(N3CCOC4(CCC4)C3)nc21] | 2,6-Difluoro-3-(1-methyl-6-(5-oxa-8- azaspiro[3.5]nonan-8-yl)-1H-pyrazolo[3,4- d]pyrimidin-3-yl)-5-(trifluoromethyl)phenol |
| 18.01 | 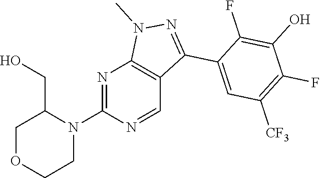 [SMILES: Cn1nc(-c2cc(C(F)(F)F)c(F)c(O)c2F)c2cnc(N3CCOCC3CO)nc21] | 2,6-Difluoro-3-(6-(3- (hydroxymethyl)morpholino)-1-methyl-1H- pyrazolo[3,4-d]pyrimidin-3-yl)-5- (trifluoromethyl)phenol |
| 18.02 | 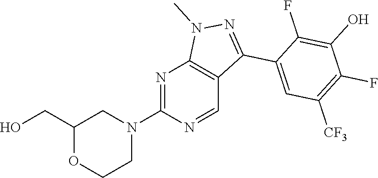 [SMILES: Cn1nc(-c2cc(C(F)(F)F)c(F)c(O)c2F)c2cnc(N3CCOC(CO)C3)nc21] | 2,6-Difluoro-3-(6-(2- (hydroxymethyl)morpholino)-1-methyl-1H- pyrazolo[3,4-d]pyrimidin-3-yl)-5- (trifluoromethyl)phenol |
| 18.03 | 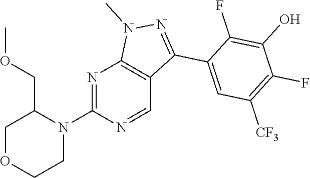 [SMILES: COCC1COCCN1c1ncc2c(-c3cc(C(F)(F)F)c(F)c(O)c3F)nn(C)c2n1] | 2,6-Difluoro-3-(6-(3- (methoxymethyl)morpholino)-1-methyl-1H- pyrazolo[3,4-d]pyrimidin-3-yl)-5- (trifluoromethyl)phenol |
| 18.04 | 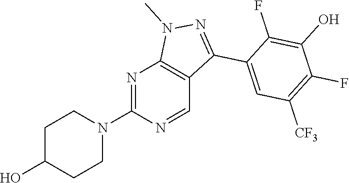 [SMILES: Cn1nc(-c2cc(C(F)(F)F)c(F)c(O)c2F)c2cnc(N3CCC(O)CC3)nc21] | 1-(3-(2,4-Difluoro-3-hydroxy-5- (trifluoromethyl)phenyl)-1-methyl-1H- pyrazolo[3,4-d]pyrimidin-6-yl)piperidin-4-ol |
| 18.05 | 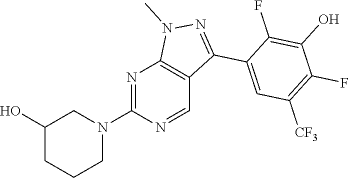 [SMILES: Cn1nc(-c2cc(C(F)(F)F)c(F)c(O)c2F)c2cnc(N3CCCC(O)C3)nc21] | 1-(3-(2,4-Difluoro-3-hydroxy-5- (trifluoromethyl)phenyl)-1-methyl-1H- pyrazolo[3,4-d]pyrimidin-6-yl)piperidin-3-ol |
| 18.06 | 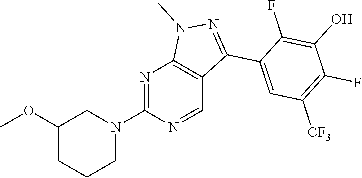 [SMILES: COC1CCCN(c2ncc3c(-c4cc(C(F)(F)F)c(F)c(O)c4F)nn(C)c3n2)C1] | 2,6-Difluoro-3-(6-(3-methoxypiperidin-1-yl)-1- methyl-1H-pyrazolo[3,4-d]pyrimidin-3-yl)-5- (trifluoromethyl)phenol |
| 18.07 | 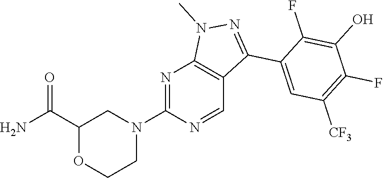 [SMILES: Cn1nc(-c2cc(C(F)(F)F)c(F)c(O)c2F)c2cnc(N3CCOC(C(N)=O)C3)nc21] | 4-(3-(2,4-Difluoro-3-hydroxy-5- (trifluoromethyl)phenyl)-1-methyl-1H- pyrazolo[3,4-d]pyrimidin-6-yl)morpholine-2- carboxamide |
| 18.08 | 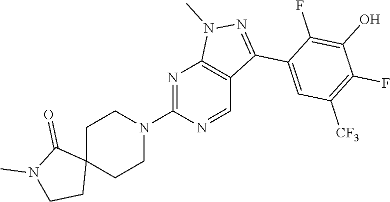 [SMILES: CN1CCC2(CCN(c3ncc4c(-c5cc(C(F)(F)F)c(F)c(O)c5F)nn(C)c4n3)CC2)C1=O] | 8-(3-(2,4-Difluoro-3-hydroxy-5- (trifluoromethyl)phenyl)-1-methyl-1H- pyrazolo[3,4-d]pyrimidin-6-yl)-2-methyl-2,8- diazaspiro[4.5]decan-1-one |
| 18.09 | 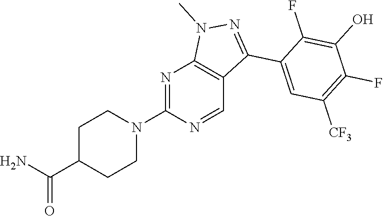 [SMILES: Cn1nc(-c2cc(C(F)(F)F)c(F)c(O)c2F)c2cnc(N3CCC(C(N)=O)CC3)nc21] | 1-(3-(2,4-Difluoro-3-hydroxy-5- (trifluoromethyl)phenyl)-1-methyl-1H- pyrazolo[3,4-d]pyrimidin-6-yl)piperidine-4- carboxamide |
| 18.10 | 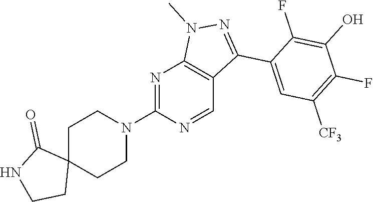 [SMILES: Cn1nc(-c2cc(C(F)(F)F)c(F)c(O)c2F)c2cnc(N3CCC4(CCNC4=O)CC3)nc21] | 8-(3-(2,4-difluoro-3-hydroxy-5- (trifluoromethyl)phenyl)-1-methyl-1H- pyrazolo[3,4-d]pyrimidin-6-yl)-2,8- diazaspiro[4.5]decan-1-one |
| 18.11 | 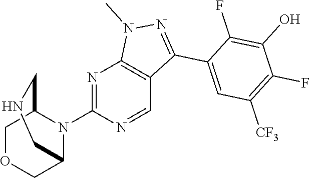 [SMILES: Cn1nc(-c2cc(C(F)(F)F)c(F)c(O)c2F)c2cnc(N3[C@@H]4CNC[C@H]3COC4)nc21] | 3-(6-((1R,54S)-3-oxa-7,9- diazabicyclo[3.3.1]nonan-9-yl)-1-methyl-1H- pyrazolo[3,4-d]pyrimidin-3-yl)-2,6-difluoro-5- (trifluoromethyl)phenol |
| 18.12 | 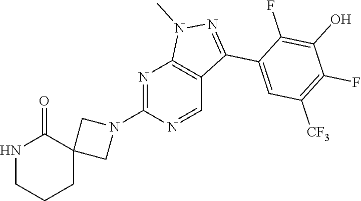 [SMILES: Cn1nc(-c2cc(C(F)(F)F)c(F)c(O)c2F)c2cnc(N3CC4(CCCNC4=O)C3)nc21] | 2-(3-(2,4-Difluoro-3-hydroxy-5- (trifluoromethyl)phenyl)-1-methyl-1H- pyrazolo[3,4-d]pyrimidin-6-yl)-2,6- diazaspiro[3.5]nonan-5-one |
| 18.13 | 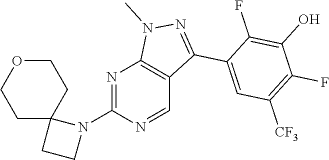 [SMILES: Cn1nc(-c2cc(C(F)(F)F)c(F)c(O)c2F)c2cnc(N3CCC34CCOCC4)nc21] | 2,6-Difluoro-3-(1-methyl-6-(7-oxa-1- azaspiro[3.5]nonan-1-yl)-1H-pyrazolo[3,4- d]pyrimidin-3-yl)-5-(trifluoromethyl)phenol |
| 18.14 | 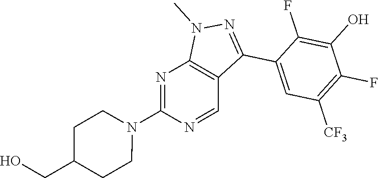 [SMILES: Cn1nc(-c2cc(C(F)(F)F)c(F)c(O)c2F)c2cnc(N3CCC(CO)CC3)nc21] | 2,6-Difluoro-3-(6-(4-(hydroxymethyl)piperidin- 1-yl)-1-methyl-1H-pyrazolo[3,4-d]pyrimidin-3- yl)-5-(trifluoromethyl)phenol |
| 18.15 | 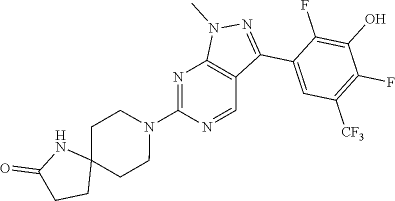 [SMILES: Cn1nc(-c2cc(C(F)(F)F)c(F)c(O)c2F)c2cnc(N3CCC4(CCC(=O)N4)CC3)nc21] | 8-(3-(2,4-Difluoro-3-hydroxy-5- (trifluoromethyl)phenyl)-1-methyl-1H- pyrazolo[3,4-d]pyrimidin-6-yl)-1,8- diazaspiro[4.5]decan-2-one |
| 18.16 | 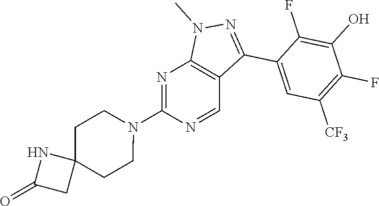 [SMILES: Cn1nc(-c2cc(C(F)(F)F)c(F)c(O)c2F)c2cnc(N3CCC4(CC3)CC(=O)N4)nc21] | 7-(3-(2,4-Difluoro-3-hydroxy-5- (trifluoromethyl)phenyl)-1-methyl-1H- pyrazolo[3,4-d]pyrimidin-6-yl)-1,7- diazaspiro[3.5]nonan-2-one |
| 18.17 | 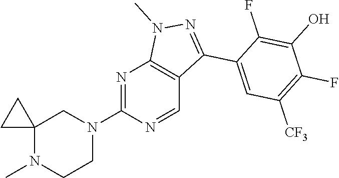 [SMILES: CN1CCN(c2ncc3c(-c4cc(C(F)(F)F)c(F)c(O)c4F)nn(C)c3n2)CC12CC2] | 2,6-Difluoro-3-(1-methyl-6-(4-methyl-4,7- diazaspiro[2.5]octan-7-yl)-1H-pyrazolo[3,4- d]pyrimidin-3-yl)-5-(trifluoromethyl)phenol |
| 18.18 | 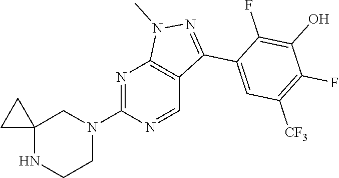 [SMILES: Cn1nc(-c2cc(C(F)(F)F)c(F)c(O)c2F)c2cnc(N3CCNC4(CC4)C3)nc21] | 2,6-Difluoro-3-(1-methyl-6-(4,7- diazaspiro[2.5]octan-7-yl)-1H-pyrazolo[3,4- d]pyrimidin-3-yl)-5-(trifluoromethyl)phenol |
| 18.19 | 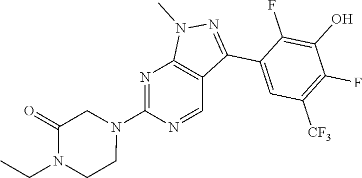 [SMILES: CCN1CCN(c2ncc3c(-c4cc(C(F)(F)F)c(F)c(O)c4F)nn(C)c3n2)CC1=O] | 4-(3-(2,4-Difluoro-3-hydroxy-5- (trifluoromethyl)phenyl)-1-methyl-1H- pyrazolo[3,4-d]pyrimidin-6-yl)-1- ethylpiperazin-2-one |
| 18.20 | 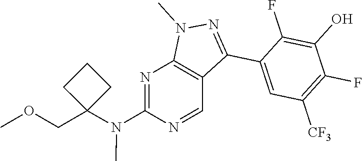 [SMILES: COCC1(N(C)c2ncc3c(-c4cc(C(F)(F)F)c(F)c(O)c4F)nn(C)c3n2)CCC1] | 2,6-Difluoro-3-(6-((1- (methoxymethyl)cyclobutyl)(methyl)amino)-1- methyl-1H-pyrazolo[3,4-d]pyrimidin-3-yl)-5- (trifluoromethyl)phenol |
| 18.21 | 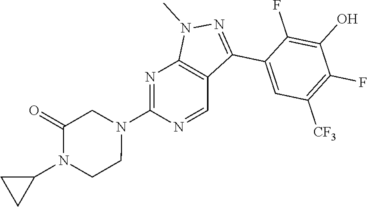 [SMILES: Cn1nc(-c2cc(C(F)(F)F)c(F)c(O)c2F)c2cnc(N3CCN(C4CC4)C(=O)C3)nc21] | 1-Cyclopropyl-4-(3-(2,4-difluoro-3-hydroxy-5- (trifluoromethyl)phenyl)-1-methyl-1H- pyrazolo[3,4-d]pyrimidin-6-yl)piperazin-2-one |
| 18.22 | 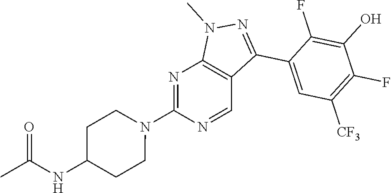 [SMILES: CC(=O)NC1CCN(c2ncc3c(-c4cc(C(F)(F)F)c(F)c(O)c4F)nn(C)c3n2)CC1] | N-(1-(3-(2,4-Difluoro-3-hydroxy-5- (trifluoromethyl)phenyl)-1-methyl-1H- pyrazolo[3,4-d]pyrimidin-6-yl)piperidin-4- yl)acetamide |
| 18.23 | 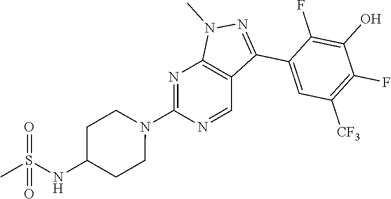 [SMILES: Cn1nc(-c2cc(C(F)(F)F)c(F)c(O)c2F)c2cnc(N3CCC(NS(C)(=O)=O)CC3)nc21] | N-(1-(3-(2,4-Difluoro-3-hydroxy-5- (trifluoromethyl)phenyl)-1-methyl-1H- pyrazolo[3,4-d]pyrimidin-6-yl)piperidin-4- yl)methanesulfonamide |
| 18.24 | 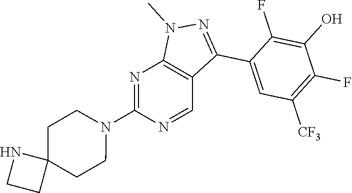 [SMILES: Cn1nc(-c2cc(C(F)(F)F)c(F)c(O)c2F)c2cnc(N3CCC4(CCN4)CC3)nc21] | 2,6-Difluoro-3-(1-methyl-6-(1,7- diazaspiro[3.5]nonan-7-yl)-1H-pyrazolo[3,4- d]pyrimidin-3-yl)-5-(trifluoromethyl)phenol |
| 18.25 | 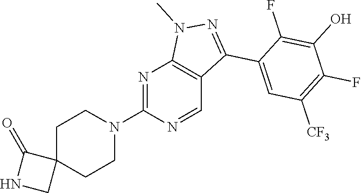 [SMILES: Cn1nc(-c2cc(C(F)(F)F)c(F)c(O)c2F)c2cnc(N3CCC4(CC3)CNC4=O)nc21] | 7-(3-(2,4-Difluoro-3-hydroxy-5- (trifluoromethyl)phenyl)-1-methyl-1H- pyrazolo[3,4-d]pyrimidin-6-yl)-2,7- diazaspiro[3.5]nonan-1-one |
| 18.26 | 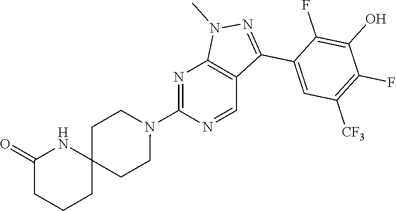 [SMILES: Cn1nc(-c2cc(C(F)(F)F)c(F)c(O)c2F)c2cnc(N3CCC4(CCCC(=O)N4)CC3)nc21] | 9-(3-(2,4-Difluoro-3-hydroxy-5- (trifluoromethyl)phenyl)-1-methyl-1H- pyrazolo[3,4-d]pyrimidin-6-yl)-1,9- diazaspiro[5.5]undecan-2-one |
| 18.27 | 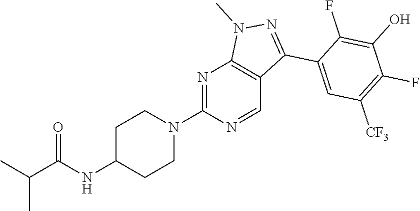 [SMILES: CC(C)C(=O)NC1CCN(c2ncc3c(-c4cc(C(F)(F)F)c(F)c(O)c4F)nn(C)c3n2)CC1] | N-(1-(3-(2,4-Difluoro-3-hydroxy-5- (trifluoromethyl)phenyl)-1-methyl-1H- pyrazolo[3,4-d]pyrimidin-6-yl)piperidin-4- yl)isobutyramide |
| 18.28 | 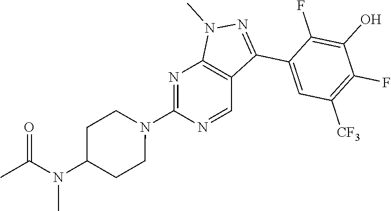 [SMILES: CC(=O)N(C)C1CCN(c2ncc3c(-c4cc(C(F)(F)F)c(F)c(O)c4F)nn(C)c3n2)CC1] | N-(1-(3-(2,4-Difluoro-3-hydroxy-5- (trifluoromethyl)phenyl)-1-methyl-1H- pyrazolo[3,4-d]pyrimidin-6-yl)piperidin-4-yl)- N-methylacetamide |
| 18.29 | 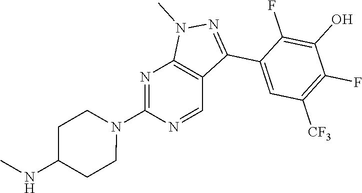 [SMILES: CNC1CCN(c2ncc3c(-c4cc(C(F)(F)F)c(F)c(O)c4F)nn(C)c3n2)CC1] | 2,6-Difluoro-3-(1-methyl-6-(4- (methylamino)piperidin-1-yl)-1H-pyrazolo[3,4- d]pyrimidin-3-yl)-5-(trifluoromethyl)phenol |
| 18.30 | 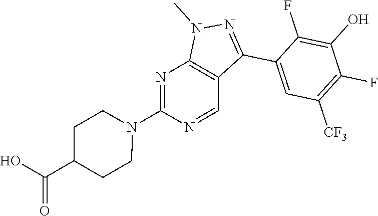 [SMILES: Cn1nc(-c2cc(C(F)(F)F)c(F)c(O)c2F)c2cnc(N3CCC(C(=O)O)CC3)nc21] | 1-(3-(2,4-Difluoro-3-hydroxy-5- (trifluoromethyl)phenyl)-1-methyl-1H- pyrazolo[3,4-d]pyrimidin-6-yl)piperidine-4- carboxylic Acid |
| 18.31 | 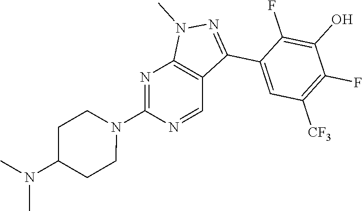 [SMILES: CN(C)C1CCN(c2ncc3c(-c4cc(C(F)(F)F)c(F)c(O)c4F)nn(C)c3n2)CC1] | 3-(6-(4-(Dimethylamino)piperidin-1-yl)-1- methyl-1H-pyrazolo[3,4-d]pyrimidin-3-yl)-2,6- difluoro-5-(trifluoromethyl)phenol |
| 18.32 | 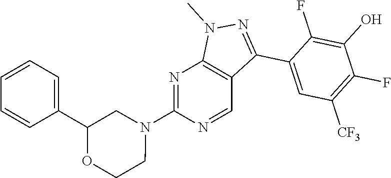 [SMILES: Cn1nc(-c2cc(C(F)(F)F)c(F)c(O)c2F)c2cnc(N3CCOC(c4ccccc4)C3)nc21] | 2,6-Difluoro-3-(1-methyl-6-(2- phenylmorpholino)-1H-pyrazolo[3,4- d]pyrimidin-3-yl)-5-(trifluoromethyl)phenol |
| 18.33 | 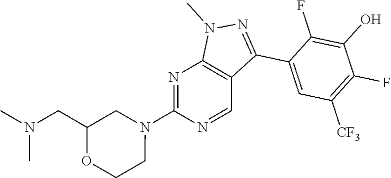 [SMILES: CN(C)CC1CN(c2ncc3c(-c4cc(C(F)(F)F)c(F)c(O)c4F)nn(C)c3n2)CCO1] | 3-(6-(2-((Dimethylamino)methyl)morpholino)- 1-methyl-1H-pyrazolo[3,4-d]pyrimidin-3-yl)- 2,6-difluoro-5-(trifluoromethyl)phenol |
| 18.34 | 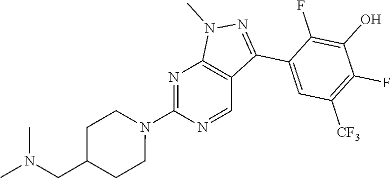 [SMILES: CN(C)CC1CCN(c2ncc3c(-c4cc(C(F)(F)F)c(F)c(O)c4F)nn(C)c3n2)CC1] | 3-(6-(4-((Dimethylamino)methyl)piperidin-1- yl)-1-methyl-1H-pyrazolo[3,4-d]pyrimidin-3- yl)-2,6-difluoro-5-(trifluoromethyl)phenol |
| 18.35 | 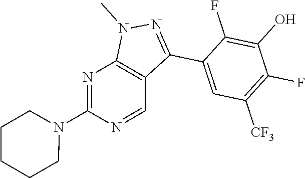 [SMILES: Cn1nc(-c2cc(C(F)(F)F)c(F)c(O)c2F)c2cnc(N3CCCCC3)nc21] | 2,6-Difluoro-3-(1-methyl-6-(piperidin-1-yl)-1H- pyrazolo[3,4-d]pyrimidin-3-yl)-5- (trifluoromethyl)phenol |
| 18.36 | 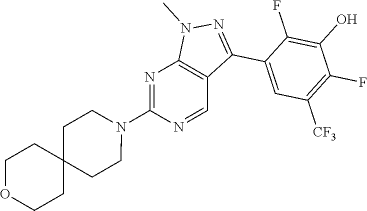 [SMILES: Cn1nc(-c2cc(C(F)(F)F)c(F)c(O)c2F)c2cnc(N3CCC4(CCOCC4)CC3)nc21] | 2,6-Difluoro-3-(1-methyl-6-(3-oxa-9- azaspiro[5.5]undecan-9-yl)-1H-pyrazolo[3,4- d]pyrimidin-3-yl)-5-(trifluoromethyl)phenol |
| 18.37 | 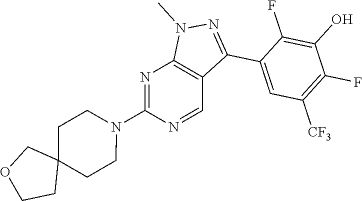 [SMILES: Cn1nc(-c2cc(C(F)(F)F)c(F)c(O)c2F)c2cnc(N3CCC4(CCOC4)CC3)nc21] | 2,6-Difluoro-3-(1-methyl-6-(2-oxa-8- azaspiro[4.5]decan-8-yl)-1H-pyrazolo[3,4- d]pyrimidin-3-yl)-5-(trifluoromethyl)phenol |
| 18.38 | 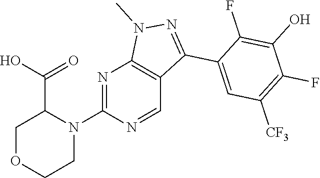 [SMILES: Cn1nc(-c2cc(C(F)(F)F)c(F)c(O)c2F)c2cnc(N3CCOCC3C(=O)O)nc21] | 4-(3-(2,4-Difluoro-3-hydroxy-5- (trifluoromethyl)phenyl)-1-methyl-1H- pyrazolo[3,4-d]pyrimidin-6-yl)morpholine-3- carboxylic Acid |
| 18.39 | 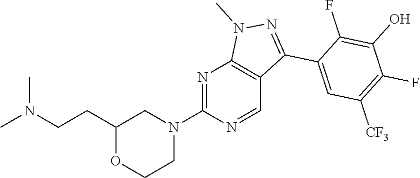 [SMILES: CN(C)CCC1CN(c2ncc3c(-c4cc(C(F)(F)F)c(F)c(O)c4F)nn(C)c3n2)CCO1] | 3-(6-(2-(2-(Dimethylamino)ethyl)morpholino)- 1-methyl-1H-pyrazolo[3,4-d]pyrimidin-3-yl)- 2,6-difluoro-5-(trifluoromethyl)phenol |
| 18.40 | 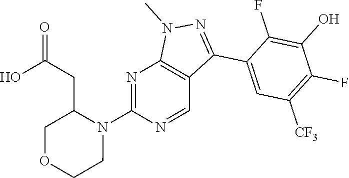 [SMILES: Cn1nc(-c2cc(C(F)(F)F)c(F)c(O)c2F)c2cnc(N3CCOCC3CC(=O)O)nc21] | 2-(4-(3-(2,4-Difluoro-3-hydroxy-5- (trifluoromethyl)phenyl)-1-methyl-1H- pyrazolo[3,4-d]pyrimidin-6-yl)morpholin-3- yl)acetic Acid |
| 18.41 | 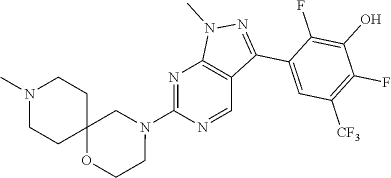 [SMILES: CN1CCC2(CC1)CN(c1ncc3c(-c4cc(C(F)(F)F)c(F)c(O)c4F)nn(C)c3n1)CCO2] | 2,6-Difluoro-3-(1-methyl-6-(9-methyl-1-oxa- 4,9-diazaspiro[5.5]undecan-4-yl)-1H- pyrazolo[3,4-d]pyrimidin-3-yl)-5- (trifluoromethyl)phenol |
| 18.42 | 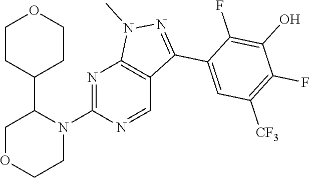 [SMILES: Cn1nc(-c2cc(C(F)(F)F)c(F)c(O)c2F)c2cnc(N3CCOCC3C3CCOCC3)nc21] | 2,6-Difluoro-3-(1-methyl-6-(3-(tetrahydro-2H- pyran-4-yl)morpholino)-1H-pyrazolo[3,4- d]pyrimidin-3-yl)-5-(trifluoromethyl)phenol |
| 18.43 | 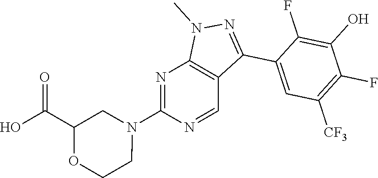 [SMILES: Cn1nc(-c2cc(C(F)(F)F)c(F)c(O)c2F)c2cnc(N3CCOC(C(=O)O)C3)nc21] | 4-(3-(2,4-Difluoro-3-hydroxy-5- (trifluoromethyl)phenyl)-1-methyl-1H- pyrazolo[3,4-d]pyrimidin-6-yl)morpholine-2- carboxylic Acid |
| 18.44 | 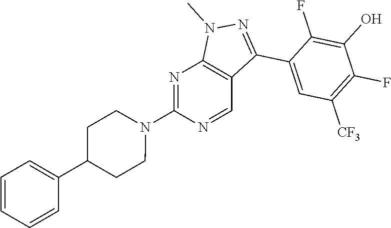 [SMILES: Cn1nc(-c2cc(C(F)(F)F)c(F)c(O)c2F)c2cnc(N3CCC(c4ccccc4)CC3)nc21] | 2,6-Difluoro-3-(1-methyl-6-(4-phenylpiperidin- 1-yl)-1H-pyrazolo[3,4-d]pyrimidin-3-yl)-5- (trifluoromethyl)phenol |
| 18.45 | 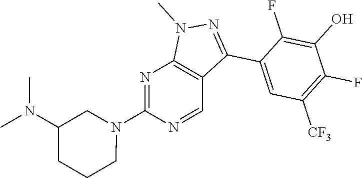 [SMILES: CN(C)C1CCCN(c2ncc3c(-c4cc(C(F)(F)F)c(F)c(O)c4F)nn(C)c3n2)C1] | 3-(6-(3-(Dimethylamino)piperidin-1-yl)-1- methyl-1H-pyrazolo[3,4-d]pyrimidin-3-yl)-2,6- difluoro-5-(trifluoromethyl)phenol |
| 18.46 | 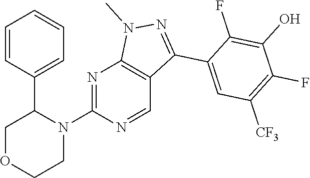 [SMILES: Cn1nc(-c2cc(C(F)(F)F)c(F)c(O)c2F)c2cnc(N3CCOCC3c3ccccc3)nc21] | 2,6-Difluoro-3-(1-methyl-6-(3- phenylmorpholino)-1H-pyrazolo[3,4- d]pyrimidin-3-yl)-5-(trifluoromethyl)phenol |
| 18.47 | 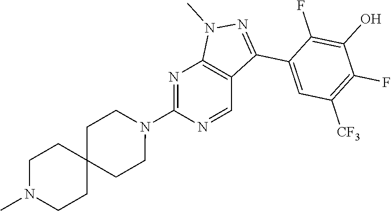 [SMILES: CN1CCC2(CC1)CCN(c1ncc3c(-c4cc(C(F)(F)F)c(F)c(O)c4F)nn(C)c3n1)CC2] | 2,6-Difluoro-3-(1-methyl-6-(9-methyl-3,9- diazaspiro[5.5]undecan-3-yl)-1H-pyrazolo[3,4- d]pyrimidin-3-yl)-5-(trifluoromethyl)phenol |
| 18.48 | 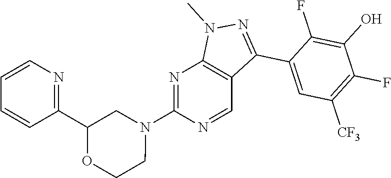 [SMILES: Cn1nc(-c2cc(C(F)(F)F)c(F)c(O)c2F)c2cnc(N3CCOC(c4ccccn4)C3)nc21] | 2,6-Difluoro-3-(1-methyl-6-(2-(pyridin-2- yl)morpholino)-1H-pyrazolo[3,4-d]pyrimidin-3- yl)-5-(trifluoromethyl)phenol |
| 18.49 | 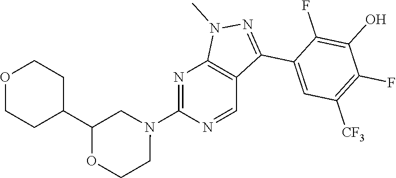 [SMILES: Cn1nc(-c2cc(C(F)(F)F)c(F)c(O)c2F)c2cnc(N3CCOC(C4CCOCC4)C3)nc21] | 2,6-Difluoro-3-(1-methyl-6-(2-(tetrahydro-2H- pyran-4-yl)morpholino)-1H-pyrazolo[3,4- d]pyrimidin-3-yl)-5-(trifluoromethyl)phenol |
| 18.50 | 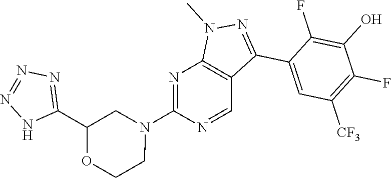 [SMILES: Cn1nc(-c2cc(C(F)(F)F)c(F)c(O)c2F)c2cnc(N3CCOC(c4nnn[nH]4)C3)nc21] | 3-(6-(2-(1H-Tetrazol-5-yl)morpholino)-1- methyl-1H-pyrazolo[3,4-d]pyrimidin-3-yl)-2,6- difluoro-5-(trifluoromethyl)phenol |
| 18.51 | 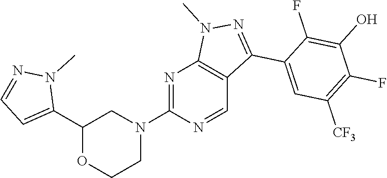 [SMILES: Cn1nccc1C1CN(c2ncc3c(-c4cc(C(F)(F)F)c(F)c(O)c4F)nn(C)c3n2)CCO1] | 2,6-Difluoro-3-(1-methyl-6-(2-(1-methyl-1H- pyrazol-5-yl)morpholino)-1H-pyrazolo[3,4- d]pyrimidin-3-yl)-5-(trifluoromethyl)phenol |
| 18.52 | 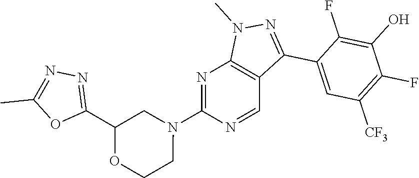 [SMILES: Cc1nnc(C2CN(c3ncc4c(-c5cc(C(F)(F)F)c(F)c(O)c5F)nn(C)c4n3)CCO2)o1] | 2,6-Difluoro-3-(1-methyl-6-(2-(5-methyl-1,3,4- oxadiazol-2-yl)morpholino)-1H-pyrazolo[3,4- d]pyrimidin-3-yl)-5-(trifluoromethyl)phenol |
| 18.53 | 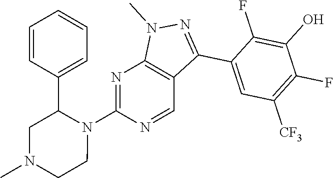 [SMILES: CN1CCN(c2ncc3c(-c4cc(C(F)(F)F)c(F)c(O)c4F)nn(C)c3n2)C(c2ccccc2)C1] | 2,6-Difluoro-3-(1-methyl-6-(4-methyl-2- phenylpiperazin-1-yl)-1H-pyrazolo[3,4- d]pyrimidin-3-yl)-5-(trifluoromethyl)phenol |
| 18.54 | 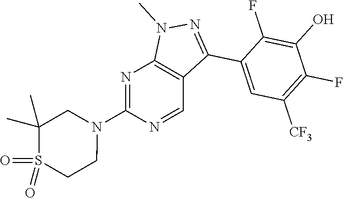 [SMILES: Cn1nc(-c2cc(C(F)(F)F)c(F)c(O)c2F)c2cnc(N3CCS(=O)(=O)C(C)(C)C3)nc21] | 4-(3-(2,4-Difluoro-3-hydroxy-5- (trifluoromethyl)phenyl)-1-methyl-1H- pyrazolo[3,4-d]pyrimidin-6-yl)-2,2- dimethylthiomorpholine 1,1-dioxide |
| 18.55 | 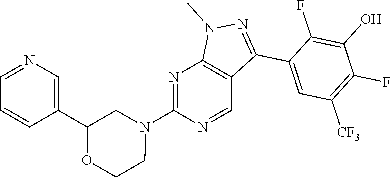 [SMILES: Cn1nc(-c2cc(C(F)(F)F)c(F)c(O)c2F)c2cnc(N3CCOC(c4cccnc4)C3)nc21] | 2,6-Difluoro-3-(1-methyl-6-(2-(pyridin-3- yl)morpholino)-1H-pyrazolo[3,4-d]pyrimidin-3- yl)-5-(trifluoromethyl)phenol |
| 18.56 | 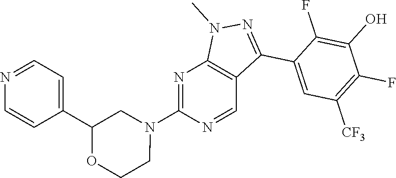 [SMILES: Cn1nc(-c2cc(C(F)(F)F)c(F)c(O)c2F)c2cnc(N3CCOC(c4ccncc4)C3)nc21] | 2,6-Difluoro-3-(1-methyl-6-(2-(pyridin-4- yl)morpholino)-1H-pyrazolo[3,4-d]pyrimidin-3- yl)-5-(trifluoromethyl)phenol |
| 18.57 | 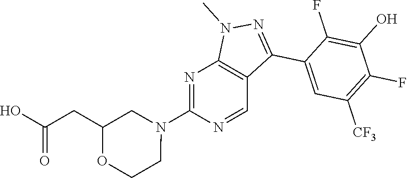 [SMILES: Cn1nc(-c2cc(C(F)(F)F)c(F)c(O)c2F)c2cnc(N3CCOC(CC(=O)O)C3)nc21] | 2-(4-(3-(2,4-Difluoro-3-hydroxy-5- (trifluoromethyl)phenyl)-1-methyl-1H- pyrazolo[3,4-d]pyrimidin-6-yl)morpholin-2- yl)acetic Acid |
| 18.58 | 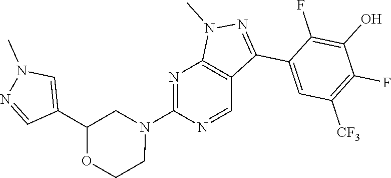 [SMILES: Cn1cc(C2CN(c3ncc4c(-c5cc(C(F)(F)F)c(F)c(O)c5F)nn(C)c4n3)CCO2)cn1] | 2,6-Difluoro-3-(1-methyl-6-(2-(1-methyl-1H- pyrazol-4-yl)morpholino)-1H-pyrazolo[3,4- d]pyrimidin-3-yl)-5-(trifluoromethyl)phenol |
| 18.59 | 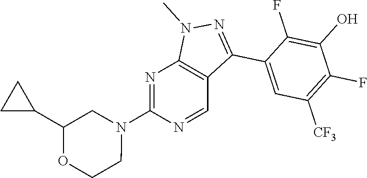 [SMILES: Cn1nc(-c2cc(C(F)(F)F)c(F)c(O)c2F)c2cnc(N3CCOC(C4CC4)C3)nc21] | 3-(6-(2-Cyclopropylmorpholino)-1-methyl-1H- pyrazolo[3,4-d]pyrimidin-3-yl)-2,6-difluoro-5- (trifluoromethyl)phenol |
| 18.60 | 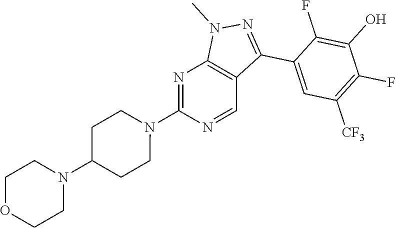 [SMILES: Cn1nc(-c2cc(C(F)(F)F)c(F)c(O)c2F)c2cnc(N3CCC(N4CCOCC4)CC3)nc21] | 2,6-Difluoro-3-(1-methyl-6-(4- morpholinopiperidin-1-yl)-1H-pyrazolo[3,4- d]pyrimidin-3-yl)-5-(trifluoromethyl)phenol |
| 18.61 | 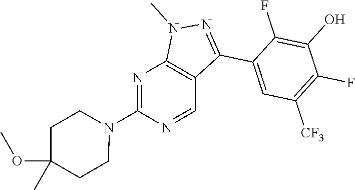 [SMILES: COC1(C)CCN(c2ncc3c(-c4cc(C(F)(F)F)c(F)c(O)c4F)nn(C)c3n2)CC1] | 2,6-Difluoro-3-(6-(4-methoxy-4- methylpiperidin-1-yl)-1-methyl-1H- pyrazolo[3,4-d]pyrimidin-3-yl)-5- (trifluoromethyl)phenol |
| 18.62 | 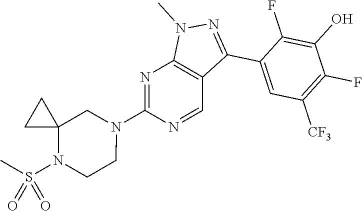 [SMILES: Cn1nc(-c2cc(C(F)(F)F)c(F)c(O)c2F)c2cnc(N3CCN(S(C)(=O)=O)C4(CC4)C3)nc21] | 2,6-Difluoro-3-(1-methyl-6-(4- (methylsulfonyl)-4,7-diazaspiro[2,5]octan-7-yl)- 1H-pyrazolo[3,4-d]pyrimidin-3-yl)-5- (trifluoromethyl)phenol |
| 18.63 | 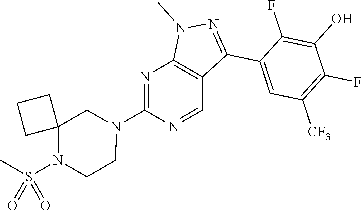 [SMILES: Cn1nc(-c2cc(C(F)(F)F)c(F)c(O)c2F)c2cnc(N3CCN(S(C)(=O)=O)C4(CCC4)C3)nc21] | 2,6-Difluoro-3-(1-methyl-6-(5- (methylsulfonyl)-5,8-diazaspiro[3.5]nonan-8- yl)-1H-pyrazolo[3,4-d]pyrimidin-3-yl)-5- (trifluoromethyl)phenol |
| 18.64 | 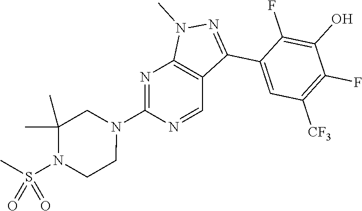 [SMILES: Cn1nc(-c2cc(C(F)(F)F)c(F)c(O)c2F)c2cnc(N3CCN(S(C)(=O)=O)C(C)(C)C3)nc21] | 3-(6-(3,3-Dimethyl-4- (methylsulfonyl)piperazin-1-yl)-1-methyl-1H- pyrazolo[3,4-d]pyrimidin-3-yl)-2,6-difluoro-5- (trifluoromethyl)phenol |
| 18.65 | 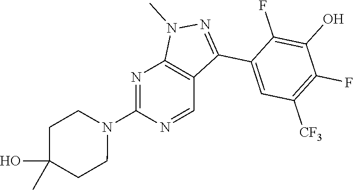 [SMILES: Cn1nc(-c2cc(C(F)(F)F)c(F)c(O)c2F)c2cnc(N3CCC(C)(O)CC3)nc21] | 1-(3-(2,4-Difluoro-3-hydroxy-5- (trifluoromethyl)phenyl)-1-methyl-1H- pyrazolo[3,4-d]pyrimidin-6-yl)-4- methylpiperidin-4-ol |
| 18.66 | 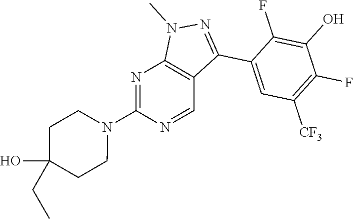 [SMILES: CCC1(O)CCN(c2ncc3c(-c4cc(C(F)(F)F)c(F)c(O)c4F)nn(C)c3n2)CC1] | 1-(3-(2,4-Difluoro-3-hydroxy-5- (trifluoromethyl)phenyl)-1-methyl-1H- pyrazolo[3,4-d]pyrimidin-6-yl)-4- ethylpiperidin-4-ol |
| 18.67 | 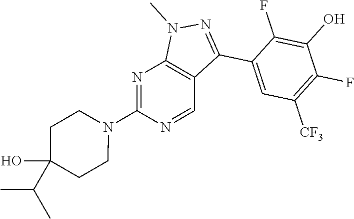 [SMILES: CC(C)C1(O)CCN(c2ncc3c(-c4cc(C(F)(F)F)c(F)c(O)c4F)nn(C)c3n2)CC1] | 1-(3-(2,4-Difluoro-3-hydroxy-5- (trifluoromethyl)phenyl)-1-methyl-1H- pyrazolo[3,4-d]pyrimidin-6-yl)-4- isopropylpiperidin-4-ol |
| 18.68 | 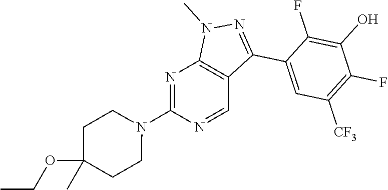 [SMILES: CCOC1(C)CCN(c2ncc3c(-c4cc(C(F)(F)F)c(F)c(O)c4F)nn(C)c3n2)CC1] | 3-(6-(4-Ethoxy-4-methylpiperidin-1-yl)-1- methyl-1H-pyrazolo[3,4-d]pyrimidin-3-yl)-2,6- difluoro-5-(trifluoromethyl)phenol |
| 18.69 | 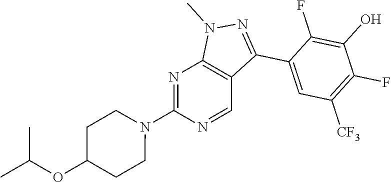 [SMILES: CC(C)OC1CCN(c2ncc3c(-c4cc(C(F)(F)F)c(F)c(O)c4F)nn(C)c3n2)CC1] | 2,6-Difluoro-3-(6-(4-isopropoxypiperidin-1-yl)- 1-methyl-1H-pyrazolo[3,4-d]pyrimidin-3-yl)-5- (trifluoromethyl)phenol |
| 18.70 | 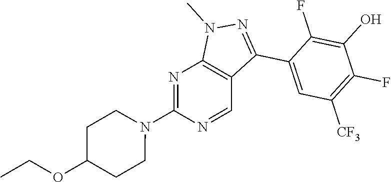 [SMILES: CCOC1CCN(c2ncc3c(-c4cc(C(F)(F)F)c(F)c(O)c4F)nn(C)c3n2)CC1] | 3-(6-(4-Ethoxypiperidin-1-yl)-1-methyl-1H- pyrazolo[3,4-d]pyrimidin-3-yl)-2,6-difluoro-5- (trifluoromethyl)phenol |
| 18.71 | 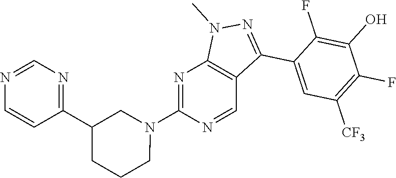 [SMILES: Cn1nc(-c2cc(C(F)(F)F)c(F)c(O)c2F)c2cnc(N3CCCC(c4ccncn4)C3)nc21] | 2,6-Difluoro-3-(1-methyl-6-(3-(pyrimidin-4- yl)piperidin-1-yl)-1H-pyrazolo[3,4-d]pyrimidin- 3-yl)-5-(trifluoromethyl)phenol |
| 18.72 | 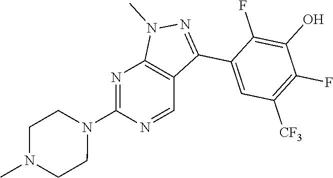 [SMILES: CN1CCN(c2ncc3c(-c4cc(C(F)(F)F)c(F)c(O)c4F)nn(C)c3n2)CC1] | 2,6-Difluroo-3-(1-methyl-6-(4-methylpiperazin- 1-yl)-1H-pyrazolo[3,4-d]pyrimidin-3-yl)-5- (trifluoromethyl)phenol |
| 18.73 | 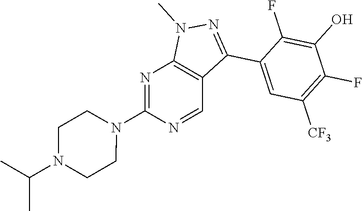 [SMILES: CC(C)N1CCN(c2ncc3c(-c4cc(C(F)(F)F)c(F)c(O)c4F)nn(C)c3n2)CC1] | 2,6-Difluoro-3-(6-(4-isopropylpiperazin-1-yl)- 1-methyl-1H-pyrazolo[3,4-d]pyrimidin-3-yl)-5- (trifluoromethyl)phenol |
| 18.74 | 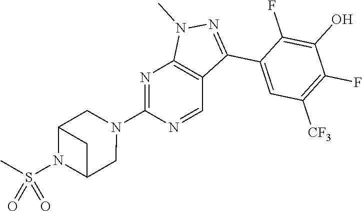 [SMILES: Cn1nc(-c2cc(C(F)(F)F)c(F)c(O)c2F)c2cnc(N3CC4CC(C3)N4S(C)(=O)=O)nc21] | 2,6-Difluoro-3-(1-methyl-6-(6- (methylsulfonyl)-3,6-diazabicyclo[3.1.1]heptan- 3-yl)-1H-pyrazolo[3,4-d]primidin-3-yl)-5- (trifluoromethyl)phenol |
| 18.75 | 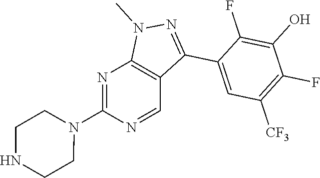 [SMILES: Cn1nc(-c2cc(C(F)(F)F)c(F)c(O)c2F)c2cnc(N3CCNCC3)nc21] | 2,6-Difluoro-3-(1-methyl-6-(piperazin-1-yl)- 1H-pyrazolo[3,4-d]pyrimidin-3-yl)-5- (trifluoromethyl)phenol |
| 18.76 | 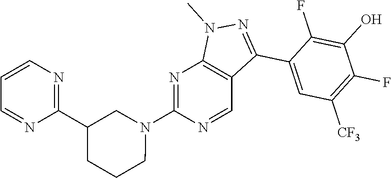 [SMILES: Cn1nc(-c2cc(C(F)(F)F)c(F)c(O)c2F)c2cnc(N3CCCC(c4ncccn4)C3)nc21] | 2,6-Difluoro-3-(1-methyl-6-(3-(pyrimidin-2- yl)piperidin-1-yl)-1H-pyrazolo[3,4-d]pyrimidin- 3-yl)-5-(trifluoromethyl)phenol |
| 18.77 | 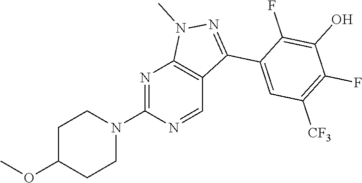 [SMILES: COC1CCN(c2ncc3c(-c4cc(C(F)(F)F)c(F)c(O)c4F)nn(C)c3n2)CC1] | 2,6-Difluoro-3-(6-(4-methoxypiperidin-1-yl)-1- methyl-1H-pyrazolo[3,4-d]pyrimidin-3-yl)-5- (trifluoromethyl)phenol |
| 18.78 | 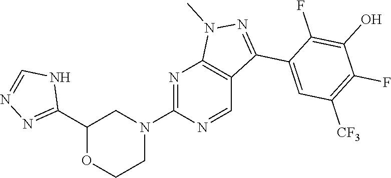 [SMILES: Cn1nc(-c2cc(C(F)(F)F)c(F)c(O)c2F)c2cnc(N3CCOC(c4nnc[nH]4)C3)nc21] | 3-(6-(2-(4H-1,2,4-Triazol-3-yl)morpholino)-1- methyl-1H-pyrazolo[3,4-d]pyrimidin-3-yl)-2,6- difluoro-5-(trifluoromethyl)phenol |
| 18.79 | 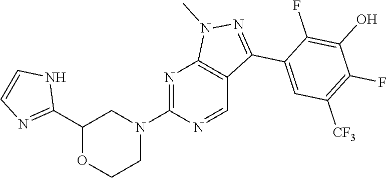 [SMILES: Cn1nc(-c2cc(C(F)(F)F)c(F)c(O)c2F)c2cnc(N3CCOC(c4ncc[nH]4)C3)nc21] | 3-(6-(2-(1H-Imidazol-2-yl)morpholino)-1- methyl-1H-pyrazolo[3,4-d]pyrimdin-3-yl)-2,6- difluoro-5-(trifluoromethyl)phenol |
| 18.80 | 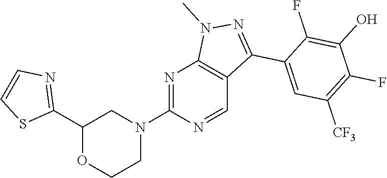 [SMILES: Cn1nc(-c2cc(C(F)(F)F)c(F)c(O)c2F)c2cnc(N3CCOC(c4nccs4)C3)nc21] | 2,6-Difluoro-3-(1-methyl-6-(2-(thiazol-2- yl)morpholino)-1H-pyrazolo[3,4-d]pyrimidin-3- yl)-5-(trifluoromethyl)phenol |
| 18.81 | 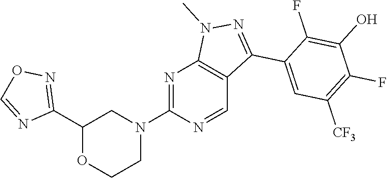 [SMILES: Cn1nc(-c2cc(C(F)(F)F)c(F)c(O)c2F)c2cnc(N3CCOC(c4ncon4)C3)nc21] | 3-(6-(2-(1,2,4-Oxadiazol-3-yl)morpholino)-1- methyl-1H-pyrazolo[3,4-d]pyrimidin-3-yl)-2,6- difluoro-5-(trifluoromethyl)phenol |
| 18.82 | 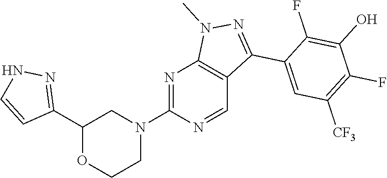 [SMILES: Cn1nc(-c2cc(C(F)(F)F)c(F)c(O)c2F)c2cnc(N3CCOC(c4cc[nH]n4)C3)nc21] | 3-(6-(2-(1H-Pyrazol-3-yl)morpholino)-1- methyl-1H-pyrazolo[3,4-d]pyrimidin-3-yl)-2,6- difluoro-5-(trifluoromethyl)phenol |
| 18.83 | 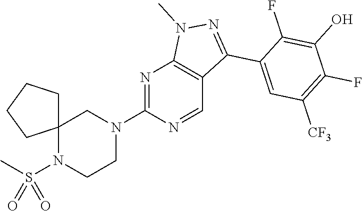 [SMILES: Cn1nc(-c2cc(C(F)(F)F)c(F)c(O)c2F)c2cnc(N3CCN(S(C)(=O)=O)C4(CCCC4)C3)nc21] | 2,6-Difluoro-3-(1-methyl-6-(6- (methylsulfonyl)-6,9-diazaspiro[4.5]decan-9- yl)-1H-pyrazolo[3,4-d]pyrimidin-3-yl)-5- (trifluoromethyl)phenol |
| 18.84 | 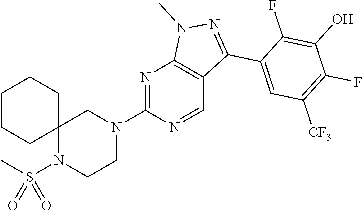 [SMILES: Cn1nc(-c2cc(C(F)(F)F)c(F)c(O)c2F)c2cnc(N3CCN(S(C)(=O)=O)C4(CCCCC4)C3)nc21] | 2,6-Difluoro-3-(1-methyl-6-(1- (methylsulfonyl)-1,4-diazaspiro[5.5]undecan-4- yl)-1H-pyrazolo[3,4-d]pyrimidin-3-yl)-5- (trifluoromethyl)phenol |
| 18.85 | 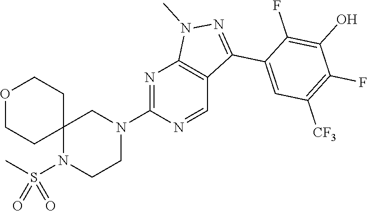 [SMILES: Cn1nc(-c2cc(C(F)(F)F)c(F)c(O)c2F)c2cnc(N3CCN(S(C)(=O)=O)C4(CCOCC4)C3)nc21] | 2,6-Difluoro-3-(1-methyl-6-(1- (methylsulfonyl)-9-oxa-1,4- diazaspiro[5.5]undecan-4-yl)-1H-pyrazolo[3,4- d]pyrimidin-3-yl)-5-(trifluoromethyl)phenol |
| 18.86 | 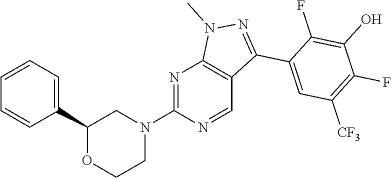 [SMILES: Cn1nc(-c2cc(C(F)(F)F)c(F)c(O)c2F)c2cnc(N3CCO[C@@H](c4ccccc4)C3)nc21] | (S)-2,6-Difluoro-3-(1-methyl-6-(2- phenylmorpholino)-1H-pyrazolo[3,4- d]pyrimidin-3-yl)-5-(trifluoromethyl)phenol |
| 18.87 | 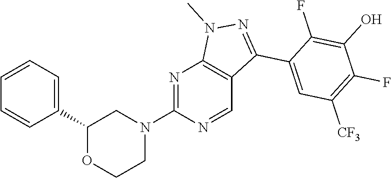 [SMILES: Cn1nc(-c2cc(C(F)(F)F)c(F)c(O)c2F)c2cnc(N3CCO[C@H](c4ccccc4)C3)nc21] | (R)-2,6-Difluoro-3-(1-methyl-6-(2- phenylmorpholino)-1H-pyrazolo[3,4- d]pyrimidin-3-yl)-5-(trifluoromethyl)phenol |
| 18.88 | 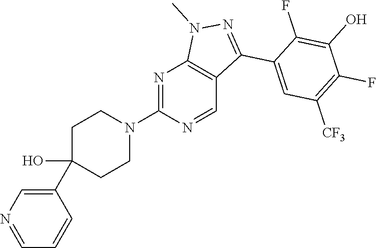 [SMILES: Cn1nc(-c2cc(C(F)(F)F)c(F)c(O)c2F)c2cnc(N3CCC(O)(c4cccnc4)CC3)nc21] | 1-(3-(2,4-Difluoro-3-hydroxy-5- (trifluoromethyl)phenyl)-1-methyl-1H- pyrazolo[3,4-d]pyrimidin-6-yl)-4-(pyridin-3- yl)piperidin-4-ol |
| 18.89 | 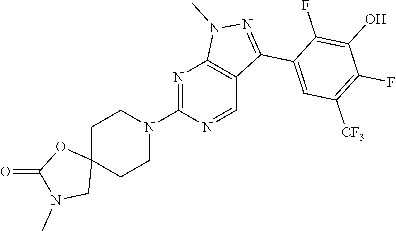 [SMILES: CN1CC2(CCN(c3ncc4c(-c5cc(C(F)(F)F)c(F)c(O)c5F)nn(C)c4n3)CC2)OC1=O] | 8-(3-(2,4-Difluoro-3-hydroxy-5- (trifluoromethyl)phenyl)-1-methyl-1H- pyrazolo[3,4-d]pyrimidin-6-yl)-3-methyl-1-oxa- 3,8-diazaspiro[4.5]decan-2-one |
| 18.90 | 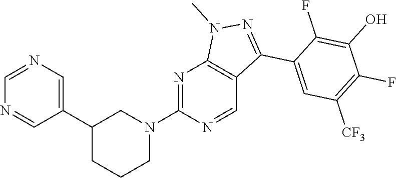 [SMILES: Cn1nc(-c2cc(C(F)(F)F)c(F)c(O)c2F)c2cnc(N3CCCC(c4cncnc4)C3)nc21] | 2,6-Difluoro-3-(1-methyl-6-(3-(pyrimidin-5- yl)piperidin-1-yl)-1H-pyrazolo[3,4-d]pyrimidin- 3-yl)-5-(trifluoromethyl)phenol |
| 18.91 | 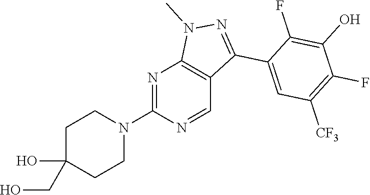 [SMILES: Cn1nc(-c2cc(C(F)(F)F)c(F)c(O)c2F)c2cnc(N3CCC(O)(CO)CC3)nc21] | 1-(3-(2,4-Difluoro-3-hydroxy-5- (trifluoromethyl)phenyl)-1-methyl-1H- pyrazolo[3,4-d]pyrimidin-6-yl)-4- (hydroxymethyl)piperidin-4-ol |
| 18.92 | 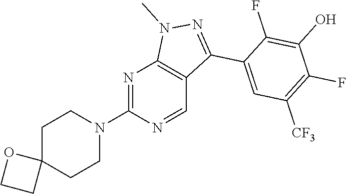 [SMILES: Cn1nc(-c2cc(C(F)(F)F)c(F)c(O)c2F)c2cnc(N3CCC4(CCO4)CC3)nc21] | 2,6-Difluoro-3-(1-methyl-6-(1-oxa-7- azaspiro[3.5]nonan-7-yl)-1H-pyrazolo[3,4- d]pyrimidin-3-yl)-5-(trifluoromethyl)phenol |
| 18.93 | 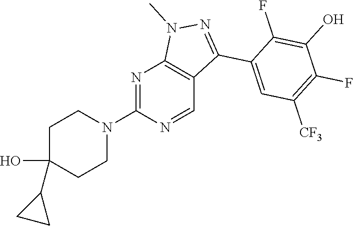 [SMILES: Cn1nc(-c2cc(C(F)(F)F)c(F)c(O)c2F)c2cnc(N3CCC(O)(C4CC4)CC3)nc21] | 4-Cyclopropyl-1-(3-(2,4-difluoro-3-hydroxy-5- (trifluoromethyl)phenyl)-1-methyl-1H- pyrazolo[3,4-d]pyrimidin-6-yl)piperidin-4-ol |
| 18.94 | 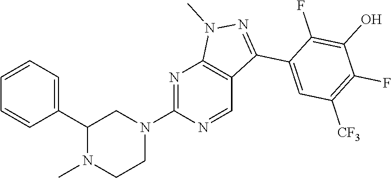 [SMILES: CN1CCN(c2ncc3c(-c4cc(C(F)(F)F)c(F)c(O)c4F)nn(C)c3n2)CC1c1ccccc1] | 2,6-Difluoro-3-(1-methyl-6-(4-methyl-3- phenylpiperazin-1-yl)-1H-pyrazolo[3,4- d]pyrimidin-2-yl)-5-(trifluoromethyl)phenol |
| 18.95 | 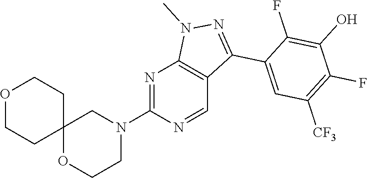 [SMILES: Cn1nc(-c2cc(C(F)(F)F)c(F)c(O)c2F)c2cnc(N3CCOC4(CCOCC4)C3)nc21] | 2,6-Difluoro-3-(1-methyl-6-(1,9-dioxa-4- azaspiro[5.5]undecan-4-yl)-1H-pyrazolo[3,4- d]pyrimidin-3-yl)-5-(trifluoromethyl)phenol |
| 18.96 | 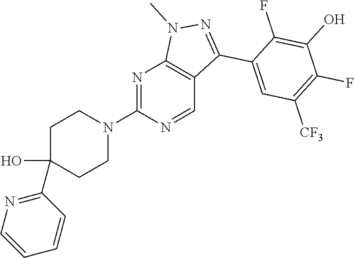 [SMILES: Cn1nc(-c2cc(C(F)(F)F)c(F)c(O)c2F)c2cnc(N3CCC(O)(c4ccccn4)CC3)nc21] | 1-(3-(2,4-Difluoro-3-hydroxy-5- (trifluoromethyl)phenyl)-1-methyl-1H- pyrazolo[3,4-d]pyrimidin-6-yl)-4-(pyridin-2- yl)piperidin-4-ol |
| 18.97 | 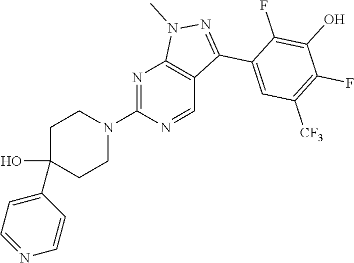 [SMILES: Cn1nc(-c2cc(C(F)(F)F)c(F)c(O)c2F)c2cnc(N3CCC(O)(c4ccncc4)CC3)nc21] | 1-(3-(2,4-Difluoro-3-hydroxy-5- (trifluoromethyl)phenyl)-1-methyl-1H- pyrazolo[3,4-d]pyrimidin-6-yl)-4-(pyridin-4- yl)piperidin-4-ol |
| 18.98 | 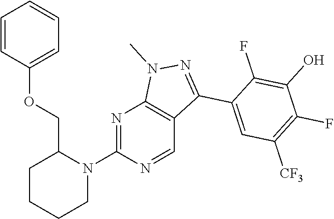 [SMILES: Cn1nc(-c2cc(C(F)(F)F)c(F)c(O)c2F)c2cnc(N3CCCCC3COc3ccccc3)nc21] | 2,6-Difluoro-3-(1-methyl-6-(2- (phenoxymethyl)piperidin-1-yl)-1H- pyrazolo[3,4-d]pyrimidin-3-yl)-5- (trifluoromethyl)phenol |
| 18.99 | 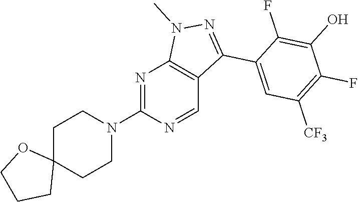 [SMILES: Cn1nc(-c2cc(C(F)(F)F)c(F)c(O)c2F)c2cnc(N3CCC4(CCCO4)CC3)nc21] | 2,6-Difluoro-3-(1-methyl-6-(1-oxa-8- azaspiro[4.5]decan-8-yl)-1H-pyrazolo[3,4- d]pyrimidin-3-yl)-5-(trifluoromethyl)phenol |
| 18.100 | 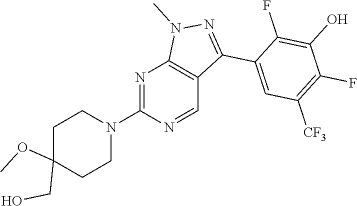 [SMILES: COC1(CO)CCN(c2ncc3c(-c4cc(C(F)(F)F)c(F)c(O)c4F)nn(C)c3n2)CC1] | 2,6-Difluoro-3-(6-(4-(hydroxymethyl)-4- methoxypiperidin-1-yl)-1-methyl-1H- pyrazolo[3,4-d]pyrimidin-3-yl)-5- (trifluoromethyl)phenol |
| 18.101 | 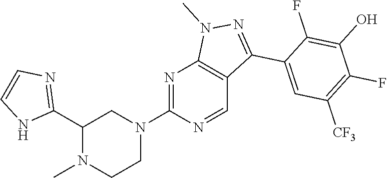 [SMILES: CN1CCN(c2ncc3c(-c4cc(C(F)(F)F)c(F)c(O)c4F)nn(C)c3n2)CC1c1ncc[nH]1] | 3-(6-(3-(1H-(Imidazol-2-yl)-4-methylpiperazin- 1-yl)-1-methyl-1H-pyrazolo[3,4-d]pyrimidin-3- yl)-2,6-difluoro-5-(trifluoromethyl)phenol |
| 18.102 | 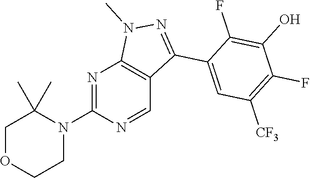 [SMILES: Cn1nc(-c2cc(C(F)(F)F)c(F)c(O)c2F)c2cnc(N3CCOCC3(C)C)nc21] | 3-(6-(3,3-Dimethylmorpholino)-1-methyl-1H- pyrazolo[3,4-d]pyrimidin-3-yl)-2,6-difluoro-5- (trifluoromethyl)phenol |
| 18.103 | 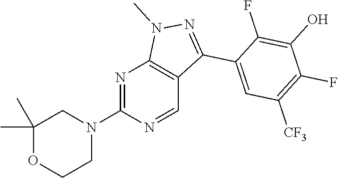 [SMILES: Cn1nc(-c2cc(C(F)(F)F)c(F)c(O)c2F)c2cnc(N3CCOC(C)(C)C3)nc21] | 3-(6-(2,2-Dimethylmorpholino)-1-methyl-1H- pyrazolo[3,4-d]pyrimidin-3-yl)-2,6-difluoro-5- (trifluoromethyl)phenol |
| 18.104 | 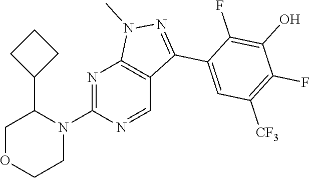 [SMILES: Cn1nc(-c2cc(C(F)(F)F)c(F)c(O)c2F)c2cnc(N3CCOCC3C3CCC3)nc21] | 3-(6-(3-Cyclobutylmorpholino)-1-methyl-1H- pyrazolo[3,4-d]pyrimidin-3-yl)-2,6-difluoro-5- (trifluoromethyl)phenol |
| 18.105 | 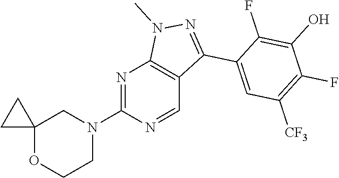 [SMILES: Cn1nc(-c2cc(C(F)(F)F)c(F)c(O)c2F)c2cnc(N3CCOC4(CC4)C3)nc21] | 2,6-Difluoro-3-(1-methyl-6-(4-oxa-7- azaspiro[2.5]octan-7-yl)-1H-pyrazolo[3,4- d]pyrimidin-3-yl)-5-(trifluoromethyl)phenol |
| 18.106 | 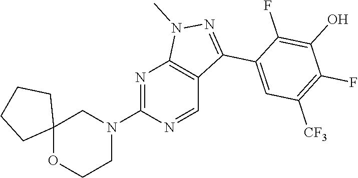 [SMILES: Cn1nc(-c2cc(C(F)(F)F)c(F)c(O)c2F)c2cnc(N3CCOC4(CCCC4)C3)nc21] | 2,6-Difluoro-3-(1-methyl-6-(6-oxa-9- azaspiro[4.5]decan-9-yl)-1H-pyrazolo[3,4- d]pyrimidin-3-yl)-5-(trifluoromethyl)phenol |
| 18.107 | 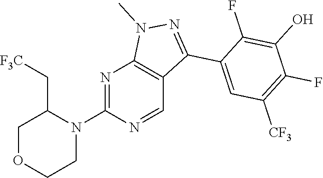 [SMILES: Cn1nc(-c2cc(C(F)(F)F)c(F)c(O)c2F)c2cnc(N3CCOCC3CC(F)(F)F)nc21] | 2,6-Difluoro-3-(1-methyl-6-(3-(2,2,2- trifluoroethyl)morpholino)-1H-pyrazolo[3,4- d]pyrimidin-3-yl)-5-(trifluoromethyl)phenol |
| 18.108 | 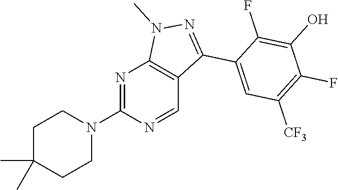 [SMILES: Cn1nc(-c2cc(C(F)(F)F)c(F)c(O)c2F)c2cnc(N3CCC(C)(C)CC3)nc21] | 3-(6-(4,4-Dimethylpiperidin-1-yl)-1-methyl-1H- pyrazolo[3,4-d]pyrimidin-3-yl)-2,6-difluoro-5- (trifluoromethyl)phenol |
| 18.109 | 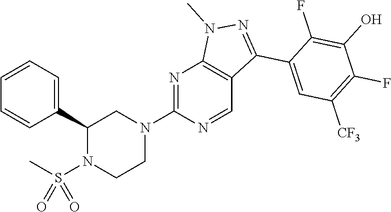 [SMILES: Cn1nc(-c2cc(C(F)(F)F)c(F)c(O)c2F)c2cnc(N3CCN(S(C)(=O)=O)[C@@H](c4ccccc4)C3)nc21] | (S)-2,6-Difluoro-3-(1-methyl-6-(4- (methylsulfonyl)-3-phenylpiperazin-1-yl)-1H- pyrazolo[3,4-d]pyrimidin-3-yl)-5- (trifluoromethyl)phenol |
| 18.110 | 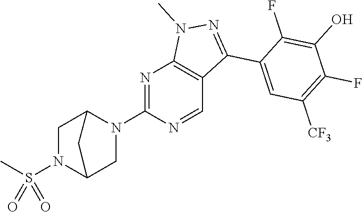 [SMILES: Cn1nc(-c2cc(C(F)(F)F)c(F)c(O)c2F)c2cnc(N3CC4CC3CN4S(C)(=O)=O)nc21] | 2,6-Difluoro-3-(1-methyl-6-(5- (methylsulfonyl)-2,5-diazabicyclo[2.2.1]heptan- 2-yl)-1H-pyrazolo[3,4-d]pyrimidin-3-yl)-5- (trifluoromethyl)phenol |
| 18.111 | 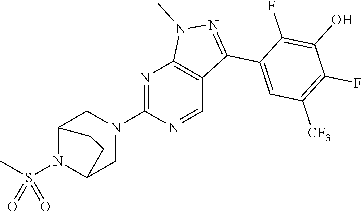 [SMILES: Cn1nc(-c2cc(C(F)(F)F)c(F)c(O)c2F)c2cnc(N3CC4CCC(C3)N4S(C)(=O)=O)nc21] | 2,6-Difluoro-3-(1-methyl-6-(8- (methylsulfonyl)-3,8-diazabicyclo[3.2.1]octan- 3-yl)-1H-pyrazolo[3,4-d]pyrimidin-3-yl)-5- (trifluoromethyl)phenol |
| 18.112 | 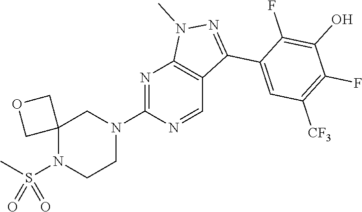 [SMILES: Cn1nc(-c2cc(C(F)(F)F)c(F)c(O)c2F)c2cnc(N3CCN(S(C)(=O)=O)C4(COC4)C3)nc21] | 2,6-Difluoro-3-(1-methyl-6-(5- (methylsulfonyl)-2-oxa-5,8- diazaspiro[3.5]nonan-8-yl)-1H-pyrazolo[3,4- d]pyrimidin-3-yl)-5-(trifluoromethyl)phenol |
| 18.113 | 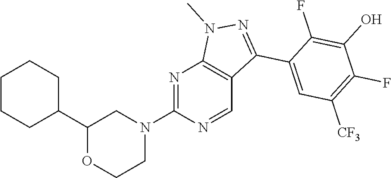 [SMILES: Cn1nc(-c2cc(C(F)(F)F)c(F)c(O)c2F)c2cnc(N3CCOC(C4CCCCC4)C3)nc21] | 3-(6-(2-Cyclohexylmorpholino)-1-methyl-1H- pyrazolo[3,4-d]pyrimidin-3-yl)-2,6-difluoro-5- (trifluoromethyl)phenol |
| 18.114 | 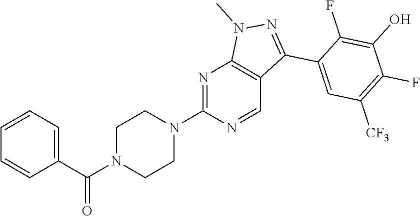 [SMILES: Cn1nc(-c2cc(C(F)(F)F)c(F)c(O)c2F)c2cnc(N3CCN(C(=O)c4ccccc4)CC3)nc21] | (4-(3-(2,4-Difluoro-3-hydroxy-5- (trifluoromethyl)phenyl)-1-methyl-1H- pyrazolo[3,4-d]pyrimidin-6-yl)piperazin-1- yl)(phenyl)methanone |
| 18.115 | 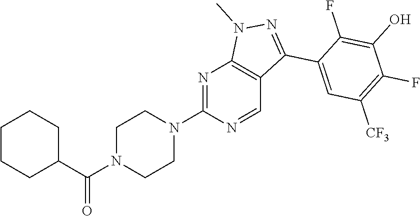 [SMILES: Cn1nc(-c2cc(C(F)(F)F)c(F)c(O)c2F)c2cnc(N3CCN(C(=O)C4CCCCC4)CC3)nc21] | Cyclohexyl(4-(3-(2,4-difluoro-3-hydroxy-5- (trifluoromethyl)phenyl)-1-methyl-1H- pyrazolo[3,4-d]pyrimidin-6-yl)piperazin-1- yl)methanone |
| 18.116 | 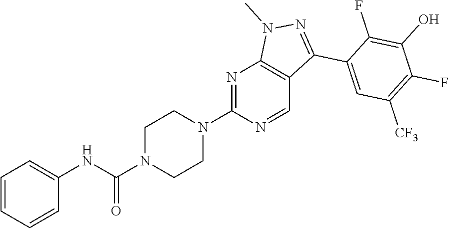 [SMILES: Cn1nc(-c2cc(C(F)(F)F)c(F)c(O)c2F)c2cnc(N3CCN(C(=O)Nc4ccccc4)CC3)nc21] | 4-(3-(2,4-Difluoro-3-hydroxy-5- (trifluoromethyl)phenyl)-1-methyl-1H- pyrazolo[3,4-d]pyrimidin-6-yl)-N- phenylpiperazine-1-carboxamide |
| 18.117 | 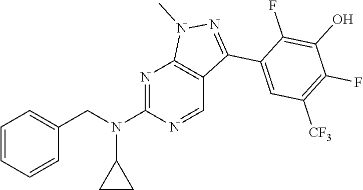 [SMILES: Cn1nc(-c2cc(C(F)(F)F)c(F)c(O)c2F)c2cnc(N(Cc3ccccc3)C3CC3)nc21] | 3-(6-(Benzyl(cyclopropyl)amino)-1-methyl-1H- pyrazolo[3,4-d]pyrimidin-3-yl)-2,6-difluoro-5- (trifluoromethyl)phenol |
| 18.118 | 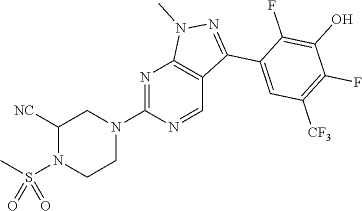 [SMILES: Cn1nc(-c2cc(C(F)(F)F)c(F)c(O)c2F)c2cnc(N3CCN(S(C)(=O)=O)C(C#N)C3)nc21] | 4-(3-(2,4-Difluoro-3-hydroxy-5- (trifluoromethyl)phenyl)-1-methyl-1H- pyrazolo[3,4-d]pyrimidin-6-yl)-1- (methylsulfonyl)piperazine-2-carbonitrile |
| 18.119 | 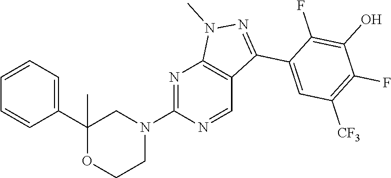 [SMILES: Cn1nc(-c2cc(C(F)(F)F)c(F)c(O)c2F)c2cnc(N3CCOC(C)(c4ccccc4)C3)nc21] | 2,6-Difluoro-3-(1-methyl-6-(2-methyl-2- phenylmorpholino)-1H-pyrazolo[3,4- d]pyrimidin-3-yl)-5-(trifluoromethyl)phenol |
| 18.120 | 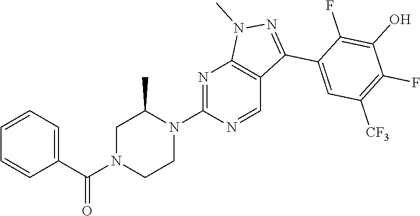 [SMILES: C[C@@H]1CN(C(=O)c2ccccc2)CCN1c1ncc2c(-c3cc(C(F)(F)F)c(F)c(O)c3F)nn(C)c2n1] | (R)-(4-(3-(2,4-Difluoro-3-hydroxy-5- (trifluoromethyl)phenyl)-1-methyl-1H- pyrazolo[3,4-d]pyrimidin-6-yl)-3- methylpiperazin-1-yl)(phenyl)methanone |
| 18.121 | 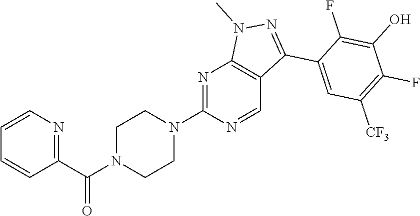 [SMILES: Cn1nc(-c2cc(C(F)(F)F)c(F)c(O)c2F)c2cnc(N3CCN(C(=O)c4ccccn4)CC3)nc21] | (4-(3-(2,4-Difluoro-3-hydroxy-5- (trifluoromethyl)phenyl)-1-methyl-1H- pyrazolo[3,4-d]pyrimidin-6-yl)piperazin-1- yl)(pyridin-2-yl)methanone |
| 18.122 | 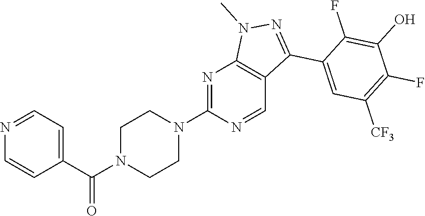 [SMILES: Cn1nc(-c2cc(C(F)(F)F)c(F)c(O)c2F)c2cnc(N3CCN(C(=O)c4ccncc4)CC3)nc21] | (4-(3-(2,4-Difluoro-3-hydroxy-5- (trifluoromethyl)phenyl)-1-methyl-1H- pyrazolo[3,4-d]pyrimidin-6-yl)piperazin-1- yl)(pyridin-4-yl)methanone |
| 18.123 | 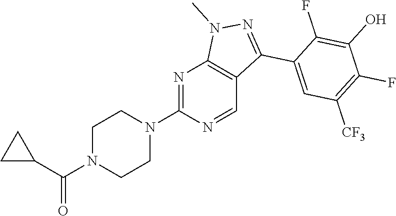 [SMILES: Cn1nc(-c2cc(C(F)(F)F)c(F)c(O)c2F)c2cnc(N3CCN(C(=O)C4CC4)CC3)nc21] | Cyclopropyl(4-(3-(2,4-difluoro-3-hydroxy-5- (trifluoromethyl)phenyl)-1-methyl-1H- pyrazolo[3,4-d]pyrimidin-6-yl)piperazin-1- yl)methanone |
| 18.124 | 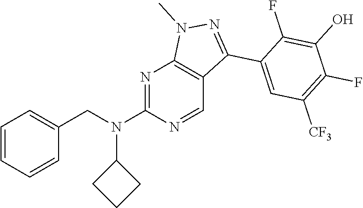 [SMILES: Cn1nc(-c2cc(C(F)(F)F)c(F)c(O)c2F)c2cnc(N(Cc3ccccc3)C3CCC3)nc21] | 3-(6-(Benzyl(cyclobutyl)amino)-1-methyl-1H- pyrazolo[3,4-d]pyrimidin-3-yl)-2,6-difluoro-5- (trifluoromethyl)phenol |
| 18.125 | 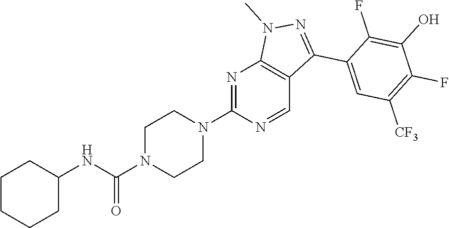 [SMILES: Cn1nc(-c2cc(C(F)(F)F)c(F)c(O)c2F)c2cnc(N3CCN(C(=O)NC4CCCCC4)CC3)nc21] | N-Cyclohexyl-4-(3-(2,4-difluoro-3-hydroxy-5- (trifluoromethyl)phenyl)-1-methyl-1H- pyrazolo[3,4-d]pyrimidin-6-yl)piperazine-1- carboxamide |
| 18.126 | 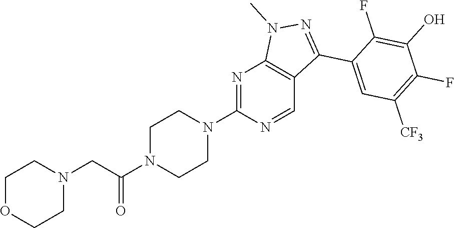 [SMILES: Cn1nc(-c2cc(C(F)(F)F)c(F)c(O)c2F)c2cnc(N3CCN(C(=O)CN4CCOCC4)CC3)nc21] | 1-(4-(3-(2,4-Difluoro-3-hydroxy-5- (trifluoromethyl)phenyl)-1-methyl-1H- pyrazolo[3,4-d]pyrimidin-6-yl)piperazin-1-yl)- 2-morpholinoethan-1-one |
| 18.127 | 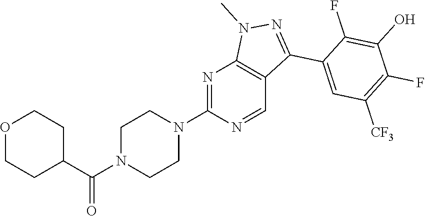 [SMILES: Cn1nc(-c2cc(C(F)(F)F)c(F)c(O)c2F)c2cnc(N3CCN(C(=O)C4CCOCC4)CC3)nc21] | (4-(3-(2,4-Difluoro-3-hydroxy-5- (trifluoromethyl)phenyl)-1-methyl-1H- pyrazolo[3,4-d]pyrimidin-6-yl)piperazin-1- yl)(tetrahydro-2H-pyran-4-yl)methanone |
| 18.128 | 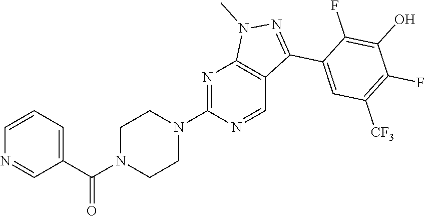 [SMILES: Cn1nc(-c2cc(C(F)(F)F)c(F)c(O)c2F)c2cnc(N3CCN(C(=O)c4cccnc4)CC3)nc21] | (4-(3-(2,4-Difluoro-3-hydroxy-5- (trifluoromethyl)phenyl)-1-methyl-1H- pyrazolo[3,4-d]pyrimidin-6-yl)piperazin-1- yl)(pyridin-3-yl)methanone |
| 18.129 | 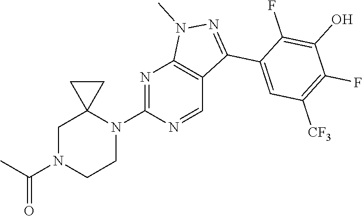 [SMILES: CC(=O)N1CCN(c2ncc3c(-c4cc(C(F)(F)F)c(F)c(O)c4F)nn(C)c3n2)C2(CC2)C1] | 1-(4-(3-(2,4-Difluoro-3-hydroxy-5- (trifluoromethyl)phenyl)-1-methyl-1H- pyrazolo[3,4-d]pyrimidin-6-yl)-4,7- diazaspiro[2.5]octan-7-yl)ethan-1-one |
| 18.130 | 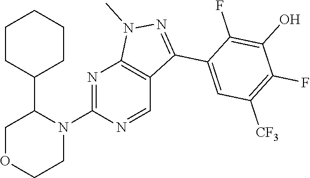 [SMILES: Cn1nc(-c2cc(C(F)(F)F)c(F)c(O)c2F)c2cnc(N3CCOCC3C3CCCCC3)nc21] | 3-(6-(3-Cyclohexylmorpholino)-1-methyl-1H- pyrazolo[3,4-d]pyrimidin-3-yl)-2,6-difluoro-5- (trifluoromethyl)phenol |
| 18.131 | 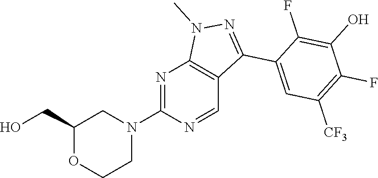 [SMILES: Cn1nc(-c2cc(C(F)(F)F)c(F)c(O)c2F)c2cnc(N3CCO[C@@H](CO)C3)nc21] | (R)-2,6-Difluoro-3-(6-(2- (hydroxymethyl)morpholino)-1-methyl-1H- pyrazolo[3,4-d]pyrimidin-3-yl)-5- (trifluoromethyl)phenol |
| 18.132 | 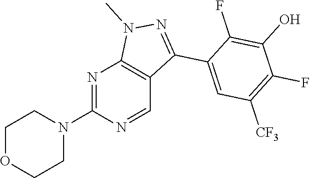 [SMILES: Cn1nc(-c2cc(C(F)(F)F)c(F)c(O)c2F)c2cnc(N3CCOCC3)nc21] | 2,6-Difluoro-3-(1-methyl-6-morpholino-1H- pyrazolo[3,4-d]pyrimidin-3-yl)-5- (trifluoromethyl)phenol |
| 19 | 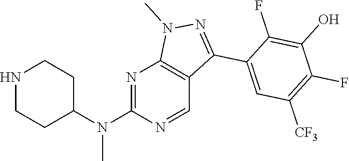 [SMILES: CN(c1ncc2c(-c3cc(C(F)(F)F)c(F)c(O)c3F)nn(C)c2n1)C1CCNCC1] | 2,6-Difluoro-3-(1-methyl-6-(methyl(piperidin- 4-yl)amino)-1H-pyrazolo[3,4-d]pyrimidin-3- yl)-5-(trifluoromethyl)phenol |
| 19.01 | 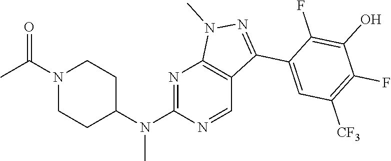 [SMILES: CC(=O)N1CCC(N(C)c2ncc3c(-c4cc(C(F)(F)F)c(F)c(O)c4F)nn(C)c3n2)CC1] | 1-(4-((3-(2,4-Difluoro-3-hydroxy-5- (trifluoromethyl)phenyl)-1-methyl-1H- pyrazolo[3,4-d]pyrimidin-6- yl)(methyl)amino)piperidin-1-yl)ethan-1-one |
| 19.02 | 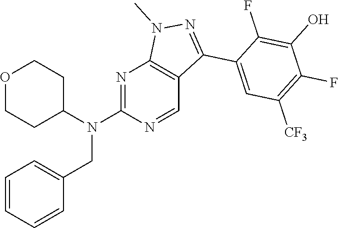 [SMILES: Cn1nc(-c2cc(C(F)(F)F)c(F)c(O)c2F)c2cnc(N(Cc3ccccc3)C3CCOCC3)nc21] | 3-(6-(Benzyl(tetrahydro-2H-pyran-4-yl)amino)- 1-methyl-1H-pyrazolo[3,4-d]pyrimidin-3-yl)- 2,6-difluoro-5-(trifluoromethyl)phenol |
| 20 | 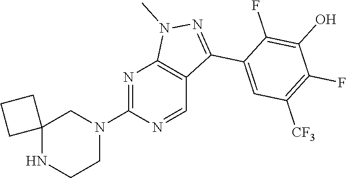 [SMILES: Cn1nc(-c2cc(C(F)(F)F)c(F)c(O)c2F)c2cnc(N3CCNC4(CCC4)C3)nc21] | 2,6-Difluoro-3-(1-methyl-6-(5,8- diazaspiro[3.5]nonan-8-yl)-1H-pyrazolo[3,4- d]pyrimidin-3-yl)-5-(trifluoromethyl)phenol HCl salt |
| 20.01 | 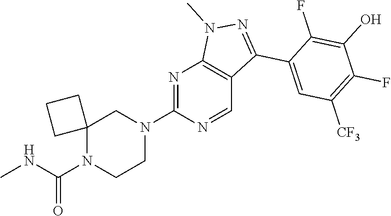 [SMILES: CNC(=O)N1CCN(c2ncc3c(-c4cc(C(F)(F)F)c(F)c(O)c4F)nn(C)c3n2)CC12CCC2] | 8-(3-(2,4-Difluoro-3-hydroxy-5- (trifluoromethyl)phenyl)-1-methyl-1H- pyrazolo[3,4-d]pyrimidin-6-yl)-N-methyl-5,8- diazaspiro[3.5]nonane-5-carboxamide |
| 20.02 | 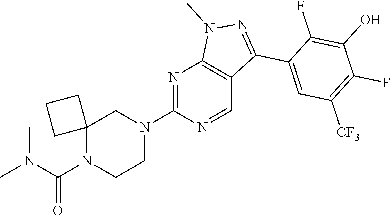 [SMILES: CN(C)C(=O)N1CCN(c2ncc3c(-c4cc(C(F)(F)F)c(F)c(O)c4F)nn(C)c3n2)CC12CCC2] | 8-(3-(2,4-Difluoro-3-hydroxy-5- (trifluoromethyl)phenyl)-1-methyl-1H- pyrazolo[3,4-d]pyrimidin-6-yl)-N,N-dimethyl- 5,8-diazaspiro[3.5]nonane-5-carboxamide |
| 20.03 | 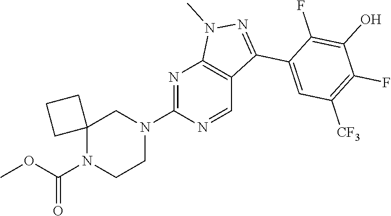 [SMILES: COC(=O)N1CCN(c2ncc3c(-c4cc(C(F)(F)F)c(F)c(O)c4F)nn(C)c3n2)CC12CCC2] | Methyl 8-(3-(2,4-difluoro-3-hydroxy-5- (trifluoromethyl)phenyl)-1-methyl-1H- pyrazolo[3,4-d]pyrimidin-6-yl)-5,8- diazaspiro[3.5]nonane-5-carboxylate |
| 21 | 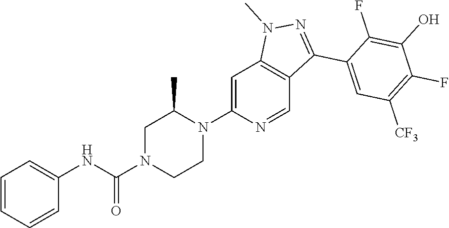 [SMILES: C[C@@H]1CN(C(=O)Nc2ccccc2)CCN1c1cc2c(cn1)c(-c1cc(C(F)(F)F)c(F)c(O)c1F)nn2C] | (R)-4-(3-(2,4-Difluoro-3-hydroxy-5- (trifluoromethyl)phenyl)-1-methyl-1H- pyrazolo[4,3-c]pyridin-6-yl)-3-methyl-N- phenylpiperazine-1-carboxamide |
| 21.01 | 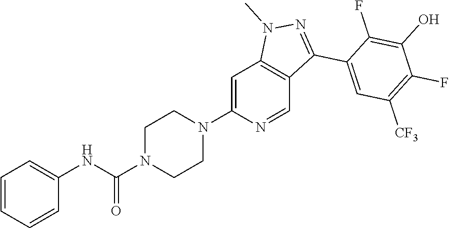 [SMILES: Cn1nc(-c2cc(C(F)(F)F)c(F)c(O)c2F)c2cnc(N3CCN(C(=O)Nc4ccccc4)CC3)cc21] | 4-(3-(2,4-Difluoro-3-hydroxy-5- (trifluoromethyl)phenyl)-1-methyl-1H- pyrazolo[4,3-c]pyridin-6-yl)-N- phenylpiperazine-1-carboxamide |
| 21.02 | 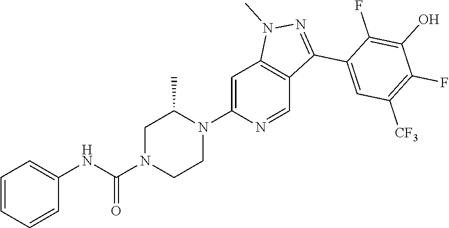 [SMILES: C[C@H]1CN(C(=O)Nc2ccccc2)CCN1c1cc2c(cn1)c(-c1cc(C(F)(F)F)c(F)c(O)c1F)nn2C] | (S)-4-(3-(2,4-Difluoro-3-hydroxy-5- (trifluoromethyl)phenyl)-1-methyl-1H- pyrazolo[4,3-c]pyridin-6-yl)-3-methyl-N- phenylpiperazine-1-carboxamide |
| 21.03 | 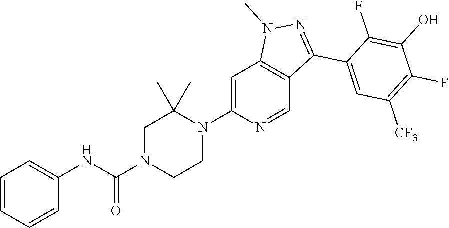 [SMILES: Cn1nc(-c2cc(C(F)(F)F)c(F)c(O)c2F)c2cnc(N3CCN(C(=O)Nc4ccccc4)CC3(C)C)cc21] | 4-(3-(2,4-Difluoro-3-hydroxy-5- (trifluoromethyl)phenyl)-1-methyl-1H- pyrazolo[4,3-c]pyridin-6-yl)-3,3-dimethyl-N- phenylpiperazine-1-carboxamide |
| 21.04 | 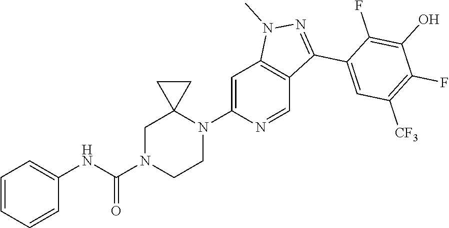 [SMILES: Cn1nc(-c2cc(C(F)(F)F)c(F)c(O)c2F)c2cnc(N3CCN(C(=O)Nc4ccccc4)CC34CC4)cc21] | 4-(3-(2,4-Difluoro-3-hydroxy-5- (trifluoromethyl)phenyl)-1-methyl-1H- pyrazolo[4,3-c]pyridin-6-yl)-N-phenyl-4,7- diazaspiro[2.5]octane-7-carboxamide |
| 21.05 | 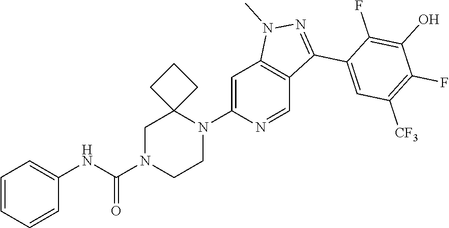 [SMILES: Cn1nc(-c2cc(C(F)(F)F)c(F)c(O)c2F)c2cnc(N3CCN(C(=O)Nc4ccccc4)CC34CCC4)cc21] | 5-(3-(2,4-Difluoro-3-hydroxy-5- (trifluoromethyl)phenyl)-1-methyl-1H- pyrazolo[4,3-c]pyridin-6-yl)-N-phenyl-5,8- diazaspiro[3.5]nonane-8-carboxamide |
| 21.06 | 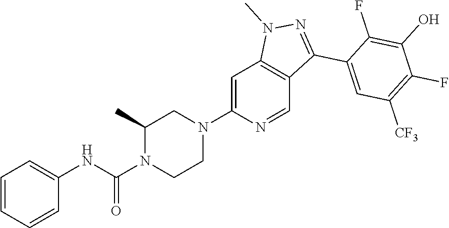 [SMILES: C[C@H]1CN(c2cc3c(cn2)c(-c2cc(C(F)(F)F)c(F)c(O)c2F)nn3C)CCN1C(=O)Nc1ccccc1] | (S)-4-(3-(2,4-Difluoro-3-hydroxy-5- (trifluoromethyl)phenyl)-1-methyl-1H- pyrazolo[4,3-c]pyridin-6-yl)-2-methyl-N- phenylpiperazine-1-carboxamide |
| 21.07 | 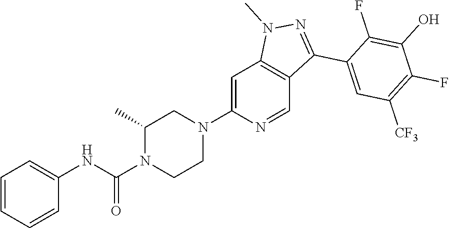 [SMILES: C[C@@H]1CN(c2cc3c(cn2)c(-c2cc(C(F)(F)F)c(F)c(O)c2F)nn3C)CCN1C(=O)Nc1ccccc1] | (R)-4-(3-(2,4-Difluoro-3-hydroxy-5- (trifluoromethyl)phenyl)-1-methyl-1H- pyrazolo[4,3-c]pyridin-6-yl)-2-methyl-N- phenylpiperazine-1-carboxamide |
| 21.08 | 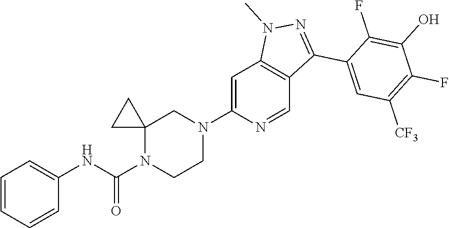 [SMILES: Cn1nc(-c2cc(C(F)(F)F)c(F)c(O)c2F)c2cnc(N3CCN(C(=O)Nc4ccccc4)C4(CC4)C3)cc21] | 7-(3-(2,4-Difluoro-3-hydroxy-5- (trifluoromethyl)phenyl)-1-methyl-1H- pyrazolo[4,3-c]pyridin-6-yl)-N-phenyl-4,7- diazaspiro[2.5]octane-4-carboxamide |
| 22 | 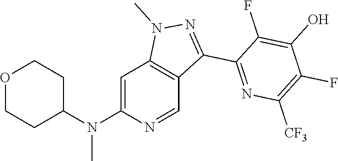 [SMILES: CN(c1cc2c(cn1)c(-c1nc(C(F)(F)F)c(F)c(O)c1F)nn2C)C1CCOCC1] | 3,5-Difluoro-2-(1-methyl-6-(methyl(tetrahydro- 2H-pyran-4-yl)amino)-1H-pyrazolo[4,3- c]pyridin-3-yl)-6-(trifluoromethyl)pyridin-4-ol |
| 23 | 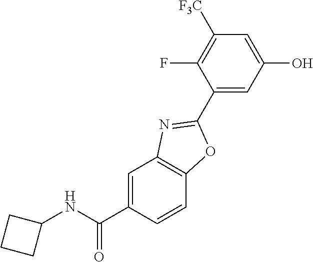 [SMILES: O=C(NC1CCC1)c1ccc2oc(-c3cc(O)cc(C(F)(F)F)c3F)nc2c1] | N-Cyclobutyl-2-(2-fluoro-5-hydroxy-3- (trifluoromethyl)phenyl)benzo[d]oxazole-5- carboxamide |
| 23.01 | 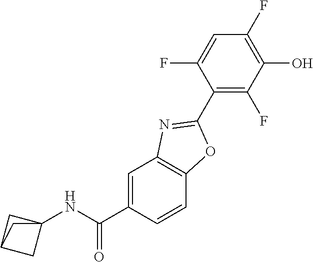 [SMILES: O=C(NC12CC(C1)C2)c1ccc2oc(-c3c(F)cc(F)c(O)c3F)nc2c1] | N-(Bicyclo[1.1.1]pentan-1-yl)-2-(2,4,6- trifluoro-3-hydroxyphenyl)benzo[d]oxazole-5- carboxamide |

In some embodiments, provided herein is a pharmaceutically acceptable salt or solvate of a compound that is described in Table 1.

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal fluids than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid to provide a “pharmaceutically acceptable acid addition salt.” In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with abase to provide a “pharmaceutically acceptable base addition salt.”

In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), crystalline forms (also known as polymorphs), or pharmaceutically acceptable salts of compounds described herein, as well as active metabolites of these compounds having the same type of activity.

In some embodiments, sites on the organic groups (e.g., alkyl groups, aromatic rings) of compounds described herein are susceptible to various metabolic reactions. Incorporation of appropriate substituents on the organic groups will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g., with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogen atoms of the compounds described herein is replaced with deuterium.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. The prodrug may be a substrate for a transporter. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically, or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound described herein as set forth herein are included within the scope of the claims. In some cases, some of the herein-described compounds is a prodrug for another derivative or active compound. In some embodiments, a prodrug of the compound disclosed herein permits targeted delivery of the compound to a particular region of the gastrointestinal tract. Formation of a pharmacologically active metabolite by the colonic metabolism of drugs is a commonly used “prodrug” approach for the colon-specific drug delivery systems.

In some embodiments, a prodrug is formed by the formation of a covalent linkage between drug and a carrier in such a manner that upon oral administration the moiety remains intact in the stomach and small intestine. This approach involves the formation of a prodrug, which is a pharmacologically inactive derivative of a parent drug molecule that requires spontaneous or enzymatic transformation in the biological environment to release the active drug. Formation of prodrugs has improved delivery properties over the parent drug molecule. The problem of stability of certain drugs from the adverse environment of the upper gastrointestinal tract can be eliminated by prodrug formation, which is converted into the parent drug molecule once it reaches the colon. Site specific drug delivery through site specific prodrug activation may be accomplished by the utilization of some specific property at the target site, such as altered pH or high activity of certain enzymes relative to the non-target tissues for the prodrug-drug conversion.

In some embodiments, covalent linkage of the drug with a carrier forms a conjugate. Such conjugates include, but are not limited to, azo bond conjugates, glycoside conjugates, glucuronide conjugates, cyclodextrin conjugates, dextran conjugates or amino-acid conjugates.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

In additional or further embodiments, the compounds are rapidly metabolized in plasma.

In additional or further embodiments, the compounds are rapidly metabolized by the intestines.

In additional or further embodiments, the compounds are rapidly metabolized by the liver.

### Synthesis of Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

The compounds described herein are prepared by the general synthetic routes described below in Schemes 1 to 7.

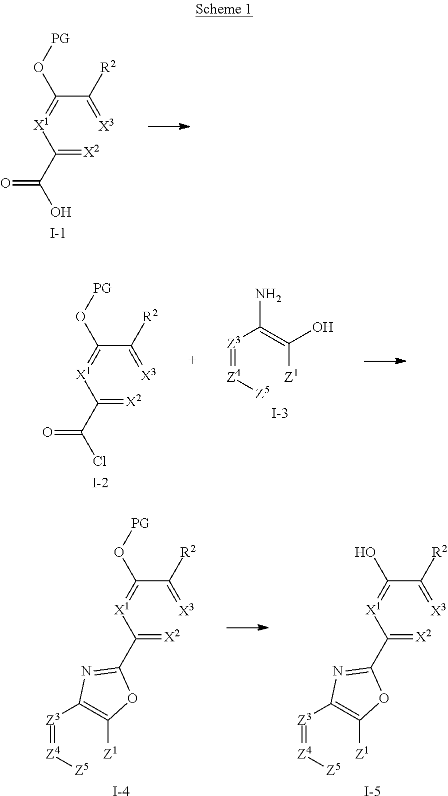

In some embodiments, compounds described herein are prepared as outlined in Scheme 1.

In some embodiments, carboxylic acid intermediate I-1 is reacted under suitable conditions to provide intermediate I-2. In some embodiments, suitable conditions include using appropriate reagents in an appropriate solvent or solvent mixture at an appropriate temperature and an appropriate amount of time. In some embodiments, the appropriate reagents are oxalyl chloride and DMF. In some embodiments, the appropriate solvent is a chlorinated solvent such as dichloromethane. In some embodiments, the suitable temperature is room temperature and the appropriate amount of time is about 1 hour.

In some embodiments, acyl chloride I-2 is reacted with a suitable intermediate I-3 under appropriate cyclization conditions to give benzoxazole I-4. In some embodiments, suitable cyclization conditions include but are not limited to the use of an appropriate acid in an appropriate solvent or solvent mixture at an appropriate temperature and an appropriate amount of time. In some embodiments, the appropriate acid is an organic acid such as methanesulfonic acid. In some embodiments, the appropriate solvent is dioxane. In some embodiments, the appropriate time and appropriate temperature is about 15 hours (overnight) at about 100° C.

In some embodiments, I-4 is reacted under suitable conditions to remove the phenol protecting group to provide I-5. In some embodiments, the appropriate protecting group is a benzyl protecting group. In some embodiments, appropriate conditions to remove a benzyl protecting group include using hydrogenation conditions using a suitable catalyst in a suitable solvent at an appropriate temperature and amount of time. In some embodiments, the appropriate catalyst is palladium on carbon. In some embodiments, the appropriate solvent is THF. In some embodiments, the suitable temperature is room temperature and the appropriate amount of time stirred under a hydrogen atmosphere at a suitable pressure is about 30 minutes. In some embodiments, the suitable pressure of hydrogen is atmospheric pressure.

In some embodiments, appropriate conditions to remove a methyl protecting group include using a suitable reagent in a suitable solvent at an appropriate temperature and amount of time. In some embodiments, the appropriate reagent is boron tribromide. In some embodiments, the appropriate solvent is a chlorinated solvent such as dichloromethane. In some embodiments, the suitable temperature is 0° C. to room temperature and the appropriate amount of time is about 3 hours.

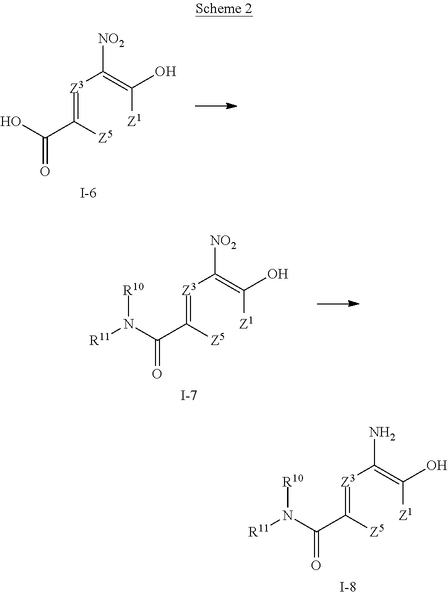

In some embodiments, compounds described herein are prepared as outlined in Scheme 2.

In some embodiments, carboxylic acid I-6 is reacted under suitable amide coupling conditions to provide amide I-7. In some embodiments, appropriate amide coupling conditions include using an appropriate coupling reagent and a suitable amine with an appropriate base and solvent at an appropriate time and at an appropriate temperature. In some embodiments, the appropriate coupling reagent is HATU. In some embodiments, the appropriate base is diisopropylethylamine. In some embodiments, the appropriate solvent is DMF. In some embodiments, the reaction temperature is about room temperature and the reaction time is about 15 hours (overnight).

In some embodiments, I-7 is reacted under suitable reduction conditions to provide I-8. In some embodiments, appropriate conditions include hydrogenation conditions using a suitable catalyst in a suitable solvent at an appropriate temperature and amount of time. In some embodiments, the appropriate catalyst is palladium on carbon. In some embodiments, the appropriate solvent is THF. In some embodiments, the suitable temperature is room temperature and the appropriate amount of time stirred under a hydrogen atmosphere at a suitable pressure is about 4 hours. In some embodiments, the suitable pressure of hydrogen is atmospheric pressure.

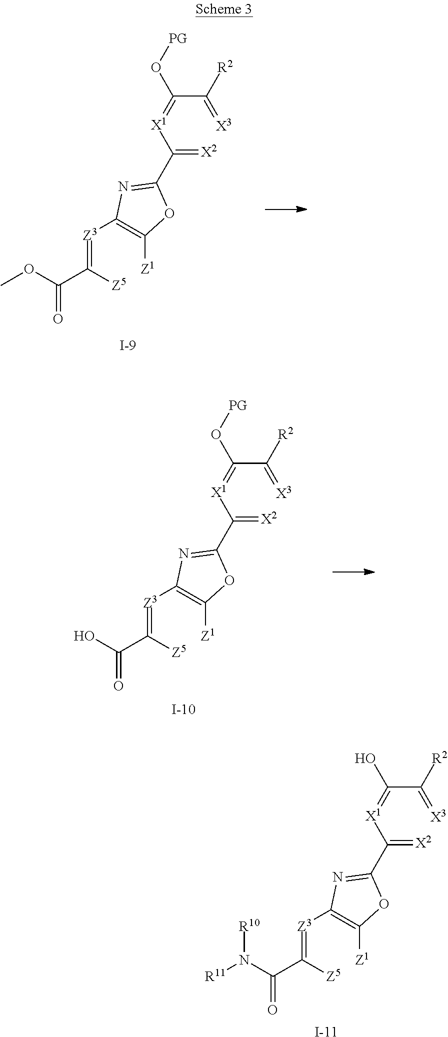

In some embodiments, compounds described herein are prepared as outlined in Scheme 3.

In some embodiments, carboxylic ester I-9 is reacted under suitable hydrolysis conditions to provide intermediate I-10. In some embodiments, suitable hydrolysis conditions include using an appropriate reagent in an appropriate solvent or solvent mixture at an appropriate temperature and an appropriate amount of time. In some embodiments, the appropriate reagent is sodium hydroxide. In some embodiments, the appropriate solvent mixture is THF:methanol:water. In some embodiments, the suitable temperature is room temperature and the appropriate amount of time is about 2 hours.

In some embodiments, carboxylic acid I-10 is reacted under suitable amide coupling conditions followed by removal of a suitable phenol protecting group to provide compound I-11. In some embodiments, appropriate amide coupling conditions include using an appropriate coupling reagent and a suitable amine with an appropriate base and solvent at an appropriate time and at an appropriate temperature. In some embodiments, the appropriate coupling reagent is HATU. In some embodiments, the appropriate base is diisopropylethylamine. In some embodiments, the appropriate solvent is DMF. In some embodiments, the reaction temperature is about room temperature and the reaction time is about 15 hours (overnight).

In some embodiments, a suitable protecting group is a benzyl protecting group. In some embodiments, appropriate conditions to remove a benzyl protecting group include using hydrogenation conditions using a suitable catalyst in a suitable solvent at an appropriate temperature and amount of time. In some embodiments, the appropriate catalyst is palladium on carbon. In some embodiments, the appropriate solvent is THF. In some embodiments, the suitable temperature is room temperature and the appropriate amount of time stirred under a hydrogen atmosphere at a suitable pressure is about 30 minutes. In some embodiments, the suitable pressure of hydrogen is atmospheric pressure.

In some embodiments, a suitable protecting group is a methyl protecting group. In some embodiments, appropriate conditions to remove a methyl protecting group include using a suitable reagent in a suitable solvent at an appropriate temperature and amount of time. In some embodiments, the appropriate reagent is boron tribromide. In some embodiments, the appropriate solvent is a chlorinated solvent such as dichloromethane. In some embodiments, the suitable temperature is 0° C. to room temperature and the appropriate amount of time is about 3 hours.

In some embodiments, the phenol protection group of intermediate I-10 is removed prior to amide formation to provide compound I-11.

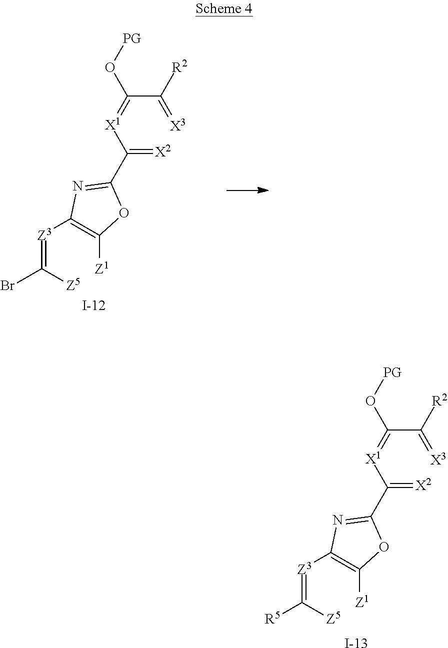

In some embodiments, compounds described herein are prepared as outlined in Scheme 4.

In some embodiments, where R⁵ is an aryl or heteroaryl ring system intermediate I-12 is reacted under appropriate Suzuki coupling reaction conditions using a suitable boronic acid or boronic ester and a suitable catalyst and appropriate base in a suitable solvent or solvent mixture at an appropriate temperature and amount of time to provide intermediate I-13. In some embodiments, the appropriate catalyst is tetrakis(triphenylphosphine)palladium(0). In some embodiments, the appropriate base is sodium carbonate. In some embodiments, the appropriate solvent mixture is dioxane:water. In some embodiments, the suitable temperature is 80° C. and the appropriate amount of time stirred is about 1 hour.

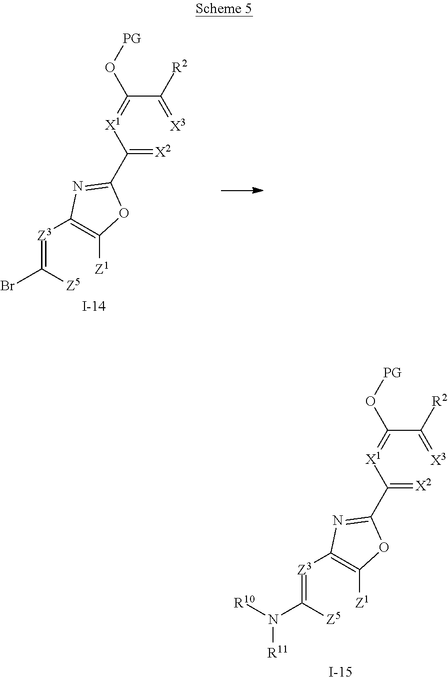

In some embodiments, compounds described herein are prepared as outlined in Scheme 5.

In some embodiments, intermediate I-14 is reacted with a suitable amine under appropriate Buchwald coupling reaction conditions using a suitable catalyst and catalyst ligand and a suitable base in a suitable solvent or solvent mixture at an appropriate temperature and amount of time. In some embodiments, the appropriate catalyst is tris(dibenzylideneacetone)dipalladium (0). In some embodiments, the appropriate catalyst ligand is RuPhos. In some embodiments, the appropriate base is sodium tert-butoxide. In some embodiments, the appropriate solvent is toluene or dioxane. In some embodiments, the suitable temperature is 100° C. and the appropriate amount of time stirred is about 15 hours (overnight).

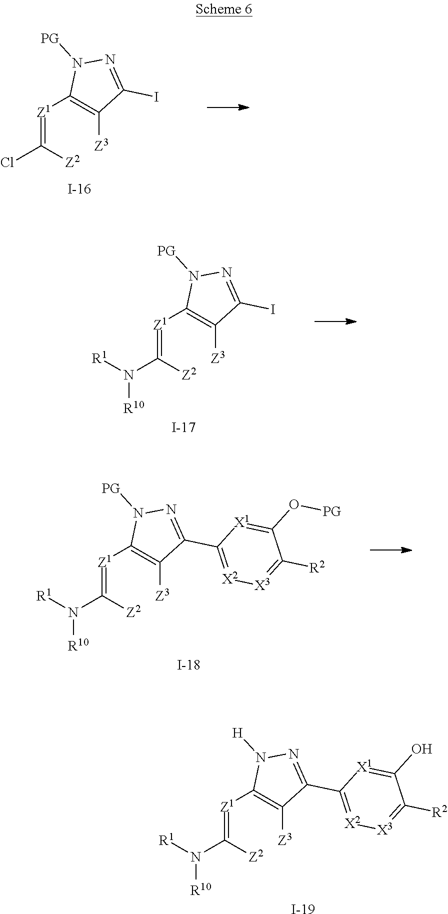

In some embodiments, compounds described herein are prepared as outlined in Scheme 6.

In some embodiments, intermediate I-16 is reacted under appropriate conditions with a suitable amine and a suitable base using a suitable solvent or solvent mixture at an appropriate temperature and amount of time to give intermediate I-17. In some embodiments, a suitable base is Hunig's base. In some embodiments, a suitable solvent is DMA. In some embodiments, a suitable temperature is 100° C. and a suitable time is 1 h.

In some embodiments, intermediate I-17 is reacted under appropriate Suzuki coupling reaction conditions with a suitable aryl-halide using a suitable catalyst and a suitable base in a suitable solvent or solvent mixture at an appropriate temperature and amount of time to provide intermediate I-17. In some embodiments, the appropriate catalyst is tetrakis(triphenylphosphine)palladium(0). In some embodiments, the appropriate base is sodium carbonate. In some embodiments, the appropriate solvent mixture is dioxane:water. In some embodiments, the suitable temperature is 80° C. and the appropriate amount of time is about 1 hour.

In some embodiments, intermediate I-18 is reacted under suitable phenol deprotection conditions to provide I-19. In some embodiments, the protecting group is a benzyl protecting group. In some embodiments, appropriate conditions to remove a benzyl protecting group include using hydrogenation conditions using a suitable catalyst in a suitable solvent at an appropriate temperature and amount of time. In some embodiments, the appropriate catalyst is palladium on carbon. In some embodiments, the appropriate solvent is THF. In some embodiments, the suitable temperature is room temperature and the appropriate amount of time stirred under a hydrogen atmosphere at a suitable pressure is about 30 minutes. In some embodiments, the suitable pressure of hydrogen is atmospheric pressure.

In some embodiments the protecting group is a methyl protecting group. In some embodiments, appropriate conditions to remove a methyl protecting group include using a suitable reagent in a suitable solvent at an appropriate temperature and amount of time. In some embodiments, the appropriate reagent is boron tribromide. In some embodiments, the appropriate solvent is a chlorinated solvent such as dichloromethane. In some embodiments, the suitable temperature is 0° C. to room temperature and the appropriate amount of time is about 3 hours.

In some embodiments the protecting group is a MOM-protecting group. In some embodiments, appropriate conditions to remove a MOM-protecting group include using a suitable acid in a suitable solvent at an appropriate temperature and amount of time. In some embodiments, the appropriate acid is trifluoroacetic acid. In some embodiments, the appropriate solvent is a chlorinated solvent such as dichloromethane. In some embodiments, the suitable temperature is room temperature and the appropriate amount of time is 1 to 15 hours (overnight).

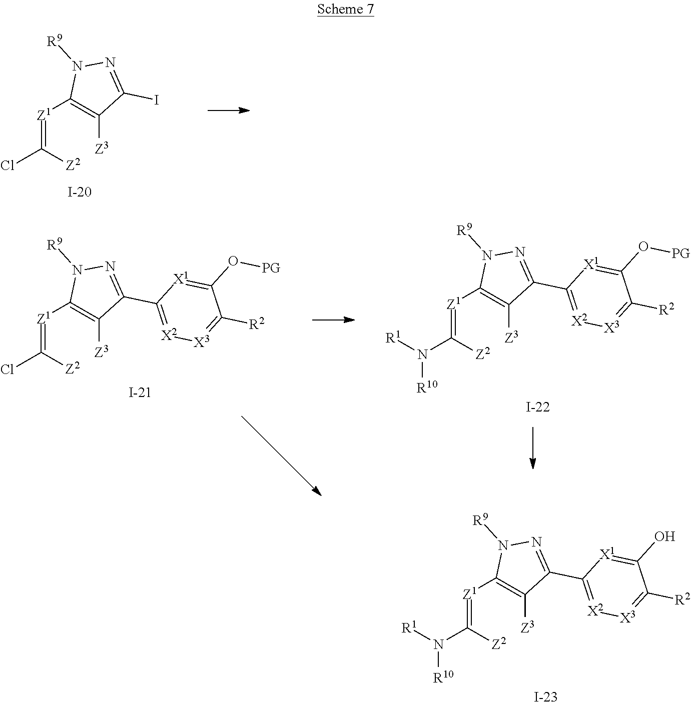

In some embodiments, compounds described herein are prepared as outlined in Scheme 7.

In some embodiments, intermediate I-20 is reacted under appropriate Suzuki coupling reaction conditions using a suitable aryl-halide and suitable catalyst and base in a suitable solvent or solvent mixture at an appropriate temperature and amount of time to provide intermediate I-21. In some embodiments, the appropriate catalyst is 1,1′-bis(diphenylphosphino)ferrocene dichloropalladium (II). In some embodiments, the appropriate base is potassium fluoride. In some embodiments, the appropriate solvent mixture is dioxane:water. In some embodiments, the suitable temperature is 90° C. and the appropriate amount of time stirred is about 30 minutes.

In some embodiments, intermediate I-21 is reacted under appropriate Buchwald coupling reaction conditions using an appropriate amine and a suitable catalyst and catalyst ligand and a suitable base in a suitable solvent or solvent mixture at an appropriate temperature and amount of time to give intermediate I-22. In some embodiments, the appropriate catalyst is tris(dibenzylideneacetone)dipalladium (0). In some embodiments, the appropriate catalyst ligand is RuPhos. In some embodiments, the appropriate base is sodium tert-butoxide. In some embodiments, the appropriate solvent is dioxane. In some embodiments, the suitable temperature is 90° C. and the appropriate amount of time stirred is about 60 minutes to 15 hours (overnight).

In some embodiments, intermediate I-22 is deprotected to provide I-23. In some embodiments, the protecting group is a MOM-protecting group. In some embodiments, appropriate conditions to remove a MOM-protecting group include using a suitable acid in a suitable solvent at an appropriate temperature and amount of time. In some embodiments, the appropriate acid is trifluoroacetic acid. In some embodiments, the appropriate solvent is a chlorinated solvent such as dichloromethane. In some embodiments, the suitable temperature is room temperature and the appropriate amount of time is 15 min to 15 hours (overnight).

In some embodiments, intermediate I-21 is reacted under appropriate coupling conditions using an appropriate amine and a suitable base in a suitable solvent or solvent mixture at an appropriate temperature and amount of time and is also deprotected to provide I-23. In some embodiments, the protecting group is a MOM-protecting group. In some embodiments, the appropriate base is DIEA. In some embodiments, the appropriate solvent is dimethylacetamide. In some embodiments, the appropriate solvent is NMP. In some embodiments, the suitable temperature is 100° C.-150° C. and the appropriate amount of time is about 1 hour.

In some embodiments, compounds are prepared as described in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, 4 carbon atoms, 5 carbon atoms, 6 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl group. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. In certain embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises two carbon atoms (e.g., C₂ alkylene). In other embodiments, an alkylene comprises two to four carbon atoms (e.g., C₂-C₄ alkylene). Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized π-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon or nitrogen atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic group, wherein each of the atoms forming the ring (i.e. skeletal atoms) is a carbon atom. In some embodiments, cycloalkyls are spirocyclic or bridged compounds. In some embodiments, cycloalkyls are fully saturated. In some embodiments, cycloalkyls are partially unsaturated. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a monocyclic cycloalkyl. Monocyclic cycloalkyls include, but are not limited to, cyclopropyl, cyclobutyl, cyclopentyl, cyclohexyl, cycloheptyl, and cyclooctyl. Poly cyclic cycloalkyls include, for example, adamantyl, norbornyl (i.e., bicyclo[2.2.1]heptanyl), norbornenyl, decalinyl, 7,7-dimethyl-bicyclo[2.2.1]heptanyl, and the like

The term “halo” or, alternatively, “halogen” or “halide” means fluoro, chloro, bromo or iodo. In some embodiments, halo is fluoro, chloro, or bromo.

The term “haloalkyl” refers to an alkyl in which one or more hydrogen atoms are replaced by a halogen atom. In one aspect, a fluoroalkyl is a C₁-C₆fluoroalkyl.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g., —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heteroalkylene” refers to a divalent heteroalkyl group.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, benzotriazole, purine, quinolizine, quinoline, isoquinoline, cinnoline, phthalazine, quinazoline, quinoxaline, 1,8-naphthyridine, and pteridine. In some embodiments, a heteroaryl contains 0-4N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, heterocycloalkyls are spirocyclic or bridged compounds. In some embodiments, heterocycloalkyls are fully saturated. In some embodiments, heterocycloalkyls are partially unsaturated. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2N atoms in the ring. In some embodiments, a heterocycloalkyl contains 0-2N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s). In some other embodiments, optional substituents are individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, —CH₂CO₂H, —CH₂CO₂alkyl, —CH₂C(═O)NH₂, —CH₂C(═O)NH(alkyl), —CH₂C(═O)N(alkyl)₂, —CH₂S(═O)₂NH₂, —CH₂S(═O)₂NH(alkyl), —CH₂S(═O)₂N(alkyl)₂, alkyl, alkenyl, alkynyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, substituted groups are substituted with one of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In some embodiments, a compound disclosed herein is formulated to provide a controlled release of the compound. Controlled release refers to the release of the compound described herein from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

Approaches to deliver the intact therapeutic compound to the particular regions of the gastrointestinal tract (e.g., such as the colon), include:
(i) Coating with polymers: The intact molecule can be delivered to the colon without absorbing at the upper part of the intestine by coating of the drug molecule with the suitable polymers, which degrade only in the colon.
(ii) Coating with pH-sensitive polymers: The majority of enteric and colon targeted delivery systems are based on the coating of tablets or pellets, which are filled into conventional hard gelatin capsules. Most commonly used pH-dependent coating polymers are methacrylic acid copolymers, commonly known as Eudragit® S, more specifically Eudragit® L and Eudragit® S. Eudragit® L100 and S 100 are copolymers of methacrylic acid and methyl methacrylate. Additional pH-dependent coating polymers include cellulose acetate phthalate (CAP), hydroxypropyl methylcellulose phthalate (HPMCP), polyvinyl acetate phthalate (PVAP) and cellulose acetate trimelliate.
(iii) Coating with biodegradable polymers;
(iv) Embedding in matrices;
(v) Embedding in biodegradable matrices and hydrogels;
(vi) Embedding in pH-sensitive matrices;
(vii) Timed release systems;
(viii) Redox-sensitive polymers;
(ix) Bioadhesive systems;
(x) Coating with microparticles;
(xi) Osmotic controlled drug delivery.

Another approach towards colon-targeted drug delivery or controlled-release systems includes embedding the drug in polymer matrices to trap it and release it in the colon. These matrices can be pH-sensitive or biodegradable. Matrix-Based Systems, such as multi-matrix (MMX)-based delayed-release tablets, ensure the drug release in the colon.

Additional pharmaceutical approaches to targeted delivery of therapeutics to particular regions of the gastrointestinal tract are known. Chourasia M K, Jain S K, Pharmaceutical approaches to colon targeted drug delivery systems. J Pharm Sci. 2003 January-April; 6(1):33-66. Patel M, Shah T, Amin A. Therapeutic opportunities in colon-specific drug-delivery systems Crit Rev Ther Drug Carrier Syst. 2007; 24(2):147-202. Kumar P, Mishra B. Colon targeted drug delivery systems—an overview. Curr Drug Deliv. 2008 July; 5(3):186-98. Van den Mooter G. Colon drug delivery. Expert Opin Drug Deliv. 2006 January; 3(1):111-25. Seth Amidon, Jack E. Brown, and Vivek S. Dave, Colon-Targeted Oral Drug Delivery Systems: Design Trends and Approaches, AAPS Pharm Sci Tech. 2015 August; 16(4): 731-741.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds described herein, or a pharmaceutically acceptable salt thereof, are used in the preparation of medicaments for the treatment of diseases or conditions in a mammal that would benefit from administration of an HSD17B13 inhibitor. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In some embodiments, described herein is a method of treating or preventing a liver disease or condition in a mammal, comprising administering to the mammal a compound of Formula (I″), (I′), (I), (Ia′), (II″), (II′), (II), (IIa′), or (IIb′), or a pharmaceutically acceptable salt or solvate thereof. In some embodiments, described herein is a method of treating or preventing an alcoholic or nonalcoholic liver disease or condition in a mammal, comprising administering to the mammal a compound of Formula (I″), (I′), (I), (Ia′), (II″), (II′), (II), (IIa′), or (IIb′), or a pharmaceutically acceptable salt or solvate thereof. In some embodiments, the liver disease or condition is an alcoholic liver disease or condition. In some embodiments, the liver disease or condition is a nonalcoholic liver disease or condition. In some embodiments, the liver disease or condition is liver inflammation, fatty liver (steatosis), liver fibrosis, hepatitis, cirrhosis, hepatocellular carcinoma, or combinations thereof. In some embodiments, the liver disease or condition is primary biliary cirrhosis, primary sclerosing cholangitis, cholestasis, nonalcoholic steatohepatitis (NASH), nonalcoholic fatty liver disease (NAFLD), or combinations thereof. In some embodiments, the liver disease or condition described herein is a chronic liver disease or condition.

In some embodiments, described herein is a method of modulating HSD17B13 activity in a mammal, comprising administering to the mammal a compound of Formula (I″), (I′), (I), (Ia′), (II″), (II′), (II), (IIa′), or (IIb′), or a pharmaceutically acceptable salt or solvate thereof. In some embodiments, modulating comprises inhibiting HSD17B13 activity. In some embodiments of a method of modulating HSD17B13 activity in a mammal, the mammal has a liver disease or condition selected from liver inflammation, fatty liver (steatosis), liver fibrosis, hepatitis, cirrhosis, hepatocellular carcinoma, and combinations thereof. In some embodiments of a method of modulating HSD17B13 activity in a mammal, the mammal has a liver disease or condition selected from primary biliary cirrhosis, primary sclerosing cholangitis, cholestasis, nonalcoholic steatohepatitis (NASH), nonalcoholic fatty liver disease (NAFLD), and combinations thereof.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder, or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion, the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder, or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

It is understood that the dosage regimen to treat, prevent, or ameliorate the condition(s) for which relief is sought, is modified in accordance with a variety of factors (e.g., the disease, disorder, or condition from which the subject suffers; the age, weight, sex, diet, and medical condition of the subject). Thus, in some instances, the dosage regimen actually employed varies and, in some embodiments, deviates from the dosage regimens set forth herein.

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

### EXAMPLES

The following examples are provided for illustrative purposes only and not to limit the scope of the claims provided herein.

As used above, and throughout the description of the invention, the following abbreviations, unless otherwise indicated, shall be understood to have the following meanings:

| | |
|---|---|
| acac | acetylacetone |
| ACN or MeCN | acetonitrile |
| AcOH | acetic acid |
| Ac | acetyl |
| i-AmOH | isoamyl alcohol or 3-methyl-1-butanol |
| t-AmOH | tert-Amyl alcohol or 2-methyl-2-butanol |
| BINAP | 2,2′-bis(diphenylphosphino)-1,1′-binaphthalene |
| Bn | benzyl |
| BOC or Boc | tert-butyl carbamate |
| i-Bu | iso-butyl |
| t-Bu | tert-butyl |
| Cy | cyclohexyl |
| CDI | 1,1-carbonyldiimidazole |
| CPME | cyclopentyl methyl ether |
| DBA or dba | dibenzylideneacetone |
| DCE | dichloroethane (ClCH₂CH₂Cl) |
| DCM | dichloromethane (CH₂Cl₂) |
| DHP | 3,4-dihydropyran |
| DIBAL-H | diisobutylaluminum hydride |
| DIPEA or DIEA | diisopropylethylamine |
| DMAP | 4-(N,N-dimethylamino)pyridine |
| DME | 1,2-dimethoxyethane |
| DMF | N,N-dimethylformamide |
| DMA | N,N-dimethylacetamide |
| DMPU | N,N′-dimethylpropyleneurea |
| DMSO | dimethylsulfoxide |
| Dppf or dppf | 1,1′-bis(diphenylphosphino)ferrocene |
| EDC or EDCI | N-(3-dimethylaminopropyl)-N′-ethylcarbodiimide |
| | hydrochloride |
| EEDQ | 2-Ethoxy-1-ethoxycarbonyl-1,2-dihydroquinoline |
| eq | equivalent(s) |
| Et | ethyl |
| Et₂O | diethyl ether |
| EtOH | ethanol |
| EtOAc | ethyl acetate |
| HATU | 1-[bis(dimethylamino)methylene]-1H-1,2,3- |
| | triazolo[4,5-b ]pyridinium 3-oxid hexafluorophosphate |
| HMPA | hexamethylphosphoramide |
| HOBt | 1-hydroxybenzotriazole |
| HPLC | high performance liquid chromatography |
| IBX | 2-iodoxybenzoic acid |
| KOAc | potassium acetate |
| KHMDS | potassium bis(trimethylsilyl)amide |
| NaHMDS | sodium bis(trimethylsilyl)amide |
| LiHMDS | lithium bis(trimethylsilyl)amide |
| LAH | lithium aluminum anhydride |
| LCMS | liquid chromatography mass spectrometry |
| 2-MeTHF | 2-methyltetrahydrofuran |
| Me | methyl |
| MeOH | methanol |
| MOM | methoxymethyl ether |
| MS | mass spectroscopy |
| Ms | mesyl |
| MTBE | methyl tert-butyl ether |
| NBS | N-bromosuccinimide |
| NCS | N-chlorosuccinimide |
| NIS | N-iodosuccinimide |
| NMM | N-methyl-morpholine |
| NMP | N-methy-pyrrolidin-2-one |
| NMR | nuclear magnetic resonance |
| OTf | trifluoromethanesulfonate |
| PCC | pyridinium chlorochromate |
| PE | petroleum ether |
| PG | protecting group |
| Ph | phenyl |
| PPTS | pyridium p-toluenesulfonate |
| iPr/i-Pr | iso-propyl |
| RP-HPLC | reverse-phase high-pressure liquid chromatography |
| rt | room temperature |
| TBS | tert-butyldimethylsilyl |
| TBAF | tetra-n-butylammonium fluoride |
| TBAI | tetra-n-butylammonium iodide |
| TEA | triethylamine |
| TFA | trifluoroacetic acid |
| THF | tetrahydrofuran |
| TLC | thin layer chromatography |
| TMEDA | N,N,N′,N′-tetramethylethylenediamine |
| TMS | trimethylsilyl |
| TsOH/p-TsOH | p-toluenesulfonic acid |

### Intermediate 1

### Methyl 2-(3-(benzyloxy)-4,5-difluorophenyl)benzo[d]oxazole-5-carboxylate

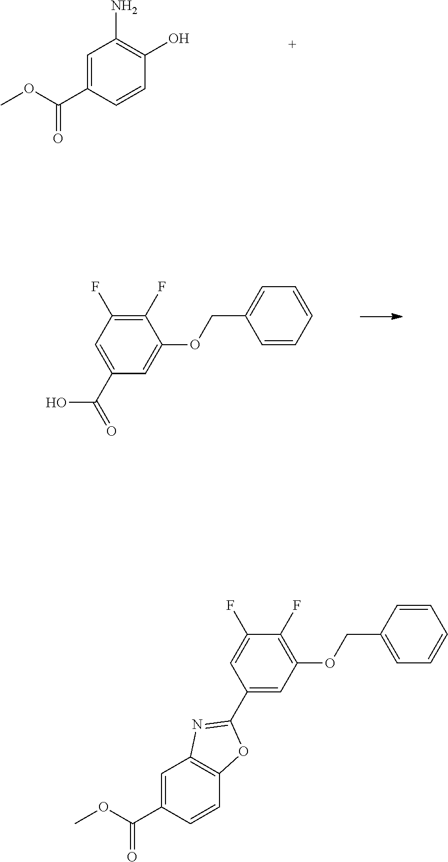 [SMILES: COC(=O)c1ccc(O)c(N)c1 ; O=C(O)c1cc(F)c(F)c(OCc2ccccc2)c1 ; COC(=O)c1ccc2oc(-c3cc(F)c(F)c(OCc4ccccc4)c3)nc2c1]

Dimethylformamide (0.05 mL) was added to a suspension of 4,5-difluoro-3-(phenylmethoxy)benzoic acid (1.29 g, 4.90 mmol) and oxalyl chloride (0.65 mL, 7.58 mmol) in DCM (20 mL) at rt. The reaction was stirred for 80 min, concentrated, dissolved in DCM (30 mL), re-concentrated, dried under vacuum, and then dissolved in dioxane (20 mL). The solution was added to a solution of methyl 3-amino-4-hydroxybenzoate (730 mg, 4.37 mmol) and dioxane (20 mL). After stirring the mixture for 75 min, methanesulfonic acid (1.6 mL, 24.5 mmol) was added to the reaction. The mixture was heated at 100° C. for 18 h, cooled to rt, diluted (100 mL EtOAc), washed (75 mL saturated NaHCO₃ and then 75 mL brine), dried (Na₂SO₄), and then concentrated. The residue was triturated with acetonitrile (30 mL) to give methyl 2-(3-(benzyloxy)-4,5-difluorophenyl)benzo[d]oxazole-5-carboxylate (800 mg, 410). The purification of the filtrate by silica gel chromatography (0-15% EtOAc/hexanes) gave an additional 75 mg of material. ¹H NMR (400 MHz, DMSO-d₆): δ 8.36-8.32 (m, 1H), 8.12-8.07 (m, 1H), 7.96-7.88 (m, 2H), 7.85 (ddd, J=1.8, 6.7, 10.2 Hz, 1H), 7.58-7.51 (m, 2H), 7.49-7.36 (m, 3H), 5.41 (s, 2H), 3.93-3.89 (m, 3H); LCMS 396.0 [M+H]⁺.

The Intermediates below were synthesized in a similar manner to that described for Intermediate 1.

| Int | Structure | Name | [M + H]⁺ |
|---|---|---|---|
| 1.01 | 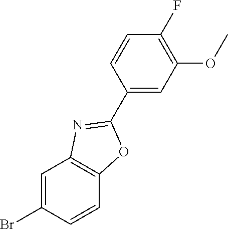 [SMILES: COc1cc(-c2nc3cc(Br)ccc3o2)ccc1F] | 5-Bromo-2-(4-fluoro-3- methoxyphenyl)benzo[d]oxazole | 321.9 |
| 1.02 | 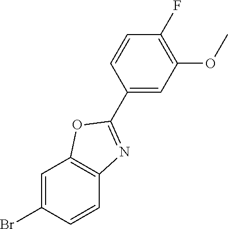 [SMILES: COc1cc(-c2nc3ccc(Br)cc3o2)ccc1F] | 6-Bromo-2-(4-fluoro-3- methoxyphenyl)benzo[d]oxazole | 321.8 |
| 1.03 | 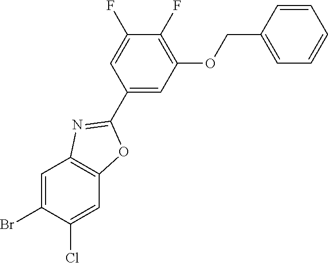 [SMILES: Fc1cc(-c2nc3cc(Br)c(Cl)cc3o2)cc(OCc2ccccc2)c1F] | 2-(3-(Benzyloxy)-4,5- difluorophenyl)-5-bromo-6- chlorobenzo[d]oxazole | 451.9 |
| 1.04 | 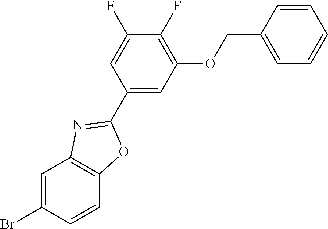 [SMILES: Fc1cc(-c2nc3cc(Br)ccc3o2)cc(OCc2ccccc2)c1F] | 2-(3-(Benzyloxy)-4,5- difluorophenyl)-5- bromobenzo[d]oxazole | 415.9 |

### Intermediate 2

### 3-Amino-N-cyclobutyl-4-hydroxybenzamide

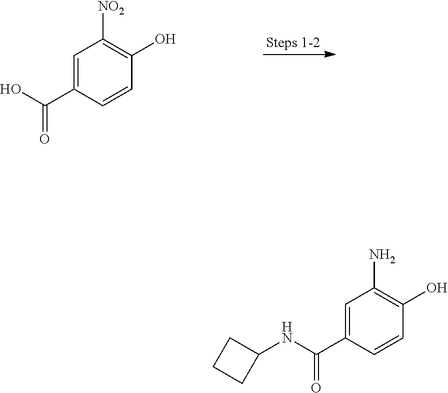 [SMILES: O=C(O)c1ccc(O)c([N+](=O)[O-])c1 ; Nc1cc(C(=O)NC2CCC2)ccc1O]

### Step 1: N-Cyclobutyl-4-hydroxy-3-nitrobenzamide

Diisopropylethylamine (5.80 mL, 33.3 mmol) was added to a mixture of 4-hydroxy-3-nitrobenzoic acid (3.02 g, 16.5 mmol) and HATU (7.51 g, 19.8 mmol) in DMF (45 mL) at rt. The mixture was stirred for 30 min. Cyclobutylamine (2.1 mL, 24.7 mmol) was added to the reaction. The mixture was stirred overnight and then diluted (100 mL EtOAc). The organic phase was washed (2×100 mL water and then 100 mL brine), dried (Na₂SO₄), and then concentrated. The residue was purified by silica gel chromatography (0-10% EtOAc/DCM) to give N-cyclobutyl-4-hydroxy-3-nitrobenzamide (2.3 g, 59%) as a yellow solid. ¹H NMR (400 MHz, DMSO-d₆): δ 11.57 (s, 1H), 8.71 (br d, J=7.5 Hz, 1H), 8.43 (d, J=2.2 Hz, 1H), 8.03 (dd, J=2.2, 8.7 Hz, 1H), 7.18 (d, J=8.7 Hz, 1H), 4.45-4.35 (m, 1H), 2.26-2.17 (m, 2H), 2.12-1.99 (m, 2H), 1.73-1.62 (m, 2H).

### Step 2: 3-Amino-N-cyclobutyl-4-hydroxybenzamide

A mixture of N-cyclobutyl(4-hydroxy-3-nitrophenyl)carboxamide (2.30 g, 9.74 mmol), 10% Pd/C (0.21 g), THF (40 mL) and ethanol (40 mL) was stirred under a balloon of hydrogen for 2 h then filtered. The filter cake was washed (20 mL THF), and the filtrate was concentrated. The residue was triturated (50 mL DCM) to give 3-amino-N-cyclobutyl-4-hydroxybenzamide (1.45 g, 72%) as a gray solid. ¹H NMR (400 MHz, DMSO-d₆): δ 9.82-9.15 (m, 1H), 8.17 (s, 1H), 7.10 (d, J=2.2 Hz, 1H), 6.96 (dd, J=2.1, 8.1 Hz, 1H), 6.64 (d, J=8.1 Hz, 1H), 5.01-4.45 (m, 2H), 4.43-4.27 (m, 1H), 2.24-2.10 (m, 2H), 2.10-1.94 (m, 2H), 1.72-1.59 (m, 2H); LCMS 206.9 [M+H]⁺.

The Intermediates below were synthesized in a similar manner to that described for Intermediate 2.

| Int | Structure | Name | [M + H]⁺ |
|---|---|---|---|
| 2.01 | 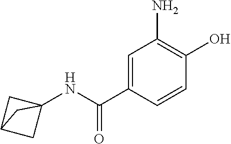 [SMILES: Nc1cc(C(=O)NC23CC(C2)C3)ccc1O] | 3-Amino-N-(bicyclo[1.1.1]pentan-1- yl)-4-hydroxybenzamide | 218.9 |

### Intermediate 3

### Methyl 2-(3-(benzyloxy)-4-fluoro-5-(trifluoromethyl)phenyl)benzo[d]oxazole-5-carboxylate

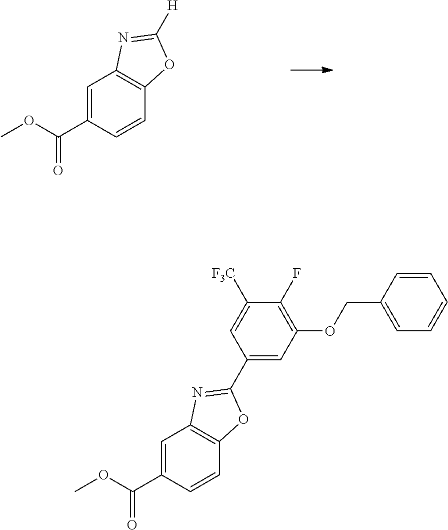 [SMILES: COC(=O)c1ccc2ocnc2c1 ; COC(=O)c1ccc2oc(-c3cc(OCc4ccccc4)c(F)c(C(F)(F)F)c3)nc2c1]

A mixture of methyl benzo[d]oxazole-5-carboxylate (300 mg, 1.69 mmol), 1-(benzyloxy)-5-bromo-2-fluoro-3-(trifluoromethyl)benzene (566 mg, 1.62 mmol), palladium acetate (40 mg, 0.18 mmol), copper(II) acetate (79 mg, 0.43 mmol), K₂CO₃ (477 mg, 3.45 mmol), PCy₃ (237 mg, 0.85 mmol), and toluene (4 mL) was heated at 160° C. for 15 min in a microwave. The reaction was diluted (100 mL), washed (75 mL water and then 75 mL brine), dried (Na₂SO₄), and then concentrated. The residue was purified by silica gel chromatography (0-30% EtOAc/hexanes) to give methyl 2-(3-(benzyloxy)-4-fluoro-5-(trifluoromethyl)phenyl)benzo[d]oxazole-5-carboxylate (343 mg, 45%) as a pale yellow solid. ¹H NMR (400 MHz, DMSO-d₆) δ: 8.38 (s, 1H), 8.35 (br d, J=7.2 Hz, 1H), 8.11 (d, J=8.3 Hz, 1H), 8.06 (br d, J=4.9 Hz, 1H), 7.98 (d, J=8.7 Hz, 1H), 7.57-7.53 (m, 2H), 7.50-7.43 (m, 2H), 7.41 (br d, J=7.0 Hz, 1H), 5.47 (s, 2H), 3.92 (s, 3H); LCMS 445.9 [M+H]⁺.

The Intermediates below were synthesized in a similar manner to that described for Intermediate 3.

| Int | Structure | Name | [M + H]⁺ |
|---|---|---|---|
| 3.01 | 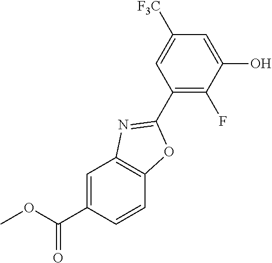 [SMILES: COC(=O)c1ccc2oc(-c3cc(C(F)(F)F)cc(O)c3F)nc2c1] | Methyl 2-(2-fluoro-3-hydroxy-5- (trifluoromethyl)phenyl)benzo[d]oxa- zole-5-carboxylate | 355.9 |
| 3.02 | 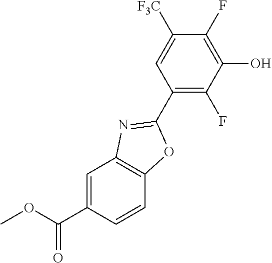 [SMILES: COC(=O)c1ccc2oc(-c3cc(C(F)(F)F)c(F)c(O)c3F)nc2c1] | Methyl 2-(2,4-difluoro-3-hydroxy-5- (trifluoromethyl)phenyl)benzo[d]oxa- zole-5-carboxylate | 373.9 |
| 3.03 | 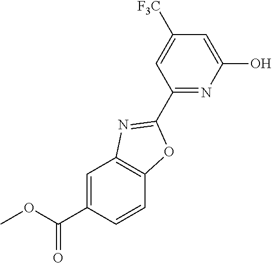 [SMILES: COC(=O)c1ccc2oc(-c3cc(C(F)(F)F)cc(O)n3)nc2c1] | Methyl 2-(6-hydroxy-4- (trifluoromethyl)pyridin-2- yl)benzo[d]oxazole-5-carboxylate | 339.0 |

### Intermediate 4

### 4-Fluoro-3-(methoxymethoxy)-5-(trifluoromethyl)benzoic Acid

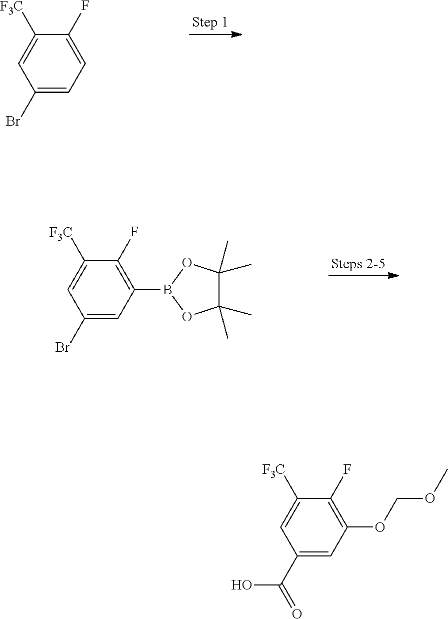 [SMILES: Fc1ccc(Br)cc1C(F)(F)F ; CC1(C)OB(c2cc(Br)cc(C(F)(F)F)c2F)OC1(C)C ; COCOc1cc(C(=O)O)cc(C(F)(F)F)c1F]

### Step 1: 2-(5-Bromo-2-fluoro-3-(trifluoromethyl)phenyl)-4,4,5,5-tetramethyl-1,3,2-dioxaborolane

A mixture of (1,5-cyclooctadiene)(methoxy)iridium(I) dimer (273 mg, 0.411 mmol) and bis(pinacolatobiboron) (2.87 g, 11.3 mmol) in THF (30 mL) was stirred until a clear yellow solution was obtained. A solution of 4,4′-di-tert-butyl-2,2′-bipyridine (110 mg, 0.41 mmol) in THF (30 mL) was added to the mixture, and the reaction was stirred until the reaction became a dark brown solution. 4-Bromo-1-fluoro-2-(trifluoromethyl)benzene (5.0 g, 20.6 mmol) was added to the mixture. The reaction was stirred overnight, slowly poured into H₂O (60 mL), and then extracted (3×50 mL EtOAc). The combined organic layers were washed (100 mL brine), dried (Na₂SO₄), filtered, and then concentrated. The residue was purified by silica gel chromatography (70% EtOAc/petroleum ether) to give 2-(5-bromo-2-fluoro-3-(trifluoromethyl)phenyl)-4,4,5,5-tetramethyl-1,3,2-dioxaborolane (3.0 g, 39%) as a red oil. ¹H NMR (400 MHz, DMSO-d₆): δ 8.13-8.11 (m, 1H), 7.98-7.96 (m, 1H), 1.31 (s, 12H).

### Step 2: 5-Bromo-2-fluoro-3-(trifluoromethyl)phenol

Hydrogen peroxide (18.4 g, 162 mmol) was slowly added to a solution of 2-(5-bromo-2-fluoro-3-(trifluoromethyl)phenyl)-4,4,5,5-tetramethyl-1,3,2-dioxaborolane (3.0 g, 8.13 mmol) in MeOH (20 mL). The mixture was stirred at rt for 2 h, quenched by slow addition of saturated Na₂SO₃ (70 mL), stirred for 0.5 h, and then extracted (3×50 mL EtOAc). The combined organic layers were washed (100 mL brine), dried (Na₂SO₄), filtered, and then concentrated. The residue was purified by silica gel chromatography (300% EtOAc/petroleum ether) to give 5-bromo-2-fluoro-3-(trifluoromethyl)phenol (1.8 g, 85%) as an oil. ¹H NMR (400 MHz, DMSO-d₆): δ 11.13 (s, 1H), 7.41-7.39 (m, 1H), 7.27-7.26 (m, 1H).

### Step 3: 5-Bromo-2-fluoro-1-(methoxymethoxy)-3-(trifluoromethyl)benzene

MOMCl (677 mg, 8.41 mmol) was added dropwise to a solution of 5-bromo-2-fluoro-3-(trifluoromethyl)phenol (1.8 g, 6.95 mmol) and DIPEA (1.35 g, 10.4 mmol) in DCM (20 mL) at 0° C. The mixture was stirred at rt overnight, slowly poured into H₂O (40 mL), and then extracted (3×30 mL EtOAc). The combined organic layers were washed (70 mL brine), dried (Na₂SO₄), filtered, and then concentrated. The residue was purified by silica gel chromatography (10% EtOAc/petroleum ether) to give 5-bromo-2-fluoro-1-(methoxymethoxy)-3-(trifluoromethyl)benzene (1.4 g, 66%) as a colorless oil. ¹H NMR (400 MHz, DMSO-d₆): δ 7.80 (d, 1H), 7.58 (d, 1H), 5.37 (s, 2H), 3.42 (s, 3H).

### Step 4: Methyl 4-fluoro-3-(methoxymethoxy)-5-(trifluoromethyl)benzoate

Pd(dppf)Cl₂ (338 mg, 0.461 mmol) was added to a mixture of 5-bromo-2-fluoro-1-(methoxymethoxy)-3-(trifluoromethyl)benzene (1.4 g, 4.62 mmol) and Et₃N (2.34 g, 23.1 mmol) in MeOH (20 mL). The suspension was degassed with 3 vacuum/CO cycles, stirred under CO (15 psi) at 70° C. overnight, and then concentrated. The residue was purified by silica gel chromatography (10% EtOAc/petroleum ether) to give methyl 4-fluoro-3-(methoxymethoxy)-5-(trifluoromethyl)benzoate (1.0 g, 76%) as a yellow oil. ¹H NMR (400 MHz, DMSO-d₆): δ 8.08 (d, 1H), 7.84 (d, 1H), 5.42 (s, 2H), 3.89 (s, 3H), 3.44 (s, 3H).

### Step 5: 4-Fluoro-3-(methoxymethoxy)-5-(trifluoromethyl)benzoic Acid

LiOH H₂O (2.97 g, 70.9 mmol) was added to a mixture of methyl 4-fluoro-3-(methoxymethoxy)-5-(trifluoromethyl)benzoate (1.0 g, 3.54 mmol), THF (20 mL), H₂O (10 mL), and MeOH (10 mL). The mixture was stirred at 50° C. for 2 h and concentrated to remove MeOH. Aqueous hydrochloric acid (1 M) was slowly added to the mixture to adjust the pH to 6, and the mixture was extracted (3×50 mL EtOAc). The combined organic layers were washed (100 mL brine), dried (Na₂SO₄), filtered, and then concentrated. The residue was purified by silica gel chromatography (10% EtOAc/petroleum ether) to give 4-fluoro-3-(methoxymethoxy)-5-(trifluoromethyl)benzoic acid (1.0 g) as a yellow oil. ¹H NMR (400 MHz, DMSO-d₆): δ 8.04 (d, 1H), 7.80 (d, 1H), 5.34 (s, 2H), 3.43 (s, 3H).

### Intermediate 5

### Methyl 2-(4-fluoro-3-(methoxymethoxy)-5-(trifluoromethyl)phenyl)oxazolo[5,4-c]pyridine-6-carboxylate

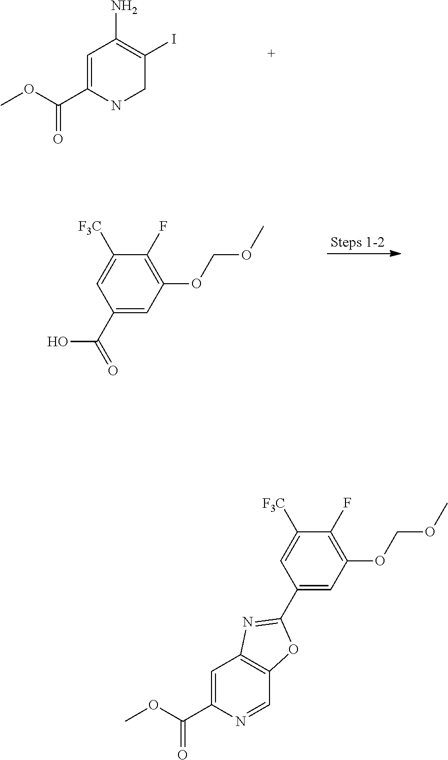

### Step 1: Methyl 4-(4-fluoro-3-(methoxymethoxy)-5-(trifluoromethyl)benzamido)-5-iodopicolinate

T₃P (50% in EtOAc, 6.15 mmol) and TEA (3.77 g, 37.2 mmol) were added to a mixture of Intermediate 4 (500 mg, 1.86 mmol) and methyl 4-amino-5-iodopicolinate (518 mg, 1.86 mmol) in DCM (5 mL). The mixture was stirred at rt overnight, slowly poured into H₂O (50 mL), and then extracted (3×40 mL EtOAc). The combined organic layers were washed (100 mL brine), dried (Na₂SO₄), filtered, and then concentrated. The residue was purified by silica gel chromatography (60% EtOAc/petroleum ether) to give methyl 4-(4-fluoro-3-(methoxymethoxy)-5-(trifluoromethyl)benzamido)-5-iodopicolinate (240 mg, 21%) as a white solid. ¹H NMR (400 MHz, DMSO-d₆): δ 10.50 (s, 1H), 9.09 (s, 1H), 8.26 (s, 1H), 8.16 (d, 1H), 8.04 (d, 1H), 5.44 (s, 2H), 3.89 (s, 3H), 3.46 (s, 3H).

### Step 2: Methyl 2-(4-fluoro-3-(methoxymethoxy)-5-(trifluoromethyl)phenyl)oxazolo[5,4-c]pyridine-6-carboxylate

CuI (7.9 mg, 0.041 mmol), 1,10-phenanthroline (3.7 mg, 0.02 mmol), and Cs₂CO₃ (136 mg, 0.42 mmol) were added to a mixture of methyl 4-(4-fluoro-3-(methoxymethoxy)-5-(trifluoromethyl)benzamido)-5-iodopicolinate (110 mg, 0.208 mmol) in DME (5 mL) under N₂. The mixture was heated at 90° C. overnight and filtered. The filtrate was concentrated and then purified by silica gel chromatography (60% EtOAc/petroleum ether) to give methyl 2-(4-fluoro-3-(methoxymethoxy)-5-(trifluoromethyl)phenyl)oxazolo[5,4-c]pyridine-6-carboxylate (27 mg, 32%) as a yellow oil. LCMS: 401.1 [M+H]⁺.

The Intermediates below were synthesized from Intermediate 4 in a similar manner to that described for Intermediate 5.

| Int | Structure | Name | [M + H]⁺ |
|---|---|---|---|
| 5.01 | 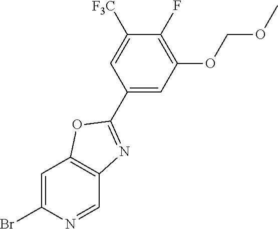 [SMILES: COCOc1cc(-c2nc3cnc(Br)cc3o2)cc(C(F)(F)F)c1F] | 6-Bromo-2-(4-fluoro-3- (methoxymethoxy)-5- (trifluoromethyl)phenyl)oxazolo[4,5- c]pyridine | 420.9 |
| 5.02 (1,2) | 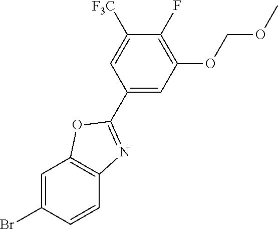 [SMILES: COCOc1cc(-c2nc3ccc(Br)cc3o2)cc(C(F)(F)F)c1F] | 6-Bromo-2-(4-fluoro-3- (methoxymethoxy)-5- (trifluoromethyl)phenyl)benzo[d]oxa- zole | 420.0 |

Alternate conditions used:
1. Step 1: HATU, DIEA, DMF, rt, 1 h;
2. Step 2: DIEA, PPh₃, THF, rt, ON.

### Intermediate 6

### 2-(3-(Benzyloxy)-4,5-difluorophenyl)benzo[d]oxazole-5-carboxylic Acid

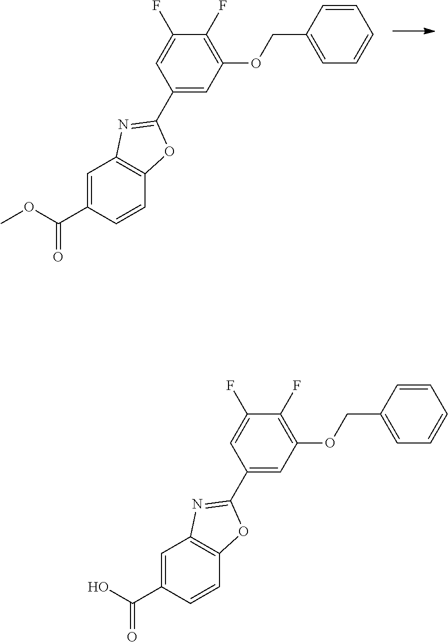 [SMILES: COC(=O)c1ccc2oc(-c3cc(F)c(F)c(OCc4ccccc4)c3)nc2c1 ; O=C(O)c1ccc2oc(-c3cc(F)c(F)c(OCc4ccccc4)c3)nc2c1]

Sodium hydroxide (2 N, 5.0 mL, 10.0 mmol) was added to a suspension of Intermediate 1 (800 mg, 2.02 mmol) in THF (20 mL) and methanol (5 mL) at rt. The mixture was stirred for 3 days, concentrated to remove organics, diluted (20 mL water), and then acidified (˜1 mL conc. HCl). The precipitate was collected by filtration and air dried to give 2-(3-(benzyloxy)-4,5-difluorophenyl)benzo[d] oxazole-5-carboxylic acid (760 mg, 98%) as a gray solid. ¹H NMR (400 MHz, DMSO-d₆) δ:13.53-12.93 (m, 1H), 8.33 (d, J=1.3 Hz, 1H), 8.11-8.06 (m, 1H), 7.96-7.84 (m, 3H), 7.56-7.52 (m, 2H), 7.49-7.43 (m, 2H), 7.42-7.38 (m, 1H), 5.45-5.39 (m, 2H); LCMS 381.9 [M+H]⁺.

The Intermediates below were synthesized in a similar manner to that described for Intermediate 6.

| Int | Structure | Name | [M + H]⁺ |
|---|---|---|---|
| 6.01 | 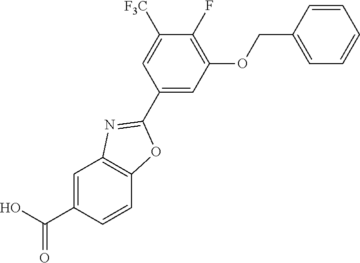 [SMILES: O=C(O)c1ccc2oc(-c3cc(OCc4ccccc4)c(F)c(C(F)(F)F)c3)nc2c1] | 2-(3-(Benzyloxy)-4-fluoro-5- (trifluoromethyl)phenyl)benzo[d]oxa- zole-5-carboxylic acid | 432.0 |
| 6.02 | 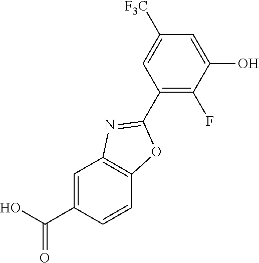 [SMILES: O=C(O)c1ccc2oc(-c3cc(C(F)(F)F)cc(O)c3F)nc2c1] | 2-(2-Fluoro-3-hydroxy-5- (trifluoromethyl)phenyl)benzo[d]oxa- zole-5-carboxylic acid | 341.9 |
| 6.03 | 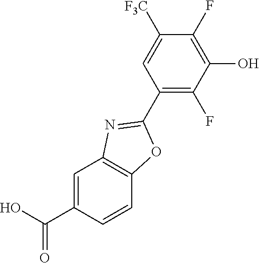 [SMILES: O=C(O)c1ccc2oc(-c3cc(C(F)(F)F)c(F)c(O)c3F)nc2c1] | 2-(2,4-Difluoro-3-hydroxy-5- (trifluoromethyl)phenyl)benzo[d]oxa- zole-5-carboxylic acid | 359.8 |
| 6.04 (1) | 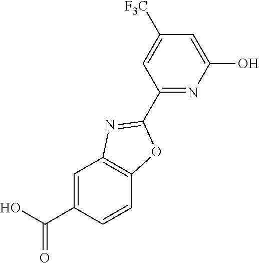 [SMILES: O=C(O)c1ccc2oc(-c3cc(C(F)(F)F)cc(O)n3)nc2c1] | 2-(6-Hydroxy-4- (trifluoromethyl)pyridin-2- yl)benzo[d]oxazole-5-carboxylic acid | 324.9 |

Alternate conditions used:
1. 1 N NaOH, THF, rt, ON.

### Intermediate 7

### 5-(4-(Methylsulfonyl)piperazin-1-yl)benzo[d]oxazole

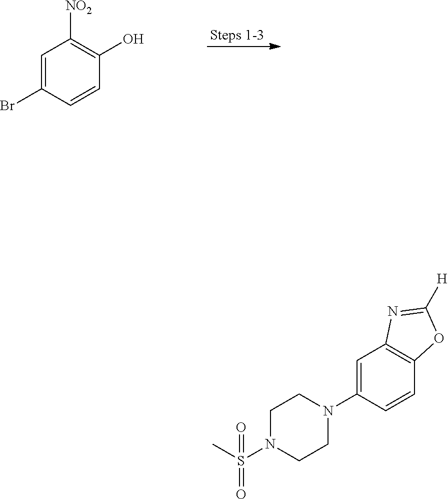 [SMILES: O=[N+]([O-])c1cc(Br)ccc1O ; CS(=O)(=O)N1CCN(c2ccc3ocnc3c2)CC1]

### Step 1: 4-(4-(Methylsulfonyl)piperazin-1-yl)-2-nitrophenol

A mixture of 4-bromo-2-nitrophenol (1.00 g, 4.59 mmol), 1-methanesulfonylpiperazine (1.12 g, 6.82 mmol), RuPhos (0.22 g, 0.46 mmol), NaO^{t}Bu (1.33 g, 13.8 mmol), Pd₂(dba)₃ (0.21 g, 0.23 mmol), and dioxane (10 mL) was degassed by bubbling nitrogen through the suspension for 5 min, heated at 90° C. for 3 h, cooled to rt, diluted (100 mL EtOAc), washed (100 mL saturated NH₄Cl and then 100 mL brine), dried (Na₂SO₄), and then concentrated. The residue was purified by silica gel chromatography (10-50% EtOAc/hexanes) to give 4-(4-(methylsulfonyl)piperazin-1-yl)-2-nitrophenol (1.38 g, 69%) as a reddish-brown solid. ¹H NMR (400 MHz, DMSO-d₆) δ 10.39 (s, 1H), 7.40-7.32 (m, 2H), 7.06 (d, J=8.9 Hz, 1H), 3.24 (br d, J=4.2 Hz, 4H), 3.22-3.14 (m, 4H), 2.93 (s, 3H); LCMS 301.9 [M+H]⁺.

### Step 2: 2-Amino-4-(4-(methylsulfonyl)piperazin-1-yl)phenol

A mixture of 4-(4-(methylsulfonyl)piperazin-1-yl)-2-nitrophenol (950 mg, 3.15 mmol), palladium on carbon (10%, 95 mg), THF (30 mL), and ethanol (30 mL) was stirred under a balloon of hydrogen for 90 min then filtered through a plug of Celite. The filter cake was rinsed (30 mL THF), and the filtrate was concentrated to give 2-amino-4-(4-(methylsulfonyl)piperazin-1-yl)phenol (855 mg, 82%) as a brown solid. ¹H NMR (400 MHz, DMSO-d₆): δ 8.49 (s, 1H), 6.52 (d, J=8.3 Hz, 1H), 6.29 (s, 1H), 6.03 (m, J=8.6 Hz, 1H), 4.45 (br s, 2H), 3.25-3.17 (m, 4H), 3.02-2.96 (m, 4H), 2.96-2.86 (m, 3H); LCMS 271.9 [M+H]⁺.

### Step 3: 5-(4-(Methylsulfonyl)piperazin-1-yl)benzo[d]oxazole

A mixture of 2-amino-4-(4-(methylsulfonyl)piperazin-1-yl)phenol (200 mg, 0.74 mmol) and triethylorthoformate (1 mL) was heated at 110° C. in a microwave for 30 min, concentrated, and then purified by silica gel chromatography (10-80% EtOAc/hexanes) to give 5-(4-(methylsulfonyl)piperazin-1-yl)benzo[d]oxazole (123 mg, 59%) as a beige solid. ¹H NMR (400 MHz, DMSO-d₆): δ 8.65 (s, 1H), 7.64 (d, J=8.9 Hz, 1H), 7.34 (s, 1H), 7.19-7.14 (m, 1H), 3.27 (m, 8H), 2.94 (s, 3H); LCMS 281.8 [M+H]⁺.

### Intermediate 8

### 5-Bromo-2-(3-methoxy-5-(trifluoromethyl)phenyl)benzo[d]oxazole

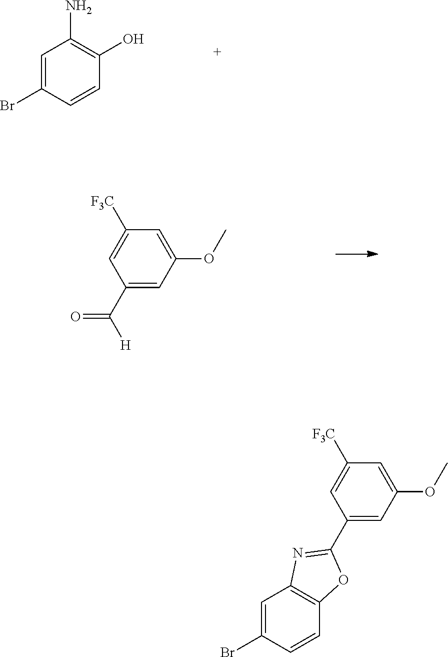 [SMILES: Nc1cc(Br)ccc1O ; COc1cc(C=O)cc(C(F)(F)F)c1 ; COc1cc(-c2nc3cc(Br)ccc3o2)cc(C(F)(F)F)c1]

Copper(II) acetate (44 mg, 0.24 mmol) was added to a solution of 3-methoxy-5-(trifluoromethyl)benzaldehyde (500 mg, 2.45 mmol) and 2-amino-4-bromophenol (507 mg, 2.69 mmol) in toluene (10 mL). The mixture was heated at 110° C. for 3 h, cooled to rt, poured into water (30 mL), and then extracted (3×30 mL EtOAc). The combined organic layers were washed (2×30 mL brine), dried (Na₂SO₄), filtered, and concentrated. The residue was purified by silica gel chromatography (petroleum ether/ethyl acetate=90/1 to 70/1) to give 5-bromo-2-(3-methoxy-5-(trifluoromethyl)phenyl)benzo[d]oxazole (505 mg, 55%) as a yellow solid. ¹H NMR (400 MHz, DMSO-d₆): δ 8.11 (d, 1H), 8.00 (s, 1H), 7.99 (s, 1H), 7.83 (d, 1H), 7.64-7.63 (m, 1H), 7.56 (s, 1H), 3.97 (s, 3H); LCMS: 371.9 [M+H]⁺.

The Intermediates below were synthesized in a similar manner to that described for Intermediate 8.

| Int | Structure | Name | [M + H]⁺ |
|---|---|---|---|
| 8.01 | 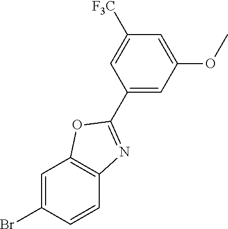 [SMILES: COc1cc(-c2nc3ccc(Br)cc3o2)cc(C(F)(F)F)c1] | 6-Bromo-2-(3-methoxy-5- (trifluoromethyl)phenyl)benzo[d]oxa- zole | 372.0 |
| 8.02 | 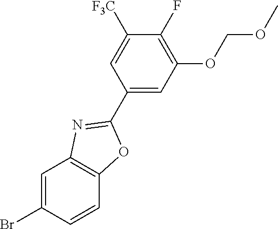 [SMILES: COCOc1cc(-c2nc3cc(Br)ccc3o2)cc(C(F)(F)F)c1F] | 5-Bromo-2-(4-fluoro-3- (methoxymethoxy)-5- (trifluoromethyl)phenyl)benzo[d]oxa- zole | 419.9 |

### Intermediate 9

### 4-Fluoro-3-(methoxymethoxy-5-(trifluoromethyl)benzaldehyde

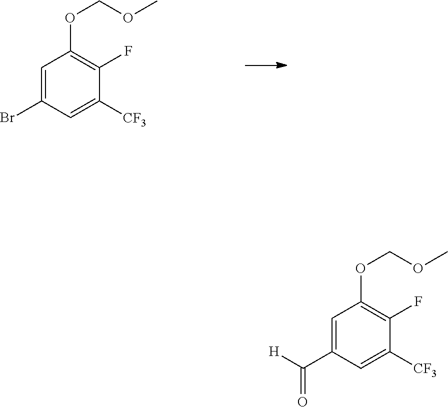 [SMILES: COCOc1cc(Br)cc(C(F)(F)F)c1F ; COCOc1cc(C=O)cc(C(F)(F)F)c1F]

n-Butyllithium solution (2.6 mL, 6.5 mmol, 2.5 Min n-hexane) was added to a mixture of 5-bromo-2-fluoro-1-(methoxymethoxy)-3-(trifluoromethyl)benzene (2.0 g, 6.60 mmol) and THE (15 mL) at −78° C. The mixture was degassed with 3 vacuum/N₂ cycles and stirred at −78° C. for 0.5 h. After the addition of DMF (1.0 mL, 13.2 mmol), the mixture was stirred at −78° C. for 0.5 h, poured into saturated NH₄Cl (40 mL), and then extracted (3×60 mL EtOAc). The combined organic layers were washed (80 mL brine), dried (Na₂SO₄), filtered, and then concentrated. The residue was purified by silica gel chromatography (5% EtOAc/petroleum ether) to give 4-fluoro-3-(methoxymethoxy)-5-(trifluoromethyl)benzaldehyde (233 mg, 14%) as a yellow oil. ¹H NMR (400 MHz, DMSO-d₆): 10.01 (s, 1H), 8.05 (d, 1H), 7.99 (d, 1H), 5.44 (s, 2H), 3.45 (s, 3H).

### Intermediate 10

### 3-(Benzyloxy)-5-(trifluoromethyl)benzoic Acid

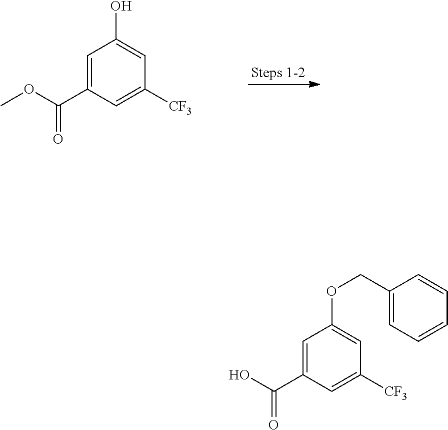 [SMILES: COC(=O)c1cc(O)cc(C(F)(F)F)c1 ; O=C(O)c1cc(OCc2ccccc2)cc(C(F)(F)F)c1]

### Step 1: Methyl 3-(benzyloxy)-5-(trifluoromethyl)benzoate

Benzyl bromide (0.90 mL, 7.79 mmol) was added to a mixture of methyl 5-hydroxy-3-(trifluoromethyl)benzoate (1.14 g, 5.18 mmol), K₂CO₃ (1.48 g, 10.7 mmol), and acetone (50 mL) at rt. The mixture was stirred overnight and then concentrated. The residue was dissolved (100 mL EtOAc), washed (75 mL water and then 75 mL brine), dried (Na₂SO₄), concentrated, and then purified by silica gel chromatography (0-5% EtOAc/hexanes) to give methyl 3-(benzyloxy)-5-(trifluoromethyl)benzoate (1.47 g, 92%) as a colorless oil. ¹H NMR (400 MHz, DMSO-d₆): δ 7.81 (s, 1H), 7.79-7.77 (m, 1H), 7.67 (s, 1H), 7.53-7.47 (m, 2H), 7.46-7.39 (m, 2H), 7.39-7.33 (m, 1H), 5.29 (s, 2H), 3.90 (s, 3H).

### Step 2: 3-(Benzyloxy)-5-(trifluoromethyl)benzoic Acid

A mixture of methyl 3-(benzyloxy)-5-(trifluoromethyl)benzoate (1.47 g, 4.74 mmol) and NaOH (2 M, 12 mL, 24 mmol) in THF (25 mL) and methanol (12 mL) was stirred at rt overnight, concentrated, diluted (50 mL water), and then acidified (1 N HCl, 25 mL). The precipitate was collected by filtration to give 3-(benzyloxy)-5-(trifluoromethyl)benzoic acid (1.31 g, 93%) as a white solid. ¹H NMR (400 MHz, DMSO-d₆): δ 13.54 (br s, 1H), 7.78 (d, J=8.2 Hz, 2H), 7.63 (s, 1H), 7.54-7.46 (m, 2H), 7.46-7.39 (m, 2H), 7.39-7.32 (m, 1H), 5.28 (s, 2H).

### Intermediate 11

### 2-(2,4-Difluoro-3-(methoxymethoxy)-5-(trifluoromethyl)phenyl)-4,4,5,5-tetramethyl-1,3,2-dioxaborolane

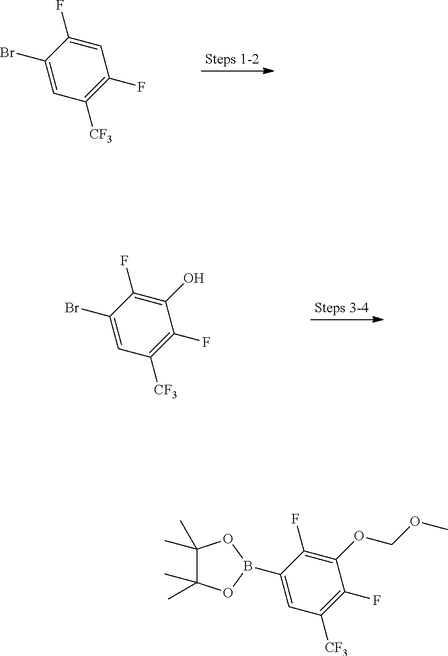

### Step 1: 2-(3-Bromo-2,6-difluoro-5-(trifluoromethyl)phenyl)-4,4,5,5-tetramethyl-1,3,2-dioxaborolane

A mixture of (1,5-cyclooctadiene)(methoxy)iridium(I) dimer (1.13 g, 1.71 mmol), 4,4′-di-tert-butyl-2,2′-bipyridine (0.46 g, 1.71 mmol), and bis(pinacolato)diboron (23.9 g, 94 mmol) was evacuated and refilled with nitrogen 3 times. Cyclopentyl methyl ether (90 mL) was added, and the mixture was evacuated and purged with nitrogen an additional 3 times. 4-Bromo-1,5-difluoro-2-(trifluoromethyl)benzene (22.3 g, 85 mmol) was added. The reaction was heated at 100° C. overnight, cooled to rt, concentrated under reduced pressure, and then purified by silica gel chromatography (0-20% EtOAc/heptane) to give 2-(3-bromo-2,6-difluoro-5-(trifluoromethyl)phenyl)-4,4,5,5-tetramethyl-1,3,2-dioxaborolane (29.3 g, 84%) as a white solid. ¹H NMR (400 MHz, DMSO-d₆): δ 8.32 (t, J=7.4 Hz, 1H), 1.32 (s, 12H).

### Step 2: 3-Bromo-2,6-difluoro-5-(trifluoromethyl)phenol

Hydrogen peroxide (30 w/w in H₂O, 69 mL) was slowly added to a solution of 2-(3-bromo-2,6-difluoro-5-(trifluoromethyl)phenyl)-4,4,5,5-tetramethyl-1,3,2-dioxaborolane (23.6 g, 61 mmol) in methanol (240 mL). The clear solution was stirred at rt for 5 h, quenched by the slow addition of saturated aqueous Na₂S₂O₃ solution over ˜1 h, stirred for 30 min, and then extracted twice with EtOAc. The combined organic layers were washed with brine, dried (MgSO₄), filtered, concentrated under reduced pressure, and then purified by silica gel chromatography (0-20% EtOAc/heptane) to give 3-bromo-2,6-difluoro-5-(trifluoromethyl)phenol (16.9 g, 73%) as a semi-solid. ¹H NMR (400 MHz, DMSO-d₆): δ 11.62 (s, 1H), 7.56 (t, J=6.8 Hz, 1H).

### Step 3: 1-Bromo-2,4-difluoro-3-(methoxymethoxy)-5-(trifluoromethyl)benzene

Chloromethyl methyl ether (0.51 mL, 6.77 mmol) and DIEA (1.57 mL, 9.0 mmol) were added to a solution of 3-bromo-2,6-difluoro-5-(trifluoromethyl)phenol (1.25 g, 4.51 mmol) in CH₂Cl₂ (10 mL) at 0° C. The reaction was stirred at rt overnight, diluted with water, and then extracted with CH₂Cl₂. The aqueous layer was extracted with CH₂Cl₂. The combined organics were dried (MgSO₄), filtered, concentrated under reduced pressure, and then purified by silica gel chromatography (0-20% EtOAc/heptane) to give 1-bromo-2,4-difluoro-3-(methoxymethoxy)-5-(trifluoromethyl)benzene (0.89 g, 58%) as a colorless oil. ¹H NMR (400 MHz, DMSO-d₆): δ 7.99 (t, J=7.0 Hz, 1H), 5.26 (s, 2H), 3.51 (s, 3H).

### Step 4: 2-(2,4-Difluoro-3-(methoxymethoxy)-5-(trifluoromethyl)phenyl)-4,4,5,5-tetramethyl-1,3,2-dioxaborolane

A mixture of 1-bromo-2,4-difluoro-3-(methoxymethoxy)-5-(trifluoromethyl)benzene (0.47 g, 1.46 mmol), bis(pinacolato)diboron (558 mg, 2.2 mmol), and KOAc (287 mg, 2.92 mmol) in dioxane (5 mL) was evacuated and filled with nitrogen 3 times. Pd(dppf)Cl₂ (54 mg, 0.07 mmol) was added. The mixture was degassed and filled with nitrogen 3 more times, heated at 80° C. for 3 days, cooled to rt, concentrated under reduced pressure, and then purified by silica gel chromatography (0-20% EtOAc/heptanes) to give 2-(2,4-difluoro-3-(methoxymethoxy)-5-(trifluoromethyl)phenyl)-4,4,5,5-tetramethyl-1,3,2-dioxaborolane (0.36 g, 63%) as a colorless oil. ¹H NMR (400 MHz, DMSO-d₆): δ 7.62 (br t, J=6.7 Hz, 1H), 5.21 (s, 2H), 3.50 (s, 3H), 1.44-1.25 (m, 12H).

The Intermediate below was synthesized in a similar manner to that described for Intermediate 11, Step 4.

| Int | Structure | Name | H′NMR |
|---|---|---|---|
| 11.01 | 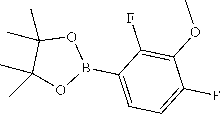 [SMILES: COc1c(F)ccc(B2OC(C)(C)C(C)(C)O2)c1F] | 2-(2,4-Difluoro-3-methoxyphenyl)- 4,4,5,5-tetramethyl-1,3,2- dioxaborolane | ¹H NMR (400 MHz, DMSO-d₆): 7.35-7.20 (m, 1H), 7.15-7.05 (m, 1H), 3.90 (s, 3H), 1.29 (s, 12H) |

Alternate conditions used: 105° C., overnight.

### Intermediate 12

### 2-(3-(Benzyloxy)-2,4-difluoro-5-(trifluoromethyl)phenyl)-4,4,5,5-tetramethyl-1,3,2-dioxaborolane

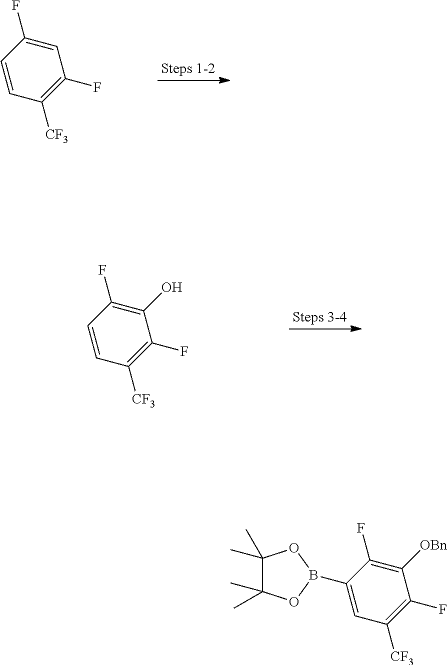 [SMILES: Fc1ccc(C(F)(F)F)c(F)c1 ; Oc1c(F)ccc(C(F)(F)F)c1F ; CC1(C)OB(c2cc(C(F)(F)F)c(F)c(OCc3ccccc3)c2F)OC1(C)C]

### Step 1: (2,6-Difluoro-3-(trifluoromethyl)phenyl)boronic Acid

n-Butyllithium (2.5 M in hexanes, 171 mL, 428 mmol) was added dropwise to a mixture of 2,4-difluoro-1-(trifluoromethyl)benzene (60.0 g, 330 mmol) in Et₂O (˜400 mL) at −78° C. under N₂. The reaction was stirred for 1 h. Trimethyl borate (44.7 mL, 395 mmol) in Et₂O (200 mL) was added dropwise at −78° C. The reaction was stirred at for 1 h, allowed to warm to rt slowly, stirred for 10 h, and then quenched slowly with aq. HCl (1 M, 500 mL) under ice cooling. The organic layer was separated and washed with brine (300 mL) to give (2,6-difluoro-3-(trifluoromethyl)phenyl)boronic acid as a solution in Et₂O (˜600 mL). LCMS: 225.1 [M−H]⁻.

### Step 2: 2,6-Difluoro-3-(trifluoromethyl)phenol

Hydrogen peroxide (166 mL, 1.72 mol, 30% purity in H₂O) was added to a solution of (2,6-difluoro-3-(trifluoromethyl)phenyl)boronic acid (74.4 g, 329 mmol) in Et₂O (˜600 mL) at 0° C. The mixture was heated to 40° C., stirred for 4 h, and then allowed to cool to rt. The aqueous layer was separated. The organic layer was cooled to 0° C. and then quenched with aqueous Na₂SO₃ (20% in H₂O, −500 mL) keeping the temperature <20° C. The organic layer was separated. The aqueous layer was extracted with EtOAc (2×300 ml). The organic layers were combined, washed with water (2×300 ml), washed with brine (300 ml), dried (Na₂SO₄), filtered, concentrated, and then purified by silica gel chromatography (petroleum ether/ethyl acetate=50:1 to 5:1) to give 2,6-difluoro-3-(trifluoromethyl)phenol (41.3 g, 63%) as a yellow oil. ¹H NMR (400 MHz, DMSO-d₆): δ 11.01 (s, 1H), 7.27-7.19 (m, 2H); LCMS: 196.9 [M−H]⁻.

### Step 3: 2-(Benzyloxy)-1,3-difluoro-4-(trifluoromethyl)benzene

Benzyl bromide (43.2 mL, 363 mmol) was added to a mixture of 2,6-difluoro-3-(trifluoromethyl)phenol (60.3 g, 303 mmol), K₂CO₃ (126 g, 909 mmol), and DMF (600 mL) at rt. The mixture was stirred at 50° C. for 12 h, cooled to rt, poured into H₂O (500 mL), and then extracted with EtOAc (3×300 mL). The organic layers were combined, washed with brine (300 mL), dried (Na₂SO₄), filtered, concentrated, and then purified by silica gel chromatography (petroleum ether/ethyl acetate=100:1 to 10:1) to give 2-(benzyloxy)-1,3-difluoro-4-(trifluoromethyl)benzene (54.5 g, 62%) as a yellow oil. ¹H NMR (400 MHz, DMSO-d₆): δ 7.56-7.50 (m, 1H), 7.43-7.34 (m, 6H), 5.24 (s, 2H).

### Step 4: 2-(3-(Benzyloxy)-2,4-difluoro-5-(trifluoromethyl)phenyl)-4,4,5,5-tetramethyl-1,3,2-dioxaborolane

(1,5-Cyclooctadiene)(methoxy)iridium(I) dimer (92.1 mg, 0.139 mmol) and 4,4′-di-tert-butyl-2,2′-bipyridine (37.2 mg, 0.139 mmol) were added to a solution of 2-(benzyloxy)-1,3-difluoro-4-(trifluoromethyl)benzene (4.01 g, 13.88 mmol) and 4,4,4′,4′,5,5,5′,5′-octamethyl-2,2′-bi(1,3,2-dioxaborolane) (2.93 g, 11.52 mmol) in THF (40 mL) under N₂. The mixture was stirred at 80° C. for 4 h, cooled to rt, concentrated, and then purified by silica gel chromatography (petroleum ether/ethyl acetate=100/1 to 10/1) to give 2-(3-(benzyloxy)-2,4-difluoro-5-(trifluoromethyl)phenyl)-4,4,5,5-tetramethyl-1,3,2-dioxaborolane (5.3 g, 92%) as a white solid. ¹H NMR (400 MHz, DMSO-d₆): δ 7.45 (t, 1H), 7.43-7.38 (m, 5H), 5.22 (s, 2H), 1.31 (s, 12H).

The Intermediate below was synthesized from 2,4-difluoro-1-(trifluoromethyl)benzene following the procedures described for Intermediate 12 (Steps 1-2), Intermediate 11 (Step 3), and then Intermediate 12 (Step 4).

| Int | Structure | Name |
|---|---|---|
| 11 | 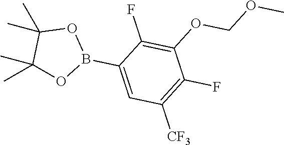 | 2-(2,4-Difluoro-3- (methoxymethoxy)-5- (trifluoromethyl)phenyl)-4,4,5,5- tetramethyl-1,3,2-dioxaborolane |

### Intermediate 13

### 4-(Benzyloxy)-3,5-difluoro-2-(tributylstannyl)-6-(trifluoromethyl)pyridine

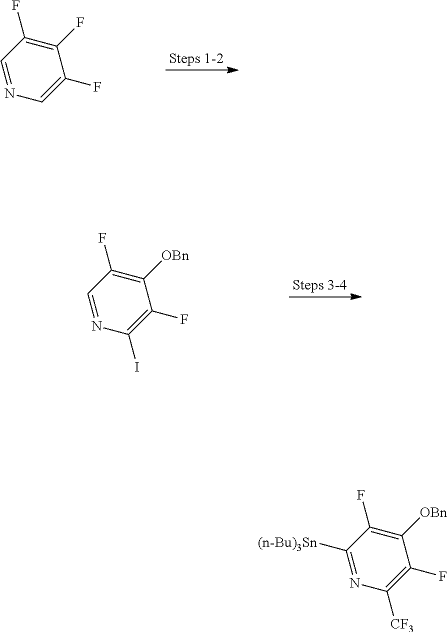

### Step 1: 4-(Benzyloxy)-3,5-difluoropyridine

Sodium hydride (1.32 g, 33.07 mmol, 60% purity) was added in portions to a mixture of 3,4,5-trifluoropyridine (4.01 g, 30.06 mmol) and BnOH (3.58 g, 33.07 mmol) in DMF (50 mL) at rt under N₂. The mixture was stirred at rt for 1 h, poured into H₂O (40 mL) slowly, and then extracted with ethyl acetate (4×20 mL). The organic layers were washed with brine (20 mL), dried over Na₂SO₄, filtered, concentrated, and then purified by silica gel chromatography (petroleum ether/ethyl acetate=20:1 to 13:1) to give 4-(benzyloxy)-3,5-difluoropyridine (6.20 g, 93%) as colorless liquid. ¹H NMR (400 MHz, CDCl₃): δ 8.25 (s, 2H), 7.47-7.34 (m, 5H), 5.42 (s, 2H); LCMS: 222.1 [M+H]⁺.

### Step 2: 4-(Benzyloxy)-3,5-difluoro-2-iodopyridine

n-Butyllithium (7.05 mL, 17.63 mmol, 2.5 M in hexanes) was added dropwise to a mixture of 4-(benzyloxy)-3,5-difluoropyridine (3.02 g, 13.56 mmol) in THF (35 mL) at −78° C. under N₂. The reaction was stirred for 1 h. Iodine (5.16 g, 20.34 mmol) in THF (10 mL) was added dropwise at −78° C. The resulting mixture was stirred for 1 h, allowed to warm to rt, added into sat.aq Na₂SO₃ (80 mL) slowly, and then extracted with ethyl acetate (4×20 mL). The combined organic layers were washed with brine (80 mL), dried over Na₂SO₄, filtered, concentrated, and then purified by column chromatography (SiO₂, petroleum ether/ethyl acetate=50/1 to 20/1) to give 4-(benzyloxy)-3,5-difluoro-2-iodopyridine (1.80 g, 38%) as a yellow solid. ¹H NMR (400 MHz, CDCl₃): δ 8.10 (s, 1H), 7.46-7.35 (m, 5H), 5.41 (s, 2H); LCMS: 347.9 [M+H]⁺.

### Step 3: 4-(Benzyloxy)-3,5-difluoro-2-(trifluoromethyl)pyridine

Methyl 2,2-difluoro-2-(fluorosulfonyl)acetate (5.45 g, 28.4 mmol) and CuI (5.40 g, 28.4 mmol) were added to a solution of 4-(benzyloxy)-3,5-difluoro-2-iodopyridine (1.97 g, 5.68 mmol) in DMF (20 mL) under N₂. The mixture was stirred at 70° C. for 4 h and then allowed to cool to rt. The solids were removed by filtration. The filtrate was diluted with ethyl acetate (20 mL) and aqueous NH₃·H₂O (100 mL, 9% aq. solution). The aqueous layer was separated and extracted with diethyl ether (10 mL). The combined organic layers were washed with aqueous NH₃·H₂O (3×20 mL, 9% aq. solution), washed with water (50 mL), washed with brine (50 mL), dried (Na₂SO₄), filtered, concentrated, and then purified by silica gel chromatography (petroleum ether/ethyl acetate=I/O to 10/1) to give 4-(benzyloxy)-3,5-difluoro-2-(trifluoromethyl)pyridine (1.30 g, 79%) as a colorless liquid. ¹H NMR (400 MHz, CDCl₃): δ 8.30 (s, 1H), 7.49-7.34 (m, 5H), 5.48 (s, 2H); LCMS: 290.0 [M+H]⁺.

### Step 4: 4-(Benzyloxy)-3,5-difluoro-2-(tributylstannyl)-6-(trifluoromethyl)pyridine

Lithium diisopropylamide (3.4 mL, 6.74 mmol, 2 M in THF) was added dropwise to a solution of 4-(benzyloxy)-3,5-difluoro-2-(trifluoromethyl)pyridine (1.30 g, 4.50 mmol) in THF (15 mL) at −78° C. under N₂. The mixture was stirred at −78° C. for 0.5 h. n-Bu₃SnCl (4.8 mL, 17.98 mmol) was added dropwise. The mixture was stirred for 1 h, quenched with sat. KF (50 mL), and then stirred at rt for 0.5 h. The solids were filtered, and the filter cake was washed with ethyl acetate (20 mL). The filtrate was extracted with ethyl acetate (2×20 mL). The combined organic layers were washed with brine (50 mL), dried over Na₂SO₄, filtered, concentrated, and then purified by silica gel chromatography (petroleum ether) to give 4-(benzyloxy)-3,5-difluoro-2-(tributylstannyl)-6-(trifluoromethyl)pyridine (1.60 g, 61%) as a colorless liquid. ¹H NMR (400 MHz, CDCl₃): δ 7.48-7.31 (m, 5H), 5.40 (s, 2H), 1.62-1.49 (m, 6H), 1.38-1.28 (m, 6H), 1.22-1.09 (m, 6H), 0.89 (t, 9H); LCMS: 580.2 [M+H]⁺.

### Intermediate 14

### 6-Bromo-3-iodo-1-(tetrahydro-2H-pyran-2-yl)-1H-indazole

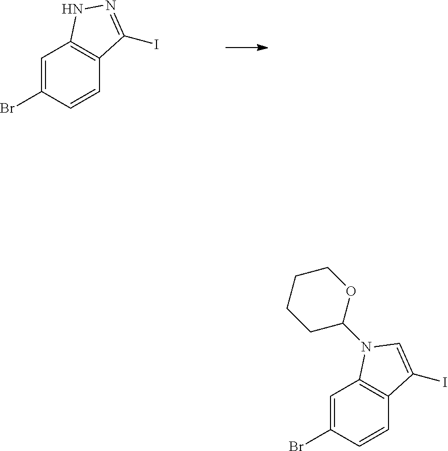 [SMILES: Brc1ccc2c(I)n[nH]c2c1 ; Brc1ccc2c(I)cn(C3CCCCO3)c2c1]

p-Toluenesulfonic acid (12 mg, 0.062 mmol) was added to a mixture of 6-bromo-3-iodo-1H-indazole (2.0 g, 6.19 mmol) in THF (18 mL). The mixture was heated at 70° C. for 1 h. 3,4-Dihydro-2H-pyran (521 mg, 6.19 mmol) was added. The mixture was heated at 70° C. overnight, allowed to cool to rt, diluted (5 mL saturated NaHCO₃ and then 80 mL water), and then extracted (4×20 mL EtOAc). The combined organic layers were concentrated and then purified by silica gel chromatography (0-5% EtOAc/petroleum ether) to give 6-bromo-3-iodo-1-(tetrahydro-2H-pyran-2-yl)-1H-indazole (1.59 g, 63%) as a white solid. ¹H NMR (400 MHz, DMSO-d₆): δ 7.79-7.74 (m, 1H), 7.66-7.62 (m, 2H), 5.87 (d, 1H), 3.89-3.81 (m, 1H), 3.78-3.71 (m, 1H), 2.40-2.27 (m, 1H), 2.06-1.92 (m, 2H), 1.80-1.64 (m, 1H), 1.63-1.51 (m, 2H); LCMS: 407.0 [M+H]⁺.

The Intermediate below was synthesized from 6-bromo-3-iodo-1H-pyrazolo[4,3-c]pyridine in a similar manner to that described for Intermediate 14.

| Int | Structure | Name | [M + H]⁺ |
|---|---|---|---|
| 14.01 | 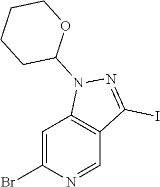 [SMILES: Brc1cc2c(cn1)c(I)nn2C1CCCCO1] | 6-Bromo-3-iodo-1-(tetrahydro-2H- pyran-2-yl)-1H-pyrazolo[4,3- c]pyridine | 407.9 |

### Intermediate 15

### 6-Chloro-5-fluoro-3-iodo-1-methyl-1H-pyrazolo[3,4-b]pyridine

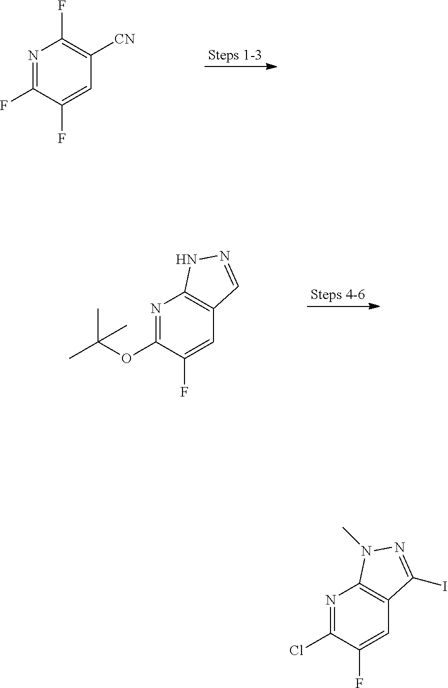

### Step 1: 6-(tert-Butoxy)-2,5-difluoronicotinonitrile

A solution of t-BuOK (20 g, 178 mmol), t-BuOH (140 mL), and THF (30 mL) was added to a solution of 2,5,6-trifluoronicotinonitrile (25 g, 158 mmol), DMSO (7.34 g, 94.0 mmol), t-BuOH (180 mL), and THF (40 mL) at 0° C. The mixture was allowed to warm to rt, stirred for 2 h, poured into water (600 mL), and then extracted with EtOAc (2×400 mL). The organic layers were combined, washed with water (2×100 mL), washed with brine (100 mL), dried (Na₂SO₄), filtered, concentrated, and then purified by silica gel chromatography (petroleum ether/EtOAc=50/1) to give 6-(tert-butoxy)-2,5-difluoronicotinonitrile (22 g, 65%) as a yellow oil. ¹H NMR (400 MHz, DMSO-d₆): δ 8.47 (dd, 1H), 1.61 (s, 9H); LCMS: 213.1 [M+H]⁺.

### Step 2: 6-(tert-Butoxy)-2,5-difluoronicotinaldehyde

DIBAL-H (1 M in toluene, 122 mL, 122 mmol) was added to a solution of 6-(tert-butoxy)-2,5-difluoronicotinonitrile (17 g, 80 mmol) in DCM (350 mL) at −78° C. The mixture was allowed to warm to rt for 5 h, poured into sat. aq. Seignette salt (500 mL), and then extracted with EtOAc (2×300 mL). The organic layers were combined, washed with water (2×100 mL), washed with brine (100 mL), dried (Na₂SO₄), filtered, concentrated, and then purified by silica gel chromatography (petroleum ether/EtOAc=20/1) to give 6-(tert-butoxy)-2,5-difluoronicotinaldehyde (10 g, 58%) as a yellow oil. ¹H NMR (400 MHz, DMSO-d₆): δ 10.00 (s, 1H), 8.18-8.04 (m, 1H), 1.64 (s, 9H).

### Step 3: 6-(tert-Butoxy)-5-fluoro-1H-pyrazolo[3,4-b]pyridine

A mixture of 6-(tert-butoxy)-2,5-difluoronicotinaldehyde (10 g, 46.5 mmol), NH₂NH₂·H₂O (98%, 42 g, 823 mmol), and NMP (120 mL) was stirred at 130° C. for 5 h, allowed to cool to rt, poured into water (700 mL), and then filtered. The filter cake was dissolved in EtOAc (800 mL). The organics were washed with water (2×300 mL), washed with brine (200 mL), dried (Na₂SO₄), filtered, concentrated, and then purified by silica gel chromatography (petroleum ether/EtOAc=10/1) to give 6-(tert-butoxy)-5-fluoro-1H-pyrazolo[3,4-b]pyridine (7.7 g, 79%) as a yellow solid. ¹H NMR (400 MHz, DMSO-d₆): δ 13.36 (br s, 1H), 8.04-7.83 (m, 2H), 1.64 (s, 9H); LCMS: 210.1 [M+H]⁺.

### Step 4: 6-(tert-Butoxy)-5-fluoro-3-iodo-1H-pyrazolo[3,4-b]pyridine

Potassium hydroxide (13.4 g, 239 mmol) was added to a solution of 6-(tert-butoxy)-5-fluoro-1H-pyrazolo[3,4-b]pyridine (10 g, 47.8 mmol), I₂ (24.3 g, 95.6 mmol), and DMF (200 mL) at 0° C. The mixture was allowed to warm to rt overnight, poured into water (500 mL), and then extracted with EtOAc (2×300 mL). The organic layers were combined, washed with water (2×200 mL), washed with brine (200 mL), dried (Na₂SO₄), filtered, concentrated, and then purified by silica gel chromatography (petroleum ether/EtOAc=20/1) to give 6-(tert-butoxy)-5-fluoro-3-iodo-1H-pyrazolo[3,4-b]pyridine (13.23 g, 82%) as a yellow solid. ¹H NMR (400 MHz, DMSO-d₆): δ 13.77 (s, 1H), 7.67 (d, 1H), 1.63 (s, 9H); LCMS: 336.0 [M+H]⁺.

### Step 5: 6-(tert-Butoxy)-5-fluoro-3-iodo-1-methyl-1H-pyrazolo[3,4-b]pyridine

A mixture of 6-(tert-butoxy)-5-fluoro-3-iodo-1H-pyrazolo[3,4-b]pyridine (16.0 g, 47.7 mmol), Mel (13.6 g, 95.5 mmol), K₂CO₃ (26.4 g, 191 mmol), and DMF (160 mL) was stirred at rt overnight, poured into water (600 mL), and then extracted with EtOAc (2×600 mL). The organic layers were combined, washed with water (2×300 mL), washed with brine (300 mL), dried (Na₂SO₄), filtered, concentrated, and then purified by silica gel chromatography (petroleum ether/EtOAc=40/1) to give 6-(tert-butoxy)-5-fluoro-3-iodo-1-methyl-1H-pyrazolo[3,4-b]pyridine (12.7 g, 76%) as a yellow solid and 6-(tert-butoxy)-5-fluoro-3-iodo-2-methyl-2H-pyrazolo[3,4-b]pyridine (1.2 g, 7%) as a yellow solid.

¹H NMR (400 MHz, DMSO-d₆): δ 7.69 (d, 1H), 3.95 (s, 3H), 1.66 (s, 9H); LCMS: 350.0 [M+H]⁺.

¹H NMR (400 MHz, DMSO-d₆): δ 7.58 (d, 1H), 4.07 (s, 3H), 1.59 (s, 9H); LCMS: 350.0 [M+H]⁺.

### Step 6: 6-Chloro-5-fluoro-3-iodo-1-methyl-1H-pyrazolo[3,4-b]pyridine

Phosphorus(V) oxychloride (84.15 g, 548.8 mmol) was added to a mixture of 6-(tert-butoxy)-5-fluoro-3-iodo-1-methyl-1H-pyrazolo[3,4-b]pyridine (8.5 g, 24.4 mmol) in DMF (160 mL) at rt. The mixture was stirred at 100° C. for 3.5 h, allowed to cool to rt, and concentrated. The residual mixture containing some DMF was added dropwise to NaHCO₃ (1000 mL) and then extracted with EtOAc (2×300 mL). The organic layers were combined, washed with water (2×200 mL), washed with brine (200 mL), dried (Na₂SO₄), filtered, concentrated, and then purified by silica gel chromatography (petroleum ether/EtOAc=50/1) to give 6-chloro-5-fluoro-3-iodo-1-methyl-1H-pyrazolo[3,4-b]pyridine (6.66 g, 87%) as a yellow solid. ¹H NMR (400 MHz, DMSO-d₆): δ 8.13 (d, 1H), 4.01 (s, 3H); LCMS: 312.0 [M+H]⁺.

### Intermediate 16

### 6-Chloro-7-fluoro-1H-pyrazolo[4,3-c]pyridine

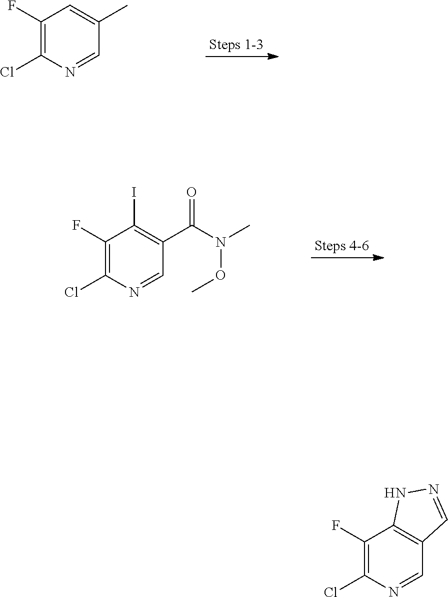

### Step 1: 6-Chloro-5-fluoronicotinic Acid

Potassium permanganate (53.1 g, 335 mmol) was added in one portion to a mixture of 2-chloro-3-fluoro-5-methylpyridine (8.05 g, 54.9 mmol) in pyridine (˜80 mL) and H₂O (˜80 mL) at 20° C. The mixture was heated to 100° C., stirred for 2 h, cooled to 0° C., poured into aq. Na₂S₂O₃ (˜1000 mL), and then stirred for 30 min. The aqueous phase was adjusted to pH-1. The precipitation was filtered, and the filter cake was washed with H₂O (˜30 mL) and then concentrated under vacuum to give 6-chloro-5-fluoronicotinic acid (7.02 g) as a white solid. ¹H NMR (400 MHz, DMSO-d₆) δ 13.91 (s, 1H), 8.76 (d, 1H), 8.30-8.27 (m, 1H); LCMS: 173.9 [M−H]⁻.

### Step 2: 6-Chloro-5-fluoro-4-iodonicotinic Acid

n-Butyllithium (2.5 Min n-hexane, 23.6 mL) was added dropwise over a period of 30 min to a solution of 2,2,6,6-tetramethylpiperidine (10.1 mL, 58.9 mmol) in THF (70 mL) at −78° C. under N₂. The reaction mixture was stirred at −78° C. for 1 h. A mixture of 6-chloro-5-fluoronicotinic acid (6.91 g, 39.3 mmol) in THF (50 mL) was added dropwise over a period of 30 min to the reaction mixture. The reaction mixture was slowly warmed to 20° C., stirred for 3 h, and then cooled to −78° C. A mixture of I₂ (9.98 g, 39.3 mmol) in THE (10 mL) was added dropwise at −78° C. over a period of 30 min to the reaction mixture. The reaction mixture was slowly warmed to 20° C., stirred for additional 10 h, and then poured into sat. aq. NH₄Cl (150 mL). The aqueous phase was extracted with ethyl acetate (3×70 mL). The combined organic phases were washed with brine (200 mL), dried (Na₂SO₄), filtered, and then concentrated. The crude product was triturated with DCM (20 mL) at 20° C. for 30 min. The solid was collected by filtration, and the filter cake was washed with cool DCM (˜5 mL) and then dried to give 6-chloro-5-fluoro-4-iodonicotinic acid (6.03 g, 51%) as a yellow solid. ¹H NMR (400 MHz, DMSO-d₆): δ 7.24 (s, 1H); LCMS: 299.8 [M−H]⁻.

### Step 3: 6-Chloro-5-fluoro-4-iodo-N-methoxy-N-methylnicotinamide

A solution of 6-chloro-5-fluoro-4-iodonicotinic acid (6.02 g, 19.9 mmol, 1.0 eq), N,O-dimethylhydroxylamine hydrochloride (2.33 g, 23.9 mmol), T₃P (26.0 mL, 43.8 mmol, 50% purity in EtOAc), and Et₃N (8.3 mL, 59.7 mmol) in DCM (60 mL) was stirred at 20° C. for 1 h, poured into H₂O (˜100 mL), and then extracted with DCM (3×50 mL). The combined organic phases were washed with brine (150 mL), dried over Na₂SO₄, filtered, concentrated, and then purified by column chromatography (SiO₂, petroleum ether/ethyl acetate=100/1 to 0/1) to give 6-chloro-5-fluoro-4-iodo-N-methoxy-N-methylnicotinamide (3.02 g, 44%) as a yellow solid. ¹H NMR (400 MHz, DMSO-d₆): δ 8.23 (s, 1H), 3.47 (s, 3H), 3.33 (s, 3H); LCMS: 344.9 [M+H]⁺.

### Step 4: 6-Chloro-5-fluoro-4-iodonicotinaldehyde

Diisobutylaluminum hydride solution (1 M in toluene, 8.94 mL, 8.94 mmol) was added to a mixture of 6-chloro-5-fluoro-4-iodo-N-methoxy-N-methylnicotinamide (2.80 g, 8.13 mmol) in DCM (40 mL) at −78° C. under N₂. The mixture was stirred at −78° C. for 2 h, and then poured into 1 M HCl (50 mL). The aqueous phase was extracted with DCM (3×50 mL). The combined organic phases were washed with brine (150 mL), dried with anhydrous Na₂SO₄, filtered, concentrated, and then purified by column chromatography (SiO₂, petroleum ether/ethyl acetate=100/1 to 0/1) to give 6-chloro-5-fluoro-4-iodonicotinaldehyde (1.70 g, 73%) as a yellow solid. ¹H NMR (400 MHz, DMSO-d₆): δ 9.95 (s, 1H), 8.47 (s, 1H); LCMS: 285.9 [M+H]⁺.

### Step 5: (E)-N′-((6-chloro-5-fluoro-4-iodopyridin-3-yl)methylene)-4-methylbenzenesulfonohydrazide

A mixture of 6-chloro-5-fluoro-4-iodonicotinaldehyde (1.20 g, 4.20 mmol) and 4-methylbenzenesulfonohydrazide (861 mg, 4.62 mmol) in EtOH (10 mL) was degassed and purged with N₂ 3 times. The mixture was stirred at 90° C. for 16 h, cooled to 20° C. slowly, diluted with EtOH (5 mL), and then filtered. The filter cake was washed by cool EtOH (˜5 mL) and then dried under reduced pressure to give (E)-N-((6-chloro-5-fluoro-4-iodopyridin-3-yl)methylene)-4-methylbenzenesulfonohydrazide (1.70 g, 89%) as a white solid. ¹H NMR (400 MHz, DMSO-d₆): δ 12.08-11.99 (m, 1H), 8.26 (s, 1H), 8.03 (s, 1H), 7.78 (d, 2H), 7.43 (d, 2H), 2.37 (s, 3H); LCMS: 453.8 [M+H]⁺.

### Step 6: 6-Chloro-7-fluoro-1H-pyrazolo[4,3-c]pyridine

A mixture of (E)-N-((6-chloro-5-fluoro-4-iodopyridin-3-yl)methylene)-4-methylbenzenesulfonohydrazide (1.70 g, 3.75 mmol), Cu₂O (268 mg, 1.87 mmol) in i-AmOH (20 mL) was degassed and purged with N₂ 3 times. The mixture was stirred at 140° C. for 12 h, diluted with H₂O (30 mL), and then extracted with EtOAc (3×20 mL). The combined organic layers were washed with brine (2×30 mL), dried over Na₂SO₄, filtered, concentrated, and then purified by column chromatography (SiO₂, petroleum ether/ethyl acetate=20/1 to 3/1) to give 6-chloro-7-fluoro-1H-pyrazolo[4,3-c]pyridine (250 mg, 39%) as a white solid. ¹H NMR (400 MHz, DMSO-d₆): δ 14.31 (s, 1H), 8.81 (s, 1H), 8.46-8.45 (m, 1H); LCMS: 171.9 [M+H]⁺.

### Intermediate 17

### 6-Bromo-4-fluoro-3-iodo-1-methyl-1H-indazole

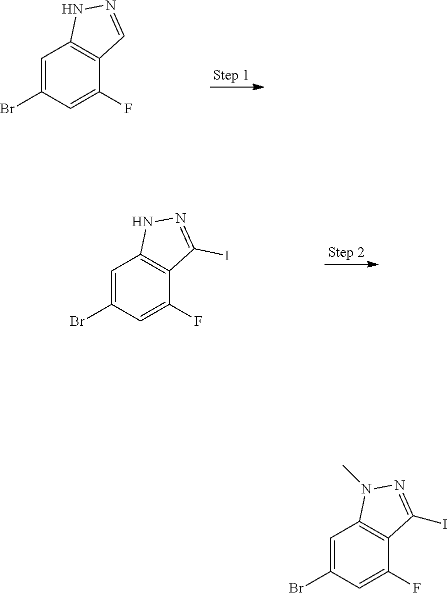

### Step 1: 6-Bromo-4-fluoro-3-iodo-1H-indazole

N-Iodosuccinimide (6.15 g, 27.4 mmol) was added to a solution of 6-bromo-4-fluoro-1H-indazole (4.90 g, 22.8 mmol) in DMF (50 mL) at rt. The mixture was stirred at 80° C. for 2 h, allowed to cool to rt, and then diluted with H₂O (100 mL). The mixture was stirred at rt for 1 h. The solids were filtered, washed with water (300 mL), and then dried under reduced pressure to give 6-bromo-4-fluoro-3-iodo-1H-indazole (7.5 g) as a light red solid. ¹H NMR (400 MHz, DMSO-d₆): δ 13.85 (s, 1H), 7.69 (s, 1H), 7.18 (d, 1H); LCMS: 340.8 [M+H]⁺.

### Step 2: 6-Bromo-4-fluoro-3-iodo-1-methyl-1H-indazole

Potassium carbonate (2.43 g, 17.6 mmol) and Mel (1.67 g, 11.7 mmol) were added to a solution of 6-bromo-4-fluoro-3-iodo-1H-indazole (2.00 g, 5.87 mmol) in DMF (20 mL). The mixture was stirred at rt for 20 h, diluted with H₂O (50 mL), and then extracted with ethyl acetate (2×15 mL). The combined organic layers were washed with brine (30 mL), dried over Na₂SO₄, filtered, concentrated, and then purified by silica gel chromatography (petroleum ether/ethyl acetate=20:1 to 3:1) to give 6-bromo-4-fluoro-3-iodo-1-methyl-1H-indazole (1.23 g, 59%) as a white solid. ¹H NMR (400 MHz, DMSO-d₆): δ 7.94 (s, 1H), 7.21 (dd, 1H), 4.04 (s, 3H); LCMS: 354.8 [M+H]⁺.

The Intermediates below were synthesized in a similar manner to that described for Intermediate 17.

| Int | Structure | Name | [M + H]⁺ |
|---|---|---|---|
| 17.01 | 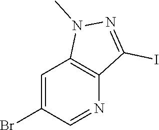 | 6-Bromo-3-iodo-1-methyl-1H- pyrazolo[4,3-b]pyridine | 337.9 |
| 17.02 | 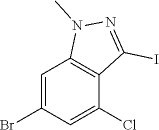 | 6-Bromo-4-chloro-3-iodo-1-methyl- 1H-indazole | 370.9 |
| 17.03 (1) | 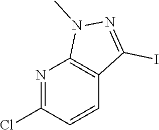 | 6-Chloro-3-iodo-1-methyl-1H- pyrazolo[3,4-b]pyridine | 293.7 |
| 17.04 (2) | 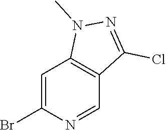 | 6-Bromo-3-chloro-1-methyl-1H- pyrazolo[4,3-c]pyridine | 245.9 |
| 17.05 (3) | 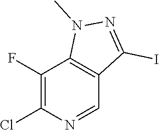 | 6-Chloro-7-fluoro-3-iodo-1-methyl- 1H-pyrazolo[4,3-c]pyridine | 311.8 |
| 17.06 | 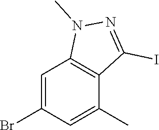 | 6-Bromo-3-iodo-1,4-dimethyl-1H- indazole | 350.9 |
| 17.07 (4) | 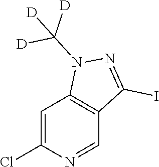 [SMILES: [2H]C([2H])([2H])n1nc(I)c2cnc(Cl)cc21] | 6-Chloro-3-iodo-1-(methyl-d₃)-1H- pyrazolo[4,3-c]pyridine | 297.0 |
| 17.08 (5) | 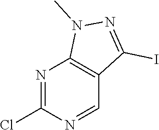 | 6-Chloro-3-iodo-1-methyl-1H- pyrazolo[3,4-d]pyrimidine | 294.7 |
| 17.09 (6) | 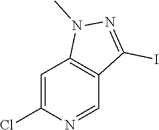 | 6-Chloro-3-iodo-1-methyl-1H- pyrazolo[4,3-c]pyridine | 293.9 |

Alternate conditions used:
1. Step 1: 110° C., 10 h;
2. Step 1: NCS, DMF, rt-80° C., 3 h;
3. Step 1: I₂, K₂CO₃, DMF, rt, ON;
4. Step 2 only: iodomethane-d₃, K₂CO₃, THF, rt, 3.5 h;
5. Step 2 only;
6. Step 2: MeI, Cs₂CO₃, DMF, 50° C., 2 h.

### Intermediate 18

### 6-Chloro-3-(2,4-difluoro-3-(methoxymethoxy)-5-(trifluoromethyl)phenyl)-1-methyl-1H-pyrazolo[4,3-c]pyridine

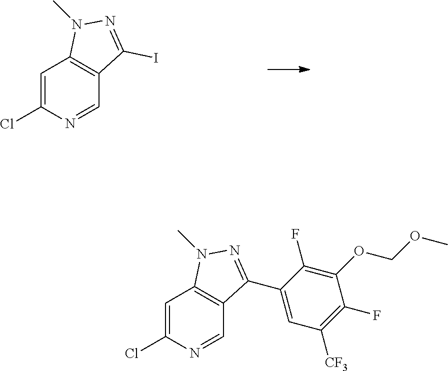 [SMILES: Cn1nc(I)c2cnc(Cl)cc21 ; COCOc1c(F)c(-c2nn(C)c3cc(Cl)ncc23)cc(C(F)(F)F)c1F]

A mixture of Intermediate 17.09 (0.15 g, 0.51 mmol), Intermediate 11 (0.23 g, 0.61 mmol), Pd(dppf)Cl₂ (37 mg, 0.051 mmol), KF (0.12 g, 2.04 mmol), dioxane (2 mL), and water (0.5 mL) was purged with nitrogen for 5 min, heated in a microwave at 90° C. for 30 mi and then partitioned between DCM and saturated NaHC₃ solution. The aqueous layer was separated and extracted with DCM. The combined organics were washed (saturated NaHCO₃), dried (Na₂SO₄), and concentrated. The residue was purified by silica gel chromatography (0-40% EtOAc/hexanes) to give 6-chloro-3-(2,4-difluoro-3-(methoxymethoxy)-5-(trifluoromethyl)phenyl)-1-methyl-1H-pyrazolo[4,3-c]pyridine(0.18 g, 85%) as a white powder. ¹H NMR (400 MHz, DMSO-d₆): δ 8.99 (d, 1H), 8.02 (s, 1H), 7.92 (t, 1H), 5.34 (s, 2H), 4.15 (s, 3H), 3.56 (s, 3H); LCMS: 408.0 [M+H]⁺.

The Intermediates below were synthesized in a similar manner to that described for Intermediate 18.

| Int | Structure | Name | [M + H]⁺ |
|---|---|---|---|
| 18 (3) | 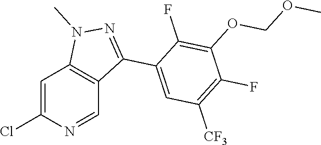 [SMILES: COCOc1c(F)c(-c2nn(C)c3cc(Cl)ncc23)cc(C(F)(F)F)c1F] | 6-Chloro-3-(2,4-difluoro-3- (methoxymethoxy)-5- (trifluoromethyl)phenyl)-1-methyl- 1H-pyrazolo[4,3-c]pyridine | 408.0 |
| 18.01 | 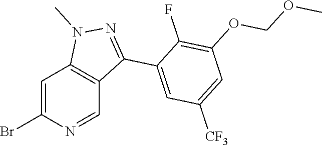 [SMILES: COCOc1cc(C(F)(F)F)cc(-c2nn(C)c3cc(Br)ncc23)c1F] | 6-Bromo-3-(2-fluoro-3- (methoxymethoxy)-5- (trifluoromethyl)phenyl)-1-methyl- 1H-pyrazolo[4,3-c]pyridine | 434.1 |
| 18.02 | 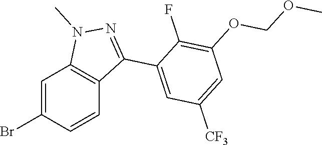 [SMILES: COCOc1cc(C(F)(F)F)cc(-c2nn(C)c3cc(Br)ccc23)c1F] | 6-Bromo-3-(2-fluoro-3- (methoxymethoxy)-5- (trifluoromethyl)phenyl)-1-methyl- 1H-indazole | 433.1 |
| 18.03 | 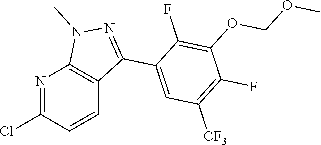 [SMILES: COCOc1c(F)c(-c2nn(C)c3nc(Cl)ccc23)cc(C(F)(F)F)c1F] | 6-Chloro-3-(2,4-difluoro-3- (methoxymethoxy)-5- (trifluoromethyl)phenyl)-1-methyl- 1H-pyrazolo[3,4-b]pyridine | 407.9 |
| 18.04 | 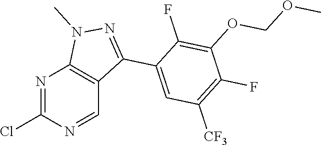 [SMILES: COCOc1c(F)c(-c2nn(C)c3nc(Cl)ncc23)cc(C(F)(F)F)c1F] | 6-Chloro-3-(2,4-difluoro-3- (methoxymethoxy)-5- (trifluoromethyl)phenyl)-1-methyl- 1H-pyrazolo[3,4-d]pyrimidine | 409.0 |
| 18.05 (1) | 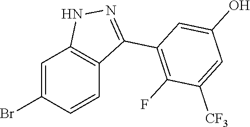 [SMILES: Oc1cc(-c2n[nH]c3cc(Br)ccc23)c(F)c(C(F)(F)F)c1] | 3-(6-Bromo-1H-indazol-3-yl)-4- fluoro-5-(trifluoromethyl)phenol | 374.8 |
| 18.06 (2) | 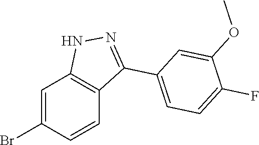 [SMILES: COc1cc(-c2n[nH]c3cc(Br)ccc23)ccc1F] | 6-Bromo-3-(4-fluoro-3- methoxyphenyl)-1H-indazole | ¹H NMR |
| 18.07 (2) | 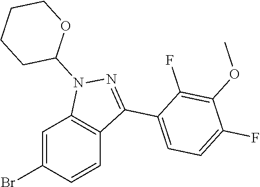 [SMILES: COc1c(F)ccc(-c2nn(C3CCCCO3)c3cc(Br)ccc23)c1F] | 6-Bromo-3-(2,4-difluoro-3- methoxyphenyl)-1-(tetrahydro-2H- pyran-2-yl)-1H-indazole | 423.0 |
| 18.08 (2) | 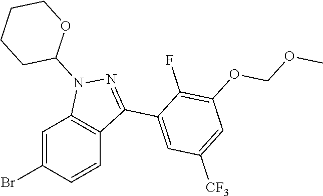 [SMILES: COCOc1cc(C(F)(F)F)cc(-c2nn(C3CCCCO3)c3cc(Br)ccc23)c1F] | 6-Bromo-3-(2-fluoro-3- (methoxymethoxy)-5- (trifluoromethyl)phenyl)-1- (tetrahydro-2H-pyran-2-yl)-1H- indazole | 503.1 |
| 18.09 (2) | 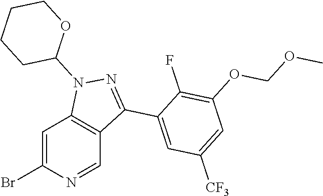 [SMILES: COCOc1cc(C(F)(F)F)cc(-c2nn(C3CCCCO3)c3cc(Br)ncc23)c1F] | 6-Bromo-3-(2-fluoro-3- (methoxymethoxy)-5- (trifluoromethyl)phenyl)-1- (tetrahydro-2H-pyran-2-yl)-1H- pyrazolo[4,3-c]pyridine | 504.1 |
| 18.10 | 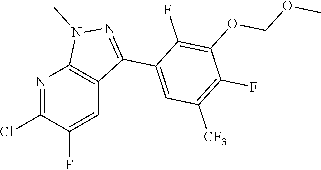 [SMILES: COCOc1c(F)c(-c2nn(C)c3nc(Cl)c(F)cc23)cc(C(F)(F)F)c1F] | 6-Chloro-3-(2,4-difluoro-3- (methoxymethoxy)-5- (trifluoromethyl)phenyl)-5-fluoro-1- methyl-1H-pyrazolo[3,4-b]pyridine | 426.0 |
| 18.11 | 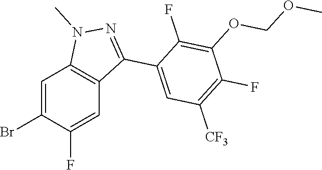 [SMILES: COCOc1c(F)c(-c2nn(C)c3cc(Br)c(F)cc23)cc(C(F)(F)F)c1F] | 6-Bromo-3-(2,4-difluoro-3- (methoxymethoxy)-5- (trifluoromethyl)phenyl)-5-fluoro-1- methyl-1H-indazole | 469.2 |
| 18.12 (3) | 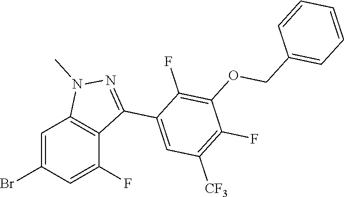 [SMILES: Cn1nc(-c2cc(C(F)(F)F)c(F)c(OCc3ccccc3)c2F)c2c(F)cc(Br)cc21] | 3-(3-(Benzyloxy)-2,4-difluoro-5- (trifluoromethyl)phenyl)-6-bromo-4- fluoro-1-methyl-1H-indazole | 515.0 |
| 18.13 (4) | 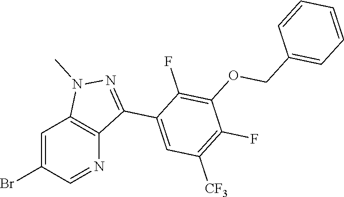 [SMILES: Cn1nc(-c2cc(C(F)(F)F)c(F)c(OCc3ccccc3)c2F)c2ncc(Br)cc21] | 3-(3-(Benzyloxy)-2,4-difluoro-5- (trifluoromethyl)phenyl)-6-bromo-1- methyl-1H-pyrazolo[4,3-b]pyridine | 498.0 |
| 18.14 (1) | 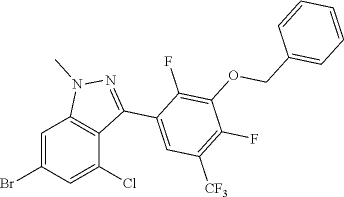 [SMILES: Cn1nc(-c2cc(C(F)(F)F)c(F)c(OCc3ccccc3)c2F)c2c(Cl)cc(Br)cc21] | 3-(3-(Benzyloxy)-2,4-difluoro-5- (trifluoromethyl)phenyl)-6-bromo-4- chloro-1-methyl-1H-indazole | 531.0 |
| 18.15 | 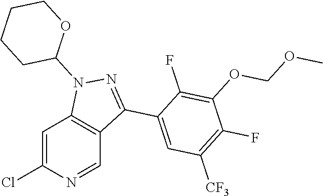 [SMILES: COCOc1c(F)c(-c2nn(C3CCCCO3)c3cc(Cl)ncc23)cc(C(F)(F)F)c1F] | 6-Chloro-3-(2,4-difluoro-3- (methoxymethoxy)-5- (trifluoromethyl)phenyl)-1- (tetrahydro-2H-pyran-2-yl)-1H- pyrazolo[4,3-c]pyridine | 478.1 |
| 18.16 | 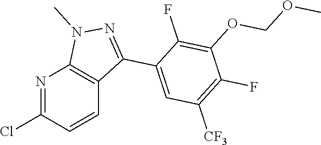 [SMILES: COCOc1c(F)c(-c2nn(C)c3nc(Cl)ccc23)cc(C(F)(F)F)c1F] | 6-Chloro-3-(2,4-difluoro-3- (methoxymethoxy)-5- (trifluoromethyl)phenyl)-1-methyl- 1H-pyrazolo[3,4-b]pyridine | 407.9 |
| 18.17 (5) | 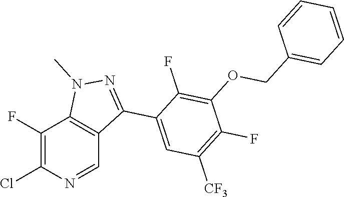 [SMILES: Cn1nc(-c2cc(C(F)(F)F)c(F)c(OCc3ccccc3)c2F)c2cnc(Cl)c(F)c21] | 3-(3-(Benzyloxy)-2,4-difluoro-5- (trifluoromethyl)phenyl)-6-chloro-7- fluoro-1-methyl-1H-pyrazolo[4,3- c]pyridine | 472.1 |
| 18.18 (5) | 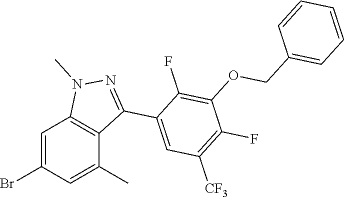 [SMILES: Cc1cc(Br)cc2c1c(-c1cc(C(F)(F)F)c(F)c(OCc3ccccc3)c1F)nn2C] | 3-(3-(Benzyloxy)-2,4-difluoro-5- (trifluoromethyl)phenyl)-6-bromo- 1,4-dimethyl-1H-indazole | 511.1 |
| 18.19 | 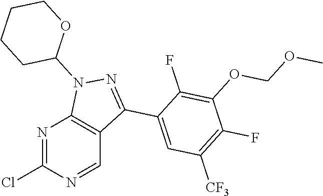 [SMILES: COCOc1c(F)c(-c2nn(C3CCCCO3)c3nc(Cl)ncc23)cc(C(F)(F)F)c1F] | 6-Chloro-3-(2,4-difluoro-3- (methoxymethoxy)-5- (trifluoromethyl)phenyl)-1- (tetrahydro-2H-pyran-2-yl)-1H- pyrazolo[3,4-d]pyrimidine | 479.3 |
| 18.20 | 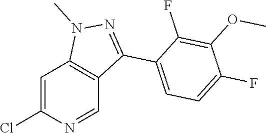 [SMILES: COc1c(F)ccc(-c2nn(C)c3cc(Cl)ncc23)c1F] | 6-Chloro-3-(2,4-difluoro-3- methoxyphenyl)-1-methyl-1H- pyrazolo[4,3-c]pyridine | 310.2 |
| 18.21 | 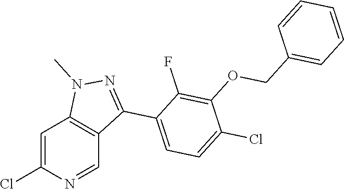 [SMILES: Cn1nc(-c2ccc(Cl)c(OCc3ccccc3)c2F)c2cnc(Cl)cc21] | 3-(3-(Benzyloxy)-4-chloro-2- fluorophenyl)-6-chloro-1-methyl-1H- pyrazolo[4,3-c]pyridine | 402.3 |
| 18.22 (3) | 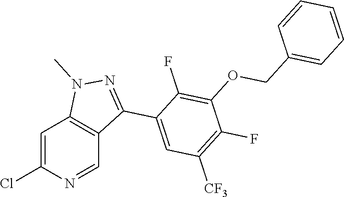 [SMILES: Cn1nc(-c2cc(C(F)(F)F)c(F)c(OCc3ccccc3)c2F)c2cnc(Cl)cc21] | 3-(3-(Benzyloxy)-2,4-difluoro-5- (trifluoromethyl)phenyl)-6-chloro-1- methyl-1H-pyrazolo[4,3-c]pyridine | 454.0 |
| 18.23 | 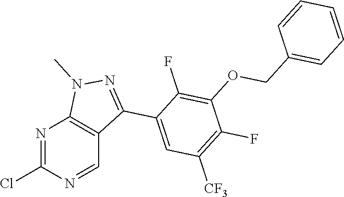 [SMILES: Cn1nc(-c2cc(C(F)(F)F)c(F)c(OCc3ccccc3)c2F)c2cnc(Cl)nc21] | 3-(3-(Benzyloxy)-2,4-difluoro-5- (trifluoromethyl)phenyl)-6-chloro-1- methyl-1H-pyrazolo[3,4- d]pyrimidine | 455.0 |

Alternate conditions used:
1. KF, Pd(dppf)Cl₂, dioxane:H₂O, 80-90° C.;
2. Pd(dppf)Cl₂, K₂CO₃, dioxane:H₂O, 85° C.;
3. Pd(dppf)Cl₂•CH₂Cl₂, 2 M Na₂CO₃, dioxane, 80° C., 1-2 h; 4. Pd(dppf)Cl₂, 1 M K₂CO₃, CH₃CN:H₂O, 80° C., overnight;
5. Pd(PPh₃)₄, K₂CO₃, dioxane:H₂O, 100° C., overnight. Intermediate 18.06: ¹H NMR (400 MHz, DMSO-d₆): δ 13.37 (s, 1H), 8.01 (d, 1H), 7.82 (d, 1H), 7.64 (d, 1H), 7.51-7.49 (m, 1H), 7.38-7.29 (m, 2H), 3.95 (s, 3H).

### Intermediate 19

### 3-(3-(Benzyloxy)-2,4-difluoro-5-(trifluoromethyl)phenyl)-6-chloro-1-methyl-1H-pyrazolo[4,3-c]pyridazine

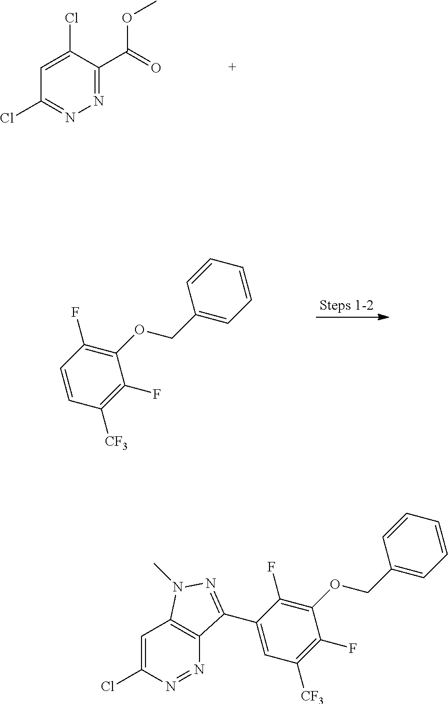 [SMILES: COC(=O)c1nnc(Cl)cc1Cl ; Fc1ccc(C(F)(F)F)c(F)c1OCc1ccccc1 ; Cn1nc(-c2cc(C(F)(F)F)c(F)c(OCc3ccccc3)c2F)c2nnc(Cl)cc21]

### Step 1: (3-(Benzyloxy)-2,4-difluoro-5-(trifluoromethyl)phenyl)(4,6-dichloropyridazin-3-yl)methanone

n-Butyllithium (2.5 M in toluene, 6.67 mL) was added dropwise to a mixture of Intermediate 12, Step 3 (4.59 g, 15.9 mmol) and THE (50 mL) at −78° C. under N₂. The mixture was stirred at −78° C. for 1 h. This mixture was added to a mixture of methyl 4,6-dichloropyridazine-3-carboxylate (3.0 g, 14.5 mmol) and THE (30 mL) at −78° C. under N₂. The mixture was stirred at −78° C. for 1 h, quenched with NH₄Cl (100 mL), and then extracted with EtOAc (3×40 mL). The combined organic layers were washed with brine (100 mL), dried over Na₂SO₄, filtered, concentrated, and then purified by column chromatography (SiO₂, petroleum ether/ethyl acetate=100/1 to 0/1) to give (3-(benzyloxy)-2,4-difluoro-5-(trifluoromethyl)phenyl)(4,6-dichloropyridazin-3-yl)methanone (2.2 g, 33%) as a yellow oil. ¹H NMR (400 MHz, DMSO-d₆) δ 8.71-8.66 (m, 1H), 7.99 (t, 1H), 7.46-7.34 (m, 5H), 5.28 (s, 2H); LCMS: 463.0 [M+H]⁺.

### Step 2: 3-(3-(Benzyloxy)-2,4-difluoro-5-(trifluoromethyl)phenyl)-6-chloro-1-methyl-1H-pyrazolo[4,3-c]pyridazine

Aqueous methylhydrazine (40%, 700 mg, 6.1 mmol) was added to a solution of (3-(benzyloxy)-2,4-difluoro-5-(trifluoromethyl)phenyl)(4,6-dichloropyridazin-3-yl)methanone (2.2 g, 4.75 mmol), DIPEA (1.84 g, 14.2 mmol), and MeOH (25 mL). The mixture was stirred at 55° C. for 10 min, allowed to cool to rt, and then filtered. The filter cake was concentrated under reduced pressure to give 3-(3-(benzyloxy)-2,4-difluoro-5-(trifluoromethyl)phenyl)-6-chloro-1-methyl-1H-pyrazolo[4,3-c]pyridazine (1.1 g, 51%) as a white solid. ¹H NMR (400 MHz, DMSO-d₆): δ 8.55 (t, 1H), 8.52 (s, 1H), 7.53-7.48 (m, 2H), 7.45-7.36 (m, 3H), 5.35 (s, 2H), 4.17 (s, 3H); LCMS: 455.1 [M+H]⁺.

### Intermediate 20

### 5-(6-Bromo-1H-indazol-3-yl)-2-fluoro-3-(trifluoromethyl)phenol

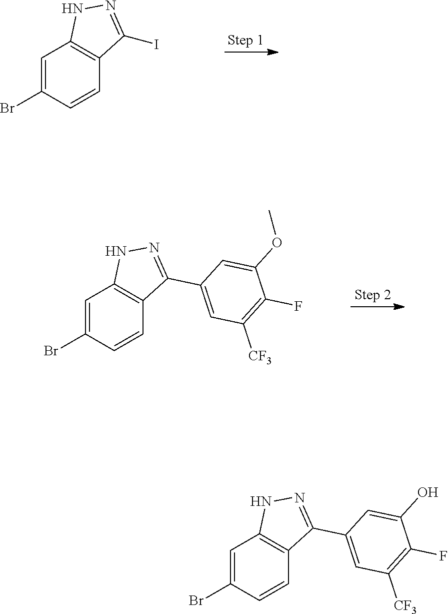 [SMILES: Brc1ccc2c(I)n[nH]c2c1 ; COc1cc(-c2n[nH]c3cc(Br)ccc23)cc(C(F)(F)F)c1F ; Oc1cc(-c2n[nH]c3cc(Br)ccc23)cc(C(F)(F)F)c1F]

### Step 1: 6-Bromo-3-(4-fluoro-3-methoxy-5-(trifluoromethyl)phenyl)-1H-indazole

A mixture of 6-bromo-3-iodo-1H-indazole (3.00 g, 9.29 mmol), 4-fluoro-3-methoxy-5-(trifluoromethyl)phenylboronic acid (3.32 g, 13.9 mmol), KF (1.62 g, 27.9 mmol), and Pd(dppf)Cl₂ (0.68 g, 0.93 mmol) in dioxane (4 mL) and water (1 mL) was purged with nitrogen for 5 min, heated in a microwave at 90° C. for 40 min, diluted with water, and then extracted with EtOAc. The combined organics were dried (Na₂SO₄) and concentrated under reduced pressure. The residue was purified by silica gel chromatography (0-100% EtOAc/hexanes) to yield 6-bromo-3-(4-fluoro-3-methoxy-5-(trifluoromethyl)phenyl)-1H-indazole (1.90 g, 53%) as a white powder. LCMS: 390.8 [M+H]⁺.

### Step 2: 5-(6-Bromo-1H-indazol-3-yl)-2-fluoro-3-(trifluoromethyl)phenol

A mixture of 6-bromo-3-(4-fluoro-3-methoxy-5-(trifluoromethyl)phenyl)-1H-indazole (500 mg, 1.28 mmol) and pyridine hydrochloride (743 mg, 6.42 mmol) was heated at 150° C. for 15 h, cooled to rt, diluted with water, and then extracted with EtOAc. The organics were dried (Na₂SO₄) and concentrated under reduced pressure. The residue was purified by silica gel chromatography (0-100% EtOAc/hexanes) to give 5-(6-bromo-1H-indazol-3-yl)-2-fluoro-3-(trifluoromethyl)phenol (423 mg, 88%) as a white powder. ¹H NMR (400 MHz, DMSO-d₆): δ 13.52 (s, 1H), 10.87 (s, 1H), 7.94 (d, J=8.8 Hz, 1H), 7.90-7.83 (m, 2H), 7.61 (br d, J=5.1 Hz, 1H), 7.39 (d, J=8.7 Hz, 1H); LCMS: 376.7 [M+H]⁺.

The Intermediates below were synthesized in a similar manner to that described for Intermediate 20.

| Int | Structure | Name | [M + H]⁺ |
|---|---|---|---|
| 20.01 | 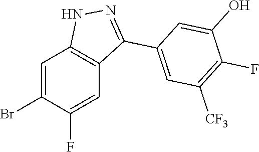 [SMILES: Oc1cc(-c2n[nH]c3cc(Br)c(F)cc23)cc(C(F)(F)F)c1F] | 5-(6-Bromo-5-fluoro-1H-indazol-3- yl)-2-fluoro-3- (trifluoromethyl)phenol | 392.8 |
| 20.02 | 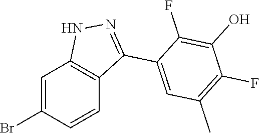 [SMILES: Cc1cc(-c2n[nH]c3cc(Br)ccc23)c(F)c(O)c1F] | 3-(6-Bromo-1H-indazol-3-yl)-2,6- difluoro-5-methylphenol | 338.8 |
| 20.03 | 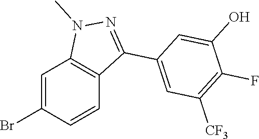 [SMILES: Cn1nc(-c2cc(O)c(F)c(C(F)(F)F)c2)c2ccc(Br)cc21] | 5-(6-Bromo-1-methyl-1H-indazol-3- yl)-2-fluoro-3- (trifluoromethyl)phenol | 388.9 |
| 20.04 | 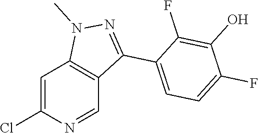 [SMILES: Cn1nc(-c2ccc(F)c(O)c2F)c2cnc(Cl)cc21] | 3-(6-Chloro-1-methyl-1H- pyrazolo[4,3-c]pyridin-3-yl)-2,6- difluorophenol | 295.9 |

### Intermediate 21

### 3-(6-Chloro-1,7-dimethyl-1H-pyrazolo[4,3-c]pyridin-3-yl)-2,6-difluoro-5-(trifluoromethyl)phenol

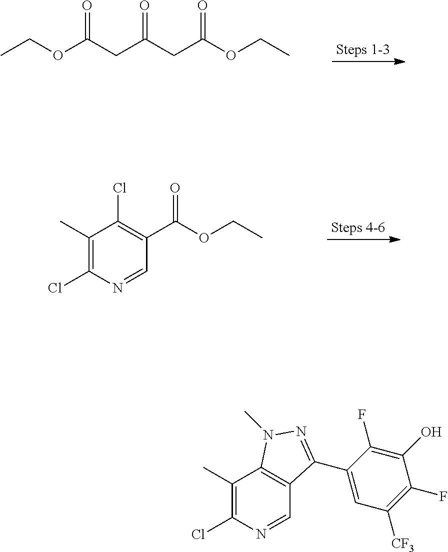

### Step 1: Diethyl 2-methyl-3-oxopentanedioate

Sodium hydride (10.9 g, 272 mmol, 60% purity) was added to a solution of diethyl 3-oxopentanedioate (50.1 g, 247.28 mmol) in THF (500 mL) at 0° C. under N₂. The mixture was stirred at 0° C. for 1 h. Iodomethane (16.9 mL, 272 mmol) was added dropwise at 0° C. The reaction mixture was stirred at rt for 24 h and then carefully poured into sat. aq. NH₄Cl (500 mL). The reaction was stirred for 30 min at 0° C. The aqueous phase was extracted with ethyl acetate (3×200 mL). The combined organic phases were washed with brine (500 mL), dried (Na₂SO₄), filtered, concentrated, and then purified by silica gel chromatography (petroleum ether/ethyl acetate=50:1 to 5:1) to give diethyl 2-methyl-3-oxopentanedioate (13.2 g, 24%) as a yellow oil. ¹H NMR (400 MHz, DMSO-d₆): δ 4.11-4.08 (m, 4H), 3.87-3.81 (m, 1H), 3.73 (s, 2H), 1.23-1.14 (m, 9H); LCMS: 217.2 [M+H]⁺.

### Step 2: Ethyl 4,6-dihydroxy-5-methylnicotinate

A mixture of diethyl 2-methyl-3-oxopentanedioate (10 g, 46.3 mmol), triethoxymethane (7.54 g, 50.9 mmol), and Ac₂O (8.66 mL, 92.5 mmol) was stirred at 130° C. for 2 h under N₂, allowed to cool to rt, and then concentrated under reduced pressure to give a yellow oil. The residue was diluted with NH₃·H₂O (4.2 mL, 32.4 mmol, 30% purity) dropwise at 0° C., and the resulting mixture was stirred at rt for 2 h. Water (5 mL) was added into the mixture. The mixture was adjusted to pH=˜5 with 2 M HCl, stirred at rt for 0.5 h, and then filtered. The filter cake was dried and then triturated in PE (20 mL) to give ethyl 4,6-dihydroxy-5-methylnicotinate (4.4 g, 48%) as a yellow solid. ¹H NMR (400 MHz, CDCl₃-d): δ 13.48 (s, 1H), 11.05 (s, 1H), 8.20 (s, 1H), 4.41-4.36 (m, 2H), 2.05 (s, 3H), 1.42 (t, 3H); LCMS: 198.1 [M+H]⁺.

### Step 3: Ethyl 4,6-dichloro-5-methylnicotinate

A mixture of ethyl 4,6-dihydroxy-5-methylnicotinate (4.0 g, 20.3 mmol) in POCl₃ (24 mL, 258 mmol) was stirred at 120° C. for 3 h, allowed to cool to rt, poured into water (500 mL) carefully, and then adjusted to pH=˜7 with Na₂CO₃. The aqueous phase was extracted with ethyl acetate (3×300 mL). The combined organic phases were washed with brine (300 mL), dried (Na₂SO₄), filtered, concentrated, and then purified by silica gel chromatography (petroleum ether/ethyl acetate=50:1 to 5:1) to give ethyl 4,6-dichloro-5-methylnicotinate (4.1 g, 86%) as a colorless oil. ¹H NMR (400 MHz, CDCl₃-d): δ 8.61 (s, 1H), 4.44-4.21 (m, 2H), 2.55 (s, 3H), 1.41 (t, 3H); LCMS: 234.1 [M+H]⁺.

### Step 4: (3-(Benzyloxy)-2,4-difluoro-5-(trifluoromethyl)phenyl)(4,6-dichloro-5-methylpyridin-3-yl)methanone

n-Butyllithium (2.5 M in hexane, 6.7 mL, 16.7 mmol) was added dropwise to a mixture of 2-(benzyloxy)-1,3-difluoro-4-(trifluoromethyl)benzene (4.43 g, 15.4 mmol) in THF (40 mL) at −78° C. under N₂. The mixture was stirred at −78° C. for 1 h. Ethyl 4,6-dichloro-5-methylnicotinate (3 g, 12.8 mmol) in THF (30 mL) was added. The mixture was stirred for 1 h, quenched with sat. aq. NH₄Cl (200 mL), and then extracted with EtOAc (3×100 mL). The combined organic layers were washed with brine (100 mL), dried (Na₂SO₄), filtered, and then concentrated to give (3-(benzyloxy)-2,4-difluoro-5-(trifluoromethyl)phenyl)(4,6-dichloro-5-methylpyridin-3-yl)methanone (6 g) as a colorless oil. ¹H NMR (400 MHz, DMSO-d₆): δ 8.47 (s, 1H), 7.86 (t, 1H), 7.44-7.37 (m, 5H), 5.26 (s, 2H), 2.48 (s, 3H); LCMS: 476.0 [M+H]⁺.

### Step 5: (4,6-Dichloro-5-methylpyridin-3-yl)(2,4-difluoro-3-hydroxy-5-(trifluoromethyl)phenyl)methanone

A mixture of (3-(benzyloxy)-2,4-difluoro-5-(trifluoromethyl)phenyl)(4,6-dichloro-5-methylpyridin-3-yl)methanone (6.01 g, 12.6 mmol) in TFA (40 mL) was stirred at 70° C. for 2 h, allowed to cool to rt, adjusted to pH=˜7 with sat. aq. NaHCO₃, and then extracted with DCM (3×50 mL). The combined organic layers were washed with brine (50 mL), dried (Na₂SO₄), filtered, concentrated, and then purified by silica gel chromatography (petroleum ether/ethyl acetate=30:1 to 3:1) to give (4,6-dichloro-5-methylpyridin-3-yl)(2,4-difluoro-3-hydroxy-5-(trifluoromethyl)phenyl)methanone (3.3 g, 67%) as a yellow solid. ¹H NMR (400 MHz, DMSO-d₆): δ 11.71 (s, 1H), 8.48 (s, 1H), 7.55 (t, 1H), 2.48 (s, 3H); LCMS: 386.0 [M+H]⁺.

### Step 6: 3-(6-Chloro-1,7-dimethyl-1H-pyrazolo[4,3-c]pyridin-3-yl)-2,6-difluoro-5-(trifluoromethyl)phenol

Methylhydrazine (0.82 mL, 6.22 mmol, 40% purity) was added to a solution of (4,6-dichloro-5-methylpyridin-3-yl)(2,4-difluoro-3-hydroxy-5-(trifluoromethyl)phenyl)methanone (2 g, 5.18 mmol) and DIEA (2.7 mL, 15.5 mmol) in MeOH (20 mL) at rt. The mixture was stirred at 55° C. for 12 h, allowed to cool to rt, concentrated, and then purified by reverse-phase HPLC [water (10 mM NH₄HCO₃, NH₃·H₂O)/CH₃CN] to give 3-(6-chloro-1,7-dimethyl-1H-pyrazolo[4,3-c]pyridin-3-yl)-2,6-difluoro-5-(trifluoromethyl)phenol (1.3 g, 66%) as a yellow solid. ¹H NMR (400 MHz, DMSO-d₆): δ 11.29 (s, 1H), 8.74 (d, 1H), 7.47 (t, 1H), 4.31 (s, 3H), 2.81 (s, 3H); LCMS: 378.0 [M+H]⁺.

### Intermediate 22

### 7-Chloro-3-iodo-N,1-dimethyl-N-(tetrahydro-2H-pyran-4-yl)-1H-pyrazolo[4,3-c]pyridin-6-amine

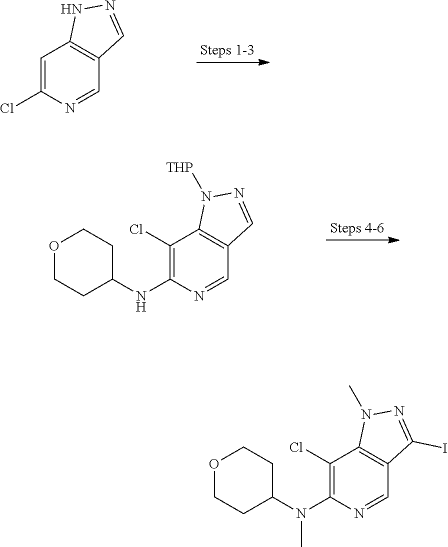

### Step 1: 6-Chloro-1-(tetrahydro-2H-pyran-2-yl)-1H-pyrazolo[4,3-c]pyridine

A mixture of 6-chloro-1H-pyrazolo[4,3-c]pyridine (3 g, 19.54 mmol), DHP (5.4 mL, 58.61 mmol), and TsOH·H₂O (743 mg, 3.91 mmol) in DCM (40 mL) was stirred at 45° C. for 16 h, allowed to cool to rt, adjusted to pH=7 with sat. aq. NaHCO₃, and then extracted with DCM (3×30 mL). The combined organic layers were washed with brine (30 mL), dried (Na₂SO₄), filtered, concentrated, and then purified by silica gel chromatography (petroleum ether/ethyl acetate=30:1 to 3:1) to give 6-chloro-1-(tetrahydro-2H-pyran-2-yl)-1H-pyrazolo[4,3-c]pyridine (2.8 g, 60%) as a yellow solid. ¹H NMR (400 MHz, DMSO-d₆): δ 8.95 (d, 1H), 8.38 (s, 1H), 7.93 (s, 1H), 5.91 (dd, 1H), 3.90-3.87 (m, 1H), 3.79-3.73 (m, 1H), 2.36-2.32 (m, 1H), 2.03-1.95 (m, 2H), 1.73-1.69 (m, 1H), 1.60-1.55 (m, 2H); LCMS: 238.1 [M+H]⁺.

### Step 2: N-Methyl-1-(tetrahydro-2H-pyran-2-yl)-N-(tetrahydro-2H-pyran-4-yl)-1H-pyrazolo[4,3-c]pyridin-6-amine

Tris(dibenzylideneacetone)dipalladium(0) (771 mg, 0.841 mmol) was added to a mixture of 6-chloro-1-(tetrahydro-2H-pyran-2-yl)-1H-pyrazolo[4,3-c]pyridine (2 g, 8.41 mmol), N-methyltetrahydro-2H-pyran-4-amine (1.45 g, 12.6 mmol), RuPhos (785 mg, 1.68 mmol), and NaOtBu (1.62 g, 16.8 mmol) in dioxane (30 mL) under N₂. The mixture was degassed and purged with N₂ 3 times, stirred at 100° C. for 2 h, allowed to cool to rt, poured into water (50 mL), and then extracted with ethyl acetate (3×30 mL). The combined organic layers were washed with brine (30 mL), dried (Na₂SO₄), filtered, concentrated, and then purified by silica gel chromatography (petroleum ether/ethyl acetate=10:1 to 1:1) to give N-methyl-1-(tetrahydro-2H-pyran-2-yl)-N-(tetrahydro-2H-pyran-4-yl)-1H-pyrazolo[4,3-c]pyridin-6-amine (1.6 g, 60%) as a yellow oil. ¹H NMR (400 MHz, DMSO-d₆): δ 8.65 (s, 1H), 8.01 (s, 1H), 6.53 (s, 1H), 5.75-5.73 (m, 1H), 4.98-4.92 (m, 1H), 3.96-3.92 (m, 2H), 3.88-3.85 (m, 1H), 3.76-3.69 (m, 1H), 3.44 (t, 2H), 2.86 (s, 3H), 2.38-2.33 (m, 1H), 1.91-1.83 (m, 2H), 1.82-1.68 (m, 3H), 1.57-1.46 (m, 4H); LCMS: 317.2 [M+H]⁺.

### Step 3: 7-Chloro-N-methyl-1-(tetrahydro-2H-pyran-2-yl)-N-(tetrahydro-2H-pyran-4-yl)-1H-pyrazolo[4,3-c]pyridin-6-amine

A mixture of N-methyl-1-(tetrahydro-2H-pyran-2-yl)-N-(tetrahydro-2H-pyran-4-yl)-1H-pyrazolo[4,3-c]pyridin-6-amine (1.6 g, 5.06 mmol) and NCS (878 mg, 6.57 mmol) in MeCN (20 mL) was stirred at 60° C. for 2 h. The reaction mixture was cooled to rt slowly and then concentrated to dryness to give 7-chloro-N-methyl-1-(tetrahydro-2H-pyran-2-yl)-N-(tetrahydro-2H-pyran-4-yl)-1H-pyrazolo[4,3-c]pyridin-6-amine (1.9 g) as a yellow oil. ¹H NMR (400 MHz, DMSO-d₆): δ 8.76 (s, 1H), 8.28 (s, 1H), 6.23 (d, 1H), 3.94-3.88 (m, 4H), 3.74-3.66 (m, 2H), 3.35-3.30 (m, 4H), 2.91 (s, 3H), 1.82-1.71 (m, 2H), 1.69-1.61 (m, 3H), 1.56-1.54 (m, 2H); LCMS: 351.2 [M+H]⁺.

### Step 4: 7-Chloro-N-methyl-N-(tetrahydro-2H-pyran-4-yl)-1H-pyrazolo[4,3-c]pyridin-6-amine

A mixture of 7-chloro-N-methyl-1-(tetrahydro-2H-pyran-2-yl)-N-(tetrahydro-2H-pyran-4-yl)-1H-pyrazolo[4,3-c]pyridin-6-amine (1.9 g, 5.42 mmol) in TFA (8 mL) and DCM (12 mL) was stirred at rt for 4 h. The reaction mixture was concentrated under reduced pressure, adjusted to pH=7 with sat. aq. NaHCO₃, and then extracted with DCM (3×30 mL). The combined organic layers were washed with brine (30 mL), dried (Na₂SO₄), filtered, concentrated, and then purified by silica gel chromatography (petroleum ether/ethyl acetate=10:1 to 1:1) to give 7-chloro-N-methyl-N-(tetrahydro-2H-pyran-4-yl)-1H-pyrazolo[4,3-c]pyridin-6-amine (980 mg, 67%) as a yellow solid. ¹H NMR (400 MHz, DMSO-d₆): δ 13.53 (s, 1H), 8.73 (s, 1H), 8.22 (s, 1H), 3.91-3.87 (m, 2H), 3.82-3.76 (m, 1H), 3.34-3.29 (m, 2H), 2.82 (s, 3H), 1.84-1.74 (m, 2H), 1.65-1.62 (m, 2H); LCMS: 267.1 [M+H]⁺.

### Step 5: 7-Chloro-3-iodo-N-methyl-N-(tetrahydro-2H-pyran-4-yl)-1H-pyrazolo[4,3-c]pyridin-6-amine

Iodine (1.67 g, 6.60 mmol) and KOH (555 mg, 9.90 mmol) were added to a mixture of 7-chloro-N-methyl-N-(tetrahydro-2H-pyran-4-yl)-1H-pyrazolo[4,3-c]pyridin-6-amine (0.88 g, 3.30 mmol) in DMF (10 mL) at 0° C. The mixture was stirred at rt for 3 h, poured into sat. aq. Na₂SO₃ (20 mL), and then extracted with EtOAc (3×10 mL). The combined organic layers were washed with brine (20 mL), dried (Na₂SO₄), filtered, concentrated, and then purified by silica gel chromatography (petroleum ether/ethyl acetate=30:1 to 3:1) to give 7-chloro-3-iodo-N-methyl-N-(tetrahydro-2H-pyran-4-yl)-1H-pyrazolo[4,3-c]pyridin-6-amine (820 mg, 63%) as a yellow solid. ¹H NMR (400 MHz, DMSO-d₆): δ 13.89 (s, 1H), 8.40 (s, 1H), 3.92-3.88 (m, 3H), 3.32 (t, 2H), 2.84 (s, 3H), 1.83-1.79 (m, 2H), 1.65-1.62 (m, 2H); LCMS: 393.0 [M+H]⁺.

### Step 6: 7-Chloro-3-iodo-N,1-dimethyl-N-(tetrahydro-2H-pyran-4-yl)-1H-pyrazolo[4,3-c]pyridin-6-amine

Iodomethane (287 mg, 2.02 mmol) was added to a mixture of 7-chloro-3-iodo-N-methyl-N-(tetrahydro-2H-pyran-4-yl)-1H-pyrazolo[4,3-c]pyridin-6-amine (795 mg, 2.02 mmol) and K₂CO₃ (280 mg, 2.02 mmol) in DMF (10 mL) at 0° C. The mixture was stirred at rt for 14 h, poured into water (50 mL), and then extracted with EtOAc (3×30 mL). The combined organic layers were washed with brine (30 mL), dried (Na₂SO₄), filtered, concentrated, and then purified by silica gel chromatography (petroleum ether/ethyl acetate=30:1 to 3:1) to give 7-chloro-3-iodo-N,1-dimethyl-N-(tetrahydro-2H-pyran-4-yl)-1H-pyrazolo[4,3-c]pyridin-6-amine (410 mg, 49%) as a yellow solid. ¹H NMR (400 MHz, DMSO-d₆): δ 8.38 (s, 1H), 4.24 (s, 3H), 3.92-3.88 (m, 2H), 3.78-3.70 (m, 1H), 3.34 (t, 2H), 2.81 (s, 3H), 1.86-1.74 (m, 2H), 1.65-1.60 (m, 2H); LCMS: 406.9 [M+H]⁺.

### Compound 1

### N-Cyclobutyl-2-(3,4-difluoro-5-hydroxyphenyl)benzo[d]oxazole-5-carboxamide

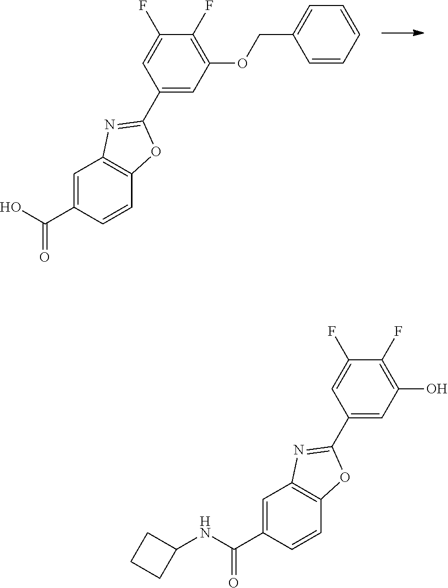 [SMILES: O=C(O)c1ccc2oc(-c3cc(F)c(F)c(OCc4ccccc4)c3)nc2c1 ; O=C(NC1CCC1)c1ccc2oc(-c3cc(O)c(F)c(F)c3)nc2c1]

DIEA (0.1 mL, 0.57 mmol) was added to a solution of Intermediate 6 (100 mg, 0.26 mmol), HATU (122 mg, 0.32 mmol), and DMF (2 mL) at rt. After stirring the mixture for 10 min, cyclobutylamine (0.10 mL, 0.52 mmol) was added. The mixture was stirred for 30 min, diluted (4 mL water), and then stirred for 30 min. The precipitate was collected by filtration and air dried overnight. The solid was taken up in THF (12 mL). Pd/C (1000, 20 mg) was added. The reaction was stirred under a balloon of hydrogen for 30 min and filtered. The filter cake was washed with THF, and the filtrate was concentrated. The residue was triturated in acetonitrile to give N-cyclobutyl-2-(3,4-difluoro-5-hydroxyphenyl)benzo[d]oxazole-5-carboxamide (55 mg, 61%) as a white solid. ¹H NMR (400 MHz, DMSO-d₆): δ 11.14 (s, 1H), 8.74 (d, J=7.5 Hz, 1H), 8.32-8.29 (m, 1H), 7.98-7.94 (m, 1H), 7.86 (d, J=8.6 Hz, 1H), 7.68-7.60 (m, 2H), 4.50-4.40 (m, 1H), 2.29-2.20 (m, 2H), 2.16-2.05 (m, 2H), 1.78-1.64 (m, 2H); LCMS: 345.0 [M+H]⁺.

The Compounds below were synthesized in a similar manner to that described for Compound 1.

| Cmpd | Structure | Name | [M + H]⁺ |
|---|---|---|---|
| 1.01 | 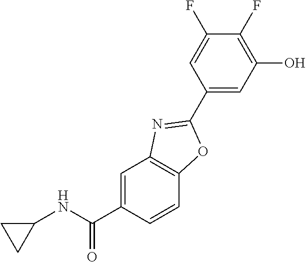 [SMILES: O=C(NC1CC1)c1ccc2oc(-c3cc(O)c(F)c(F)c3)nc2c1] | N-Cyclopropyl-2-(3,4-difluoro-5- hydroxyphenyl)benzo[d]oxazole-5- carboxamide | 330.9 |
| 1.02 | 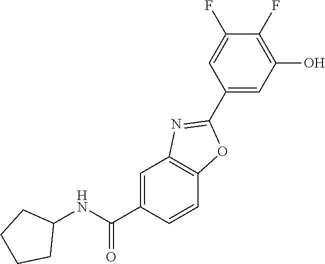 [SMILES: O=C(NC1CCCC1)c1ccc2oc(-c3cc(O)c(F)c(F)c3)nc2c1] | N-Cyclopentyl-2-(3,4-difluoro-5- hydroxyphenyl)benzo[d]oxazole-5- carboxamide | 359.0 |
| 1.03 | 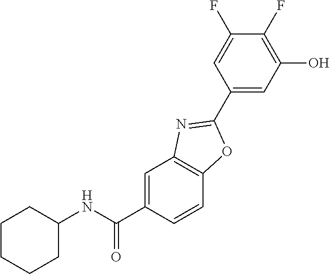 [SMILES: O=C(NC1CCCCC1)c1ccc2oc(-c3cc(O)c(F)c(F)c3)nc2c1] | N-Cyclohexyl-2-(3,4-difluoro-5- hydroxyphenyl)benzo[d]oxazole-5- carboxamide | 373.0 |
| 1.04 | 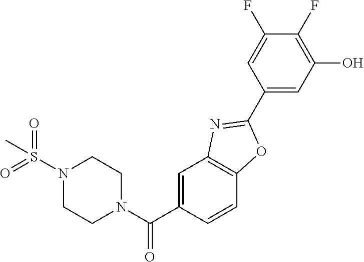 [SMILES: CS(=O)(=O)N1CCN(C(=O)c2ccc3oc(-c4cc(O)c(F)c(F)c4)nc3c2)CC1] | (2-(3,4-Difluoro-5- hydroxyphenyl)benzo[d]oxazol-5- yl)(4-(methylsulfonyl)piperazin-1- yl)methanone | 437.9 |
| 1.05 | 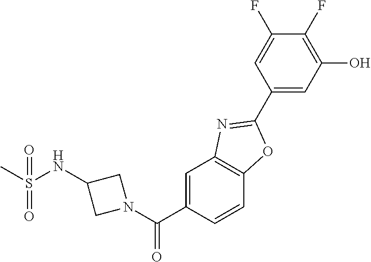 [SMILES: CS(=O)(=O)NC1CN(C(=O)c2ccc3oc(-c4cc(O)c(F)c(F)c4)nc3c2)C1] | N-(1-(2-(3,4-Difluoro-5- hydroxyphenyl)benzo[d]oxazole-5- carbonyl)azetidin-3- yl)methanesulfonamide | 424.0 |
| 1.06 | 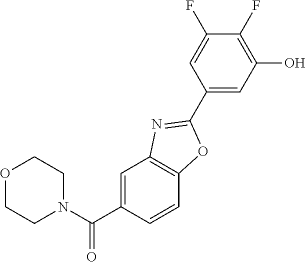 [SMILES: O=C(c1ccc2oc(-c3cc(O)c(F)c(F)c3)nc2c1)N1CCOCC1] | (2-(3,4-Difluoro-5- hydroxyphenyl)benzo[d]oxazol-5- yl)(morpholino)methanone | 361.0 |
| 1.07 | 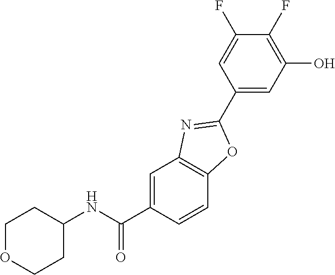 [SMILES: O=C(NC1CCOCC1)c1ccc2oc(-c3cc(O)c(F)c(F)c3)nc2c1] | 2-(3,4-Difluoro-5-hydroxyphenyl)-N- (tetrahydro-2H-pyran-4- yl)benzo[d]oxazole-5-carboxamide | 375.1 |
| 1.08 | 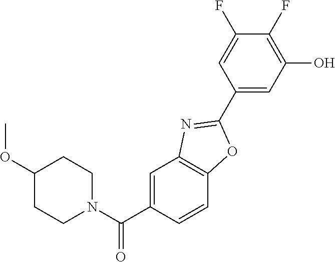 [SMILES: COC1CCN(C(=O)c2ccc3oc(-c4cc(O)c(F)c(F)c4)nc3c2)CC1] | (2-(3,4-Difluoro-5- hydroxyphenyl)benzo[d]oxazol-5- yl)(4-methoxypiperidin-1- yl)methanone | 389.0 |
| 1.09 | 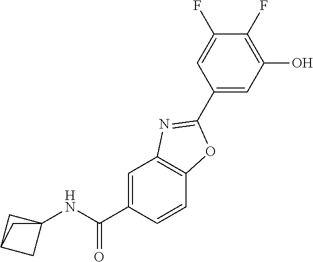 [SMILES: O=C(NC12CC(C1)C2)c1ccc2oc(-c3cc(O)c(F)c(F)c3)nc2c1] | N-(Bicyclo[1.1.1]pentan-1-yl)-2-(3,4- difluoro-5- hydroxyphenyl)benzo[d]oxazole-5- carboxamide | 357.0 |
| 1.10 | 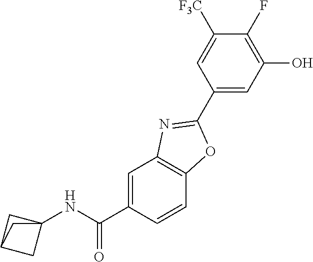 [SMILES: O=C(NC12CC(C1)C2)c1ccc2oc(-c3cc(O)c(F)c(C(F)(F)F)c3)nc2c1] | N-(Bicyclo[1.1.1]pentan-1-yl)-2-(4- fluoro-3-hydroxy-5- (trifluoromethyl)phenyl)benzo[d]oxa- zole-5-carboxamide | 407.0 |
| 1.11 | 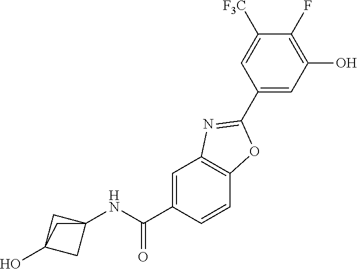 [SMILES: O=C(NC12CC(O)(C1)C2)c1ccc2oc(-c3cc(O)c(F)c(C(F)(F)F)c3)nc2c1] | 2-(4-Fluoro-3-hydroxy-5- (trifluoromethyl)phenyl)-N-(3- hydroxybicyclo[1.1.1]pentan-1- yl)benzo[d]oxazole-5-carboxamide | 423.0 |
| 1.12 (1) | 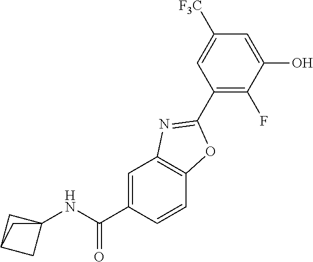 [SMILES: O=C(NC12CC(C1)C2)c1ccc2oc(-c3cc(C(F)(F)F)cc(O)c3F)nc2c1] | N-(Bicyclo[1.1.1]pentan-1-yl)-2-(2- fluoro-3-hydroxy-5- (trifluoromethyl)phenyl)benzo[d]oxa- zole-5-carboxamide | 407.0 |
| 1.13 (1) | 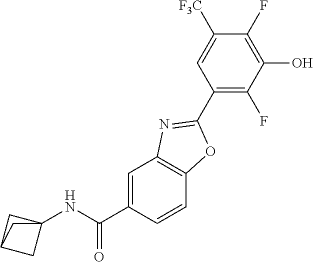 [SMILES: O=C(NC12CC(C1)C2)c1ccc2oc(-c3cc(C(F)(F)F)c(F)c(O)c3F)nc2c1] | N-(Bicyclo[1.1.1]pentan-1-yl)-2-(2,4- difluoro-3-hydroxy-5- (trifluoromethyl)phenyl)benzo[d]oxa- zole-5-carboxamide | 425.0 |
| 1.14 (1) | 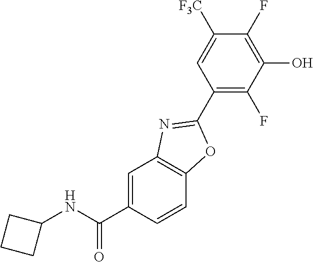 [SMILES: O=C(NC1CCC1)c1ccc2oc(-c3cc(C(F)(F)F)c(F)c(O)c3F)nc2c1] | N-Cyclobutyl-2-(2,4-difluoro-3- hydroxy-5- (trifluoromethyl)phenyl)benzo[d]oxa- zole-5-carboxamide | 413.0 |
| 1.15 (1) | 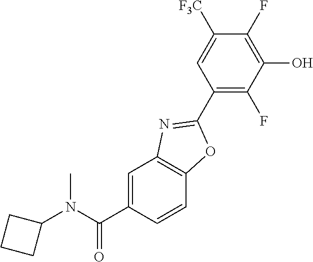 [SMILES: CN(C(=O)c1ccc2oc(-c3cc(C(F)(F)F)c(F)c(O)c3F)nc2c1)C1CCC1] | N-Cyclobutyl-2-(2,4-difluoro-3- hydroxy-5-(trifluoromethyl)phenyl)- N-methylbenzo[d]oxazole-5- carboxamide | 427.1 |
| 1.16 (1) | 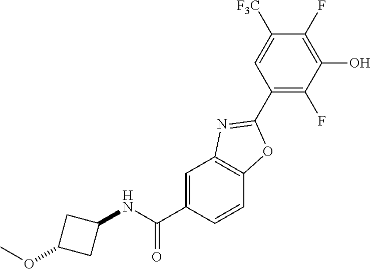 [SMILES: CO[C@H]1C[C@H](NC(=O)c2ccc3oc(-c4cc(C(F)(F)F)c(F)c(O)c4F)nc3c2)C1] | 2-(2,4-Difluoro-3-hydroxy-5- (trifluoromethyl)phenyl)-N-(trans-3- methoxycyclobutyl)benzo[d]oxazole- 5-carboxamide | 443.0 |
| 1.17 (1) | 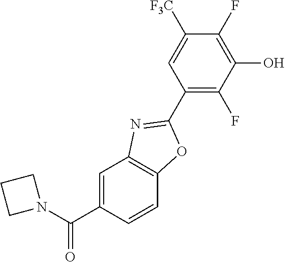 [SMILES: O=C(c1ccc2oc(-c3cc(C(F)(F)F)c(F)c(O)c3F)nc2c1)N1CCC1] | Azetidin-1-yl(2-(2,4-difluoro-3- hydroxy-5- (trifluoromethyl)phenyl)benzo[d]oxa- zol-5-yl)methanone | 398.9 |
| 1.18 (1) | 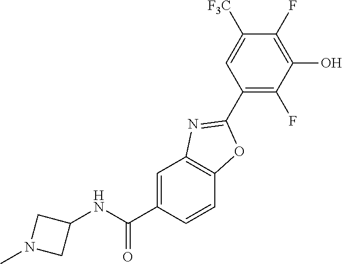 [SMILES: CN1CC(NC(=O)c2ccc3oc(-c4cc(C(F)(F)F)c(F)c(O)c4F)nc3c2)C1] | 2-(2,4-Difluoro-3-hydroxy-5- (trifluoromethyl)phenyl)-N-(1- methylazetidin-3-yl)benzo[d]oxazole- 5-carboxamide | 428.1 |
| 1.19 (1) | 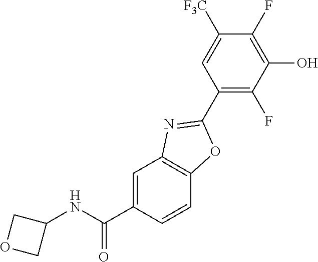 [SMILES: O=C(NC1COC1)c1ccc2oc(-c3cc(C(F)(F)F)c(F)c(O)c3F)nc2c1] | 2-(2,4-Difluoro-3-hydroxy-5- (trifluoromethyl)phenyl)-N-(oxetan- 3-yl)benzo[d]oxazole-5-carboxamide | 415.0 |
| 1.20 (1) | 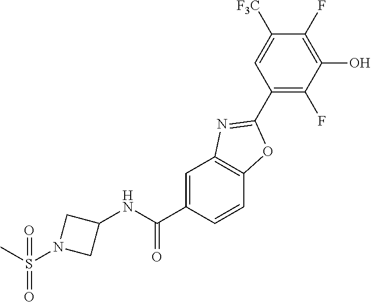 [SMILES: CS(=O)(=O)N1CC(NC(=O)c2ccc3oc(-c4cc(C(F)(F)F)c(F)c(O)c4F)nc3c2)C1] | 2-(2,4-Difluoro-3-hydroxy-5- (trifluoromethyl)phenyl)-N-(1- (methylsulfonyl)azetidin-3- yl)benzo[d]oxazole-5-carboxamide | 492.0 |
| 1.21 (1) | 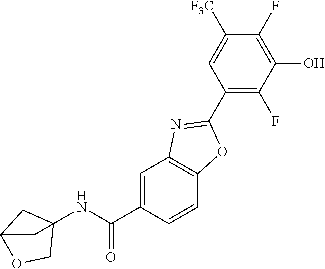 [SMILES: O=C(NC12COC(C1)C2)c1ccc2oc(-c3cc(C(F)(F)F)c(F)c(O)c3F)nc2c1] | N-(2-Oxabicyclo[2.1.1]hexan-4-yl)- 2-(2,4-difluoro-3-hydroxy-5- (trifluoromethyl)phenyl)benzo[d]oxa- zole-5-carboxamide | 441.0 |
| 1.22 (1) | 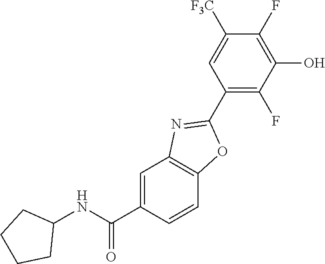 [SMILES: O=C(NC1CCCC1)c1ccc2oc(-c3cc(C(F)(F)F)c(F)c(O)c3F)nc2c1] | N-Cyclopentyl-2-(2,4-difluoro-3- hydroxy-5- (trifluoromethyl)phenyl)benzo[d]oxa- zole-5-carboxamide | 427.1 |
| 1.23 (1) | 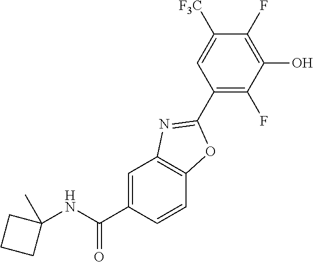 [SMILES: CC1(NC(=O)c2ccc3oc(-c4cc(C(F)(F)F)c(F)c(O)c4F)nc3c2)CCC1] | 2-(2,4-Difluoro-3-hydroxy-5- (trifluoromethyl)phenyl)-N-(1- methylcyclobutyl)benzo[d]oxazole-5- carboxamide | 427.0 |
| 1.24 (2,3) | 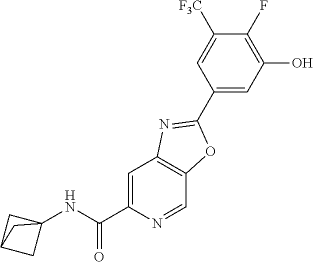 [SMILES: O=C(NC12CC(C1)C2)c1cc2nc(-c3cc(O)c(F)c(C(F)(F)F)c3)oc2cn1] | N-(Bicyclo[1.1.1]pentan-1-yl)-2-(4- fluoro-3-hydroxy-5- (trifluoromethyl)phenyl)oxazolo[5,4- c]pyridine-6-carboxamide | 408.0 |
| 1.25 (1) | 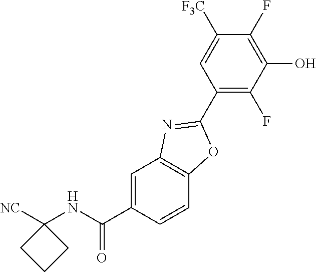 [SMILES: N#CC1(NC(=O)c2ccc3oc(-c4cc(C(F)(F)F)c(F)c(O)c4F)nc3c2)CCC1] | N-(1-Cyanocyclobutyl)-2-(2,4- difluoro-3-hydroxy-5- (trifluoromethyl)phenyl)benzo[d]oxa- zole-5-carboxamide | 438.0 |
| 1.26 (1) | 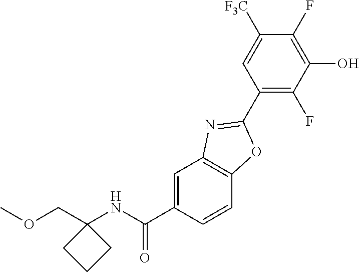 [SMILES: COCC1(NC(=O)c2ccc3oc(-c4cc(C(F)(F)F)c(F)c(O)c4F)nc3c2)CCC1] | 2-(2,4-Difluoro-3-hydroxy-5- (trifluoromethyl)phenyl)-N-(1- (methoxymethyl)cyclobutyl)benzo[d] oxazole-5-carboxamide | 457.0 |
| 1.27 (1) | 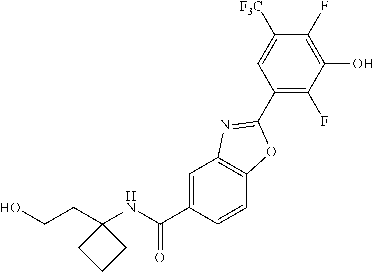 [SMILES: O=C(NC1(CCO)CCC1)c1ccc2oc(-c3cc(C(F)(F)F)c(F)c(O)c3F)nc2c1] | 2-(2,4-Difluoro-3-hydroxy-5- (trifluoromethyl)phenyl)-N-(1-(2- hydroxyethyl)cyclobutyl)benzo[d]oxa- zole-5-carboxamide | 457.0 |
| 1.28 (1) | 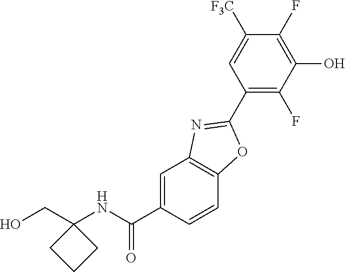 [SMILES: O=C(NC1(CO)CCC1)c1ccc2oc(-c3cc(C(F)(F)F)c(F)c(O)c3F)nc2c1] | 2-(2,4-Difluoro-3-hydroxy-5- (trifluoromethyl)phenyl)-N-(1- (hydroxymethyl)cyclobutyl)benzo[d] oxazole-5-carboxamide | 443.0 |
| 1.29 (1) | 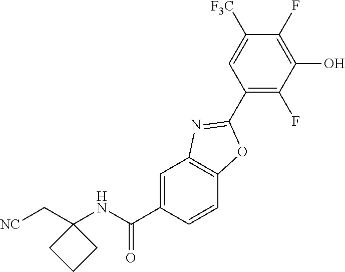 [SMILES: N#CCC1(NC(=O)c2ccc3oc(-c4cc(C(F)(F)F)c(F)c(O)c4F)nc3c2)CCC1] | N-(1-(Cyanomethyl)cyclobutyl)-2- (2,4-difluoro-3-hydroxy-5- (trifluoromethyl)phenyl)benzo[d]oxa- zole-5-carboxamide | 452.0 |
| 1.30 (1) | 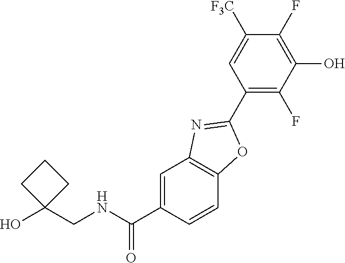 [SMILES: O=C(NCC1(O)CCC1)c1ccc2oc(-c3cc(C(F)(F)F)c(F)c(O)c3F)nc2c1] | 2-(2,4-Difluoro-3-hydroxy-5- (trifluoromethyl)phenyl)-N-((1- hydroxycyclobutyl)methyl)benzo[d] oxazole-5-carboxamide | 443.0 |
| 1.31 (1) | 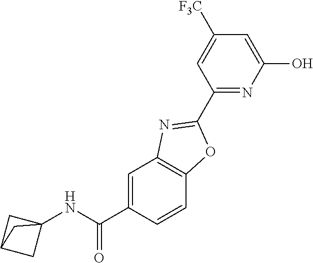 [SMILES: O=C(NC12CC(C1)C2)c1ccc2oc(-c3cc(C(F)(F)F)cc(O)n3)nc2c1] | N-(Bicyclo[1.1.1]pentan-1-yl)-2-(6- hydroxy-4-(trifluoromethyl)pyridin- 2-yl)benzo[d]oxazole-5-carboxamide | 390.0 |

Alternate conditions used:
1. No phenol protecting group (hydrogenation step omitted);
2. MOM protected phenol (deprotected conditions: TFA:DCM, rt);
3. Step 1: amide synthesized from Intermediate 5 and the corresponding amine (AlMe₃ in DCM, rt).

### Compound 2

### N-Cyclobutyl-2-(3-hydroxy-5-(trifluoromethyl)phenyl)benzo[d]oxazole-5-carboxamide

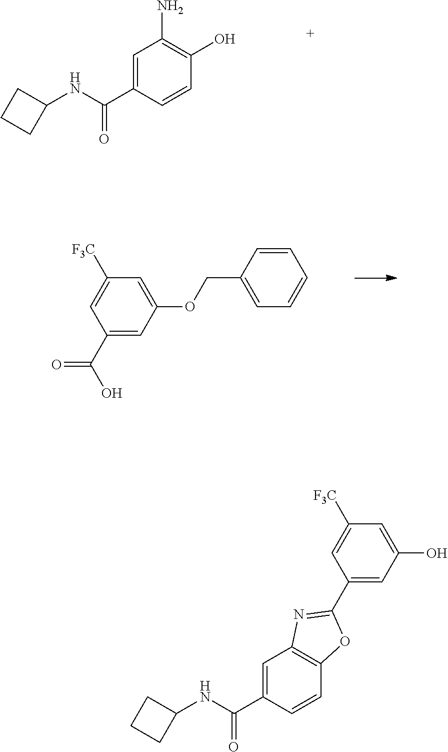 [SMILES: Nc1cc(C(=O)NC2CCC2)ccc1O ; O=C(O)c1cc(OCc2ccccc2)cc(C(F)(F)F)c1 ; O=C(NC1CCC1)c1ccc2oc(-c3cc(O)cc(C(F)(F)F)c3)nc2c1]

DMF (0.1 mL) was added to a solution of Intermediate 10 (300 mg, 1.01 mmol), oxalyl chloride (0.15 mL, 1.75 mmol) and DCM (10 mL) at rt. The mixture was stirred for 100 min, concentrated, dissolved (20 mL DCM), re-concentrated, dried under vacuum for 10 min, and then dissolved in dioxane (5 mL). The solution was added to a suspension of Intermediate 2 (188 mg, 0.91 mmol) in dioxane (10 mL). The reaction was stirred for 10 min. Methanesulfonic acid (0.33 mL, 5.06 mmol) was added. The reaction was heated at 100° C. for 20 h, diluted (100 mL EtOAc), washed (75 mL water and then 75 mL brine), dried (Na₂SO₄), and then concentrated. The residue was purified by silica gel chromatography (0-30% EtOAc/hexanes) to give a white solid (208 mg). The solid was taken up in THF (12 mL). Pd/C (10%, 24 mg) was added. The reaction was stirred under a balloon of hydrogen for 45 min and then filtered. The filter cake was washed with THF (10 mL), and the filtrate was concentrated. The resulting solid was triturated with acetonitrile (5 mL) to give N-cyclobutyl-2-(3-hydroxy-5-(trifluoromethyl)phenyl)benzo[d]oxazole-5-carboxamide (140 mg, 41%) as a white solid. ¹H NMR (400 MHz, DMSO-d₆): δ 10.78 (s, 1H), 8.75 (d, J=7.5 Hz, 1H), 8.34-8.32 (m, 1H), 8.01-7.96 (m, 1H), 7.92-7.85 (m, 3H), 7.33 (s, 1H), 4.52-4.38 (m, 1H), 2.31-2.20 (m, 2H), 2.19-2.01 (m, 2H), 1.78-1.66 (m, 2H); LCMS 377.0 [M+H]⁺.

The Compounds below were synthesized in a similar manner to that described for Compound 2.

| Cmpd | Structure | Name | [M + H]⁺ |
|---|---|---|---|
| 2.01 | 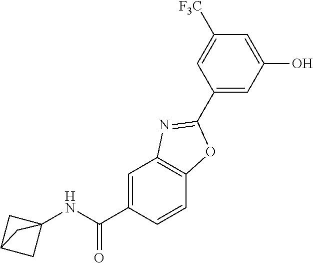 [SMILES: O=C(NC12CC(C1)C2)c1ccc2oc(-c3cc(O)cc(C(F)(F)F)c3)nc2c1] | N-(Bicyclo[1.1.1]pentan-1-yl)-2-(3- hydroxy-5- (trifluoromethyl)phenyl)benzo[d]oxa- zole-5-carboxamide | 389.0 |
| 2.02 (1) | 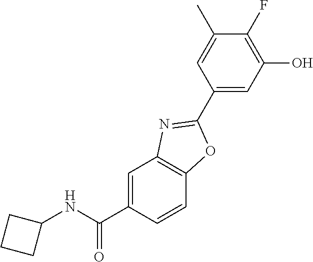 [SMILES: Cc1cc(-c2nc3cc(C(=O)NC4CCC4)ccc3o2)cc(O)c1F] | N-Cyclobutyl-2-(4-fluoro-3-hydroxy- 5-methylphenyl)benzo[d]oxazole-5- carboxamide | 341.0 |

### Compound 3

### 2-Chloro-4-(2-(4-fluoro-3-hydroxyphenyl)benzo[d]oxazol-5-yl)phenol

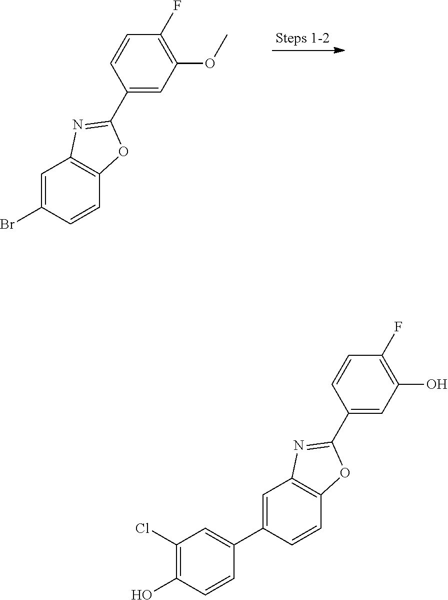 [SMILES: COc1cc(-c2nc3cc(Br)ccc3o2)ccc1F ; Oc1cc(-c2nc3cc(-c4ccc(O)c(Cl)c4)ccc3o2)ccc1F]

### Step 1: 5-(3-Chloro-4-methoxyphenyl)-2-(4-fluoro-3-methoxyphenyl)benzo[d]oxazole

A mixture of Intermediate 1.01 (0.12 g, 0.37 mmol), 3-chloro-4-methoxyphenylboronic acid (0.11 g, 0.57 mmol), Pd(PPh₃)₄ (0.05 g, 0.04 mmol), Na₂CO₃ (2 M, 0.4 mL, 0.8 mmol), and dioxane (2 mL) was heated at 80° C. for 60 min, allowed to cool to rt, diluted (20 mL EtOAc), washed (20 mL water and then 20 mL brine), dried (Na₂SO₄), and then concentrated. The residue was purified by silica gel chromatography (0-15% EtOAc in hexanes) to give 5-(3-chloro-4-methoxyphenyl)-2-(4-fluoro-3-methoxyphenyl)benzo[d]oxazole (80 mg, 58%) as a beige solid. ¹H NMR (400 MHz, DMSO-d₆): δ 8.06 (d, J=1.7 Hz, 1H), 7.91 (dd, J=2.0, 8.2 Hz, 1H), 7.88-7.79 (m, 3H), 7.71 (ddd, J=2.1, 3.9, 8.6 Hz, 2H), 7.56-7.43 (m, 1H), 7.27 (d, J=8.7 Hz, 1H), 4.01 (s, 3H), 3.92 (s, 3H); LCMS: 384.0 [M+H]⁺.

### Step 2: 2-Chloro-4-(2-(4-fluoro-3-hydroxyphenyl)benzo[d]oxazol-5-yl)phenol

A solution of 5-(3-chloro-4-methoxyphenyl)-2-(4-fluoro-3-methoxyphenyl)benzo[d]oxazole (0.08 g, 0.21 mmol) in DCM (3 mL) was cooled in an ice/water bath. Boron tribromide (1 M in DCM, 1.1 mL, 1.1 mmol) was added. The mixture was stirred at rt overnight, cooled in an ice bath, quenched (3 mL methanol), allowed to warm to rt, and then concentrated. The residue was purified by prep-HPLC to give 2-chloro-4-(2-(4-fluoro-3-hydroxyphenyl)benzo[d]oxazol-5-yl)phenol (52 mg, 70%) as a yellow solid. ¹H NMR (400 MHz, DMSO-d₆): δ 10.53 (s, 1H), 10.34 (s, 1H), 8.01 (d, J=1.6 Hz, 1H), 7.83-7.79 (m, 2H), 7.72 (d, J=2.3 Hz, 1H), 7.70-7.63 (m, 2H), 7.54 (dd, J=2.3, 8.4 Hz, 1H), 7.40 (dd, J=8.6, 11.0 Hz, 1H), 7.08 (d, J=8.4 Hz, 1H); LCMS: 355.9 [M+H]⁺.

The Compounds below were synthesized in a similar manner to that described for Compound 3.

| Cmpd | Structure | Name | [M + H]⁺ |
|---|---|---|---|
| 3.01 | 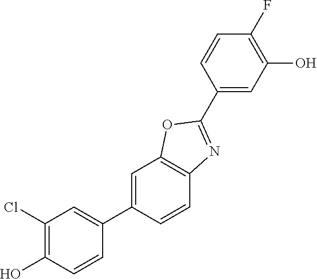 [SMILES: Oc1cc(-c2nc3ccc(-c4ccc(O)c(Cl)c4)cc3o2)ccc1F] | 2-Chloro-4-(2-(4-fluoro-3- hydroxyphenyl)benzo[d]oxazol-6- yl)phenol | 355.9 |
| 3.02 (1) | 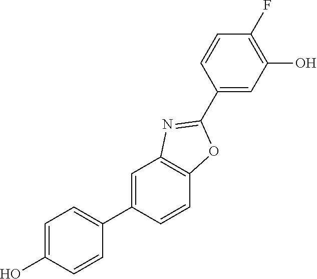 [SMILES: Oc1ccc(-c2ccc3oc(-c4ccc(F)c(O)c4)nc3c2)cc1] | 2-Fluoro-5-(5-(4- hydroxyphenyl)benzo[d]oxazol-2- yl)phenol | 322.0 |
| 3.03 (1) | 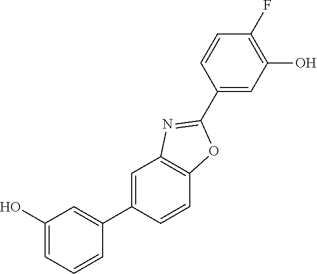 [SMILES: Oc1cccc(-c2ccc3oc(-c4ccc(F)c(O)c4)nc3c2)c1] | 2-Fluoro-5-(5-(3- hydroxyphenyl)benzo[d]oxazol-2- yl)phenol | 322.1 |

Alternate conditions used:
1. Boronic acid of unprotected phenol was used.

### Compound 4

### 2-Fluoro-5-(5-(4-(methylsulfonyl)piperazin-1-yl)benzo[d]oxazol-2-yl)phenol

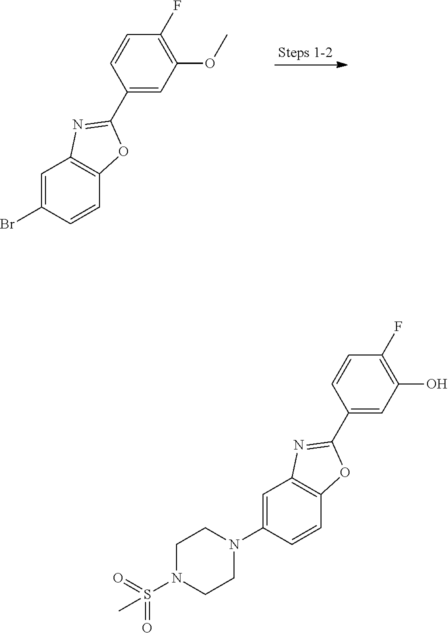 [SMILES: COc1cc(-c2nc3cc(Br)ccc3o2)ccc1F ; CS(=O)(=O)N1CCN(c2ccc3oc(-c4ccc(F)c(O)c4)nc3c2)CC1]

### Step 1: 2-(4-Fluoro-3-methoxyphenyl)-5-(4-(methylsulfonyl)piperazin-1-yl)benzo[d]oxazole

A mixture of Intermediate 1.01 (103 mg, 0.32 mmol), 1-methanesulfonyl-piperazine (79 mg, 0.48 mmol), RuPhos (9 mg, 0.02 mmol), NaO^{t}Bu (65 mg, 0.68 mmol), Pd₂(dba)₃ (60 mg, 0.07 mmol), and toluene (4 mL) was heated at 100° C. overnight, allowed to cool to rt, diluted (20 mL EtOAc), washed (15 mL water, and then 15 mL brine), dried (Na₂SO₄), and then concentrated. The residue was purified by silica gel chromatography (20-60% EtOAc in hexanes) to give 2-(4-fluoro-3-methoxyphenyl)-5-(4-(methylsulfonyl)piperazin-1-yl)benzo[d]oxazole (65 mg, 50%) as a tan solid. ¹H NMR (400 MHz, DMSO-d₆): δ 7.86 (dd, J=2.0, 8.3 Hz, 1H), 7.77 (ddd, J=2.0, 4.4, 8.5 Hz, 1H), 7.67 (d, J=9.0 Hz, 1H), 7.47 (dd, J=8.5, 11.2 Hz, 1H), 7.36 (d, J=2.3 Hz, 1H), 7.16 (dd, J=2.4, 9.0 Hz, 1H), 3.99 (s, 3H), 3.29 (s, 8H), 2.95 (s, 3H); LCMS: 406.1 [M+H]⁺.

### Step 2: 2-Fluoro-5-(5-(4-(methylsulfonyl)piperazin-1-yl)benzo[d]oxazol-2-yl)phenol

A solution of 2-(4-fluoro-3-methoxyphenyl)-5-(4-(methylsulfonyl)piperazin-1-yl)benzo[d]oxazole (65 mg, 0.16 mmol) and DCM (3 mL) was cooled in an ice/water bath. Boron tribromide (1 M in DCM, 1.2 mL, 1.2 mmol) was added. After stirring for 5 min, the ice bath was removed. The mixture was stirred at rt overnight, cooled in an ice bath, quenched (4 mL methanol), allowed to warm to rt, and then concentrated. The residue was purified by prep-HPLC to give 2-fluoro-5-(5-(4-(methylsulfonyl)piperazin-1-yl)benzo[d]oxazol-2-yl)phenol (13 mg, 21%) as a pale yellow solid. ¹H NMR (400 MHz, DMSO-d₆): δ 10.49 (s, 1H), 7.76 (dd, J=2.1, 8.4 Hz, 1H), 7.67-7.59 (m, 2H), 7.40-7.33 (m, 2H), 7.14 (dd, J=2.4, 9.0 Hz, 1H), 3.28 (s, 8H), 2.95 (s, 3H); LCMS: 392.0 [M+H]⁺.

The Compounds below were synthesized in a similar manner to that described for Compound 4.

| Cmpd | Structure | Name | [M + H]⁺ |
|---|---|---|---|
| 4.01 | 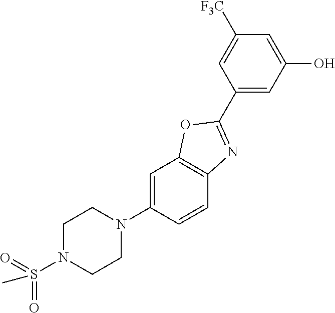 [SMILES: CS(=O)(=O)N1CCN(c2ccc3nc(-c4cc(O)cc(C(F)(F)F)c4)oc3c2)CC1] | 3-(6-(4-(Methylsulfonyl)piperazin-1- yl)benzo[d]oxazol-2-yl)-5- (trifluoromethyl)phenol | 442.0 |
| 4.02 | 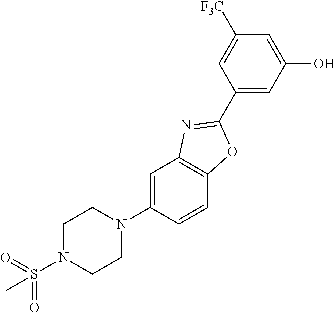 [SMILES: CS(=O)(=O)N1CCN(c2ccc3oc(-c4cc(O)cc(C(F)(F)F)c4)nc3c2)CC1] | 3-(5-(4-(Methylsulfonyl)piperazin-1- yl)benzo[d]oxazol-2-yl)-5- (trifluoromethyl)phenol | 442.0 |

### Compound 5

### 5-(6-Chloro-5-(4-(methylsulfonyl)piperazin-1-yl)benzo[d]oxazol-2-yl)-2,3-difluorophenol

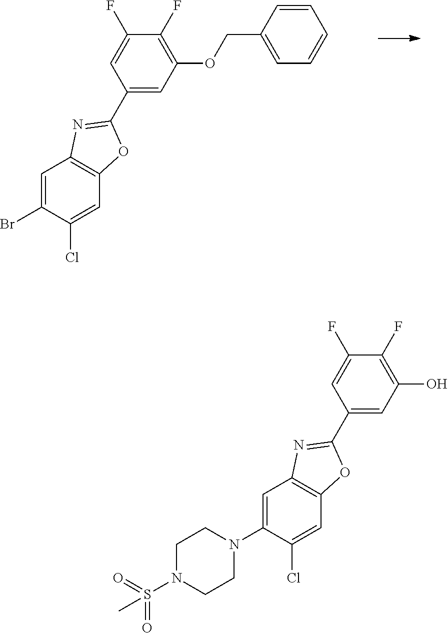 [SMILES: Fc1cc(-c2nc3cc(Br)c(Cl)cc3o2)cc(OCc2ccccc2)c1F ; CS(=O)(=O)N1CCN(c2cc3nc(-c4cc(O)c(F)c(F)c4)oc3cc2Cl)CC1]

A mixture of Intermediate 1.03 (105 mg, 0.23 mmol), 1-methanesulfonyl-piperazine (73 mg, 0.36 mmol), RuPhos (6 mg, 0.01 mmol), NaO^{t}Bu (47 mg, 0.49 mmol), Pd₂(dba)₃ (45 mg, 0.05 mmol), and toluene (3 mL) was heated at 100° C. for 150 min, allowed to cool to rt, diluted (20 mL EtOAc), washed (20 mL water and then 20 mL brine), dried (Na₂SO₄), and then concentrated. The residue was purified by silica gel chromatography (0-30% EtOAc in hexanes). The intermediate product was dissolved in THF (10 mL). Palladium on carbon (10%, 20 mg) was added. The mixture stirred under a balloon of hydrogen for 30 min and then filtered. The filter cake was rinsed (10 ml THF), and the filtrate was concentrated. The residue was purified by prep-HPLC to give 5-(6-chloro-5-(4-(methylsulfonyl)piperazin-1-yl)benzo[d]oxazol-2-yl)-2,3-difluorophenol (5 mg, 5%) as a white solid. ¹H NMR (400 MHz, DMSO-d₆): δ 11.13 (s, 1H), 8.04 (s, 1H), 7.70 (s, 1H), 7.63-7.54 (m, 2H), 3.31 (d, J=4.6 Hz, 4H), 3.15-3.07 (m, 4H), 2.97 (s, 3H); LCMS: 443.9 [M+H]⁺.

The Compounds below were synthesized in a similar manner to that described for Compound 5.

| Cmpd | Structure | Name | [M + H]⁺ |
|---|---|---|---|
| 5.01 (1) | 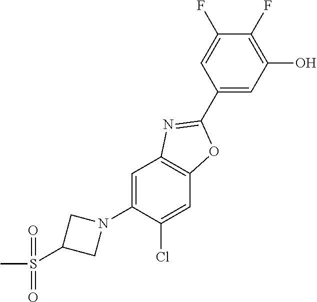 [SMILES: CS(=O)(=O)C1CN(c2cc3nc(-c4cc(O)c(F)c(F)c4)oc3cc2Cl)C1] | 5-(6-Chloro-5-(3- (methylsulfonyl)azetidine-1- yl)benzo[d]oxazol-2-yl)-2,3- difluorophenol | 414.9 |
| 5.02 | 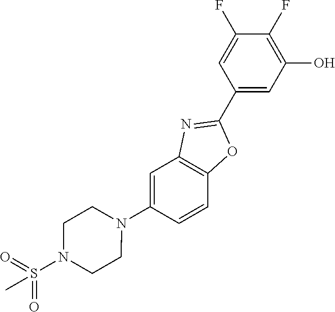 [SMILES: CS(=O)(=O)N1CCN(c2ccc3oc(-c4cc(O)c(F)c(F)c4)nc3c2)CC1] | 2,3-Difluoro-5-(5-(4- (methylsulfonyl)piperazin-1- yl)benzo[d]oxazol-2-yl)phenol | 410.1 |

Alternate condition used:
1. Pd(OAc)₂, P(^{t}Bu)₃, NaO^{t}Bu, PhMe, 100° C.

### Compound 6

### 2-Fluoro-5-(5-(4-(methylsulfonyl)piperazin-1-yl)benzo[d]oxazol-2-yl)-3-(trifluoromethyl)phenol

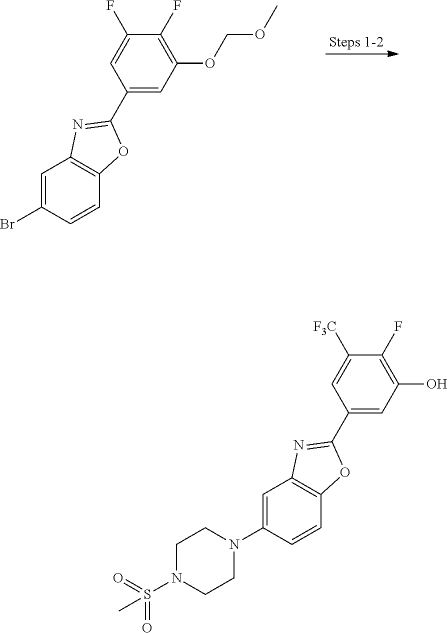 [SMILES: COCOc1cc(-c2nc3cc(Br)ccc3o2)cc(F)c1F ; CS(=O)(=O)N1CCN(c2ccc3oc(-c4cc(O)c(F)c(C(F)(F)F)c4)nc3c2)CC1]

### Step 1: 2-(4-Fluoro-3-(methoxymethoxy)-5-(trifluoromethyl)phenyl)-5-(4-(methylsulfonyl)piperazin-1-yl)benzo[d]oxazole

Palladium(II) acetate (5.3 mg, 0.024 mmol) was added to a mixture of Intermediate 8.02 (100 mg, 0.24 mmol), 1-methylsufonylpiperazine (117 mg, 0.71 mmol), NaO^{t}Bu (183 mg, 1.90 mmol), P^{t}Bu₃ (560 μL, 0.24 mmol, 10% in n-hexane), and toluene (5 mL). The mixture was degassed with 3 vacuum/N₂ cycles, heated at 80° C. for 1 h, allowed to cool to rt, poured into H₂O (30 mL), and then extracted (3×30 mL EtOAc). The combined organic layers were washed (50 mL brine), dried (Na₂SO₄), filtered, and then concentrated. The residue was purified by silica gel chromatography (50% EtOAc/petroleum ether) to give 2-(4-fluoro-3-(methoxymethoxy)-5-(trifluoromethyl)phenyl)-5-(4-(methylsulfonyl)piperazin-1-yl)benzo[d]oxazole (60 mg, 50%) as a yellow solid. LCMS: 504.0 [M+H]⁺.

### Step 2: 2-Fluoro-5-(5-(4-(methylsulfonyl)piperazin-1-yl)benzo[d]oxazol-2-yl)-3-(trifluoromethyl)phenol

A mixture of 2-(4-fluoro-3-(methoxymethoxy)-5-(trifluoromethyl)phenyl)-5-(4-(methylsulfonyl)piperazin-1-yl)benzo[d]oxazole (55 mg, 0.11 mmol), TFA (500 μL, 6.55 mmol), and DCM (20 mL) was stirred at rt for 2 h, poured into saturated NaHCO₃ (20 mL), and then extracted (3×20 mL EtOAc). The organic layers were combined, washed (20 mL brine), dried (Na₂SO₄), filtered, and then concentrated. The residue was purified by prep-HPLC [water (0.04% HCl)/CH₃CN] to give 2-fluoro-5-(5-(4-(methylsulfonyl)piperazin-1-yl)benzo[d]oxazol-2-yl)-3-(trifluoromethyl)phenol (31 mg, 62%) as a white solid. ¹H NMR (400 MHz, DMSO-d₆): δ 11.34 (s, 1H), 8.03 (d, 1H), 7.81 (d, 1H), 7.69 (d, 1H), 7.37 (s, 1H), 7.19 (d, 1H), 3.29 (s, 8H), 2.94 (s, 3H); LCMS: 459.9 [M+H]⁺.

The Compounds below were synthesized in a similar manner to that described for Compound 6.

| Cmpd | Structure | Name | [M + H]⁺ |
|---|---|---|---|
| 6.01 (1) | 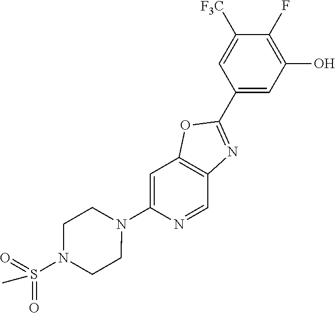 [SMILES: CS(=O)(=O)N1CCN(c2cc3oc(-c4cc(O)c(F)c(C(F)(F)F)c4)nc3cn2)CC1] | 2-Fluoro-5-(6-(4- (methylsulfonyl)piperazin-1- yl)oxazolo[4,5-c]pyridin-2-yl)-3- (trifluoromethyl)phenol | 461.0 |
| 6.02 (1) | 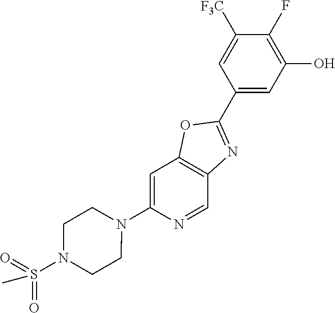 [SMILES: CS(=O)(=O)N1CCN(c2cc3oc(-c4cc(O)c(F)c(C(F)(F)F)c4)nc3cn2)CC1] | 2-Fluoro-5-(6-(4- (methylsulfonyl)piperazin-1- yl)benzo[d]oxazol-2-yl)-3- (trifluoromethyl)phenol | 460.0 |

Alternate conditions used:
1. Step 1: Pd₂(dba)₃, BINAP, Cs₂CO₃ or NaOtBu, toluene, 100° C., 3 h-ON.

### Compound 7

### 2,6-Difluoro-3-(5-(4-(methylsulfonyl)piperazin-1-yl)benzo[d]oxazol-2-yl)-5-(trifluoromethyl)phenol

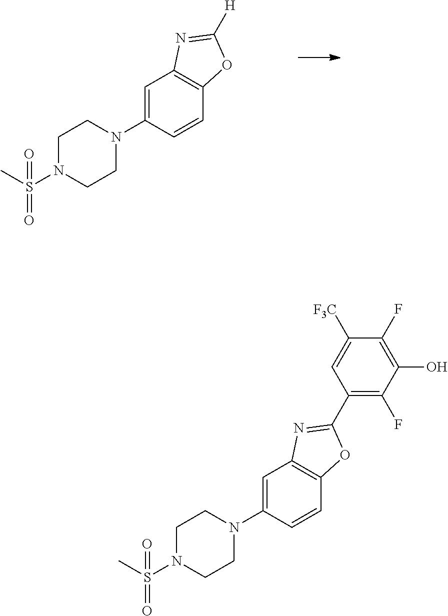 [SMILES: CS(=O)(=O)N1CCN(c2ccc3ocnc3c2)CC1 ; CS(=O)(=O)N1CCN(c2ccc3oc(-c4cc(C(F)(F)F)c(F)c(O)c4F)nc3c2)CC1]

A mixture of Intermediate 7 (42 mg, 0.15 mmol), Intermediate 11, Step 2 (23 mg, 0.08 mmol), Pd(OAc)₂ (5 mg, 0.02 mmol), Cu(OAc)₂ (7 mg, 0.04 mmol), K₂CO₃ (43 mg, 0.31 mmol), and toluene (1 mL) was heated at 160° C. in a microwave for 30 min then diluted (20 ml EtOAc and 20 mL saturated NH₄Cl). The resulting solid was collected by filtration and purified by prep-HPLC to give 2,6-difluoro-3-(5-(4-(methylsulfonyl)piperazin-1-yl)benzo[d]oxazol-2-yl)-5-(trifluoromethyl)phenol (10 mg, 14%) as a white powder. ¹H NMR (400 MHz, DMSO-d₆): δ 11.97-11.42 (m, 1H), 7.88 (br t, J=6.3 Hz, 1H), 7.72 (br d, J=9.0 Hz, 1H), 7.41 (br s, 1H), 7.23 (br d, J=8.7 Hz, 1H), 3.29 (br s, 8H), 2.95 (s, 3H); LCMS 478.0 [M+H]⁺.

The Compounds below were synthesized in a similar manner to that described for Compound 7.

| Cmpd | Structure | Name | [M + H]⁺ |
|---|---|---|---|
| 7.01 (1) | 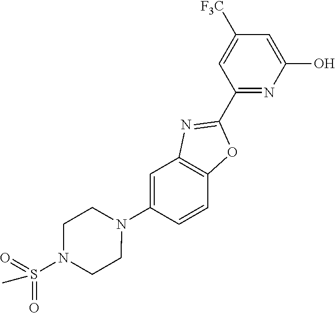 [SMILES: CS(=O)(=O)N1CCN(c2ccc3oc(-c4cc(C(F)(F)F)cc(O)n4)nc3c2)CC1] | 6-(5-(4-(Methylsulfonyl)piperazin-1- yl)benzo[d]oxazol-2-yl)-4- (trifluoromethyl)pyridin-2-ol | 443.0 |
| 7.02 (1) | 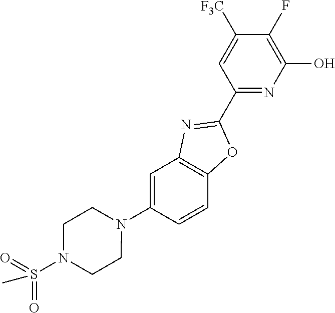 [SMILES: CS(=O)(=O)N1CCN(c2ccc3oc(-c4cc(C(F)(F)F)c(F)c(O)n4)nc3c2)CC1] | 3-Fluoro-6-(5-(4- (methylsulfonyl)piperazin-1- yl)benzo[d]oxazol-2-yl)-4- (trifluoromethyl)pyridin-2-ol | 461.0 |

Alternate conditions used: Reaction time was 30-45 min.
1. PCy₃ was also used.

### Compound 8

### 2,6-Difluoro-3-(1-methyl-6-(7-oxa-4-azaspiro[2.5]octan-4-yl)-1H-pyrazolo[4,3-c]pyridin-3-yl)-5-trifluoromethyl phenol

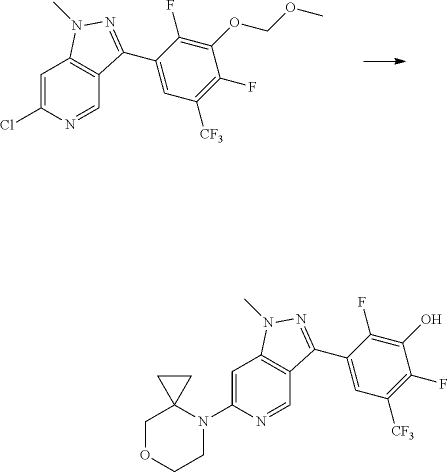 [SMILES: COCOc1c(F)c(-c2nn(C)c3cc(Cl)ncc23)cc(C(F)(F)F)c1F ; Cn1nc(-c2cc(C(F)(F)F)c(F)c(O)c2F)c2cnc(N3CCOCC34CC4)cc21]

A mixture of Intermediate 18 (50 mg, 0.12 mmol), 5-oxa-8-azaspiro[2.5]octane (21 mg, 0.18 mmol), Pd₂(dba)₃ (5.6 mg, 0.0061 mmol), RuPhos (5.7 mg, 0.012 mmol), and NaO^{t}Bu (47 mg, 0.49 mmol) in dioxane (2 mL) was purged with nitrogen for 5 min, heated in a microwave at 90° C. for 90 min, partitioned between DCM and saturated ammonium chloride, and then filtered through a pad of Celite. The aqueous layer was separated and extracted with DCM. The combined organics were washed with saturated ammonium chloride, dried (Na₂SO₄), and then concentrated under reduced pressure. The residue was taken up in DCM (2.0 mL) and TFA (2.0 mL), stirred at rt for 1 h, and then concentrated under reduced pressure. The residue was purified by prep-HPLC (5-95% CH₃CN:H₂O) to give 2,6-difluoro-3-[1-methyl-6-(7-oxa-4-azaspiro[2.5]oct-4-yl)pyrazolo[4,5-c]pyridin-3-yl]-5-(trifluoromethyl)phenol (28 mg, 52%) as a white powder. ¹H NMR (400 MHz, DMSO-d₆): δ 11.65-11.12 (m, 1H), 8.78 (d, J=2.1 Hz, 1H), 7.52 (t, J=7.0 Hz, 1H), 7.00 (s, 1H), 4.05 (s, 5H), 3.76-3.56 (m, 2H), 3.53 (br s, 2H), 1.08 (br s, 2H), 0.93 (br s, 2H); LCMS: 441.0 [M+H]⁺.

### Compound 9

### 3-(6-(Cyclopropyl(methyl)amino)-1-methyl-1H-pyrazolo[4,3-c]pyridin-3-yl)-2,6-difluoro-5-(trifluoromethyl)phenol

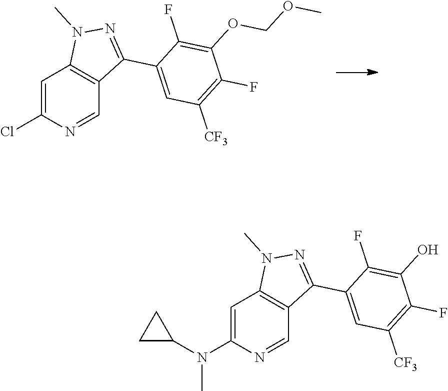 [SMILES: COCOc1c(F)c(-c2nn(C)c3cc(Cl)ncc23)cc(C(F)(F)F)c1F ; CN(c1cc2c(cn1)c(-c1cc(C(F)(F)F)c(F)c(O)c1F)nn2C)C1CC1]

A mixture of Intermediate 18 (50 mg, 0.12 mmol), N-methylcyclopropanamine (13 mg, 0.18 mmol), NaO^{t}Bu (47 mg, 0.49 mmol), RuPhos (5.7 mg, 0.01 mmol), and Pd₂(dba)₃ (5.6 mg, 0.01 mmol) in dioxane (2 mL) was purged with nitrogen for 5 min, heated in a microwave at 90° C. for 90 min, and then partitioned between DCM and saturated ammonium chloride. The aqueous layer was separated and extracted with DCM. The combined organics were washed with saturated ammonium chloride, dried (Na₂SO₄), and then concentrated under reduced pressure. The residue was taken up in DCM (2 mL) and TFA (2 mL), stirred at rt for 30 min, and then concentrated under reduced pressure. The residue was purified by prep-HPLC (5-95% CH₃CN:H₂O) to give 3-[6-(cyclopropylmethylamino)-1-methylpyrazolo[4,5-c]pyridin-3-yl]-2,6-difluoro-5-(trifluoromethyl)phenol (32 mg, 66%) as a white powder. ¹H NMR (400 MHz, DMSO-d₆): δ 11.80-11.10 (m, 1H), 8.77 (br s, 1H), 7.54 (t, J=7.0 Hz, 1H), 6.97 (s, 1H), 4.05 (s, 3H), 3.19 (s, 3H), 2.66 (br s, 1H), 1.00 (br d, J=5.9 Hz, 2H), 0.69 (br s, 2H); LCMS 399.2 [M+H]⁺.

The Compounds below were synthesized in a similar manner to that described for Compound 9.

| Cmpd | Structure | Name | [M + H]⁺ |
|---|---|---|---|
| 9.01 | 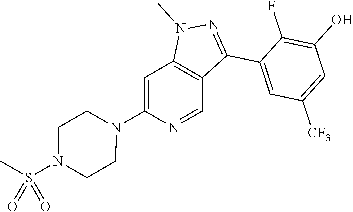 [SMILES: Cn1nc(-c2cc(C(F)(F)F)cc(O)c2F)c2cnc(N3CCN(S(C)(=O)=O)CC3)cc21] | 2-Fluoro-3-(1-methyl-6-(4- (methylsulfonyl)piperazin-1-yl)-1H- pyrazolo[4,3-c]pyridin-3-yl)-5- (trifluoromethyl)phenol | 474.0 |
| 9.02 (1) | 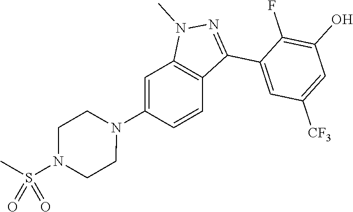 [SMILES: Cn1nc(-c2cc(C(F)(F)F)cc(O)c2F)c2ccc(N3CCN(S(C)(=O)=O)CC3)cc21] | 2-Fluoro-3-(1-methyl-6-(4- (methylsulfonyl)piperazin-1-yl)-1H- indazol-3-yl)-5- (trifluoromethyl)phenol | 473.0 |
| 9.03 | 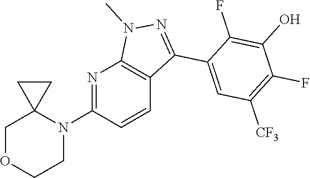 [SMILES: Cn1nc(-c2cc(C(F)(F)F)c(F)c(O)c2F)c2ccc(N3CCOCC34CC4)nc21] | 2,6-Difluoro-3-(1-methyl-6-(7-oxa-4- azaspiro[2.5]octan-4-yl)-1H- pyrazolo[3,4-b]pyridin-3-yl)-5- (trifluoromethyl)phenol | 441.0 |
| 9.04 | 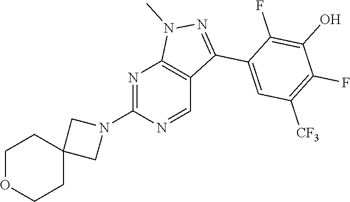 [SMILES: Cn1nc(-c2cc(C(F)(F)F)c(F)c(O)c2F)c2cnc(N3CC4(CCOCC4)C3)nc21] | 2,6-Difluoro-3-(1-methyl-6-(7-oxa-2- azaspiro[3.5]nonan-2-yl)-1H- pyrazolo[3,4-d]pyrimidin-3-yl)-5- (trifluoromethyl)phenol | 456.1 |
| 9.05 | 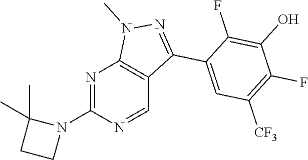 [SMILES: Cn1nc(-c2cc(C(F)(F)F)c(F)c(O)c2F)c2cnc(N3CCC3(C)C)nc21] | 3-(6-(2,2-Dimethylazetidin-1-yl)-1- methyl-1H-pyrazolo[3,4-d]pyrimidin- 3-yl)-2,6-difluoro-5- (trifluoromethyl)phenol | 414.1 |
| 9.06 | 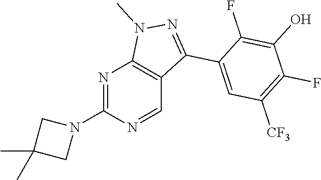 [SMILES: Cn1nc(-c2cc(C(F)(F)F)c(F)c(O)c2F)c2cnc(N3CC(C)(C)C3)nc21] | 3-(6-(3,3-Dimethylazetidin-1-yl)-1- methyl-1H-pyrazolo[3,4-d]pyrimidin- 3-yl)-2,6-difluoro-5- (trifluoromethyl)phenol | 414.3 |
| 9.07 | 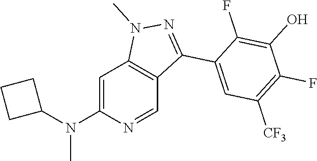 [SMILES: CN(c1cc2c(cn1)c(-c1cc(C(F)(F)F)c(F)c(O)c1F)nn2C)C1CCC1] | 3-(6-(Cyclobutyl(methyl)amino)-1- methyl-1H-pyrazolo[4,3-c]pyridin-3- yl)-2,6-difluoro-5- (trifluoromethyl)phenol | 413.2 |
| 9.08 | 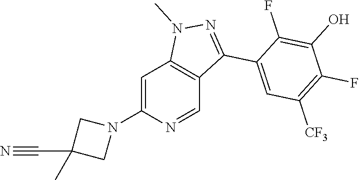 [SMILES: Cn1nc(-c2cc(C(F)(F)F)c(F)c(O)c2F)c2cnc(N3CC(C)(C#N)C3)cc21] | 1-(3-(2,4-Difluoro-3-hydroxy-5- (trifluoromethyl)phenyl)-1-methyl- 1H-pyrazolo[4,3-c]pyridin-6-yl)-3- methylazetidine-3-carbonitrile | 424.0 |
| 9.09 | 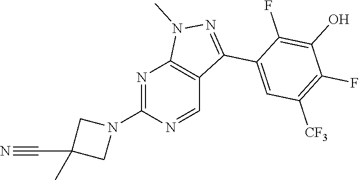 [SMILES: Cn1nc(-c2cc(C(F)(F)F)c(F)c(O)c2F)c2cnc(N3CC(C)(C#N)C3)nc21] | 1-(3-(2,4-Difluoro-3-hydroxy-5- (trifluoromethyl)phenyl)-1-methyl- 1H-pyrazolo[3,4-d]pyrimidin-6-yl)-3- methylazetidine-3-carbonitrile | 425.2 |
| 9.10 | 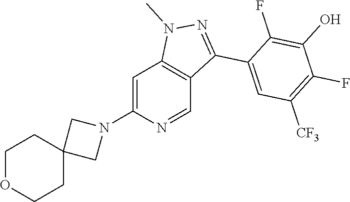 [SMILES: Cn1nc(-c2cc(C(F)(F)F)c(F)c(O)c2F)c2cnc(N3CC4(CCOCC4)C3)cc21] | 2,6-Difluoro-3-(1-methyl-6-(7-oxa-2- azaspiro[3.5]nonan-2-yl)-1H- pyrazolo[4,3-c]pyridin-3-yl)-5- (trifluoromethyl)phenol | 455.2 |
| 9.11 | 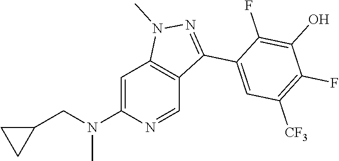 [SMILES: CN(CC1CC1)c1cc2c(cn1)c(-c1cc(C(F)(F)F)c(F)c(O)c1F)nn2C] | 3-(6-((Cyclopropylmethyl) (methyl)amino)-1-methyl-1H- pyrazolo[4,3-c]pyridin-3-yl)-2,6- difluoro-5-(trifluoromethyl)phenol | 413.3 |
| 9.12 (1) | 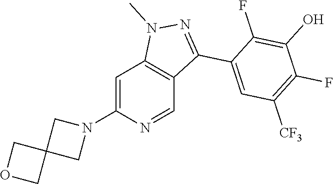 [SMILES: Cn1nc(-c2cc(C(F)(F)F)c(F)c(O)c2F)c2cnc(N3CC4(COC4)C3)cc21] | 2,6-Difluoro-3-(1-methyl-6-(2-oxa-6- azaspiro[3.3]heptan-6-yl)-1H- pyrazolo[4,3-c]pyridin-3-yl)-5- (trifluoromethyl)phenol | 427.1 |
| 9.13 | 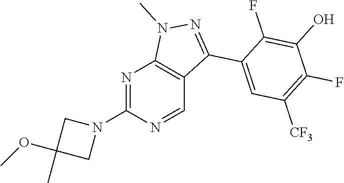 [SMILES: COC1(C)CN(c2ncc3c(-c4cc(C(F)(F)F)c(F)c(O)c4F)nn(C)c3n2)C1] | 2,6-Difluoro-3-(6-(3-methoxy-3- methylazetidin-1-yl)-1-methyl-1H- pyrazolo[3,4-d]pyrimidin-3-yl)-5- (trifluoromethyl)phenol | 430.1 |
| 9.14 | 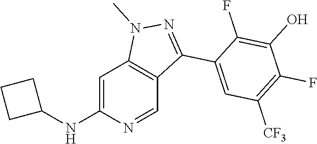 [SMILES: Cn1nc(-c2cc(C(F)(F)F)c(F)c(O)c2F)c2cnc(NC3CCC3)cc21] | 3-(6-(Cyclobutylamino)-1-methyl- 1H-pyrazolo[4,3-c]pyridin-3-yl)-2,6- difluoro-5-(trifluoromethyl)phenol | 399.1 |
| 9.15 | 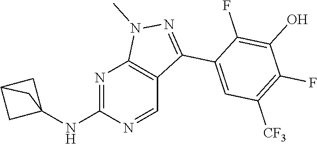 [SMILES: Cn1nc(-c2cc(C(F)(F)F)c(F)c(O)c2F)c2cnc(NC34CC(C3)C4)nc21] | 3-(6-(Bicyclo[1.1.1 ]pentan-1- ylamino)-1-methyl-1H-pyrazolo[3,4- d]pyrimidin-3-yl)-2,6-difluoro-5- (trifluoromethyl)phenol | 412.2 |
| 9.16 | 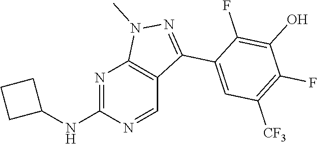 [SMILES: Cn1nc(-c2cc(C(F)(F)F)c(F)c(O)c2F)c2cnc(NC3CCC3)nc21] | 3-(6-(Cyclobutylamino)-1-methyl- 1H-pyrazolo[3,4-d]pyrimidin-3-yl)- 2,6-difluoro-5- (trifluoromethyl)phenol | 400.0 |
| 9.17 | 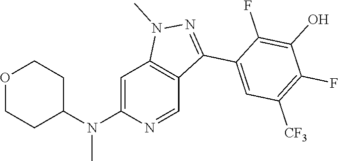 [SMILES: CN(c1cc2c(cn1)c(-c1cc(C(F)(F)F)c(F)c(O)c1F)nn2C)C1CCOCC1] | 2,6-Difluoro-3-(1-methyl-6- (methyl(tetrahydro-2H-pyran-4- yl)amino)-1H-pyrazolo[4,3- c]pyridin-3-yl)-5- (trifluoromethyl)phenol | 443.1 |
| 9.18 | 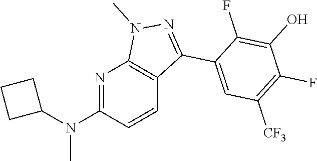 [SMILES: CN(c1ccc2c(-c3cc(C(F)(F)F)c(F)c(O)c3F)nn(C)c2n1)C1CCC1] | 3-(6-(Cyclobutyl(methyl)amino)-1- methyl-1H-pyrazolo[3,4-b]pyridin-3- yl)-2,6-difluoro-5- (trifluoromethyl)phenol | 413.1 |
| 9.19 | 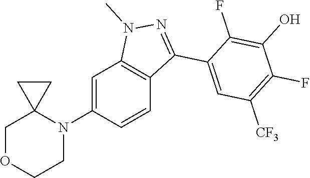 [SMILES: Cn1nc(-c2cc(C(F)(F)F)c(F)c(O)c2F)c2ccc(N3CCOCC34CC4)cc21] | 2,6-Difluoro-3-(1-methyl-6-(7-oxa-4- azaspiro[2.5]octan-4-yl)-1H-indazol- 3-yl)-5-(trifluoromethyl)phenol | 440.3 |
| 9.20 | 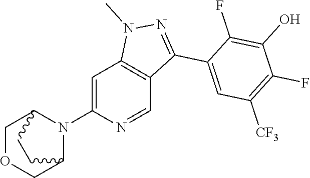 [SMILES: Cn1nc(-c2cc(C(F)(F)F)c(F)c(O)c2F)c2cnc(N3C4CCC3COC4)cc21] | 3-(6-(3-Oxa-8-azabicyclo [3.2.1]octan-8-yl)-1-methyl-1H- pyrazolo[4,3-c]pyridin-3-yl)-2,6- difluoro-5-(trifluoromethyl)phenol | 441.2 |
| 9.21 | 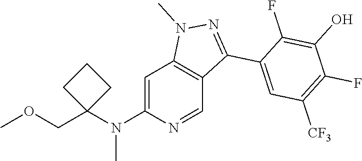 [SMILES: COCC1(N(C)c2cc3c(cn2)c(-c2cc(C(F)(F)F)c(F)c(O)c2F)nn3C)CCC1] | 2,6-Difluoro-3-(6-((1- (methoxymethyl)cyclobutyl)(methyl) amino)-1-methyl-1H-pyrazolo[4,3- c]pyridin-3-yl)-5- (trifluoromethyl)phenol | 457.1 |
| 9.22 | 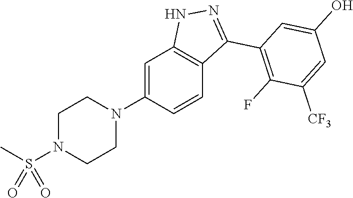 [SMILES: CS(=O)(=O)N1CCN(c2ccc3c(-c4cc(O)cc(C(F)(F)F)c4F)n[nH]c3c2)CC1] | 4-Fluoro-3-(6-(4- (methylsulfonyl)piperazin-1-yl)-1H- indazol-3-yl)-5- (trifluoromethyl)phenol | 459.0 |
| 9.23 (2) | 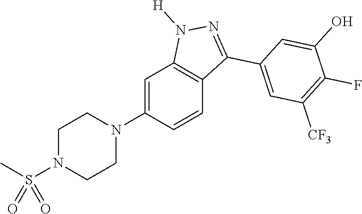 [SMILES: CS(=O)(=O)N1CCN(c2ccc3c(-c4cc(O)c(F)c(C(F)(F)F)c4)n[nH]c3c2)CC1] | 2-Fluoro-5-(6-(4- (methylsulfonyl)piperazin-1-yl)-1H- indazol-3-yl)-3- (trifluoromethyl)phenol | 458.9 |
| 9.24 (2) | 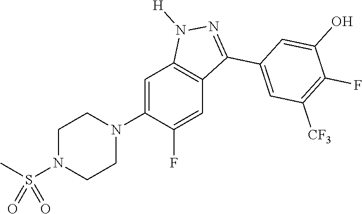 [SMILES: CS(=O)(=O)N1CCN(c2cc3[nH]nc(-c4cc(O)c(F)c(C(F)(F)F)c4)c3cc2F)CC1] | 2-Fluoro-5-(5-fluoro-6-(4- (methylsulfonyl)piperazin-1-yl)-1H- indazol-3-yl)-3- (trifluoromethyl)phenol | 477.0 |
| 9.25 (2) | 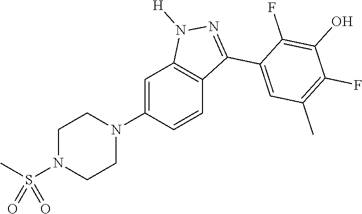 [SMILES: Cc1cc(-c2n[nH]c3cc(N4CCN(S(C)(=O)=O)CC4)ccc23)c(F)c(O)c1F] | 2,6-Difluoro-3-methyl-5-(6-(4- (methylsulfonyl)piperazin-1-yl)-1H- indazol-3-yl)phenol | 423.2 |
| 9.26 (2) | 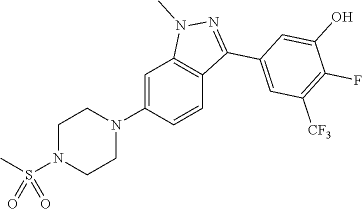 [SMILES: Cn1nc(-c2cc(O)c(F)c(C(F)(F)F)c2)c2ccc(N3CCN(S(C)(=O)=O)CC3)cc21] | 2-Fluoro-5-(1-methyl-6-(4- (methylsulfonyl)piperazin-1-yl)-1H- indazol-3-yl)-3- (trifluoromethyl)phenol | 473.1 |
| 9.27 | 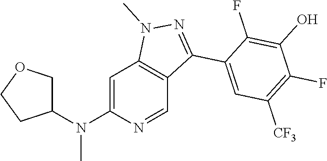 [SMILES: CN(c1cc2c(cn1)c(-c1cc(C(F)(F)F)c(F)c(O)c1F)nn2C)C1CCOC1] | 2,6-Difluoro-3-(1-methyl-6- (methyl(tetrahydrofuran-3-yl)amino)- 1H-pyrazolo[4,3-c]pyridin-3-yl)-5- (trifluoromethyl)phenol | 429.1 |
| 9.28 | 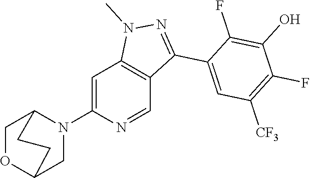 [SMILES: Cn1nc(-c2cc(C(F)(F)F)c(F)c(O)c2F)c2cnc(N3CC4CCC3CO4)cc21] | 3-(6-(2-Oxa-5- azabicyclo[2.2.2]octan-5-yl)-1- methyl-1H-pyrazolo[4,3-c]pyridin-3- yl)-2,6-difluoro-5- (trifluoromethyl)phenol | 441.2 |
| 9.29 | 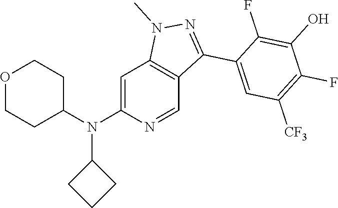 [SMILES: Cn1nc(-c2cc(C(F)(F)F)c(F)c(O)c2F)c2cnc(N(C3CCC3)C3CCOCC3)cc21] | 3-(6-(Cyclobutyl(tetrahydro-2H- pyran-4-yl)amino)-1-methyl-1H- pyrazolo[4,3-c]pyridin-3-yl)-2,6- difluoro-5-(trifluoromethyl)phenol | 483.2 |
| 9.30 | 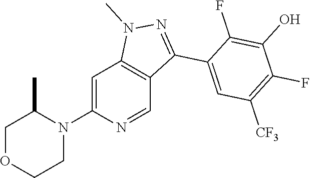 [SMILES: C[C@@H]1COCCN1c1cc2c(cn1)c(-c1cc(C(F)(F)F)c(F)c(O)c1F)nn2C] | (R)-2,6-Difluoro-3-(1-methyl-6-(3- methylmorpholino)-1H-pyrazolo[4,3- c]pyridin-3-yl)-5- (trifluoromethyl)phenol | 429.3 |
| 9.31 | 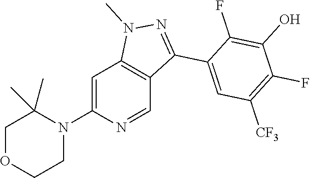 [SMILES: Cn1nc(-c2cc(C(F)(F)F)c(F)c(O)c2F)c2cnc(N3CCOCC3(C)C)cc21] | 3-(6-(3,3-Dimethylmorpholino)-1- methyl-1H-pyrazolo[4,3-c]pyridine- 3-yl)-2,6-difluoro-5- (trifluoromethyl)phenol | 443.3 |
| 9.32 | 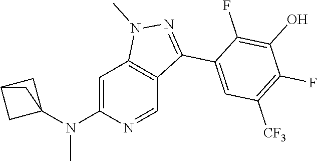 [SMILES: CN(c1cc2c(cn1)c(-c1cc(C(F)(F)F)c(F)c(O)c1F)nn2C)C12CC(C1)C2] | 3-(6-(Bicyclo[1.1.1]pentan-1- yl(methyl)amino)-1-methyl-1H- pyrazolo[4,3-c]pyridin-3-yl)-2,6- difluoro-5-(trifluoromethyl)phenol | 425.3 |
| 9.33 | 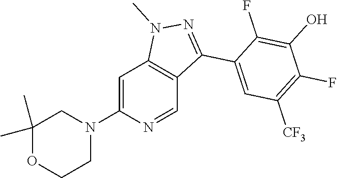 [SMILES: Cn1nc(-c2cc(C(F)(F)F)c(F)c(O)c2F)c2cnc(N3CCOC(C)(C)C3)cc21] | 3-(6-(2,2-Dimethylmorpholino)-1- methyl-1H-pyrazolo[4,3-c]pyridin-3- yl)-2,6-difluoro-5- (trifluoromethyl)phenol | 443.4 |
| 9.34 | 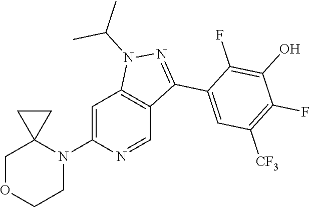 [SMILES: CC(C)n1nc(-c2cc(C(F)(F)F)c(F)c(O)c2F)c2cnc(N3CCOCC34CC4)cc21] | 2,6-Difluoro-3-(1-isopropyl-6-(7- oxa-4-azaspiro[2.5 ]octan-4-yl)-1H- pyrazolo[4,3-c]pyridin-3-yl)-5- (trifluoromethyl)phenol | 469.4 |
| 9.35 | 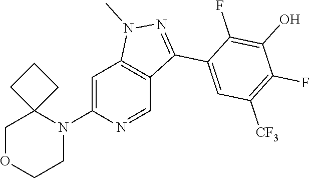 [SMILES: Cn1nc(-c2cc(C(F)(F)F)c(F)c(O)c2F)c2cnc(N3CCOCC34CCC4)cc21] | 2,6-Difluoro-3-(1-methyl-6-(8-oxa-5- azaspiro[3.5]nonan-5-yl)-1H- pyrazolo[4,3-c]pyridin-3-yl)-5- (trifluoromethyl)phenol | 455.4 |
| 9.36 | 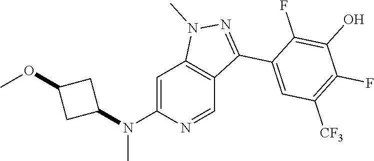 | 2,6-Difluoro-3-(6-((cis-3- methoxycyclobutyl)(methyl)amino)- 1-methyl-1H-pyrazolo[4,3-c]pyridin- 3-yl)-5-(trifluoromethyl)phenol | 443.3 |
| 9.37 | 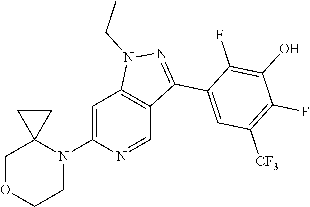 [SMILES: CCn1nc(-c2cc(C(F)(F)F)c(F)c(O)c2F)c2cnc(N3CCOCC34CC4)cc21] | 3-(1-Ethyl-6-(7-oxa-4- azaspiro[2.5]octan-4-yl)-1H- pyrazolo[4,3-c]pyridin-3-yl)-2,6- difluoro-5-(trifluoromethyl)phenol | 455.4 |
| 9.38 | 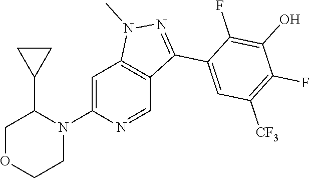 [SMILES: Cn1nc(-c2cc(C(F)(F)F)c(F)c(O)c2F)c2cnc(N3CCOCC3C3CC3)cc21] | 3-(6-(3-Cyclopropylmorpholino)-1- methyl-1H-pyrazolo[4,3-c]pyridin-3- yl)-2,6-difluoro-5- (trifluoromethyl)phenol | 455.4 |
| 9.39 | 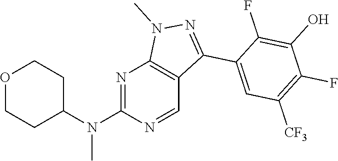 [SMILES: CN(c1ncc2c(-c3cc(C(F)(F)F)c(F)c(O)c3F)nn(C)c2n1)C1CCOCC1] | 2,6-Difluoro-3-(1-methyl-6- (methyl(tetrahydro-2H-pyran-4- yl)amino)-1H-pyrazolo[3,4- d]pyrimidin-3-yl)-5- (trifluoromethyl)phenol | 444.2 |
| 9.40 | 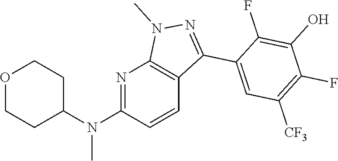 [SMILES: CN(c1ccc2c(-c3cc(C(F)(F)F)c(F)c(O)c3F)nn(C)c2n1)C1CCOCC1] | 2,6-Difluoro-3-(1-methyl-6- (methyl(tetrahydro-2H-pyran-4- yl)amino)-1H-pyrazolo[3,4- b]pyridin-3-yl)-5- (trifluoromethyl)phenol | 443.2 |
| 9.41 | 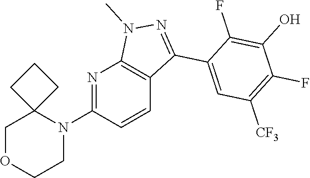 [SMILES: Cn1nc(-c2cc(C(F)(F)F)c(F)c(O)c2F)c2ccc(N3CCOCC34CCC4)nc21] | 2,6-Difluoro-3-(1-methyl-6-(8-oxa-5- azaspiro[3.5]nonan-5-yl)-1H- pyrazolo[3,4-b]pyridin-3-yl)-5- (trifluoromethyl)phenol | 455.1 |
| 9.42 | 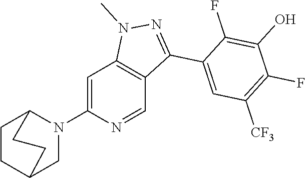 [SMILES: Cn1nc(-c2cc(C(F)(F)F)c(F)c(O)c2F)c2cnc(N3CC4CCC3CC4)cc21] | 3-(6-(2-Azabicyclo[2.2.2]octan-2-yl)- 1-methyl-1H-pyrazolo[4,3-c]pyridin- 3-yl)-2,6-difluoro-5- (trifluoromethyl)phenol | 439.4 |
| 9.43 | 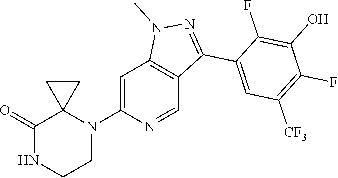 [SMILES: Cn1nc(-c2cc(C(F)(F)F)c(F)c(O)c2F)c2cnc(N3CCNC(=O)C34CC4)cc21] | 4-(3-(2,4-Difluoro-3-hydroxy-5- (trifluoromethyl)phenyl)-1-methyl- 1H-pyrazolo[4,3-c]pyridin-6-yl)-4,7- diazaspiro[2.5]octan-8-one | 454.2 |
| 9.44 | 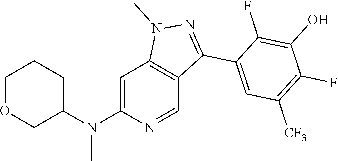 [SMILES: CN(c1cc2c(cn1)c(-c1cc(C(F)(F)F)c(F)c(O)c1F)nn2C)C1CCCOC1] | 2,6-Difluoro-3-(1-methyl-6- (methyl(tetrahydro-2H-pyran-3- yl)amino)-1H-pyrazolo[4,3- c]pyridin-3-yl)-5- (trifluoromethyl)phenol | 443.4 |
| 9.45 | 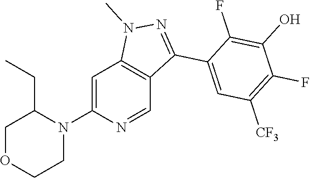 [SMILES: CCC1COCCN1c1cc2c(cn1)c(-c1cc(C(F)(F)F)c(F)c(O)c1F)nn2C] | 3-(6-(3-Ethylmorpholino)-1-methyl- 1H-pyrazolo[4,3-c]pyridin-3-yl)-2,6- difluoro-5-(trifluoromethyl)phenol | 443.4 |
| 9.46 | 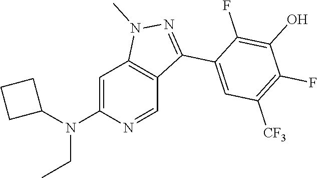 [SMILES: CCN(c1cc2c(cn1)c(-c1cc(C(F)(F)F)c(F)c(O)c1F)nn2C)C1CCC1] | 3-(6-(Cyclobutyl(ethyl)amino)-1- methyl-1H-pyrazolo[4,3-c]pyridin-3- yl)-2,6-difluoro-5- (trifluoromethyl)phenol | 427.3 |
| 9.47 | 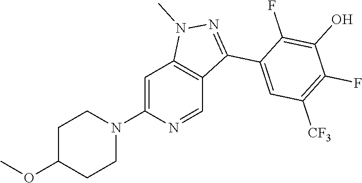 [SMILES: COC1CCN(c2cc3c(cn2)c(-c2cc(C(F)(F)F)c(F)c(O)c2F)nn3C)CC1] | 2,6-Difluoro-3-(6-(4- methoxypiperidin-1-yl)-1-methyl-1H- pyrazolo[4,3-c]pyridin-3-yl)-5- (trifluoromethyl)phenol | 443.3 |
| 9.48 | 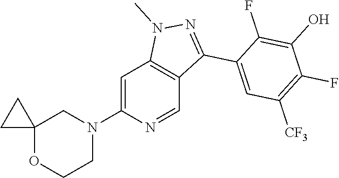 [SMILES: Cn1nc(-c2cc(C(F)(F)F)c(F)c(O)c2F)c2cnc(N3CCOC4(CC4)C3)cc21] | 2,6-Difluoro-3-(1-methyl-6-(4-oxa-7- azaspiro[2.5 ]octan-7-yl)-1H- pyrazolo[4,3-c]pyridin-3-yl)-5- (trifluoromethyl)phenol | 441.3 |
| 9.49 | 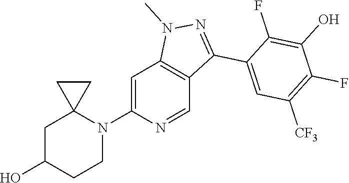 [SMILES: Cn1nc(-c2cc(C(F)(F)F)c(F)c(O)c2F)c2cnc(N3CCC(O)CC34CC4)cc21] | 4-(3-(2,4-Difluoro-3-hydroxy-5- (trifluoromethyl)phenyl)-1-methyl- 1H-pyrazolo[4,3-c]pyridin-6-yl)-4- azaspiro [2.5]octan-7-ol | 455.1 |
| 9.50 | 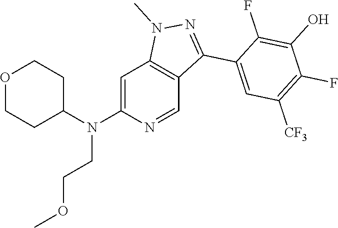 [SMILES: COCCN(c1cc2c(cn1)c(-c1cc(C(F)(F)F)c(F)c(O)c1F)nn2C)C1CCOCC1] | 2,6-Difluoro-3-(6-((2- methoxyethyl)(tetrahydro-2H-pyran- 4-yl)amino)-1-methyl-1H- pyrazolo[4,3-c]pyridin-3-yl)-5- (trifluoromethyl)phenol | 487.3 |
| 9.51 | 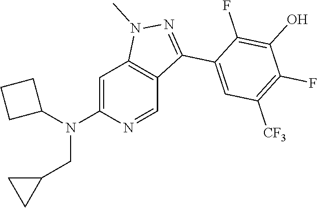 [SMILES: Cn1nc(-c2cc(C(F)(F)F)c(F)c(O)c2F)c2cnc(N(CC3CC3)C3CCC3)cc21] | 3-(6- (Cyclobutyl(cyclopropylmethyl) amino)-1-methyl-1H-pyrazolo[4,3- c]pyridin-3-yl)-2,6-difluoro-5- (trifluoromethyl)phenol | 453.2 |
| 9.52 | 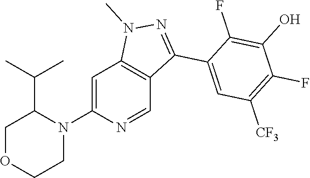 [SMILES: CC(C)C1COCCN1c1cc2c(cn1)c(-c1cc(C(F)(F)F)c(F)c(O)c1F)nn2C] | 2,6-Difluoro-3-(6-(3- isopropylmorpholino)-1-methyl-1H- pyrazolo[4,3-c]pyridin-3-yl)-5- (trifluoromethyl)phenol | 457.4 |
| 9.53 | 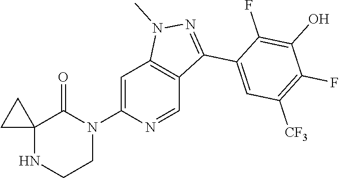 [SMILES: Cn1nc(-c2cc(C(F)(F)F)c(F)c(O)c2F)c2cnc(N3CCNC4(CC4)C3=O)cc21] | 7-(3-(2,4-Difluoro-3-hydroxy-5- (trifluoromethyl)phenyl)-1-methyl- 1H-pyrazolo[4,3-c]pyridin-6-yl)-4,7- diazaspiro[2.5]octan-8-one | 454.2 |
| 9.54 | 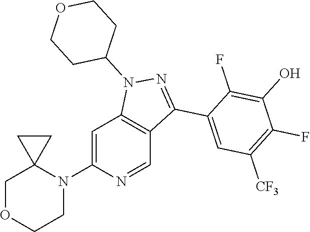 [SMILES: Oc1c(F)c(-c2nn(C3CCOCC3)c3cc(N4CCOCC45CC5)ncc23)cc(C(F)(F)F)c1F] | 3-(6-(7-Oxa-4-azaspiro[2.5]octan-4- yl)-1-(tetrahydro-2H-pyran-4-yl)-1H- pyrazolo[4,3-c]pyridin-3-yl)-2,6- difluoro-5-(trifluoromethyl)phenol | 511.2 |
| 9.55 | 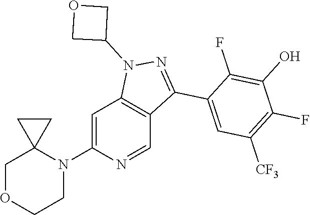 [SMILES: Oc1c(F)c(-c2nn(C3COC3)c3cc(N4CCOCC45CC5)ncc23)cc(C(F)(F)F)c1F] | 2,6-Difluoro-3-(1-(oxetan-3-yl)-6-(7- oxa-4-azaspiro [2.5]octan-4-yl)-1H- pyrazolo[4,3-c]pyridin-3-yl)-5- (trifluoromethyl)phenol | 483.3 |
| 9.56 | 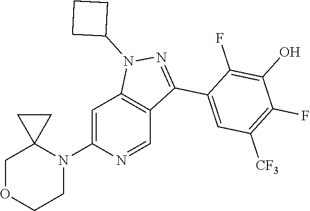 [SMILES: Oc1c(F)c(-c2nn(C3CCC3)c3cc(N4CCOCC45CC5)ncc23)cc(C(F)(F)F)c1F] | 3-(1-Cyclobutyl-6-(7-oxa-4- azaspiro[2.5]octan-4-yl)-1H- pyrazolo[4,3-c]pyridin-3-yl)-2,6- difluoro-5-(trifluoromethyl)phenol | 481.4 |
| 9.57 | 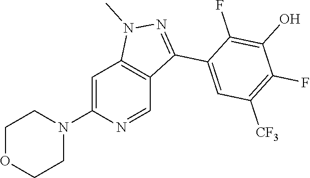 [SMILES: Cn1nc(-c2cc(C(F)(F)F)c(F)c(O)c2F)c2cnc(N3CCOCC3)cc21] | 2,6-Difluoro-3-(1-methyl-6- morpholino-1H-pyrazolo[4,3- c]pyridin-3-yl)-5- (trifluoromethyl)phenol | 415.2 |
| 9.58 | 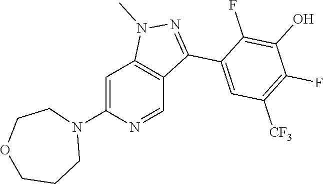 [SMILES: Cn1nc(-c2cc(C(F)(F)F)c(F)c(O)c2F)c2cnc(N3CCCOCC3)cc21] | 2,6-Difluoro-3-(1-methyl-6-(1,4- oxazepan-4-yl)-1H-pyrazolo[4,3- c]pyridin-3-yl)-5- (trifluoromethyl)phenol | 429.1 |
| 9.59 | 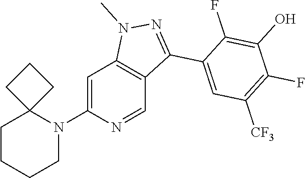 [SMILES: Cn1nc(-c2cc(C(F)(F)F)c(F)c(O)c2F)c2cnc(N3CCCCC34CCC4)cc21] | 2,6-Difluoro-3-(1-methyl-6-(5- azaspiro[3.5]nonan-5-yl)-1H- pyrazolo[4,3-c]pyridin-3-yl)-5- (trifluoromethyl)phenol | 453.3 |
| 9.60 | 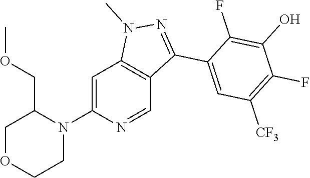 [SMILES: COCC1COCCN1c1cc2c(cn1)c(-c1cc(C(F)(F)F)c(F)c(O)c1F)nn2C] | 2,6-Difluoro-3-(6-(3- (methoxymethyl)morpholino)-1- methyl-1H-pyrazolo[4,3-c]pyridin-3- yl)-5-(trifluoromethyl)phenol | 459.3 |
| 9.61 | 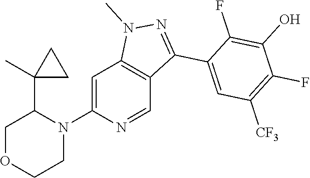 [SMILES: Cn1nc(-c2cc(C(F)(F)F)c(F)c(O)c2F)c2cnc(N3CCOCC3C3(C)CC3)cc21] | 2,6-Difluoro-3-(1-methyl-6-(3-(1- methylcyclopropyl)morpholino)-1H- pyrazolo[4,3-c]pyridin-3-yl)-5- (trifluoromethyl)phenol | 469.4 |
| 9.62 | 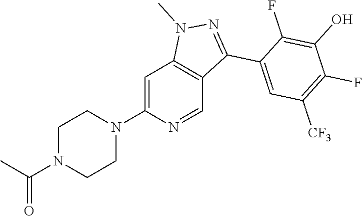 [SMILES: CC(=O)N1CCN(c2cc3c(cn2)c(-c2cc(C(F)(F)F)c(F)c(O)c2F)nn3C)CC1] | 1-(4-(3-(2,4-Difluoro-3-hydroxy-5- (trifluoromethyl)phenyl)-1-methyl- 1H-pyrazolo[4,3-c]pyridin-6- yl)piperazin-1-yl)ethanone | 456.3 |
| 9.63 | 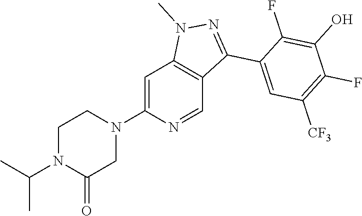 [SMILES: CC(C)N1CCN(c2cc3c(cn2)c(-c2cc(C(F)(F)F)c(F)c(O)c2F)nn3C)CC1=O] | 4-(3-(2,4-Difluoro-3-hydroxy-5- (trifluoromethyl)phenyl)-1-methyl- 1H-pyrazolo[4,3-c]pyridin-6-yl)-1- isopropylpiperazin-2-one | 470.1 |
| 9.64 | 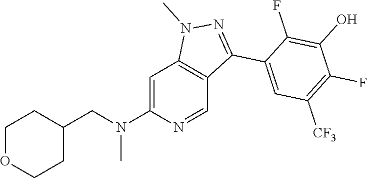 [SMILES: CN(CC1CCOCC1)c1cc2c(cn1)c(-c1cc(C(F)(F)F)c(F)c(O)c1F)nn2C] | 2,6-Difluoro-3-(1-methyl-6- (methyl((tetrahydro-2H-pyran-4- yl)methyl)amino)-1H-pyrazolo[4,3- c]pyridin-3-yl)-5- (trifluoromethyl)phenol | 457.4 |
| 9.65 | 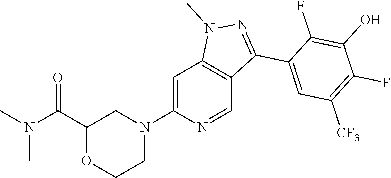 [SMILES: CN(C)C(=O)C1CN(c2cc3c(cn2)c(-c2cc(C(F)(F)F)c(F)c(O)c2F)nn3C)CCO1] | 4-(3-(2,4-Difluoro-3-hydroxy-5- (trifluoromethyl)phenyl)-1-methyl- 1H-pyrazolo[4,3-c]pyridin-6-yl)-N,N- dimethylmorpholine-2-carboxamide | 486.3 |
| 9.66 | 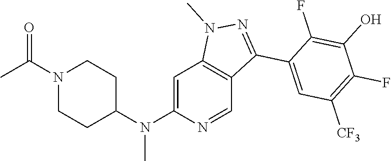 [SMILES: CC(=O)N1CCC(N(C)c2cc3c(cn2)c(-c2cc(C(F)(F)F)c(F)c(O)c2F)nn3C)CC1] | 1-(4-((3-(2,4-Difluoro-3-hydroxy-5- (trifluoromethyl)phenyl)-1-methyl- 1H-pyrazolo[4,3-c]pyridin-6- yl)(methyl)amino)piperidin-1- yl)ethanone | 484.4 |
| 9.67 | 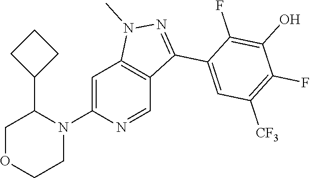 [SMILES: Cn1nc(-c2cc(C(F)(F)F)c(F)c(O)c2F)c2cnc(N3CCOCC3C3CCC3)cc21] | 3-(6-(3-Cyclobutylmorpholino)-1- methyl-1H-pyrazolo[4,3-c]pyridin-3- yl)-2,6-difluoro-5- (trifluoromethyl)phenol | 469.4 |
| 9.68 | 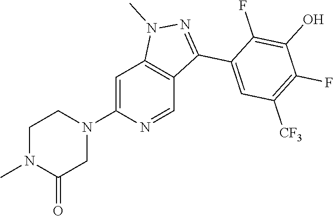 [SMILES: CN1CCN(c2cc3c(cn2)c(-c2cc(C(F)(F)F)c(F)c(O)c2F)nn3C)CC1=O] | 4-(3-(2,4-Difluoro-3-hydroxy-5- (trifluoromethyl)phenyl)-1-methyl- 1H-pyrazolo[4,3-c]pyridin-6-yl)-1- methylpiperazin-2-one | 442.2 |
| 9.69 | 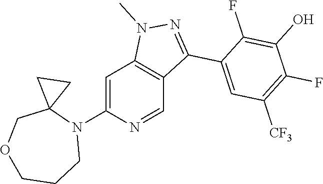 [SMILES: Cn1nc(-c2cc(C(F)(F)F)c(F)c(O)c2F)c2cnc(N3CCCOCC34CC4)cc21] | 2,6-Difluoro-3-(1-methyl-6-(8-oxa-4- azaspiro[2.6]nonan-4-yl)-1H- pyrazolo[4,3-c]pyridin-3-yl)-5- (trifluoromethyl)phenol | 455.2 |
| 9.70 | 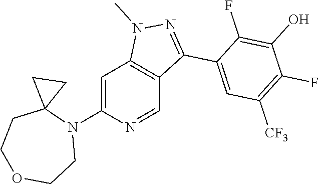 [SMILES: Cn1nc(-c2cc(C(F)(F)F)c(F)c(O)c2F)c2cnc(N3CCOCCC34CC4)cc21] | 2,6-Difluoro-3-(1-methyl-6-(7-oxa-4- azaspiro[2.6]nonan-4-yl)-1H- pyrazolo[4,3-c]pyridin-3-yl)-5- (trifluoromethyl)phenol | 455.2 |
| 9.71 | 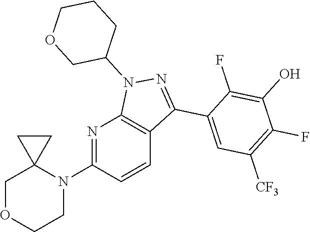 [SMILES: Oc1c(F)c(-c2nn(C3CCCOC3)c3nc(N4CCOCC45CC5)ccc23)cc(C(F)(F)F)c1F] | 3-(6-(7-Oxa-4-azaspiro[2.5]octan-4- yl)-1-(tetrahydro-2H-pyran-3-yl)-1H- pyrazolo[3,4-b]pyridin-3-yl)-2,6- difluoro-5-(trifluoromethyl)phenol | 511.2 |
| 9.72 | 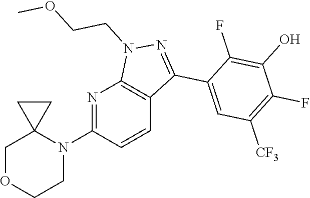 [SMILES: COCCn1nc(-c2cc(C(F)(F)F)c(F)c(O)c2F)c2ccc(N3CCOCC34CC4)nc21] | 2,6-Difluoro-3-(1-(2-methoxyethyl)- 6-(7-oxa-4-azaspiro[2.5]octan-4-yl)- 1H-pyrazolo[3,4-b]pyridin-3-yl)-5- (trifluoromethyl)phenol | 485.1 |
| 9.73 | 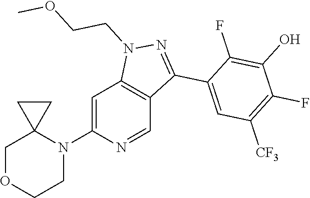 [SMILES: COCCn1nc(-c2cc(C(F)(F)F)c(F)c(O)c2F)c2cnc(N3CCOCC34CC4)cc21] | 2,6-Difluoro-3 -(1-(2-methoxyethyl)- 6-(7-oxa-4-azaspiro[2.5]octan-4-yl)- 1H-pyrazolo[4,3-c]pyridin-3-yl)-5- (trifluoromethyl)phenol | 485.2 |
| 9.74 | 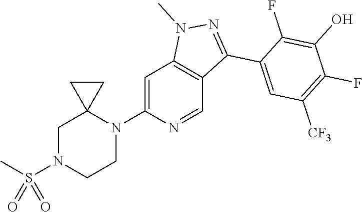 [SMILES: Cn1nc(-c2cc(C(F)(F)F)c(F)c(O)c2F)c2cnc(N3CCN(S(C)(=O)=O)CC34CC4)cc21] | 2,6-Difluoro-3-(1-methyl-6-(7- (methylsulfonyl)-4,7- diazaspiro[2.5]octan-4-yl)-1H- pyrazolo[4,3-c]pyridin-3-yl)-5- (trifluoromethyl)phenol | 518.1 |
| 9.75 | 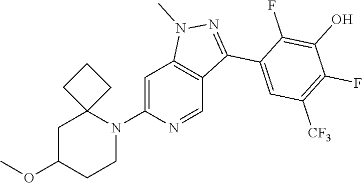 [SMILES: COC1CCN(c2cc3c(cn2)c(-c2cc(C(F)(F)F)c(F)c(O)c2F)nn3C)C2(CCC2)C1] | 2,6-Difluoro-3-(6-(8-methoxy-5- azaspiro[3.5]nonan-5-yl)-1-methyl- 1H-pyrazolo[4,3-c]pyridin-3-yl)-5- (trifluoromethyl)phenol | 483.1 |
| 9.76 | 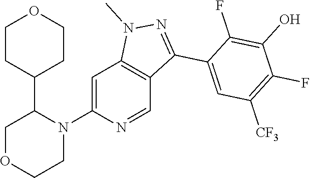 [SMILES: Cn1nc(-c2cc(C(F)(F)F)c(F)c(O)c2F)c2cnc(N3CCOCC3C3CCOCC3)cc21] | 2,6-Difluoro-3-(1-methyl-6-(3- (tetrahydro-2H-pyran-4- yl)morpholino)-1H-pyrazolo[4,3- c]pyridin-3-yl)-5- (trifluoromethyl)phenol | 499.3 |
| 9.77 | 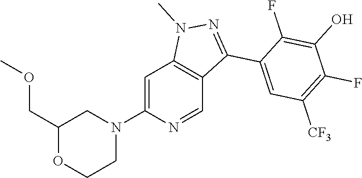 [SMILES: COCC1CN(c2cc3c(cn2)c(-c2cc(C(F)(F)F)c(F)c(O)c2F)nn3C)CCO1] | 2,6-Difluoro-3-(6-(2- (methoxymethyl)morpholino)-1- methyl-1H-pyrazolo[4,3-c]pyridin-3- yl)-5-(trifluoromethyl)phenol | 459.3 |
| 9.78 | 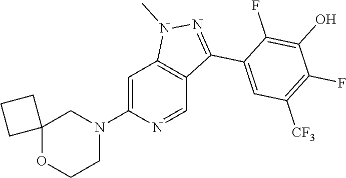 [SMILES: Cn1nc(-c2cc(C(F)(F)F)c(F)c(O)c2F)c2cnc(N3CCOC4(CCC4)C3)cc21] | 2,6-Difluoro-3-(1-methyl-6-(5-oxa-8- azaspiro[3.5]nonan-8-yl)-1H- pyrazolo[4,3-c]pyridin-3-yl)-5- (trifluoromethyl)phenol | 455.3 |
| 9.79 | 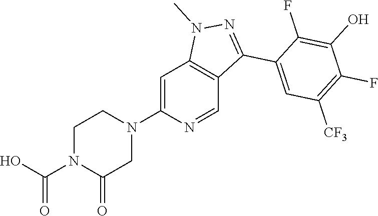 [SMILES: Cn1nc(-c2cc(C(F)(F)F)c(F)c(O)c2F)c2cnc(N3CCN(C(=O)O)C(=O)C3)cc21] | 4-(3-(2,4-Difluoro-3-hydroxy-5- (trifluoromethyl)phenyl)-1-methyl- 1H-pyrazolo[4,3-c]pyridin-6-yl)-2- oxopiperazine-1-carboxylic acid | 472.1 |
| 9.80 | 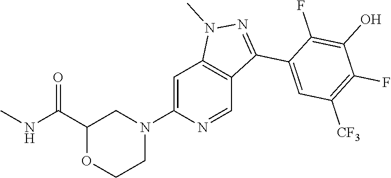 [SMILES: CNC(=O)C1CN(c2cc3c(cn2)c(-c2cc(C(F)(F)F)c(F)c(O)c2F)nn3C)CCO1] | 4-(3-(2,4-Difluoro-3-hydroxy-5- (trifluoromethyl)phenyl)-1-methyl- 1H-pyrazolo[4,3-c]pyridin-6-yl)-N- methylmorpholine-2-carboxamide | 472.2 |
| 9.81 | 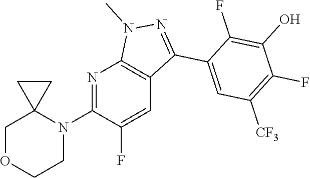 [SMILES: Cn1nc(-c2cc(C(F)(F)F)c(F)c(O)c2F)c2cc(F)c(N3CCOCC34CC4)nc21] | 2,6-Difluoro-3-(5-fluoro-1-methyl-6- (7-oxa-4-azaspiro[2.5]octan-4-yl)- 1H-pyrazolo[3,4-b]pyridin-3-yl)-5- (trifluoromethyl)phenol | 459.1 |
| 9.82 | 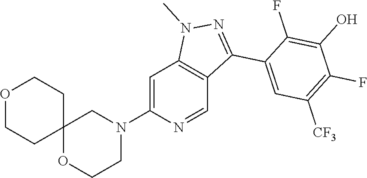 [SMILES: Cn1nc(-c2cc(C(F)(F)F)c(F)c(O)c2F)c2cnc(N3CCOC4(CCOCC4)C3)cc21] | 2,6-Difluoro-3-(1-methyl-6-(1,9- dioxa-4-azaspiro [5.5]undecan-4-yl)- 1H-pyrazolo[4,3-c]pyridin-3-yl)-5- (trifluoromethyl)phenol | 485.3 |
| 9.83 | 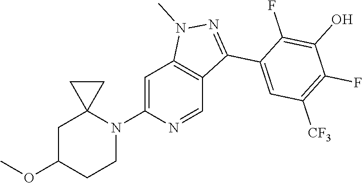 [SMILES: COC1CCN(c2cc3c(cn2)c(-c2cc(C(F)(F)F)c(F)c(O)c2F)nn3C)C2(CC2)C1] | 2,6-Difluoro-3-(6-(7-methoxy-4- azaspiro[2.5]octan-4-yl)-1-methyl- 1H-pyrazolo[4,3-c]pyridin-3-yl)-5- (trifluoromethyl)phenol | 469.3 |
| 9.84 | 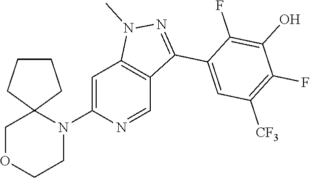 [SMILES: Cn1nc(-c2cc(C(F)(F)F)c(F)c(O)c2F)c2cnc(N3CCOCC34CCCC4)cc21] | 2,6-Difluoro-3-(1-methyl-6-(9-oxa-6- azaspiro[4.5 ]decan-6-yl)-1H- pyrazolo[4,3-c]pyridin-3-yl)-5- (trifluoromethyl)phenol | 469.3 |
| 9.85 | 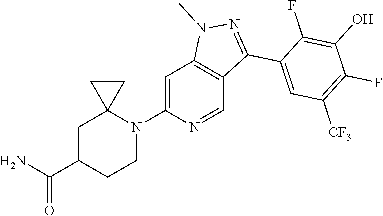 [SMILES: Cn1nc(-c2cc(C(F)(F)F)c(F)c(O)c2F)c2cnc(N3CCC(C(N)=O)CC34CC4)cc21] | 4-(3-(2,4-Difluoro-3-hydroxy-5- (trifluoromethyl)phenyl)-1-methyl- 1H-pyrazolo[4,3-c]pyridin-6-yl)-4- azaspiro[2.5 ]octane-7-carboxamide | 482.1 |
| 9.86 | 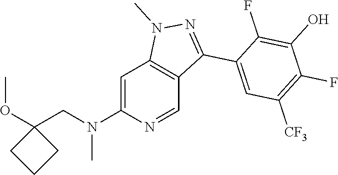 [SMILES: COC1(CN(C)c2cc3c(cn2)c(-c2cc(C(F)(F)F)c(F)c(O)c2F)nn3C)CCC1] | 2,6-Difluoro-3-(6-(((1- methoxycyclobutyl)methyl)(methyl) amino)-1-methyl-1H-pyrazolo[4,3- c]pyridin-3-yl)-5- (trifluoromethyl)phenol | 457.4 |
| 9.87 | 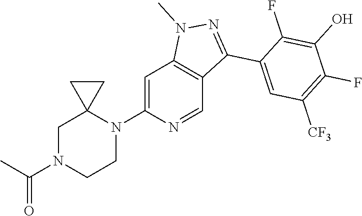 [SMILES: CC(=O)N1CCN(c2cc3c(cn2)c(-c2cc(C(F)(F)F)c(F)c(O)c2F)nn3C)C2(CC2)C1] | 1-(4-(3-(2,4-Difluoro-3-hydroxy-5- (trifluoromethyl)phenyl)-1-methyl- 1H-pyrazolo[4,3-c]pyridin-6-yl)-4,7- diazaspiro[2.5]octan-7-yl)ethanone | 482.1 |
| 9.88 | 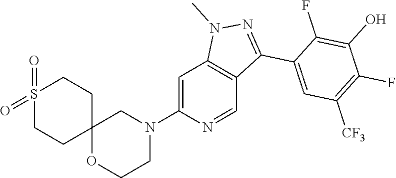 [SMILES: Cn1nc(-c2cc(C(F)(F)F)c(F)c(O)c2F)c2cnc(N3CCOC4(CCS(=O)(=O)CC4)C3)cc21] | 4-(3-(2,4-Difluoro-3-hydroxy-5- (trifluoromethyl)phenyl)-1-methyl- 1H-pyrazolo[4,3-c]pyridin-6-yl)-1- oxa-9-thia-4-azaspiro[5.5]undecane 9,9-dioxide | 533.3 |
| 9.89 | 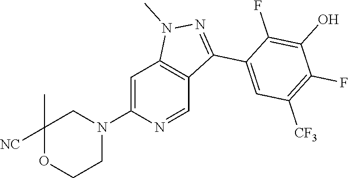 [SMILES: Cn1nc(-c2cc(C(F)(F)F)c(F)c(O)c2F)c2cnc(N3CCOC(C)(C#N)C3)cc21] | 4-(3-(2,4-Difluoro-3-hydroxy-5- (trifluoromethyl)phenyl)-1-methyl- 1H-pyrazolo[4,3-c]pyridin-6-yl)-2- methylmorpholine-2-carbonitrile | 454.2 |
| 9.90 | 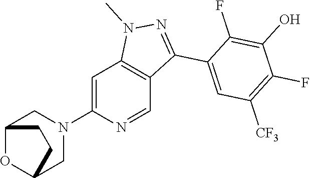 [SMILES: Cn1nc(-c2cc(C(F)(F)F)c(F)c(O)c2F)c2cnc(N3C[C@H]4CC[C@@H](C3)O4)cc21] | 3-(6-(cis-8-Oxa-3- azabicyclo[3.2.1]octan-3-yl)-1- methyl-1H-pyrazolo[4,3-c]pyridin-3- yl)-2,6-difluoro-5- (trifluoromethyl)phenol | 441.3 |
| 9.91 | 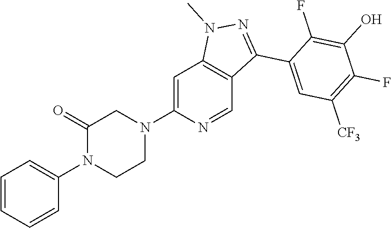 [SMILES: Cn1nc(-c2cc(C(F)(F)F)c(F)c(O)c2F)c2cnc(N3CCN(c4ccccc4)C(=O)C3)cc21] | 4-(3-(2,4-Difluoro-3-hydroxy-5- (trifluoromethyl)phenyl)-1-methyl- 1H-pyrazolo[4,3-c]pyridin-6-yl)-1- phenylpiperazin-2-one | 504.3 |
| 9.92 | 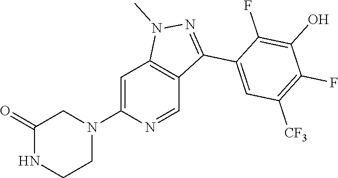 [SMILES: Cn1nc(-c2cc(C(F)(F)F)c(F)c(O)c2F)c2cnc(N3CCNC(=O)C3)cc21] | 4-(3-(2,4-Difluoro-3-hydroxy-5- (trifluoromethyl)phenyl)-1-methyl- 1H-pyrazolo[4,3-c]pyridin-6- yl)piperazin-2-one | 428.0 |
| 9.93 | 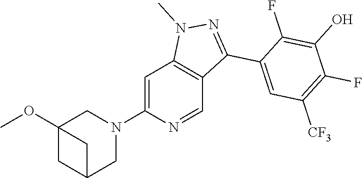 [SMILES: COC12CC(CN(c3cc4c(cn3)c(-c3cc(C(F)(F)F)c(F)c(O)c3F)nn4C)C1)C2] | 2,6-Difluoro-3-(6-(1-methoxy-3- azabicyclo[3.1.1]heptan-3-yl)-1- methyl-1H-pyrazolo[4,3-c]pyridin-3- yl)-5-(trifluoromethyl)phenol | 455.4 |
| 9.94 | 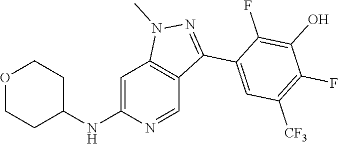 [SMILES: Cn1nc(-c2cc(C(F)(F)F)c(F)c(O)c2F)c2cnc(NC3CCOCC3)cc21] | 2,6-Difluoro-3-(1-methyl-6- ((tetrahydro-2H-pyran-4-yl)amino)- 1H-pyrazolo[4,3-c]pyridin-3-yl)-5- (trifluoromethyl)phenol | 429.1 |
| 9.95 | 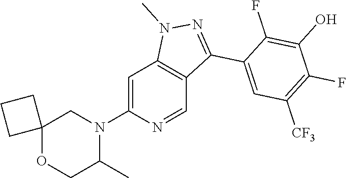 [SMILES: CC1COC2(CCC2)CN1c1cc2c(cn1)c(-c1cc(C(F)(F)F)c(F)c(O)c1F)nn2C] | 2,6-Difluoro-3-(1-methyl-6-(7- methyl-5-oxa-8-azaspiro[3.5]nonan- 8-yl)-1H-pyrazolo[4,3-c]pyridin-3- yl)-5-(trifluoromethyl)phenol | 469.1 |
| 9.96 | 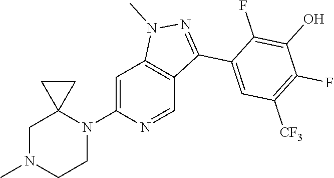 [SMILES: CN1CCN(c2cc3c(cn2)c(-c2cc(C(F)(F)F)c(F)c(O)c2F)nn3C)C2(CC2)C1] | 2,6-Difluoro-3-(1-methyl-6-(8- methyl-5,8-diazaspiro[3.5]nonan-5- yl)-1H-pyrazolo[4,3-c]pyridin-3-yl)- 5-(trifluoromethyl)phenol | 468.2 |
| 9.97 | 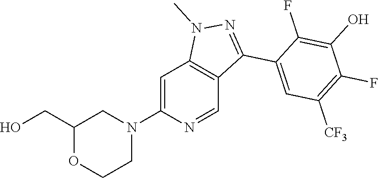 [SMILES: Cn1nc(-c2cc(C(F)(F)F)c(F)c(O)c2F)c2cnc(N3CCOC(CO)C3)cc21] | 2,6-Difluoro-3-(6-(2- (hydroxymethyl)morpholino)-1- methyl-1H-pyrazolo[4,3-c]pyridin-3- yl)-5-(trifluoromethyl)phenol | 445.4 |
| 9.98 | 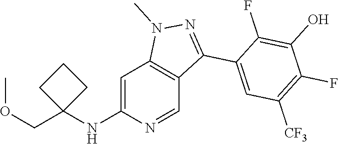 [SMILES: COCC1(Nc2cc3c(cn2)c(-c2cc(C(F)(F)F)c(F)c(O)c2F)nn3C)CCC1] | 2,6-Difluoro-3-(6-((1- (methoxymethyl)cyclobutyl)amino)- 1-methyl-1H-pyrazolo[4,3-c]pyridin- 3-yl)-5-(trifluoromethyl)phenol | 443.6 |
| 9.99 | 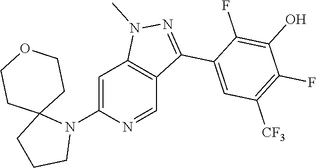 [SMILES: Cn1nc(-c2cc(C(F)(F)F)c(F)c(O)c2F)c2cnc(N3CCCC34CCOCC4)cc21] | 2,6-Difluoro-3-(1-methyl-6-(8-oxa-1- azaspiro[4.5]decan-1-yl)-1H- pyrazolo[4,3-c]pyridin-3-yl)-5- (trifluoromethyl)phenol | 469.7 |
| 9.100 (3) | 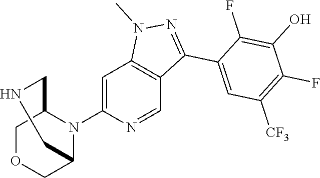 [SMILES: Cn1nc(-c2cc(C(F)(F)F)c(F)c(O)c2F)c2cnc(N3[C@@H]4CNC[C@H]3COC4)cc21] | 3-(6-(cis-3-Oxa-7,9- diazabicyclo[3.3.1 ]nonan-9-yl)-1- methyl-1H-pyrazolo[4,3-c]pyridin-3- yl)-2,6-difluoro-5- (trifluoromethyl)phenol | 456.5 |
| 9.101 (4) | 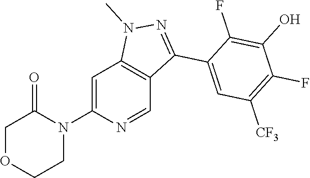 [SMILES: Cn1nc(-c2cc(C(F)(F)F)c(F)c(O)c2F)c2cnc(N3CCOCC3=O)cc21] | 4-(3-(2,4-Difluoro-3-hydroxy-5- (trifluoromethyl)phenyl)-1-methyl- 1H-pyrazolo[4,3-c]pyridin-6- yl)morpholin-3-one | 429.2 |
| 9.102 (4) | 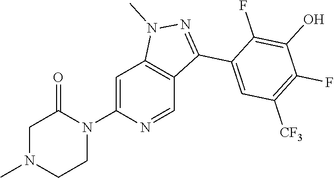 [SMILES: CN1CCN(c2cc3c(cn2)c(-c2cc(C(F)(F)F)c(F)c(O)c2F)nn3C)C(=O)C1] | 1-(3-(2,4-Difluoro-3-hydroxy-5- (trifluoromethyl)phenyl)-1-methyl- 1H-pyrazolo[4,3-c]pyridin-6-yl)-4- methylpiperazin-2-one | 442.6 |
| 9.103 | 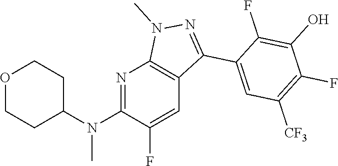 [SMILES: CN(c1nc2c(cc1F)c(-c1cc(C(F)(F)F)c(F)c(O)c1F)nn2C)C1CCOCC1] | 2,6-Difluoro-3-(5-fluoro-1-methyl-6- (methyl(tetrahydro-2H-pyran-4- yl)amino)-1H-pyrazolo[3,4- b]pyridin-3-yl)-5- (trifluoromethyl)phenol | 461.1 |
| 9.104 | 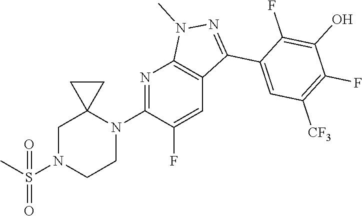 [SMILES: Cn1nc(-c2cc(C(F)(F)F)c(F)c(O)c2F)c2cc(F)c(N3CCN(S(C)(=O)=O)CC34CC4)nc21] | 2,6-Difluoro-3-(5-fluoro-1-methyl-6- (7-(methylsulfonyl)-4,7- diazaspiro[2.5]octan-4-yl)-1H- pyrazolo[3,4-b]pyridin-3-yl)-5- (trifluoromethyl)phenol | 536.1 |
| 9.105 | 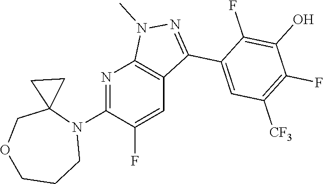 [SMILES: Cn1nc(-c2cc(C(F)(F)F)c(F)c(O)c2F)c2cc(F)c(N3CCCOCC34CC4)nc21] | 2,6-Difluoro-3-(5-fluoro-1-methyl-6- (8-oxa-4-azaspiro[2.6]nonan-4-yl)- 1H-pyrazolo[3,4-b]pyridin-3-yl)-5- (trifluoromethyl)phenol | 473.1 |
| 9.106 | 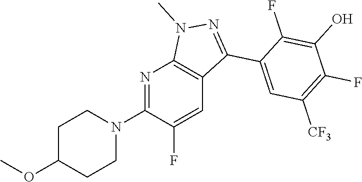 [SMILES: COC1CCN(c2nc3c(cc2F)c(-c2cc(C(F)(F)F)c(F)c(O)c2F)nn3C)CC1] | 2,6-Difluoro-3-(5-fluoro-6-(4- methoxypiperidin-1-yl)-1-methyl-1H- pyrazolo[3,4-b]pyridin-3-yl)-5- (trifluoromethyl)phenol | 461.1 |
| 9.107 | 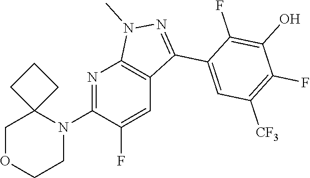 [SMILES: Cn1nc(-c2cc(C(F)(F)F)c(F)c(O)c2F)c2cc(F)c(N3CCOCC34CCC4)nc21] | 2,6-Difluoro-3-(5-fluoro-1-methyl-6- (8-oxa-5-azaspiro[3.5]nonan-5-yl)- 1H-pyrazolo[3,4-b]pyridin-3-yl)-5- (trifluoromethyl)phenol | 473.1 |
| 9.108 | 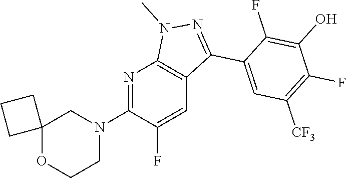 [SMILES: Cn1nc(-c2cc(C(F)(F)F)c(F)c(O)c2F)c2cc(F)c(N3CCOC4(CCC4)C3)nc21] | 2,6-Difluoro-3-(5-fluoro-1-methyl-6- (5-oxa-8-azaspiro[3.5]nonan-8-yl)- 1H-pyrazolo[3,4-b]pyridin-3-yl)-5- (trifluoromethyl)phenol | 473.1 |
| 9.109 (2) | 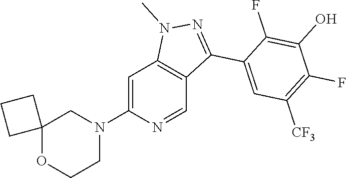 [SMILES: Cn1nc(-c2cc(C(F)(F)F)c(F)c(O)c2F)c2cnc(N3CCOC4(CCC4)C3)cc21] | 2,6-Difluoro-3-(1-methyl-6-(5-oxa-8- azaspiro[3.5]nonan-8-yl)-1H- pyrazolo[4,3-c]pyridin-3-yl)phenol | 387.3 |
| 9.110 | 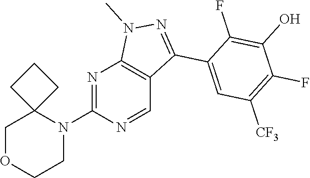 [SMILES: Cn1nc(-c2cc(C(F)(F)F)c(F)c(O)c2F)c2cnc(N3CCOCC34CCC4)nc21] | 2,6-Difluoro-3-(1-methyl-6-(8-oxa-5- azaspiro[3.5]nonan-5-yl)-1H- pyrazolo[3,4-d]pyrimidin-3-yl)-5- (trifluoromethyl)phenol | 456.1 |
| 9.111 (3,6) | 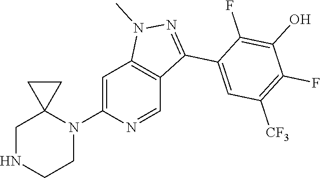 [SMILES: Cn1nc(-c2cc(C(F)(F)F)c(F)c(O)c2F)c2cnc(N3CCNCC34CC4)cc21] | 2,6-Difluoro-3-(1-methyl-6-(4,7- diazaspiro[2.5]octan-4-yl)-1H- pyrazolo[4,3-c]pyridin-3-yl)-5- (trifluoromethyl)phenol | 440.4 |
| 9.112 | 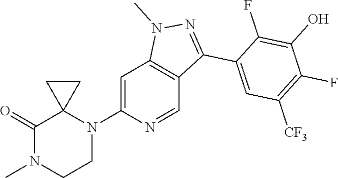 [SMILES: CN1CCN(c2cc3c(cn2)c(-c2cc(C(F)(F)F)c(F)c(O)c2F)nn3C)C2(CC2)C1=O] | 4-(3-(2,4-Difluoro-3-hydroxy-5- (trifluoromethyl)phenyl)-1-methyl- 1H-pyrazolo[4,3-c]pyridin-6-yl)-7- methyl-4,7-diazaspiro [2.5]octan-8- one | 468.7 |
| 9.113 (4) | 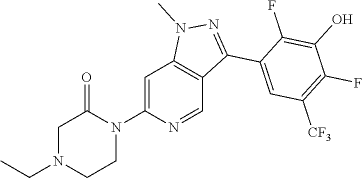 [SMILES: CCN1CCN(c2cc3c(cn2)c(-c2cc(C(F)(F)F)c(F)c(O)c2F)nn3C)C(=O)C1] | 1-(3-(2,4-Difluoro-3-hydroxy-5- (trifluoromethyl)phenyl)-1-methyl- 1H-pyrazolo[4,3-c]pyridin-6-yl)-4- ethylpiperazin-2-one | 456.4 |
| 9.114 (6) | 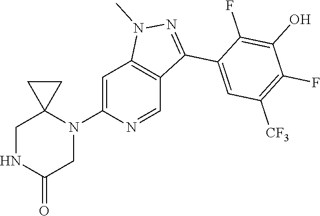 [SMILES: Cn1nc(-c2cc(C(F)(F)F)c(F)c(O)c2F)c2cnc(N3CC(=O)NCC34CC4)cc21] | 4-(3-(2,4-Difluoro-3-hydroxy-5- (trifluoromethyl)phenyl)-1-methyl- 1H-pyrazolo[4,3-c]pyridin-6-yl)-4,7- diazaspiro[2.5]octan-6-one | 454.6 |
| 9.115 (6) | 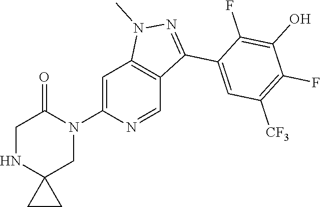 [SMILES: Cn1nc(-c2cc(C(F)(F)F)c(F)c(O)c2F)c2cnc(N3CC4(CC4)NCC3=O)cc21] | 7-(3-(2,4-Difluoro-3-hydroxy-5- (trifluoromethyl)phenyl)-1-methyl- 1H-pyrazolo[4,3-c]pyridin-6-yl)-4,7- diazaspiro[2.5]octan-6-one | 454.3 |
| 9.116 | 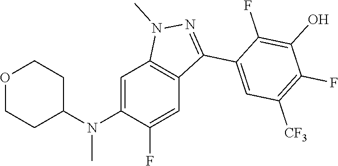 [SMILES: CN(c1cc2c(cc1F)c(-c1cc(C(F)(F)F)c(F)c(O)c1F)nn2C)C1CCOCC1] | 2,6-Difluoro-3-(5-fluoro-1-methyl-6- (methyl(tetrahydro-2H-pyran-4- yl)amino)-1H-indazol-3-yl)-5- (trifluoromethyl)phenol | 460.6 |
| 9.117 (7) | 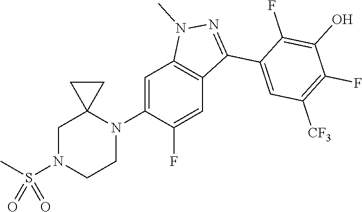 [SMILES: Cn1nc(-c2cc(C(F)(F)F)c(F)c(O)c2F)c2cc(F)c(N3CCN(S(C)(=O)=O)CC34CC4)cc21] | 2,6-Difluoro-3-(5-fluoro-1-methyl-6- (7-(methylsulfonyl)-4,7- diazaspiro[2.5]octan-4-yl)-1H- indazol-3-yl)-5- (trifluoromethyl)phenol | 535.4 |
| 9.118 (1,8) | 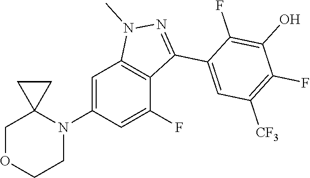 [SMILES: Cn1nc(-c2cc(C(F)(F)F)c(F)c(O)c2F)c2c(F)cc(N3CCOCC34CC4)cc21] | 2,6-Difluoro-3-(4-fluoro-1-methyl-6- (7-oxa-4-azaspiro[2.5]octan-4-yl)- 1H-indazol-3-yl)-5- (trifluoromethyl)phenol | 458.0 |
| 9.119 (1,8) | 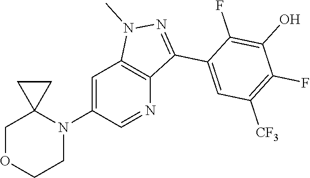 [SMILES: Cn1nc(-c2cc(C(F)(F)F)c(F)c(O)c2F)c2ncc(N3CCOCC34CC4)cc21] | 2,6-Difluoro-3-(1-methyl-6-(7-oxa-4- azaspiro[2.5]octan-4-yl)-1H- pyrazolo[4,3-b]pyridin-3-yl)-5- (trifluoromethyl)phenol | 441.1 |
| 9.120 (1,9) | 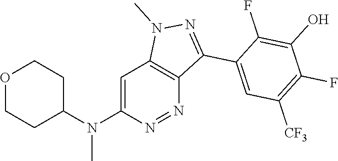 [SMILES: CN(c1cc2c(nn1)c(-c1cc(C(F)(F)F)c(F)c(O)c1F)nn2C)C1CCOCC1] | 2,6-Difluoro-3-(1-methyl-6- (methyl(tetrahydro-2H-pyran-4- yl)amino)-1H-pyrazolo[4,3- c]pyridazin-3-yl)-5- (trifluoromethyl)phenol | 444.1 |
| 9.121 (10) | 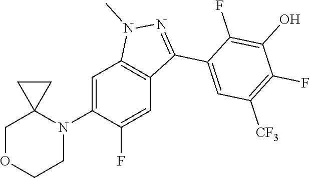 [SMILES: Cn1nc(-c2cc(C(F)(F)F)c(F)c(O)c2F)c2cc(F)c(N3CCOCC34CC4)cc21] | 2,6-Difluoro-3-(5-fluoro-1-methyl-6- (7-oxa-4-azaspiro[2.5]octan-4-yl)- 1H-indazol-3-yl)-5- (trifluoromethyl)phenol | 458.3 |
| 9.122 | 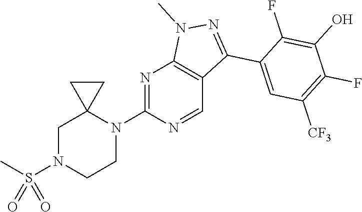 [SMILES: Cn1nc(-c2cc(C(F)(F)F)c(F)c(O)c2F)c2cnc(N3CCN(S(C)(=O)=O)CC34CC4)nc21] | 2,6-Difluoro-3-(1-methyl-6-(7- (methylsulfonyl)-4,7- diazaspiro[2.5]octan-4-yl)-1H- pyrazolo[3,4-d]pyrimidin-3-yl)-5- (trifluoromethyl)phenol | 519.4 |
| 9.123 | 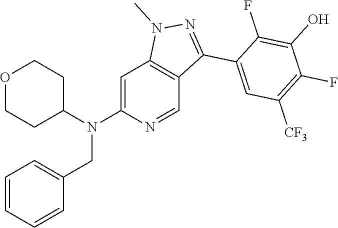 [SMILES: Cn1nc(-c2cc(C(F)(F)F)c(F)c(O)c2F)c2cnc(N(Cc3ccccc3)C3CCOCC3)cc21] | 3-(6-(Benzyl(tetrahydro-2H-pyran-4- yl)amino)-1-methyl-1H-pyrazolo[4,3- c]pyridin-3-yl)-2,6-difluoro-5- (trifluoromethyl)phenol | 519.4 |
| 9.124 | 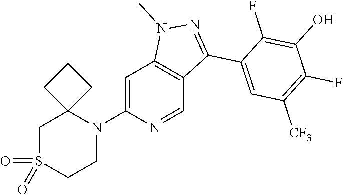 [SMILES: Cn1nc(-c2cc(C(F)(F)F)c(F)c(O)c2F)c2cnc(N3CCS(=O)(=O)CC34CCC4)cc21] | 5-(3-(2,4-Difluoro-3-hydroxy-5- (trifluoromethyl)phenyl)-1-methyl- 1H-pyrazolo[4,3-c]pyridin-6-yl)-8- thia-5-azaspiro[3.5]nonane 8,8- dioxide | 503.5 |
| 9.125 | 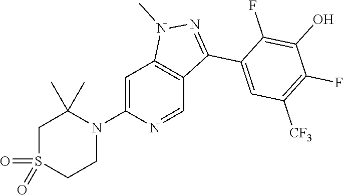 [SMILES: Cn1nc(-c2cc(C(F)(F)F)c(F)c(O)c2F)c2cnc(N3CCS(=O)(=O)CC3(C)C)cc21] | 4-(3-(2,4-Difluoro-3-hydroxy-5- (trifluoromethyl)phenyl)-1-methyl- 1H-pyrazolo[4,3-c]pyridin-6-yl)-3,3- dimethylthiomorpholine 1,1-dioxide | 491.3 |
| 9.126 | 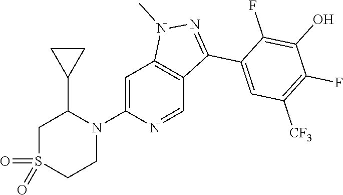 [SMILES: Cn1nc(-c2cc(C(F)(F)F)c(F)c(O)c2F)c2cnc(N3CCS(=O)(=O)CC3C3CC3)cc21] | 3-Cyclopropyl-4-(3-(2,4-difluoro-3- hydroxy-5-(trifluoromethyl)phenyl)- 1-methyl-1H-pyrazolo[4,3-c]pyridin- 6-yl)thiomorpholine 1,1-dioxide | 503.3 |
| 9.127 (11) | 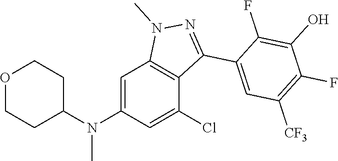 [SMILES: CN(c1cc(Cl)c2c(-c3cc(C(F)(F)F)c(F)c(O)c3F)nn(C)c2c1)C1CCOCC1] | 3-(4-Chloro-1-methyl-6- (methyl(tetrahydro-2H-pyran-4- yl)amino)-1H-indazol-3-yl)-2,6- difluoro-5-(trifluoromethyl)phenol | 476.0 |
| 9.128 | 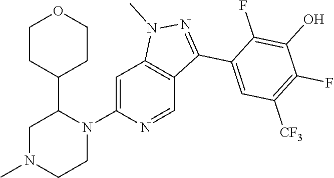 [SMILES: CN1CCN(c2cc3c(cn2)c(-c2cc(C(F)(F)F)c(F)c(O)c2F)nn3C)C(C2CCOCC2)C1] | 2,6-Difluoro-3-(1-methyl-6-(4- methyl-2-(tetrahydro-2H-pyran-4- yl)piperazin-1-yl)-1H-pyrazolo[4,3- c]pyridin-3-yl)-5- (trifluoromethyl)phenol | 512.5 |
| 9.129 | 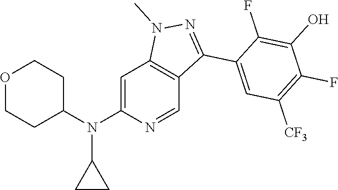 [SMILES: Cn1nc(-c2cc(C(F)(F)F)c(F)c(O)c2F)c2cnc(N(C3CCOCC3)C3CC3)cc21] | 3-(6-(Cyclopropyl(tetrahydro-2H- pyran-4-yl)amino)-1-methyl-1H- pyrazolo[4,3-c]pyridin-3-yl)-2,6- difluoro-5-(trifluoromethyl)phenol | 469.6 |
| 9.130 | 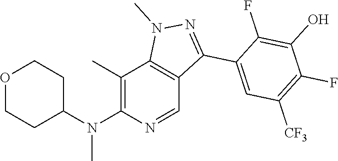 [SMILES: Cc1c(N(C)C2CCOCC2)ncc2c(-c3cc(C(F)(F)F)c(F)c(O)c3F)nn(C)c12] | 3-(1,7-Dimethyl-6- (methyl(tetrahydro-2H-pyran-4- yl)amino)-1H-pyrazolo[4,3- c]pyridin-3-yl)-2,6-difluoro-5- (trifluoromethyl)phenol | 457.1 |
| 9.131 | 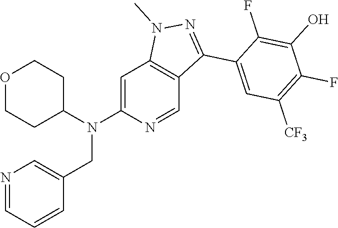 [SMILES: Cn1nc(-c2cc(C(F)(F)F)c(F)c(O)c2F)c2cnc(N(Cc3cccnc3)C3CCOCC3)cc21] | 2,6-Difluoro-3-(1-methyl-6-((pyridin- 3-ylmethyl)(tetrahydro-2H-pyran-4- yl)amino)-1H-pyrazolo[4,3- c]pyridin-3-yl)-5- (trifluoromethyl)phenol | 520.4 |
| 9.132 | 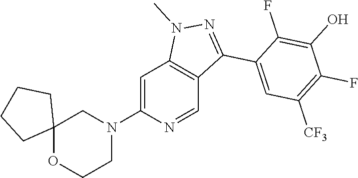 [SMILES: Cn1nc(-c2cc(C(F)(F)F)c(F)c(O)c2F)c2cnc(N3CCOC4(CCCC4)C3)cc21] | 2,6-Difluoro-3-(1-methyl-6-(6-oxa-9- azaspiro[4.5]decan-9-yl)-1H- pyrazolo[4,3-c]pyridin-3-yl)-5- (trifluoromethyl)phenol | 469.4 |
| 9.133 | 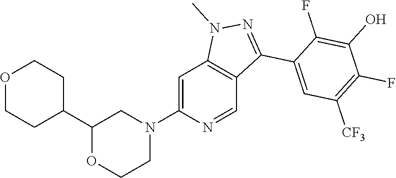 [SMILES: Cn1nc(-c2cc(C(F)(F)F)c(F)c(O)c2F)c2cnc(N3CCOC(C4CCOCC4)C3)cc21] | 2,6-Difluoro-3-(1-methyl-6-(2- (tetrahydro-2H-pyran-4- yl)morpholino)-1H-pyrazolo[4,3- c]pyridin-3-yl)-5- (trifluoromethyl)phenol | 499.4 |
| 9.134 | 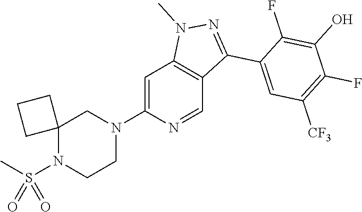 [SMILES: Cn1nc(-c2cc(C(F)(F)F)c(F)c(O)c2F)c2cnc(N3CCN(S(C)(=O)=O)C4(CCC4)C3)cc21] | 2,6-Difluoro-3-(1-methyl-6-(5- (methylsulfonyl)-5,8- diazaspiro[3.5]nonan-8-yl)-1H- pyrazolo[4,3-c]pyridin-3-yl)-5- (trifluoromethyl)phenol | 532.4 |
| 9.135 | 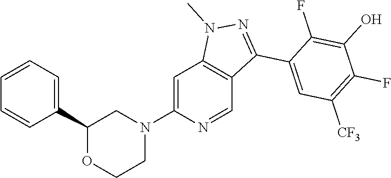 [SMILES: Cn1nc(-c2cc(C(F)(F)F)c(F)c(O)c2F)c2cnc(N3CCO[C@@H](c4ccccc4)C3)cc21] | (S)-2,6-Difluoro-3-(1-methyl-6-(2- phenylmorpholino)-1H-pyrazolo[4,3- c]pyridin-3-yl)-5- (trifluoromethyl)phenol | 491.5 |
| 9.136 (8) | 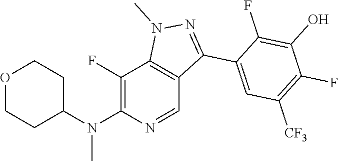 [SMILES: CN(c1ncc2c(-c3cc(C(F)(F)F)c(F)c(O)c3F)nn(C)c2c1F)C1CCOCC1] | 2,6-Difluoro-3-(7-fluoro-1-methyl-6- (methyl(tetrahydro-2H-pyran-4- yl)amino)-1H-pyrazolo[4,3- c]pyridin-3-yl)-5- (trifluoromethyl)phenol | 461.0 |
| 9.137 (9) | 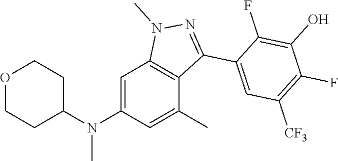 [SMILES: Cc1cc(N(C)C2CCOCC2)cc2c1c(-c1cc(C(F)(F)F)c(F)c(O)c1F)nn2C] | 3-(1,4-Dimethyl-6- (methyl(tetrahydro-2H-pyran-4- yl)amino)-1H-indazol-3-yl)-2,6- difluoro-5-(trifluoromethyl)phenol | 456.1 |
| 9.138 (8) | 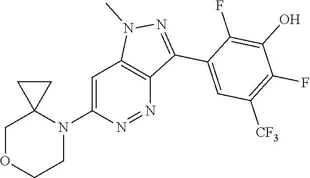 [SMILES: Cn1nc(-c2cc(C(F)(F)F)c(F)c(O)c2F)c2nnc(N3CCOCC34CC4)cc21] | 2,6-Difluoro-3-(1-methyl-6-(7-oxa-4- azaspiro[2.5 ]octan-4-yl)-1H- pyrazolo[4,3-c]pyridazin-3-yl)-5- (trifluoromethyl)phenol | 442.1 |
| 9.139 | 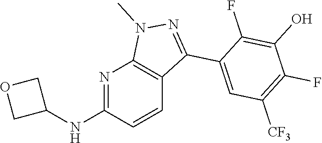 [SMILES: Cn1nc(-c2cc(C(F)(F)F)c(F)c(O)c2F)c2ccc(NC3COC3)nc21] | 2,6-Difluoro-3-(1-methyl-6-(oxetan- 3-ylamino)-1H-pyrazolo[3,4- b]pyridin-3-yl)-5- (trifluoromethyl)phenol | 401.2 |
| 9.140 | 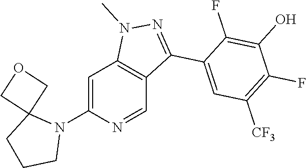 [SMILES: Cn1nc(-c2cc(C(F)(F)F)c(F)c(O)c2F)c2cnc(N3CCCC34COC4)cc21] | 2,6-Difluoro-3-(1-methyl-6-(2-oxa-5- azaspiro[3.4]octan-5-yl)-1H- pyrazolo[4,3-c]pyridin-3-yl)-5- (trifluoromethyl)phenol | 441.2 |
| 9.141 (8,12) | 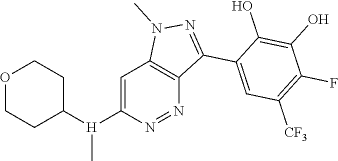 | 3-Fluoro-6-(1-methyl-6- (methyl(tetrahydro-2H-pyran-4- yl)amino)-1H-pyrazolo[4,3- c]pyridazin-3-yl)-4- (trifluoromethyl)benzene-1,2-diol | 442.1 |

Alternate condition used:
1. Dioxane was replaced by PhMe as solvent.
2. No phenol protecting group was used.
3. Boc protected amine was used;
4. XantPhos, Cs₂CO₃, Pd(PPh₃)₄, dioxane, 120° C., 120 min;
6. Isomers separated by silica gel chromatography following the Buchwald conditions and then deprotected separately;
7. BINAP, Cs₂CO₃, Pd₂(dba)₃, toluene, 110° C., overnight;
8. Buchwald: 100° C., 2-3 h or overnight then debenzylation: TFA, 50-70° C., 1-2h;
9. Debenzylation: TFA, 50-70° C., 0.5-2 h then Buchwald: 100° C., 2 h;
10. methanesulfonato(2-bis(3,5-di(trifluoromethyl)phenylphosphino)-3,6-dimethoxy-2″,6″-bis(dimethylamino)-1,1″-biphenyl)(2″-methylamino-1,1″-biphenyl-2-yl)palladium(II), 2-[bis(3,5-trifluoromethylphenylphosphino)-3,6-dimethoxy]-2″,6″-dimethylamino-1,1″-biphenyl, NaOfBu, CPME, 80° C., 2.5 h; 11. XantPhos Pd G4,XantPhos, NaOtBu, t-AmOH, 80° C., 2 h then debenzylation: TFA, DCM, 70° C., 2 h;
12. Synthesized from Intermediate 19: Fluoro was displaced during Buchwald conditions.

### Compound 10

### 2-Fluoro-5-(6-(4-(methylsulfonyl)piperazin-1-yl)-1H-pyrazolo[3,4-d]pyrimidin-3-yl)-3-(trifluoromethyl)phenol

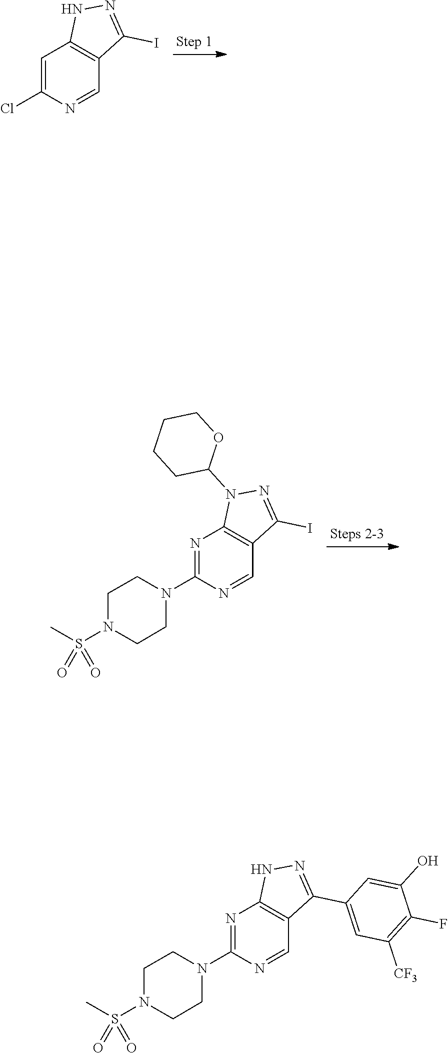 [SMILES: Clc1cc2[nH]nc(I)c2cn1 ; CS(=O)(=O)N1CCN(c2ncc3c(I)nn(C4CCCCO4)c3n2)CC1 ; CS(=O)(=O)N1CCN(c2ncc3c(-c4cc(O)c(F)c(C(F)(F)F)c4)n[nH]c3n2)CC1]

### Step 1: 3-Iodo-6-(4-(methylsulfonyl)piperazin-1-yl)-1-(tetrahydro-2H-pyran-2-yl)-1H-pyrazolo[3,4-d]pyrimidine

2H-3,4-Dihydropyran (0.13 mL, 1.43 mmol) and p-toluenesulfonic acid (14 mg, 0.07 mmol) were added to 6-chloro-3-iodopyrazolo[5,4-d]pyrimidine (0.20 g, 0.71 mmol) in DCM (2.0 mL). The mixture was stirred for 15 h, diluted (DCM), washed (saturated NaHCO₃), and then concentrated. The material was taken up in DMA (2.0 mL). 1-Methanesulfonyl-piperazine (225 mg, 1.37 mmol) and Hunig's base (0.48 mL, 2.74 mmol) were added. The reaction was heated in a microwave at 100° C. for 1 h and partitioned between saturated NaHCO₃ and EtOAc. The aqueous layer was extracted (EtOAc). The combined organics were dried (Na₂SO₄) and concentrated under reduced pressure to give the crude product that was used directly in subsequent reactions (128 mg). LCMS: 492.9 [M+H]⁺.

### Step 2: 3-(3-(Benzyloxy)-4-fluoro-5-(trifluoromethyl)phenyl)-6-(4-(methylsulfonyl)piperazin-1-yl)-1-(tetrahydro-2H-pyran-2-yl)-1H-pyrazolo[3,4-d]pyrimidine

A mixture of 3-iodo-6-(4-(methylsulfonyl)piperazin-1-yl)-1-(tetrahydro-2H-pyran-2-yl)-1H-pyrazolo[3,4-d]pyrimidine (50 mg, 0.10 mmol), 2-fluoro-3-(phenylmethoxy)-5-(4,4,5,5-tetramethyl(1,3,2-dioxaborolan-2-yl))-1-(trifluoromethyl)benzene (48 mg, 0.12 mmol), KF (24 mg, 0.41 mmol), and Pd(dppf)Cl₂ (7.4 mg, 0.010 mmol) in dioxane (2.0 mL) and water (0.5 mL) was purged with nitrogen for 5 min, heated in a microwave at 90° C. for 40 min, diluted with water, and then extracted with EtOAc. The combined organics were dried (Na₂SO₄), concentrated, and then purified by silica gel chromatography (0-100% EtOAc/hexanes) to give 3-(3-(benzyloxy)-4-fluoro-5-(trifluoromethyl)phenyl)-6-(4-(methylsulfonyl)piperazin-1-yl)-1-(tetrahydro-2H-pyran-2-yl)-1H-pyrazolo[3,4-d]pyrimidine (54 mg, 84%) as a white powder. LCMS: 635.1 [M+H]⁺.

### Step 3: 2-Fluoro-5-(6-(4-(methylsulfonyl)piperazin-1-yl)-1H-pyrazolo[3,4-d]pyrimidin-3-yl)-3-(trifluoromethyl)phenol

A mixture of 3-(3-(benzyloxy)-4-fluoro-5-(trifluoromethyl)phenyl)-6-(4-(methylsulfonyl)piperazin-1-yl)-1-(tetrahydro-2H-pyran-2-yl)-1H-pyrazolo[3,4-d]pyrimidine (50 mg, 0.079 mmol), TFA (1 mL), and DCM (1 mL) was stirred at rt for 15 h, concentrated under reduced pressure, and then purified by silica gel chromatography (0-100% EtOAc/hexanes). The residue was taken up in THF (10 mL). Palladium on carbon, (5 wt. % 10 mg) was added. The reaction was stirred under a balloon of hydrogen for 2 h, filtered, and concentrated under reduced pressure. The residue was purified by prep-HPLC to give 2-fluoro-5-(6-(4-(methylsulfonyl)piperazin-1-yl)-1H-pyrazolo[3,4-d]pyrimidin-3-yl)-3-(trifluoromethyl)phenol (21 mg, 58%) as a white powder. ¹H NMR (400 MHz, DMSO-d₆) δ: 13.47 (br s, 1H), 10.90 (br s, 1H), 9.18 (s, 1H), 7.87 (br d, J=7.7 Hz, 1H), 7.62 (br d, J=4.9 Hz, 1H), 3.97 (br s, 4H), 3.23 (br s, 4H), 2.90 (s, 3H); LCMS: 461.0 [M+H]⁺.

The Compounds below were synthesized in a similar manner to that described for Compound 10.

| Cmpd | Structure | Name | [M + H]⁺ |
|---|---|---|---|
| 10.01 | 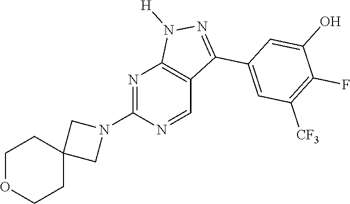 [SMILES: Oc1cc(-c2n[nH]c3nc(N4CC5(CCOCC5)C4)ncc23)cc(C(F)(F)F)c1F] | 5-(6-(7-Oxa-2-azaspiro[3.5 ]nonan-2- yl)-1H-pyrazolo[3,4-d]pyrimidin-3- yl)-2-fluoro-3- (trifluoromethyl)phenol | 424.2 |
| 10.02 | 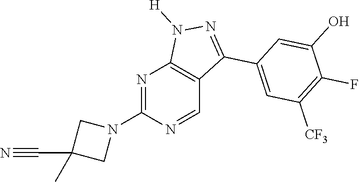 [SMILES: CC1(C#N)CN(c2ncc3c(-c4cc(O)c(F)c(C(F)(F)F)c4)n[nH]c3n2)C1] | 1-(3-(4-Fluoro-3-hydroxy-5- (trifluoromethyl)phenyl)-1H- pyrazolo[3,4-d]pyrimidin-6-yl)-3- methylazetidine-3-carbonitrile | 393.1 |
| 10.03 | 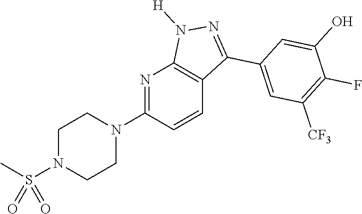 [SMILES: CS(=O)(=O)N1CCN(c2ccc3c(-c4cc(O)c(F)c(C(F)(F)F)c4)n[nH]c3n2)CC1] | 2-Fluoro-5-(6-(4- (methylsulfonyl)piperazin-1-yl)-1H- pyrazolo[3,4-b]pyridin-3-yl)-3- (trifluoromethyl)phenol | 460.2 |
| 10.04 | 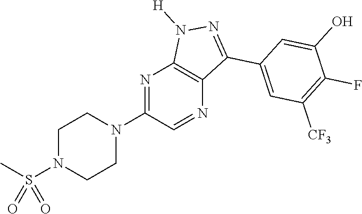 [SMILES: CS(=O)(=O)N1CCN(c2cnc3c(-c4cc(O)c(F)c(C(F)(F)F)c4)n[nH]c3n2)CC1] | 2-Fluoro-5-(6-(4- (methylsulfonyl)piperazin-1-yl)-1H- pyrazolo[3,4-b]pyrazin-3-yl)-3- (trifluoromethyl)phenol | 460.9 |
| 10.05 | 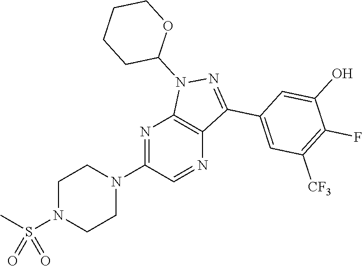 [SMILES: CS(=O)(=O)N1CCN(c2cnc3c(-c4cc(O)c(F)c(C(F)(F)F)c4)nn(C4CCCCO4)c3n2)CC1] | 2-Fluoro-5-(6-(4- (methylsulfonyl)piperazin-1-yl)-1- (tetrahydro-2H-pyran-2-yl)-1H- pyrazolo[3,4-b]pyrazin-3-yl)-3- (trifluoromethyl)phenol | 545.4 |
| 10.06 | 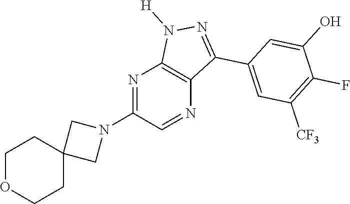 [SMILES: Oc1cc(-c2n[nH]c3nc(N4CC5(CCOCC5)C4)cnc23)cc(C(F)(F)F)c1F] | 5-(6-(7-Oxa-2-azaspiro[3.5]nonan-2- yl)-1H-pyrazolo[3,4-b]pyrazin-3-yl)- 2-fluoro-3-(trifluoromethyl)phenol | 424.1 |

### Compound 11

### 2-Fluoro-3-(6-(4-(methylsulfonyl)piperazin-1-yl)-1H-indazol-3-yl)-5-(trifluoromethyl)phenol

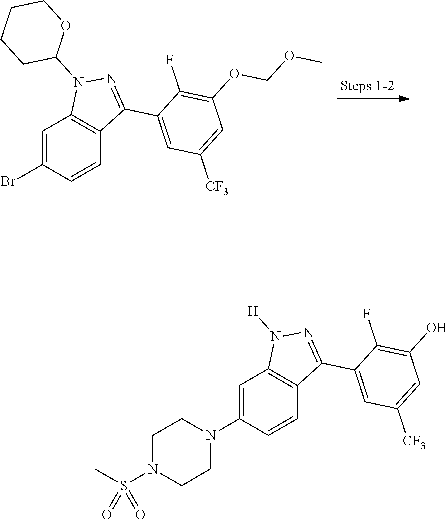

### Step 1: 3-(2-Fluoro-3-(methoxymethoxy)-5-(trifluoromethyl)phenyl)-6-(4-(methylsulfonyl)piperazin-1-yl)-1-(tetrahydro-2H-pyran-2-yl)-1H-indazole

A mixture of Intermediate 18.08 (255 mg, 0.51 mmol), 1-methylsulfonylpiperazine (117 mg, 0.71 mmol), Pd₂(dba)₃ (46 mg, 0.051 mmol), RuPhos (47 mg, 0.10 mmol), NaO^{t}Bu (127 mg, 1.32 mmol), and toluene (6.5 mL) was degassed and purged with N₂ 3 times, heated at 100° C. for 1 h, allowed to cool to rt, diluted (25 mL water), and then extracted (EtOAc). The combined organic layers were concentrated and purified by prep-TLC (50% EtOAc/petroleum ether) to give 3-(2-fluoro-3-(methoxymethoxy)-5-(trifluoromethyl)phenyl)-6-(4-(methylsulfonyl)piperazin-1-yl)-1-(tetrahydro-2H-pyran-2-yl)-1H-indazole (243 mg) as yellow solid. ¹H NMR (400 MHz, DMSO-d₆): δ 7.74 (d, 1H), 7.70-7.62 (m, 2H), 7.38 (d, 1H), 7.09-7.05 (m, 1H), 5.95-5.89 (m, 1H), 5.45 (s, 2H), 3.91 (d, 1H), 3.81-3.73 (m, 1H), 3.49 (s, 3H), 3.29-3.27 (m, 4H), 3.21 (d, 4H), 2.93 (s, 3H), 2.46-2.35 (m, 2H), 2.09-2.01 (m, 2H), 1.82-1.69 (m, 2H); LCMS: 587.3 [M+H]⁺.

### Step 2: 2-Fluoro-3-(6-(4-(methylsulfonyl)piperazin-1-yl)-1H-indazol-3-yl)-5-(trifluoromethyl)phenol

A mixture of 3-(2-fluoro-3-(methoxymethoxy)-5-(trifluoromethyl)phenyl)-6-(4-(methylsulfonyl)piperazin-1-yl)-1-(tetrahydro-2H-pyran-2-yl)-1H-indazole (200 mg), DCM (3.0 mL), and TFA (0.5 mL) was stirred at rt for 1.5 h under N₂, quenched by the slow addition of saturated NaHCO₃ (5 mL), diluted (10 mL water), and then extracted (EtOAc). The combined organic layers were concentrated and purified by prep-HPLC to give 2-fluoro-3-(6-(4-(methylsulfonyl)piperazin-1-yl)-1H-indazol-3-yl)-5-(trifluoromethyl)phenol (11 mg, 5% over 2 steps) as a white solid. ¹H NMR (400 MHz, DMSO-d₆): δ 12.53 (s, 1H), 6.71 (d, 1H), 6.57 (d, 1H), 6.48-6.35 (m, 2H), 6.25 (s, 1H), 2.48-2.42 (m, 4H), 2.38-2.35 (m, 4H), 2.11 (s, 3H); LCMS: 459.0 [M+H]⁺.

The Compounds below were synthesized in a similar manner to that described for Compound 11.

| Cmpd | Structure | Name | [M + H]⁺ |
|---|---|---|---|
| 11.01 | 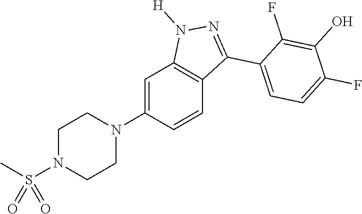 [SMILES: CS(=O)(=O)N1CCN(c2ccc3c(-c4ccc(F)c(O)c4F)n[nH]c3c2)CC1] | 2,6-Difluoro-3-(6-(4- (methylsulfonyl)piperazin-1-yl)- 1H-indazol-3-yl)phenol | 409.0 |
| 11.02 | 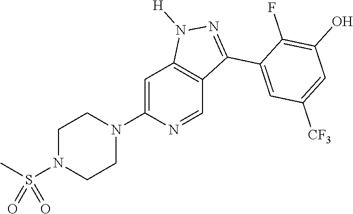 [SMILES: CS(=O)(=O)N1CCN(c2cc3[nH]nc(-c4cc(C(F)(F)F)cc(O)c4F)c3cn2)CC1] | 2-Fluoro-3-(6-(4- (methylsulfonyl)piperazin-1-yl)- 1H-pyrazolo[4,3-c]pyridin-3-yl)-5- (trifluoromethyl)phenol | 460.0 |
| 11.03 | 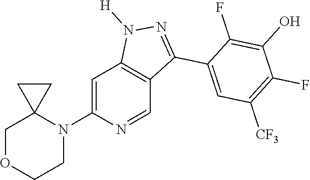 [SMILES: Oc1c(F)c(-c2n[nH]c3cc(N4CCOCC45CC5)ncc23)cc(C(F)(F)F)c1F] | 3-(6-(7-Oxa-4-azaspiro[2.5]octan- 4-yl)-1H-pyrazolo[4,3-c]pyridin-3- yl)-2,6-difluoro-5- (trifluoromethyl)phenol | 427.1 |
| 11.04 | 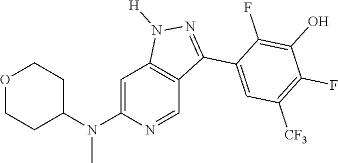 [SMILES: CN(c1cc2[nH]nc(-c3cc(C(F)(F)F)c(F)c(O)c3F)c2cn1)C1CCOCC1] | 2,6-Difluoro-3-(6- (methyl(tetrahydro-2H-pyran-4- yl)amino)-1H-pyrazolo[4,3- c]pyridin-3-yl)-5- (trifluoromethyl)phenol | 429.0 |

### Compound 12

### 2,6-Difluoro-3-(1-methyl-6-(4-(methylsulfonyl)piperazin-1-yl)-1H-pyrazolo[3,4-b]pyrazin-3-yl)-5-(trifluoromethyl)phenol

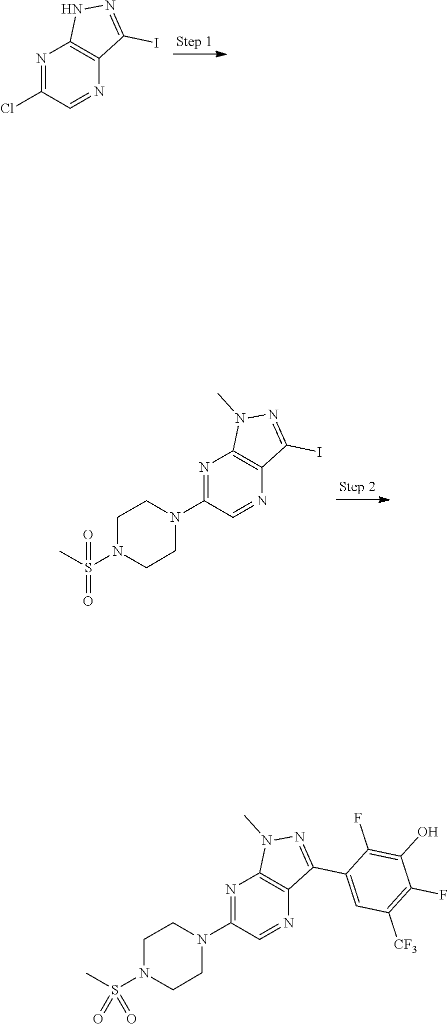 [SMILES: Clc1cnc2c(I)n[nH]c2n1 ; Cn1nc(I)c2ncc(N3CCN(S(C)(=O)=O)CC3)nc21 ; Cn1nc(-c2cc(C(F)(F)F)c(F)c(O)c2F)c2ncc(N3CCN(S(C)(=O)=O)CC3)nc21]

### Step 1: 3-Iodo-1-methyl-6-(4-(methylsulfonyl)piperazin-1-yl)-1H-pyrazolo[3,4-b]pyrazine

Iodomethane (67 uL, 1.07 mmol) was added to a solution of 6-chloro-3-iodopyrazolo[4,5-b]pyrazine (150 mg, 0.53 mmol) and K₂CO₃ (222 mg, 1.60 mmol) in THF (1 mL). The reaction was stirred at rt for 15 h, poured into water, and then extracted (DCM). The combined organics were concentrated and then taken up in DMA (4.0 mL). 1-Methanesulfonyl-piperazine (418 mg, 2.55 mmol) and DIEA (2.07 mL, 11.9 mmol) were added. The reaction was heated in a microwave at 100° C. for 1 h then partitioned between saturated NaHCO₃ and EtOAc. The aqueous layer was extracted (EtOAc). The combined organics were dried (Na₂SO₄), concentrated, and then purified by silica gel chromatography (0-100% EtOAc in hexanes) to give 1-(3-iodo-1-methylpyrazolo[4,5-e]pyrazin-6-yl)-4-(methylsulfonyl)piperazine (76 mg, 35%) as a white powder; LCMS: 422.9 [M+H]⁺ and 1-(3-iodo-2-methylpyrazolo[4,3-e]pyrazin-6-yl)-4-(methylsulfonyl)piperazine (43 mg, 20%) as a white powder; LCMS: 422.8 [M+H]⁺.

### Step 2: 2,6-Difluoro-3-(1-methyl-6-(4-(methylsulfonyl)piperazin-1-yl)-1H-pyrazolo[3,4-b]pyrazin-3-yl)-5-(trifluoromethyl)phenol

A mixture of 1-(3-iodo-1-methylpyrazolo[4,5-e]pyrazin-6-yl)-4-(methylsulfonyl)piperazine (50 mg, 0.12 mmol), Intermediate 11 (48 mg, 0.13 mmol), KF (28 mg, 0.47 mmol), and Pd(dppf)Cl₂ (8.7 mg, 0.012 mmol) in dioxane (2.0 mL) and water (0.50 mL) was purged with nitrogen for 5 min, heated in a microwave at 90° C. for 40 min, diluted with water, and then extracted with EtOAc. The combined organics were dried (Na₂SO₄) and then concentrated. The residue was taken up in TFA (1.0 mL) and DCM (1.0 mL), stirred at rt for 15 h, concentrated, and then purified by prep-HPLC to give 2,6-difluoro-3-(1-methyl-6-(4-(methylsulfonyl)piperazin-1-yl)-1H-pyrazolo[3,4-b]pyrazin-3-yl)-5-(trifluoromethyl)phenol (21 mg, 36%) as a white powder. ¹H NMR (400 MHz, DMSO-d₆): δ 11.23 (s, 1H), 8.58 (s, 1H), 8.02 (t, J=7.1 Hz, 1H), 3.97 (s, 3H), 3.92 (br s, 4H), 3.28 (br d, J=4.4 Hz, 4H), 2.92 (s, 3H). LCMS: 493.0 [M+H]⁺.

The Compounds below were synthesized in a similar manner to that described for Compound 12.

| Cmpd | Structure | Name | [M + H]⁺ |
|---|---|---|---|
| 12.01 | 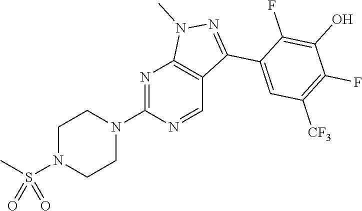 [SMILES: Cn1nc(-c2cc(C(F)(F)F)c(F)c(O)c2F)c2cnc(N3CCN(S(C)(=O)=O)CC3)nc21] | 2,6-Difluoro-3-(1-methyl-6-(4- (methylsulfonyl)piperazin-1-yl)- 1H-pyrazolo[3,4-d]pyrimidin-3-yl)- 5-(trifluoromethyl)phenol | 493.0 |
| 12.02 | 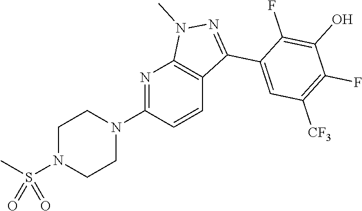 [SMILES: Cn1nc(-c2cc(C(F)(F)F)c(F)c(O)c2F)c2ccc(N3CCN(S(C)(=O)=O)CC3)nc21] | 2,6-Difluoro-3-(1-methyl-6-(4- (methylsulfonyl)piperazin-1-yl)- 1H-pyrazolo[3,4-b]pyridin-3-yl)-5- (trifluoromethyl)phenol | 492.0 |
| 12.03 | 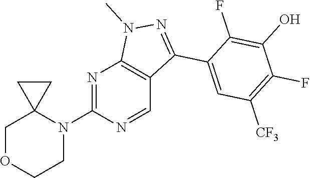 [SMILES: Cn1nc(-c2cc(C(F)(F)F)c(F)c(O)c2F)c2cnc(N3CCOCC34CC4)nc21] | 2,6-Difluoro-3-(1-methyl-6-(7-oxa- 4-azaspiro[2.5]octan-4-yl)-1H- pyrazolo[3,4-d]pyrimidin-3-yl)-5- (trifluoromethyl)phenol | 442.1 |
| 12.04 | 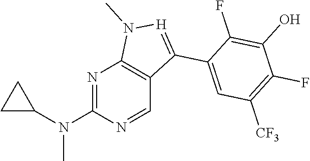 | 3-(6-(Cyclopropyl(methyl)amino)- 1-methyl-1H-pyrazolo[3,4- d]pyrimidin-3-yl)-2,6-difluoro-5- (trifluoromethyl)phenol | 400.0 |
| 12.05 | 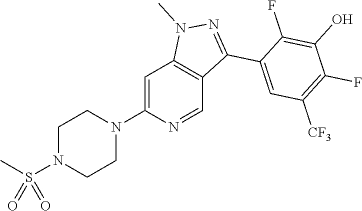 [SMILES: Cn1nc(-c2cc(C(F)(F)F)c(F)c(O)c2F)c2cnc(N3CCN(S(C)(=O)=O)CC3)cc21] | 2,6-Difluoro-3-(1-methyl-6-(4- (methylsulfonyl)piperazin-1-yl)- 1H-pyrazolo[4,3-c]pyridin-3-yl)-5- (trifluoromethyl)phenol | 492.1 |
| 12.06 (1,5) | 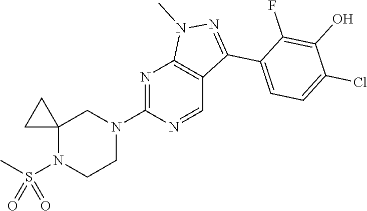 [SMILES: Cn1nc(-c2ccc(Cl)c(O)c2F)c2cnc(N3CCN(S(C)(=O)=O)C4(CC4)C3)nc21] | 6-Chloro-2-fluoro-3-(1-methyl-6- (4-(methylsulfonyl)-4,7- diazaspiro[2.5]octan-7-yl)-1H- pyrazolo[3,4-d]pyrimidin-3- yl)phenol | 467.3 |
| 12.07 (1,2) | 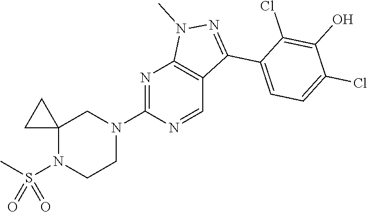 [SMILES: Cn1nc(-c2ccc(Cl)c(O)c2Cl)c2cnc(N3CCN(S(C)(=O)=O)C4(CC4)C3)nc21] | 2,6-Dichloro-3-(1-methyl-6-(4- (methylsulfonyl)-4,7- diazaspiro[2.5]octan-7-yl)-1H- pyrazolo[3,4-d]pyrimidin-3- yl)phenol | 483.3 |
| 12.08 (3,4) | 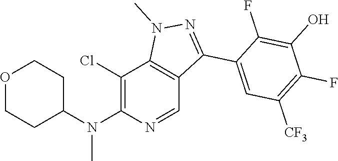 [SMILES: CN(c1ncc2c(-c3cc(C(F)(F)F)c(F)c(O)c3F)nn(C)c2c1Cl)C1CCOCC1] | 3-(7-Chloro-1-methyl-6- (methyl(tetrahydro-2H-pyran-4- yl)amino)-1H-pyrazolo[4,3- c]pyridin-3-yl)-2,6-difluoro-5- (trifluoromethyl)phenol | 477.0 |
| 12.09 (1,5) | 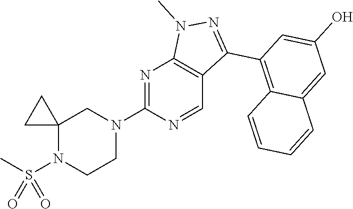 [SMILES: Cn1nc(-c2cc(O)cc3ccccc23)c2cnc(N3CCN(S(C)(=O)=O)C4(CC4)C3)nc21] | 4-(1-Methyl-6-(4-(methylsulfonyl)- 4,7-diazaspiro[2.5]octan-7-yl)-1H- pyrazolo[3,4-d]pyrimidin-3- yl)naphthalen-2-ol | 465.4 |
| 12.10 (1,5) | 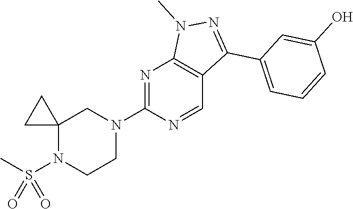 [SMILES: Cn1nc(-c2cccc(O)c2)c2cnc(N3CCN(S(C)(=O)=O)C4(CC4)C3)nc21] | 3-(1-Methyl-6-(4-(methylsulfonyl)- 4,7-diazaspiro[2.5]octan-7-yl)-1H- pyrazolo[3,4-d]pyrimidin-3- yl)phenol | 415.3 |

Alternate conditions:
1. Step 1: 120° C., 90 min.
2. Step 2: Used (3-(benzyloxy)-2,4-dichlorophenyl)boronic acid and then debenzylated (5 wt. % palladium on carbon, palladium hydroxide on carbon, THF, H₂, rt, 15 h).
3. Step 2 only from Intermediate 22.
4. Step 2: Pd(dppf)Cl₂ .CH₂Cl₂, 2 M Na₂CO₃, dioxane, 80° C., 2 h then TFA, 70° C., 2 h;
5. Unprotected phenol was used.

### Compound 13

### 2-Fluoro-3-(6-(1-(methylsulfonyl)piperidin-4-yl)-1H-pyrazolo[4,3-c]pyridin-3-yl)-5-(trifluoromethyl)phenol

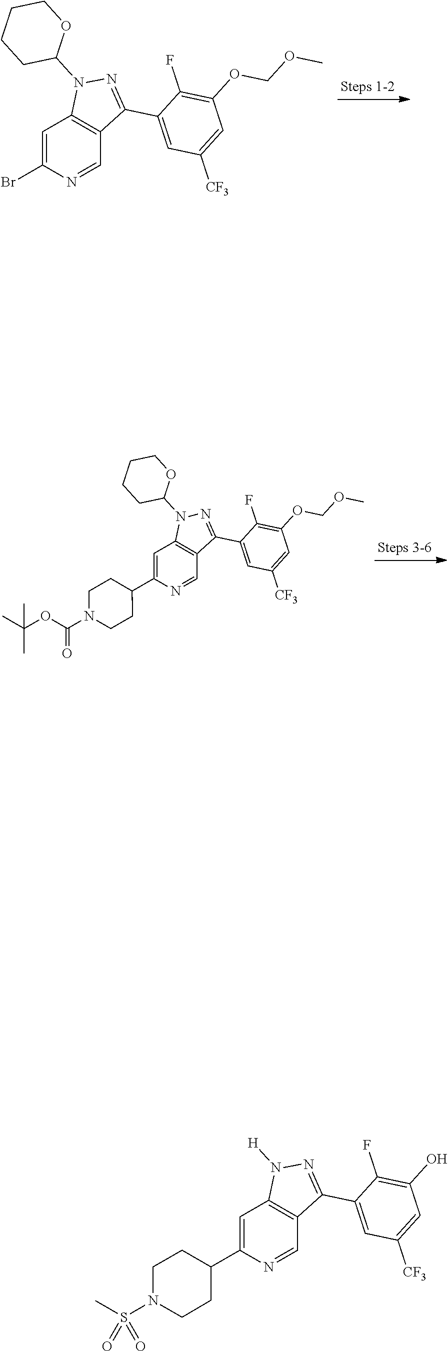

### Step 1: tert-Butyl 4-(3-(2-fluoro-3-(methoxymethoxy)-5-(trifluoromethyl)phenyl)-1-(tetrahydro-2H-pyran-2-yl)-1H-pyrazolo[4,3-c]pyridin-6-yl)-5,6-dihydropyridine-1(2H)-carboxylate

Pd(dppf)Cl₂·CH₂Cl₂ (39 mg, 0.05 mmol) was added to a mixture of Intermediate 18.09 (480 mg, 0.95 mmol), tert-butyl 4-(4,4,5,5-tetramethyl-1,3,2-dioxaborolan-2-yl)-5,6-dihydropyridine-1(2H)-carboxylate (353 mg, 1.14 mmol), K₃PO₄ (1.01 g, 4.76 mmol), THF (10 mL), and H₂O (4 mL). The mixture was degassed and purged with N₂ 3 times, heated at 80° C. for 4 h, allowed to cool to rt, poured into water (30 mL), and then extracted (3×30 mL EtOAc). The combined organic layers were washed (2×30 mL brine), dried (Na₂SO₄), filtered, and then concentrated. The residue was purified by silica gel chromatography (petroleum ether/EtOAc=80/1 to 40/1) to give tert-butyl 4-(3-(2-fluoro-3-(methoxymethoxy)-5-(trifluoromethyl)phenyl)-1-(tetrahydro-2H-pyran-2-yl)-1H-pyrazolo[4,3-c]pyridin-6-yl)-5,6-dihydropyridine-1(2H)-carboxylate (490 mg, 84%) as a yellow solid. ¹H NMR (400 MHz, DMSO-d₆): δ 9.07 (d, 1H), 7.87 (s, 1H), 7.79-7.68 (m, 2H), 6.91 (s, 1H), 6.07 (d, 1H), 5.47 (s, 2H), 4.10 (s, 2H), 3.93 (s, 2H), 3.86-3.78 (m, 1H), 3.60 (t, 2H), 3.49 (s, 3H), 2.66 (s, 2H), 2.04 (s, 2H), 1.84-1.69 (m, 1H), 1.62 (d, 2H), 1.44 (s, 9H); LCMS: 607.3 [M+H]⁺.

### Step 2: tert-Butyl 4-(3-(2-fluoro-3-(methoxymethoxy)-5-(trifluoromethyl)phenyl)-1-(tetrahydro-2H-pyran-2-yl)-1H-pyrazolo[4,3-c]pyridin-6-yl)piperidine-1-carboxylate

Palladium on carbon (480 mg, 0.79 mmol, 10% wt) was added to a solution of tert-butyl 4-(3-(2-fluoro-3-(methoxymethoxy)-5-(trifluoromethyl)phenyl)-1-(tetrahydro-2H-pyran-2-yl)-1H-pyrazolo[4,3-c]pyridin-6-yl)-5,6-dihydropyridine-1(2H)-carboxylate (480 mg, 0.79 mmol) in MeOH (10 mL). The mixture was stirred at rt for 2 h under a balloon of hydrogen, filtered, and then concentrated to give tert-butyl 4-(3-(2-fluoro-3-(methoxymethoxy)-5-(trifluoromethyl)phenyl)-1-(tetrahydro-2H-pyran-2-yl)-1H-pyrazolo[4,3-c]pyridin-6-yl)piperidine-1-carboxylate (450 mg) as a yellow solid. LCMS: 609.3 [M+H]⁺.

### Step 3: 2-Fluoro-3-(6-(piperidin-4-yl)-1-(tetrahydro-2H-pyran-2-yl)-1H-pyrazolo[4,3-c]pyridin-3-yl)-5-(trifluoromethyl)phenol

Trifluoroacetic acid (1 mL, 13.5 mmol) was added to a solution of tert-butyl 4-(3-(2-fluoro-3-(methoxymethoxy)-5-(trifluoromethyl)phenyl)-1-(tetrahydro-2H-pyran-2-yl)-1H-pyrazolo[4,3-c]pyridin-6-yl)piperidine-1-carboxylate (340 mg, 0.55 mmol) in DCM (7 mL). The mixture was stirred at rt for 2 h and concentrated to give 2-fluoro-3-(6-(piperidin-4-yl)-1-(tetrahydro-2H-pyran-2-yl)-1H-pyrazolo[4,3-c]pyridin-3-yl)-5-(trifluoromethyl)phenol (320 mg) as a yellow oil. LCMS: 465.3 [M+H]⁺.

### Step 4: 2-Fluoro-3-(6-(1-(methylsulfonyl)piperidin-4-yl)-1-(tetrahydro-2H-pyran-2-yl)-1H-pyrazolo[4,3-c]pyridin-3-yl)-5-(trifluoromethyl)phenyl methanesulfonate

Triethylamine (560 mg, 5.53 mmol) and MsCl (317 mg, 2.77 mmol) were added to a solution of 2-fluoro-3-(6-(piperidin-4-yl)-1-(tetrahydro-2H-pyran-2-yl)-1H-pyrazolo[4,3-c]pyridin-3-yl)-5-(trifluoromethyl)phenol (320 mg) in DCM (8 mL) at 0° C. The mixture was stirred at rt for 2 h, poured into water (30 mL), and then extracted with EtOAc (3×35 mL). The combined organic layers were washed (2×30 mL brine), dried (Na₂SO₄), filtered, and then concentrated. The residue was purified by silica gel chromatography (petroleum ether/EtOAc=80/1 to 50/1) to give 2-fluoro-3-(6-(1-(methylsulfonyl)piperidin-4-yl)-1-(tetrahydro-2H-pyran-2-yl)-1H-pyrazolo[4,3-c]pyridin-3-yl)-5-(trifluoromethyl)phenyl methanesulfonate (240 mg, 69%) as a yellow solid. LCMS: 621.2 [M+H]⁺.

### Step 5: 2-Fluoro-3-(6-(1-(methylsulfonyl)piperidin-4-yl)-1H-pyrazolo[4,3-c]pyridin-3-yl)-5-(trifluoromethyl)phenyl methanesulfonate

A solution of 2-fluoro-3-(6-(1-(methylsulfonyl)piperidin-4-yl)-1-(tetrahydro-2H-pyran-2-yl)-1H-pyrazolo[4,3-c]pyridin-3-yl)-5-(trifluoromethyl)phenyl methanesulfonate (240 mg, 0.38 mmol) in HCl/EtOAc (5 mL, 4M) was stirred at rt for 2 h and then concentrated to give 2-fluoro-3-(6-(1-(methylsulfonyl)piperidin-4-yl)-1H-pyrazolo[4,3-c]pyridin-3-yl)-5-(trifluoromethyl)phenyl methanesulfonate (220 mg) as a yellow solid. LCMS: 537.1 [M+H]⁺.

### Step 6: 2-Fluoro-3-(6-(1-(methylsulfonyl)piperidin-4-yl)-1H-pyrazolo[4,3-c]pyridin-3-yl)-5-(trifluoromethyl)phenol

LiOH·H₂O (161 mg, 3.84 mmol) was added to a mixture of 2-fluoro-3-(6-(1-(methylsulfonyl)piperidin-4-yl)-1H-pyrazolo[4,3-c]pyridin-3-yl)-5-(trifluoromethyl)phenyl methanesulfonate (220 mg, 0.38 mmol) in THE (4 mL), H₂O (2 mL), and MeOH (1 mL). The mixture was stirred at rt for 2 h. 1 M HCl (˜5 mL) was added to adjust the pH to ˜7, and the mixture was extracted (3×3 5 mL EtOAc). The combined organic layers were washed (2×30 mL brine), dried (Na₂SO₄), filtered, and then concentrated. The residue was purified by prep-HPLC to give 2-fluoro-3-(6-(1-(methylsulfonyl)piperidin-4-yl)-1H-pyrazolo[4,3-c]pyridin-3-yl)-5-(trifluoromethyl)phenol (63 mg, 35%) as a white solid. ¹H NMR (400 MHz, DMSO-d₆): δ 14.89 (s, 1H), 11.18 (s, 1H), 9.43 (s, 1H), 7.95 (s, 1H), 7.61 (d, 1H), 7.52-7.42 (m, 1H), 3.77 (d, 2H), 3.24 (s, 1H), 2.95 (s, 3H), 2.88 (t, 2H), 2.12 (d, 2H), 2.04-1.86 (m, 2H); LCMS: 459.0 [M+H]⁺.

The Compound below was synthesized in a similar manner to that described for Compound 13.

| Cmpd | Structure | Name | [M + H]⁺ |
|---|---|---|---|
| 13.01 | 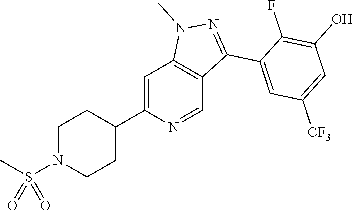 [SMILES: Cn1nc(-c2cc(C(F)(F)F)cc(O)c2F)c2cnc(C3CCN(S(C)(=O)=O)CC3)cc21] | 2-Fluoro-3-(1-methyl-6-(1- (methylsulfonyl)piperidin-4-y1)- 1H-pyrazolo[4,3-c]pyridin-3-y1)-5- (trifluoromethyl)phenol | 473.0 |

### Compound 14

### 2-Fluoro-5-(6-(1-(methylsulfonyl)-1,2,3,6-tetrahydropyridin-4-yl)-1H-indazol-3-yl)-3-(trifluoromethyl)phenol

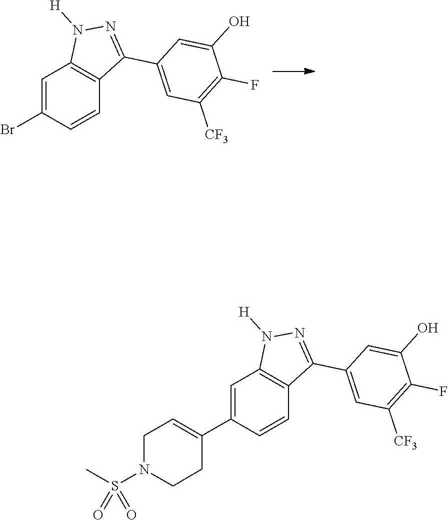 [SMILES: Oc1cc(-c2n[nH]c3cc(Br)ccc23)cc(C(F)(F)F)c1F ; CS(=O)(=O)N1CC=C(c2ccc3c(-c4cc(O)c(F)c(C(F)(F)F)c4)n[nH]c3c2)CC1]

A mixture of Intermediate 20 (0.18 g, 0.48 mmol), 1-methanesulfonyl-4-(tetramethyl-1,3,2-dioxaborolan-2-yl)-1,2,3,6-tetrahydropyridine (0.17 g, 0.58 mmol), K₂CO₃ (0.27 g, 1.92 mmol), and Pd(dppf)Cl₂ (35 mg, 0.048 mmol) in dioxane (4.0 mL) and water (4.0 mL) was purged with nitrogen for 5 min, heated in a microwave at 90° C. for 30 min, diluted with water, and then extracted with EtOAc. The combined organics were dried (Na₂SO₄), concentrated, and then purified by silica gel chromatography (0-100% EtOAc/hexanes) to give 2-fluoro-5-(6-(1-(methylsulfonyl)-1,2,3,6-tetrahydropyridin-4-yl)-1H-indazol-3-yl)-3-(trifluoromethyl)phenol (0.17 g, 78%) as a white powder. ¹H NMR (400 MHz, DMSO-d₆): 13.41 (s, 1H), 10.85 (s, 1H), 7.93 (dd, J=8.3, 14.2 Hz, 2H), 7.64 (br d, J=5.0 Hz, 1H), 7.57 (s, 1H), 7.46 (d, J=8.7 Hz, 1H), 6.36 (br s, 1H), 3.92 (br s, 2H), 3.43 (t, J=5.6 Hz, 2H), 2.97 (s, 3H), 2.72 (br s, 2H); LCMS: 456.1 [M+H]⁺.

The Compounds below were synthesized in a similar manner to that described for Compound 14.

| Cmpd | Structure | Name | [M + H]⁺ |
|---|---|---|---|
| 14.01 | 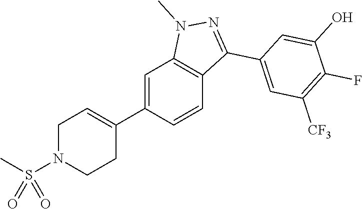 [SMILES: Cn1nc(-c2cc(O)c(F)c(C(F)(F)F)c2)c2ccc(C3=CCN(S(C)(=O)=O)CC3)cc21] | 2-Fluoro-5-(1-methyl-6-(1- (methylsulfonyl)-1,2,3,6- tetrahydropyridin-4-y1)-1H-indazol- 3-y1)-3-(trifluoromethyl)phenol | 470.3 |
| 14.02 | 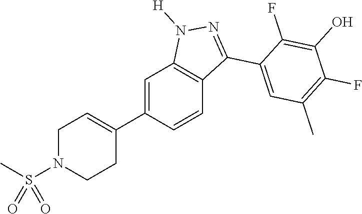 [SMILES: Cc1cc(-c2n[nH]c3cc(C4=CCN(S(C)(=O)=O)CC4)ccc23)c(F)c(O)c1F] | 2,6-Difluoro-3-methyl-5-(6-(1- (methylsulfonyl)-1,2,3,6- tetrahydropyridin-4-y1)-1H-indazol- 3-y1)phenol | 420.1 |
| 14.03 | 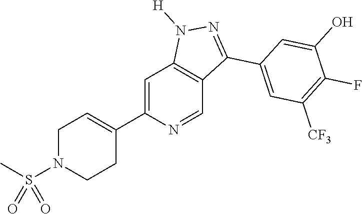 [SMILES: CS(=O)(=O)N1CC=C(c2cc3[nH]nc(-c4cc(O)c(F)c(C(F)(F)F)c4)c3cn2)CC1] | 2-Fluoro-5-(6-(1-(methylsulfony1)- 1,2,3,6-tetrahydropyridin-4-yl)-1H- pyrazolo[4,3-c]pyridin-3-yl)-3- (trifluoromethyl)phenol | 457.0 |

### Compound 15

### 2-Fluoro-5-(6-(1-(methylsulfonyl)piperidin-4-yl)-1H-indazol-3-yl)-3-(trifluoromethyl)phenol

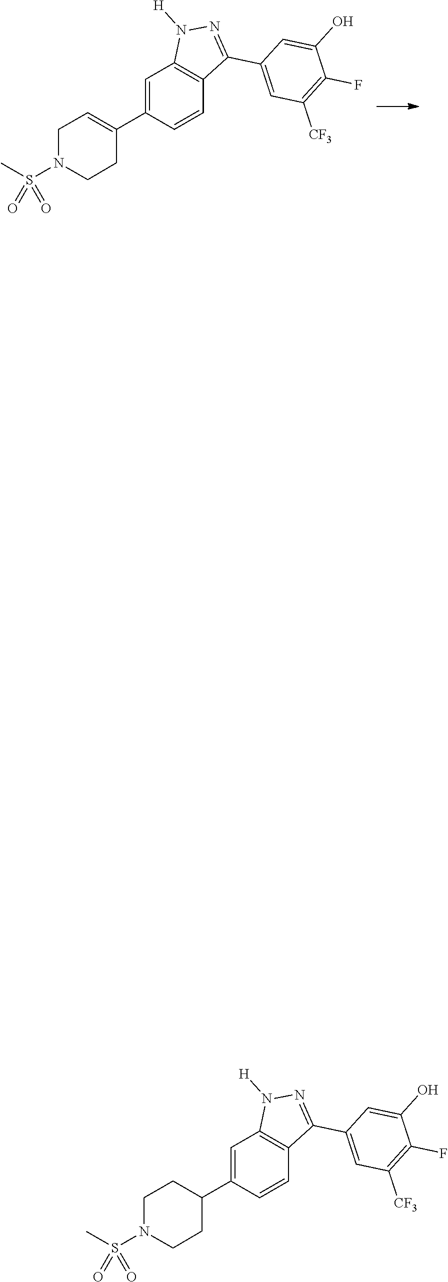 [SMILES: CS(=O)(=O)N1CC=C(c2ccc3c(-c4cc(O)c(F)c(C(F)(F)F)c4)n[nH]c3c2)CC1 ; CS(=O)(=O)N1CCC(c2ccc3c(-c4cc(O)c(F)c(C(F)(F)F)c4)n[nH]c3c2)CC1]

A mixture of Compound 14 (100 mg, 0.22 mmol), palladium hydroxide on carbon (20 wt %, 31 mg, 0.04 mmol), and methanol (10 mL) was evacuated/purged with nitrogen 3 times, stirred under a balloon of hydrogen for 2 h, filtered, concentrated, and then purified by silica gel chromatography (0-100% EtOAc/hexanes) to give 2-fluoro-5-(6-(1-(methylsulfonyl)piperidin-4-yl)-1H-indazol-3-yl)-3-(trifluoromethyl)phenol (874 mg, 87%) as a white powder. ¹H NMR (400 MHz, DMSO-d₆): δ 13.31 (s, 1H), 10.83 (s, 1H), 7.91 (t, J=7.8 Hz, 2H), 7.63 (br d, J=5.1 Hz, 1H), 7.44 (s, 1H), 7.23 (d, J=8.4 Hz, 1H), 3.72 (br d, J=11.7 Hz, 2H), 2.93 (s, 3H), 2.90-2.78 (m, 3H), 2.02-1.93 (m, 2H), 1.87-1.69 (m, 2H); LCMS: 458.2 [M+H]⁺.

The Compounds below were synthesized in a similar manner to that described for Compound 15.

| Cmpd | Structure | Name | [M + H]⁺ |
|---|---|---|---|
| 15.01 | 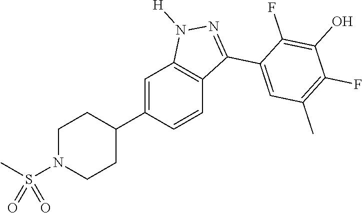 [SMILES: Cc1cc(-c2n[nH]c3cc(C4CCN(S(C)(=O)=O)CC4)ccc23)c(F)c(O)c1F] | 2,6-Difluoro-3-methyl-5-(6-(1- (methylsulfony1)piperidin-4-y1)- 1H-indazol-3-yl)phenol | 422.1 |
| 15.02 | 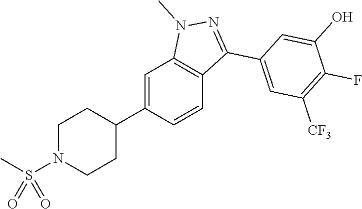 [SMILES: Cn1nc(-c2cc(O)c(F)c(C(F)(F)F)c2)c2ccc(C3CCN(S(C)(=O)=O)CC3)cc21] | 2-Fluoro-5-(1-methyl-6-(1- (methylsulfonyl)piperidin-4-y1)- 1H-indazol-3-yl)-3- (trifluoromethyl)phenol | 472.0 |
| 15.03 | 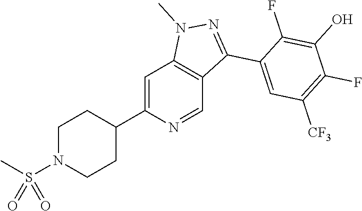 [SMILES: Cn1nc(-c2cc(C(F)(F)F)c(F)c(O)c2F)c2cnc(C3CCN(S(C)(=O)=O)CC3)cc21] | 2,6-Difluoro-3-(1-methyl-6-(1- (methylsulfony1)piperidin-4-y1)- 1H-pyrazolo[4,3-c]pyridin-3-y1)-5- (trifluoromethyl)phenol | 491.1 |
| 15.04 | 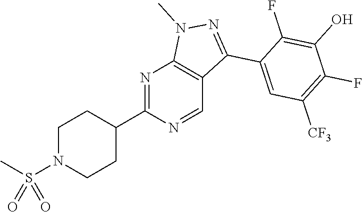 [SMILES: Cn1nc(-c2cc(C(F)(F)F)c(F)c(O)c2F)c2cnc(C3CCN(S(C)(=O)=O)CC3)nc21] | 2,6-Difluoro-3-(1-methyl-6-(1- (methylsulfony1)piperidin-4-y1)- 1H-pyrazolo[3,4-d]pyrimidin-3-yl)- 5-(trifluoromethyl)phenol | 492.1 |

### Compound 16

### 2-Chloro-4-(3-(4-fluoro-3-hydroxyphenyl)-1H-indazol-6-yl)phenol

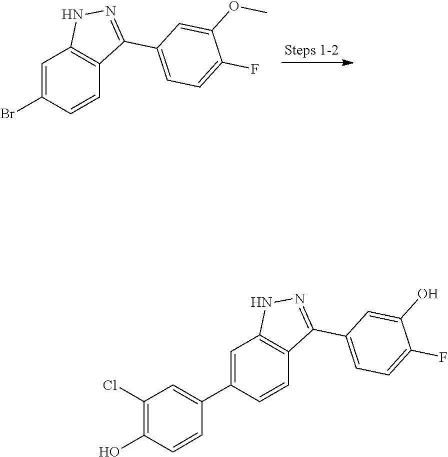 [SMILES: COc1cc(-c2n[nH]c3cc(Br)ccc23)ccc1F ; Oc1cc(-c2n[nH]c3cc(-c4ccc(O)c(Cl)c4)ccc23)ccc1F]

### Step 1: 6-(3-Chloro-4-methoxyphenyl)-3-(4-fluoro-3-methoxyphenyl)-1H-indazole

A mixture of Intermediate 18.06 (1.5 g, 4.67 mmol), 3-chloro-4-methoxyphenylboronic acid (871 mg, 4.67 mmol), Pd(dppf)Cl₂ (342 mg, 0.47 mmol), Na₂CO₃ (1.49 g, 14.0 mmol), dioxane (45 mL), and H₂O (10 mL) was degassed with 3 vacuum/N₂ cycles, stirred at 100° C. overnight, allowed to cool to rt, slowly poured into H₂O (30 mL), and then extracted (3×40 mL EtOAc). The combined organic layers were washed (100 mL brine), dried (Na₂SO₄), filtered, and then concentrated. The residue was purified by silica gel chromatography (10% EtOAc/petroleum ether) to give 6-(3-chloro-4-methoxyphenyl)-3-(4-fluoro-3-methoxyphenyl)-1H-indazole (600 mg, 16%) as a white solid. ¹H NMR (400 MHz, DMSO-d₆): δ 13.32 (s, 1H), 8.11 (d, 1H), 7.83 (d, 1H), 7.77 (s, 1H), 7.75-7.66 (m, 2H), 7.58-7.55 (m, 1H), 7.50 (d, 1H), 7.36 (d, 1H), 7.27 (d, 1H), 3.97 (s, 3H), 3.92 (s, 3H); LCMS: 383.1 [M+H]⁺.

### Step 2: 2-Chloro-4-(3-(4-fluoro-3-hydroxyphenyl)-1H-indazol-6-yl)phenol

Boron tribromide (250 μL, 2.61 mmol) was added to a mixture of 6-(3-chloro-4-methoxyphenyl)-3-(4-fluoro-3-methoxyphenyl)-1H-indazole (200 mg, 0.52 mmol) in DCM (5 mL) at −78° C. The mixture was stirred at rt for 2 h, slowly poured into MeOH (5 mL), and then stirred for 0.5 h. Saturated NaHCO₃ (30 mL) was added, and the mixture was extracted (3×20 mL EtOAc). The combined organic layers were washed (20 mL brine), dried (Na₂SO₄), filtered, and then concentrated. The residue was purified by prep-HPLC [water (0.04% HCl)/CH₃CN] to give 2-chloro-4-(3-(4-fluoro-3-hydroxyphenyl)-1H-indazol-6-yl)phenol (56 mg, 29%) as a white solid. ¹H NMR (400 MHz, DMSO-d₆): δ 13.23 (s, 1H), 10.73-9.61 (m, 2H), 8.02 (d, 1H), 7.68-7.75 (m, 2H), 7.64 (d, 1H), 7.56 (d, 1H), 7.50-7.38 (m, 2H), 7.26 (d, 1H), 7.10 (d, 1H); LCMS: 355.1 [M+H]⁺.

### Compound 17

### 2-Chloro-4-(3-(4-fluoro-3-hydroxyphenyl)-1-methyl-1H-indazol-6-yl)phenol

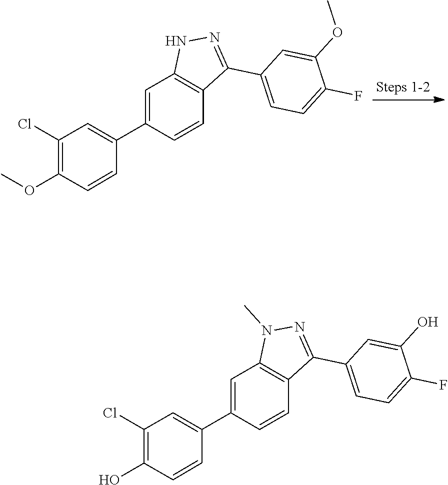 [SMILES: COc1cc(-c2n[nH]c3cc(-c4ccc(OC)c(Cl)c4)ccc23)ccc1F ; Cn1nc(-c2ccc(F)c(O)c2)c2ccc(-c3ccc(O)c(Cl)c3)cc21]

### Step 1: 6-(3-Chloro-4-methoxyphenyl)-3-(4-fluoro-3-methoxyphenyl)-1-methyl-1H-indazole

Sodium hydride (25 mg, 0.627 mmol, 60% purity) was slowly added to a solution of Compound 16, Step 1 (200 mg, 0.52 mmol) in DMF (3 mL) at 0° C. After stirring for 1 h, iodomethane (85 mg, 0.6 mmol) was added to the mixture. The reaction was warmed to rt, stirred for 1 h, poured into saturated NH₄Cl (100 mL), and then extracted (3×50 mL EtOAc). The combined organic layers were washed (50 mL brine), dried (Na₂SO₄), filtered, and then concentrated. The residue was purified by silica gel chromatography (60% EtOAc/petroleum ether) to give 6-(3-chloro-4-methoxyphenyl)-3-(4-fluoro-3-methoxyphenyl)-1-methyl-1H-indazole (150 mg, 72%) as a white solid. ¹H NMR (400 MHz, DMSO-d₆): δ 8.10 (d, 1H), 8.00 (s, 1H), 7.93 (d, 1H), 7.79 (d, 1H), 7.65 (d, 1H), 7.58-7.50 (m, 2H), 7.31-7.30 (m, 1H), 7.28 (d, 1H), 4.17 (s, 3H), 3.96 (s, 3H), 3.93 (s, 3H); LCMS: 397.2 [M+H]⁺.

### Step 2: 2-Chloro-4-(3-(4-fluoro-3-hydroxyphenyl)-1-methyl-1H-indazol-6-yl)phenol

2-Chloro-4-(3-(4-fluoro-3-hydroxyphenyl)-1-methyl-1H-indazol-6-yl)phenol was synthesized from 6-(3-chloro-4-methoxyphenyl)-3-(4-fluoro-3-methoxyphenyl)-1-methyl-1H-indazole following the procedure described for Compound 16, Step 2. ¹H NMR (400 MHz, DMSO-d₆): δ 10.17 (s, 2H), 8.01 (d, 1H), 7.93 (s, 1H), 7.83 (d, 1H), 7.70-7.57 (m, 2H), 7.51 (d, 1H), 7.44-7.36 (m, 1H), 7.28-7.23 (m, 1H), 7.09 (d, 1H), 4.13 (s, 3H); LCMS: 367.0 [M−H]⁻.

### Compound 18

### 2,6-Difluoro-3-(1-methyl-6-(5-oxa-8-azaspiro[3.5]nonan-8-yl)-1H-pyrazolo[3,4-d]pyrimidin-3-yl)-5-(trifluoromethyl)phenol

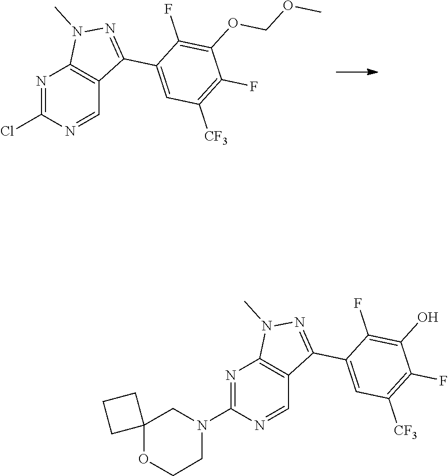 [SMILES: COCOc1c(F)c(-c2nn(C)c3nc(Cl)ncc23)cc(C(F)(F)F)c1F ; Cn1nc(-c2cc(C(F)(F)F)c(F)c(O)c2F)c2cnc(N3CCOC4(CCC4)C3)nc21]

A mixture of Intermediate 18.04 (100 mg, 0.24 mmol), 5-oxa-8-azaspiro[3.5]nonane (62 mg, 0.49 mmol), Hunig's base (0.43 mL, 2.45 mmol), and DMA (5 mL) was heated at 100° C. for 1 h in a microwave and then partitioned between saturated ammonium chloride and EtOAc. The aqueous layer was extracted with EtOAc. The combined organics were dried (Na₂SO₄) and then concentrated (note: aqueous work up was omitted in some of the other examples). The residue was purified by RP-HPLC to yield 2,6-difluoro-3-(1-methyl-6-(5-oxa-8-azaspiro[3.5]nonan-8-yl)-1H-pyrazolo[3,4-d]pyrimidin-3-yl)-5-(trifluoromethyl)phenol as a white powder (45 mg, 40%). ¹H NMR (400 MHz, DMSO-d₆): δ 11.67-11.11 (m, 1H), 8.94 (d, J=3.3 Hz, 1H), 7.57 (t, J=7.2 Hz, 1H), 3.92 (s, 3H), 3.87 (s, 2H), 3.84-3.78 (m, 2H), 3.62 (t, J=4.8 Hz, 2H), 2.01-1.91 (m, 4H), 1.85-1.62 (m, 2H); LCMS: 456.2 [M-+H]⁺.

The Compounds below were synthesized in a similar manner to that described for Compound 18.

| Cmpd | Structure | Name | [M + H]⁺ |
|---|---|---|---|
| 18.01 (4) | 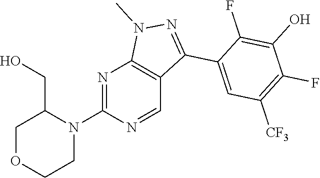 [SMILES: Cn1nc(-c2cc(C(F)(F)F)c(F)c(O)c2F)c2cnc(N3CCOCC3CO)nc21] | 2,6-Difluoro-3-(6-(3- (hydroxymethyl)morpholino)-1- methyl-1H-pyrazolo[3,4- d]pyrimidin-3-y1)-5- (trifluoromethyl)phenol | 446.6 |
| 18.02 | 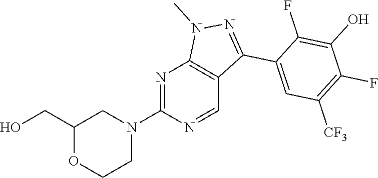 [SMILES: Cn1nc(-c2cc(C(F)(F)F)c(F)c(O)c2F)c2cnc(N3CCOC(CO)C3)nc21] | 2,6-Difluoro-3-(6-(2- (hydroxymethyl)morpholino)-1- methyl-1H-pyrazolo[3,4- d]pyrimidin-3-y1)-5- (trifluoromethyl)phenol | 446.3 |
| 18.03 (4) | 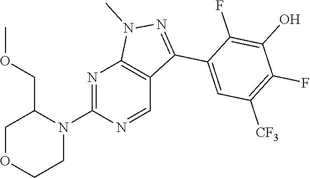 [SMILES: COCC1COCCN1c1ncc2c(-c3cc(C(F)(F)F)c(F)c(O)c3F)nn(C)c2n1] | 2,6-Difluoro-3-(6-(3- (methoxymethyl)morpholino)-1- methyl-1H-pyrazolo[3,4- d]pyrimidin-3-y1)-5- (trifluoromethyl)phenol | 460.5 |
| 18.04 | 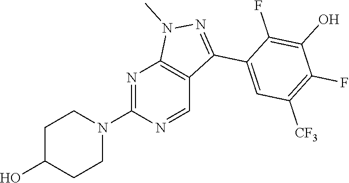 [SMILES: Cn1nc(-c2cc(C(F)(F)F)c(F)c(O)c2F)c2cnc(N3CCC(O)CC3)nc21] | 1-(3-(2,4-Difluoro-3-hydroxy-5- (trifluoromethyl)phenyl)-1-methyl- 1H-pyrazolo[3,4-d]pyrimidin-6- y1)piperidin-4-ol | 430.5 |
| 18.05 | 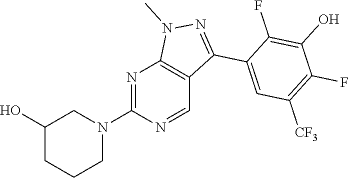 [SMILES: Cn1nc(-c2cc(C(F)(F)F)c(F)c(O)c2F)c2cnc(N3CCCC(O)C3)nc21] | 1-(3-(2,4-Difluoro-3-hydroxy-5- (trifluoromethyl)phenyl)-1-methyl- 1H-pyrazolo[3,4-d]pyrimidin-6- y1)piperidin-3-ol | 430.5 |
| 18.06 | 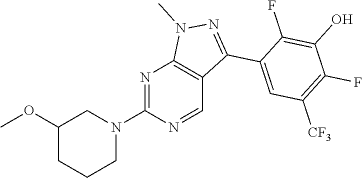 [SMILES: COC1CCCN(c2ncc3c(-c4cc(C(F)(F)F)c(F)c(O)c4F)nn(C)c3n2)C1] | 2,6-Difluoro-3-(6-(3- methoxypiperidin-1-yl)-1-methyl- 1H-pyrazolo[3,4-d]pyrimidin-3-yl)- 5-(trifluoromethyl)phenol | 444.6 |
| 18.07 | 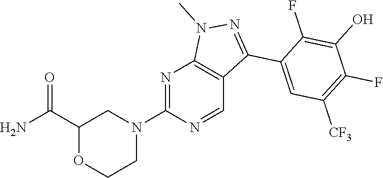 [SMILES: Cn1nc(-c2cc(C(F)(F)F)c(F)c(O)c2F)c2cnc(N3CCOC(C(N)=O)C3)nc21] | 4-(3-(2,4-Difluoro-3-hydroxy-5- (trifluoromethyl)phenyl)-1-methyl- 1H-pyrazolo[3,4-d]pyrimidin-6- yl)morpholine-2-carboxamide | 459.4 |
| 18.08 | 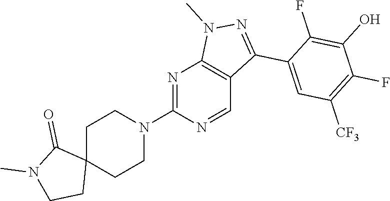 [SMILES: CN1CCC2(CCN(c3ncc4c(-c5cc(C(F)(F)F)c(F)c(O)c5F)nn(C)c4n3)CC2)C1=O] | 8-(3-(2,4-Difluoro-3-hydroxy-5- (trifluoromethyl)phenyl)-1-methyl- 1H-pyrazolo[3,4-d]pyrimidin-6-yl)- 2-methyl-2,8-diazaspiro[4.5]decan- 1-one | 497.6 |
| 18.09 | 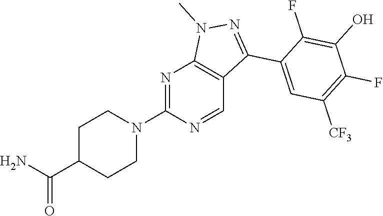 [SMILES: Cn1nc(-c2cc(C(F)(F)F)c(F)c(O)c2F)c2cnc(N3CCC(C(N)=O)CC3)nc21] | 1-(3-(2,4-Difluoro-3-hydroxy-5- (trifluoromethyl)phenyl)-1-methyl- 1H-pyrazolo[3,4-d]pyrimidin-6- yl)piperidine-4-carboxamide | 457.5 |
| 18.10 | 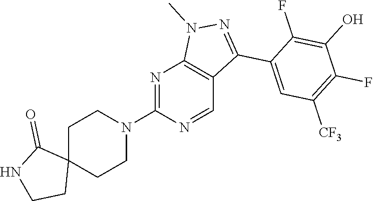 [SMILES: Cn1nc(-c2cc(C(F)(F)F)c(F)c(O)c2F)c2cnc(N3CCC4(CCNC4=O)CC3)nc21] | 8-(3-(2,4-Difluoro-3-hydroxy-5- (trifluoromethyl)phenyl)-1-methyl- 1H-pyrazolo[3,4-d]pyrimidin-6-yl)- 2, 8-diazaspiro[4.5]decan-1-one | 483.5 |
| 18.11 (1,3,4) | 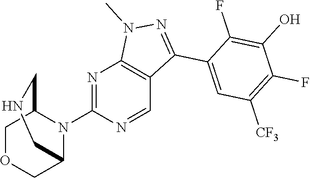 [SMILES: Cn1nc(-c2cc(C(F)(F)F)c(F)c(O)c2F)c2cnc(N3[C@@H]4CNC[C@H]3COC4)nc21] | 3-(6-((1R,5S)-3-oxa-7,9- diazabicyclo[3.3.1]nonan-9-y1)-1- methyl-1H-pyrazolo[3,4- d]pyrimidin-3-y1)-2,6-difluoro-5- (trifluoromethyl)phenol | 457.6 |
| 18.12 | 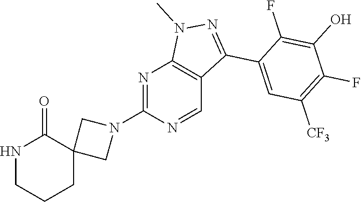 [SMILES: Cn1nc(-c2cc(C(F)(F)F)c(F)c(O)c2F)c2cnc(N3CC4(CCCNC4=O)C3)nc21] | 2-(3-(2,4-Difluoro-3-hydroxy-5- (trifluoromethyl)phenyl)-1-methyl- 1H-pyrazolo[3,4-d]pyrimidin-6-yl)- 2,6-diazaspiro[3.5]nonan-5-one | 469.5 |
| 18.13 (2) | 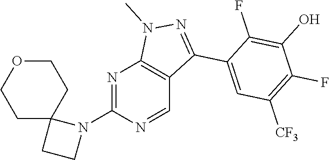 [SMILES: Cn1nc(-c2cc(C(F)(F)F)c(F)c(O)c2F)c2cnc(N3CCC34CCOCC4)nc21] | 2,6-Difluoro-3-(1-methy1-6-(7-oxa- 1-azaspiro[3.5]nonan-1-y1)-1H- pyrazolo[3,4-d]pyrimidin-3-y1)-5- (trifluoromethyl)phenol | 456.5 |
| 18.14 (2) | 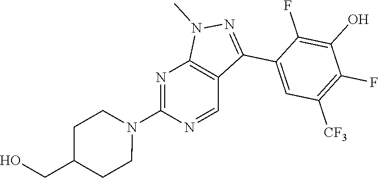 [SMILES: Cn1nc(-c2cc(C(F)(F)F)c(F)c(O)c2F)c2cnc(N3CCC(CO)CC3)nc21] | 2,6-Difluoro-3-(6-(4- (hydroxymethyl)piperidin-1-y1)-1- methyl-1H-pyrazolo[3,4- d]pyrimidin-3-y1)-5- (trifluoromethyl)phenol | 444.5 |
| 18.15 (2) | 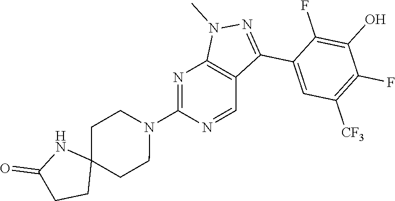 [SMILES: Cn1nc(-c2cc(C(F)(F)F)c(F)c(O)c2F)c2cnc(N3CCC4(CCC(=O)N4)CC3)nc21] | 8-(3-(2,4-Difluoro-3-hydroxy-5- (trifluoromethyl)phenyl)-1-methyl- 1H-pyrazolo[3,4-d]pyrimidin-6-yl)- 1,8-diazaspiro[4.5]decan-2-one | 483.3 |
| 18.16 (2) | 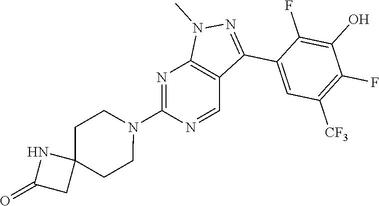 [SMILES: Cn1nc(-c2cc(C(F)(F)F)c(F)c(O)c2F)c2cnc(N3CCC4(CC3)CC(=O)N4)nc21] | 7-(3-(2,4-Difluoro-3-hydroxy-5- (trifluoromethyl)phenyl)-1-methyl- 1H-pyrazolo[3,4-d]pyrimidin-6-yl)- 1,7-diazaspiro[3.5]nonan-2-one | 469.4 |
| 18.17 (2) | 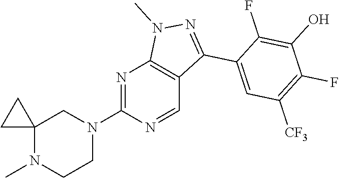 [SMILES: CN1CCN(c2ncc3c(-c4cc(C(F)(F)F)c(F)c(O)c4F)nn(C)c3n2)CC12CC2] | 2,6-Difluoro-3-(1-methyl-6-(4- methyl-4,7-diazaspiro[2.5]octan-7- y1)-1H-pyrazolo[3,4-d]pyrimidin-3- y1)-5-(trifluoromethyl)phenol | 455.6 |
| 18.18 (2) | 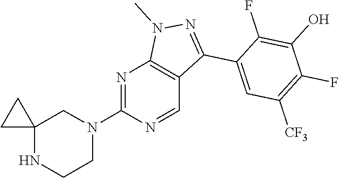 [SMILES: Cn1nc(-c2cc(C(F)(F)F)c(F)c(O)c2F)c2cnc(N3CCNC4(CC4)C3)nc21] | 2,6-Difluoro-3-(1-methyl-6-(4,7- diazaspiro[2.5]octan-7-y1)-1H- pyrazolo[3,4-d]pyrimidin-3-y1)-5- (trifluoromethyl)phenol | 441.6 |
| 18.19 (2) | 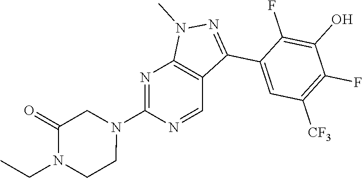 [SMILES: CCN1CCN(c2ncc3c(-c4cc(C(F)(F)F)c(F)c(O)c4F)nn(C)c3n2)CC1=O] | 4-(3-(2,4-Difluoro-3-hydroxy-5- (trifluoromethyl)phenyl)-1-methyl- 1H-pyrazolo[3,4-d]pyrimidin-6-yl)- 1-ethylpiperazin-2-one | 457.4 |
| 18.20 (2) | 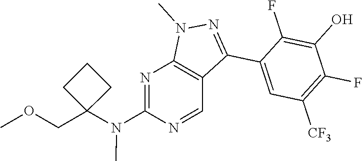 [SMILES: COCC1(N(C)c2ncc3c(-c4cc(C(F)(F)F)c(F)c(O)c4F)nn(C)c3n2)CCC1] | 2,6-Difluoro-3-(6-((1- (methoxymethyl)cyclobutyl)(methy 1)amino)-1-methy1-1H- pyrazolo[3,4-d]pyrimidin-3-y1)-5- (trifluoromethyl)phenol | 458.7 |
| 18.21 (2) | 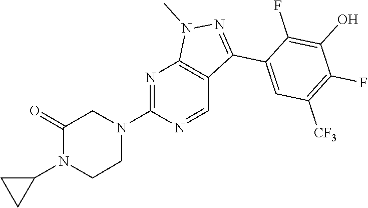 [SMILES: Cn1nc(-c2cc(C(F)(F)F)c(F)c(O)c2F)c2cnc(N3CCN(C4CC4)C(=O)C3)nc21] | 1-Cyclopropyl-4-(3-(2,4-difluoro-3- hydroxy-5- (trifluoromethyl)phenyl)-1-methyl- 1H-pyrazolo[3,4-d]pyrimidin-6- y1)piperazin-2-one | 469.4 |
| 18.22 (2) | 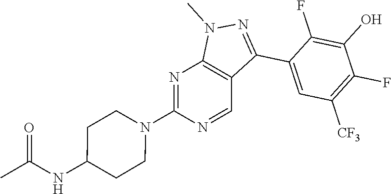 [SMILES: CC(=O)NC1CCN(c2ncc3c(-c4cc(C(F)(F)F)c(F)c(O)c4F)nn(C)c3n2)CC1] | N-(1-(3-(2,4-Difluoro-3-hydroxy-5- (trifluoromethyl)phenyl)-1-methyl- 1H-pyrazolo[3,4-d]pyrimidin-6- y1)piperidin-4-yl)acetamide | 471.5 |
| 18.23 (2) | 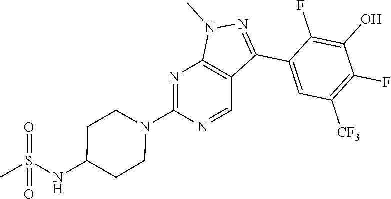 [SMILES: Cn1nc(-c2cc(C(F)(F)F)c(F)c(O)c2F)c2cnc(N3CCC(NS(C)(=O)=O)CC3)nc21] | N-(1-(3-(2,4-Difluoro-3-hydroxy-5- (trifluoromethyl)phenyl)-1-methyl- 1H-pyrazolo[3,4-d]pyrimidin-6- yl)piperidin-4- yl)methanesulfonamide | 507.6 |
| 18.24 (1,2,4) | 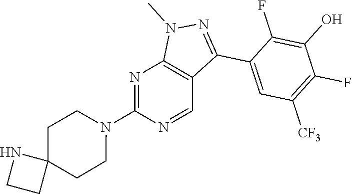 [SMILES: Cn1nc(-c2cc(C(F)(F)F)c(F)c(O)c2F)c2cnc(N3CCC4(CCN4)CC3)nc21] | 2,6-Difluoro-3-(1-methyl-6-(1,7- diazaspiro[3.5]nonan-7-yl)-1H- pyrazolo[3,4-d]pyrimidin-3-y1)-5- (trifluoromethyl)phenol | 455.6 |
| 18.25 (2) | 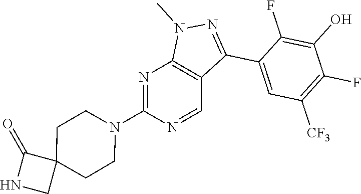 [SMILES: Cn1nc(-c2cc(C(F)(F)F)c(F)c(O)c2F)c2cnc(N3CCC4(CC3)CNC4=O)nc21] | 7-(3-(2,4-Difluoro-3-hydroxy-5- (trifluoromethyl)phenyl)-1-methyl- 1H-pyrazolo[3,4-d]pyrimidin-6-yl)- 2,7-diazaspiro[3.5]nonan-1-one | 469.6 |
| 18.26 (2) | 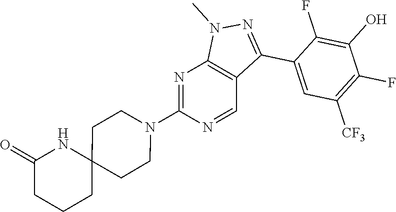 [SMILES: Cn1nc(-c2cc(C(F)(F)F)c(F)c(O)c2F)c2cnc(N3CCC4(CCCC(=O)N4)CC3)nc21] | 9-(3-(2,4-Difluoro-3-hydroxy-5- (trifluoromethyl)phenyl)-1-methyl- 1H-pyrazolo[3,4-d]pyrimidin-6-yl)- 1,9-diazaspiro[5.5]undecan-2-one | 497.4 |
| 18.27 (2) | 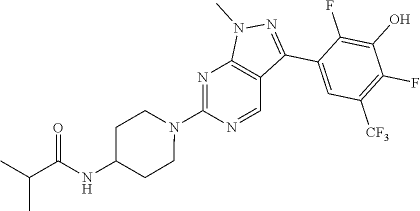 [SMILES: CC(C)C(=O)NC1CCN(c2ncc3c(-c4cc(C(F)(F)F)c(F)c(O)c4F)nn(C)c3n2)CC1] | N-(1-(3-(2,4-Difluoro-3-hydroxy-5- (trifluoromethyl)phenyl)-1-methyl- 1H-pyrazolo[3,4-d]pyrimidin-6- y1)piperidin-4-yl)isobutyramide | 499.7 |
| 18.28 (2) | 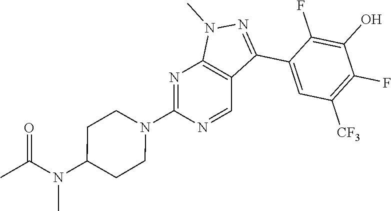 [SMILES: CC(=O)N(C)C1CCN(c2ncc3c(-c4cc(C(F)(F)F)c(F)c(O)c4F)nn(C)c3n2)CC1] | N-(1-(3-(2,4-Difluoro-3-hydroxy-5- (trifluoromethyl)phenyl)-1-methyl- 1H-pyrazolo[3,4-d]pyrimidin-6- yl)piperidin-4-y1)-N- methylacetamide | 485.6 |
| 18.29 (1,2,4) | 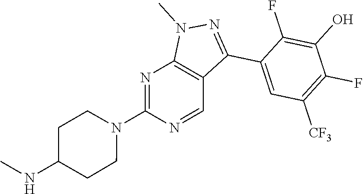 [SMILES: CNC1CCN(c2ncc3c(-c4cc(C(F)(F)F)c(F)c(O)c4F)nn(C)c3n2)CC1] | 2,6-Difluoro-3-(1-methyl-6-(4- (methylamino)piperidin-1-yl)-1H- pyrazolo[3,4-d]pyrimidin-3-y1)-5- (trifluoromethyl)phenol | 443.6 |
| 18.30 (2) | 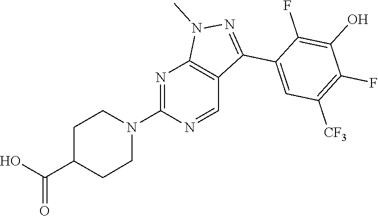 [SMILES: Cn1nc(-c2cc(C(F)(F)F)c(F)c(O)c2F)c2cnc(N3CCC(C(=O)O)CC3)nc21] | 1-(3-(2,4-Difluoro-3-hydroxy-5- (trifluoromethyl)phenyl)-1-methyl- 1H-pyrazolo[3,4-d]pyrimidin-6- y1)piperidine-4-carboxylic Acid | 458.6 |
| 18.31 (2) | 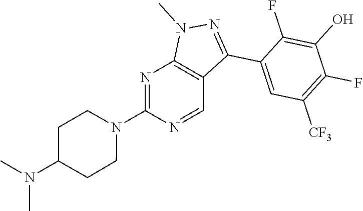 [SMILES: CN(C)C1CCN(c2ncc3c(-c4cc(C(F)(F)F)c(F)c(O)c4F)nn(C)c3n2)CC1] | 3-(6-(4-(Dimethylamino)piperidin- 1-y1)-1-methyl-1H-pyrazolo[3,4- d]pyrimidin-3-y1)-2,6-difluoro-5- (trifluoromethyl)phenol | 457.7 |
| 18.32 (2) | 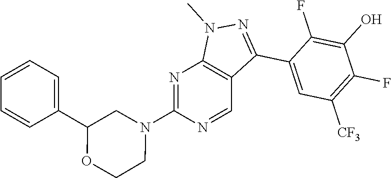 [SMILES: Cn1nc(-c2cc(C(F)(F)F)c(F)c(O)c2F)c2cnc(N3CCOC(c4ccccc4)C3)nc21] | 2,6-Difluoro-3-(1-methyl-6-(2- phenylmorpholino)-1H- pyrazolo[3,4-d]pyrimidin-3-y1)-5- (trifluoromethyl)phenol | 492.5 |
| 18.33 (2) | 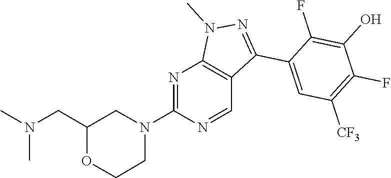 [SMILES: CN(C)CC1CN(c2ncc3c(-c4cc(C(F)(F)F)c(F)c(O)c4F)nn(C)c3n2)CCO1] | 3-(6-(2- ((Dimethylamino)methyl) morpholino)-1-methyl-1H- pyrazolo[3,4- d]pyrimidin-3-y1)-2,6-difluoro-5- (trifluoromethyl)phenol | 473.7 |
| 18.34 (2) | 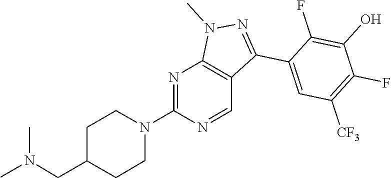 [SMILES: CN(C)CC1CCN(c2ncc3c(-c4cc(C(F)(F)F)c(F)c(O)c4F)nn(C)c3n2)CC1] | 3-(6-(4- ((Dimethylamino)methyl)piperidin- 1-y1)-1-methyl-1H-pyrazolo[3,4- d]pyrimidin-3-y1)-2,6-difluoro-5- (trifluoromethyl)phenol | 471.7 |
| 18.35 (2) | 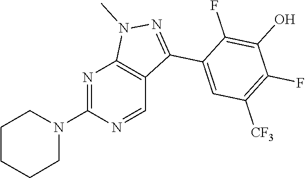 [SMILES: Cn1nc(-c2cc(C(F)(F)F)c(F)c(O)c2F)c2cnc(N3CCCCC3)nc21] | 2,6-Difluoro-3-(1-methyl-6- (piperidin-1-yl)-1H-pyrazolo[3,4- d]pyrimidin-3-y1)-5- (trifluoromethyl)phenol | 414.7 |
| 18.36 (2) | 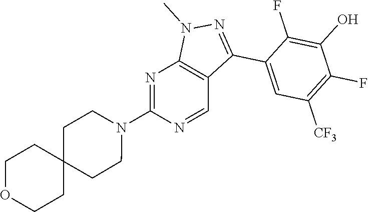 [SMILES: Cn1nc(-c2cc(C(F)(F)F)c(F)c(O)c2F)c2cnc(N3CCC4(CCOCC4)CC3)nc21] | 2,6-Difluoro-3-(1-methyl-6-(3-oxa- 9-azaspiro[5.5]undecan-9-yl)-1H- pyrazolo[3,4-d]pyrimidin-3-y1)-5- (trifluoromethyl)phenol | 484.8 |
| 18.37 (2) | 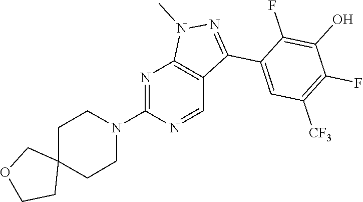 [SMILES: Cn1nc(-c2cc(C(F)(F)F)c(F)c(O)c2F)c2cnc(N3CCC4(CCOC4)CC3)nc21] | 2,6-Difluoro-3-(1-methyl-6-(2-oxa- 8-azaspiro[4.5]decan-8-yl)-1H- pyrazolo[3,4-d]pyrimidin-3-y1)-5- (trifluoromethyl)phenol | 470.5 |
| 18.38 (2,5) | 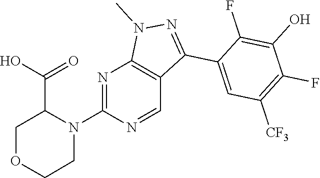 [SMILES: Cn1nc(-c2cc(C(F)(F)F)c(F)c(O)c2F)c2cnc(N3CCOCC3C(=O)O)nc21] | 4-(3-(2,4-Difluoro-3-hydroxy-5- (trifluoromethyl)phenyl)-1-methyl- 1H-pyrazolo[3,4-d]pyrimidin-6- yl)morpholine-3-carboxylic Acid | 460.3 |
| 18.39 (2) | 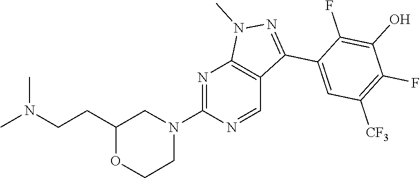 [SMILES: CN(C)CCC1CN(c2ncc3c(-c4cc(C(F)(F)F)c(F)c(O)c4F)nn(C)c3n2)CCO1] | 3-(6-(2-(2- (Dimethylamino)ethyl)morpholino)- 1-methyl-1H-pyrazolo[3,4- d]pyrimidin-3-y1)-2,6-difluoro-5- (trifluoromethyl)phenol | 487.7 |
| 18.40 (2) | 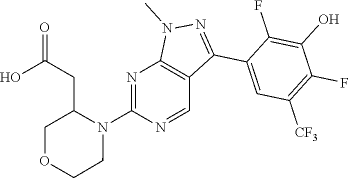 [SMILES: Cn1nc(-c2cc(C(F)(F)F)c(F)c(O)c2F)c2cnc(N3CCOCC3CC(=O)O)nc21] | 2-(4-(3-(2,4-Difluoro-3-hydroxy-5- (trifluoromethyl)phenyl)-1-methyl- 1H-pyrazolo[3,4-d]pyrimidin-6- yl)morpholin-3-yl)acetic Acid | 474.4 |
| 18.41 (2) | 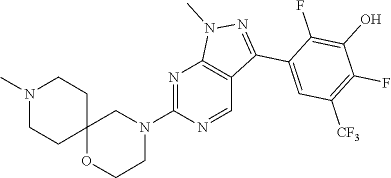 [SMILES: CN1CCC2(CC1)CN(c1ncc3c(-c4cc(C(F)(F)F)c(F)c(O)c4F)nn(C)c3n1)CCO2] | 2,6-Difluoro-3-(1-methyl-6-(9- methyl-1-oxa-4,9- diazaspiro[5.5]undecan-4-y1)-1H- pyrazolo[3,4-d]pyrimidin-3-y1)-5- (trifluoromethyl)phenol | 499.8 |
| 18.42 (2) | 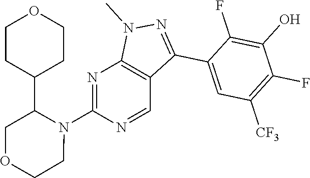 [SMILES: Cn1nc(-c2cc(C(F)(F)F)c(F)c(O)c2F)c2cnc(N3CCOCC3C3CCOCC3)nc21] | 2,6-Difluoro-3-(1-methyl-6-(3- (tetrahydro-2H-pyran-4- yl)morpholino)-1H-pyrazolo[3,4- d]pyrimidin-3-y1)-5- (trifluoromethyl)phenol | 500.6 |
| 18.43 (2,4) | 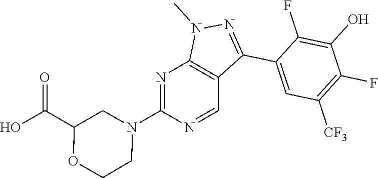 [SMILES: Cn1nc(-c2cc(C(F)(F)F)c(F)c(O)c2F)c2cnc(N3CCOC(C(=O)O)C3)nc21] | 4-(3-(2,4-Difluoro-3-hydroxy-5- (trifluoromethyl)phenyl)-1-methyl- 1H-pyrazolo[3,4-d]pyrimidin-6- yl)morpholine-2-carboxylic Acid | 460.5 |
| 18.44 (2) | 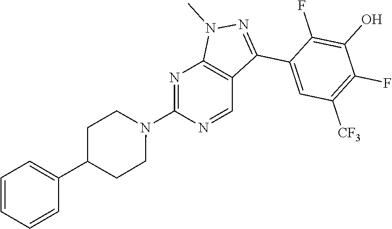 [SMILES: Cn1nc(-c2cc(C(F)(F)F)c(F)c(O)c2F)c2cnc(N3CCC(c4ccccc4)CC3)nc21] | 2,6-Difluoro-3-(1-methyl-6-(4- phenylpiperidin-1-y1)-1H- pyrazolo[3,4-d]pyrimidin-3-y1)-5- (trifluoromethyl)phenol | 490.6 |
| 18.45 (2) | 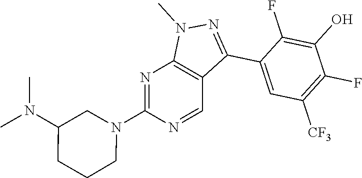 [SMILES: CN(C)C1CCCN(c2ncc3c(-c4cc(C(F)(F)F)c(F)c(O)c4F)nn(C)c3n2)C1] | 3-(6-(3-(Dimethylamino)piperidin- 1-y1)-1-methyl-1H-pyrazolo[3,4- d]pyrimidin-3-y1)-2,6-difluoro-5- (trifluoromethyl)phenol | 457.4 |
| 18.46 (2) | 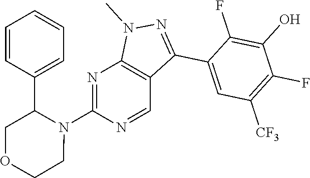 [SMILES: Cn1nc(-c2cc(C(F)(F)F)c(F)c(O)c2F)c2cnc(N3CCOCC3c3ccccc3)nc21] | 2,6-Difluoro-3-(1-methyl-6-(3- phenylmorpholino)-1H- pyrazolo[3,4-d]pyrimidin-3-y1)-5- (trifluoromethyl)phenol | 492.5 |
| 18.47 (2) | 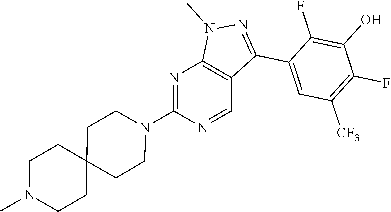 [SMILES: CN1CCC2(CC1)CCN(c1ncc3c(-c4cc(C(F)(F)F)c(F)c(O)c4F)nn(C)c3n1)CC2] | 2,6-Difluoro-3-(1-methyl-6-(9- methyl-3,9-diazaspiro[5.5]undecan- 3-y1)-1H-pyrazolo[3,4-d]pyrimidin- 3-y1)-5-(trifluoromethyl)phenol | 497.6 |
| 18.48 (2) | 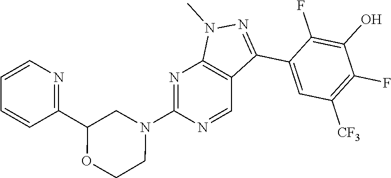 [SMILES: Cn1nc(-c2cc(C(F)(F)F)c(F)c(O)c2F)c2cnc(N3CCOC(c4ccccn4)C3)nc21] | 2,6-Difluoro-3-(1-methyl-6-(2- (pyridin-2-yl)morpholino)-1H- pyrazolo[3,4-d]pyrimidin-3-y1)-5- (trifluoromethyl)phenol | 493.5 |
| 18.49 (2) | 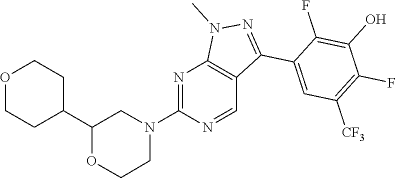 [SMILES: Cn1nc(-c2cc(C(F)(F)F)c(F)c(O)c2F)c2cnc(N3CCOC(C4CCOCC4)C3)nc21] | 2,6-Difluoro-3-(1-methyl-6-(2- (tetrahydro-2H-pyran-4- yl)morpholino)-1H-pyrazolo[3,4- d]pyrimidin-3-y1)-5- (trifluoromethyl)phenol | 500.5 |
| 18.50 (2) | 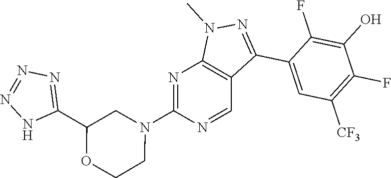 [SMILES: Cn1nc(-c2cc(C(F)(F)F)c(F)c(O)c2F)c2cnc(N3CCOC(c4nnn[nH]4)C3)nc21] | 3-(6-(2-(1H-Tetrazol-5- yl)morpholino)-1-methyl-1H- pyrazolo[3,4-d]pyrimidin-3-y1)-2,6- difluoro-5-(trifluoromethyl)phenol | 484.4 |
| 18.51 (2) | 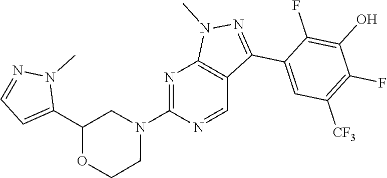 [SMILES: Cn1nccc1C1CN(c2ncc3c(-c4cc(C(F)(F)F)c(F)c(O)c4F)nn(C)c3n2)CCO1] | 2,6-Difluoro-3-(1-methyl-6-(2-(1- methyl-1H-pyrazol-5- yl)morpholino)-1H-pyrazolo[3,4- d]pyrimidin-3-y1)-5- (trifluoromethyl)phenol | 496.4 |
| 18.52 (2) | 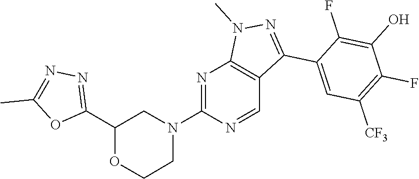 [SMILES: Cc1nnc(C2CN(c3ncc4c(-c5cc(C(F)(F)F)c(F)c(O)c5F)nn(C)c4n3)CCO2)o1] | 2,6-Difluoro-3-(1-methyl-6-(2-(5- methyl-1,3,4-oxadiazol-2- y1)morpholino)-1H-pyrazolo[3,4- d]pyrimidin-3-y1)-5- (trifluoromethyl)phenol | 498.4 |
| 18.53 (2) | 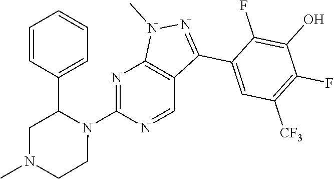 [SMILES: CN1CCN(c2ncc3c(-c4cc(C(F)(F)F)c(F)c(O)c4F)nn(C)c3n2)C(c2ccccc2)C1] | 2,6-Difluoro-3-(1-methyl-6-(4- methyl-2-phenylpiperazin-1-yl)- 1H-pyrazolo[3,4-d]pyrimidin-3-yl)- 5-(trifluoromethyl)phenol | 505.8 |
| 18.54 (2) | 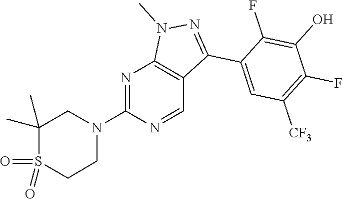 [SMILES: Cn1nc(-c2cc(C(F)(F)F)c(F)c(O)c2F)c2cnc(N3CCS(=O)(=O)C(C)(C)C3)nc21] | 4-(3-(2,4-Difluoro-3-hydroxy-5- (trifluoromethyl)phenyl)-1-methyl- 1H-pyrazolo[3,4-d]pyrimidin-6-yl)- 2,2-dimethylthiomorpholine 1,1- dioxide | 492.4 |
| 18.55 (2) | 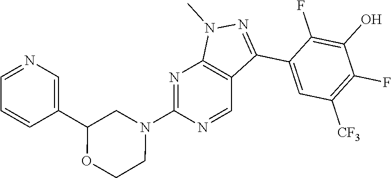 [SMILES: Cn1nc(-c2cc(C(F)(F)F)c(F)c(O)c2F)c2cnc(N3CCOC(c4cccnc4)C3)nc21] | 2,6-Difluoro-3-(1-methyl-6-(2- (pyridin-3-y1)morpholino)-1H- pyrazolo[3,4-d]pyrimidin-3-y1)-5- (trifluoromethyl)phenol | 493.4 |
| 18.56 (2) | 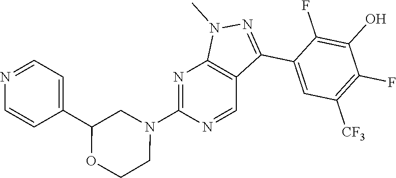 [SMILES: Cn1nc(-c2cc(C(F)(F)F)c(F)c(O)c2F)c2cnc(N3CCOC(c4ccncc4)C3)nc21] | 2,6-Difluoro-3-(1-methyl-6-(2- (pyridin-4-y1)morpholino)-1H- pyrazolo[3,4-d]pyrimidin-3-y1)-5- (trifluoromethyl)phenol | 493.4 |
| 18.57 (2) | 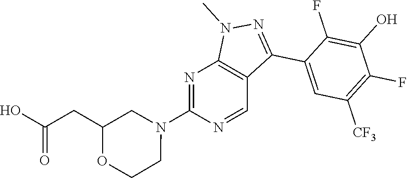 [SMILES: Cn1nc(-c2cc(C(F)(F)F)c(F)c(O)c2F)c2cnc(N3CCOC(CC(=O)O)C3)nc21] | 2-(4-(3-(2,4-Difluoro-3-hydroxy-5- (trifluoromethyl)phenyl)-1-methyl- 1H-pyrazolo[3,4-d]pyrimidin-6- yl)morpholin-2-yl)acetic Acid | 474.3 |
| 18.58 (2) | 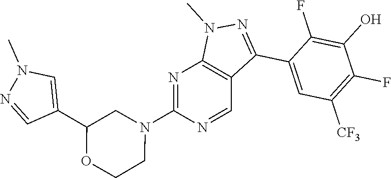 [SMILES: Cn1cc(C2CN(c3ncc4c(-c5cc(C(F)(F)F)c(F)c(O)c5F)nn(C)c4n3)CCO2)cn1] | 2,6-Difluoro-3-(1-methyl-6-(2-(1- methyl-1H-pyrazol-4- yl)morpholino)-1H-pyrazolo[3,4- d]pyrimidin-3-y1)-5- (trifluoromethyl)phenol | 496.4 |
| 18.59 (2) | 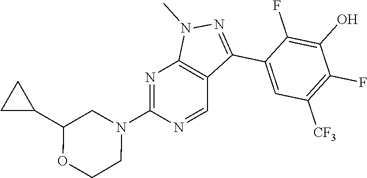 [SMILES: Cn1nc(-c2cc(C(F)(F)F)c(F)c(O)c2F)c2cnc(N3CCOC(C4CC4)C3)nc21] | 3-(6-(2-Cyclopropylmorpholino)-1- methyl-1H-pyrazolo[3,4- d]pyrimidin-3-y1)-2,6-difluoro-5- (trifluoromethyl)phenol | 456.5 |
| 18.60 (2) | 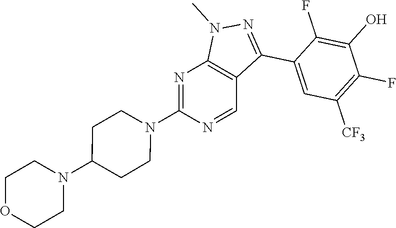 [SMILES: Cn1nc(-c2cc(C(F)(F)F)c(F)c(O)c2F)c2cnc(N3CCC(N4CCOCC4)CC3)nc21] | 2,6-Difluoro-3-(1-methyl-6-(4- morpholinopiperidin-1-yl)-1H- pyrazolo[3,4-d]pyrimidin-3-y1)-5- (trifluoromethyl)phenol | 499.4 |
| 18.61 (2) | 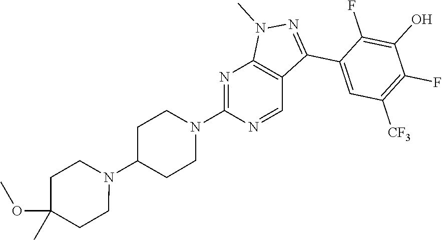 [SMILES: COC1(C)CCN(C2CCN(c3ncc4c(-c5cc(C(F)(F)F)c(F)c(O)c5F)nn(C)c4n3)CC2)CC1] | 2,6-Difluoro-3-(6-(4-methoxy-4- methylpiperidin-1-yl)-1-methyl- 1H-pyrazolo[3,4-d]pyrimidin-3-yl)- 5-(trifluoromethyl)phenol | 458.7 |
| 18.62 (2) | 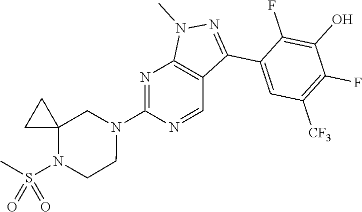 [SMILES: Cn1nc(-c2cc(C(F)(F)F)c(F)c(O)c2F)c2cnc(N3CCN(S(C)(=O)=O)C4(CC4)C3)nc21] | 2,6-Difluoro-3-(1-methyl-6-(4- (methylsulfony1)-4,7- diazaspiro [2.5]octan-7-y1)-1H- pyrazolo[3,4-d]pyrimidin-3-y1)-5- (trifluoromethyl)phenol | 519.4 |
| 18.63 (2) | 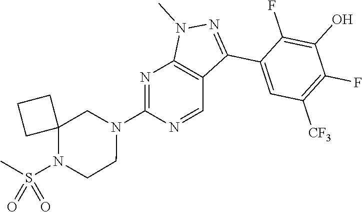 [SMILES: Cn1nc(-c2cc(C(F)(F)F)c(F)c(O)c2F)c2cnc(N3CCN(S(C)(=O)=O)C4(CCC4)C3)nc21] | 2,6-Difluoro-3-(1-methyl-6-(5- (methylsulfony1)-5,8- diazaspiro[3.5]nonan-8-yl)-1H- pyrazolo[3,4-d]pyrimidin-3-y1)-5- (trifluoromethyl)phenol | 533.6 |
| 18.64 (2) | 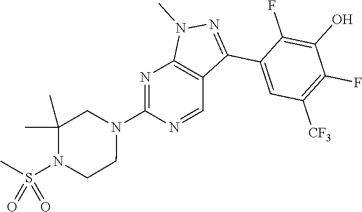 [SMILES: Cn1nc(-c2cc(C(F)(F)F)c(F)c(O)c2F)c2cnc(N3CCN(S(C)(=O)=O)C(C)(C)C3)nc21] | 3-(6-(3,3-Dimethyl-4- (methylsulfonyl)piperazin-1-y1)-1- methyl-1H-pyrazolo[3,4- d]pyrimidin-3-y1)-2,6-difluoro-5- (trifluoromethyl)phenol | 521.4 |
| 18.65 (2) | 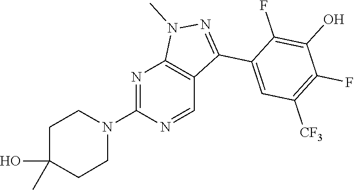 [SMILES: Cn1nc(-c2cc(C(F)(F)F)c(F)c(O)c2F)c2cnc(N3CCC(C)(O)CC3)nc21] | 1-(3-(2,4-Difluoro-3-hydroxy-5- (trifluoromethyl)phenyl)-1-methyl- 1H-pyrazolo[3,4-d]pyrimidin-6-yl)- 4-methylpiperidin-4-ol | 444.3 |
| 18.66 (2) | 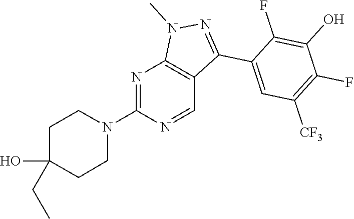 [SMILES: CCC1(O)CCN(c2ncc3c(-c4cc(C(F)(F)F)c(F)c(O)c4F)nn(C)c3n2)CC1] | 1-(3-(2,4-Difluoro-3-hydroxy-5- (trifluoromethyl)phenyl)-1-methyl- 1H-pyrazolo[3,4-d]pyrimidin-6-yl)- 4-ethylpiperidin-4-ol | 458.5 |
| 18.67 (2) | 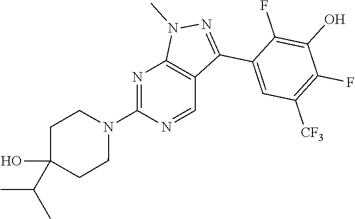 [SMILES: CC(C)C1(O)CCN(c2ncc3c(-c4cc(C(F)(F)F)c(F)c(O)c4F)nn(C)c3n2)CC1] | 1-(3-(2,4-Difluoro-3-hydroxy-5- (trifluoromethyl)phenyl)-1-methyl- 1H-pyrazolo[3,4-d]pyrimidin-6-yl)- 4-isopropylpiperidin-4-ol | 472.3 |
| 18.68 (2) | 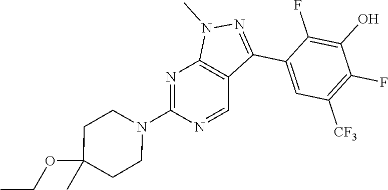 [SMILES: CCOC1(C)CCN(c2ncc3c(-c4cc(C(F)(F)F)c(F)c(O)c4F)nn(C)c3n2)CC1] | 3-(6-(4-Ethoxy-4-methylpiperidin- 1-y1)-1-methyl-1H-pyrazolo[3,4- d]pyrimidin-3-y1)-2,6-difluoro-5- (trifluoromethyl)phenol | 472.6 |
| 18.69 (2) | 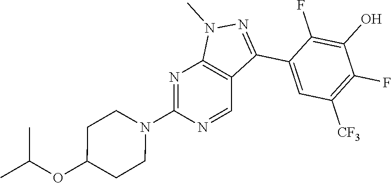 [SMILES: CC(C)OC1CCN(c2ncc3c(-c4cc(C(F)(F)F)c(F)c(O)c4F)nn(C)c3n2)CC1] | 2,6-Difluoro-3 -(6-(4- isopropoxypiperidin-1-yl)-1- methyl-1H-pyrazolo[3,4- d]pyrimidin-3-y1)-5- (trifluoromethyl)phenol | 472.6 |
| 18.70 (2) | 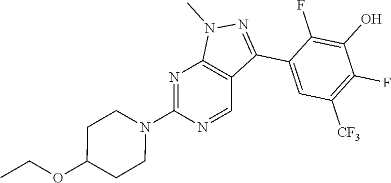 [SMILES: CCOC1CCN(c2ncc3c(-c4cc(C(F)(F)F)c(F)c(O)c4F)nn(C)c3n2)CC1] | 3-(6-(4-Ethoxypiperidin-1-y1)-1- methyl-1H-pyrazolo[3,4- d]pyrimidin-3-y1)-2,6-difluoro-5- (trifluoromethyl)phenol | 458.7 |
| 18.71 (2) | 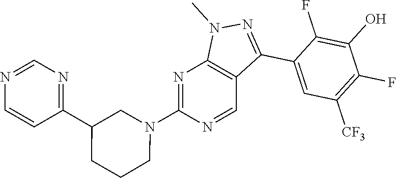 [SMILES: Cn1nc(-c2cc(C(F)(F)F)c(F)c(O)c2F)c2cnc(N3CCCC(c4ccncn4)C3)nc21] | 2,6-Difluoro-3-(1-methyl-6-(3- (pyrimidin-4-y1)piperidin-1-yl)-1H- pyrazolo[3,4-d]pyrimidin-3-y1)-5- (trifluoromethyl)phenol | 492.5 |
| 18.72 (2) | 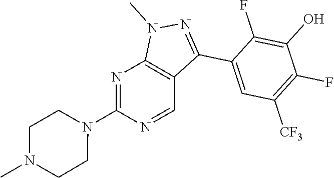 [SMILES: CN1CCN(c2ncc3c(-c4cc(C(F)(F)F)c(F)c(O)c4F)nn(C)c3n2)CC1] | 2,6-Difluoro-3-(1-methyl-6-(4- methylpiperazin-1-yl)-1H- pyrazolo[3,4-d]pyrimidin-3-y1)-5- (trifluoromethyl)phenol | 429.6 |
| 18.73 (2) | 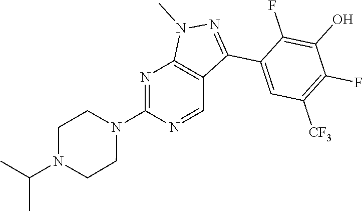 [SMILES: CC(C)N1CCN(c2ncc3c(-c4cc(C(F)(F)F)c(F)c(O)c4F)nn(C)c3n2)CC1] | 2,6-Difluoro-3 -(6-(4- isopropylpiperazin-1-yl)-1-methyl- 1H-pyrazolo[3,4-d]pyrimidin-3-yl)- 5-(trifluoromethyl)phenol | 457.7 |
| 18.74 (2) | 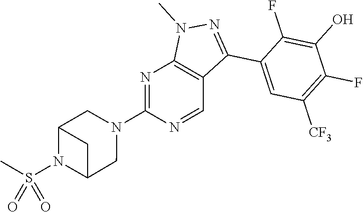 [SMILES: Cn1nc(-c2cc(C(F)(F)F)c(F)c(O)c2F)c2cnc(N3CC4CC(C3)N4S(C)(=O)=O)nc21] | 2,6-Difluoro-3-(1-methyl-6-(6- (methylsulfony1)-3,6- diazabicyclo[3.1.1]heptan-3-yl)- 1H-pyrazolo[3,4-d]pyrimidin-3-y1)- 5-(trifluoromethyl)phenol | 505.4 |
| 18.75 (2) | 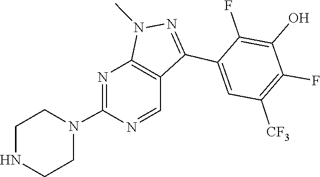 [SMILES: Cn1nc(-c2cc(C(F)(F)F)c(F)c(O)c2F)c2cnc(N3CCNCC3)nc21] | 2,6-Difluoro-3-(1-methyl-6- (piperazin-1-y1)-1H-pyrazolo[3,4- d]pyrimidin-3-y1)-5- (trifluoromethyl)phenol | 415.4 |
| 18.76 (2) | 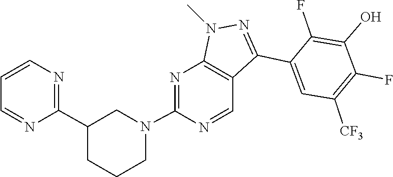 [SMILES: Cn1nc(-c2cc(C(F)(F)F)c(F)c(O)c2F)c2cnc(N3CCCC(c4ncccn4)C3)nc21] | 2,6-Difluoro-3-(1-methyl-6-(3- (pyrimidin-2-y1)piperidin-1-yl)-1H- pyrazolo[3,4-d]pyrimidin-3-y1)-5- (trifluoromethyl)phenol | 492.4 |
| 18.77 (2) | 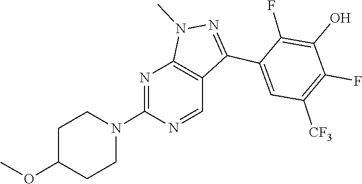 [SMILES: COC1CCN(c2ncc3c(-c4cc(C(F)(F)F)c(F)c(O)c4F)nn(C)c3n2)CC1] | 2,6-Difluoro-3-(6-(4- methoxypiperidin-1-y1)-1-methyl- 1H-pyrazolo[3,4-d]pyrimidin-3-yl)- 5-(trifluoromethyl)phenol | 444.4 |
| 18.78 (2,4) | 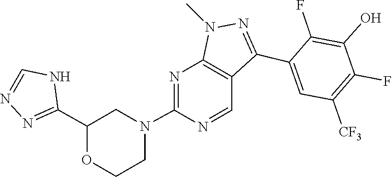 [SMILES: Cn1nc(-c2cc(C(F)(F)F)c(F)c(O)c2F)c2cnc(N3CCOC(c4nnc[nH]4)C3)nc21] | 3-(6-(2-(4H-1,2,4-Triazol-3- yl)morpholino)-1-methyl-1H- pyrazolo[3,4-d]pyrimidin-3-y1)-2,6- difluoro-5-(trifluoromethyl)phenol | 483.3 |
| 18.79 (2) | 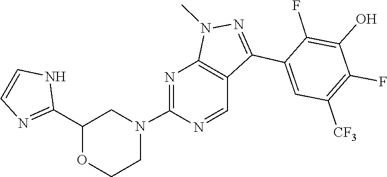 [SMILES: Cn1nc(-c2cc(C(F)(F)F)c(F)c(O)c2F)c2cnc(N3CCOC(c4ncc[nH]4)C3)nc21] | 3-(6-(2-(1H-Imidazol-2- yl)morpholino)-1-methyl-1H- pyrazolo[3,4-d]pyrimidin-3-y1)-2,6- difluoro-5-(trifluoromethyl)phenol | 482.3 |
| 18.80 (2) | 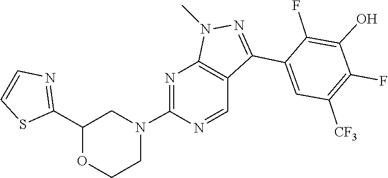 [SMILES: Cn1nc(-c2cc(C(F)(F)F)c(F)c(O)c2F)c2cnc(N3CCOC(c4nccs4)C3)nc21] | 2,6-Difluoro-3-(1-methyl-6-(2- (thiazol-2-yl)morpholino)-1H- pyrazolo[3,4-d]pyrimidin-3-y1)-5- (trifluoromethyl)phenol | 499.3 |
| 18.81 (2) | 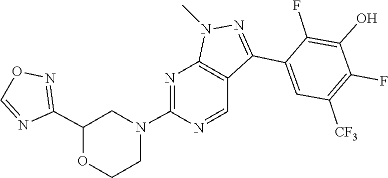 [SMILES: Cn1nc(-c2cc(C(F)(F)F)c(F)c(O)c2F)c2cnc(N3CCOC(c4ncon4)C3)nc21] | 3-(6-(2-(1,2,4-Oxadiazol-3- yl)morpholino)-1-methyl-1H- pyrazolo[3,4-d]pyrimidin-3-y1)-2,6- difluoro-5-(trifluoromethyl)phenol | 484.4 |
| 18.82 (2) | 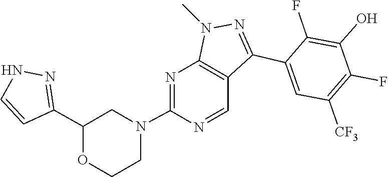 [SMILES: Cn1nc(-c2cc(C(F)(F)F)c(F)c(O)c2F)c2cnc(N3CCOC(c4cc[nH]n4)C3)nc21] | 3-(6-(2-(1H-Pyrazol-3- yl)morpholino)-1-methyl-1H- pyrazolo[3,4-d]pyrimidin-3-y1)-2,6- difluoro-5-(trifluoromethyl)phenol | 482.5 |
| 18.83 (2) | 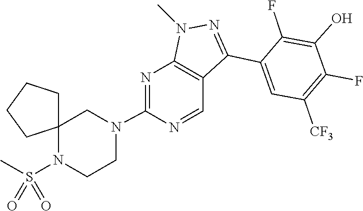 [SMILES: Cn1nc(-c2cc(C(F)(F)F)c(F)c(O)c2F)c2cnc(N3CCN(S(C)(=O)=O)C4(CCCC4)C3)nc21] | 2,6-Difluoro-3-(1-methyl-6-(6- (methylsulfonyl)-6,9- diazaspiro[4.5]decan-9-yl)-1H- pyrazolo[3,4-d]pyrimidin-3-y1)-5- (trifluoromethyl)phenol | 547.4 |
| 18.84 (2) | 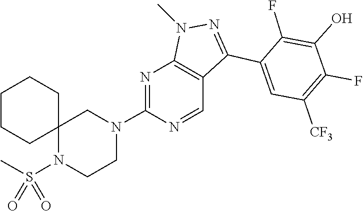 [SMILES: Cn1nc(-c2cc(C(F)(F)F)c(F)c(O)c2F)c2cnc(N3CCN(S(C)(=O)=O)C4(CCCCC4)C3)nc21] | 2,6-Difluoro-3-(1-methyl-6-(1- (methylsulfony1)-1,4- diazaspiro[5.5]undecan-4-yl)-1H- pyrazolo[3,4-d]pyrimidin-3-y1)-5- (trifluoromethyl)phenol | 561.4 |
| 18.85 (2) | 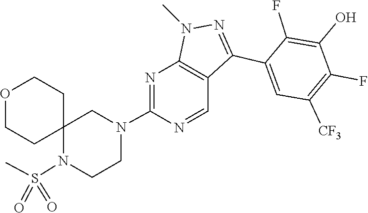 [SMILES: Cn1nc(-c2cc(C(F)(F)F)c(F)c(O)c2F)c2cnc(N3CCN(S(C)(=O)=O)C4(CCOCC4)C3)nc21] | 2,6-Difluoro-3-(1-methyl-6-(1- (methylsulfony1)-9-oxa-1,4- diazaspiro[5.5]undecan-4-y1)-1H- pyrazolo[3,4-d]pyrimidin-3-y1)-5- (trifluoromethyl)phenol | 563.4 |
| 18.86 (2) | 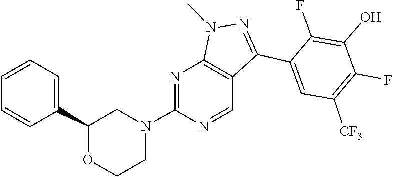 [SMILES: Cn1nc(-c2cc(C(F)(F)F)c(F)c(O)c2F)c2cnc(N3CCO[C@@H](c4ccccc4)C3)nc21] | (S)-2,6-Difluoro-3-(1-methyl-6-(2- phenylmorpholino)-1H- pyrazolo[3,4-d]pyrimidin-3-y1)-5- (trifluoromethyl)phenol | 492.4 |
| 18.87 (2) | 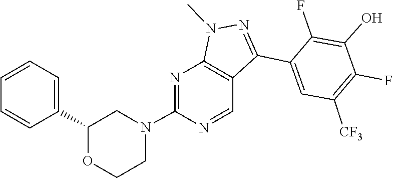 [SMILES: Cn1nc(-c2cc(C(F)(F)F)c(F)c(O)c2F)c2cnc(N3CCO[C@H](c4ccccc4)C3)nc21] | (R)-2,6-Difluoro-3-(1-methyl-6-(2- phenylmorpholino)-1H- pyrazolo[3,4-d]pyrimidin-3-y1)-5- (trifluoromethyl)phenol | 492.4 |
| 18.88 (2) | 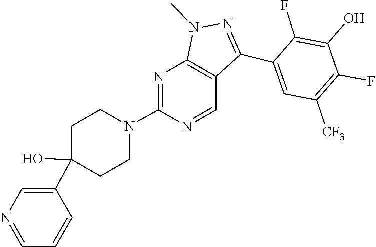 [SMILES: Cn1nc(-c2cc(C(F)(F)F)c(F)c(O)c2F)c2cnc(N3CCC(O)(c4cccnc4)CC3)nc21] | 1-(3-(2,4-Difluoro-3-hydroxy-5- (trifluoromethyl)phenyl)-1-methyl- 1H-pyrazolo[3,4-d]pyrimidin-6-yl)- 4-(pyridin-3-yl)piperidin-4-ol | 507.4 |
| 18.89 (2) | 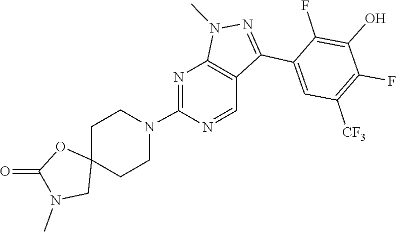 [SMILES: CN1CC2(CCN(c3ncc4c(-c5cc(C(F)(F)F)c(F)c(O)c5F)nn(C)c4n3)CC2)OC1=O] | 8-(3-(2,4-Difluoro-3-hydroxy-5- (trifluoromethyl)phenyl)-1-methyl- 1H-pyrazolo[3,4-d]pyrimidin-6-yl)- 3-methyl-1-oxa-3,8- diazaspiro[4.5]decan-2-one | 499.4 |
| 18.90 (2) | 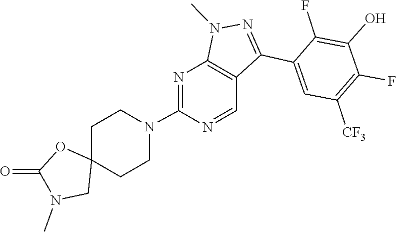 [SMILES: CN1CC2(CCN(c3ncc4c(-c5cc(C(F)(F)F)c(F)c(O)c5F)nn(C)c4n3)CC2)OC1=O] | 2,6-Difluoro-3-(1-methyl-6-(3- (pyrimidin-5-y1)piperidin-1-yl)-1H- pyrazolo[3,4-d]pyrimidin-3-y1)-5- (trifluoromethyl)phenol | 492.4 |
| 18.91 (2) | 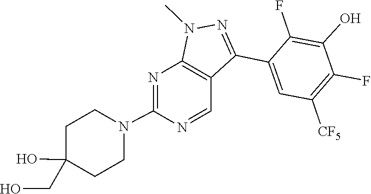 | 1-(3-(2,4-Difluoro-3-hydroxy-5- (trifluoromethyl)phenyl)-1-methyl- 1H-pyrazolo[3,4-d]pyrimidin-6-yl)- 4-(hydroxymethyl)piperidin-4-ol | 460.3 |
| 18.92 (2) | 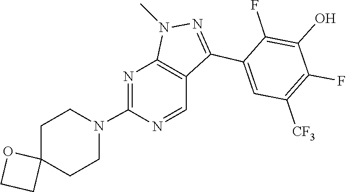 [SMILES: Cn1nc(-c2cc(C(F)(F)F)c(F)c(O)c2F)c2cnc(N3CCC4(CCO4)CC3)nc21] | 2,6-Difluoro-3-(1-methyl-6-(1-oxa- 7-azaspiro[3.5]nonan-7-y1)-1H- pyrazolo[3,4-d]pyrimidin-3-y1)-5- (trifluoromethyl)phenol | 456.4 |
| 18.93 (2) | 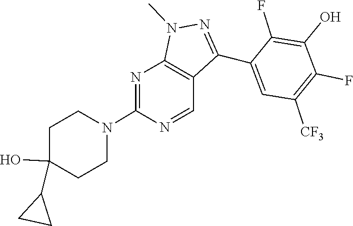 [SMILES: Cn1nc(-c2cc(C(F)(F)F)c(F)c(O)c2F)c2cnc(N3CCC(O)(C4CC4)CC3)nc21] | 4-Cyclopropyl-1-(3-(2,4-difluoro-3- hydroxy-5- (trifluoromethyl)phenyl)-1-methyl- 1H-pyrazolo[3,4-d]pyrimidin-6- y1)piperidin-4-ol | 470.4 |
| 18.94 (2) | 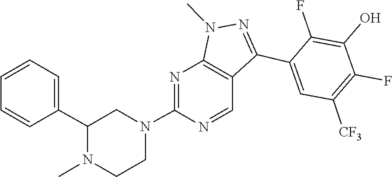 [SMILES: CN1CCN(c2ncc3c(-c4cc(C(F)(F)F)c(F)c(O)c4F)nn(C)c3n2)CC1c1ccccc1] | 2,6-Difluoro-3-(1-methyl-6-(4- methyl-3-phenylpiperazin-1-y1)- 1H-pyrazolo[3,4-d]pyrimidin-3-yl)- 5-(trifluoromethyl)phenol | 505.4 |
| 18.95 (2) | 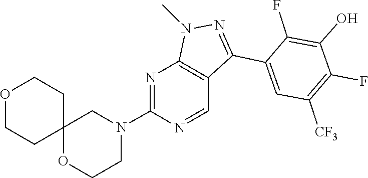 [SMILES: Cn1nc(-c2cc(C(F)(F)F)c(F)c(O)c2F)c2cnc(N3CCOC4(CCOCC4)C3)nc21] | 2,6-Difluoro-3-(1-methyl-6-(1,9- dioxa-4-azaspiro[5.5 ]undecan-4- y1)-1H-pyrazolo[3,4-d]pyrimidin-3- y1)-5-(trifluoromethyl)phenol | 486.4 |
| 18.96 (2) | 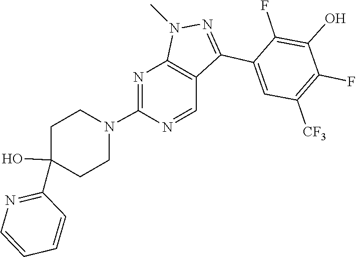 [SMILES: Cn1nc(-c2cc(C(F)(F)F)c(F)c(O)c2F)c2cnc(N3CCC(O)(c4ccccn4)CC3)nc21] | 1-(3-(2,4-Difluoro-3-hydroxy-5- (trifluoromethyl)phenyl)-1-methyl- 1H-pyrazolo[3,4-d]pyrimidin-6-yl)- 4-(pyridin-2-y1)piperidin-4-ol | 507.4 |
| 18.97 (2) | 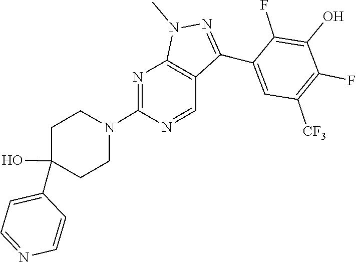 [SMILES: Cn1nc(-c2cc(C(F)(F)F)c(F)c(O)c2F)c2cnc(N3CCC(O)(c4ccncc4)CC3)nc21] | 1-(3-(2,4-Difluoro-3-hydroxy-5- (trifluoromethyl)phenyl)-1-methyl- 1H-pyrazolo[3,4-d]pyrimidin-6-yl)- 4-(pyridin-4-yl)piperidin-4-ol | 507.4 |
| 18.98 (2) | 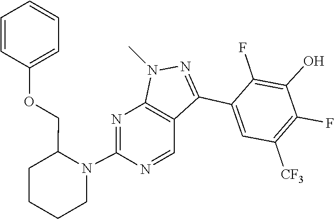 [SMILES: Cn1nc(-c2cc(C(F)(F)F)c(F)c(O)c2F)c2cnc(N3CCCCC3COc3ccccc3)nc21] | 2,6-Difluoro-3-(1-methyl-6-(2- (phenoxymethyl)piperidin-1-yl)- 1H-pyrazolo[3,4-d]pyrimidin-3-yl)- 5-(trifluoromethyl)phenol | 520.4 |
| 18.99 (2) | 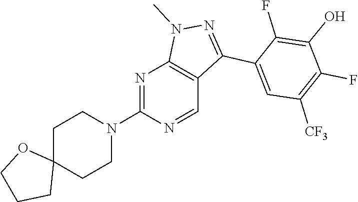 [SMILES: Cn1nc(-c2cc(C(F)(F)F)c(F)c(O)c2F)c2cnc(N3CCC4(CCCO4)CC3)nc21] | 2,6-Difluoro-3-(1-methyl-6-(1-oxa- 8-azaspiro[4.5]decan-8-yl)-1H- pyrazolo[3,4-d]pyrimidin-3-y1)-5- (trifluoromethyl)phenol | 470.4 |
| 18.100 (2) | 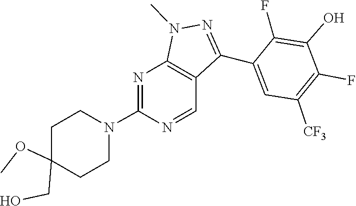 [SMILES: COC1(CO)CCN(c2ncc3c(-c4cc(C(F)(F)F)c(F)c(O)c4F)nn(C)c3n2)CC1] | 2,6-Difluoro-3-(6-(4- (hydroxymethyl)-4- methoxypiperidin-1-y1)-1-methyl- 1H-pyrazolo[3,4-d]pyrimidin-3-yl)- 5-(trifluoromethyl)phenol | 474.4 |
| 18.101 (2) | 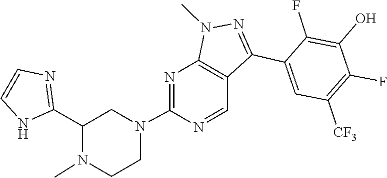 [SMILES: CN1CCN(c2ncc3c(-c4cc(C(F)(F)F)c(F)c(O)c4F)nn(C)c3n2)CC1c1ncc[nH]1] | 3-(6-(3-(1H-Imidazol-2-yl)-4- methylpiperazin-1-y1)-1-methyl- 1H-pyrazolo[3,4-d]pyrimidin-3-yl)- 2,6-difluoro-5- (trifluoromethyl)phenol | 495.4 |
| 18.102 (2,6) | 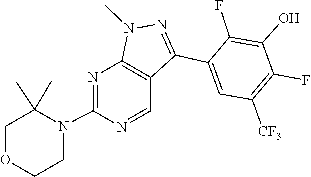 [SMILES: Cn1nc(-c2cc(C(F)(F)F)c(F)c(O)c2F)c2cnc(N3CCOCC3(C)C)nc21] | 3-(6-(3,3-Dimethylmorpholino)-1- methyl-1H-pyrazolo[3,4- d]pyrimidin-3-y1)-2,6-difluoro-5- (trifluoromethyl)phenol | 444.5 |
| 18.103 (2) | 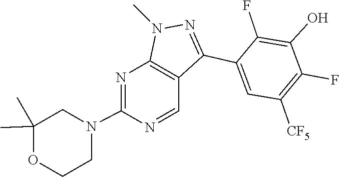 | 3-(6-(2,2-Dimethylmorpholino)-1- methyl-1H-pyrazolo[3,4- d]pyrimidin-3-y1)-2,6-difluoro-5- (trifluoromethyl)phenol | 444.5 |
| 18.104 (2) | 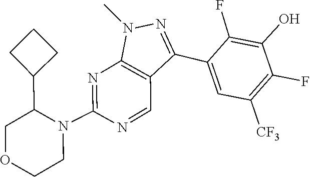 [SMILES: Cn1nc(-c2cc(C(F)(F)F)c(F)c(O)c2F)c2cnc(N3CCOCC3C3CCC3)nc21] | 3-(6-(3-Cyclobutylmorpholino)-1- methyl-1H-pyrazolo[3,4- d]pyrimidin-3-y1)-2,6-difluoro-5- (trifluoromethyl)phenol | 470.4 |
| 18.105 (2) | 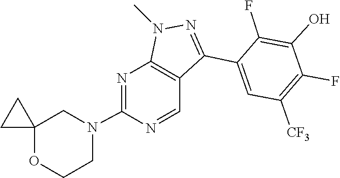 [SMILES: Cn1nc(-c2cc(C(F)(F)F)c(F)c(O)c2F)c2cnc(N3CCOC4(CC4)C3)nc21] | 2,6-Difluoro-3-(1-methyl-6-(4-oxa- 7-azaspiro[2.5]octan-7-y1)-1H- pyrazolo[3,4-d]pyrimidin-3-y1)-5- (trifluoromethyl)phenol | 442.5 |
| 18.106 (2,6) | 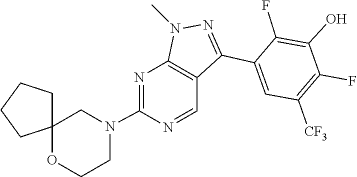 [SMILES: Cn1nc(-c2cc(C(F)(F)F)c(F)c(O)c2F)c2cnc(N3CCOC4(CCCC4)C3)nc21] | 2,6-Difluoro-3-(1-methyl-6-(6-oxa- 9-azaspiro[4.5]decan-9-yl)-1H- pyrazolo[3,4-d]pyrimidin-3-y1)-5- (trifluoromethyl)phenol | 470.4 |
| 18.107 (2,6) | 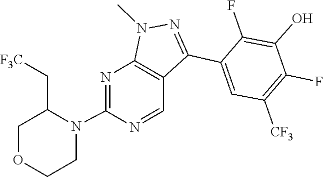 [SMILES: Cn1nc(-c2cc(C(F)(F)F)c(F)c(O)c2F)c2cnc(N3CCOCC3CC(F)(F)F)nc21] | 2,6-Difluoro-3-(1-methyl-6-(3- (2,2,2-trifluoroethyl)morpholino)- 1H-pyrazolo[3,4-d]pyrimidin-3-y1)- 5-(trifluoromethyl)phenol | 498.3 |
| 18.108 (2,6) | 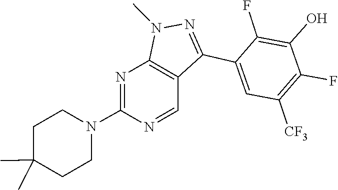 [SMILES: Cn1nc(-c2cc(C(F)(F)F)c(F)c(O)c2F)c2cnc(N3CCC(C)(C)CC3)nc21] | 3-(6-(4,4-Dimethylpiperidin-1-yl)- 1-methyl-1H-pyrazolo[3,4- d]pyrimidin-3-yl)-2,6-difluoro-5- (trifluoromethyl)phenol | 442.4 |
| 18.109 (2,6) | 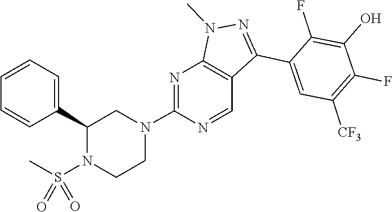 [SMILES: Cn1nc(-c2cc(C(F)(F)F)c(F)c(O)c2F)c2cnc(N3CCN(S(C)(=O)=O)[C@@H](c4ccccc4)C3)nc21] | (S)-2,6-Difluoro-3-(1-methyl-6-(4- (methylsulfony1)-3- phenylpiperazin-1-y1)-1H- pyrazolo[3,4-d]pyrimidin-3-y1)-5- (trifluoromethyl)phenol | 569.5 |
| 18.110 (2,6) | 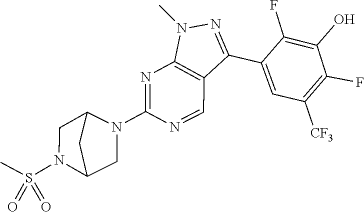 [SMILES: Cn1nc(-c2cc(C(F)(F)F)c(F)c(O)c2F)c2cnc(N3CC4CC3CN4S(C)(=O)=O)nc21] | 2,6-Difluoro-3-(1-methyl-6-(5- (methylsulfony1)-2,5- diazabicyclo[2.2.1]heptan-2-y1)- 1H-pyrazolo[3,4-d]pyrimidin-3-yl)- 5-(trifluoromethyl)phenol | 505.5 |
| 18.111 (2,6) | 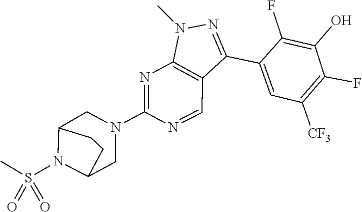 [SMILES: Cn1nc(-c2cc(C(F)(F)F)c(F)c(O)c2F)c2cnc(N3CC4CCC(C3)N4S(C)(=O)=O)nc21] | 2,6-Difluoro-3-(1-methyl-6-(8- (methylsulfony1)-3,8- diazabicyclo[3.2.1]octan-3-yl)-1H- pyrazolo[3,4-d]pyrimidin-3-y1)-5- (trifluoromethyl)phenol | 519.4 |
| 18.112 (2,6) | 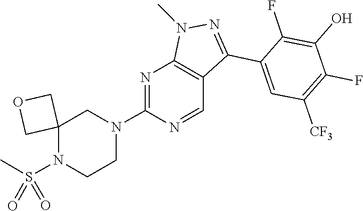 [SMILES: Cn1nc(-c2cc(C(F)(F)F)c(F)c(O)c2F)c2cnc(N3CCN(S(C)(=O)=O)C4(COC4)C3)nc21] | 2,6-Difluoro-3-(1-methyl-6-(5- (methylsulfonyl)-2-oxa-5,8- diazaspiro[3.5]nonan-8-yl)-1H- pyrazolo[3,4-d]pyrimidin-3-y1)-5- (trifluoromethyl)phenol | 535.4 |
| 18.113 (2,6) | 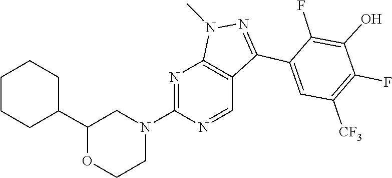 [SMILES: Cn1nc(-c2cc(C(F)(F)F)c(F)c(O)c2F)c2cnc(N3CCOC(C4CCCCC4)C3)nc21] | 3-(6-(2-Cyclohexylmorpholino)-1- methyl-1H-pyrazolo[3,4- d]pyrimidin-3-yl)-2,6-difluoro-5- (trifluoromethyl)phenol | 498.5 |
| 18.114 (2,6) | 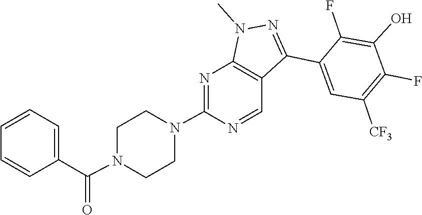 [SMILES: Cn1nc(-c2cc(C(F)(F)F)c(F)c(O)c2F)c2cnc(N3CCN(C(=O)c4ccccc4)CC3)nc21] | (4-(3-(2,4-Difluoro-3-hydroxy-5- (trifluoromethyl)phenyl)-1-methyl- 1H-pyrazolo[3,4-d]pyrimidin-6- y1)piperazin-1- yl)(phenyl)methanone | 519.6 |
| 18.115 (2,6) | 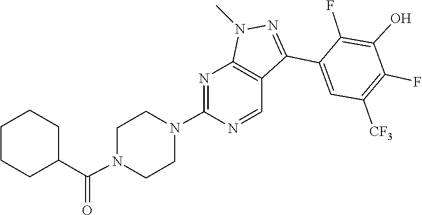 [SMILES: Cn1nc(-c2cc(C(F)(F)F)c(F)c(O)c2F)c2cnc(N3CCN(C(=O)C4CCCCC4)CC3)nc21] | Cyclohexyl(4-(3-(2,4-difluoro-3- hydroxy-5- (trifluoromethyl)phenyl)-1-methyl- 1H-pyrazolo[3,4-d]pyrimidin-6- yl)piperazin-1-yl)methanone | 525.8 |
| 18.116 (2,6) | 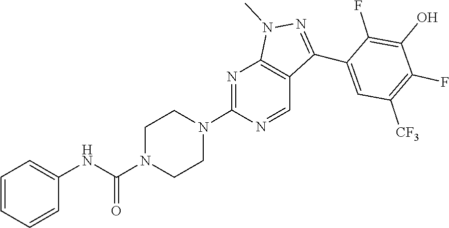 [SMILES: Cn1nc(-c2cc(C(F)(F)F)c(F)c(O)c2F)c2cnc(N3CCN(C(=O)Nc4ccccc4)CC3)nc21] | 4-(3-(2,4-Difluoro-3-hydroxy-5- (trifluoromethyl)phenyl)-1-methyl- 1H-pyrazolo[3,4-d]pyrimidin-6-yl)- N-phenylpiperazine-1-carboxamide | 534.5 |
| 18.117 (2,6) | 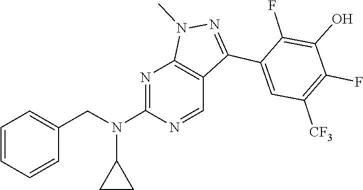 [SMILES: Cn1nc(-c2cc(C(F)(F)F)c(F)c(O)c2F)c2cnc(N(Cc3ccccc3)C3CC3)nc21] | 3-(6-(Benzyl(cyclopropyl)amino)- 1-methyl-1H-pyrazolo[3,4- d]pyrimidin-3-y1)-2,6-difluoro-5- (trifluoromethyl)phenol | 476.4 |
| 18.118 (2,6) | 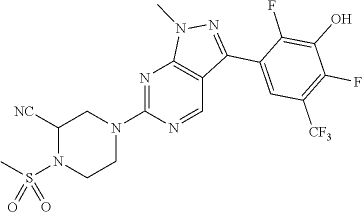 [SMILES: Cn1nc(-c2cc(C(F)(F)F)c(F)c(O)c2F)c2cnc(N3CCN(S(C)(=O)=O)C(C#N)C3)nc21] | 4-(3-(2,4-Difluoro-3-hydroxy-5- (trifluoromethyl)phenyl)-1-methyl- 1H-pyrazolo[3,4-d]pyrimidin-6-yl)- 1-(methylsulfonyl)piperazine-2- carbonitrile | 518.4 |
| 18.119 (2,6) | 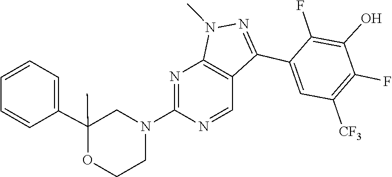 [SMILES: Cn1nc(-c2cc(C(F)(F)F)c(F)c(O)c2F)c2cnc(N3CCOC(C)(c4ccccc4)C3)nc21] | 2,6-Difluoro-3-(1-methyl-6-(2- methyl-2-phenylmorpholino)-1H- pyrazolo[3,4-d]pyrimidin-3-y1)-5- (trifluoromethyl)phenol | 506.5 |
| 18.120 (2,6) | 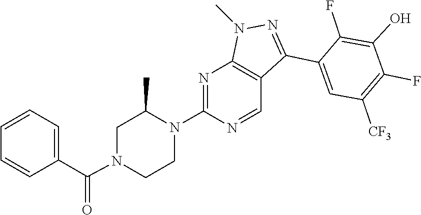 [SMILES: C[C@@H]1CN(C(=O)c2ccccc2)CCN1c1ncc2c(-c3cc(C(F)(F)F)c(F)c(O)c3F)nn(C)c2n1] | (R)-(4-(3-(2,4-Difluoro-3-hydroxy- 5-(trifluoromethyl)phenyl)-1- methyl-1H-pyrazolo[3,4- d]pyrimidin-6-yl)-3- methylpiperazin-1- y1)(phenyl)methanone | 533.8 |
| 18.121 (2,6) | 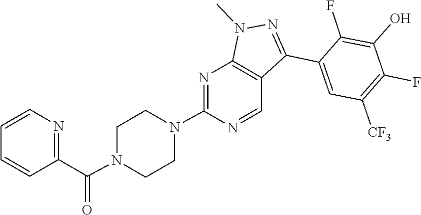 [SMILES: Cn1nc(-c2cc(C(F)(F)F)c(F)c(O)c2F)c2cnc(N3CCN(C(=O)c4ccccn4)CC3)nc21] | (4-(3-(2,4-Difluoro-3-hydroxy-5- (trifluoromethyl)phenyl)-1-methyl- 1H-pyrazolo[3,4-d]pyrimidin-6- yl)piperazin-1-yl)(pyridin-2- yl)methanone | 520.6 |
| 18.122 (2,6) | 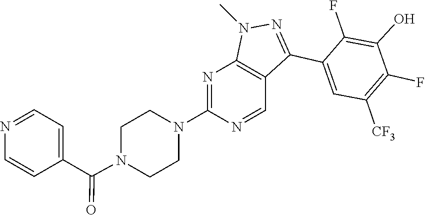 [SMILES: Cn1nc(-c2cc(C(F)(F)F)c(F)c(O)c2F)c2cnc(N3CCN(C(=O)c4ccncc4)CC3)nc21] | (4-(3-(2,4-Difluoro-3-hydroxy-5- (trifluoromethyl)phenyl)-1-methyl- 1H-pyrazolo[3,4-d]pyrimidin-6- y1)piperazin-1-yl)(pyridin-4- yl)methanone | 520.6 |
| 18.123 (2,6) | 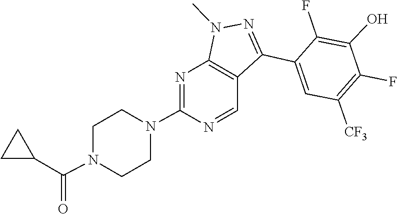 [SMILES: Cn1nc(-c2cc(C(F)(F)F)c(F)c(O)c2F)c2cnc(N3CCN(C(=O)C4CC4)CC3)nc21] | Cyclopropyl(4-(3-(2,4-difluoro-3- hydroxy-5- (trifluoromethyl)phenyl)-1-methyl- 1H-pyrazolo[3,4-d]pyrimidin-6- y1)piperazin-1-yl)methanone | 483.8 |
| 18.124 (2,6) | 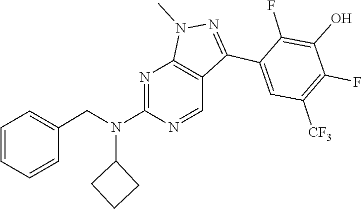 [SMILES: Cn1nc(-c2cc(C(F)(F)F)c(F)c(O)c2F)c2cnc(N(Cc3ccccc3)C3CCC3)nc21] | 3-(6-(Benzyl(cyclobutyl)amino)-1- methyl-1H-pyrazolo[3,4- d]pyrimidin-3-y1)-2,6-difluoro-5- (trifluoromethyl)phenol | 490.6 |
| 18.125 (2,6) | 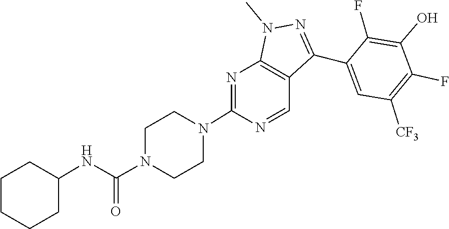 [SMILES: Cn1nc(-c2cc(C(F)(F)F)c(F)c(O)c2F)c2cnc(N3CCN(C(=O)NC4CCCCC4)CC3)nc21] | N-Cyclohexyl-4-(3-(2,4-difluoro-3- hydroxy-5- (trifluoromethyl)phenyl)-1-methyl- 1H-pyrazolo[3,4-d]pyrimidin-6- y1)piperazine-1-carboxamide | 540.7 |
| 18.126 (2,6) | 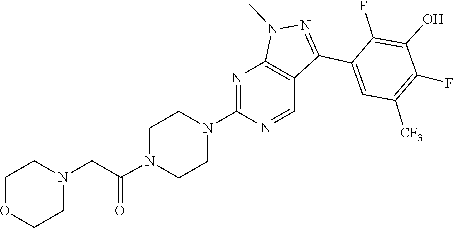 [SMILES: Cn1nc(-c2cc(C(F)(F)F)c(F)c(O)c2F)c2cnc(N3CCN(C(=O)CN4CCOCC4)CC3)nc21] | 1-(4-(3-(2,4-Difluoro-3-hydroxy-5- (trifluoromethyl)phenyl)-1-methyl- 1H-pyrazolo[3,4-d]pyrimidin-6- y1)piperazin-1-yl)-2- morpholinoethan-1-one | 542.6 |
| 18.127 (2,6) | 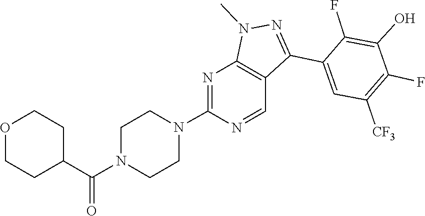 [SMILES: Cn1nc(-c2cc(C(F)(F)F)c(F)c(O)c2F)c2cnc(N3CCN(C(=O)C4CCOCC4)CC3)nc21] | (4-(3-(2,4-Difluoro-3-hydroxy-5- (trifluoromethyl)phenyl)-1-methyl- 1H-pyrazolo[3,4-d]pyrimidin-6- yl)piperazin-1-yl)(tetrahydro-2H- pyran-4-yl)methanone | 527.6 |
| 18.128 (2,6) | 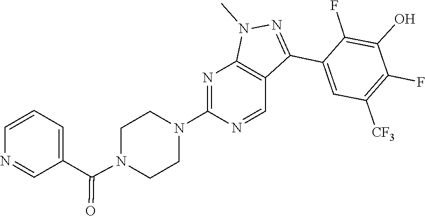 [SMILES: Cn1nc(-c2cc(C(F)(F)F)c(F)c(O)c2F)c2cnc(N3CCN(C(=O)c4cccnc4)CC3)nc21] | (4-(3-(2,4-Difluoro-3-hydroxy-5- (trifluoromethyl)phenyl)-1-methyl- 1H-pyrazolo[3,4-d]pyrimidin-6- yl)piperazin-1-yl)(pyridin-3- yl)methanone | 520.6 |
| 18.129 (2,6) | 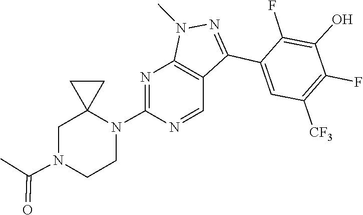 [SMILES: CC(=O)N1CCN(c2ncc3c(-c4cc(C(F)(F)F)c(F)c(O)c4F)nn(C)c3n2)C2(CC2)C1] | 1-(4-(3-(2,4-Difluoro-3-hydroxy-5- (trifluoromethyl)phenyl)-1-methyl- 1H-pyrazolo[3,4-d]pyrimidin-6-yl)- 4,7-diazaspiro[2.5]octan-7- yl)ethan-1-one | 483.6 |
| 18.130 (2,6) | 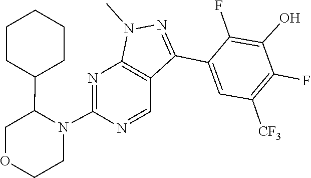 [SMILES: Cn1nc(-c2cc(C(F)(F)F)c(F)c(O)c2F)c2cnc(N3CCOCC3C3CCCCC3)nc21] | 3-(6-(3-Cyclohexylmorpholino)-1- methyl-1H-pyrazolo[3,4- d]pyrimidin-3-y1)-2,6-difluoro-5- (trifluoromethyl)phenol | 498.6 |
| 18.131 (7,8) | 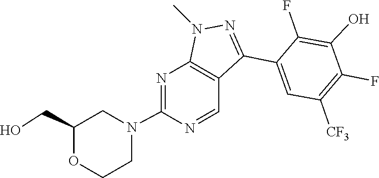 [SMILES: Cn1nc(-c2cc(C(F)(F)F)c(F)c(O)c2F)c2cnc(N3CCO[C@@H](CO)C3)nc21] | (R)-2,6-Difluoro-3-(6-(2- (hydroxymethyl)morpholino)-1- methyl-1H-pyrazolo[3,4- d]pyrimidin-3-y1)-5- (trifluoromethyl)phenol | 446.5 |
| 18.132 (4,9) | 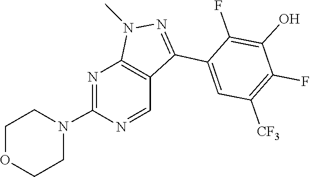 [SMILES: Cn1nc(-c2cc(C(F)(F)F)c(F)c(O)c2F)c2cnc(N3CCOCC3)nc21] | 2,6-Difluoro-3-(1-methyl-6- morpholino-1H-pyrazolo[3,4- d]pyrimidin-3-y1)-5- (trifluoromethyl)phenol | 416.5 |

Alternate conditions used:
1. Boc amine was used;
2. Heated at 110-150° C. in a microwave;
3. Heated at 100° C., 1 h then 125° C., 40 min in a microwave;
4. Also, TFA/CH₂Cl₂ (1:2 or 1:5), rt, 0.5-15 h was needed for full conversion to phenol and/or to remove Boc or THP;
5. Acidified aqueous layer with 1 N HCI during workup to get compound into organic layer;
6. NMP instead of DMA;
7. DIEA, NMP, 60-70° C., 1-3 h;
8. Isolated from acidifying aqueous layer with 1 N HCI in a second extraction with EtOAc;
9. DIEA, NMP, rt, overnight.

The Compounds below were synthesized from Intermediate 18.04 and the appropriate amine using the following sequence: Compound 18 procedure (DIEA, NMP, 70° C., 45 min, microwave), methylation (CH₃I or (iodomethyl)benzene, NaH, DMF, 0° C.-rt, 75 min), and then deprotection (5:1 DCM/TFA, rt, 0.5-1 h).

| | | | |
|---|---|---|---|
| Cmpd | Structure | Name | [M + H]⁺ |

| | | | |
|---|---|---|---|
| 19 (1) | 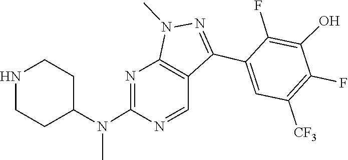 [SMILES: CN(c1ncc2c(-c3cc(C(F)(F)F)c(F)c(O)c3F)nn(C)c2n1)C1CCNCC1] | 2,6-Difluoro-3-(1-methyl-6- (methyl(piperidin-4-yl)amino)-1H- pyrazolo[3,4-d]pyrimidin-3-y1)-5- (trifluoromethyl)phenol | 443.6 |
| 19.01 | 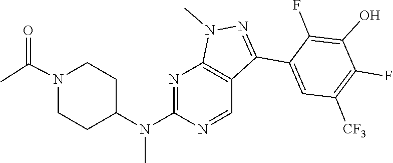 [SMILES: CC(=O)N1CCC(N(C)c2ncc3c(-c4cc(C(F)(F)F)c(F)c(O)c4F)nn(C)c3n2)CC1] | 1-(4-((3-(2,4-Difluoro-3-hydroxy- 5-(trifluoromethyl)phenyl)-1- methyl-1H-pyrazolo[3,4- d]pyrimidin-6- y1)(methyl)amino)piperidin-1- yl)ethan-1-one | 485.6 |
| 19.02 | 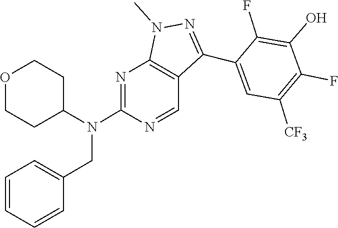 [SMILES: Cn1nc(-c2cc(C(F)(F)F)c(F)c(O)c2F)c2cnc(N(Cc3ccccc3)C3CCOCC3)nc21] | 3-(6-(Benzyl(tetrahydro-2H-pyran- 4-yl)amino)-1-methyl-1H- pyrazolo[3,4-d]pyrimidin-3-y1)-2,6- difluoro-5-(trifluoromethyl)phenol | 520.6 |

Alternate conditions used:
1. Boc amine was used.

The Compounds below were synthesized from Intermediate 18.24, tert-butyl 5,8-diazaspiro[3.5]nonane-5-carboxylate, and the appropriate acylating agent using the following sequence: Compound 18 procedure (DIEA, NMP, 120° C., 2 h), Boc deprotection (4 M HCl in EtOAc, rt, 2 h), acylation (TEA, DCM, 0° C.-rt or 40° C., 2 h-ON), and then Bn deprotection (TFA, 50-70° C., 2 h).

| Cmpd | Structure | Name | [M + H]⁺ |
|---|---|---|---|
| 20 (1) | 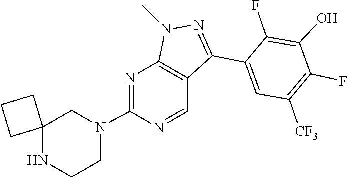 [SMILES: Cn1nc(-c2cc(C(F)(F)F)c(F)c(O)c2F)c2cnc(N3CCNC4(CCC4)C3)nc21] | 2,6-Difluoro-3-(1-methyl-6-(5,8- diazaspiro[3.5]nonan-8-yl)-1H- pyrazolo[3,4-d]pyrimidin-3-y1)-5- (trifluoromethyl)phenol HCl salt | 455.0 |
| 20.01 | 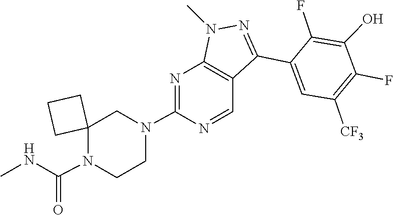 [SMILES: CNC(=O)N1CCN(c2ncc3c(-c4cc(C(F)(F)F)c(F)c(O)c4F)nn(C)c3n2)CC12CCC2] | 8-(3-(2,4-Difluoro-3-hydroxy-5- (trifluoromethyl)phenyl)-1-methyl- 1H-pyrazolo[3,4-d]pyrimidin-6-yl)- N-methyl-5,8- diazaspiro[3.5]nonane-5- carboxamide | 512.1 |
| 20.02 | 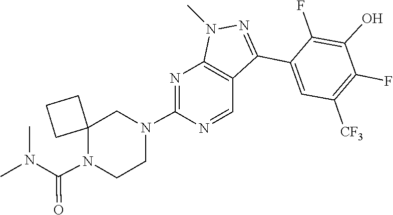 [SMILES: CN(C)C(=O)N1CCN(c2ncc3c(-c4cc(C(F)(F)F)c(F)c(O)c4F)nn(C)c3n2)CC12CCC2] | 8-(3-(2,4-Difluoro-3-hydroxy-5- (trifluoromethyl)phenyl)-1-methyl- 1H-pyrazolo[3,4-d]pyrimidin-6-yl)- N,N-dimethy1-5,8- diazaspiro[3.5]nonane-5- carboxamide | 526.1 |
| 20.03 | 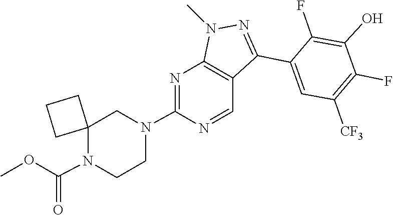 [SMILES: COC(=O)N1CCN(c2ncc3c(-c4cc(C(F)(F)F)c(F)c(O)c4F)nn(C)c3n2)CC12CCC2] | Methyl 8-(3-(2,4-difluoro-3- hydroxy-5- (trifluoromethyl)phenyl)-1-methyl- 1H-pyrazolo[3,4-d]pyrimidin-6-yl)- 5,8-diazaspiro[3.5]nonane-5- carboxylate | 513.0 |

1. Benzyl was partially cleaved in step 1; this compound was isolated after step 2.

### Compound 21

### (R)-4-(3-(2,4-Difluoro-3-hydroxy-5-(trifluoromethyl)phenyl)-1-methyl-1H-pyrazolo[4,3-c]pyridin-6-yl)-3-methyl-N-phenylpiperazine-1-carboxamide

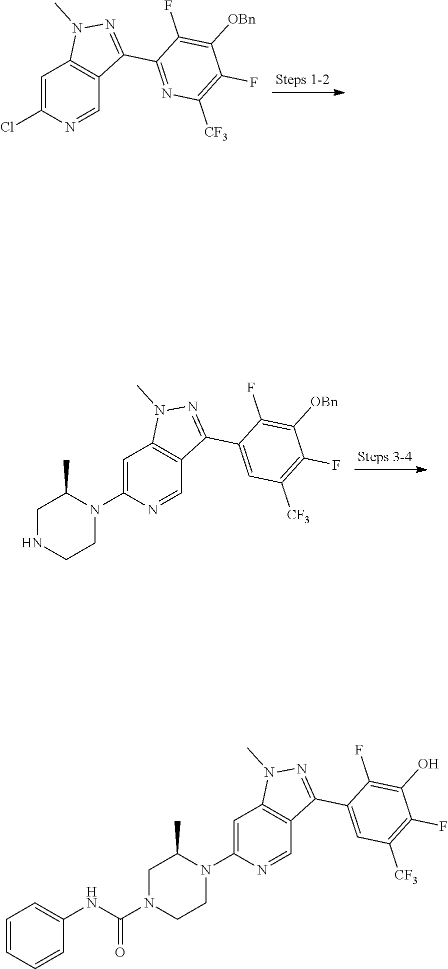 [SMILES: Cn1nc(-c2nc(C(F)(F)F)c(F)c(OCc3ccccc3)c2F)c2cnc(Cl)cc21 ; C[C@@H]1CNCCN1c1cc2c(cn1)c(-c1cc(C(F)(F)F)c(F)c(OCc3ccccc3)c1F)nn2C ; C[C@@H]1CN(C(=O)Nc2ccccc2)CCN1c1cc2c(cn1)c(-c1cc(C(F)(F)F)c(F)c(O)c1F)nn2C]

### Step 1: (R)-tert-Butyl 4-(3-(3-(benzyloxy)-2,4-difluoro-5-(trifluoromethyl)phenyl)-1-methyl-1H-pyrazolo[4,3-c]pyridin-6-yl)-3-methylpiperazine-1-carboxylate

Tris(dibenzylideneacetone)dipalladium(0) (107 mg, 0.116 mmol) and RuPhos (109 mg, 0.233 mmol) were added to a mixture of Intermediate 18.22 (530 mg, 1.17 mmol), NaOtBu (225 mg, 2.34 mmol), and tert-butyl (3R)-3-methylpiperazine-1-carboxylate (234 mg, 1.17 mmol) in dioxane (10 mL). The mixture was degassed and purged with N₂ 3 times, stirred at 80° C. for 2 h, allowed to cool to rt, poured into H₂O (20 mL), and then extracted with EtOAc (3×15 mL). The combined organic layers were washed with brine (2×20 mL), dried (Na₂SO₄), filtered, concentrated, and then purified by silica gel chromatography (petroleum ether/ethyl acetate=10/1) to obtain (R)-tert-butyl 4-(3-(3-(benzyloxy)-2,4-difluoro-5-(trifluoromethyl)phenyl)-1-methyl-1H-pyrazolo[4,3-c]pyridin-6-yl)-3-methylpiperazine-1-carboxylate (320 mg, 44%) as a yellow oil. ¹H NMR (400 MHz, DMSO-d₆): δ 8.78-8.72 (m, 1H), 7.83 (t, 1H), 7.53-7.48 (m, 2H), 7.46-7.37 (m, 3H), 6.75 (d, 1H), 5.42-5.30 (m, 2H), 4.70 (d, 1H), 4.15-3.92 (m, 4H), 3.82 (d, 1H), 3.28-2.90 (m, 3H), 2.52 (d, 1H), 1.44 (s, 9H), 1.04 (d, 3H); LCMS: 618.3 [M+H]⁺.

### Step 2: (R)-3-(3-(Benzyloxy)-2,4-difluoro-5-(trifluoromethyl)phenyl)-1-methyl-6-(2-methylpiperazin-1-yl)-1H-pyrazolo[4,3-c]pyridine

A mixture of tert-butyl (S)-tert-butyl 4-(3-(3-(benzyloxy)-2,4-difluoro-5-(trifluoromethyl)phenyl)-1-methyl-1H-pyrazolo[4,3-c]pyridin-6-yl)-3-methylpiperazine-1-carboxylate (300 mg, 0.485 mmol) in 4 M HCl/EtOAc (5 mL) was stirred at rt for 2 h and then concentrated to obtain (R)-3-(3-(benzyloxy)-2,4-difluoro-5-(trifluoromethyl)phenyl)-1-methyl-6-(2-methylpiperazin-1-yl)-1H-pyrazolo[4,3-c]pyridine HCl salt (310 mg) as a yellow oil. LCMS: 518.2 [M+H]⁺

### Step 3: (R)-4-(3-(3-(Benzyloxy)-2,4-difluoro-5-(trifluoromethyl)phenyl)-1-methyl-1H-pyrazolo[4,3-c]pyridin-6-yl)-3-methyl-N-phenylpiperazine-1-carboxamide

Triethylamine (0.48 mL, 3.48 mmol) and isocyanatobenzene (69 mg, 0.58 mmol) were added to a solution of (R)-3-(3-(benzyloxy)-2,4-difluoro-5-(trifluoromethyl)phenyl)-1-methyl-6-(2-methylpiperazin-1-yl)-1H-pyrazolo[4,3-c]pyridine HCl salt (300 mg, 0.579 mmol) in DCM (5 mL) at 0° C. The mixture was stirred at rt for 2 h, poured into sat. aq. NaHCO₃ (10 mL), and then extracted with DCM (3×10 mL). The combined organic layers were washed with brine (2×20 mL), dried (Na₂SO₄), filtered, concentrated, and then purified by prep-TLC (SiO₂, petroleum ether/EtOAc=1:1) to obtain (R)-4-(3-(3-(benzyloxy)-2,4-difluoro-5-(trifluoromethyl)phenyl)-1-methyl-1H-pyrazolo[4,3-c]pyridin-6-yl)-3-methyl-N-phenylpiperazine-1-carboxamide (180 mg, 49%) as a white solid. ¹H NMR (400 MHz, DMSO-d₆): δ 8.83-8.70 (m, 1H), 8.58 (s, 1H), 7.84 (t, 1H), 7.53-7.47 (m, 4H), 7.46-7.36 (m, 3H), 7.25 (t, 2H), 6.95 (t, 1H), 6.79 (s, 1H), 5.36-5.25 (m, 2H), 4.80-4.66 (m, 1H), 4.22 (d, 1H), 4.09 (d, 2H), 4.03-3.98 (m, 3H), 3.29-3.22 (m, 1H), 3.21-3.13 (m, 1H), 3.13-3.03 (m, 1H), 1.13-1.06 (m, 3H); LCMS: 637.2 [M+H]⁺

### Step 4: (R)-4-(3-(2,4-Difluoro-3-hydroxy-5-(trifluoromethyl)phenyl)-1-methyl-1H-pyrazolo[4,3-c]pyridin-6-yl)-3-methyl-N-phenylpiperazine-1-carboxamide

A mixture of (R)-4-(3-(3-(benzyloxy)-2,4-difluoro-5-(trifluoromethyl)phenyl)-1-methyl-1H-pyrazolo[4,3-c]pyridin-6-yl)-3-methyl-N-phenylpiperazine-1-carboxamide (170 mg, 0.267 mmol) in TFA (2 mL) was stirred at 70° C. for 2 h under N₂, allowed to cool to rt slowly, concentrated, and then purified by prep-HPLC [20-50% water (NH₃H₂O+NH₄HCO₃)-ACN] to obtain (R)-4-(3-(2,4-difluoro-3-hydroxy-5-(trifluoromethyl)phenyl)-1-methyl-1H-pyrazolo[4,3-c]pyridin-6-yl)-3-methyl-N-phenylpiperazine-1-carboxamide (80 mg, 55%) as a white solid. ¹H NMR (400 MHz, DMSO-d₆): δ 8.64 (d, 1H), 8.60 (s, 1H), 7.48 (d, 2H), 7.29-7.21 (m, 2H), 7.15-7.05 (m, 1H), 6.98-6.90 (m, 1H), 6.73 (s, 1H), 4.71 (t, 1H), 4.23 (d, 1H), 4.13-4.02 (m, 2H), 3.98 (s, 3H), 3.19-3.02 (m, 3H), 1.08 (d, 3H); LCMS: 547.1 [M+H]⁺.

The Compounds below were synthesized in a similar manner to that described for Compound 21.

| Cmpd | Structure | Name | [M+H]+ |
|---|---|---|---|
| 21.01 | 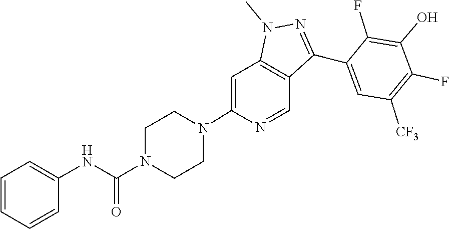 [SMILES: Cn1nc(-c2cc(C(F)(F)F)c(F)c(O)c2F)c2cnc(N3CCN(C(=O)Nc4ccccc4)CC3)cc21] | 4-(3-(2,4-Difluoro-3-hydroxy-5- (trifluoromethyl)phenyl)-1-methyl- 1H-pyrazolo[4,3-c]pyridin-6-y1)-N- phenylpiperazine-1-carboxamide | 533.1 |
| 21.02 | 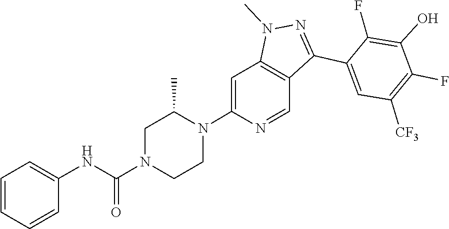 [SMILES: C[C@H]1CN(C(=O)Nc2ccccc2)CCN1c1cc2c(cn1)c(-c1cc(C(F)(F)F)c(F)c(O)c1F)nn2C] | (S)-4-(3-(2,4-Difluoro-3-hydroxy- 5-(trifluoromethyl)phenyl)-1- methyl-1H-pyrazolo[4,3-c]pyridin- 6-y1)-3-methyl-N-phenylpiperazine- 1-carboxamide | 547.1 |
| 21.03 | 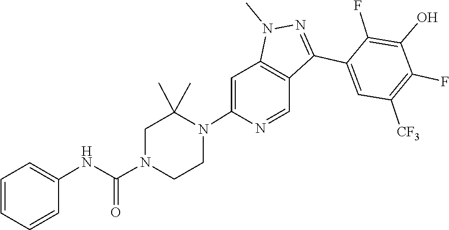 [SMILES: Cn1nc(-c2cc(C(F)(F)F)c(F)c(O)c2F)c2cnc(N3CCN(C(=O)Nc4ccccc4)CC3(C)C)cc21] | 4-(3-(2,4-Difluoro-3-hydroxy-5- (trifluoromethyl)phenyl)-1-methyl- 1H-pyrazolo[4,3-c]pyridin-6-y1)- 3,3-dimethyl-N-phenylpiperazine- 1-carboxamide | 561.1 |
| 21.04 | 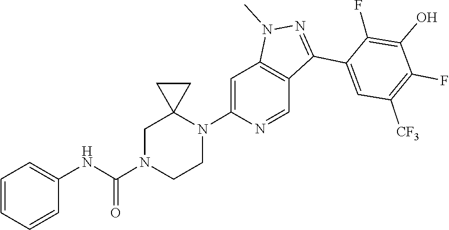 [SMILES: Cn1nc(-c2cc(C(F)(F)F)c(F)c(O)c2F)c2cnc(N3CCN(C(=O)Nc4ccccc4)CC34CC4)cc21] | 4-(3-(2,4-Difluoro-3-hydroxy-5- (trifluoromethyl)phenyl)-1-methyl- 1H-pyrazolo[4,3-c]pyridin-6-y1)-N- phenyl-4,7-diazaspiro[2.5 ]octane-7- carboxamide | 559.1 |
| 21.05 | 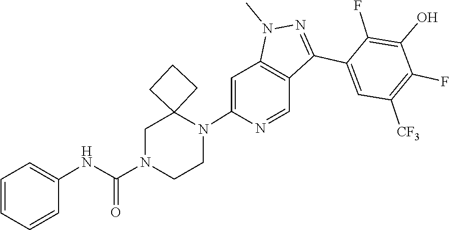 [SMILES: Cn1nc(-c2cc(C(F)(F)F)c(F)c(O)c2F)c2cnc(N3CCN(C(=O)Nc4ccccc4)CC34CCC4)cc21] | 5-(3-(2,4-Difluoro-3-hydroxy-5- (trifluoromethyl)phenyl)-1-methyl- 1H-pyrazolo[4,3-c]pyridin-6-y1)-N- phenyl-5,8-diazaspiro[3.5 ]nonane- 8-carboxamide | 573.1 |
| 21.06 | 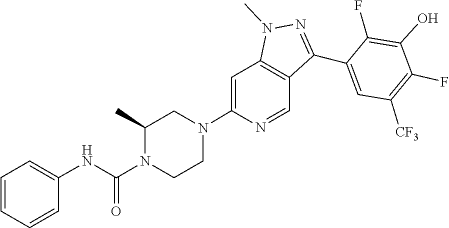 [SMILES: C[C@H]1CN(c2cc3c(cn2)c(-c2cc(C(F)(F)F)c(F)c(O)c2F)nn3C)CCN1C(=O)Nc1ccccc1] | (S)-4-(3-(2,4-Difluoro-3-hydroxy- 5-(trifluoromethyl)phenyl)-1- methyl-1H-pyrazolo[4,3-c]pyridin- 6-y1)-2-methyl-N-phenylpiperazine- 1-carboxamide | 547.0 |
| 21.07 | 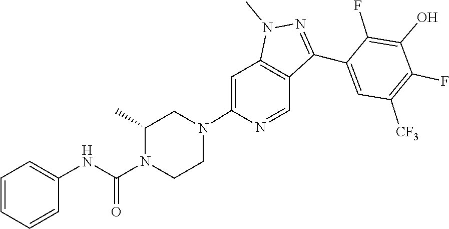 [SMILES: C[C@@H]1CN(c2cc3c(cn2)c(-c2cc(C(F)(F)F)c(F)c(O)c2F)nn3C)CCN1C(=O)Nc1ccccc1] | (R)-4-(3-(2,4-Difluoro-3-hydroxy- 5-(trifluoromethyl)phenyl)-1- methyl-1H-pyrazolo[4,3-c]pyridin- 6-y1)-2-methyl-N-phenylpiperazine- 1-carboxamide | 547.1 |
| 21.08 | 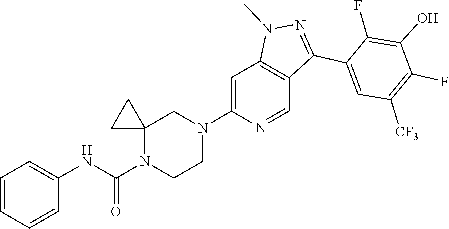 [SMILES: Cn1nc(-c2cc(C(F)(F)F)c(F)c(O)c2F)c2cnc(N3CCN(C(=O)Nc4ccccc4)C4(CC4)C3)cc21] | 7-(3-(2,4-Difluoro-3-hydroxy-5- (trifluoromethyl)phenyl)-1-methyl- 1H-pyrazolo[4,3-c]pyridin-6-y1)-N- phenyl-4,7-diazaspiro[2.5]octane-4- carboxamide | 559.1 |

Alternate conditions used:
Step 1: 70-80° C.; 2-16 h.
Step 1: Cs₂CO₃ instead of NaOtBu.
Step 2: 0.5-2 h.

### Compound 22

### 3,5-Difluoro-2-(1-methyl-6-(methyl(tetrahydro-2H-pyran-4-yl)amino)-1H-pyrazolo[4,3-c]pyridin-3-yl)-6-(trifluoromethyl)pyridin-4-ol

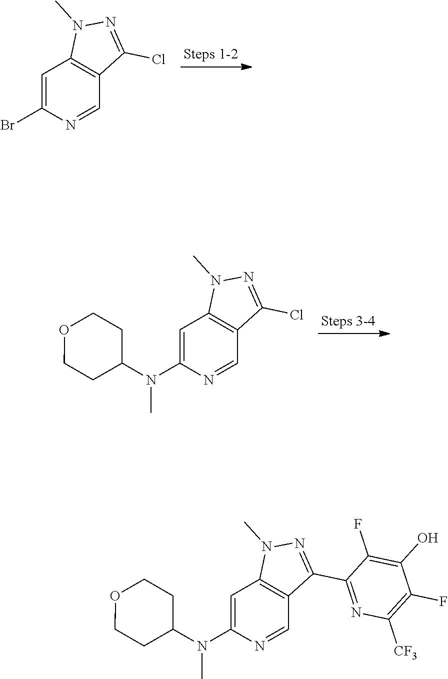 [SMILES: Cn1nc(Cl)c2cnc(Br)cc21 ; CN(c1cc2c(cn1)c(Cl)nn2C)C1CCOCC1 ; CN(c1cc2c(cn1)c(-c1nc(C(F)(F)F)c(F)c(O)c1F)nn2C)C1CCOCC1]

### Step 1: 3-Chloro-1-methyl-N-(tetrahydro-2H-pyran-4-yl)-1H-pyrazolo[4,3-c]pyridin-6-amine

Tetrahydro-2H-pyran-4-amine (191 mg, 1.89 mmol), NaOtBu (483 mg, 5.03 mmol), and XantPhos Pd G4 (121 mg, 0.12 mmol) were added to a solution of Intermediate 17.04 (310 mg, 1.26 mmol) in THF (8 mL) under N₂. The mixture was degassed and purged with N₂ 3 times, stirred at 80° C. for 2 h, allowed to cool to rt, and then partitioned between ethyl acetate (3×20 mL) and H₂O (20 mL). The organic layer was washed with brine (3×20 mL), dried over Na₂SO₄, filtered, concentrated, and then purified by silica gel chromatography (15-40% ethyl acetate in petroleum ether) to give 3-chloro-1-methyl-N-(tetrahydro-2H-pyran-4-yl)-1H-pyrazolo[4,3-c]pyridin-6-amine (420 mg, 76%) as a light green solid. ¹H NMR (400 MHz, DMSO-d₆): δ 8.48 (d, 1H), 6.61 (d, 1H), 6.32 (s, 1H), 3.89-3.86 (m 2H), 3.85-3.76 (m, 4H), 3.47-3.34 (m, 2H), 1.90-1.86 (m, 2H), 1.53-1.40 (m, 2H); LCMS: 267.1 [M+H]⁺.

### Step 2: 3-Chloro-N,1-dimethyl-N-(tetrahydro-2H-pyran-4-yl)-1H-pyrazolo[4,3-c]pyridin-6-amine

Sodium hydride (185 mg, 4.63 mmol, 60% purity) was added to a solution of 3-chloro-1-methyl-N-(tetrahydro-2H-pyran-4-yl)-1H-pyrazolo[4,3-c]pyridin-6-amine (412 mg, 1.54 mmol) in DMF (5 mL) under N₂. The reaction was stirred at rt for 5 min. Iodomethane (0.3 mL, 4.63 mmol) was added. The mixture was stirred at rt for 1 h, quenched with H₂O (20 mL) carefully, and then extracted with ethyl acetate (4×10 mL). The combined organic layers were washed with brine (20 mL), dried over Na₂SO₄, filtered, concentrated, and then purified by silica gel chromatography (20-30% ethyl acetate in petroleum ether) to give 3-chloro-N,1-dimethyl-N-(tetrahydro-2H-pyran-4-yl)-1H-pyrazolo[4,3-c]pyridin-6-amine (350 mg, 80%) as a gray solid. ¹H NMR (400 MHz, DMSO-d₆): δ 8.58 (d, 1H), 6.49 (s, 1H), 4.92 (s, 1H), 3.97-3.93 (m, 2H), 3.87 (s, 3H), 3.50-3.41 (m, 2H), 2.89 (s, 3H), 1.83-1.79 (m, 2H), 1.54-1.50 (m, 2H); LCMS: 281.1 [M+H]⁺.

### Step 3: 3-(4-(Benzyloxy)-3,5-difluoro-6-(trifluoromethyl)pyridin-2-yl)-N,1-dimethyl-N-(tetrahydro-2H-pyran-4-yl)-1H-pyrazolo[4,3-c]pyridin-6-amine

Bis(tri-tert-butylphosphine)palladium(0) (26 mg, 0.050 μmol) and CsF (167 mg, 1.10 mmol) were added to a solution of 3-chloro-N,1-dimethyl-N-(tetrahydro-2H-pyran-4-yl)-1H-pyrazolo[4,3-c]pyridin-6-amine (140 mg, 0.50 mmol) and 4-(benzyloxy)-3,5-difluoro-2-(tributylstannyl)-6-(trifluoromethyl)pyridine (433 mg, 0.75 mmol) in dioxane (8 mL) under N₂. The mixture was degassed twice with vacuum/N₂, stirred at 110° C. for 2 h, and then allowed to cool to rt. The solids were removed by filtration, and the filtrate was concentrated under reduced pressure. The residue was triturated with toluene (2 mL) at 15° C. for 30 min. The solids were filtered, and the filter cake was dried under reduced pressure to give a mixture of 3-(4-(benzyloxy)-3,5-difluoro-6-(trifluoromethyl)pyridin-2-yl)-N,1-dimethyl-N-(tetrahydro-2H-pyran-4-yl)-1H-pyrazolo[4,3-c]pyridin-6-amine and 3,5-difluoro-2-(1-methyl-6-(methyl(tetrahydro-2H-pyran-4-yl)amino)-1H-pyrazolo[4,3-c]pyridin-3-yl)-6-(trifluoromethyl)pyridin-4-ol (120 mg) as a yellow solid. LCMS: 534.2 [M+H]⁺.

### Step 4: 3,5-Difluoro-2-(1-methyl-6-(methyl(tetrahydro-2H-pyran-4-yl)amino)-1H-pyrazolo[4,3-c]pyridin-3-yl)-6-(trifluoromethyl)pyridin-4-ol

A mixture of 3-(4-(benzyloxy)-3,5-difluoro-6-(trifluoromethyl)pyridin-2-yl)-N,1-dimethyl-N-(tetrahydro-2H-pyran-4-yl)-1H-pyrazolo[4,3-c]pyridin-6-amine and 3,5-difluoro-2-(1-methyl-6-(methyl(tetrahydro-2H-pyran-4-yl)amino)-1H-pyrazolo[4,3-c]pyridin-3-yl)-6-(trifluoromethyl)pyridin-4-ol (120 mg) was dissolved in TFA (3 mL), stirred at 50° C. for 1 h, allowed to cool to rt, concentrated under reduced pressure, and then partitioned between ethyl acetate (4×10 mL) and sat. Na₂CO₃ solution (15 mL). The organic layer was washed with brine (15 mL), dried over Na₂SO₄, filtered, concentrated, and then purified by prep-TLC (DCM:MeOH=10:1) to give 3,5-difluoro-2-(1-methyl-6-(methyl(tetrahydro-2H-pyran-4-yl)amino)-1H-pyrazolo[4,3-c]pyridin-3-yl)-6-(trifluoromethyl)pyridin-4-ol (50.3 mg 49%) as a green solid. ¹H NMR (400 MHz, DMSO-d₆): δ 9.17 (s, 1H), 6.46 (s, 1H), 5.01-4.92 (m, 1H), 3.99-3.89 (m, 5H), 3.53-3.42 (m, 2H), 2.89 (s, 3H), 1.82-1.78 (m, 2H), 1.54-1.52 (m, 2H); LCMS: 444.1 [M+H]⁺.

The Compounds below were synthesized in a similar manner to the procedures described herein.

| Cmpd | Structure | Name | [M + H]⁺ |
|---|---|---|---|
| 23 | 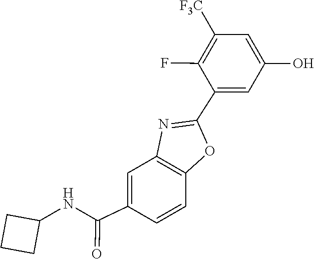 [SMILES: O=C(NC1CCC1)c1ccc2oc(-c3cc(O)cc(C(F)(F)F)c3F)nc2c1] | N-Cyclobuty1-2-(2-fluoro-5- hydroxy-3- (trifluoromethyl)phenyl)benzo[d] oxazole-5-carboxamide | 395.0 |
| 23.01 | 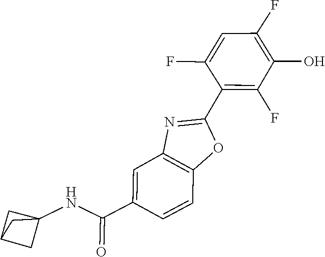 [SMILES: O=C(NC12CC(C1)C2)c1ccc2oc(-c3c(F)cc(F)c(O)c3F)nc2c1] | N-(Bicyclo[1.1.1]pentan-1-y1)-2- (2,4,6-trifluoro-3- hydroxyphenyl)benzo[d]oxazole-5- carboxamide | 375.0 |

### Example A-1: Parenteral Pharmaceutical Composition

To prepare a parenteral pharmaceutical composition suitable for administration by injection (subcutaneous, intravenous), 1-1000 mg of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s), and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 10-500 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound described herein, or a pharmaceutically acceptable salt thereof, is placed into size 4 capsule, or size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl cellulose, propylene glycol, isopropyl myristate and purified alcohol USP. The resulting gel mixture is then incorporated into containers, such as tubes, which are suitable for topical administration.

### Example B-1: HSD17b13 NAD(P)H-Glo Biochemical Assay

### Materials

Recombinant human HSD17B13 enzyme. Substrate: estradiol (Sigma β-Estradiol E8875), 100 mM in DMSO. Cofactor: NAD+ Grade I free acid (Sigma 10127965001), 20 mM in H₂O. Assay buffer final concentration: 20 mM Tris pH7.4 with 0.002% Tween-20 and 0.02% BSA. Assay performed in 384 well solid bottom plate (Corning 3570). Enzymatic activity detected by NAD(P)H-Glo™ Detection System (Promega G9062).

### Compounds

Inhibitor compounds were serially diluted in DMSO and then further diluted in assay buffer to a 10× concentration consisting of 1% DMSO.

### Procedure

HSD17b13 enzyme was diluted in 1× assay buffer to the desired enzyme concentration based on the specific activity of the enzyme lot. 20 uL of diluted enzyme was added to each well along with 2.5 uL of 10× inhibitor solution. Assay plate was incubated at RT for 20 minutes, and then 2.5 uL of a 10× substrate/cofactor mix was added to each well for a final concentration of 50 uM estradiol and 1 mM NAD+. Assay plate was incubated at 37° C. for 3 hours. NAD(P)H-Glo™ Detection System reagents were prepared according to manufacturer's specifications, and 25 uL was added to each well. After incubating for 1 hour at RT, luminescence was measured.

Representative data for exemplary compounds disclosed herein is presented in Table 2.

TABLE 2

| | HSD17β13 |
|---|---|
| Cmpd | IC₅₀ (uM) |
| 1 | +++ |
| 1.01 | + |
| 1.02 | +++ |
| 1.03 | +++ |
| 1.04 | + |
| 1.05 | ++ |
| 1.06 | ++ |
| 1.07 | ++ |
| 1.08 | + |
| 1.09 | +++ |
| 1.10 | +++ |
| 1.11 | ++ |
| 1.12 | +++ |
| 1.13 | +++ |
| 1.14 | +++ |
| 1.15 | ++ |
| 1.16 | ++ |
| 1.17 | ++ |
| 1.18 | + |
| 1.19 | ++ |
| 1.20 | ++ |
| 1.21 | ++ |
| 1.22 | +++ |
| 1.23 | +++ |
| 1.24 | +++ |
| 1.25 | +++ |
| 1.26 | +++ |
| 1.27 | ++ |
| 1.28 | ++ |
| 1.29 | +++ |
| 1.30 | ++ |
| 1.31 | 50% inhibition at 10 uM |
| 2 | ++ |
| 2.01 | +++ |
| 2.02 | 20% inhibition at 30 uM |
| 3 | ++ |
| 3.01 | ++ |
| 3.02 | ++ |
| 3.03 | + |
| 4 | ++ |
| 4.01 | ++ |
| 4.02 | ++ |
| 5 | ++ |
| 5.01 | ++ |
| 5.02 | ++ |
| 6 | +++ |
| 6.01 | ++ |
| 6.02 | +++ |
| 7 | ++ |
| 7.01 | + |
| 7.02 | 40% inhibition at 10 uM |
| 8 | +++ |
| 9 | +++ |
| 9.01 | +++ |
| 9.02 | + |
| 9.03 | +++ |
| 9.04 | +++ |
| 9.05 | ++ |
| 9.06 | +++ |
| 9.07 | +++ |
| 9.08 | +++ |
| 9.09 | ++ |
| 9.10 | +++ |
| 9.11 | +++ |
| 9.12 | +++ |
| 9.13 | +++ |
| 9.14 | +++ |
| 9.15 | +++ |
| 9.16 | +++ |
| 9.17 | +++ |
| 9.18 | +++ |
| 9.19 | +++ |
| 9.20 | +++ |
| 9.21 | +++ |
| 9.22 | 36% inhibition at 10 uM |
| 9.23 | ++ |
| 9.24 | ++ |
| 9.25 | ++ |
| 9.26 | ++ |
| 9.27 | ++ |
| 9.28 | +++ |
| 9.29 | +++ |
| 9.30 | +++ |
| 9.31 | +++ |
| 9.32 | +++ |
| 9.33 | +++ |
| 9.34 | +++ |
| 9.35 | +++ |
| 9.36 | +++ |
| 9.37 | +++ |
| 9.38 | +++ |
| 9.39 | +++ |
| 9.40 | +++ |
| 9.41 | +++ |
| 9.42 | +++ |
| 9.43 | +++ |
| 9.44 | +++ |
| 9.45 | +++ |
| 9.46 | +++ |
| 9.47 | +++ |
| 9.48 | +++ |
| 9.49 | +++ |
| 9.50 | +++ |
| 9.51 | +++ |
| 9.52 | +++ |
| 9.53 | ++ |
| 9.54 | +++ |
| 9.55 | +++ |
| 9.56 | +++ |
| 9.57 | +++ |
| 9.58 | +++ |
| 9.59 | +++ |
| 9.60 | +++ |
| 9.61 | +++ |
| 9.62 | ++ |
| 9.63 | +++ |
| 9.64 | +++ |
| 9.65 | ++ |
| 9.66 | +++ |
| 9.67 | +++ |
| 9.68 | +++ |
| 9.69 | +++ |
| 9.70 | +++ |
| 9.71 | ++ |
| 9.72 | +++ |
| 9.73 | +++ |
| 9.74 | +++ |
| 9.75 | +++ |
| 9.76 | +++ |
| 9.77 | +++ |
| 9.78 | +++ |
| 9.79 | + |
| 9.80 | +++ |
| 9.81 | +++ |
| 9.82 | +++ |
| 9.83 | +++ |
| 9.84 | +++ |
| 9.85 | ++ |
| 9.86 | +++ |
| 9.87 | +++ |
| 9.88 | +++ |
| 9.89 | +++ |
| 9.90 | +++ |
| 9.91 | +++ |
| 9.92 | ++ |
| 9.93 | +++ |
| 9.94 | +++ |
| 9.95 | +++ |
| 9.96 | +++ |
| 9.97 | ++ |
| 9.98 | +++ |
| 9.99 | +++ |
| 9.100 | ++ |
| 9.101 | ++ |
| 9.102 | ++ |
| 9.103 | +++ |
| 9.104 | +++ |
| 9.105 | +++ |
| 9.106 | +++ |
| 9.107 | +++ |
| 9.108 | +++ |
| 9.109 | ++ |
| 9.110 | +++ |
| 9.111 | +++ |
| 9.112 | +++ |
| 9.113 | ++ |
| 9.114 | +++ |
| 9.115 | ++ |
| 9.116 | +++ |
| 9.117 | +++ |
| 9.118 | +++ |
| 9.119 | +++ |
| 9.120 | ++ |
| 9.121 | +++ |
| 9.122 | +++ |
| 9.123 | +++ |
| 9.124 | +++ |
| 9.125 | ++ |
| 9.126 | +++ |
| 9.127 | ++ |
| 9.128 | +++ |
| 9.129 | +++ |
| 9.130 | ++ |
| 9.131 | +++ |
| 9.132 | +++ |
| 9.133 | +++ |
| 9.134 | +++ |
| 9.135 | +++ |
| 9.136 | +++ |
| 9.137 | ++ |
| 9.138 | +++ |
| 9.139 | 25% inhibition at 10 uM |
| 9.140 | 35% inhibition at 10 uM |
| 9.141 | 20% inhibition at 10 uM |
| 10 | ++ |
| 10.01 | ++ |
| 10.02 | +++ |
| 10.03 | ++ |
| 10.04 | ++ |
| 10.05 | +++ |
| 10.06 | ++ |
| 11 | + |
| 11.01 | 41% inhibition at 30 uM |
| 11.02 | ++ |
| 11.03 | +++ |
| 11.04 | +++ |
| 12 | ++ |
| 12.01 | +++ |
| 12.02 | +++ |
| 12.03 | +++ |
| 12.04 | +++ |
| 12.05 | +++ |
| 12.06 | ++ |
| 12.07 | + |
| 12.08 | +++ |
| 12.09 | + |
| 12.10 | <20% inhibition at 10 uM |
| 13 | ++ |
| 13.01 | ++ |
| 14 | ++ |
| 14.01 | +++ |
| 14.02 | +++ |
| 14.03 | ++ |
| 15 | ++ |
| 15.01 | +++ |
| 15.02 | ++ |
| 15.03 | ++ |
| 15.04 | +++ |
| 16 | ++ |
| 17 | ++ |
| 18 | +++ |
| 18.01 | +++ |
| 18.02 | ++ |
| 18.03 | +++ |
| 18.04 | +++ |
| 18.05 | +++ |
| 18.06 | +++ |
| 18.07 | +++ |
| 18.08 | +++ |
| 18.09 | +++ |
| 18.10 | +++ |
| 18.11 | ++ |
| 18.12 | ++ |
| 18.13 | +++ |
| 18.14 | +++ |
| 18.15 | +++ |
| 18.16 | +++ |
| 18.17 | +++ |
| 18.18 | +++ |
| 18.19 | ++ |
| 18.20 | +++ |
| 18.21 | +++ |
| 18.22 | +++ |
| 18.23 | +++ |
| 18.24 | ++ |
| 18.25 | +++ |
| 18.26 | +++ |
| 18.27 | +++ |
| 18.28 | +++ |
| 18.29 | ++ |
| 18.30 | ++ |
| 18.31 | ++ |
| 18.32 | +++ |
| 18.33 | ++ |
| 18.34 | ++ |
| 18.35 | +++ |
| 18.36 | +++ |
| 18.37 | +++ |
| 18.38 | + |
| 18.39 | ++ |
| 18.40 | ++ |
| 18.41 | ++ |
| 18.42 | +++ |
| 18.43 | + |
| 18.44 | +++ |
| 18.45 | ++ |
| 18.46 | +++ |
| 18.47 | +++ |
| 18.48 | +++ |
| 18.49 | +++ |
| 18.50 | + |
| 18.51 | +++ |
| 18.52 | ++ |
| 18.53 | +++ |
| 18.54 | +++ |
| 18.55 | +++ |
| 18.56 | +++ |
| 18.57 | + |
| 18.58 | +++ |
| 18.59 | +++ |
| 18.60 | ++ |
| 18.61 | +++ |
| 18.62 | +++ |
| 18.63 | +++ |
| 18.64 | +++ |
| 18.65 | +++ |
| 18.66 | +++ |
| 18.67 | +++ |
| 18.68 | +++ |
| 18.69 | +++ |
| 18.70 | +++ |
| 18.71 | +++ |
| 18.72 | ++ |
| 18.73 | ++ |
| 18.74 | ++ |
| 18.75 | ++ |
| 18.76 | +++ |
| 18.77 | +++ |
| 18.78 | ++ |
| 18.79 | ++ |
| 18.80 | +++ |
| 18.81 | +++ |
| 18.82 | +++ |
| 18.83 | +++ |
| 18.84 | +++ |
| 18.85 | +++ |
| 18.86 | +++ |
| 18.87 | +++ |
| 18.88 | +++ |
| 18.89 | +++ |
| 18.90 | +++ |
| 18.91 | +++ |
| 18.92 | +++ |
| 18.93 | +++ |
| 18.94 | +++ |
| 18.95 | +++ |
| 18.96 | +++ |
| 18.97 | +++ |
| 18.98 | +++ |
| 18.99 | +++ |
| 18.100 | +++ |
| 18.101 | ++ |
| 18.102 | +++ |
| 18.103 | +++ |
| 18.104 | +++ |
| 18.105 | +++ |
| 18.106 | +++ |
| 18.107 | +++ |
| 18.108 | +++ |
| 18.109 | +++ |
| 18.110 | +++ |
| 18.111 | +++ |
| 18.112 | +++ |
| 18.113 | +++ |
| 18.114 | +++ |
| 18.115 | +++ |
| 18.116 | +++ |
| 18.117 | +++ |
| 18.118 | +++ |
| 18.119 | +++ |
| 18.120 | +++ |
| 18.121 | ++ |
| 18.122 | ++ |
| 18.123 | +++ |
| 18.124 | +++ |
| 18.125 | +++ |
| 18.126 | ++ |
| 18.127 | ++ |
| 18.128 | ++ |
| 18.129 | +++ |
| 18.130 | +++ |
| 18.131 | ++ |
| 18.132 | +++ |
| 19 | ++ |
| 19.01 | +++ |
| 19.02 | +++ |
| 20 | +++ |
| 20.01 | +++ |
| 20.02 | +++ |
| 20.03 | +++ |
| 21 | +++ |
| 21.01 | +++ |
| 21.02 | +++ |
| 21.03 | +++ |
| 21.04 | +++ |
| 21.05 | +++ |
| 21.06 | +++ |
| 21.07 | +++ |
| 21.08 | +++ |
| 22 | ++ |
| 23 | 20% inhibition at 30 uM |
| 23.01 | <20% inhibition at 30 uM |

where ‘+++’ means IC₅₀ ≤ 0.1 uM; where ‘++’ means 0.1 uM < IC₅₀ ≤ 1 uM; where ‘+’ means 1.0 uM < IC₅₀ ≤ 30 uM.

### Example B-2: HSD17b1 NAD(P)H-Glo Biochemical Assay

### Materials

Recombinant human HSD17B1 enzyme. Substrate: testosterone (Sigma T1500), 100 mM in DMSO. Cofactor: NADP disodium salt (Sigma 10128031001), 20 mM in H₂O. Assay buffer final concentration: 20 mM Tris pH7.4 with 0.002% Tween-20 and 0.02% BSA. Assay performed in 384 well solid bottom plate (Corning 3570). Enzymatic activity detected by NAD(P)H-Glo™ Detection System (Promega G9062).

### Compounds

Inhibitor compounds were serially diluted in DMSO and then further diluted in assay buffer to a 10× concentration consisting of 10% DMSO.

### Procedure

HSD17b1 enzyme was diluted in 1× assay buffer to the desired enzyme concentration based on the specific activity of the enzyme lot. 20 uL of diluted enzyme was added to each well along with 2.5 uL of the 10× inhibitor solution. Assay plate was incubated at RT for 20 minutes, and then 2.5 uL of a 10× substrate/cofactor mix was added to each well for a final concentration of 55 uM testosterone and 1 mM NADP. Assay plate was incubated at 37° C. for 1 hour. NAD(P)H-Glo™ Detection System reagents were prepared according to manufacturer's specifications, and 25 uL was added to each well. After incubating for 1 hour at RT, luminescence was measured.

### Example B-3: HSD17b2 NAD(P)H-Glo Biochemical Assay

### Materials and Setup

Recombinant human HSD17B2 enzyme. Substrate: estradiol (Sigma β-Estradiol E8875) 2 mM in DMSO. Cofactor: NAD+ Grade I free acid (Sigma 10127965001), 20 mM in H₂O. Assay buffer final concentration: 20 mM Tris pH7.4 with 0.002% Tween-20 and 0.02% BSA. Assay performed in 384 well solid bottom plate (Corning 3570). Enzymatic activity detected by NAD(P)H-Glo™ Detection System (Promega G9062).

### Compounds

Inhibitor compounds were serially diluted in DMSO and then further diluted in assay buffer to a 10× concentration consisting of 1% DMSO.

### Procedure

HSD17b2 enzyme was diluted in 1× assay buffer to the desired enzyme concentration based on the specific activity of the enzyme lot. 20 uL of diluted enzyme was added to each well along with 2.5 uL of 10× inhibitor solution. Assay plate was incubated at RT for 20 minutes, and then 2.5 uL of 10× substrate/cofactor mix was added to each well for a final assay concentration of 1 uM estradiol and 500 uM NAD+. Assay plate was incubated at RT for 1 hour. NAD(P)H-Glo™ Detection System reagents were prepared according to manufacturer's specifications and 25 uL was added to each well. After incubating for 1 hour at RT, luminescence was measured.

### Example B-4: In Vitro HSD17b13 Cell Based Assay

### Seeding

HEK293 cells were plated at 4,000,000 cells per T75 flask with EMEM (ATCC Cat #30-2003) and 10% FBS (Sigma Cat #F2442) and then incubated at 37° C. in 5% CO₂ for 18 hours.

### Transfection and Plate

After the 18 h incubation, media was replaced with 15 mL of fresh media: EMEM without Phenol Red (Quality Biological Cat #112-212-101), 10% CSS (Sigma Cat #F6765) and GlutaMax (Gibco Cat #35050-061). In a polypropylene tube, 20 ug pCMV6 HSD17B13 (Origene Cat #RC213132) was diluted in OptiMEM (Life Technologies, Cat #31985-062) to 2 mL. 60 uL of transfection reagent (X-tremeGENE HP Roche, Cat #06 366 236 001) was added, and the tube was vortexed and incubated at room temperature for 20 minutes. The transfection reagent/DNA mixture was added to the cells in the T75 flask, and the cells were incubated at 37° C. in 5% CO₂for 18 hours. The next day, the cells were resuspended in EMEM media with 10% CSS and plated in a 96 well plate at 80,000 cells/well, 100 uL/well. Cells were incubated at 37° C. in 5% CO₂for 18 hours.

### Test Compounds

Compounds were serially diluted in DMSO (1000× final concentration) and then further diluted in EMEM media with 10% CSS to a 20× final concentration. 10 uL of the 20× compound mix was added to each well of transfected cells, and the cells were incubated at 37° C. in 5% CO₂ for 30 minutes. 100 uL of EMEM media with 100 uM estradiol (Sigma cat #E8875) was added to each well, and the cells were incubated for 4 hours at 37° C. in 5% CO₂. The cell media was collected and examined for estradiol and estrone concentrations by LCMS.

### Example B-5: In Vitro HSD17b11 Cell Based Assay

### Seeding

HEK293 cells were plated at 4,000,000 cells per T75 flask with EMEM (ATCC Cat #30-2003) and 10% FBS (Sigma Cat #F2442) and then incubated at 37° C. in 5% CO₂ for 18 hours.

### Transfection and Plate

After the 18 h incubation, the media was replaced with 15 mL of fresh media: EMEM without Phenol Red (Quality Biological Cat #112-212-101), 10% CSS (Sigma Cat #F6765) and GlutaMax (Gibco Cat #35050-061). In a polypropylene tube, 20 ug pCMV6 HSD17B11 (Origene Cat #RC205941) was diluted in OptiMEM (Life Technologies, Cat #31985-062) to 2 mL. 60 uL of transfection reagent (X-tremeGENEHP Roche, Cat #06 366 236 001) was added, and the tube was vortexed and incubated at room temperature for 20 minutes. The transfection reagent/DNA mixture was added to the cells in the T75 flask, and the cells were incubated at 37° C. in 5% CO₂for 18 hours. The next day, the transfected cells were resuspended in EMEM media with 10% CSS and plated in a 96 well plate at 80,000 cells/well, 100 uL/well. Cells were incubated at 37° C. in 5% CO₂for 18 hours.

### Test Compounds

Compounds were serially diluted in DMSO (1000× final concentration) and then further diluted in EMEM media with 10% CSS to a 20× final concentration. 10 uL of the 20× compound mix was added to each well of the transfected cells, and the cells were incubated at 37° C. in 5% CO₂for 30 minutes. 100 uL of EMEM media with 60 uM of estradiol (Sigma cat #E8875) was added, and the cells were incubated for 4 hours at 37° C. in 5% CO₂. The cell media was examined for estradiol and estrone concentrations by LCMS.

### Example B-6: NASH Activity Study (AMLN Model)

NASH is induced in male C57BL/6 mice by diet-induction with AMLN diet (DIO-NASH) (D09100301, Research Diet, USA) (40% fat (18% trans-fat), 40% carbohydrates (20% fructose) and 2% cholesterol). The animals are kept on the diet for 29 weeks. After 26 weeks of diet induction, liver biopsies are performed for base line histological assessment of disease progression (hepatosteatosis and fibrosis), stratified and randomized into treatment groups according to liver fibrosis stage, steatosis score, and body weight. Three weeks after biopsy the mice are stratified into treatment groups and dosed daily by oral gavage with an HSD17B13 inhibitor for 8 weeks. At the end of the study liver biopsies are performed to assess hepatic steatosis and fibrosis by examining tissue sections stained with H&E and Sirius Red, respectively. Total collagen content in the liver is measured by colorimetric determination of hydroxyproline residues by acid hydrolysis of collagen. Triglycerides and total cholesterol content in liver homogenates are measured in single determinations using autoanalyzer Cobas C-111 with commercial kit (Roche Diagnostics, Germany) according to manufacturer's instructions.

### Example B-7: CCl₄ Fibrosis Model

Fibrosis is induced in C57BL/6 male mice by bi-weekly oral administration of CCl₄. CCl₄ is formulated 1:4 in oil and is oral dosed at a final concentration of 0.5 ul/g mouse. After 2-4 weeks of fibrosis induction the compounds is administered daily by oral gavage for 2-8 weeks of treatment while continuing CCl₄ administration. At study termination livers are formalin fixed and stained with H&E or Sirius Red stain for histopathological evaluation of inflammation and fibrosis. Total collagen content is measured by colorimetric determination of hydroxyproline residues by acid hydrolysis of collagen. Collagen gene induction is measured by qPCR analysis of Col1a1 and Col3a1 mRNA. Serum alanine aminotransferase (ALT) and aspartate aminotransferase (AST) are measured by a clinical chemistry analyzer.

### Example B-8: Mouse PK Study

The plasma pharmacokinetics of any one of the compounds disclosed herein as a test article is measured following a single bolus intravenous and oral administration to mice (CD-1, C57BL, and diet induced obesity mice). Test article is formulated for intravenous administration in a vehicle solution of DMSO, PEG400, hydroxypropyl-β-cyclodextrin (HPβCD) and is administered (for example at a dose volume of 3 mL/kg) at selected dose levels. An oral dosing formulation is prepared in appropriate oral dosing vehicles (vegetable oils, PEG400, Solutol, citrate buffer, or carboxymethyl cellulose) and is administered at a dose volume of 5˜10 mL/kg at selected dose levels. Blood samples (approximately 0.15 mL) are collected by cheek pouch method at pre-determined time intervals post intravenous or oral doses into tubes containing EDTA. Plasma is isolated by centrifugation of blood at 10,000 g for 5 minutes, and aliquots are transferred into a 96-well plate and stored at −60° C. or below until analysis.

Calibration standards of test article are prepared by diluting DMSO stock solution with DMSO in a concentration range. Aliquots of calibration standards in DMSO are combined with plasma from naïve mouse so that the final concentrations of calibration standards in plasma are 10-fold lower than the calibration standards in DMSO. PK plasma samples are combined with blank DMSO to match the matrix. The calibration standards and PK samples are combined with ice-cold acetonitrile containing an analytical internal standard and centrifuged at 1850 g for 30 minutes at 4° C. The supernatant fractions are analyzed by LC/MS/MS and quantitated against the calibration curve. Pharmacokinetic parameters (area under the curve (AUC), Cₘₐₓ, Tₘₐₓ, elimination half-life (T_{1/2}), clearance (CL), steady state volume of distribution (V_{dss}), and mean residence time (MRT)) are calculated via non-compartmental analysis using Microsoft Excel (version 2013).

### Example B-9: Mouse CDA-HFD NASH Model

A NASH phenotype with mild fibrosis can be induced in C57BL/6 mice by feeding a choline-deficient diet with 0.1% methionine and 60% kcal fat (Research Diet A06071302) for 4-12 weeks. After 4-6 weeks of diet induction compounds can be administered daily by oral gavage for 4-8 weeks of treatment while continuing CDA-HFD feeding. At study termination livers can be formalin fixed and stained with H&E and Sirius Red stain histopathological evaluation of steatosis, inflammation, and fibrosis. Total collagen content can be measured by colorimetric determination of hydroxyproline residues by acid hydrolysis of collagen. Collagen gene induction can be measured by qPCR analysis of Col1a1 or Col3a1. Serum alanine aminotransferase (ALT) and aspartate aminotransferase (AST) can be measured by a clinical chemistry analyzer.

What is claimed is:

1. A compound of Formula (I″), or a pharmaceutically acceptable salt or solvate thereof: 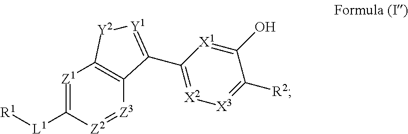
wherein:
X¹, X², and X³ are each independently CR³ or N;
Y¹ is CR⁴ or N;
Y² is N(R⁹), O, or C(R⁴)₂;
Z¹, Z², and Z³ are each independently CR⁵ or N;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
a) C₃₋₁₀cycloalkyl and C₂₋₉heterocycloalkyl, wherein C₃₋₁₀cycloalkyl and C₂₋₉heterocycloalkyl are optionally substituted with one, two, or three R⁶; and
b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R³, each R⁴, and each R⁵ are each independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R⁶ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁷ are each independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹⁴, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
R⁹ is selected from H, C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
each R¹⁴ is independently selected C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, C₁₋₆alkyl, C₁₋₆ haloalkyl, C₁₋₆alkoxy, C₃₋₆cycloalkyl, C₂₋₉heterocycloalkyl, and C₁₋₉heteroaryl.

2. A compound of Formula (II″), or a pharmaceutically acceptable salt or solvate thereof: 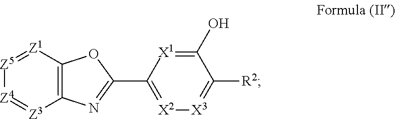
wherein:
X¹, X², and X³ are each independently CR³ or N;
Z¹ and Z³ are each independently CR⁵ or N;
Z⁴ and Z⁵ are each independently CR⁵, CR⁸, or N, wherein one of Z⁴ and Z⁵ is CR⁸;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
a) C₃₋₁₀cycloalkyl and C₂₋₉heterocycloalkyl, wherein C₃₋₁₀cycloalkyl and C₂₋₉heterocycloalkyl are optionally substituted with one, two, or three R⁶; and
b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R³ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁵ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
each R⁷ are each independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —N(R¹⁰)(R¹¹), and —C(O)OR¹⁰;
R⁸ is -L¹-R¹;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

3. A compound of Formula (I′) or Formula (II′), or a pharmaceutically acceptable salt or solvate thereof: 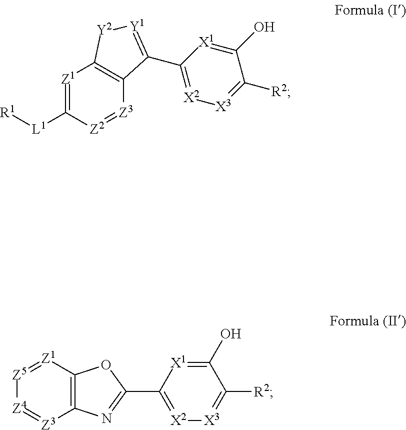
wherein:
X¹, X², and X³ are each independently CR³ or N;
Y¹ is CR⁴ or N;
Y² is N(R⁹), O, or C(R⁴)₂;
Z¹, Z², and Z³ are each independently CR⁵ or N;
Z⁴ and Z⁵ are each independently CR⁵, CR⁸, or N, wherein one of Z⁴ and Z⁵ is CR⁸;
L¹ is selected from a bond, —O—, —N(R¹⁰)—C(O)—, —S(O)₂—, —C(O)N(R¹⁰)—, —N(R¹⁰)C(O)—, —C(R¹⁰)(R¹¹)N(R¹⁰)—, and —N(R¹⁰)C(R¹⁰)(R¹¹)—;
R¹ is selected from:
a) C₃₋₈cycloalkyl and C₂₋₉heterocycloalkyl, wherein C₃₋₈cycloalkyl and C₂₋₉heterocycloalkyl are optionally substituted with one, two, or three R⁶; and
b) C₁₋₉heteroaryl substituted with one, two, or three R⁷;
R² is selected from H, halogen, —CN, C₁₋₆alkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R³, each R⁴, and each R⁵ are each independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R⁶ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁷ is independently selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹⁴, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁸ is -L¹-R¹;
R⁹ is selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆ alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
each R¹⁴ is independently selected C₁₋₆alkyl, C₂₋₆alkenyl, C₂₋₆alkynyl, C₃₋₆cycloalkyl, C₂₋₉heterocycloalkyl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, C₁₋₆alkyl, C₁₋₆ haloalkyl, C₁₋₆alkoxy, C₃₋₆cycloalkyl, C₂₋₉heterocycloalkyl, and C₁₋₉heteroaryl.

4. The compound of claim 3, or a pharmaceutically acceptable salt or solvate thereof, having the structure of Formula (I′):

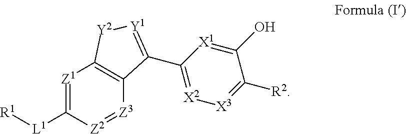

5. The compound of claim 1 or claim 4, or a pharmaceutically acceptable salt or solvate thereof, wherein Y¹ is N.

6. The compound of any one of claims 1 and 3-5, or a pharmaceutically acceptable salt or solvate thereof, wherein Y² is N(R⁹).

7. The compound of any one of claims 1 and 3-6, or a pharmaceutically acceptable salt or solvate thereof, wherein R⁹ is selected from H and C₁₋₆alkyl.

8. The compound of claim 7, or a pharmaceutically acceptable salt or solvate thereof, wherein R⁹ is H.

9. The compound of claim 7, or a pharmaceutically acceptable salt or solvate thereof, wherein R⁹ is C₁₋₆alkyl.

10. The compound of any one of claims 1 and 3-9, or a pharmaceutically acceptable salt or solvate thereof, wherein X¹, X², and X³ are CR³.

11. The compound of any one of claims 1 and 3-10, or a pharmaceutically acceptable salt or solvate thereof, having the structure of Formula (Ia′):

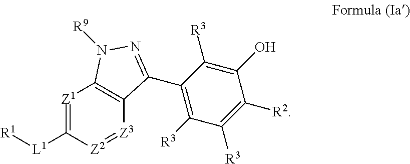

12. The compound of any one of claims 1 and 3-11, or a pharmaceutically acceptable salt or solvate thereof, wherein Z² is CR⁵.

13. The compound of any one of claims 1 and 3-12, or a pharmaceutically acceptable salt or solvate thereof, wherein Z² is N.

14. The compound of claim 3, or a pharmaceutically acceptable salt or solvate thereof, having the structure of Formula (II′):

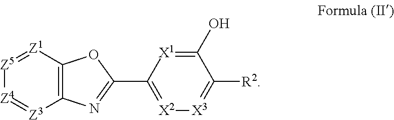

15. The compound of claim 2 or claim 14, or a pharmaceutically acceptable salt or solvate thereof, wherein X¹, X², and X³ are CR³.

16. The compound of any one of claims 2, 14, and 15, or a pharmaceutically acceptable salt or solvate thereof, wherein Z⁵ is CR⁸; and Z⁴ is CR⁵ or N.

17. The compound of claim 2 or claim 16, or a pharmaceutically acceptable salt or solvate thereof, having the structure of Formula (IIa′):

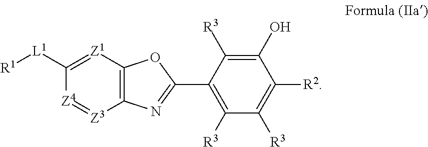

18. The compound of any one of claims 2 and 14-17, or a pharmaceutically acceptable salt or solvate thereof, wherein Z⁴ is CR⁵.

19. The compound of any one of claims 2 and 14-17, or a pharmaceutically acceptable salt or solvate thereof, wherein Z⁴ is N.

20. The compound of claim 14 or claim 15, or a pharmaceutically acceptable salt or solvate thereof, wherein Z⁴ is CR⁸; and Z⁵ is CR⁵ or N.

21. The compound of claim 2 or claim 20, or a pharmaceutically acceptable salt or solvate thereof, having the structure of Formula (IIb′):

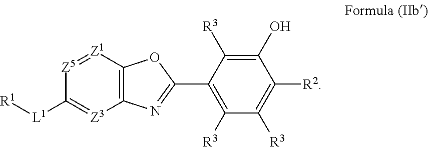

22. The compound of claim 20 or claim 21, or a pharmaceutically acceptable salt or solvate thereof, wherein Z⁵ is CR⁵.

23. The compound of claim 20 or claim 21, or a pharmaceutically acceptable salt or solvate thereof, wherein Z⁵ is N.

24. The compound of any one of claims 1-23, or a pharmaceutically acceptable salt or solvate thereof, wherein L¹ is a bond.

25. The compound of any one of claims 1-23, or a pharmaceutically acceptable salt or solvate thereof, wherein L¹ is —N(R¹⁰)C(O)—.

26. The compound of any one of claims 1-23, or a pharmaceutically acceptable salt or solvate thereof, wherein L¹ is —C(O)N(R¹⁰)—.

27. The compound of any one of claims 1-23, or a pharmaceutically acceptable salt or solvate thereof, wherein L¹ is —N(R¹⁰)—.

28. The compound of any one of claims 1-27, or a pharmaceutically acceptable salt or solvate thereof, wherein Z¹ and Z³ are CR⁵.

29. The compound of any one of claims 1-27, or a pharmaceutically acceptable salt or solvate thereof, wherein Z¹ is N; and Z³ are CR⁵.

30. The compound of any one of claims 1-27, or a pharmaceutically acceptable salt or solvate thereof, wherein Z³ is N; and Z¹ is CR⁵.

31. The compound of any one of claims 1-30, or a pharmaceutically acceptable salt or solvate thereof, wherein R¹ is C₂₋₉heterocycloalkyl optionally substituted with one, two, or three R⁶.

32. The compound of any one of claims 1-31, or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl, wherein piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl are optionally substituted with one, two, or three R⁶.

33. The compound of any one of claims 1-32, or a pharmaceutically acceptable salt or solvate thereof, wherein R¹ is

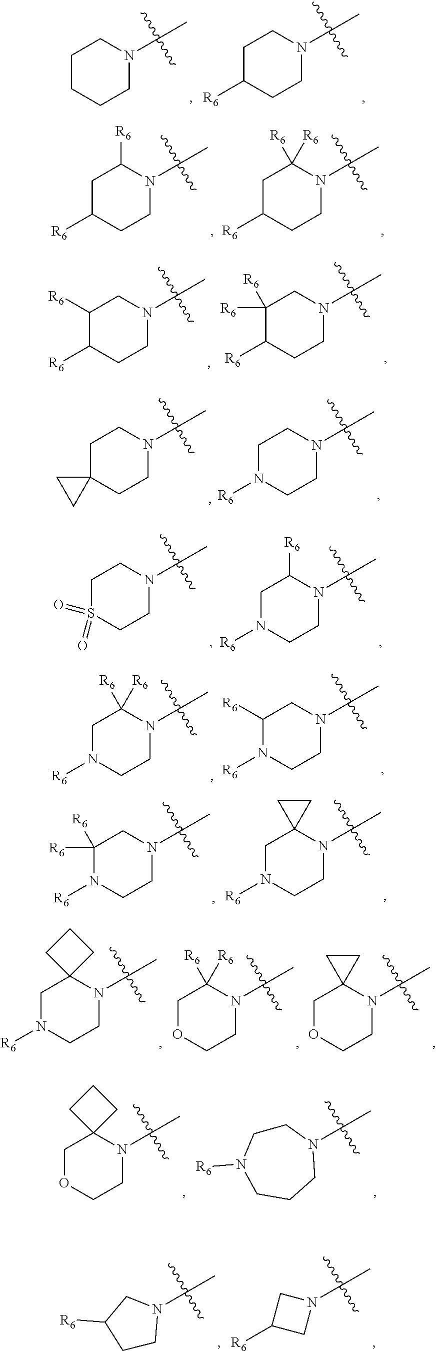

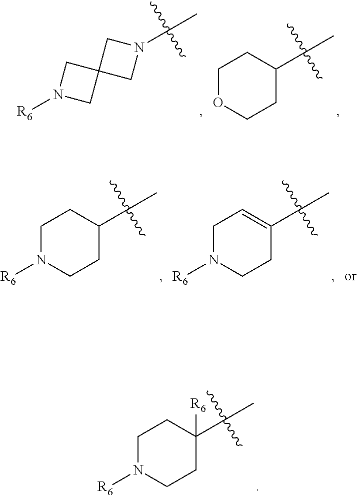

34. The compound of any one of claims 1-33, or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—.

35. The compound of any one of claims 1-34, or a pharmaceutically acceptable salt or solvate thereof, wherein R¹ is

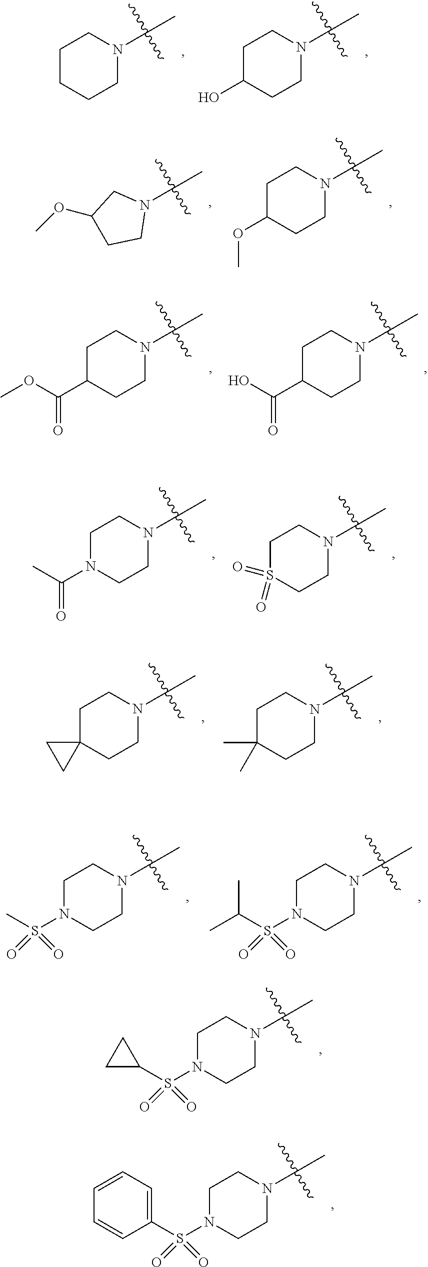

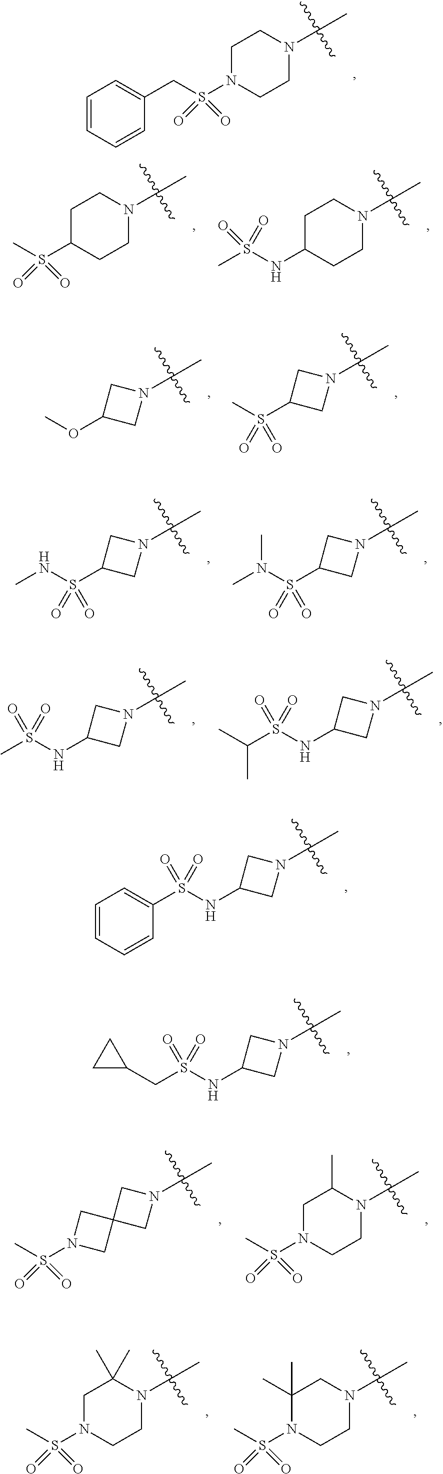

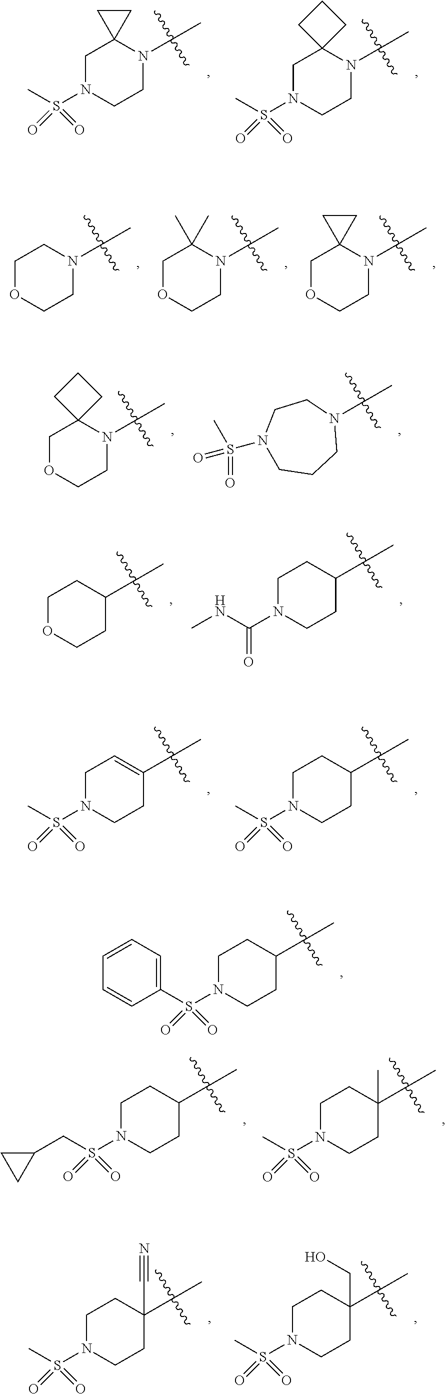

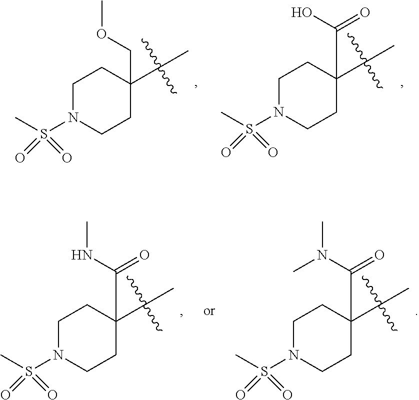 [SMILES: *C1(COC)CCN(S(C)(=O)=O)CC1 ; *C1(C(=O)O)CCN(S(C)(=O)=O)CC1 ; *C1(C(=O)NC)CCN(S(C)(=O)=O)CC1 ; *C1(C(=O)N(C)C)CCN(S(C)(=O)=O)CC1]

36. The compound of any one of claims 1-35, or a pharmaceutically acceptable salt or solvate thereof, wherein R¹ is

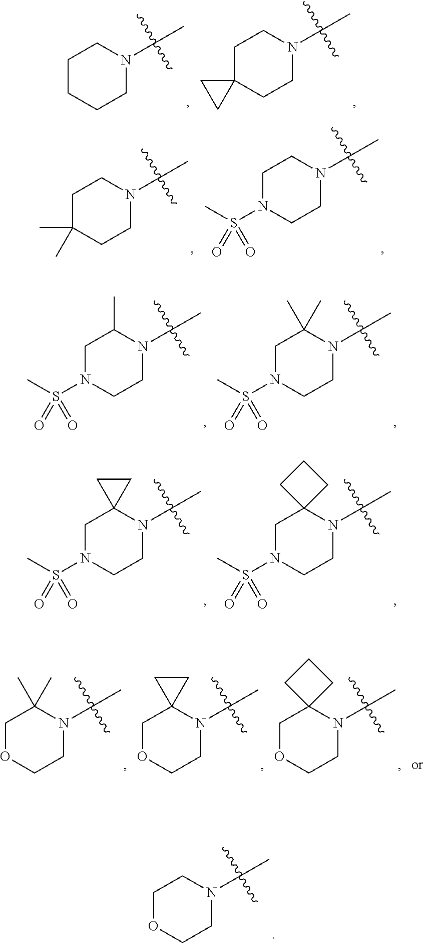

37. The compound of any one of claims 1-30, or a pharmaceutically acceptable salt or solvate thereof, wherein R¹ is C₃₋₈cycloalkyl optionally substituted with one, two, or three R⁶.

38. The compound of claim 37, or a pharmaceutically acceptable salt or solvate thereof, wherein R¹ is

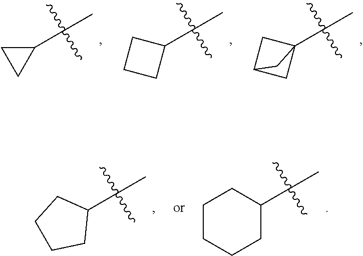

39. The compound of any one of claims 1-30, or a pharmaceutically acceptable salt or solvate thereof, wherein R¹ is C₁₋₉heteroaryl substituted with one, two, or three R⁷.

40. The compound of claim 39, or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷.

41. The compound of claim 39 or claim 40, or a pharmaceutically acceptable salt or solvate thereof, wherein each R¹ is

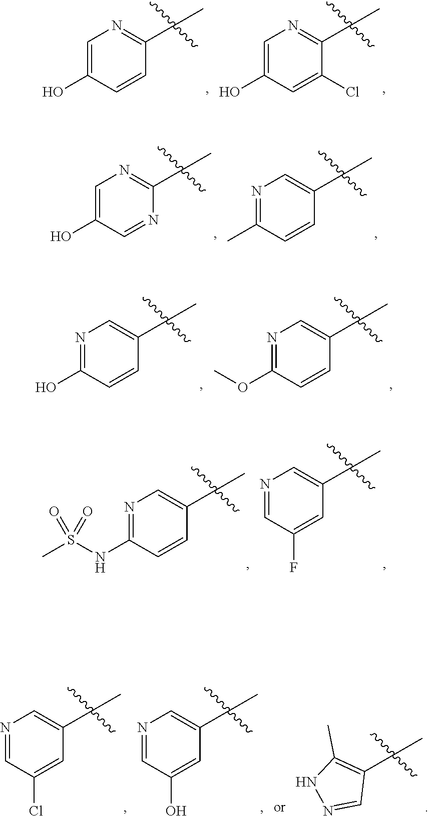

42. The compound of any one of claims 1-41, or a pharmaceutically acceptable salt or solvate thereof, wherein each R⁵ is independently selected from H, halogen, C₁₋₆alkyl, and —OR¹⁰.

43. The compound of any one of claims 1-42, or a pharmaceutically acceptable salt or solvate thereof, wherein each R⁵ is H.

44. The compound of any one of claims 1-43, or a pharmaceutically acceptable salt or solvate thereof, wherein each R³ is independently selected from H, halogen, C₁₋₆alkyl, C₁₋₆haloalkyl, and —OR¹⁰.

45. The compound of any one of claims 1-44, or a pharmaceutically acceptable salt or solvate thereof, wherein R² is H.

46. The compound of any one of claims 1-44, or a pharmaceutically acceptable salt or solvate thereof, wherein R² is halogen.

47. A compound selected from: 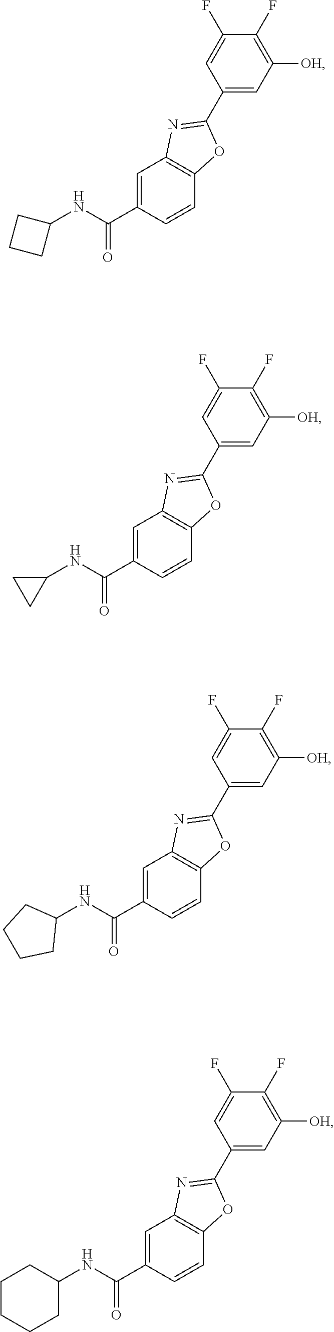 [SMILES: O=C(NC1CCC1)c1ccc2oc(-c3cc(O)c(F)c(F)c3)nc2c1 ; O=C(NC1CC1)c1ccc2oc(-c3cc(O)c(F)c(F)c3)nc2c1 ; O=C(NC1CCCC1)c1ccc2oc(-c3cc(O)c(F)c(F)c3)nc2c1 ; O=C(NC1CCCCC1)c1ccc2oc(-c3cc(O)c(F)c(F)c3)nc2c1] 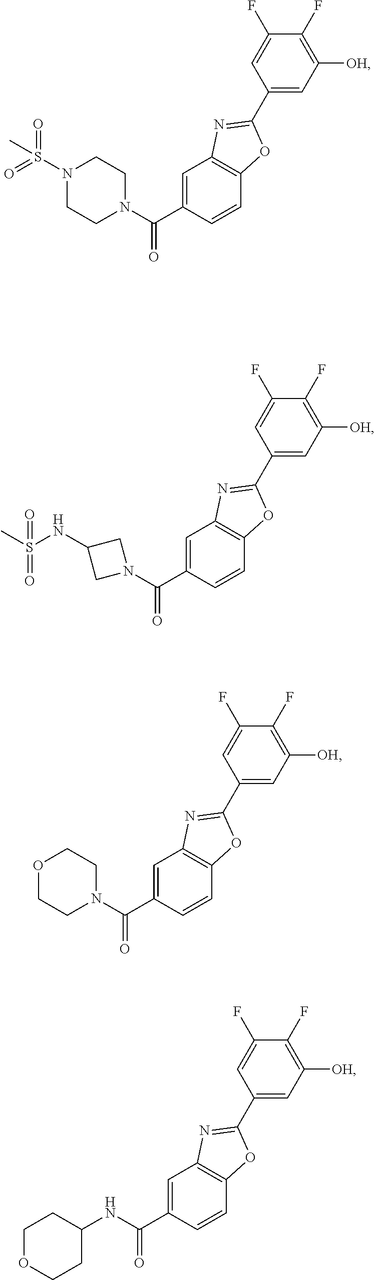 [SMILES: CS(=O)(=O)N1CCN(C(=O)c2ccc3oc(-c4cc(O)c(F)c(F)c4)nc3c2)CC1 ; CS(=O)(=O)NC1CN(C(=O)c2ccc3oc(-c4cc(O)c(F)c(F)c4)nc3c2)C1 ; O=C(c1ccc2oc(-c3cc(O)c(F)c(F)c3)nc2c1)N1CCOCC1 ; O=C(NC1CCOCC1)c1ccc2oc(-c3cc(O)c(F)c(F)c3)nc2c1] 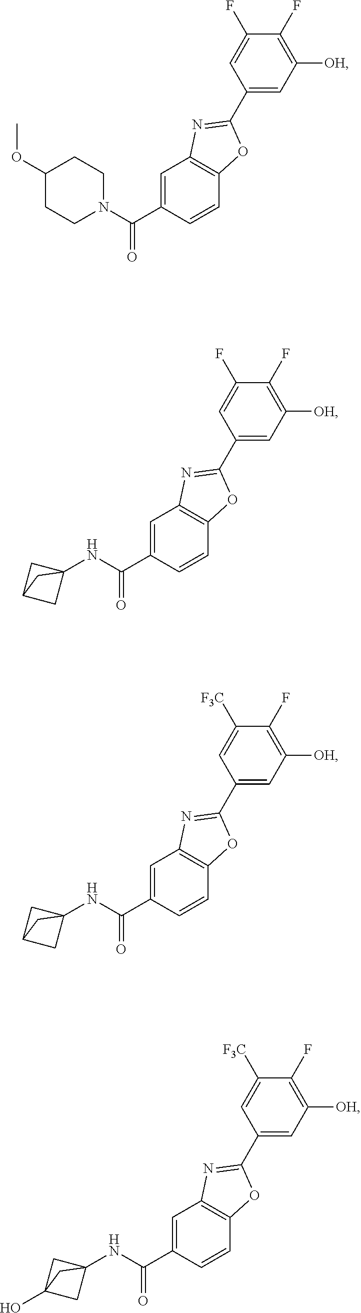 [SMILES: COC1CCN(C(=O)c2ccc3oc(-c4cc(O)c(F)c(F)c4)nc3c2)CC1 ; O=C(NC12CC(C1)C2)c1ccc2oc(-c3cc(O)c(F)c(F)c3)nc2c1 ; O=C(NC12CC(C1)C2)c1ccc2oc(-c3cc(O)c(F)c(C(F)(F)F)c3)nc2c1 ; O=C(NC12CC(O)(C1)C2)c1ccc2oc(-c3cc(O)c(F)c(C(F)(F)F)c3)nc2c1] 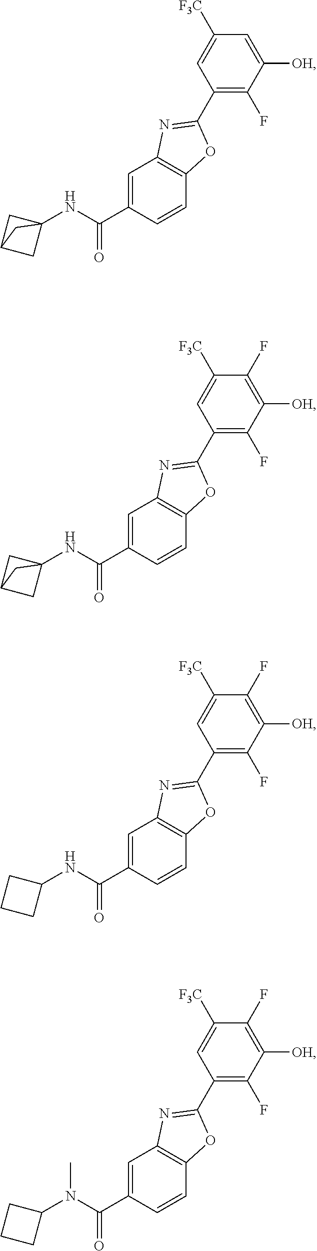 [SMILES: O=C(NC12CC(C1)C2)c1ccc2oc(-c3cc(C(F)(F)F)cc(O)c3F)nc2c1 ; O=C(NC12CC(C1)C2)c1ccc2oc(-c3cc(C(F)(F)F)c(F)c(O)c3F)nc2c1 ; O=C(NC1CCC1)c1ccc2oc(-c3cc(C(F)(F)F)c(F)c(O)c3F)nc2c1 ; CN(C(=O)c1ccc2oc(-c3cc(C(F)(F)F)c(F)c(O)c3F)nc2c1)C1CCC1] 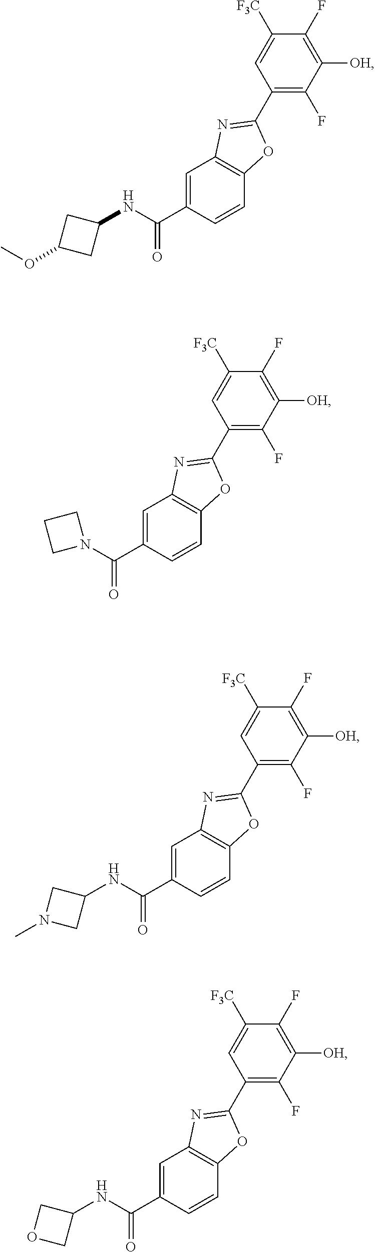 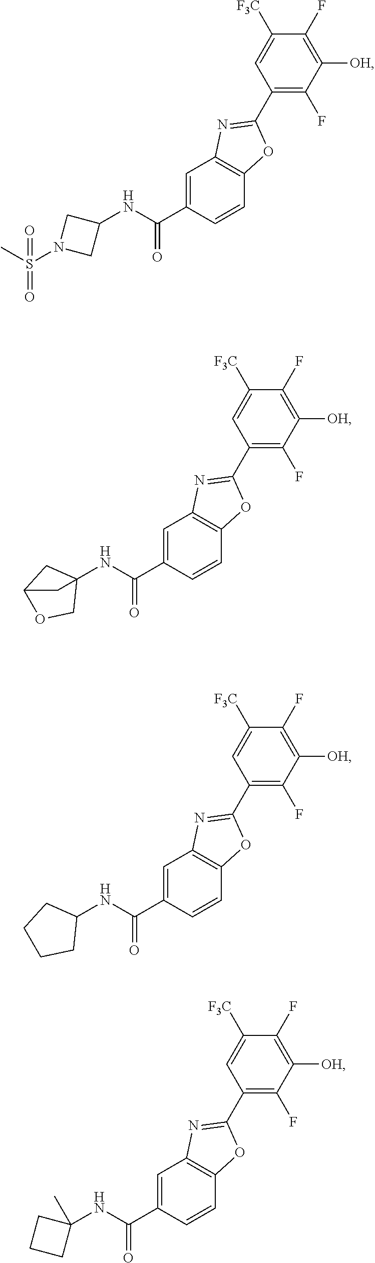 [SMILES: CS(=O)(=O)N1CC(NC(=O)c2ccc3oc(-c4cc(C(F)(F)F)c(F)c(O)c4F)nc3c2)C1 ; O=C(NC12COC(C1)C2)c1ccc2oc(-c3cc(C(F)(F)F)c(F)c(O)c3F)nc2c1 ; O=C(NC1CCCC1)c1ccc2oc(-c3cc(C(F)(F)F)c(F)c(O)c3F)nc2c1 ; CC1(NC(=O)c2ccc3oc(-c4cc(C(F)(F)F)c(F)c(O)c4F)nc3c2)CCC1] 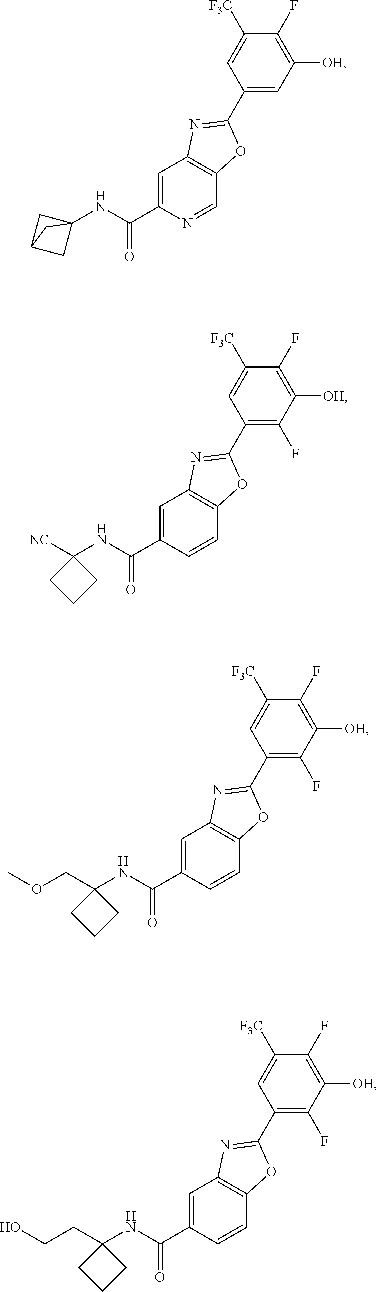 [SMILES: O=C(NC12CC(C1)C2)c1cc2nc(-c3cc(O)c(F)c(C(F)(F)F)c3)oc2cn1 ; N#CC1(NC(=O)c2ccc3oc(-c4cc(C(F)(F)F)c(F)c(O)c4F)nc3c2)CCC1 ; COCC1(NC(=O)c2ccc3oc(-c4cc(C(F)(F)F)c(F)c(O)c4F)nc3c2)CCC1 ; O=C(NC1(CCO)CCC1)c1ccc2oc(-c3cc(C(F)(F)F)c(F)c(O)c3F)nc2c1] 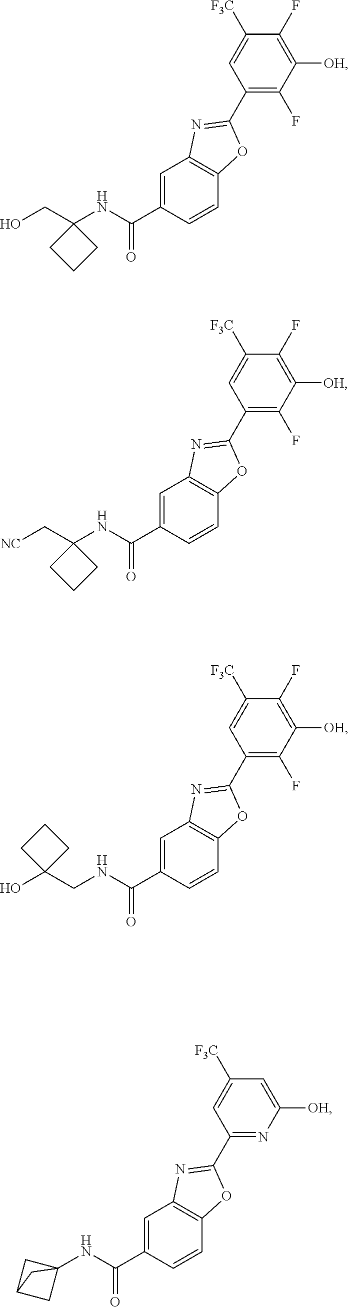 [SMILES: O=C(NC1(CO)CCC1)c1ccc2oc(-c3cc(C(F)(F)F)c(F)c(O)c3F)nc2c1 ; N#CCC1(NC(=O)c2ccc3oc(-c4cc(C(F)(F)F)c(F)c(O)c4F)nc3c2)CCC1 ; O=C(NCC1(O)CCC1)c1ccc2oc(-c3cc(C(F)(F)F)c(F)c(O)c3F)nc2c1 ; O=C(NC12CC(C1)C2)c1ccc2oc(-c3cc(C(F)(F)F)cc(O)n3)nc2c1] 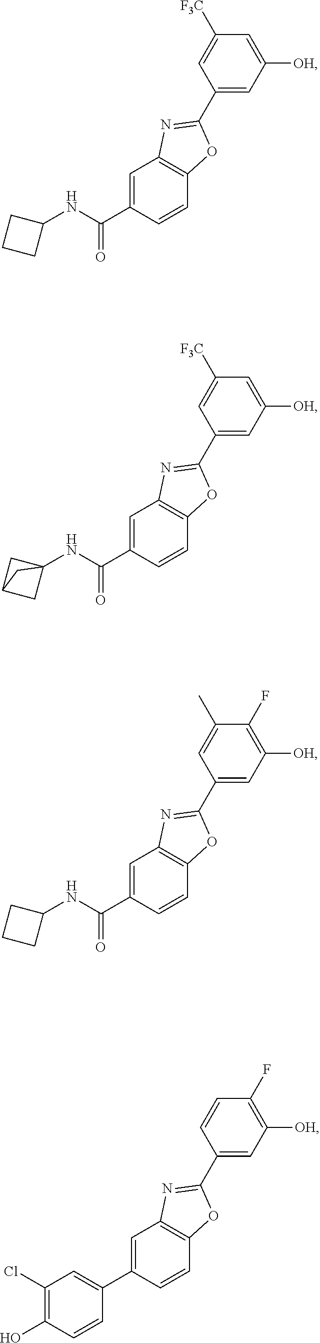 [SMILES: O=C(NC1CCC1)c1ccc2oc(-c3cc(O)cc(C(F)(F)F)c3)nc2c1 ; O=C(NC12CC(C1)C2)c1ccc2oc(-c3cc(O)cc(C(F)(F)F)c3)nc2c1 ; Cc1cc(-c2nc3cc(C(=O)NC4CCC4)ccc3o2)cc(O)c1F ; Oc1cc(-c2nc3cc(-c4ccc(O)c(Cl)c4)ccc3o2)ccc1F] 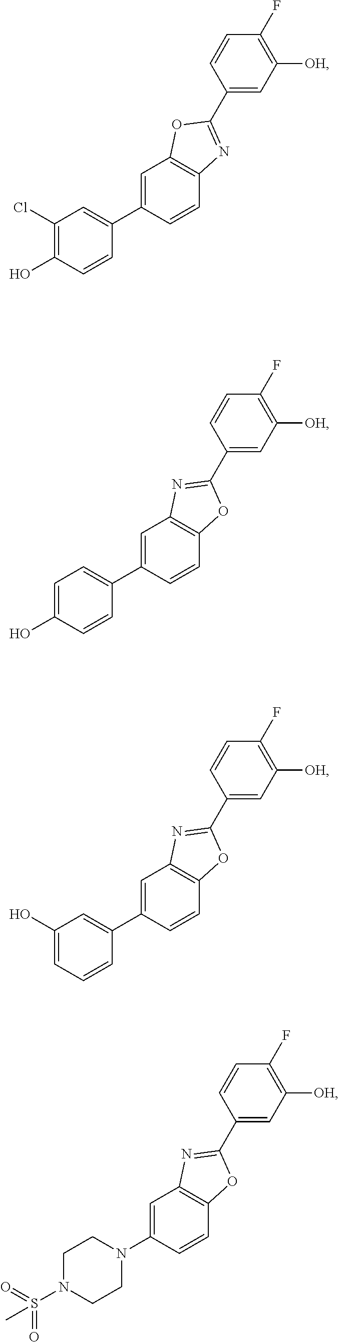 [SMILES: Oc1cc(-c2nc3ccc(-c4ccc(O)c(Cl)c4)cc3o2)ccc1F ; Oc1ccc(-c2ccc3oc(-c4ccc(F)c(O)c4)nc3c2)cc1 ; Oc1cccc(-c2ccc3oc(-c4ccc(F)c(O)c4)nc3c2)c1 ; CS(=O)(=O)N1CCN(c2ccc3oc(-c4ccc(F)c(O)c4)nc3c2)CC1] 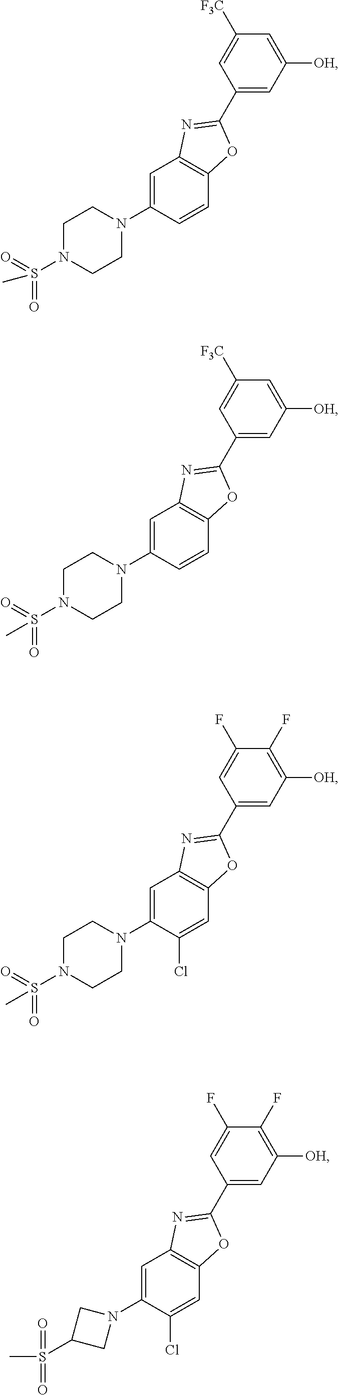 [SMILES: CS(=O)(=O)N1CCN(c2ccc3oc(-c4cc(O)cc(C(F)(F)F)c4)nc3c2)CC1 ; CS(=O)(=O)N1CCN(c2ccc3oc(-c4cc(O)cc(C(F)(F)F)c4)nc3c2)CC1 ; CS(=O)(=O)N1CCN(c2cc3nc(-c4cc(O)c(F)c(F)c4)oc3cc2Cl)CC1 ; CS(=O)(=O)C1CN(c2cc3nc(-c4cc(O)c(F)c(F)c4)oc3cc2Cl)C1] 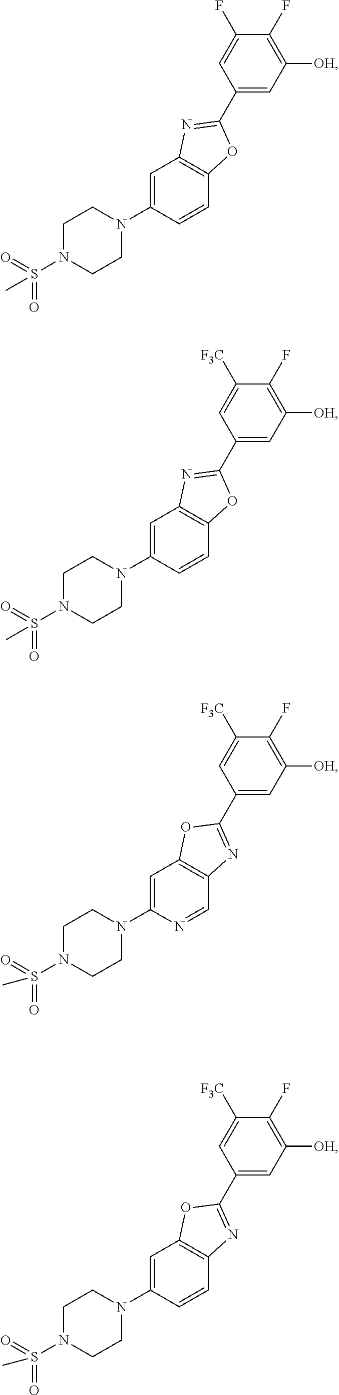 [SMILES: CS(=O)(=O)N1CCN(c2ccc3oc(-c4cc(O)c(F)c(F)c4)nc3c2)CC1 ; CS(=O)(=O)N1CCN(c2ccc3oc(-c4cc(O)c(F)c(C(F)(F)F)c4)nc3c2)CC1 ; CS(=O)(=O)N1CCN(c2cc3oc(-c4cc(O)c(F)c(C(F)(F)F)c4)nc3cn2)CC1 ; CS(=O)(=O)N1CCN(c2ccc3nc(-c4cc(O)c(F)c(C(F)(F)F)c4)oc3c2)CC1] 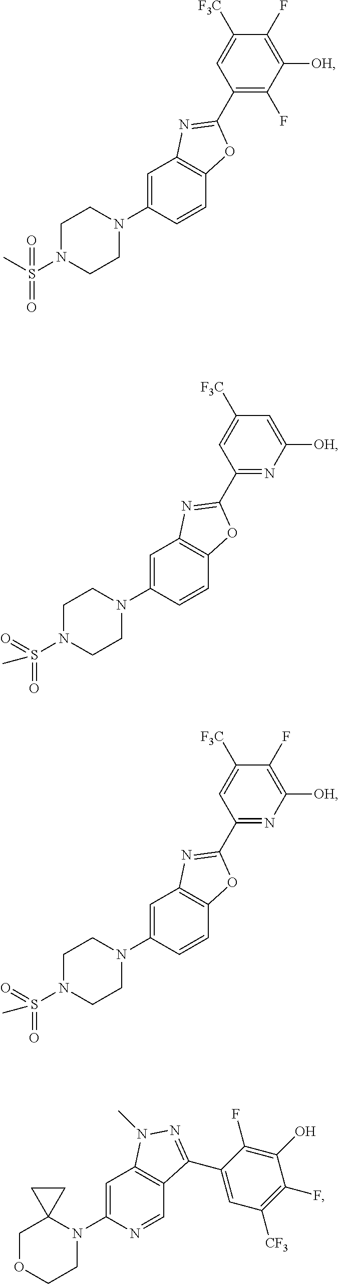 [SMILES: CS(=O)(=O)N1CCN(c2ccc3oc(-c4cc(C(F)(F)F)c(F)c(O)c4F)nc3c2)CC1 ; CS(=O)(=O)N1CCN(c2ccc3oc(-c4cc(C(F)(F)F)cc(O)n4)nc3c2)CC1 ; CS(=O)(=O)N1CCN(c2ccc3oc(-c4cc(C(F)(F)F)c(F)c(O)n4)nc3c2)CC1 ; Cn1nc(-c2cc(C(F)(F)F)c(F)c(O)c2F)c2cnc(N3CCOCC34CC4)cc21] 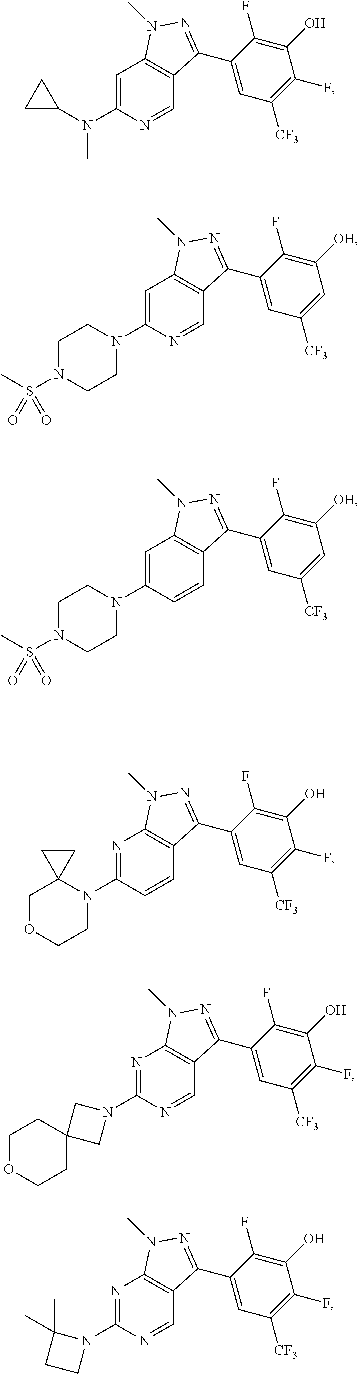 [SMILES: CN(c1cc2c(cn1)c(-c1cc(C(F)(F)F)c(F)c(O)c1F)nn2C)C1CC1 ; Cn1nc(-c2cc(C(F)(F)F)cc(O)c2F)c2cnc(N3CCN(S(C)(=O)=O)CC3)cc21 ; Cn1nc(-c2cc(C(F)(F)F)cc(O)c2F)c2ccc(N3CCN(S(C)(=O)=O)CC3)cc21 ; Cn1nc(-c2cc(C(F)(F)F)c(F)c(O)c2F)c2ccc(N3CCOCC34CC4)nc21 ; Cn1nc(-c2cc(C(F)(F)F)c(F)c(O)c2F)c2cnc(N3CC4(CCOCC4)C3)nc21 ; Cn1nc(-c2cc(C(F)(F)F)c(F)c(O)c2F)c2cnc(N3CCC3(C)C)nc21] 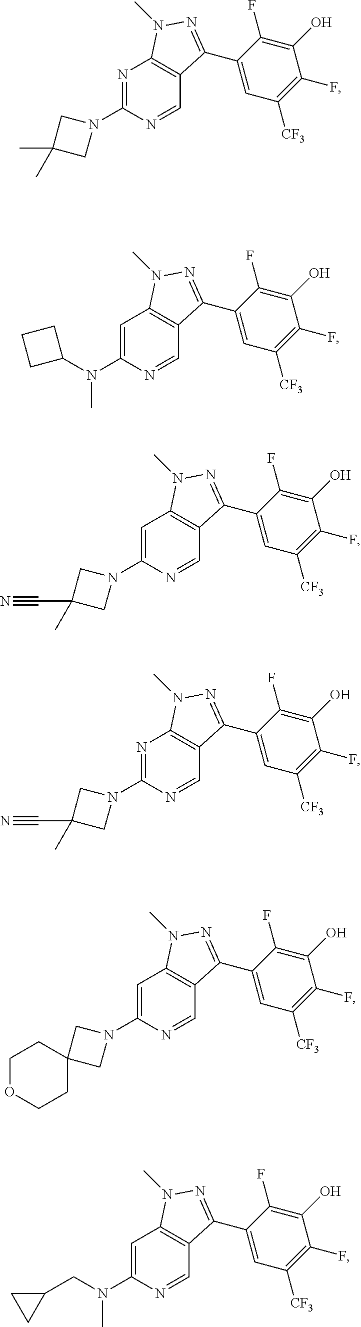 [SMILES: Cn1nc(-c2cc(C(F)(F)F)c(F)c(O)c2F)c2cnc(N3CC(C)(C)C3)nc21 ; CN(c1cc2c(cn1)c(-c1cc(C(F)(F)F)c(F)c(O)c1F)nn2C)C1CCC1 ; Cn1nc(-c2cc(C(F)(F)F)c(F)c(O)c2F)c2cnc(N3CC(C)(C#N)C3)cc21 ; Cn1nc(-c2cc(C(F)(F)F)c(F)c(O)c2F)c2cnc(N3CC(C)(C#N)C3)nc21 ; Cn1nc(-c2cc(C(F)(F)F)c(F)c(O)c2F)c2cnc(N3CC4(CCOCC4)C3)cc21 ; CN(CC1CC1)c1cc2c(cn1)c(-c1cc(C(F)(F)F)c(F)c(O)c1F)nn2C] 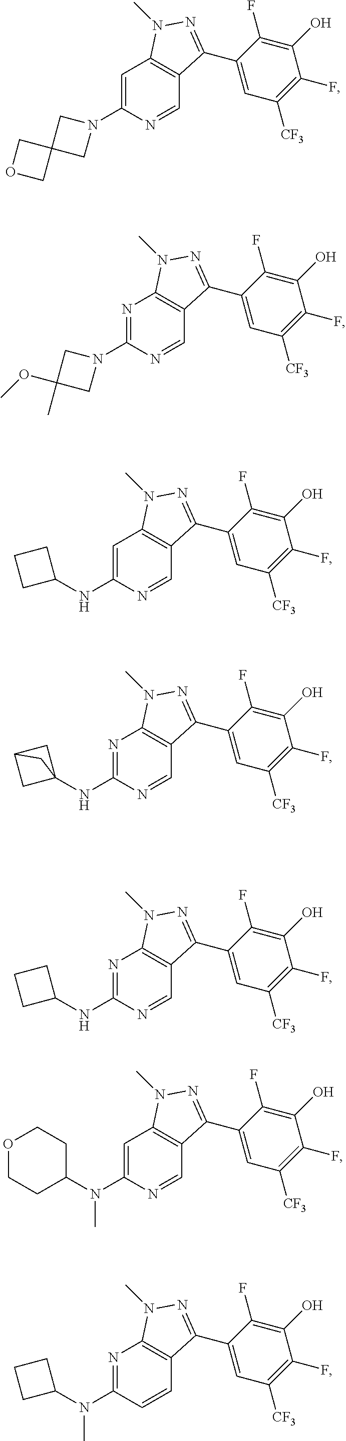 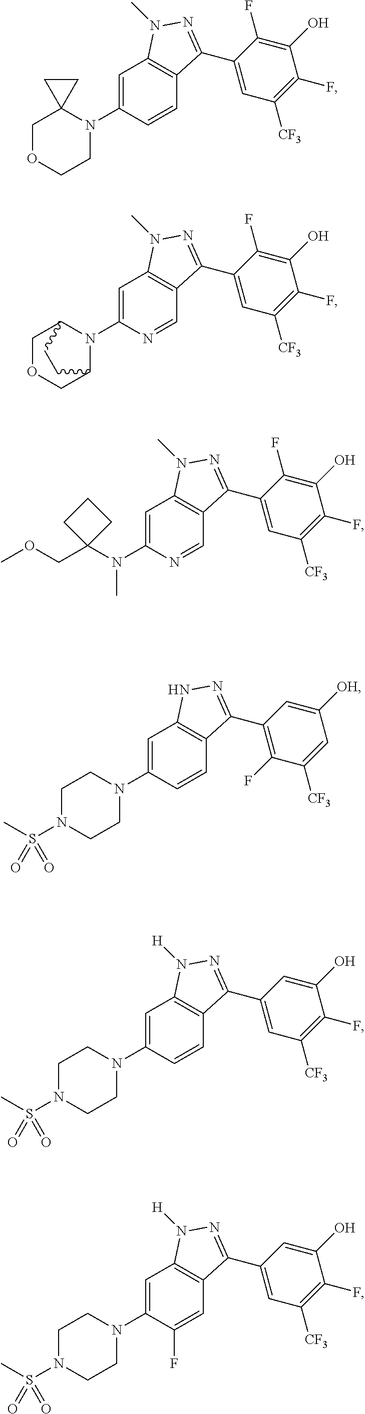 [SMILES: Cn1nc(-c2cc(C(F)(F)F)c(F)c(O)c2F)c2ccc(N3CCOCC34CC4)cc21 ; Cn1nc(-c2cc(C(F)(F)F)c(F)c(O)c2F)c2cnc(N3C4CCC3COC4)cc21 ; COCC1(N(C)c2cc3c(cn2)c(-c2cc(C(F)(F)F)c(F)c(O)c2F)nn3C)CCC1 ; CS(=O)(=O)N1CCN(c2ccc3c(-c4cc(O)cc(C(F)(F)F)c4F)n[nH]c3c2)CC1 ; CS(=O)(=O)N1CCN(c2ccc3c(-c4cc(O)c(F)c(C(F)(F)F)c4)n[nH]c3c2)CC1 ; CS(=O)(=O)N1CCN(c2cc3[nH]nc(-c4cc(O)c(F)c(C(F)(F)F)c4)c3cc2F)CC1] 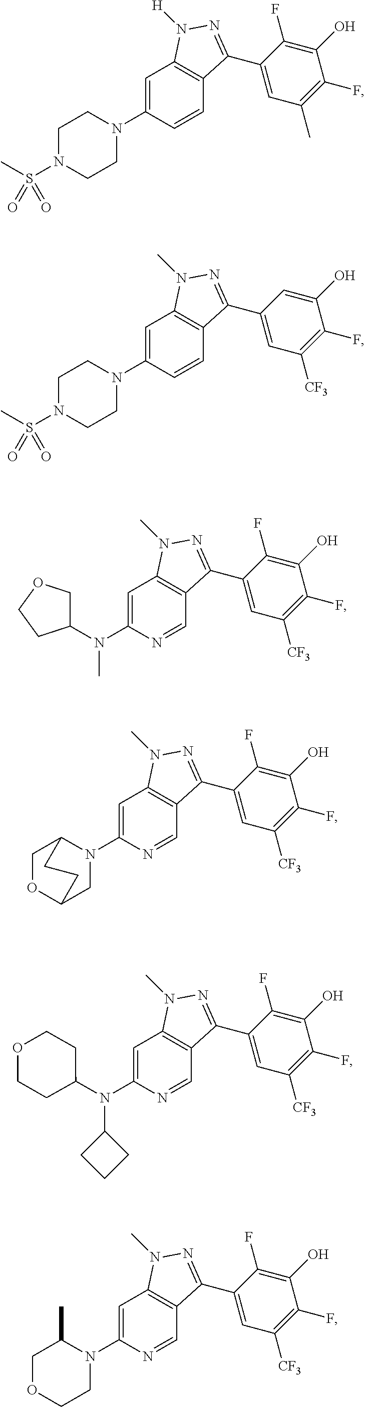 [SMILES: Cc1cc(-c2n[nH]c3cc(N4CCN(S(C)(=O)=O)CC4)ccc23)c(F)c(O)c1F ; Cn1nc(-c2cc(O)c(F)c(C(F)(F)F)c2)c2ccc(N3CCN(S(C)(=O)=O)CC3)cc21 ; CN(c1cc2c(cn1)c(-c1cc(C(F)(F)F)c(F)c(O)c1F)nn2C)C1CCOC1 ; Cn1nc(-c2cc(C(F)(F)F)c(F)c(O)c2F)c2cnc(N3CC4CCC3CO4)cc21 ; Cn1nc(-c2cc(C(F)(F)F)c(F)c(O)c2F)c2cnc(N(C3CCC3)C3CCOCC3)cc21 ; C[C@@H]1COCCN1c1cc2c(cn1)c(-c1cc(C(F)(F)F)c(F)c(O)c1F)nn2C] 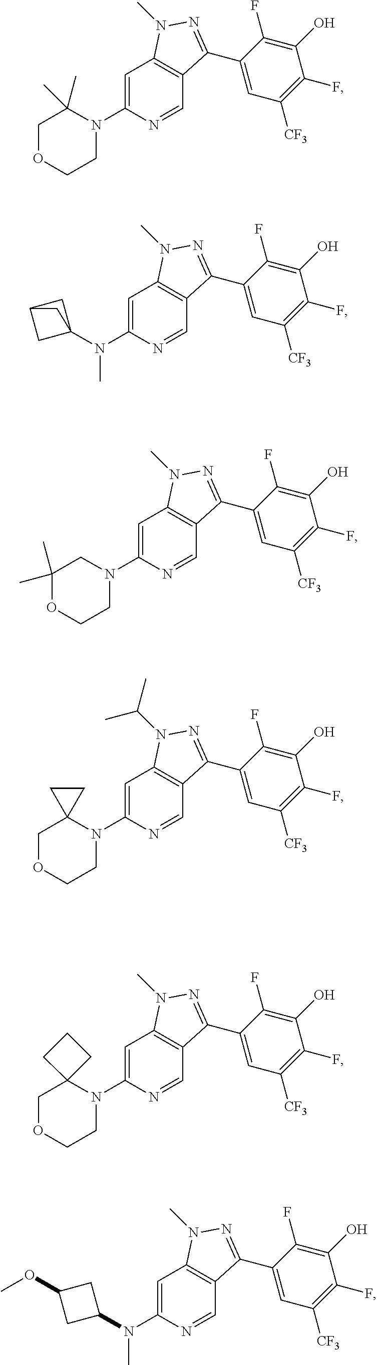 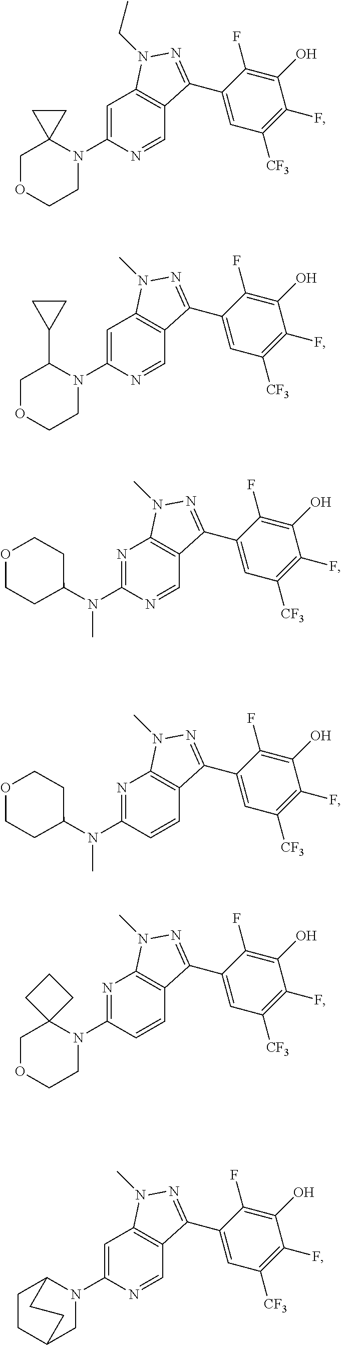 [SMILES: CCn1nc(-c2cc(C(F)(F)F)c(F)c(O)c2F)c2cnc(N3CCOCC34CC4)cc21 ; Cn1nc(-c2cc(C(F)(F)F)c(F)c(O)c2F)c2cnc(N3CCOCC3C3CC3)cc21 ; CN(c1ncc2c(-c3cc(C(F)(F)F)c(F)c(O)c3F)nn(C)c2n1)C1CCOCC1 ; CN(c1ccc2c(-c3cc(C(F)(F)F)c(F)c(O)c3F)nn(C)c2n1)C1CCOCC1 ; Cn1nc(-c2cc(C(F)(F)F)c(F)c(O)c2F)c2ccc(N3CCOCC34CCC4)nc21 ; Cn1nc(-c2cc(C(F)(F)F)c(F)c(O)c2F)c2cnc(N3CC4CCC3CC4)cc21] 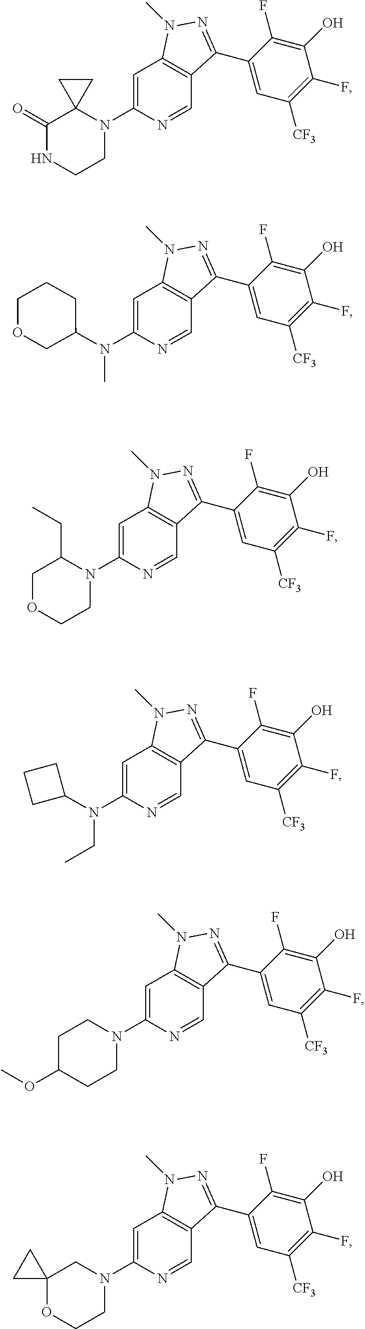 [SMILES: Cn1nc(-c2cc(C(F)(F)F)c(F)c(O)c2F)c2cnc(N3CCNC(=O)C34CC4)cc21 ; CN(c1cc2c(cn1)c(-c1cc(C(F)(F)F)c(F)c(O)c1F)nn2C)C1CCCOC1 ; CCC1COCCN1c1cc2c(cn1)c(-c1cc(C(F)(F)F)c(F)c(O)c1F)nn2C ; CCN(c1cc2c(cn1)c(-c1cc(C(F)(F)F)c(F)c(O)c1F)nn2C)C1CCC1 ; COC1CCN(c2cc3c(cn2)c(-c2cc(C(F)(F)F)c(F)c(O)c2F)nn3C)CC1 ; Cn1nc(-c2cc(C(F)(F)F)c(F)c(O)c2F)c2cnc(N3CCOC4(CC4)C3)cc21] 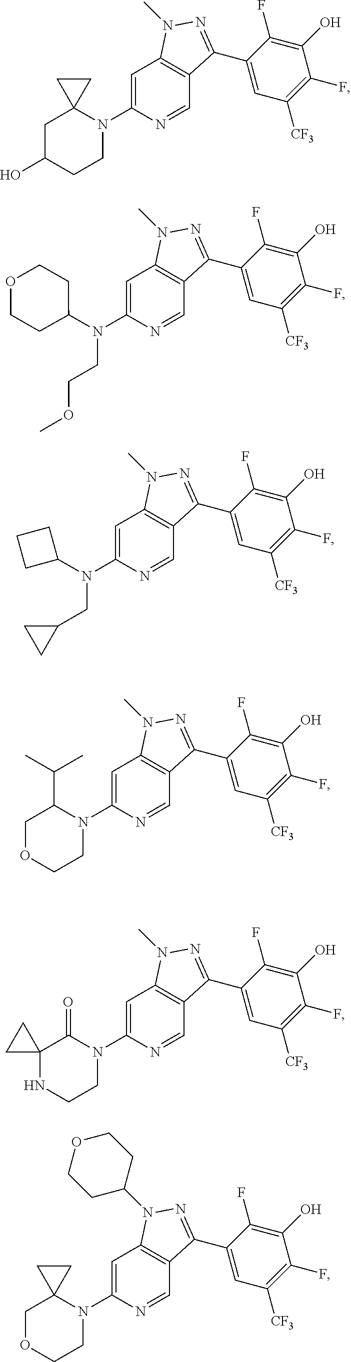 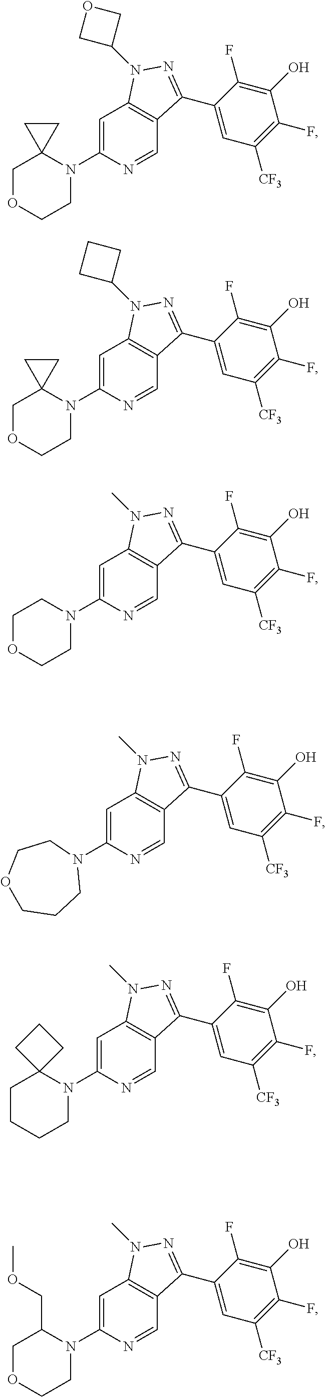 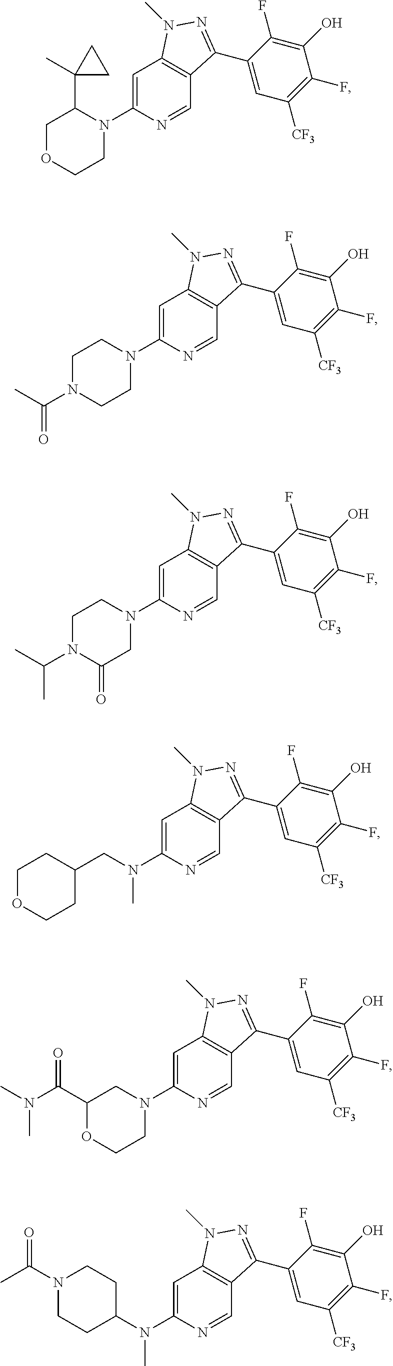 [SMILES: Cn1nc(-c2cc(C(F)(F)F)c(F)c(O)c2F)c2cnc(N3CCOCC3C3(C)CC3)cc21 ; CC(=O)N1CCN(c2cc3c(cn2)c(-c2cc(C(F)(F)F)c(F)c(O)c2F)nn3C)CC1 ; CC(C)N1CCN(c2cc3c(cn2)c(-c2cc(C(F)(F)F)c(F)c(O)c2F)nn3C)CC1=O ; CN(CC1CCOCC1)c1cc2c(cn1)c(-c1cc(C(F)(F)F)c(F)c(O)c1F)nn2C ; CN(C)C(=O)C1CN(c2cc3c(cn2)c(-c2cc(C(F)(F)F)c(F)c(O)c2F)nn3C)CCO1 ; CC(=O)N1CCC(N(C)c2cc3c(cn2)c(-c2cc(C(F)(F)F)c(F)c(O)c2F)nn3C)CC1] 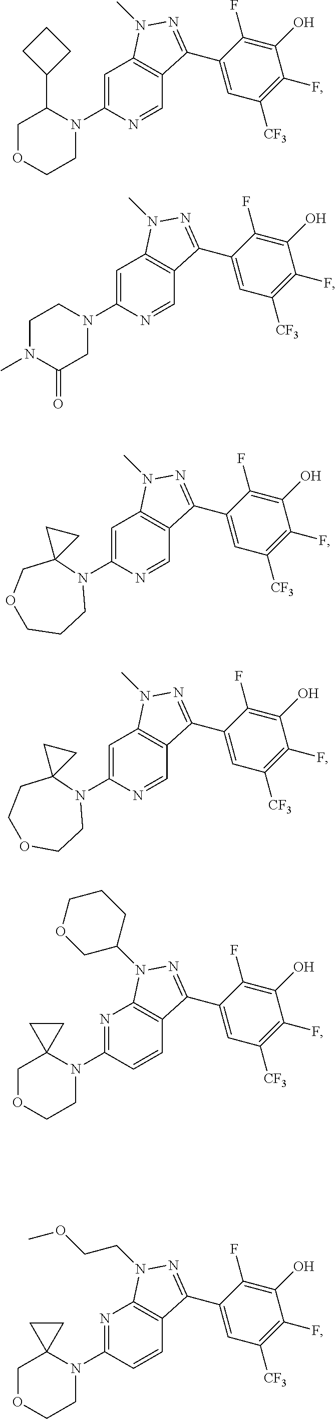 [SMILES: Cn1nc(-c2cc(C(F)(F)F)c(F)c(O)c2F)c2cnc(N3CCOCC3C3CCC3)cc21 ; CN1CCN(c2cc3c(cn2)c(-c2cc(C(F)(F)F)c(F)c(O)c2F)nn3C)CC1=O ; Cn1nc(-c2cc(C(F)(F)F)c(F)c(O)c2F)c2cnc(N3CCCOCC34CC4)cc21 ; Cn1nc(-c2cc(C(F)(F)F)c(F)c(O)c2F)c2cnc(N3CCOCCC34CC4)cc21 ; Oc1c(F)c(-c2nn(C3CCCOC3)c3nc(N4CCOCC45CC5)ccc23)cc(C(F)(F)F)c1F ; COCCn1nc(-c2cc(C(F)(F)F)c(F)c(O)c2F)c2ccc(N3CCOCC34CC4)nc21] 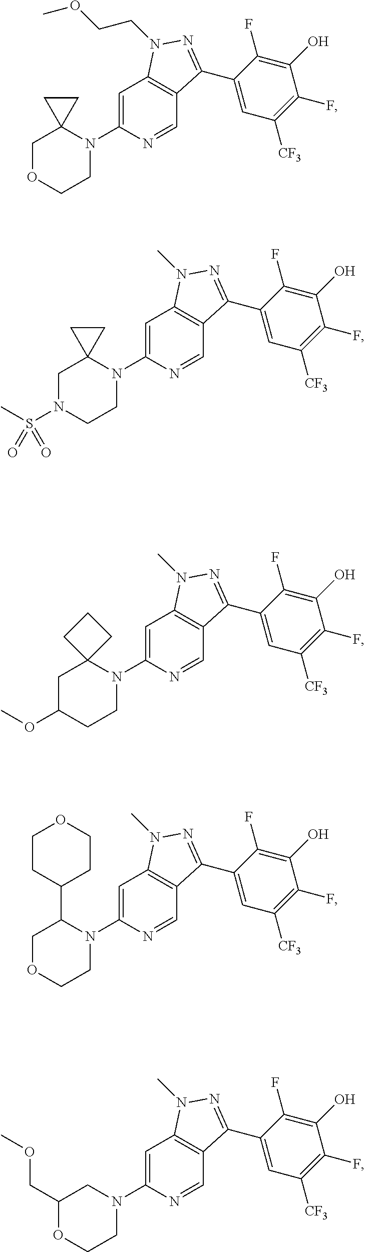 [SMILES: COCCn1nc(-c2cc(C(F)(F)F)c(F)c(O)c2F)c2cnc(N3CCOCC34CC4)cc21 ; Cn1nc(-c2cc(C(F)(F)F)c(F)c(O)c2F)c2cnc(N3CCN(S(C)(=O)=O)CC34CC4)cc21 ; COC1CCN(c2cc3c(cn2)c(-c2cc(C(F)(F)F)c(F)c(O)c2F)nn3C)C2(CCC2)C1 ; Cn1nc(-c2cc(C(F)(F)F)c(F)c(O)c2F)c2cnc(N3CCOCC3C3CCOCC3)cc21 ; COCC1CN(c2cc3c(cn2)c(-c2cc(C(F)(F)F)c(F)c(O)c2F)nn3C)CCO1] 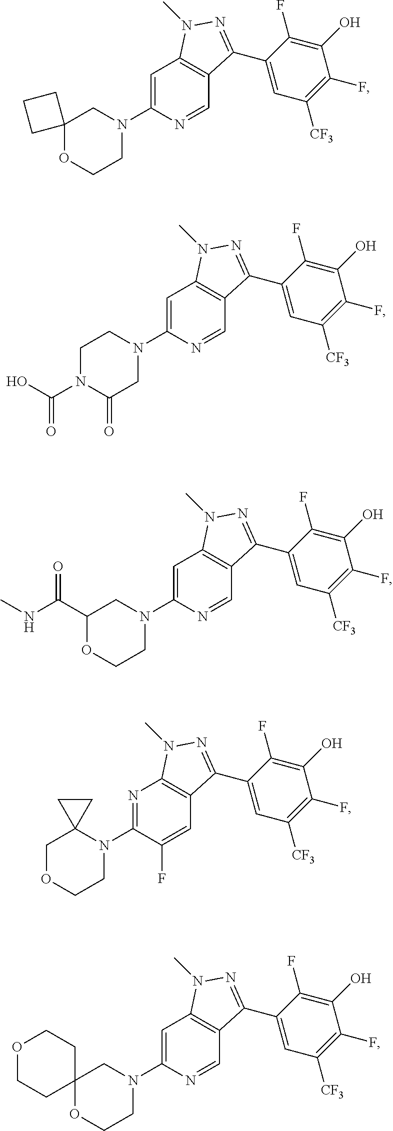 [SMILES: Cn1nc(-c2cc(C(F)(F)F)c(F)c(O)c2F)c2cnc(N3CCOC4(CCC4)C3)cc21 ; Cn1nc(-c2cc(C(F)(F)F)c(F)c(O)c2F)c2cnc(N3CCN(C(=O)O)C(=O)C3)cc21 ; CNC(=O)C1CN(c2cc3c(cn2)c(-c2cc(C(F)(F)F)c(F)c(O)c2F)nn3C)CCO1 ; Cn1nc(-c2cc(C(F)(F)F)c(F)c(O)c2F)c2cc(F)c(N3CCOCC34CC4)nc21 ; Cn1nc(-c2cc(C(F)(F)F)c(F)c(O)c2F)c2cnc(N3CCOC4(CCOCC4)C3)cc21] 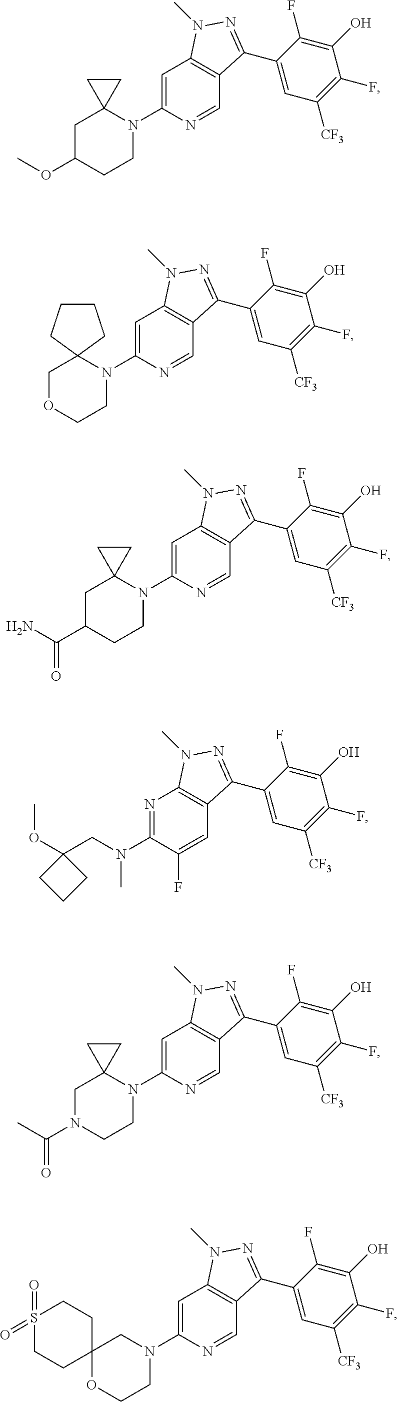 [SMILES: COC1CCN(c2cc3c(cn2)c(-c2cc(C(F)(F)F)c(F)c(O)c2F)nn3C)C2(CC2)C1 ; Cn1nc(-c2cc(C(F)(F)F)c(F)c(O)c2F)c2cnc(N3CCOCC34CCCC4)cc21 ; Cn1nc(-c2cc(C(F)(F)F)c(F)c(O)c2F)c2cnc(N3CCC(C(N)=O)CC34CC4)cc21 ; COC1(CN(C)c2nc3c(cc2F)c(-c2cc(C(F)(F)F)c(F)c(O)c2F)nn3C)CCC1 ; CC(=O)N1CCN(c2cc3c(cn2)c(-c2cc(C(F)(F)F)c(F)c(O)c2F)nn3C)C2(CC2)C1 ; Cn1nc(-c2cc(C(F)(F)F)c(F)c(O)c2F)c2cnc(N3CCOC4(CCS(=O)(=O)CC4)C3)cc21] 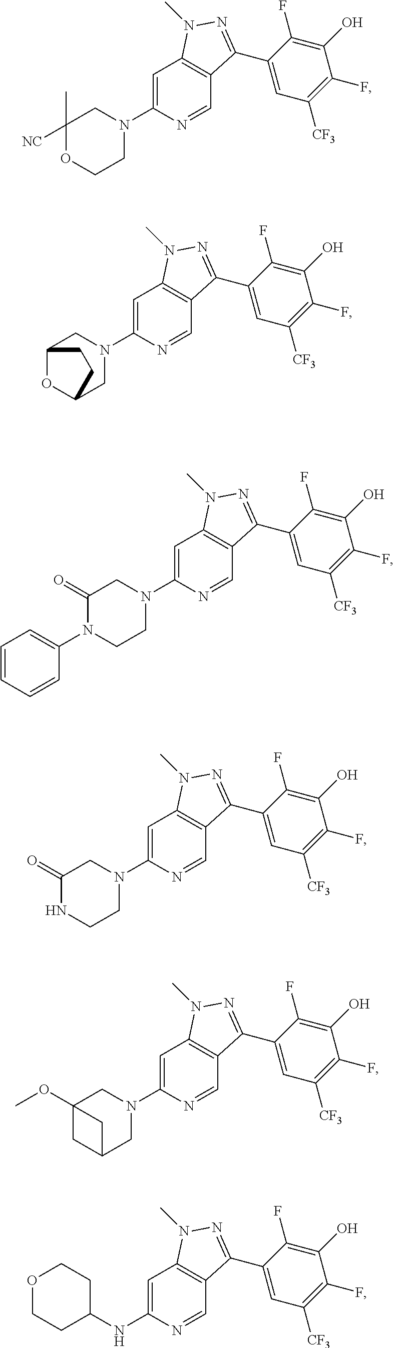 [SMILES: Cn1nc(-c2cc(C(F)(F)F)c(F)c(O)c2F)c2cnc(N3CCOC(C)(C#N)C3)cc21 ; Cn1nc(-c2cc(C(F)(F)F)c(F)c(O)c2F)c2cnc(N3C[C@H]4CC[C@@H](C3)O4)cc21 ; Cn1nc(-c2cc(C(F)(F)F)c(F)c(O)c2F)c2cnc(N3CCN(c4ccccc4)C(=O)C3)cc21 ; Cn1nc(-c2cc(C(F)(F)F)c(F)c(O)c2F)c2cnc(N3CCNC(=O)C3)cc21 ; COC12CC(CN(c3cc4c(cn3)c(-c3cc(C(F)(F)F)c(F)c(O)c3F)nn4C)C1)C2 ; Cn1nc(-c2cc(C(F)(F)F)c(F)c(O)c2F)c2cnc(NC3CCOCC3)cc21] 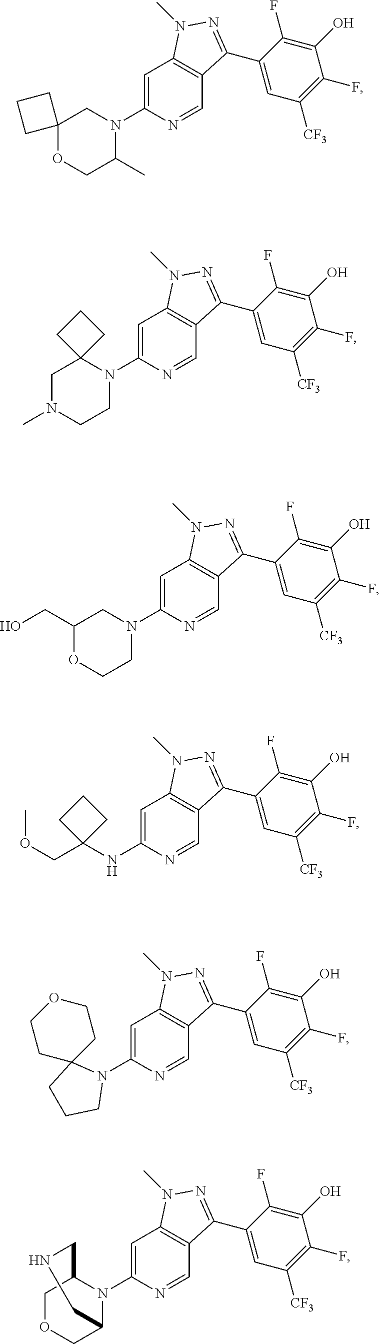 [SMILES: CC1COC2(CCC2)CN1c1cc2c(cn1)c(-c1cc(C(F)(F)F)c(F)c(O)c1F)nn2C ; CN1CCN(c2cc3c(cn2)c(-c2cc(C(F)(F)F)c(F)c(O)c2F)nn3C)C2(CCC2)C1 ; Cn1nc(-c2cc(C(F)(F)F)c(F)c(O)c2F)c2cnc(N3CCOC(CO)C3)cc21 ; COCC1(Nc2cc3c(cn2)c(-c2cc(C(F)(F)F)c(F)c(O)c2F)nn3C)CCC1 ; Cn1nc(-c2cc(C(F)(F)F)c(F)c(O)c2F)c2cnc(N3CCCC34CCOCC4)cc21 ; Cn1nc(-c2cc(C(F)(F)F)c(F)c(O)c2F)c2cnc(N3[C@@H]4CNC[C@H]3COC4)cc21] 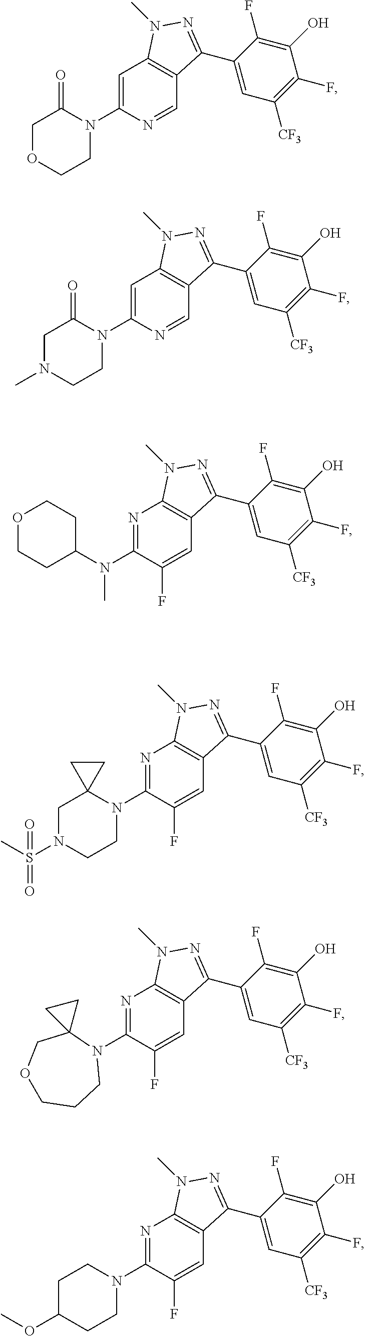 [SMILES: Cn1nc(-c2cc(C(F)(F)F)c(F)c(O)c2F)c2cnc(N3CCOCC3=O)cc21 ; CN1CCN(c2cc3c(cn2)c(-c2cc(C(F)(F)F)c(F)c(O)c2F)nn3C)C(=O)C1 ; CN(c1nc2c(cc1F)c(-c1cc(C(F)(F)F)c(F)c(O)c1F)nn2C)C1CCOCC1 ; Cn1nc(-c2cc(C(F)(F)F)c(F)c(O)c2F)c2cc(F)c(N3CCN(S(C)(=O)=O)CC34CC4)nc21 ; Cn1nc(-c2cc(C(F)(F)F)c(F)c(O)c2F)c2cc(F)c(N3CCCOCC34CC4)nc21 ; COC1CCN(c2nc3c(cc2F)c(-c2cc(C(F)(F)F)c(F)c(O)c2F)nn3C)CC1] 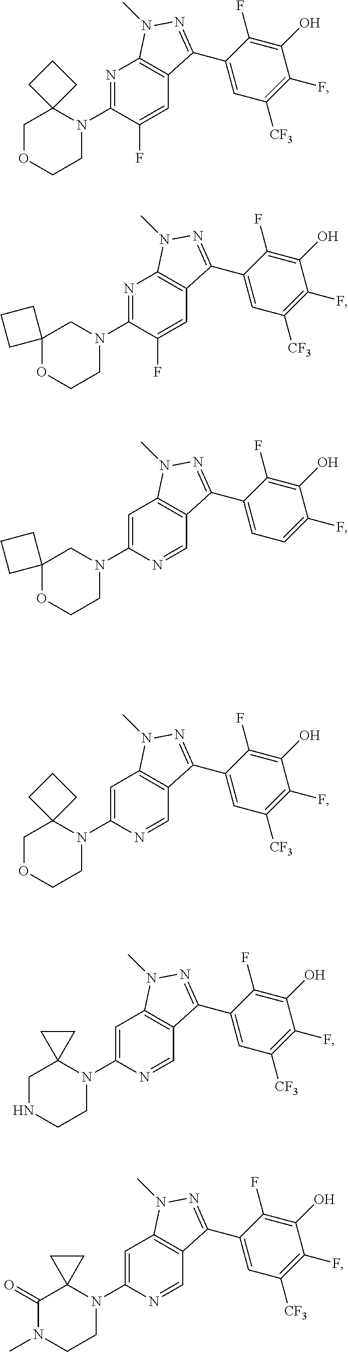 [SMILES: Cn1nc(-c2cc(C(F)(F)F)c(F)c(O)c2F)c2cc(F)c(N3CCOCC34CCC4)nc21 ; Cn1nc(-c2cc(C(F)(F)F)c(F)c(O)c2F)c2cc(F)c(N3CCOC4(CCC4)C3)nc21 ; Cn1nc(-c2ccc(F)c(O)c2F)c2cnc(N3CCOC4(CCC4)C3)cc21 ; Cn1nc(-c2cc(C(F)(F)F)c(F)c(O)c2F)c2cnc(N3CCOCC34CCC4)cc21 ; Cn1nc(-c2cc(C(F)(F)F)c(F)c(O)c2F)c2cnc(N3CCNCC34CC4)cc21 ; CN1CCN(c2cc3c(cn2)c(-c2cc(C(F)(F)F)c(F)c(O)c2F)nn3C)C2(CC2)C1=O] 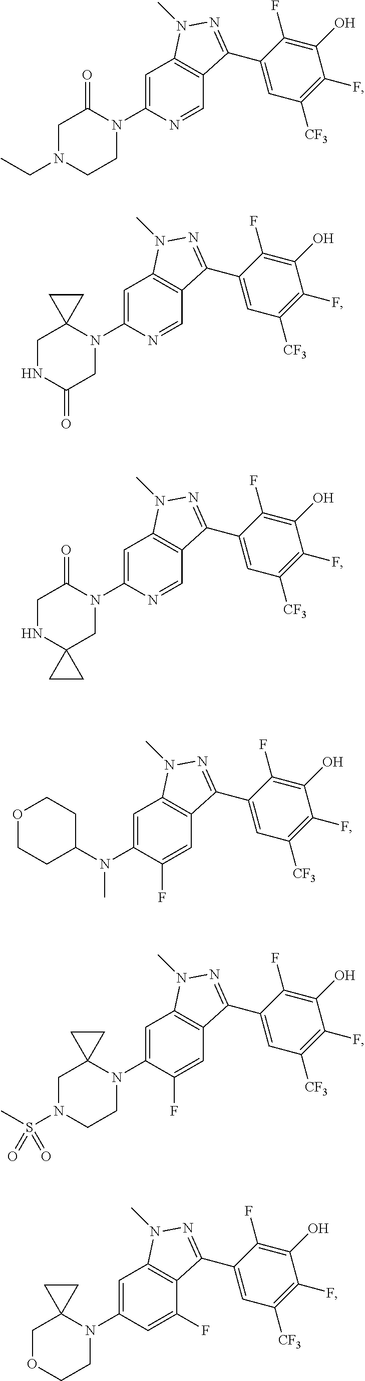 [SMILES: CCN1CCN(c2cc3c(cn2)c(-c2cc(C(F)(F)F)c(F)c(O)c2F)nn3C)C(=O)C1 ; Cn1nc(-c2cc(C(F)(F)F)c(F)c(O)c2F)c2cnc(N3CC(=O)NCC34CC4)cc21 ; Cn1nc(-c2cc(C(F)(F)F)c(F)c(O)c2F)c2cnc(N3CC4(CC4)NCC3=O)cc21 ; CN(c1cc2c(cc1F)c(-c1cc(C(F)(F)F)c(F)c(O)c1F)nn2C)C1CCOCC1 ; Cn1nc(-c2cc(C(F)(F)F)c(F)c(O)c2F)c2cc(F)c(N3CCN(S(C)(=O)=O)CC34CC4)cc21 ; Cn1nc(-c2cc(C(F)(F)F)c(F)c(O)c2F)c2c(F)cc(N3CCOCC34CC4)cc21] 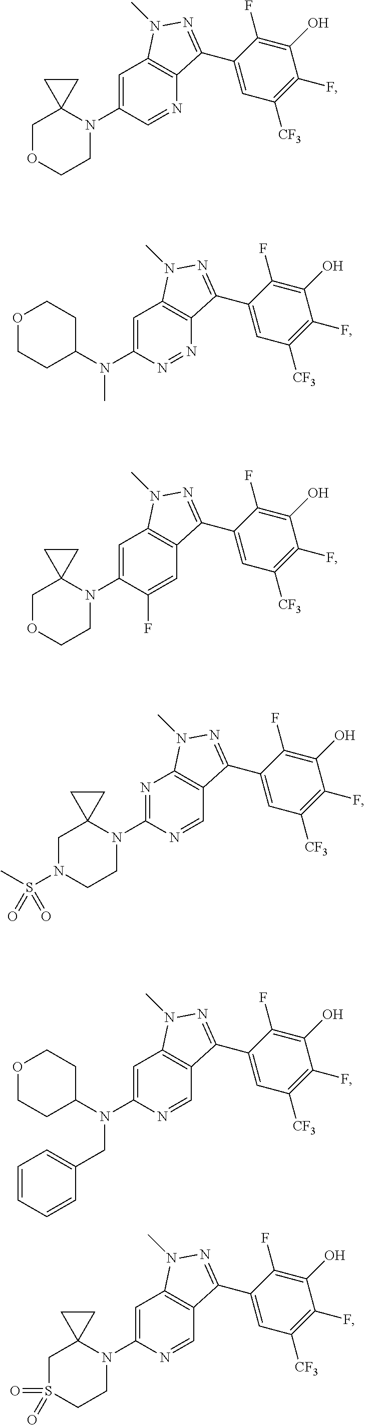 [SMILES: Cn1nc(-c2cc(C(F)(F)F)c(F)c(O)c2F)c2ncc(N3CCOCC34CC4)cc21 ; CN(c1cc2c(nn1)c(-c1cc(C(F)(F)F)c(F)c(O)c1F)nn2C)C1CCOCC1 ; Cn1nc(-c2cc(C(F)(F)F)c(F)c(O)c2F)c2cc(F)c(N3CCOCC34CC4)cc21 ; Cn1nc(-c2cc(C(F)(F)F)c(F)c(O)c2F)c2cnc(N3CCN(S(C)(=O)=O)CC34CC4)nc21 ; Cn1nc(-c2cc(C(F)(F)F)c(F)c(O)c2F)c2cnc(N(Cc3ccccc3)C3CCOCC3)cc21 ; Cn1nc(-c2cc(C(F)(F)F)c(F)c(O)c2F)c2cnc(N3CCS(=O)(=O)CC34CC4)cc21] 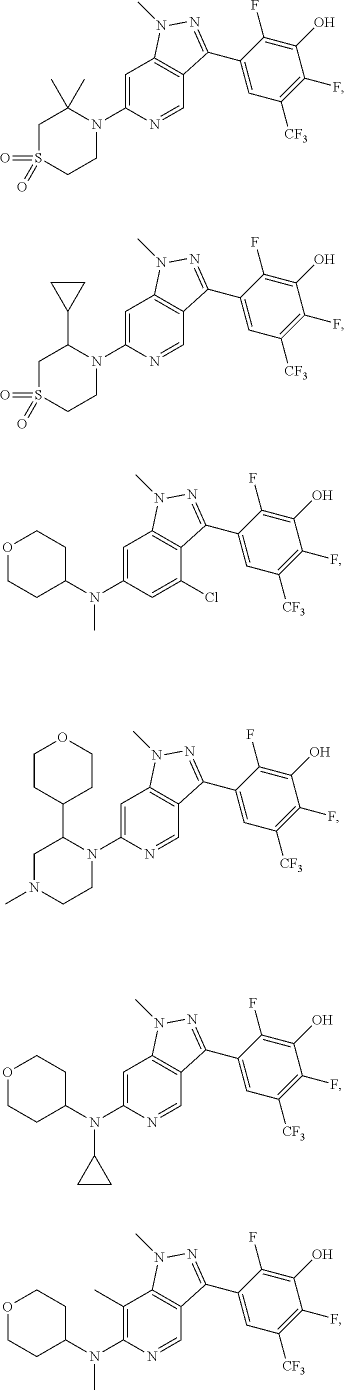 [SMILES: Cn1nc(-c2cc(C(F)(F)F)c(F)c(O)c2F)c2cnc(N3CCS(=O)(=O)CC3(C)C)cc21 ; Cn1nc(-c2cc(C(F)(F)F)c(F)c(O)c2F)c2cnc(N3CCS(=O)(=O)CC3C3CC3)cc21 ; CN(c1cc(Cl)c2c(-c3cc(C(F)(F)F)c(F)c(O)c3F)nn(C)c2c1)C1CCOCC1 ; CN1CCN(c2cc3c(cn2)c(-c2cc(C(F)(F)F)c(F)c(O)c2F)nn3C)C(C2CCOCC2)C1 ; Cn1nc(-c2cc(C(F)(F)F)c(F)c(O)c2F)c2cnc(N(C3CCOCC3)C3CC3)cc21 ; Cc1c(N(C)C2CCOCC2)ncc2c(-c3cc(C(F)(F)F)c(F)c(O)c3F)nn(C)c12] 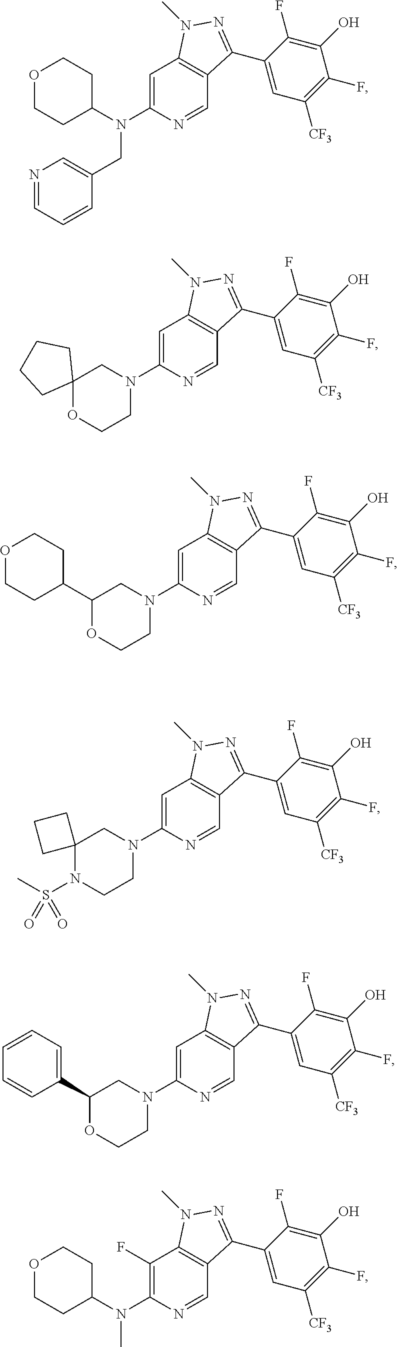 [SMILES: Cn1nc(-c2cc(C(F)(F)F)c(F)c(O)c2F)c2cnc(N(Cc3cccnc3)C3CCOCC3)cc21 ; Cn1nc(-c2cc(C(F)(F)F)c(F)c(O)c2F)c2cnc(N3CCOC4(CCCC4)C3)cc21 ; Cn1nc(-c2cc(C(F)(F)F)c(F)c(O)c2F)c2cnc(N3CCOC(C4CCOCC4)C3)cc21 ; Cn1nc(-c2cc(C(F)(F)F)c(F)c(O)c2F)c2cnc(N3CCN(S(C)(=O)=O)C4(CCC4)C3)cc21 ; Cn1nc(-c2cc(C(F)(F)F)c(F)c(O)c2F)c2cnc(N3CCO[C@@H](c4ccccc4)C3)cc21 ; CN(c1ncc2c(-c3cc(C(F)(F)F)c(F)c(O)c3F)nn(C)c2c1F)C1CCOCC1] 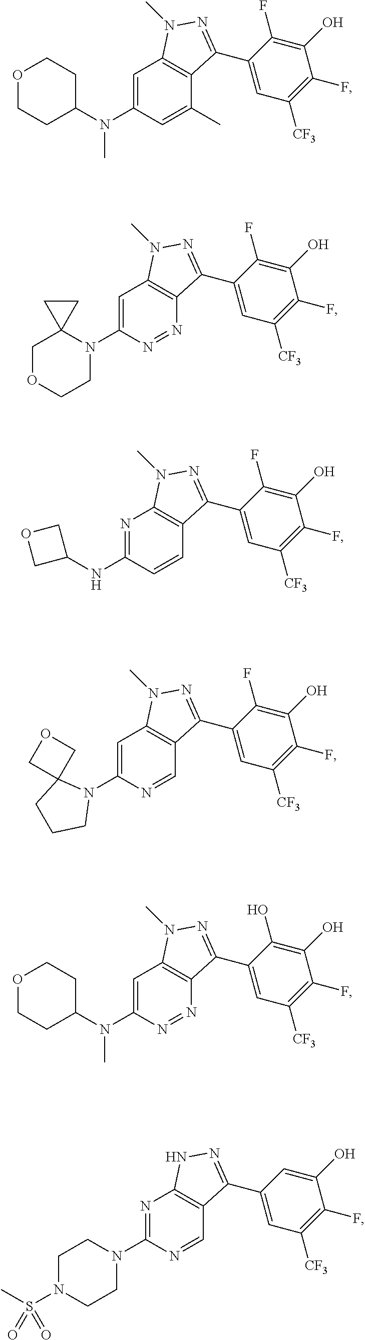 [SMILES: Cc1cc(N(C)C2CCOCC2)cc2c1c(-c1cc(C(F)(F)F)c(F)c(O)c1F)nn2C ; Cn1nc(-c2cc(C(F)(F)F)c(F)c(O)c2F)c2nnc(N3CCOCC34CC4)cc21 ; Cn1nc(-c2cc(C(F)(F)F)c(F)c(O)c2F)c2ccc(NC3COC3)nc21 ; Cn1nc(-c2cc(C(F)(F)F)c(F)c(O)c2F)c2cnc(N3CCCC34COC4)cc21 ; CN(c1cc2c(nn1)c(-c1cc(C(F)(F)F)c(F)c(O)c1O)nn2C)C1CCOCC1 ; CS(=O)(=O)N1CCN(c2ncc3c(-c4cc(O)c(F)c(C(F)(F)F)c4)n[nH]c3n2)CC1] 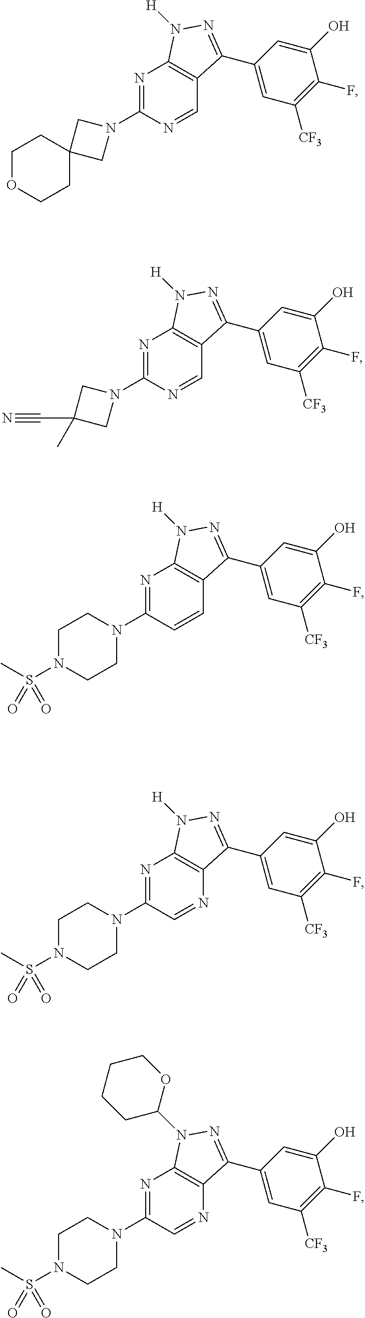 [SMILES: Oc1cc(-c2n[nH]c3nc(N4CC5(CCOCC5)C4)ncc23)cc(C(F)(F)F)c1F ; CC1(C#N)CN(c2ncc3c(-c4cc(O)c(F)c(C(F)(F)F)c4)n[nH]c3n2)C1 ; CS(=O)(=O)N1CCN(c2ccc3c(-c4cc(O)c(F)c(C(F)(F)F)c4)n[nH]c3n2)CC1 ; CS(=O)(=O)N1CCN(c2cnc3c(-c4cc(O)c(F)c(C(F)(F)F)c4)n[nH]c3n2)CC1 ; CS(=O)(=O)N1CCN(c2cnc3c(-c4cc(O)c(F)c(C(F)(F)F)c4)nn(C4CCCCO4)c3n2)CC1] 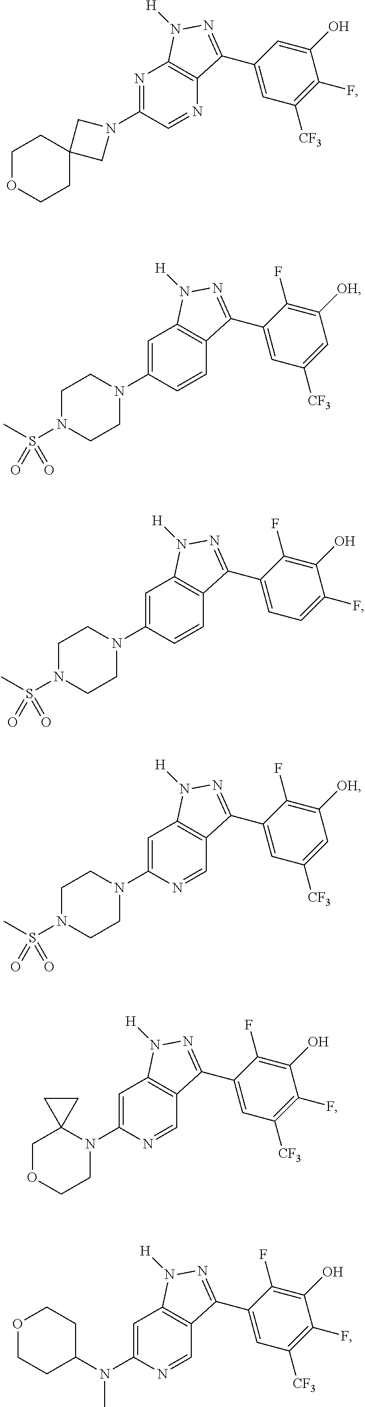 [SMILES: Oc1cc(-c2n[nH]c3nc(N4CC5(CCOCC5)C4)cnc23)cc(C(F)(F)F)c1F ; CS(=O)(=O)N1CCN(c2ccc3c(-c4cc(C(F)(F)F)cc(O)c4F)n[nH]c3c2)CC1 ; CS(=O)(=O)N1CCN(c2ccc3c(-c4ccc(F)c(O)c4F)n[nH]c3c2)CC1 ; CS(=O)(=O)N1CCN(c2cc3[nH]nc(-c4cc(C(F)(F)F)cc(O)c4F)c3cn2)CC1 ; Oc1c(F)c(-c2n[nH]c3cc(N4CCOCC45CC5)ncc23)cc(C(F)(F)F)c1F ; CN(c1cc2[nH]nc(-c3cc(C(F)(F)F)c(F)c(O)c3F)c2cn1)C1CCOCC1] 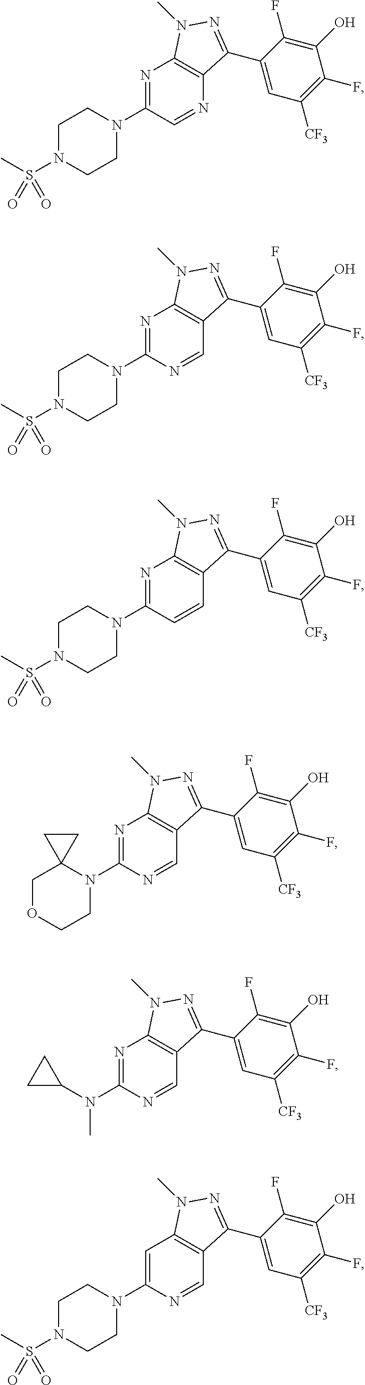 [SMILES: Cn1nc(-c2cc(C(F)(F)F)c(F)c(O)c2F)c2ncc(N3CCN(S(C)(=O)=O)CC3)nc21 ; Cn1nc(-c2cc(C(F)(F)F)c(F)c(O)c2F)c2cnc(N3CCN(S(C)(=O)=O)CC3)nc21 ; Cn1nc(-c2cc(C(F)(F)F)c(F)c(O)c2F)c2ccc(N3CCN(S(C)(=O)=O)CC3)nc21 ; Cn1nc(-c2cc(C(F)(F)F)c(F)c(O)c2F)c2cnc(N3CCOCC34CC4)nc21 ; CN(c1ncc2c(-c3cc(C(F)(F)F)c(F)c(O)c3F)nn(C)c2n1)C1CC1 ; Cn1nc(-c2cc(C(F)(F)F)c(F)c(O)c2F)c2cnc(N3CCN(S(C)(=O)=O)CC3)cc21] 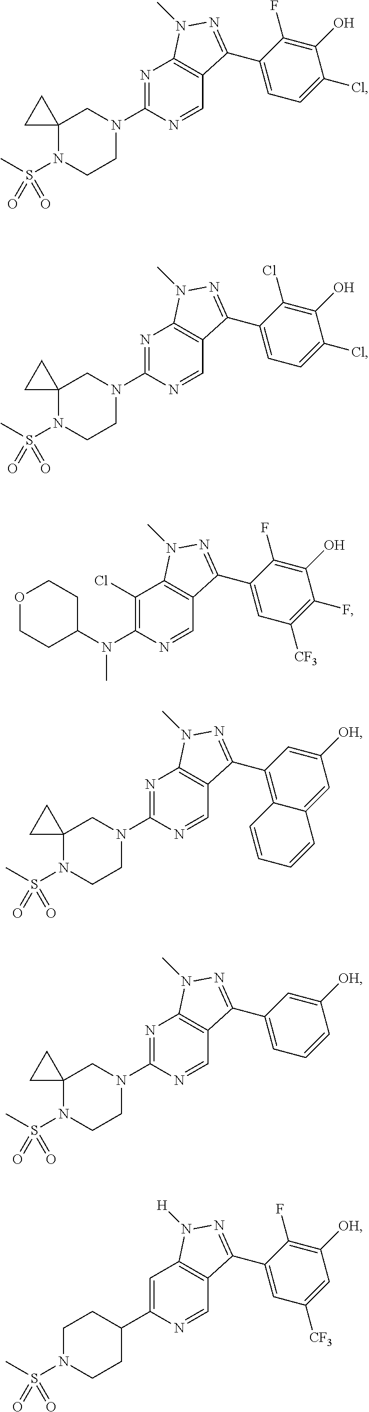 [SMILES: Cn1nc(-c2ccc(Cl)c(O)c2F)c2cnc(N3CCN(S(C)(=O)=O)C4(CC4)C3)nc21 ; Cn1nc(-c2ccc(Cl)c(O)c2Cl)c2cnc(N3CCN(S(C)(=O)=O)C4(CC4)C3)nc21 ; CN(c1ncc2c(-c3cc(C(F)(F)F)c(F)c(O)c3F)nn(C)c2c1Cl)C1CCOCC1 ; Cn1nc(-c2cc(O)cc3ccccc23)c2cnc(N3CCN(S(C)(=O)=O)C4(CC4)C3)nc21 ; Cn1nc(-c2cccc(O)c2)c2cnc(N3CCN(S(C)(=O)=O)C4(CC4)C3)nc21 ; CS(=O)(=O)N1CCC(c2cc3[nH]nc(-c4cc(C(F)(F)F)cc(O)c4F)c3cn2)CC1] 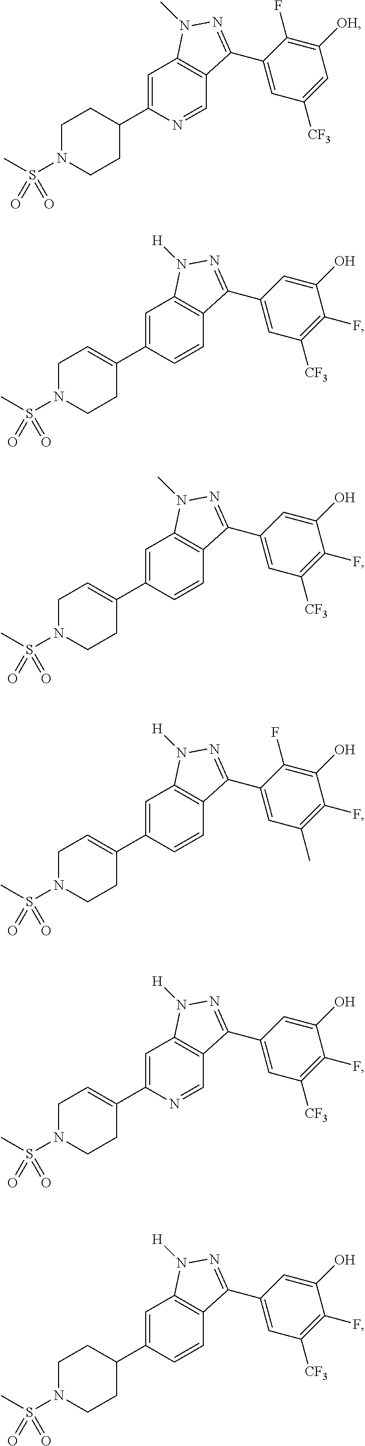 [SMILES: Cn1nc(-c2cc(C(F)(F)F)cc(O)c2F)c2cnc(C3CCN(S(C)(=O)=O)CC3)cc21 ; CS(=O)(=O)N1CC=C(c2ccc3c(-c4cc(O)c(F)c(C(F)(F)F)c4)n[nH]c3c2)CC1 ; Cn1nc(-c2cc(O)c(F)c(C(F)(F)F)c2)c2ccc(C3=CCN(S(C)(=O)=O)CC3)cc21 ; Cc1cc(-c2n[nH]c3cc(C4=CCN(S(C)(=O)=O)CC4)ccc23)c(F)c(O)c1F ; CS(=O)(=O)N1CC=C(c2cc3[nH]nc(-c4cc(O)c(F)c(C(F)(F)F)c4)c3cn2)CC1 ; CS(=O)(=O)N1CCC(c2ccc3c(-c4cc(O)c(F)c(C(F)(F)F)c4)n[nH]c3c2)CC1] 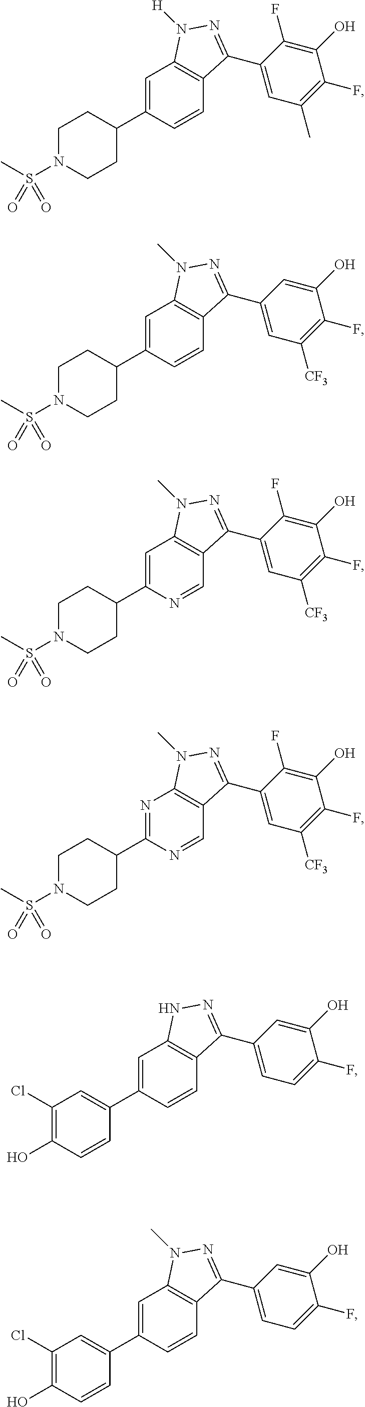 [SMILES: Cc1cc(-c2n[nH]c3cc(C4CCN(S(C)(=O)=O)CC4)ccc23)c(F)c(O)c1F ; Cn1nc(-c2cc(O)c(F)c(C(F)(F)F)c2)c2ccc(C3CCN(S(C)(=O)=O)CC3)cc21 ; Cn1nc(-c2cc(C(F)(F)F)c(F)c(O)c2F)c2cnc(C3CCN(S(C)(=O)=O)CC3)cc21 ; Cn1nc(-c2cc(C(F)(F)F)c(F)c(O)c2F)c2cnc(C3CCN(S(C)(=O)=O)CC3)nc21 ; Oc1cc(-c2n[nH]c3cc(-c4ccc(O)c(Cl)c4)ccc23)ccc1F ; Cn1nc(-c2ccc(F)c(O)c2)c2ccc(-c3ccc(O)c(Cl)c3)cc21] 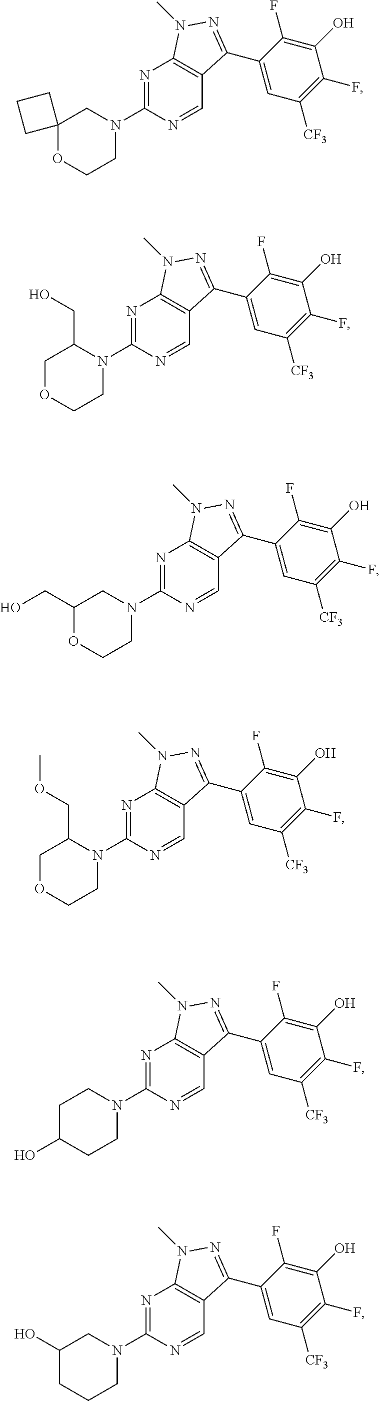 [SMILES: Cn1nc(-c2cc(C(F)(F)F)c(F)c(O)c2F)c2cnc(N3CCOC4(CCC4)C3)nc21 ; Cn1nc(-c2cc(C(F)(F)F)c(F)c(O)c2F)c2cnc(N3CCOCC3CO)nc21 ; Cn1nc(-c2cc(C(F)(F)F)c(F)c(O)c2F)c2cnc(N3CCOC(CO)C3)nc21 ; COCC1COCCN1c1ncc2c(-c3cc(C(F)(F)F)c(F)c(O)c3F)nn(C)c2n1 ; Cn1nc(-c2cc(C(F)(F)F)c(F)c(O)c2F)c2cnc(N3CCC(O)CC3)nc21 ; Cn1nc(-c2cc(C(F)(F)F)c(F)c(O)c2F)c2cnc(N3CCCC(O)C3)nc21] 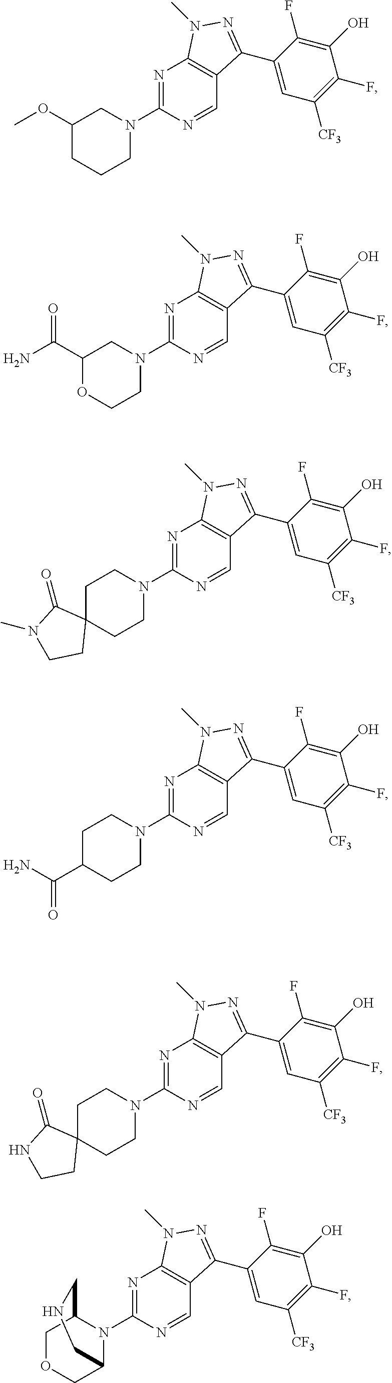 [SMILES: COC1CCCN(c2ncc3c(-c4cc(C(F)(F)F)c(F)c(O)c4F)nn(C)c3n2)C1 ; Cn1nc(-c2cc(C(F)(F)F)c(F)c(O)c2F)c2cnc(N3CCOC(C(N)=O)C3)nc21 ; CN1CCC2(CCN(c3ncc4c(-c5cc(C(F)(F)F)c(F)c(O)c5F)nn(C)c4n3)CC2)C1=O ; Cn1nc(-c2cc(C(F)(F)F)c(F)c(O)c2F)c2cnc(N3CCC(C(N)=O)CC3)nc21 ; Cn1nc(-c2cc(C(F)(F)F)c(F)c(O)c2F)c2cnc(N3CCC4(CCNC4=O)CC3)nc21 ; Cn1nc(-c2cc(C(F)(F)F)c(F)c(O)c2F)c2cnc(N3[C@@H]4CNC[C@H]3COC4)nc21] 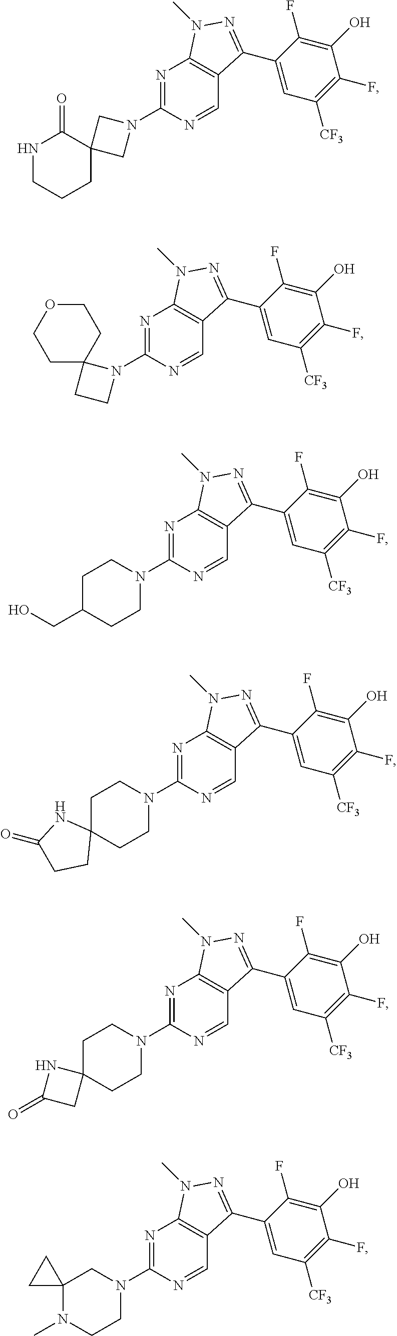 [SMILES: Cn1nc(-c2cc(C(F)(F)F)c(F)c(O)c2F)c2cnc(N3CC4(CCCNC4=O)C3)nc21 ; Cn1nc(-c2cc(C(F)(F)F)c(F)c(O)c2F)c2cnc(N3CCC34CCOCC4)nc21 ; Cn1nc(-c2cc(C(F)(F)F)c(F)c(O)c2F)c2cnc(N3CCC(CO)CC3)nc21 ; Cn1nc(-c2cc(C(F)(F)F)c(F)c(O)c2F)c2cnc(N3CCC4(CCC(=O)N4)CC3)nc21 ; Cn1nc(-c2cc(C(F)(F)F)c(F)c(O)c2F)c2cnc(N3CCC4(CC3)CC(=O)N4)nc21 ; CN1CCN(c2ncc3c(-c4cc(C(F)(F)F)c(F)c(O)c4F)nn(C)c3n2)CC12CC2] 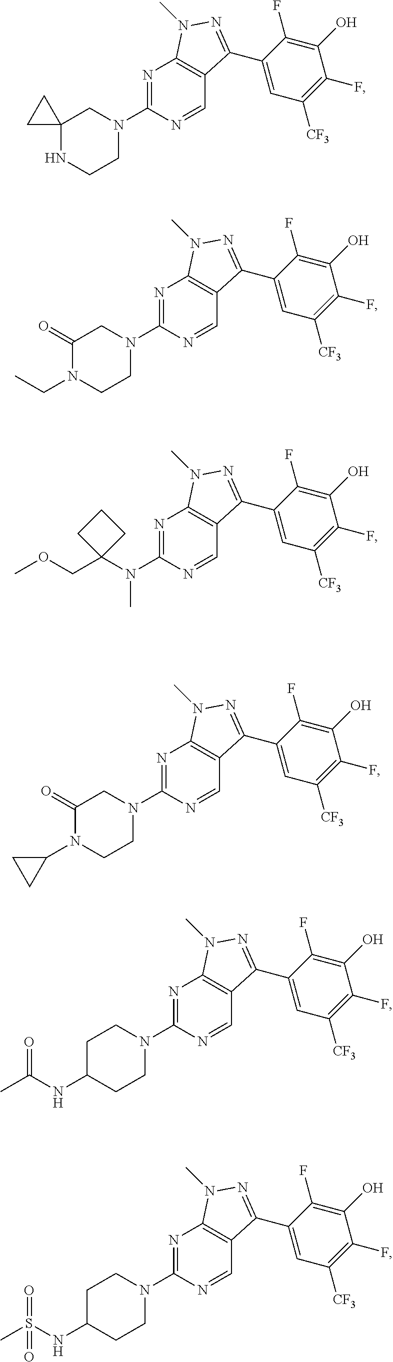 [SMILES: Cn1nc(-c2cc(C(F)(F)F)c(F)c(O)c2F)c2cnc(N3CCNC4(CC4)C3)nc21 ; CCN1CCN(c2ncc3c(-c4cc(C(F)(F)F)c(F)c(O)c4F)nn(C)c3n2)CC1=O ; COCC1(N(C)c2ncc3c(-c4cc(C(F)(F)F)c(F)c(O)c4F)nn(C)c3n2)CCC1 ; Cn1nc(-c2cc(C(F)(F)F)c(F)c(O)c2F)c2cnc(N3CCN(C4CC4)C(=O)C3)nc21 ; CC(=O)NC1CCN(c2ncc3c(-c4cc(C(F)(F)F)c(F)c(O)c4F)nn(C)c3n2)CC1 ; Cn1nc(-c2cc(C(F)(F)F)c(F)c(O)c2F)c2cnc(N3CCC(NS(C)(=O)=O)CC3)nc21] 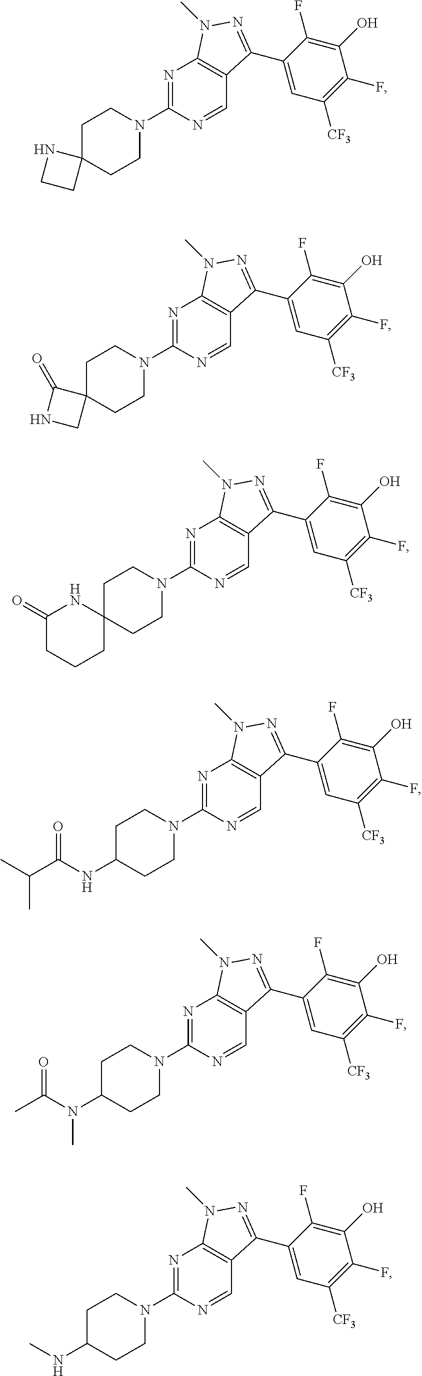 [SMILES: Cn1nc(-c2cc(C(F)(F)F)c(F)c(O)c2F)c2cnc(N3CCC4(CCN4)CC3)nc21 ; Cn1nc(-c2cc(C(F)(F)F)c(F)c(O)c2F)c2cnc(N3CCC4(CC3)CNC4=O)nc21 ; Cn1nc(-c2cc(C(F)(F)F)c(F)c(O)c2F)c2cnc(N3CCC4(CCCC(=O)N4)CC3)nc21 ; CC(C)C(=O)NC1CCN(c2ncc3c(-c4cc(C(F)(F)F)c(F)c(O)c4F)nn(C)c3n2)CC1 ; CC(=O)N(C)C1CCN(c2ncc3c(-c4cc(C(F)(F)F)c(F)c(O)c4F)nn(C)c3n2)CC1 ; CNC1CCN(c2ncc3c(-c4cc(C(F)(F)F)c(F)c(O)c4F)nn(C)c3n2)CC1] 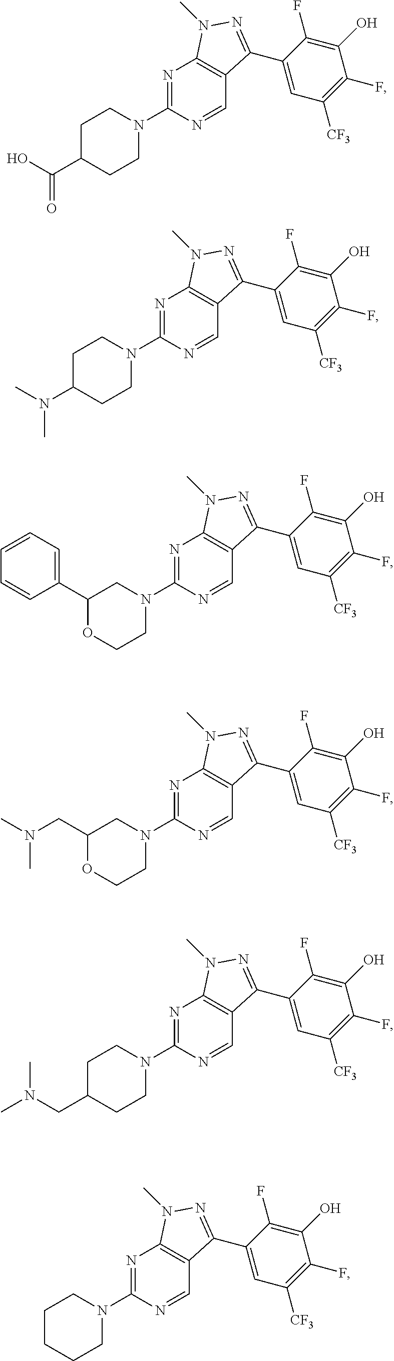 [SMILES: Cn1nc(-c2cc(C(F)(F)F)c(F)c(O)c2F)c2cnc(N3CCC(C(=O)O)CC3)nc21 ; CN(C)C1CCN(c2ncc3c(-c4cc(C(F)(F)F)c(F)c(O)c4F)nn(C)c3n2)CC1 ; Cn1nc(-c2cc(C(F)(F)F)c(F)c(O)c2F)c2cnc(N3CCOC(c4ccccc4)C3)nc21 ; CN(C)CC1CN(c2ncc3c(-c4cc(C(F)(F)F)c(F)c(O)c4F)nn(C)c3n2)CCO1 ; CN(C)CC1CCN(c2ncc3c(-c4cc(C(F)(F)F)c(F)c(O)c4F)nn(C)c3n2)CC1 ; Cn1nc(-c2cc(C(F)(F)F)c(F)c(O)c2F)c2cnc(N3CCCCC3)nc21] 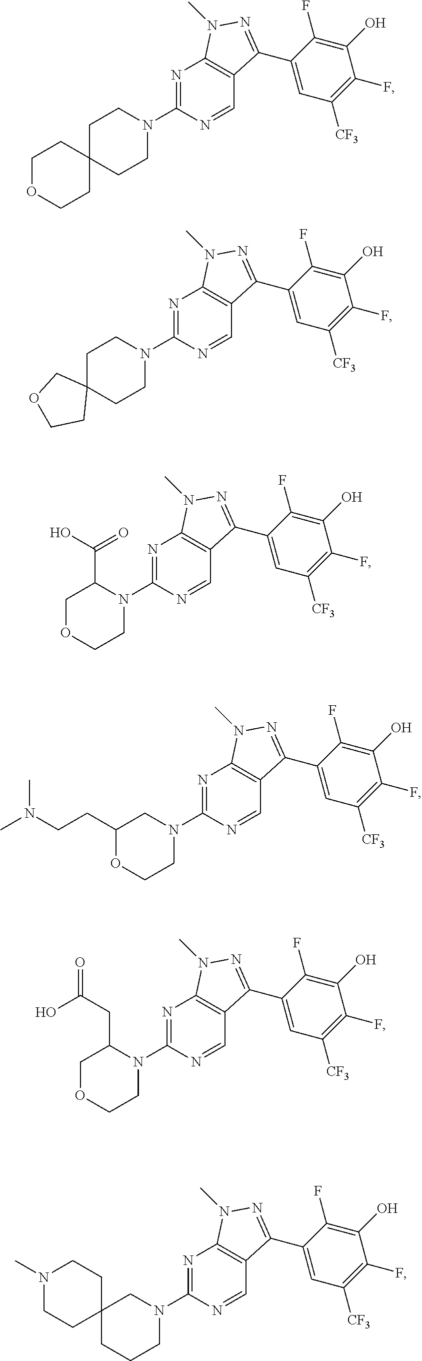 [SMILES: Cn1nc(-c2cc(C(F)(F)F)c(F)c(O)c2F)c2cnc(N3CCC4(CCOCC4)CC3)nc21 ; Cn1nc(-c2cc(C(F)(F)F)c(F)c(O)c2F)c2cnc(N3CCC4(CCOC4)CC3)nc21 ; Cn1nc(-c2cc(C(F)(F)F)c(F)c(O)c2F)c2cnc(N3CCOCC3C(=O)O)nc21 ; CN(C)CCC1CN(c2ncc3c(-c4cc(C(F)(F)F)c(F)c(O)c4F)nn(C)c3n2)CCO1 ; Cn1nc(-c2cc(C(F)(F)F)c(F)c(O)c2F)c2cnc(N3CCOCC3CC(=O)O)nc21 ; CN1CCC2(CCCN(c3ncc4c(-c5cc(C(F)(F)F)c(F)c(O)c5F)nn(C)c4n3)C2)CC1] 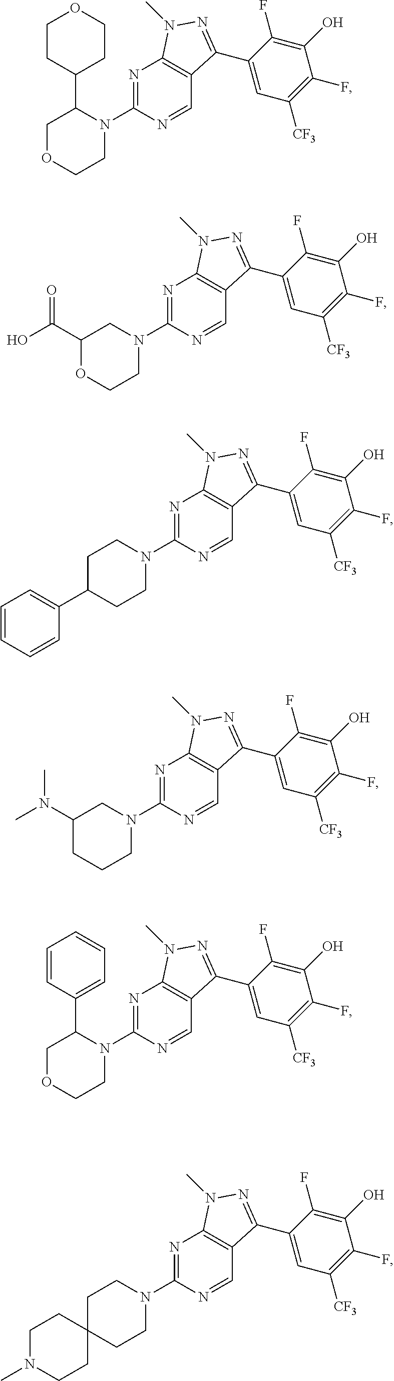 [SMILES: Cn1nc(-c2cc(C(F)(F)F)c(F)c(O)c2F)c2cnc(N3CCOCC3C3CCOCC3)nc21 ; Cn1nc(-c2cc(C(F)(F)F)c(F)c(O)c2F)c2cnc(N3CCOC(C(=O)O)C3)nc21 ; Cn1nc(-c2cc(C(F)(F)F)c(F)c(O)c2F)c2cnc(N3CCC(c4ccccc4)CC3)nc21 ; CN(C)C1CCCN(c2ncc3c(-c4cc(C(F)(F)F)c(F)c(O)c4F)nn(C)c3n2)C1 ; Cn1nc(-c2cc(C(F)(F)F)c(F)c(O)c2F)c2cnc(N3CCOCC3c3ccccc3)nc21 ; CN1CCC2(CC1)CCN(c1ncc3c(-c4cc(C(F)(F)F)c(F)c(O)c4F)nn(C)c3n1)CC2] 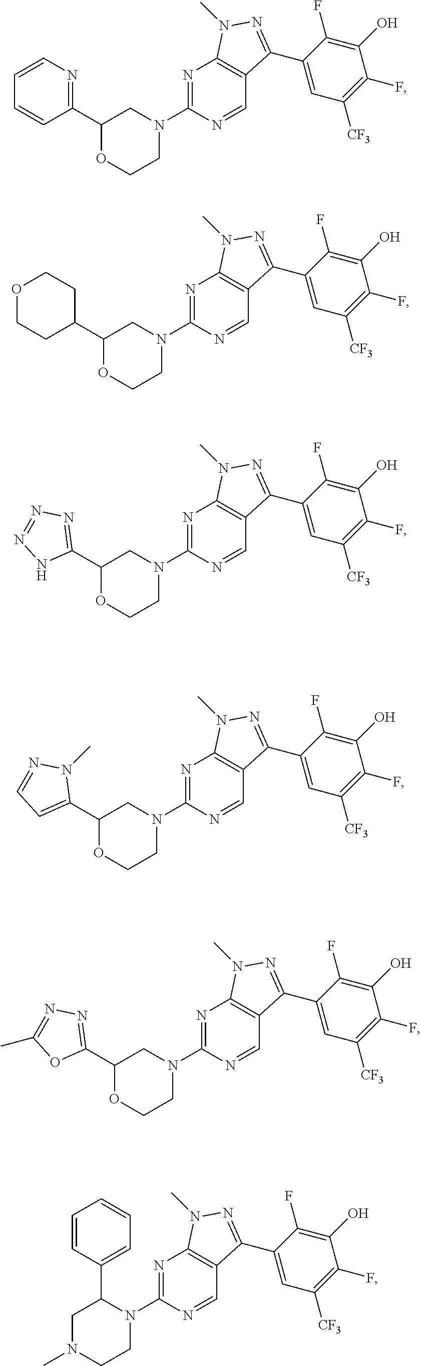 [SMILES: Cn1nc(-c2cc(C(F)(F)F)c(F)c(O)c2F)c2cnc(N3CCOC(c4ccccn4)C3)nc21 ; Cn1nc(-c2cc(C(F)(F)F)c(F)c(O)c2F)c2cnc(N3CCOC(C4CCOCC4)C3)nc21 ; Cn1nc(-c2cc(C(F)(F)F)c(F)c(O)c2F)c2cnc(N3CCOC(c4nnn[nH]4)C3)nc21 ; Cn1nccc1C1CN(c2ncc3c(-c4cc(C(F)(F)F)c(F)c(O)c4F)nn(C)c3n2)CCO1 ; Cc1nnc(C2CN(c3ncc4c(-c5cc(C(F)(F)F)c(F)c(O)c5F)nn(C)c4n3)CCO2)o1 ; CN1CCN(c2ncc3c(-c4cc(C(F)(F)F)c(F)c(O)c4F)nn(C)c3n2)C(c2ccccc2)C1] 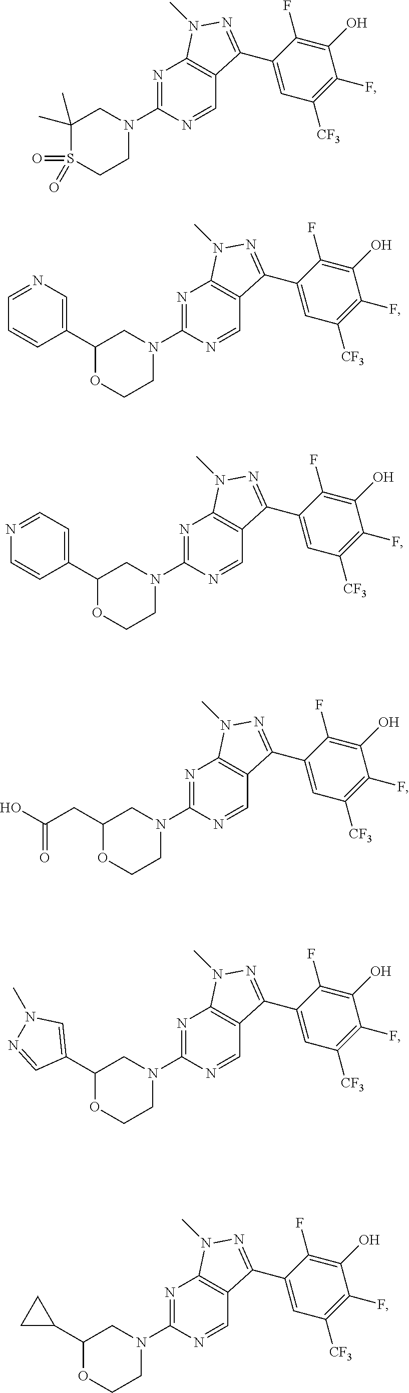 [SMILES: Cn1nc(-c2cc(C(F)(F)F)c(F)c(O)c2F)c2cnc(N3CCS(=O)(=O)C(C)(C)C3)nc21 ; Cn1nc(-c2cc(C(F)(F)F)c(F)c(O)c2F)c2cnc(N3CCOC(c4cccnc4)C3)nc21 ; Cn1nc(-c2cc(C(F)(F)F)c(F)c(O)c2F)c2cnc(N3CCOC(c4ccncc4)C3)nc21 ; Cn1nc(-c2cc(C(F)(F)F)c(F)c(O)c2F)c2cnc(N3CCOC(CC(=O)O)C3)nc21 ; Cn1cc(C2CN(c3ncc4c(-c5cc(C(F)(F)F)c(F)c(O)c5F)nn(C)c4n3)CCO2)cn1 ; Cn1nc(-c2cc(C(F)(F)F)c(F)c(O)c2F)c2cnc(N3CCOC(C4CC4)C3)nc21] 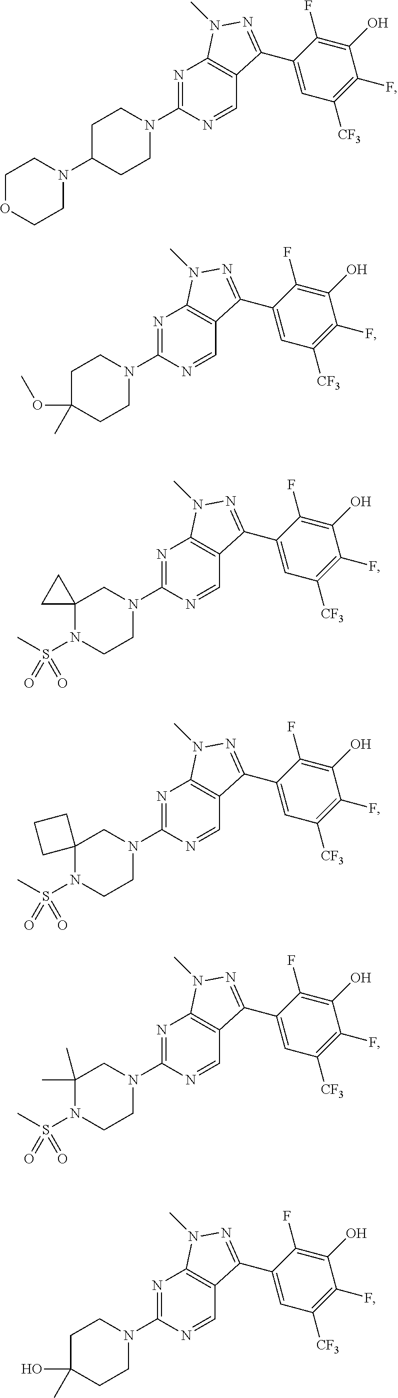 [SMILES: Cn1nc(-c2cc(C(F)(F)F)c(F)c(O)c2F)c2cnc(N3CCC(N4CCOCC4)CC3)nc21 ; COC1(C)CCN(c2ncc3c(-c4cc(C(F)(F)F)c(F)c(O)c4F)nn(C)c3n2)CC1 ; Cn1nc(-c2cc(C(F)(F)F)c(F)c(O)c2F)c2cnc(N3CCN(S(C)(=O)=O)C4(CC4)C3)nc21 ; Cn1nc(-c2cc(C(F)(F)F)c(F)c(O)c2F)c2cnc(N3CCN(S(C)(=O)=O)C4(CCC4)C3)nc21 ; Cn1nc(-c2cc(C(F)(F)F)c(F)c(O)c2F)c2cnc(N3CCN(S(C)(=O)=O)C(C)(C)C3)nc21 ; Cn1nc(-c2cc(C(F)(F)F)c(F)c(O)c2F)c2cnc(N3CCC(C)(O)CC3)nc21] 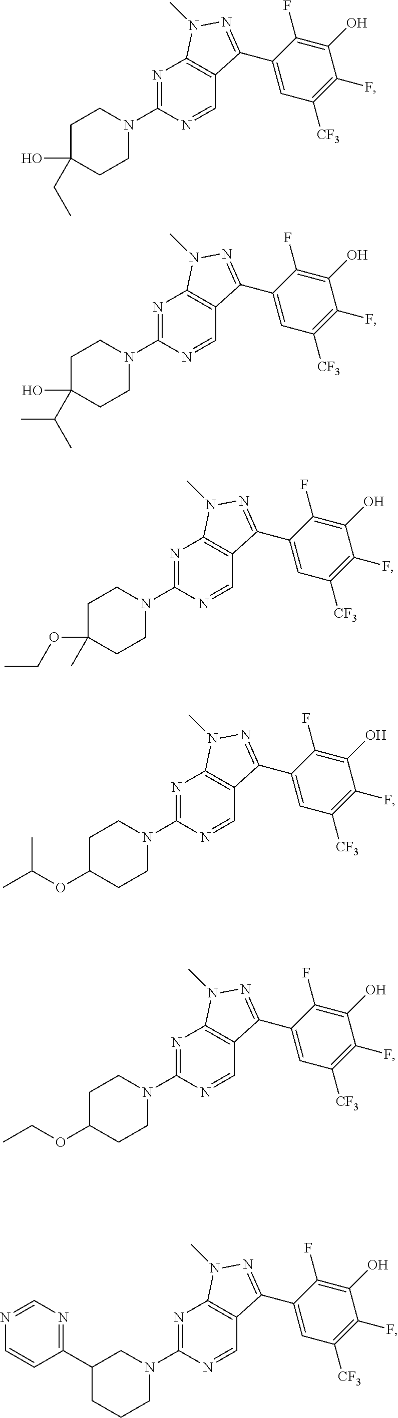 [SMILES: CCC1(O)CCN(c2ncc3c(-c4cc(C(F)(F)F)c(F)c(O)c4F)nn(C)c3n2)CC1 ; CC(C)C1(O)CCN(c2ncc3c(-c4cc(C(F)(F)F)c(F)c(O)c4F)nn(C)c3n2)CC1 ; CCOC1(C)CCN(c2ncc3c(-c4cc(C(F)(F)F)c(F)c(O)c4F)nn(C)c3n2)CC1 ; CC(C)OC1CCN(c2ncc3c(-c4cc(C(F)(F)F)c(F)c(O)c4F)nn(C)c3n2)CC1 ; CCOC1CCN(c2ncc3c(-c4cc(C(F)(F)F)c(F)c(O)c4F)nn(C)c3n2)CC1 ; Cn1nc(-c2cc(C(F)(F)F)c(F)c(O)c2F)c2cnc(N3CCCC(c4ccncn4)C3)nc21] 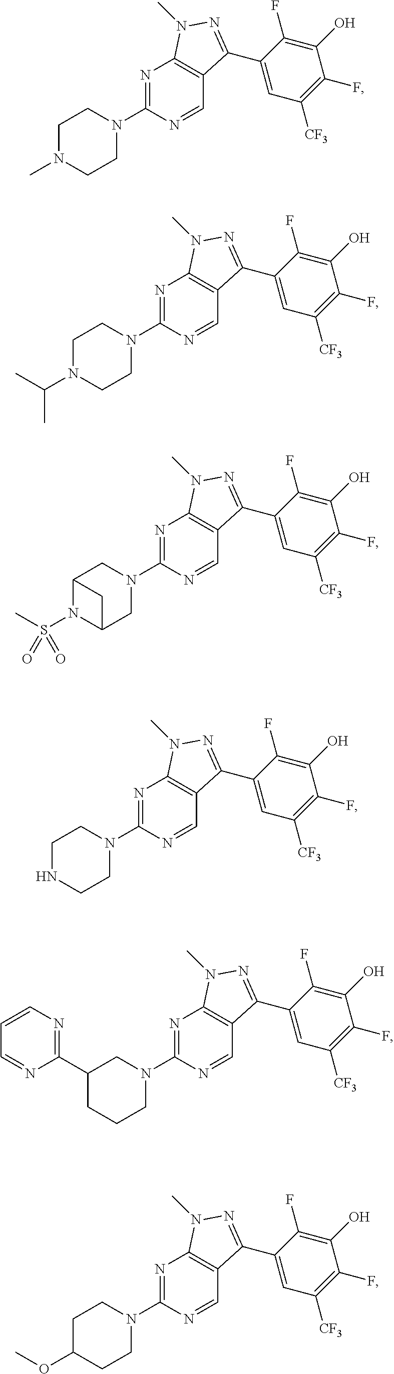 [SMILES: CN1CCN(c2ncc3c(-c4cc(C(F)(F)F)c(F)c(O)c4F)nn(C)c3n2)CC1 ; CC(C)N1CCN(c2ncc3c(-c4cc(C(F)(F)F)c(F)c(O)c4F)nn(C)c3n2)CC1 ; Cn1nc(-c2cc(C(F)(F)F)c(F)c(O)c2F)c2cnc(N3CC4CC(C3)N4S(C)(=O)=O)nc21 ; Cn1nc(-c2cc(C(F)(F)F)c(F)c(O)c2F)c2cnc(N3CCNCC3)nc21 ; Cn1nc(-c2cc(C(F)(F)F)c(F)c(O)c2F)c2cnc(N3CCCC(c4ncccn4)C3)nc21 ; COC1CCN(c2ncc3c(-c4cc(C(F)(F)F)c(F)c(O)c4F)nn(C)c3n2)CC1] 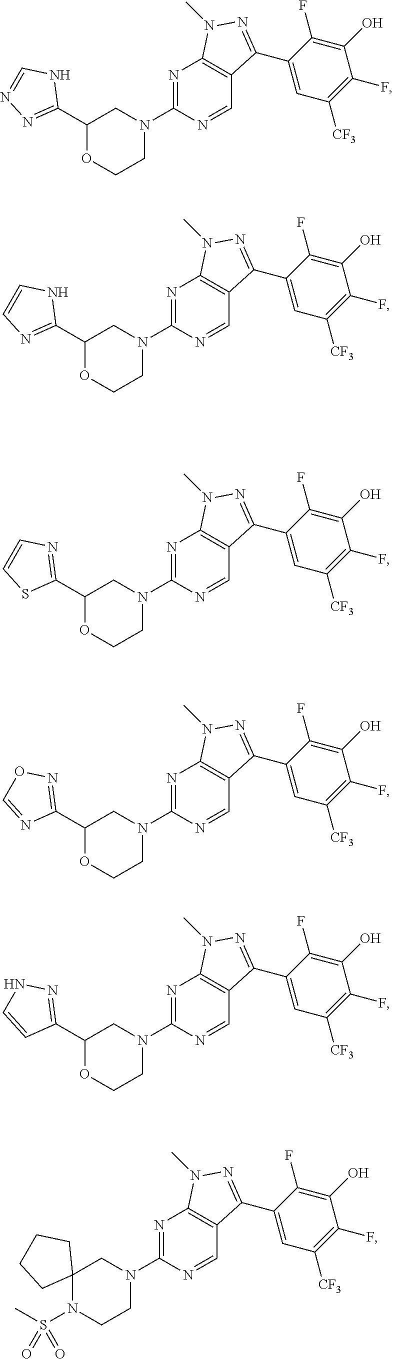 [SMILES: Cn1nc(-c2cc(C(F)(F)F)c(F)c(O)c2F)c2cnc(N3CCOC(c4nnc[nH]4)C3)nc21 ; Cn1nc(-c2cc(C(F)(F)F)c(F)c(O)c2F)c2cnc(N3CCOC(c4ncc[nH]4)C3)nc21 ; Cn1nc(-c2cc(C(F)(F)F)c(F)c(O)c2F)c2cnc(N3CCOC(c4nccs4)C3)nc21 ; Cn1nc(-c2cc(C(F)(F)F)c(F)c(O)c2F)c2cnc(N3CCOC(c4ncon4)C3)nc21 ; Cn1nc(-c2cc(C(F)(F)F)c(F)c(O)c2F)c2cnc(N3CCOC(c4cc[nH]n4)C3)nc21 ; Cn1nc(-c2cc(C(F)(F)F)c(F)c(O)c2F)c2cnc(N3CCN(S(C)(=O)=O)C4(CCCC4)C3)nc21] 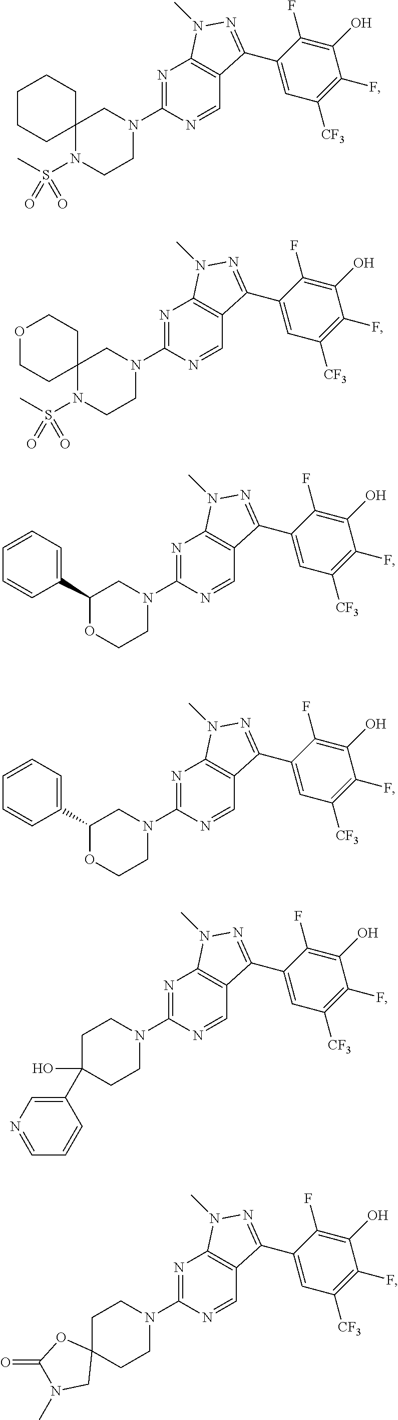 [SMILES: Cn1nc(-c2cc(C(F)(F)F)c(F)c(O)c2F)c2cnc(N3CCN(S(C)(=O)=O)C4(CCCCC4)C3)nc21 ; Cn1nc(-c2cc(C(F)(F)F)c(F)c(O)c2F)c2cnc(N3CCN(S(C)(=O)=O)C4(CCOCC4)C3)nc21 ; Cn1nc(-c2cc(C(F)(F)F)c(F)c(O)c2F)c2cnc(N3CCO[C@@H](c4ccccc4)C3)nc21 ; Cn1nc(-c2cc(C(F)(F)F)c(F)c(O)c2F)c2cnc(N3CCO[C@H](c4ccccc4)C3)nc21 ; Cn1nc(-c2cc(C(F)(F)F)c(F)c(O)c2F)c2cnc(N3CCC(O)(c4cccnc4)CC3)nc21 ; CN1CC2(CCN(c3ncc4c(-c5cc(C(F)(F)F)c(F)c(O)c5F)nn(C)c4n3)CC2)OC1=O] 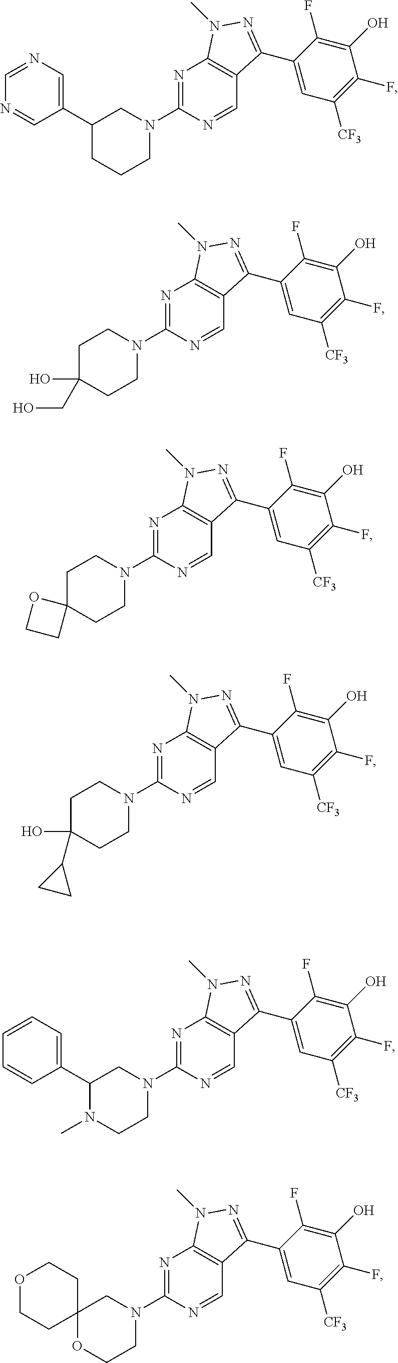 [SMILES: Cn1nc(-c2cc(C(F)(F)F)c(F)c(O)c2F)c2cnc(N3CCCC(c4cncnc4)C3)nc21 ; Cn1nc(-c2cc(C(F)(F)F)c(F)c(O)c2F)c2cnc(N3CCC(O)(CO)CC3)nc21 ; Cn1nc(-c2cc(C(F)(F)F)c(F)c(O)c2F)c2cnc(N3CCC4(CCO4)CC3)nc21 ; Cn1nc(-c2cc(C(F)(F)F)c(F)c(O)c2F)c2cnc(N3CCC(O)(C4CC4)CC3)nc21 ; CN1CCN(c2ncc3c(-c4cc(C(F)(F)F)c(F)c(O)c4F)nn(C)c3n2)CC1c1ccccc1 ; Cn1nc(-c2cc(C(F)(F)F)c(F)c(O)c2F)c2cnc(N3CCOC4(CCOCC4)C3)nc21] 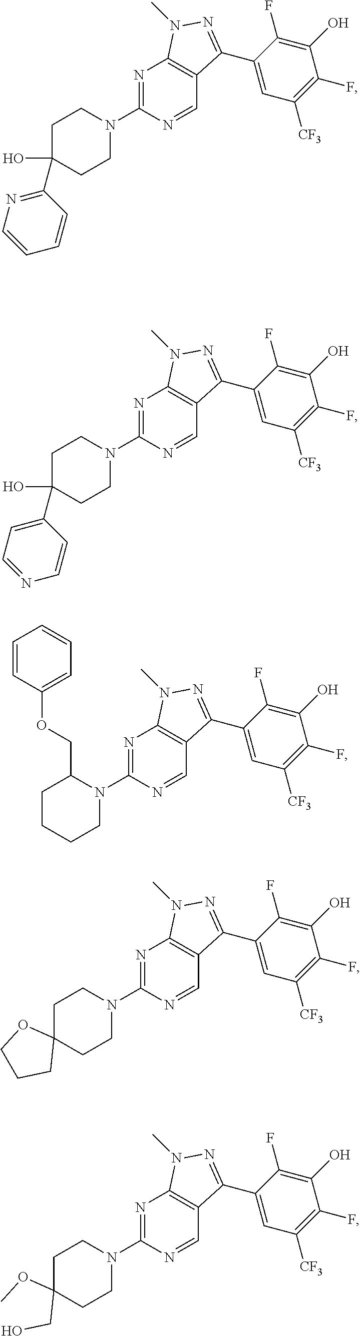 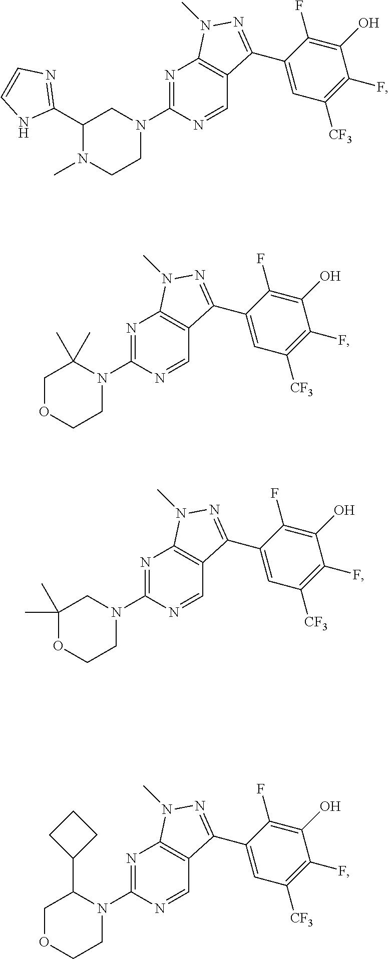 [SMILES: CN1CCN(c2ncc3c(-c4cc(C(F)(F)F)c(F)c(O)c4F)nn(C)c3n2)CC1c1ncc[nH]1 ; Cn1nc(-c2cc(C(F)(F)F)c(F)c(O)c2F)c2cnc(N3CCOCC3(C)C)nc21 ; Cn1nc(-c2cc(C(F)(F)F)c(F)c(O)c2F)c2cnc(N3CCOC(C)(C)C3)nc21 ; Cn1nc(-c2cc(C(F)(F)F)c(F)c(O)c2F)c2cnc(N3CCOCC3C3CCC3)nc21] 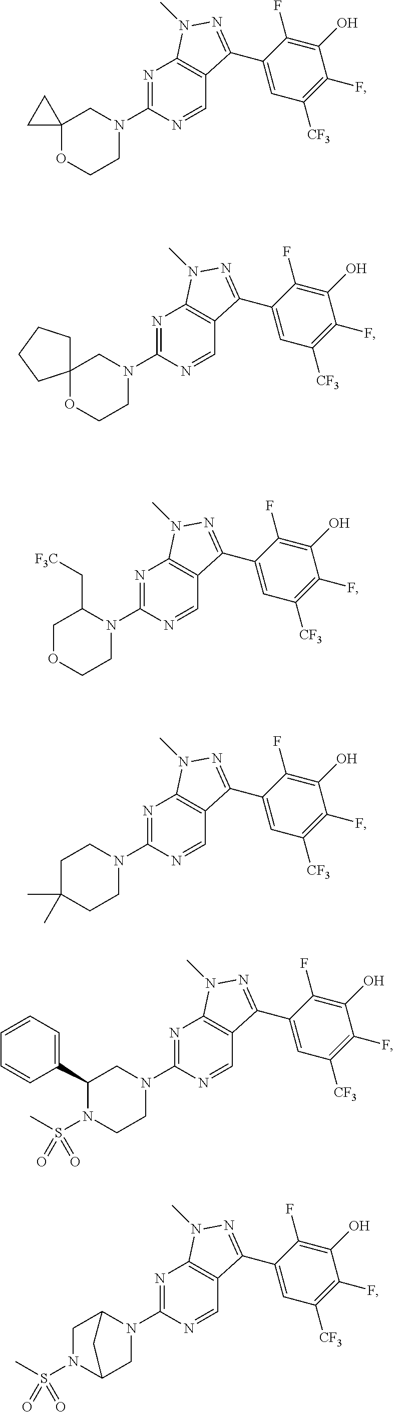 [SMILES: Cn1nc(-c2cc(C(F)(F)F)c(F)c(O)c2F)c2cnc(N3CCOC4(CC4)C3)nc21 ; Cn1nc(-c2cc(C(F)(F)F)c(F)c(O)c2F)c2cnc(N3CCOC4(CCCC4)C3)nc21 ; Cn1nc(-c2cc(C(F)(F)F)c(F)c(O)c2F)c2cnc(N3CCOCC3CC(F)(F)F)nc21 ; Cn1nc(-c2cc(C(F)(F)F)c(F)c(O)c2F)c2cnc(N3CCC(C)(C)CC3)nc21 ; Cn1nc(-c2cc(C(F)(F)F)c(F)c(O)c2F)c2cnc(N3CCN(S(C)(=O)=O)[C@@H](c4ccccc4)C3)nc21 ; Cn1nc(-c2cc(C(F)(F)F)c(F)c(O)c2F)c2cnc(N3CC4CC3CN4S(C)(=O)=O)nc21] 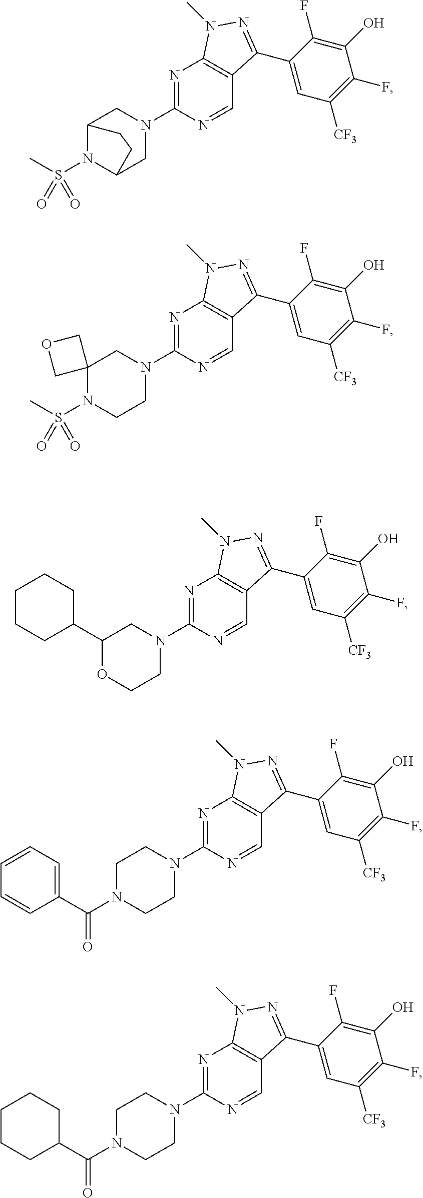 [SMILES: Cn1nc(-c2cc(C(F)(F)F)c(F)c(O)c2F)c2cnc(N3CC4CCC(C3)N4S(C)(=O)=O)nc21 ; Cn1nc(-c2cc(C(F)(F)F)c(F)c(O)c2F)c2cnc(N3CCN(S(C)(=O)=O)C4(COC4)C3)nc21 ; Cn1nc(-c2cc(C(F)(F)F)c(F)c(O)c2F)c2cnc(N3CCOC(C4CCCCC4)C3)nc21 ; Cn1nc(-c2cc(C(F)(F)F)c(F)c(O)c2F)c2cnc(N3CCN(C(=O)c4ccccc4)CC3)nc21 ; Cn1nc(-c2cc(C(F)(F)F)c(F)c(O)c2F)c2cnc(N3CCN(C(=O)C4CCCCC4)CC3)nc21] 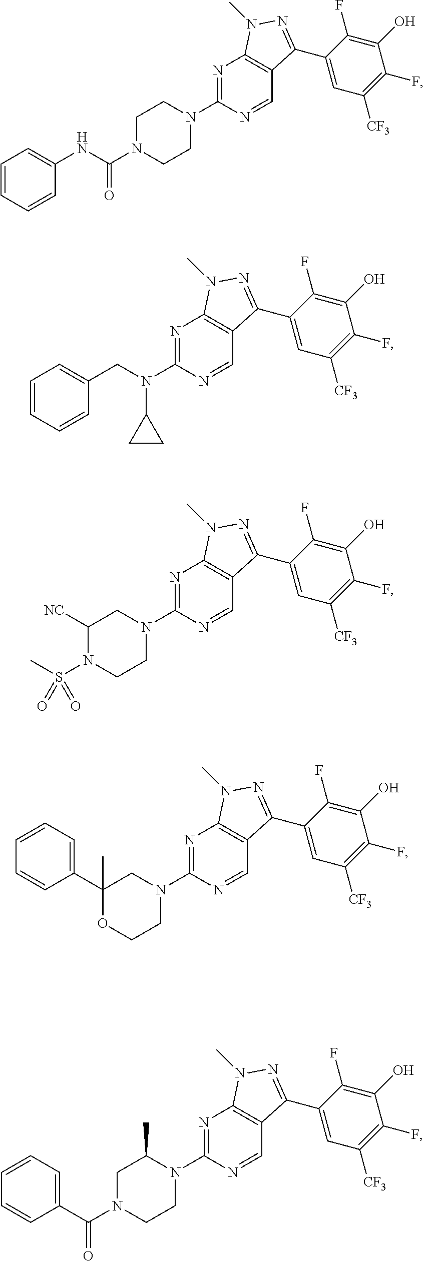 [SMILES: Cn1nc(-c2cc(C(F)(F)F)c(F)c(O)c2F)c2cnc(N3CCN(C(=O)Nc4ccccc4)CC3)nc21 ; Cn1nc(-c2cc(C(F)(F)F)c(F)c(O)c2F)c2cnc(N(Cc3ccccc3)C3CC3)nc21 ; Cn1nc(-c2cc(C(F)(F)F)c(F)c(O)c2F)c2cnc(N3CCN(S(C)(=O)=O)C(C#N)C3)nc21 ; Cn1nc(-c2cc(C(F)(F)F)c(F)c(O)c2F)c2cnc(N3CCOC(C)(c4ccccc4)C3)nc21 ; C[C@@H]1CN(C(=O)c2ccccc2)CCN1c1ncc2c(-c3cc(C(F)(F)F)c(F)c(O)c3F)nn(C)c2n1] 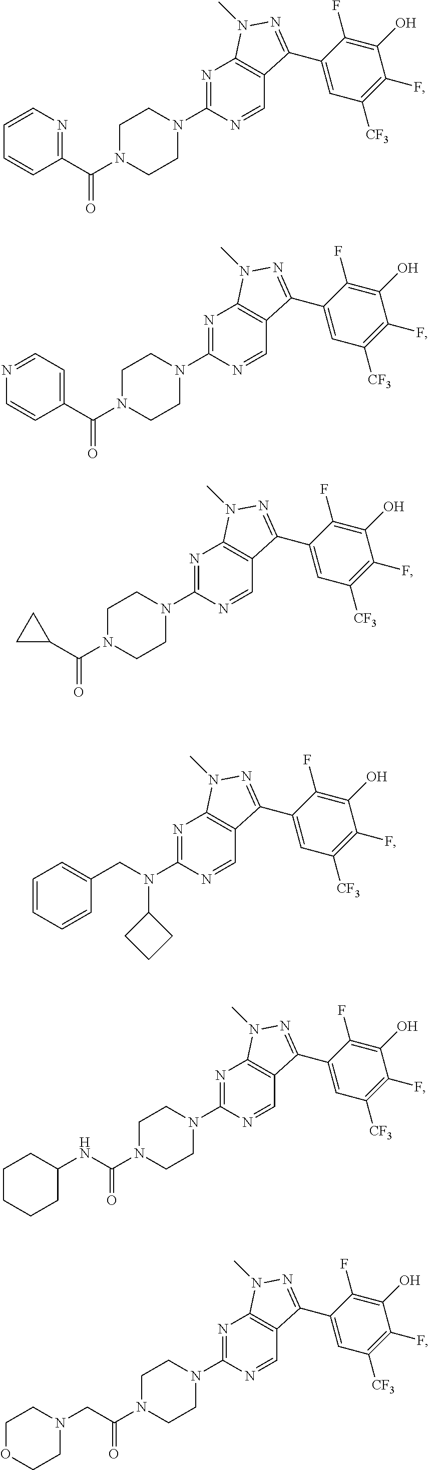 [SMILES: Cn1nc(-c2cc(C(F)(F)F)c(F)c(O)c2F)c2cnc(N3CCN(C(=O)c4ccccn4)CC3)nc21 ; Cn1nc(-c2cc(C(F)(F)F)c(F)c(O)c2F)c2cnc(N3CCN(C(=O)c4ccncc4)CC3)nc21 ; Cn1nc(-c2cc(C(F)(F)F)c(F)c(O)c2F)c2cnc(N3CCN(C(=O)C4CC4)CC3)nc21 ; Cn1nc(-c2cc(C(F)(F)F)c(F)c(O)c2F)c2cnc(N(Cc3ccccc3)C3CCC3)nc21 ; Cn1nc(-c2cc(C(F)(F)F)c(F)c(O)c2F)c2cnc(N3CCN(C(=O)NC4CCCCC4)CC3)nc21 ; Cn1nc(-c2cc(C(F)(F)F)c(F)c(O)c2F)c2cnc(N3CCN(C(=O)CN4CCOCC4)CC3)nc21] 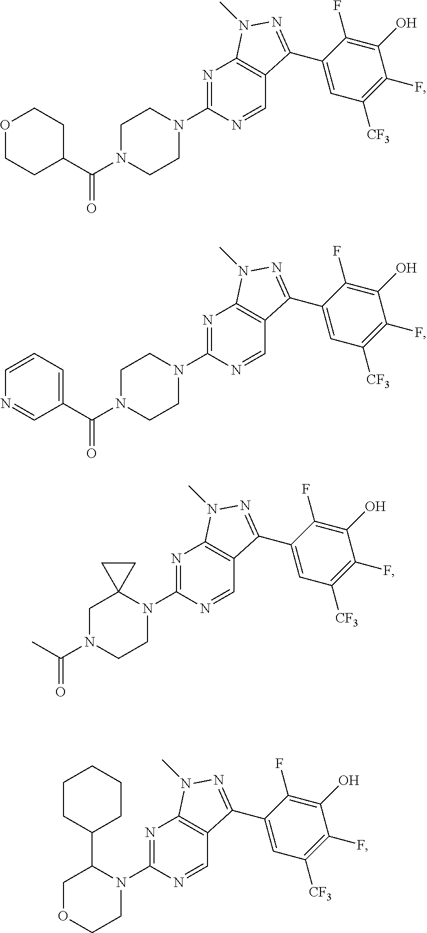 [SMILES: Cn1nc(-c2cc(C(F)(F)F)c(F)c(O)c2F)c2cnc(N3CCN(C(=O)C4CCOCC4)CC3)nc21 ; Cn1nc(-c2cc(C(F)(F)F)c(F)c(O)c2F)c2cnc(N3CCN(C(=O)c4cccnc4)CC3)nc21 ; CC(=O)N1CCN(c2ncc3c(-c4cc(C(F)(F)F)c(F)c(O)c4F)nn(C)c3n2)C2(CC2)C1 ; Cn1nc(-c2cc(C(F)(F)F)c(F)c(O)c2F)c2cnc(N3CCOCC3C3CCCCC3)nc21] 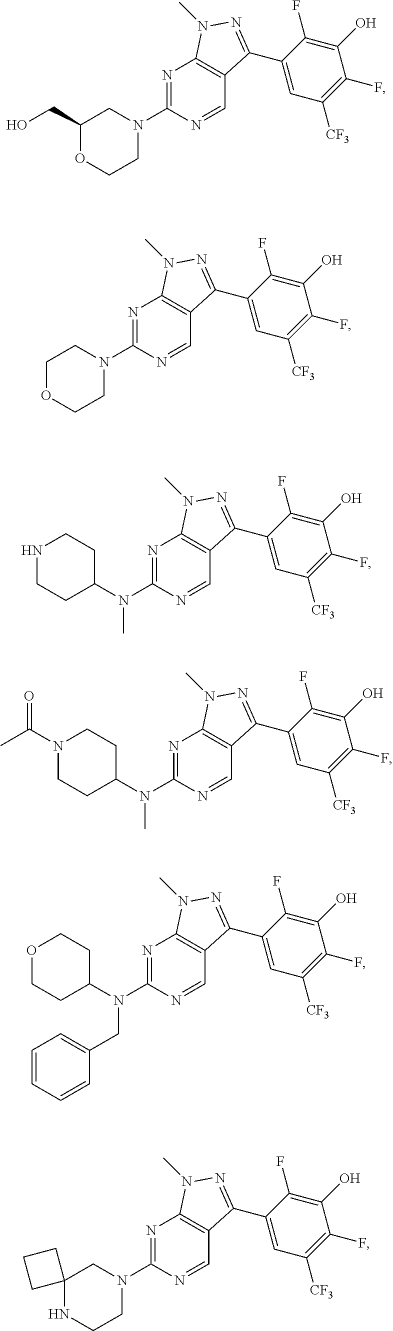 [SMILES: Cn1nc(-c2cc(C(F)(F)F)c(F)c(O)c2F)c2cnc(N3CCO[C@@H](CO)C3)nc21 ; Cn1nc(-c2cc(C(F)(F)F)c(F)c(O)c2F)c2cnc(N3CCOCC3)nc21 ; CN(c1ncc2c(-c3cc(C(F)(F)F)c(F)c(O)c3F)nn(C)c2n1)C1CCNCC1 ; CC(=O)N1CCC(N(C)c2ncc3c(-c4cc(C(F)(F)F)c(F)c(O)c4F)nn(C)c3n2)CC1 ; Cn1nc(-c2cc(C(F)(F)F)c(F)c(O)c2F)c2cnc(N(Cc3ccccc3)C3CCOCC3)nc21 ; Cn1nc(-c2cc(C(F)(F)F)c(F)c(O)c2F)c2cnc(N3CCNC4(CCC4)C3)nc21] 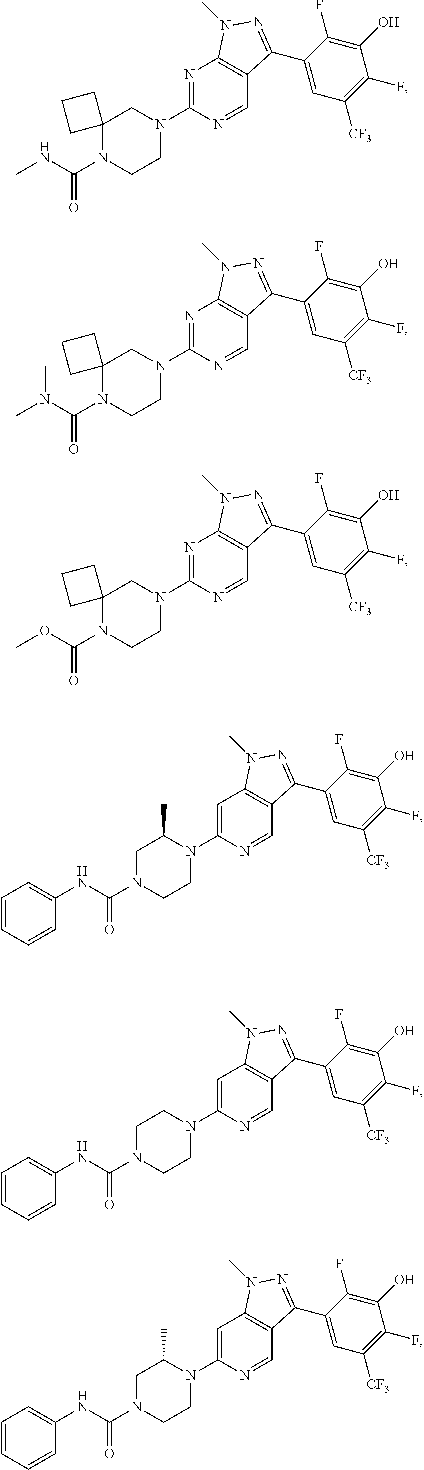 [SMILES: CNC(=O)N1CCN(c2ncc3c(-c4cc(C(F)(F)F)c(F)c(O)c4F)nn(C)c3n2)CC12CCC2 ; CN(C)C(=O)N1CCN(c2ncc3c(-c4cc(C(F)(F)F)c(F)c(O)c4F)nn(C)c3n2)CC12CCC2 ; COC(=O)N1CCN(c2ncc3c(-c4cc(C(F)(F)F)c(F)c(O)c4F)nn(C)c3n2)CC12CCC2 ; C[C@@H]1CN(C(=O)Nc2ccccc2)CCN1c1cc2c(cn1)c(-c1cc(C(F)(F)F)c(F)c(O)c1F)nn2C ; Cn1nc(-c2cc(C(F)(F)F)c(F)c(O)c2F)c2cnc(N3CCN(C(=O)Nc4ccccc4)CC3)cc21 ; C[C@H]1CN(C(=O)Nc2ccccc2)CCN1c1cc2c(cn1)c(-c1cc(C(F)(F)F)c(F)c(O)c1F)nn2C] 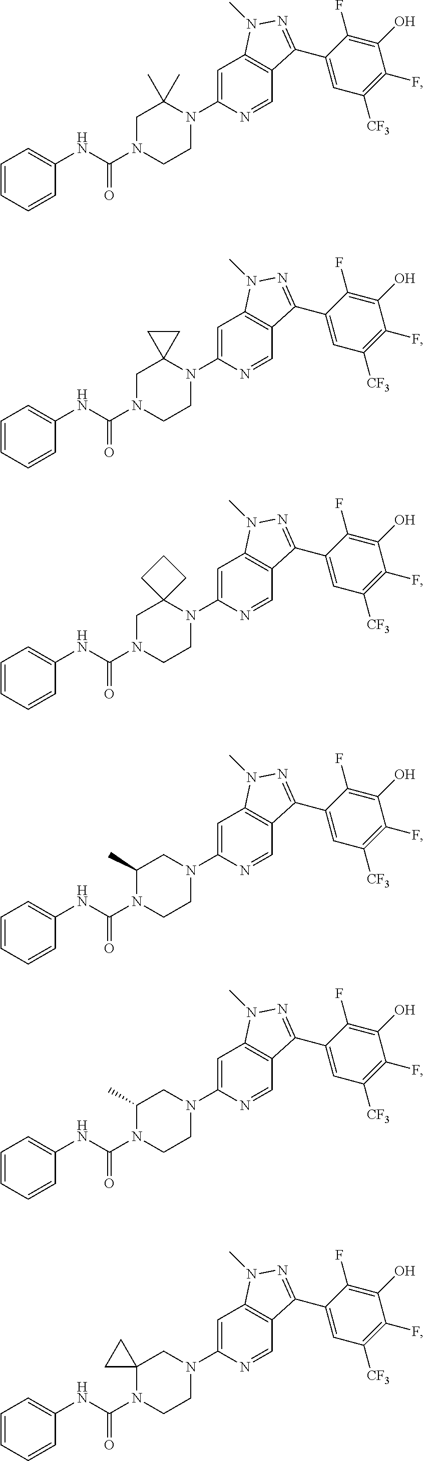 [SMILES: Cn1nc(-c2cc(C(F)(F)F)c(F)c(O)c2F)c2cnc(N3CCN(C(=O)Nc4ccccc4)CC3(C)C)cc21 ; Cn1nc(-c2cc(C(F)(F)F)c(F)c(O)c2F)c2cnc(N3CCN(C(=O)Nc4ccccc4)CC34CC4)cc21 ; Cn1nc(-c2cc(C(F)(F)F)c(F)c(O)c2F)c2cnc(N3CCN(C(=O)Nc4ccccc4)CC34CCC4)cc21 ; C[C@H]1CN(c2cc3c(cn2)c(-c2cc(C(F)(F)F)c(F)c(O)c2F)nn3C)CCN1C(=O)Nc1ccccc1 ; C[C@@H]1CN(c2cc3c(cn2)c(-c2cc(C(F)(F)F)c(F)c(O)c2F)nn3C)CCN1C(=O)Nc1ccccc1 ; Cn1nc(-c2cc(C(F)(F)F)c(F)c(O)c2F)c2cnc(N3CCN(C(=O)Nc4ccccc4)C4(CC4)C3)cc21] 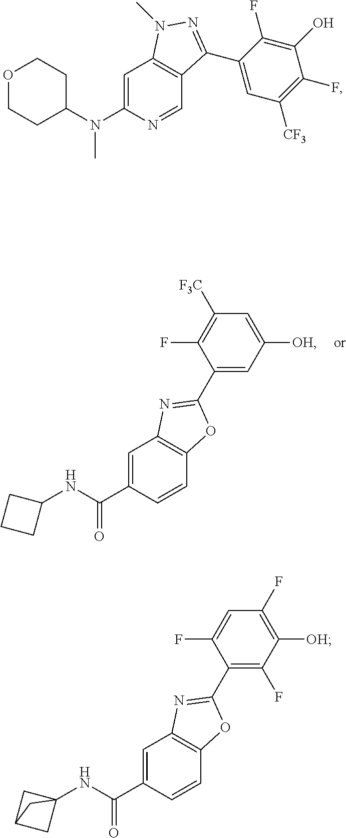 [SMILES: CN(c1cc2c(cn1)c(-c1cc(C(F)(F)F)c(F)c(O)c1F)nn2C)C1CCOCC1 ; O=C(NC1CCC1)c1ccc2oc(-c3cc(O)cc(C(F)(F)F)c3F)nc2c1 ; O=C(NC12CC(C1)C2)c1ccc2oc(-c3c(F)cc(F)c(O)c3F)nc2c1]
or a pharmaceutically acceptable salt or solvate thereof.

48. A pharmaceutical composition comprising a compound of any one of claims 1-47, or a pharmaceutically acceptable salt or solvate thereof, and at least one pharmaceutically acceptable excipient.

49. The pharmaceutical composition of claim 48, wherein the pharmaceutical composition is formulated for administration to a mammal by intravenous administration, subcutaneous administration, oral administration, inhalation, nasal administration, dermal administration, or ophthalmic administration.

50. The pharmaceutical composition of claim 48, wherein the pharmaceutical composition is in the form of a tablet, a pill, a capsule, a liquid, a suspension, a gel, a dispersion, a solution, an emulsion, an ointment, or a lotion.

51. A method of treating or preventing a liver disease or condition in a mammal, comprising administering to the mammal a compound of any one of claims 1-47, or a pharmaceutically acceptable salt or solvate thereof.

52. The method of claim 51, wherein the liver disease or condition is an alcoholic liver disease or condition.

53. The method of claim 51, wherein the liver disease or condition is a nonalcoholic liver disease or condition.

54. The method of claim 51, wherein the liver disease or condition is liver inflammation, fatty liver (steatosis), liver fibrosis, hepatitis, cirrhosis, hepatocellular carcinoma, or combinations thereof.

55. The method of claim 51, wherein the liver disease or condition is primary biliary cirrhosis, primary sclerosing cholangitis, cholestasis, nonalcoholic steatohepatitis (NASH), nonalcoholic fatty liver disease (NAFLD), or combinations thereof.

56. A method of treating or preventing a disease or condition in a mammal that would benefit from treatment with an HSD17B13 inhibitor, comprising administering to the mammal a compound of any one of claims 1-47, or a pharmaceutically acceptable salt or solvate thereof.

57. The method of claim 56, wherein the disease or condition in the mammal that would benefit from treatment with an HSD17B13 inhibitor mammal is a liver disease or condition as described in claim 54 or claim 55.

58. A method of modulating hydroxysteroid 17β-dehydrogenase 13 (HSD17B13) activity in a mammal comprising administering to the mammal a compound of any one of claims 1-47, or a pharmaceutically acceptable salt or solvate thereof.

59. The method of claim 58, wherein modulating comprises inhibiting HSD17B13 activity.

60. The method of claim 58 or claim 59, wherein the mammal has a liver disease or condition as described in claim 54 or claim 55.
